SEQUENCE LISTING (196 sequences)

SEQ ID NO: 1 - DNA, 1106 bp, Zea mays
ttggttttttgataatttgtttatctcacatgatggttctaacaatatgcgcagatcttatacatctctctcgtagtttttcataaactacgagaaagatataggttttatgaacaaatttacttttattttgtcatataatgaaatggtcaaaatataaattgtacatcatgatgagttatacaaatttgtagtccaaaatcttttcatttaaattaatttactgctttaaaatgtgatttttaattgtctttgcctagtgttgaaaaaaagcactcggcaaagaaactctttgccgagtgttaaaaaaacactcggcaaagaatctctttgccgagtgtcaaaaataaaatatttggcaaatagcttctttgctgagtgtttttttgcttagcacttggtaaagaacttctttgccgagtgtccgaaaaagcactcgataaaatatttggcactcagcaaagaaccgaattccagtagcgtgagtcagaagttggcatttgaaccctttgaaataatcaaacaaatcaacgcagttgcatatgaattggctttgccacatggtagtgcagtccatcgagtgttccatgtttcacagcttaaactaacccatggttttggtaacagaacacacaccagtgactccatcgttaccggattttgatactacactgaaggttccggaacaaatactggattctagattgattctagattgcttcgatcaaatggggatcatcaacctaacacagcttaaattatacatgcatgcgtacatttggtccttcaagaggaggcaggtgatgatcaaggtctctctagcggcctggtgtgcagccacgtacccatccacgcgtcgccgcgggcgacgacaggtgttggccaacctggacatcagacaagcacgacaatgacaggcgttcgccaagcgtacgtacaggagacgaacacgacaagtagcaccgtcctggccgagagcccgtcatgctcatgcatgcatgcatgctgatcatgcatactcgataaatatacagcagagagctgagctacgtacacaaacaagaagcttgcattgttcgttgctcgatcgttgcaggtaatggaga

SEQ ID NO: 2 - DNA, 922 bp, Zea mays
ttagtagagaataacacacatctccctaagtatcacaaaaaaattaattttaagaggcaattttagtttccaaagcgatttattaagatttaagggagggtatttattagaaggaagtacattgatcaaacagagaaaattccatcattatggtcccccatagaaatttgtcactcgattgtcgcaaaaaaagttaccagtaaatttgataaattttggataccggtctgttttaactcatcaagaatagcacgacgaaataatagattgcgtacgttttcattcataacatctctcactaaagcattttttacacacgctattaaaatggttcggaaactgatccttaagcggtctattcatatccatctatcgttctaagattcttcctgtacgtagattctttccgattaaattctcatgaaaaattaaaaacaaacaggtccttaaactagtcgccggaagttctcgcttttgctgtagttgtagtgtcgatgcatgggtggaaacgtggacagatagattcatgaaatgtggacaacccacctggtccagagcatcatgatacattgatacacgtggcacgctgcccactgatgccctcagctttgaagctaacgtgtcaagtgtggccggcccctgcattgcgtcgtgccaatcgtccaagtcccatggcaaaaacccagcgctttgtgccgccgccgtccgccggcccctctgcccttgtacgtgcacctagacacatcgtcatcgatcatcacacgcaatcgacacaagaagttaataaacagcccaaggacgcagagatcagctgatcgagaaggacttgtactactactcagtattgtcgtcacatgcacatatatgtacataaagagctagctacctgagctctacccaaggtcgcgttgatcgatcgatc

SEQ ID NO: 3 - DNA, 1945 bp, Zea mays
gattcagaacatctggtcagcttaaatgatgtggtaaagggagcattattcttttattttgttctttgttctgtgcaatggccatatattttcgtattgatatgctctcagtggaacagtctgaagctaatgtgaaacttcgatgttcaggtatgataatgtaccaacagagattaaccggctgagatgcagagttaactatcatgcattaaagttcctacctgatattgaggaaatggctgttaagcttgctgcaagaatgaggaacaaaactggcagcataaatccatacatgtacgttataattatccttttcgctgggtgatattgtgtacagtagagcttgtatctacccaagacactatccagtgcatagctgaagttgacgctctcaaagtttatttcacaatcaaatattgacacattttttttgttcttaaagggctcttcacctgagatttgaaaaagggatggtggggctttctttctgtgattttgccggcacacgggaggagaaggcaatgatggcggcttataggcagaaagaatggccaaggcgcttcaaggtgacgcggactggactagatacatctgttacattggactgagaaagaatccctaggagaaatgaagcaccgacactaactatacgcgctagttcgggaaccccgttttctcacgagacttttatttttccaagaaaaattagttcattttttttaggaaaatataaatctcttaagaaaatgtagttccgaaactagccctacaagtctaacgattttctcttttttgtatctgccacgcagaatggatcccatctatggccactggcgctgcagaagagaaaagaagggcggtgccctcttgagcccggtgagatcgctgtgatcctgcgggcactggggtacacgagcggcacacagatatacgtcgcgtccgggcaagtgtacggcggcaagaaccggatggctcccctcaggaacatgttccccaacctggtaggcaggctcaaatccacttcagattgtgtcaagctgaagccagtgataactgataagccactgccgtgcattgtgcatcgctgcaggtgacgaaggaggagctagcgagcgcggaggagctggcgccgttccggcggcacgtgacgagcctggcggcgctggacttcctggtgtgcctgcggtcggacgcgttcgtgatgacgcacggcggcaacttcgccaaactcatcatcggggcgcgccgctacgcggggcaccgcctcaagtcggtgaagcccgacaagggcctcatgtccaagtccctgggcgaccccgacatgggctgggcctccttcacggaggacgtcgtcgtcacgcaccgcacgaggacgggcctccccgagcccaccttccccggctacgatctctgggagaacccgctgacaccctgcatgtgcagggcatgagacgggccatccctcagatcagagtggccgtctggccgagtcctcgtcttcgctaggctacgactacgagtagtctcgtgcccacttccatcagcgcgcatttcagcgagcatatgcttctactagttaaacacctagatttgtatgtatttggctggcctaatctgtagtttcacatagatcacttcatgctgaaaatgtcttggacaaggcagtgaatgaagataacatcagtaacgacctgcttttccaaagctcctcttggatttaccaagctgagccaagaaatggctaacctgagttttgacttgcagccttgcaggcatgacgtgggcgacctgaactcgctagtcgtagagtttctttccacgcggcagctcggcagagctacgcagcctctctgcccgccacgttctcctgcaaggctgcaactcgaagcactgagaagctattaattaatggattctcgagcatgcggccttgttcgctaaacct

SEQ ID NO: 4 - DNA, 512 bp, Zea mays
gccagtgctaatgatatttacactagcgggctgctaaagaaaaccgccagtgctaaagatatttacactagcggttggtgaacaactgcctgtgaaaaaagccgatttctactagcccctagctagcactggcgacataaaaaacgtcagtgaaaatagctctaggatcgtcactatagagcttctatgtacttagtggttagaactgatattgtagtgcaccaagtgccgattttaattaaaccaatactaaatactagtaaataatactagtggtctgaattcgatttctatagtaatgtttgcttgcaagccgcaaatagagtaaacattcgtcgtcacagaaatccacattacatcaaggtccatggcggccggccacgtacccatcccacgcgtcgctgcggaggacacgtgttggctgaccggacagttggccgatcagacagtggacagaccggacaatagaagaagaagacgacgacggcggcggcaccgccgagtaggtgcat

SEQ ID NO: 5 - DNA, 698 bp, Zea mays
gtgattaagttgactggcaaattgcatagacgataatacactctatgcttttgagataaccactgtacaccaaccgacttgttgtttgaaggctgatctttaaccattactactgtaactaactgttgttgaataaactattcatacgacttggagactttgatgtatattatgttttgtacctatactattaactaccgcagatacgtatttacatctcggtaacatctgccagtcgcacaaatgcactaaggtgactgacagactgtagcgaccgatagagcgctgccttgttctagtcaaaattcaagcctggtggtcttctcgatccctctgcgcgcgccctgctcttttatcctgtgtgtctcctccacgagggaaggcgtcatgagaggacgacacgacgaccatgcagtgctgcgcaaccgcgcgcgtctgcctgttcactgagccccaggcggccggtccaagctattaccacgtcgcccccctccagggctgcgggcagcaacgtgtcgaccggtccgcctctgacgtgtccctcccgtcctctgcataaaagggtgcggctggcggggagcctcagcttcatatcgtcgcaagctctccgtgtttctctcgtttcttcactgctgccaactgtgctgtgcagtagttgcaagagctgattggtagctaggtacgctaggcaagtaggc

SEQ ID NO: 6 - DNA, 1355 bp, Zea mays
tataaattagaacggagggtatgtttttttacgtttccattatttgagtctgctcatcacgtttaatttgtttatacagttcaatgagttcagttacagcggatgataattcgacggcgtttatttgcatgcacttactttttacattacgcctagaatgtaatcggatggacgaatattacttgttttgtgtttattttcatatttttttctcttcggatacggatattatcgagttgtgccgaacaagattttgaatgtatatcgacatcttaaatatataactttgaatatacggatacgtatatagatcggatattgaatacccagctagactcagattaaattggatcttagttattttagggggtgtttgaatgcactaaaactaatagttagtggctaaaattagttgaaacatccaaacaccctatctaatagttcagctattagttatttttggaaaattagttaatagttaggtagttatctgttagctagctaattcaactaattataagttctagtgcatttaaataccccttagacggatcgaattagaacaccgatggataatatccataacattttacacctataaatatgtcctatatgtatatagatgtcaaaaacaggttgggcatgttggatgacccgtggcacgatacagttttaagcagtgccaaacatggcatggcggcgagctagacatgatacggtccggttttatttttgtatttttttttcgtaataatatctgtattatgcttgttcgaacctcacttgcttatgtggatgtgatagtatcaactcgctggcgttccgtgcatcgtaacatctatcacatcccacagatcatgtcgcttatcttttctgatactatagcagtagcacacagatgccggtcgaacccagaagcatcacgccatcaccccctccccttttcccaatcaaaaccacgtccgtcccaatggtaacccagcaccaatctcgtgttcgtgtgagacagagcatgcaaacaaacagcatgcatgcgcgaaggaaagcgtgcagcgcgcgggacgtgaaccgcgcgcgaaccctgccgcccgtccggtctcagctcacgcccagcgtccttcccaggccgcgatccggccatcctgacgcgcgtttcgtctgggccccaaaaccacgtccgccgcggcggcggcgcgcccacgggctcccgtgcccgccgatagtttgccggaccctgccgcctcctataaatgcagagccctccgccagcgtctcccgcatctcatcttcttcctccatccgcactactgagagcgacaaagctagctctctccagggatcggattcggatcacc

SEQ ID NO: 7 - DNA, 1941 bp, Zea mays
aagggactcgtggcctacacacgcgcacaataatcagttaccatgaattattttatatgcgaatatcgacattacacatgaataatgtgaatttgaatttccgcagcatgcaaaattcccgtgagtttgaagggcgataaaaacgggtggccaaaattggggcggcaatgcaagaagataagcctatccatcccttgcattggtattgtcatctggggcagggcaggggagggcaggagaagctatctattggccatctatccagcagccgctctccccagccccactctctccatctagagctccctaactgcaccggccgccacaccaccgtaccccgacccctcgacaacaatggccaccgtcctaggcagcccccgcgcgccggccttcttcttctcgccgtcctccctccgtgccgcgccggcgcccaccgccgtggcgctgcctgcggccaaggtgggcatcatgggccgtagcgccagcagcaggggcaggctgcgcgcgcaggccacctacaacgtgaagctgatcacgccggagggggaggtggagctgcaggtgcccgacgacgtgtacatcctggaccaggccgaggaggacggcatcgacctgccctactcctgccgcgcggggtcctgctcctcctgcgccggcaaggtcgtctccggctccgtggaccagtccgaccagagctacctcgacgacggccagatcgccgccggctgggtgctcacctgccacgcctaccccacctctgacgtcgtcatcgagacgcacaaggaggaggagctcaccggcgcataatcattcatgcccatccatctctatctctgtgggtcgtcgttgtgtgtaatttgagtacgcgcgtacacgtactgcttttgtttcatttgagcactgttcgtgtaagcgtcgagttgccctgccatcgtctctctctctctcgatctctactatttctgtgtgtgcgcgcttctatttttccaatgttgagttacgcatatttgtccagtaaccttgttttgcttaatcacacacagctcccaaattccagtcgttctgtggtgacggttggttgtggggtgtggcggcgacgtcagtcatgggagaagagatccgaatgcatgggggtgttggcgctttctctgtccctctccctctccctgtcttctcgcgcgctcgccgtcgtcatggctcgacgaacagacattcactgccttccccgcgcgcttcagacagaccacgcttgtacgtagcgccgcttctctagtcgccggagcaggcacgagtgggtggcgaagaggccacggcccgacctgctagcgccagcgaccgcatctcgccaccagccgctgcccgctggccgctgccaccgtatgttgggttgggctgaagcctgaagcccgtgacctttttctttttctttattctttccttttcttttgccggacaagacaagtgatgttgcaaaatccatataaaatcctagagaaactataaagtttttttttgttgaactccttataacaacgtctaccctttaatatatggtatataatttcacatgttttagtataaattgtttactgtatttctatgtctatatgctcttggtaagattgtttataaattttaaatttaaaatcttaaaagtcataaacttatagaaaaaaattaggatcctaaacaattttaaacaaaaaaaaaaacttttcaattacaaaatcgtatgtgttctttatgcatgcatgcaactgagaagctgcccaacggccagcgcagcagaagggcagagcggtgggcggccacgtcgccgccgcaaaggaactgacgtgtcccggcccgacgacgacgacgcgaaccttcaccgcgccgcgctccacctctcttaagctaaatacgctagtgcaggcgcaggcatccagctgcgacaacgta

SEQ ID NO: 8 - DNA, 1188 bp, Zea mays
gagcgacctcggactcagcggctccgcctgtcggcgagcgcacgccgcgtgcctgcccgcggtctctcgtcgtctccagaagatgcgccccgatactcgtctttccacccttcttctacctccttccacccccttctccccacgccccatcggatcgcacgcgcaccgcaattgttgttcatcacctctccgaagaagcgtcacagagcaccgcccggaggttccccttcccgtcgccgtcgcttggccaggtaagtaatgactaatcgcctgagtctcctgcgcctcattacttcgaactgtggtttcgttagtcggacgtcggagcaatgctatgaaccttgccattccatgttacttggtgctgcgtgtccacattcgacgccacatttttttcttgctagccaaccattgcgcgtctgtgttacagttccaccggcgatatacattatgcgatcatttgatgtggatattggtgtttttttggttagcggatcaccttagttttgcactgaacatggaacaaatgtggatgttggtgtttttttgttagtggatcaccttagttttgcactgaagatggaacgaggaaatggggaaggggaactgaacgaagcacatagtttcagagactatgtgcactgcagagactatgcgaatggtcaagggaatttctcgccttggtttcctaatcctcgcttccttcttgtttcgaattcggtgtatatatgtggtttattcggcacgtttaggaatttagttggggaatggggcgcagtatgtcagggttgtgtcatctgaatatataatgcaagatctattactagtttaaattgacttattatgcaagtcgattaacctggctgggctcagtggtgtaccatggaagctttgtgattgtagccgggcagtagaacgctaaacctgaggtttggtactgtagcactcggctagtcagctgtatgccaagatgttacttactcgatactttctgccatgtctcatctgtgttaaccagggtgtgtgccacatagggcttctttccatgctgtttgttaacctgttcacacttcttccatatatatattcataatagctcgatctacaattttcctgtcttgagcttctttgttcctaaaaacgaattgtataccagcaatgtatatctgatgcaatattgtttgtagg

SEQ ID NO: 9 - DNA, 1507 bp, Zea mays
ctacatttatgttatagaggcgcaagtagaagaatgtgttataagttgtacatcggaaaaatagcatgtaaatctatagaatcaattttcatctttcaccccatgaatttgaggtatgcttatatgataactttagaaagtggtggaatgtcatattctaaaaaaaatagcctatttcattagtaagattccaattcctcgaaatgaaagaaaacaaacggggccttattagaatggaattcaattccaatgatccaaatggggcgtaagggatttccactttcctaagaaaaattagctcattttcccttggtgtttaataattatgttttgttaaaaggtcagcttcccgagaatgcgttgtgtaaaagcccagtataagggtctgtttggttgggctgtggctgtgaaaaaagttgctgtgggctgtgagctgtggaaaaagctgctgtagactgtaagctgttaaaaagctaaaaaccgtttggtggaaaccactaaaagtcgttaaaaaatcttcgatatatgttttcacagttccatccgaaaagccactaaaagcaggtccagaggtgctttcagatttgcactacgagaaagtcggtttttagaaaaagctgcttcctggatccagccctttggktggcttttggctttwaggggcacaaaagccaaagccaaaagtcaaaccaaacacaccctaagtcgcgtgagtgggccatttatctattccctatgcaaatctcctgaaaaattacaacattagctcgttaaaaagtaaaataattttaaaaaattagagactaattatttgataactttatagttcatcacattttcattgaaaatgttttattttttttgccgaactgatctacacaatatttgaatctacacttttggcttgggctcggagaaaaatagtgcgtgcccagccctttgctctttccccgataacaacgcggtatgtggcctaacgcccaaatccggcccatttattccggtgcgtagatgcagacgcgggagcaggtgccaagtcactggctcactgctgcaggtggagtggtggacactagtgccgcggttcatctgacacgtgtcgccacgtgccgccatggcagcacctcagcccggccggcgggccgactgacgtcttgggcaaagcggcgagcgacgcaggcggcgaaagccatccgatttgacccctcgctagacctttcaagaacgaacgctgtgctgctcagatcagaccgtgtctgcctcaaagcgatgccaggacgccacgtccaagcaaagcacccgatgccattgccacctcccagcactcacgcgtgagcgtgactataaaaaacgcaccctctgcatccgcccccgtctgcctgccctaccgaatctttcgccgtcccatcagcccagcaattcttcgctgttcgaggacccctcggtttcgaccgaagcccagcaagccgaccacacaccgctgccgttggttccgtcccaagagatg

SEQ ID NO: 10 - DNA, 910 bp, Zea mays
catacgatttcctaagcggaatctcgagacgaaccacatttttcatccggtctatgagcgcgctcaatcgtcagtgacagagaacactgtaggtccttaactggaactaacaattcagaaacaatcctaaatgtttattacactggatggattttatttggatagatgatactaccgttcgtgggctaaaaagcaaggtaaaaactggcctaacgcacgtgatcctcccatggtctcagaagaaaaccactcgcaaaaaaaaaaacgcagcaatgaggctgctttcagttgacggtgtgcacgactcgctgaagccagcgtgattttgtcccggttcttggagcccagcaaaaagggcgcctgaaattccacaggttgaaggggaaaacggcaacaaaaggggctccgacatgcggtgcagaaagtgaaaacgcgagaaaagcgcccgtcctttctcgttcccttccgtgcagttgcaagtgttgccacgccgcgccgcgacacaccacgcaggaggcagcgagcgcaataatgcgcacgcagaggagcacagcggcgtcactgctgcggcagtaatggccgcctggttccacttccaccacggtgtgcactagcgctacaccgaggccaccttttcttggttccttcaacacgcctactccggcctccggatcgggcaccacgtctgccccctgctcccggaatctttgccaagcggcgccgctactgtccggcaaggcgaccccgtctctcggccggcgttgatctggtgatgcgcttttcacggtgtttttttttttttttgtttcactcccccctccgtcctgtgtgattgtgctgcagttttgtgccggtgctaacggaaaccggaaagtttgcgtctttatgttactgtggttgaggcgggcgg

SEQ ID NO: 11 - DNA, 1131 bp, Zea mays
ccagccatcgtgcttgagtgaatgtttttttgcgtgtgtgtaggtgtgcgtgtgtgtttaattgatataaaataacccaaccaaacatctcctaagatttccataagacagagaggtgaggaatagtattagttctcaaatgaattcaatcaactaaatatcaccatcatatcaacattaataatagaacaataaaattttcatgatagagaggctggtaaactttattttttcatttgattccagtgtggaaaatagtaatataagcagtaaattttttcttaaaaaagagtggaatattcacctttttatataaattagttattcttcgaagcgtcacacacggatgtcctaaagatattcttcaactgcaacacaagcattctgaaaaggttagtaacaatcagattaatgttgtaaaaatatgtgacacgaaaatttaaagaacgcgaagtacaatacattggtaagaaatcaaagacacatattaaccaactattagagaacatatacatgtttcttctaatcctggttaggacataggacgagggtttcacttctatcacaatgtgctgcaactttcagagttaatcaaatctgttattcagtgttatgcagcgacaaattccaaatgtataatcccacgatgattcatgtgactccatttcgtctgtaattgatgcgaaactgccgaattgttactgctcccataagttaattgtaccacgcttgatgaagacgttcacgtatcttgtgttcgatttatgtagccgttattcagaccattgcagcgctgagcttcaaggcaattagttttgccccgcgatgtgaggcaggtgatgacacgcgcccttcgggccgatgccgacgcgccaccgtgcagactctaccgaggatgagagcagcgatcgagagacgtggagcgaggaggagactttaggagaggagagaccagagccttaagcggtgacacacccaatgatcaggaccacgcttgcacgtgtcaggtgcctctcctccacgcgtcgcgcgcatcacactgatactgtcatccagctcgacgctatataaagtggtaagcatcttcgtcctcggggccgccccaacggcccttgcgatcgccaccacgtc

SEQ ID NO: 12 - DNA, 563 bp, Zea mays
cattgttatacatcggtgatgcagatcttcgcttcgcatgggtggcaatttagtaacagaattgctctatgagttattgcttttagtttctgtctttccccaactgcgtttgagtttgcattgtcacatgtggccttttttggttgtaagacttgtttgatcggttgtggtgggctgtcatcctttggatgtaggccggaacctcttttcccattatctaaaaaaatacatcgtcatttccagtgagacccagcggtcgtgtcatcagaaaagcggacacgcgtcgcgtcgcgaggccaggtgggttatgcacggacctgctatgctagccatgctccaggctccagctccacccgtccaccgacttttcttctcaaccaggctgcctcctcaccatgcacgacgacacgtgcgatgccatcacgcatccacactcacctgttgcctctatatccaccgccccaccgccgtagcaccagaaagaaatgtacagtctacacagctgaccagagagctagtgcaagcgatctagcaagttttaatcttggaagaagccagctagg

SEQ ID NO: 13 - DNA, 991 bp, Zea mays
atcatcaccctaccccgagctgactcgggccgcagggaaccagaccggtgtcccatctggctagctccgccagataggcaatgaaggcgccccgcatgctctgtgacgacggcggctctcagcccccttacggaagcaagaggacgtcagcaaggacccaaccgctccgacagctgtccctccgccaggctccatcgctcctccgacggccacgacatcgcaccagctgggtgccaaaatctctccggctgccacgacggcatgtacttagggcgctagctctcccccgctagacacgtagcactctgctacaccccccattgtacacctggatcctctccttacacctataaaaggaaggaccagggccctctcagagaaggttggccgcgcggggacgaggacgagacaggcgctcgcttggggccgctcgctccctctcccacgtggacgcttgtaaccccctactgcaagcgcacccgacctgggcgcgggacgaacacgaaggccgcgggattcccacctctctcacgccggtctccggccgcctcgctctccccccttcgcgctcgccctcgcgctcgatccatctgggctggggcacgcggcgacactcactcgtcggcccaagggacccccggtctcgaaacgccgacaatatgatatatagaaactataatgatacacatgcaattaactagtgctgataatatatttacactgtagatttttccgataagtagaaaccaaatatttcaccgccacaaaaggcttcatcccatcagttacccatgtagcaaaaccaaaatacttgtgtgcgagaggccaagagcccaaaccaccgccaaggaaacccccagcccaccaggcgccttcctctataaataccctaactccggtggccgaagttcctcacccattcactcactcatctcaaggcctaggtagcaccgtagcagctactagaagcagctagccagaacaactcgtc

SEQ ID NO: 14 - DNA, 2519 bp, Zea mays
cttccgataaaaatatttggaaccgcatcctgcatatttatttagaaacattgcatcgataaccgttgcagagcaatatacagggtactcaccaactaaatccataccatacagtgacaggtgaaattggaatacttgatcagcttgaataacagagagcaaagataaatctagccctggtaataaccatggctcacatcttatgtacaagagccaattctaaagacacagctgcgggatgtagcagttaggcaaggaaagaaacaaaattaccagctaactgctaagtgtcaaaacattgctttacactaaagggttaaatatttgccaagaaggtagtgggcacctctcctctgcaagcagcagcataatctagggcgaggcttggtgcagcttccttgtaggggcatatgaacttatccatgaaggcactctcactcatctggacagcagtatagtagctccggtcctgctccaacaatccattttccttcacgcctagtttggatcactgtaattgaattccattctaataatagtaatttagacatatatcaattaagataattcacttttgtacaaaatatatttgtatactattattagcaatatatcctaaatatttatgtgctacatttttactatagaggagtagaacgaagagtgtcatataagttatagattagaaacaaattctaatcatgcataaaatcatttcccatcctccaccctatgaatttgagataggcttatatctgaactttagaaagtggtggaatgtcaaattccaaactaaataagttactttattgagtgaattctaattcttttgatttgaaggaatccaaacgccccgtcaggtaattcacaacatagtgtcggggcatgaggcggcgttccaagctatatgtaagcagagcaggcctgtgagcaatgtactctggctccaacccaaccttggtgatcaggaacgcggatccgggcgtcgccgggggcgcgaccctgtgcttgccaaattgaaaatttagcaaagtattttttttaccttgttgattatgttggataaccgcatgtccctaatttgaatgatattgctccttatctctaaacgaccaactgtagcttgttctattgaaaactcatcagaataatccatagagataacatcaccaacaaggactgaatcatccagagatgcatcaatggccattggagctagggaagtcggcctaactaccttcaacccgacttggccaacttgtacagtagaagacaccatggaaatcgatttaacggactctaagggagtgtttgaatacactaaaactaatagttttagtggctaaaattagttggagacatccaaacaccctagctaatattttagctattaactatttttagtaaattagttacaagttagctagctatttattagctagctaattctactaacaaatttttagctaactaacgcattcaaacactgtctaaacaggtaatctaggcacgcgaagtctgactggccagcccaaactcagcctttgtagtagagagagccgaccttggcggcctgcacgacatttttgaactttttttatcggtttggtcttcattactttatattctttgtcaaccaaatactcccttcctcttaaattataattcgtttgattttttttatgtaaagttttatgtatagtccacctagttaactttaaaagttggttagtctagattttatggaggccgactacaatagcaaatctaacaataaaaaagttagctagatcagatttcaagaacagtctgaccgattttgaagatttgattcaggttaattctacgattttgaagatccgacctaacctaattttacgttcagactaaagacatgtttgtttaagattatgatatgtctagttatataatttaacttattttaaactaacacttaatttaaaataagttagattatatgatcagaatattatatttatataattccaaacaaataactctaatatcgattctggtttatattgtataatttttatgttgaatattacggcgcgccaacaaattgcgatcgttatgtaatcatgtatacgaccgactgtactattcataaaatataattcaaaataggagatgctcgagatagcctggcaagggaggaggtatcggaggcggcgtcgcctgccgcgagcgcaccatgcgtcaacgctcaggacctctcgcaggcgcgctgttggatcgtgagcaagcacgcgtggcaaatggcatgcattcctctcgtgcaagtgccgagctgagggtcccgacgcggggggcaggacgtggcagagcgccgcccaggcggacgccaagtgccgagctaccagggcaccttctttattccgtccttgctcagtcacacctcgctctcgctcactctcgccgtccgcacagccgctcatcgtctcccactgcctgccctctccctgcgccggccggtgccccgacgcgccatgtcgtac

SEQ ID NO: 15 - DNA, 1264 bp, Zea mays
tcatcggtactcgcgatgtcgtgcgcgtcgacgactactatatcaaggactatgacgtcatcttgacaccagaggtgacacagaggaggaccaacaacaataaccactcgccaccgtcggtcggtctgcctttaattctcttcgcaaacacatacacttgcttttttgtttaagcaagcgtgtatgatggtgcgtacctgatgactgacaatgtacgagggaacttctaccggcaggtgtaacacgtgctcttcaatgataagatcatgcaaatcgtgtcatatatcgaagcctgcatgatactgatggttgaaatatagtagtgaaacagctaaattaaaataacaaaatttatgtatggctaggattacaaataaattataaaatatttttttataacaatataagacacattttgtatataagttattatattattatatgtttctgttgcaacgcacgagtactcacctagtatatccataggcttttgatcctctacgtcgagtttttgcttgcatgagagtcgacctcttctcttttatcccttcttctctatcttagcatttgctcgatcaactgtaaactaattattatacgtacttcaagtcatattgaaaccaatggcatttaaaaaagtaaccatctgaatggaaatatcaagctaaataaagaagttacgggagtatgtgctgagaaaacctagacacattcagagaagttaccaagcaagccttaaataacaatttttggtgctttattgcagatagatactcctataggttggagagaagggcggcgatgctaatgcaccatgcatgtgtgtctgcccatatactctatcttatctgaatcctaccaaactctttaatttgactctttatgaatggaattcccatatgaattctataacacaacagtacgacgtctactggagcatactgccaagcacgctagccgacggctcggcgaccgtctgaggctgtccatgcatgcaaatgtgcacacatgtacgccccttttagcgctggcatgcacccacgaccacgacacggctcccgtggccgtgaatctgacatcccgggactcccgcctccggccgcgcgtgccgtgtcagcctccatcgagcggtgtactccatgcaagcctcgacccgggccacgtacccccctcccctccgctacgtgtcaccgctctcgtcacctatatatggcggtcttgtccagtctttccacttaactactccgtcactttgtttgcaaag

SEQ ID NO: 16 - DNA, 623 bp, Zea mays
ctcacacaaatctaaatagtaaagtagatctggttacagagcagaaattttgaagtgtcatagataggctgagaatatgtagtcacgagcaacacattactacttgaggccatcaatactgttttttcttctccaacctatgtaactatgttattcttttcctccttaaatgtaaggcaaacctcctatcctttttctttcaaattcattgcattaactgagtggttcttacatgtagaaatgaagttggagcaaagaggcttggtgcggaaccgaaaatttgttagtctataaatcattagagggcgcctgaagttcgccaaatgttctagtagattttagtaaccacccacgctagctgactcgaccaccgtctgctggctggcgctgtccatgcacatgtacagcccctggcgctggcacgcacccacggcatacctcccatgactctgacagcccgggactccctccgctcgtgccgtgtcagcctcactcgagcggtgtactccatgcaagcctccacctcggccacgtaccccctccccttccttacgtgtcaccgctctcgctccctatatacggctgcctggccaggcctttccacttcactactctctctctct

SEQ ID NO: 17 - DNA, 1950 bp, Zea mays
ggttcaagatatgtatgtgatgaacataattttactcaaaagtgatggtttcattgtactaactagcatcaatacttatgccagagtggagcagtgtgtctatgcactactccgtacaagagacatgtccctcacgctacaaggagtatgggatcccagttgactggttgtctgattccggagtagttggcaaggtgtgcatctctagcatacactccaatttcgccaaacatgtgttaaaaacatgcatttttaattccttatgaaggtgggtatgattttactcagagtatgcacctaaatgaactcaataatttatttgcatgttggatatatcaaattggcatctgttcagcttgcgaacaagtatatgaagagggttgcatcagaacttgatgcgttggaaggcactgagaaagagcccaatagagagtttttgcttctccagggtggcagggtgtgaggttctctttccgtgttcatcaggtaaaatcctcacgccaatattttcagaacccaggtacttcacaggtgatatgaatatttcgaatcagtaacttattcatctaccttctgcagtttgctgggggatttgatgcagaaagcatgaaagcttttgaagagctaagaagcaagatgagtacacataaatctgctccacagatatctgaaggctgaagcatgagttgaacacaggattcaacacacatctggtcttacttgctggatactgatttttagttgtgtacaaaagagaagtttaaattccagtgtggtgtcaagcaagttgtatatgctaagagaatacaagtcatcaggttatgttcatttattttttatgaaagtaagggcaggacaggttatgttcatgcatataacagttttagctcaagtgtataccttttcattttaaaggctgtaactgtaaacaggcagctccagaaaatgaatcacttgggcatgtttttgttgctacgcatatactactaataaaatgtcaaaagcgtccattcggttagtccggtacccatggagataattcgtctgaatgaaccttgccgtctttgttaaaagaaaaaaaaattactttgtgcctcaccgcattcgatactttgaagaaattgacccgttaccagttaccacccacgcacatacagtgctagaacagcagatctgacttgaagaagcatatatcaagacaaaagtccacctgaacggagacagtcgaggttgtggcggagagctccacctgaacgccgaagtggaaccgaacagttccagtggttggccgtggcgagctggccacctactagtctccggggagggggctggctccagcgcctccgagcgcggtgccagtgtcgccgtcgcgtgggttccggttccggccgccgccgcttgccggggaatccaaacggcagacggggtcgccgtgaccgtgaatcgtgtttacttgggccttatctaggccacaacgtttgtgactctgtgagcttattgtttgggcccatctctttggcgtgtctgagaacgaattttatcatcaagcccacgaagccaaaccaggatcctacccacctagcacatactagtgaagaaggtaaacaaagtgtattttcatgttaattcacatgcgcgtggtcgacaccacaccacacgacttaaaactcggtgttatgttctacgaccacaagagccatgttaattttaagccgtgtctgcgccatgatccgaagagcgcgtacagtgagagaagaaacgcacggccgccagcagctgccgcgggagcgaggcagcacgtccgttggtcggcccgggcgggagcgaggcacacgtcgcgcatgcacgcgcggcgcccggacacgtgccggcgcgggcacgtccctctccgcgccttatactcctccccgccatttcccagcagccacaacaagacagagagatacccga

SEQ ID NO: 18 - DNA, 2112 bp, Zea mays
ctaggttggtgagatccttaggttttggaaggctttatgtggcagctctgaagactccaacatctatggcctctagctatgcctccatatgtattttgtaataagaaaaatcatctcccttaaacatgggagagggttcatctccatcgaacatctttctttaggtggtgaagcctaaaataatgagcacgacactttgatactaattgaaaggattactatgcccaagaggtagggtggattagaccaatctaaaatttcttgtagcaataaaatcttatcgaatatcccacttcaccacttgtgcttaagagtgatatatattctatcacaaaaggttttacaccctagtttgaaccctattctagtatgttagttctagtaatgtaaagacaagaattaaattgctaaaatgcaaatgcttaagataaagaagggttagaaacatgacagcatttttccaaagtattaaagagccgggactctccaatagtcctcgttggagcacccacacaaggatattgctccccattgaacttcacaaggatcaagggctctctatgggtttgttcctcgccactctagcatggtgagtcacccacagttgtttacaccttgagtggtgatatacactagatttcttaagtgtgtatcatcagacttatttaggtcaaactatcattcatggcccctttatagtatagtcaaaggaaaaaacaaaggtgctatcactagtatatatgtctatcaactctattaactattagaactagccagtccttaacctttttgctcatccttctaaaatcaataattgaataccatcaataggggcacaaaaactatatagccaattattattactcatagccaataatttagtgttgtcattagtcactaaaactagatgctttcatatctatcatgctcgtgtttgcctgatccaattcctaggtcatgtttgggctccacttagagcttgtaatggcatcacaacatgatctgattaagaaaatgttgactaaagaccttctcacccacatataaaatgccatgacttcgctcaaacttcatcccctagtctcctactacacccatgtctatgcttcgcaacttcacaagtctaccacatgtacttgcaaagggctgattctcctagctcccatgtcgtcgccatctttcccctttaccaccacaaaatcacaaatgtgctcacttattatccccgcgtcttcccttccctaccctaaacactatgggcaacaacgtcgcatcacttggtggtccttgtagatctagacgaggtctcctccccgttttgtgcttgttgatatagtgatcttgtccttgtcttgtcgctgtcctttggagtcgtcgcagagaggcggtgaaaattctagccgcttctagaaggtgcggtcgccggtcggatgagagtcgagtgagtgcaggcctataaaaagtagcaattggataacattttgaaaaaaatgaaagaaataaaaaaactagggttaaataagattctaaattaaatctaagaaatctatagacattcggaaatctatttatcttttgtactcctttgattggatttctagattttgacgtttgtgtaattgggcttgacagccccatcaatacaggcaggccagctgatttcgggtgccgctgatttggcccatggaggatagagcaggcctttcaacaggcaggcagtccatgacgtagtgcctcggcctcaaagcccatgcgattaatatccactggtacgtacgcgacagccgagggagtcactgagtcagtgccgaacggcctggaaaccccccgccgtgtgccggtgtccacgcgcgccccggtgcggccgccacgaccgcggacacgtgccgggcctcgcacacgcgtcccccacggccccctcgcatcattaaaaccgcggtgcccgcgccacggatccccggcacttcattccggtttccggatagcttacctcccatcgacggtcggtgagccgttggtgagtggcgagacagcgggtgcgctgggcaagaacaggcgacgaagaagatgagcacgccggacacgcggacgctcaaggacgagctgacgagc

SEQ ID NO: 19 - DNA, 456 bp, Zea mays
atggtgactgtggagggaagaagaggctctagtgatcagacaggcgacatgaggtacgtgagtgacttccccgtctacgatggccgtggctccgacgccgtcctggtggctcgcgtgcagggcgtcacaaccacgttcgggaactccaaccagttcttcaccgtcgccttcgaggccggcagcaggctcaagggctccacgctccttaccgaaggagtggtgacggatggatcagatgaatgggcaatctacggcgggaccggagagtttgctatggcgagaggtgtcgtcaaaagaaggtaccttgccgatagggatggcgccgggaacactgacgagctcagcatgcaggttttctgcccggtgtttggctcatcacagcaaccaaacaagcaggactccagcatctctgtcaccaagattgggttatggggcgccgactttgatttgatctga

SEQ ID NO: 20 - DNA, 492 bp, Zea mays
atggcgcggttcgtggacccgctggtggtggggcgggtgatcggcgaggtggtggacctgttcgtgcccggctccaaggacatcagcaacggctgcctcctcaagccgtccgccaccgccgcgccgccgctcgtccgcatctccggccgccgcaacgacctctacacgctgatcatgacggaccccgatgcgcctagccccagcaacccgaccatgagagagtacctccactggatagtgattaacataccaggaggaacagatgctactaaaggtgaggaggtggtggagtacatgggcccgcggccgccggtgggcatccaccgctacgtgctggtgctgttcgagcagaagacgcgcgtgcacgcggaggcccccggcgaccgcgccaacttcaagacgcgcgcgttcgcggcggcgcacgagctcggcctccccactgccgtcgtctacttcaacgcgcagaaggagcccgccagccgccgccgctag

SEQ ID NO: 21 - DNA, 1278 bp, Zea mays
atggcgcagcttagagcagccgccgacgagcagcaggaggcggcgatgcacggcgtcgcctccggcgacgggaacgagagcaacaagaaggcgcgcgcggggctctgtggcctgctccgcgagcgcaaggtggtggacctggcgcgggccaagcggcggctggtggaggttccctacaccgcgacgctggcgcacacggccaacgcgctcctggcagcgcgcgtctctgccgtggccgtggctgcgccaccgggccactggatcggcgccggcggctccatgatcctcgagtccgacccggccaccggcgccgtccgcaagcactacatcggcatggtgaacatgctcgacatccttgcccacatcgccgaggccagcgacgacgacgaggcggccgacctcgaccgccggatggccgtgccggtgtcctccgtcatcggacactccctcgagggcctcactctgtggacgctccacccgagcaccagcgtggtggactgcatggagacgttcagcaagggtgtgcaccgcgcgctggtgcccctggagagctcggcggacaacgtggtggcgctggagctggtggagtcggcgccggggtaccggatgctcacccagatggacgtggtgaggttcctcagggcgcacggcgcggagctcaggggcgtgctgttgcgcaccgtgcgcgagctcggcgccgtgaacgacaccgtgttcgccgtctctggcgttaccaaggtgatcgacgtcatcaaggcgatgcgggcggcgtcgctgaccgccgtgcccgtcgtggacgccgtcggtagcggtacagagacccttcaatacgggagcgggcaaagggcggtcgagacgttctcggcgacggacctgcgggactgcccggtggcgcggctgcaggcttggctggggatcagcgtgatggagttcaagaggaaggtggctgagtaccgcgccagcaccaggcttgtggtccccggcgccgacgccacggacactggcacccctgtcgccgggtacgccgacacccccgcggctgccgccgccactgacgaagagcagagcgagcagcaggagtcggcgctggtgacgtgctcccaggagagcaccctcggcgaggcgatcgaggcggcgacatcgaggcacgtgcaccggctgtgggtggtcgacgaggaagggctcttgcgtggggtcgtgtcgctcaccgacatcctccgggcagtgcgggaggccgcgctcggcgaggacctggagctgcacagcatcgtgccgtga

SEQ ID NO: 22 - DNA, 975 bp, Zea mays
atggcggagaaccctgctccgagttccccgctagtgacttctcccgacgccgctgcgccgcctctgccagcggagttcaccaagctcaccttccgtagcctgtacatccgtcggacaggtccaggcagccgggagatgactgtggacgggaggccgtctgaccagctgggacgacggttcgtgagcgactttcccatatacgatggccgtggctccggcgcccgcctcgtcgctcgcttgcagggagtcactgtccaaatcggcagctcgcaccagctagtcagcatcgtcttcgaggccgagaggcttaagggctccacgctgctcaccaacggagtgataacggatgggtcagacgagtgggccatctacggtggcaccggggtgttcgccatggccaccggtgtcatacgaagaaggtttctcgccggtagcaacgacggcaactctgatgagcttaccagcatcgaagttttctgcccggcgtttggctcggcacaacaacagccaaacaagcaggactccgtctctgtcaccaagattggggtatggggtggaggaggagggtcggcgcaggacatcacgacgacggagccaccacagcgtctgcacagcatcaccgttcgaactggttttgccgtcgactccatcgagttcacgtatactgacagaggtggccagaggcgcactgctgggcgatggggtggacttggcggaaaccttcgaacgatcgatcttggcgacgccgaggttgtcagggaggtctcaggaacatacggcacgtttgaaggcgccaccacgctgacctcgatcagaattctcaccagcagcagaacctgggggccatgggggatcgaggacgggacacgtttctgcatcaccgcgccgatcggcagcagcatcgtggggttctatggacgctcgaccagcaggctcgtcgctgcgatcggtgtttacctgcgccaacaactctga

SEQ ID NO: 23 - DNA, 573 bp, Zea mays
atggctcccaagcggcgcggaaacaaggtggtgggctccgtggtgaagaccaaactagtgcaggagaccgtggaggtcatcgtcgccgacgacgatgggttgcacgcggagaagcagcaggtcccggaggctctggccctggcgcaccccaccgtggacgtctctggctccacggtggtgcatgtcgtcgaggtcactgccaaaagaggccgtggtggtggtggtggggaagaaagcagcaacaccaacaaagaagacgaagacgcagcagccgcggcggcggcggcagcggctggggcaggccagctccggaggcgacgccgggatgggcggcgtgggagggtacaggcggtacgtgtggcgcgtgctgaagcaggtgcacccggacctgggcgtgtcggggcacgccatgcaggtgctggacatgatgatggccgacatgttcgagcgcctcgcggaggaggcggcgcgcctgtccaaggccacgggcagggcgacgctcacctcccgcgaggtgcagagcgccgtcaggctcgtgctccccggggagctgggcaggcacgccatctctga

SEQ ID NO: 24 - DNA, 351 bp, Zea mays
atgccgtccaccgtgaactccgcgtccctcgaccagaaccttgagctcgccaagcaatgctcgcgagaggccgccctcgcagggttcaaggcagcagctgtcgcgaccgtcgcctctgcagttcccaccctggttagcgtcaggatgctgccatgggccaaggccaacatcaaccccgctggccaggccctcatcgtctccacagttgctggcatggcgtacttcatcgcggcggacaagaagatcctctcgctggcgaggcagcactcgtacgagaacgcgccggagcacctcaaggacacctccttccagggcaccggccgtccgcacccagggttcttcaggccttga

SEQ ID NO: 25 - DNA, 390 bp, Zea mays
atggcgcatagcaagcgcgaaatcaagcccgggagggcgcataccaaggtggccggcgacgacgagatgctgcggaccgggttcttcgacggcacgccgctggagggcggcaagatcgccgactcccagcccgtcgacctcttcgccgctgcccgccgcgtcgccgacgccccgtactcatcgcagcaggaaggaccacacgaggaggggaccaacaagaacgccgtcatcgcggagtcggagcccgtcgacctgcccgcgagcgcgcggggcgtggcggaggtgaaccgtgctgggccggaggagcagacaggccgcggtcggcagggcatgaccgagcccaccgccggtggacgtcgcttgggattaggtcgcccggccccggcttaa

SEQ ID NO: 26 - DNA, 1737 bp, Zea mays
atggctgaacaattctataccgtggcatcggatagtgaaaccactggtgaagacaaatcacagccttcattccctgatgttgctgttggcattgacattgggacttccaaatgcagtgttgctatttggaatggccaccaagttgagctcttaaagaatacccgtaatcagaaagggatgaggtcatatgtcatgttcaaagatgatactctttcagcaggagttactggaggtgcagccaaggaaaatgcacacgaggaaagagacatcttgtcaggaagtgccatattcaacatgaaacgcctgataggaagaatggacactgatgaagtggttcaagctagtaagaccctcccatttcttgtgcagacattgggtatcggtgtgagactattcattgcggccctggtgaacaacatgtggcgatctacaactccagaggaggtccttgccatctttctacttgaattgaaggccttggtggagatgcaccttaagcatcccgttaggaatgctgtgcttacaataccagttgcgttcagtcggtttcaacagaccaggattgagagagcctgtgcaatggctgggctgcatgtgttgaggttgatgccggaaccaactgctgtggcgcttctgtacgctcagcagcagcagcagcttgtgcatgacaatatgggaagcggcatcgagaaaattgctctgatatttaacatgggtgctgggtactgtgatgtggccgtggctgccacagctggaggtgtttctcagataagagcattatcaggatgcacagctggtggggaggatatacttcagaacataatgcgccatgttcttcccaattttgacggtatatatgctggccagacaatggatagaatcaagtcgatgagcttactgcgaattgctactcaagatgcaattcacaggctcgttagtcaagaatccgtggagatcaatatagatttggggaatggccacatggtgtccaaagttctagaccattcagagtttgaacaagtcaaccgggcaattttcgacaagtgcgaaaagatcatcaaccagtgcttggctgatgcgaagttggtaccagaggacatcaatgatgtcatactggtcggaggttgttcgaagatccccagaatcagaagccttgtcctgggtttgtgcaagaaggaggtctcctacaagaacatagatgctctagaagctgctgtttcaggtgctgcgctggaaggagccatcgcttcgggagtaaatgatccttcggggagcttggatcttctcacgatccaggcaactcccatgaacctgggaatccgtgccgacggtgatagcttcgctgcgatcatccctaggaacacgacggttccagcaaggagggacatgctgttcacaacgacacacgacaagcagaccgaagccctaattgctgtgtatgaaggagaaggggaacgtgctgaagataaccatctgttagggtacttcaagatcactggaattccggcagcccctaagggagcagttgatatcagtgtatgcatggacatcgatgctgccaatgtcctcagggtgttcgcaggggtcgtgaagcctcaaggcccagccattccaccgttcatcgaggtgaggatgccaacgctggacgatggccatggctggtgtgggcaagctctagcaaagatgtatgggaagactctcgaccttgctgttcttccgaagaagctgcagccatga

SEQ ID NO: 27 - DNA, 423 bp, Zea mays
atgggcaagtcctggtcgctcatcagccacctccacaccgtcgccgggccaagcatcaccctgctgtaccctctgtacgcgtcggtgtgcgcgatggagagcccgtccaaggcggacgacgagcagtggctgtcatactggatcatacactccttcgtcaccctcctggagatgctcgccgagccgctcctccactgggtacccgtgtggtacccggccaaggtgctgttcgcggcgtggctggcgctgccgcagttcaagggcgcctccttcgtctacgagaagctcgtcagggaacagctccggaactaccgcgccagatacccgcgcaagggcgccgccgccgccgccgtcggcggcgacgacgacgaccataaggtgcacatagccaaggctgaggctgagcacgaccatgtgcagtga

SEQ ID NO: 28 - DNA, 1320 bp, Zea mays
atgggcatctcggcgaggtggctcaagtcgttagttgggttgaggaaggtggggcggcagcagcagcagcggcgaaaggatgatgcagatgttggtcgaatgaaaaccgatgtcgccgaccagtttcattttcagattcagcactctcaagatgacaatagcattgctgcacaagaaattccagaggtttcttatggaaatgatccacctgaagatgattctaatgtaccttcatgcttcgagcctgctcgtagttcagctcatatgccattttgtcaaactgaagaagcccagaaagagatctgggctgctacaattattcagacagcatttagagctttcctggcaaggagagcccgccgagctttaaaagggttggttaggcttcaagcccttgtaagaggtcatatagtaagaaagcgagcagctacgacactccgttgtatgcaagctttggtcagggttcaggcccgtgttagagcaaggcgagttcgcatggctttggaaaaccagactgatcggcaaaatacttcaccagagcacacgatcgaggcacgtgtcagagaaatcgaggatggctggtgtgatagtatcggttctgtgggagatatccaagcaaaacttctaaagaggcaggaagcggcagctaaacgtgagcgagccatggcctatgctctagctcatcagtggcaggcaagttcaagacaacctacagcatttgaacctgacaagaacagctggggctggaattggctagagagatggatggctgttcggccttgggagagtcggttccttggcacttacgggacagatgggatttttgtagttaatgaaaccagacaacctgacagaagtgcaaccaagaccccatacagaaaacctgttaaaaagcatgattcaacccttcaatcaaacatgttgaaccagaaggtcttcccatcaaactcagagggtggtggctcctcgacaaatcgatcaagtggtttggtatcaactaaatctagactgaaggttttacccagagaaggttctgaggaagcctcatctcatccttcagtaattgtgcggtccactagtaatccgaaggagaggatttccagtattaattcaaaggagaggactggggacttggattttcaagttcataaaagatcctccttgcctagcattggtccagaagctggcaaacacctgacaaagaaaggcatggttaaccgatccctgaaggccacaaaagattcccaaatactggggtcaaggcatcatcttgccagttccattgatccagttcccaccagagttgaactggagacttga

SEQ ID NO: 29 - DNA, 498 bp, Zea mays
atgcacggccaccagcagcagccgtacccatacgggctgcggcaagacccgctgcggcggatgcgaggtctggacgtgaaatcggcgctccaggccagcccgcgcacgtccacggtggccacggccgcactcgtcgtcccgctgggtgccgcgctgctgtgcgcgtcggggatcgcgctggctgcgaccgtgaccggtctcgcgctcgccacgcctcccctcgtgattttcagcccggtgctcatacccttcgcgctggccgcggggctcatcgcgtcggggctcctcgcgtcgggcgcgctcggcgtcgcgggcgtctcggcgctcacgtgggccgtcgggtacgtctggcaacggcagggcgaaggcggcggcggggtcactgggatggtggtgcagccgctggaccaggggtcgaagcgccatggtgtccaaggcgccgccgcgttcgtggggcaccgccgcctgccacgggacatcgacgttgctggtgaatga

SEQ ID NO: 30 - DNA, 1569 bp, Zea mays
atggatcgcctcactgtgatggcgccgccgctgctcgtcctcctcctgctgctcctctcgcggtgctcggcggcggcgtcacggcgcggcgggggctggtgggaggagggcgagggggagtggaggccgagcgaggaggaagagaagggcaaaggcaaagggagggggctgttcctgctgcaccgggtggagaaggtggtggagtcggagggcgggcaggtgcgcgtggtgcgcggccagccgtggccgccggcgtccttcgcctgccgcgaggggctcatgcacatcggcttcatcaccatggagcccaagacgctgttcgtgccgcagtacctcgactccagcatcactctcttcgtgcagcggggggaagcgaaggttgggtatattcacaaggacgagctcgtggagaggaagctcaagatgggcgacgtcctccacatcgacgccggctccaccttctacatggtcaaccccggcaaaggccagaggctgcagatcatatgcagcgtggacgcctccgatagccttggctttggacctccttatcaggccttcttcctcggcggcgcaggggacccggcgtcggtcattgccggttttgggccgaagacgcttacccgtgccttcaacgccacctacgacgagttggcgcgtatcctgttgccacgaaccggcggccccatcgtgtactacaccgcggacgcggagccagagagcggtgccgccgaagaggagcgcgggcaggtcgacgggcacgacggtgtgctggacaggggagcgcgacgcgagggcgccggcgcgtgggtgccgggtggcaggggagacggaggcgacgagtgcggcggcagcgatgacgcgcgtgaggcgacgtggtggtggacgaagctggttaacagggtcgtcggaggggcggctggcggcggcggcgctgcggaggcgaacaggaagggcaagaagaagaagggcggcgcgccggagccgtacaacctctacgacagcgagcccgggttccggaacgcgtacggctggaccgtctccgtcgacaagcaccagtacgagcccctcaagcacccggacatcggcgtctacctcgtgaacctcaccgcggggtcgatgctggcgccgcacgtgaaccctcgggcgacggagtacggcgtggtgctgggcggggaaggcacggtccaggtggtgttcccgaacgggtccctggcgatgagcgaggtggtgcgccccggcgacgtgttctggatcccgcgctacttccccttctgccaggtggcggcgcgggccgggcccttcgagttcttcggcttcaccacctcggcgcgccgcaaccggccgcagttcctggtcggcgcctcctcggtgctccgtaccatgctagggccggagatcgccgccgcgttcggcgctcgcgagaaggagttcagtaagctggtgcgtgcgcaacgggaggccctgattatgccgtcctctcctgggaaggaggaggaggagcatgggaagaaagggagggagaaagaggagtcactgccgatggttgtcgagcaggcggcggcggagtga

SEQ ID NO: 31 - DNA, 1644 bp, Zea mays
atggcgatggcctccgcggcttgctcatgcacggacggcacgtggtgggtgtacgcgctcccggcgctgctcggctccgacaccctgtgcgcccacccggccctcctggctggcctgatctttctggccaccgtctcggtggctctgctggcgtgggccacgtcgccgggcggtccggcgtggacgaacggccgcggcgcctcggcgtcactcctatcgtgggaccccgtggtctgcccgtgttcggcagcatcttcgcgctgtcccggggctgccgcaccgcgccctcgccgagatggcccgcgccgcaggccccgggccaaggagctcatggcgttctccgtcggtgacacgcccgcggtcgtgtcgtcctgcccggccacggcacgtgaggtgctcgcgcacccgtcattcgccgaccgccctgtgaagcggtcggcccgggagctcatgttcgcgcgtgccatcgggttcgcgcccaacggcgagtactggcgccgcctccgccgcgtcgcgtccacgcacctattctccccgcgccgggtcgcctcgcacgagccgggacgccaaggtgacgcggaggccatgctccgctccatcgccgccgaacagtcggcctctggcgccgtcgccctccgcccgcacctccaggccgccgctctcaacaacatcatgggcagcgtcttcggcacgcggtacgacgtcacatcaggcgccggcgccgcggaggccgagcatctcaagagcatggtgcgcgaggggttcgagctcctcggcgccttcaactggtccgaccacctcccctggctcgcccacctgtacgacccaagcaacgtcacccgccggtgcgccgcgctcgtgccgcgcgtccagaccttcgtccgtggcgtcatcgacgagcaccggcgccgccgccaaaactccgccgccctcaacgacaatgctgacttcgtcgacgtgctcctctccctcgagggtgacgagaagctcggcgacgacgacatggtcgccatcctctgggagatggtcttccgcggtacggacacgacggcgcttctgaccgagtggtgcatggcggagctggtgcgccacccggcggtgcaggcgagggtgcgcgccgaggtcgacgcggctgtcggtgccggaggttgccccaccgacgccgacgtggcgcgcatgccgtacctgcaggcggttgtgaaggagacgctgcgcgcccacccgcctggcccgctgctgagctgggctcgcctcgccaccgccgacgtgccactctgcaacggcatggtggtcccggctggcaccacggcgatggtgaatatgtgggccataacccacgatgccgccgtgtgggccgacccggacgcgttcgcgccggagcggttcctgccctccgagggcggcgccgacgtggacgtccgcggcgtcgacctccgcctggccccgttcggcgccgggcgtcgcgtctgccccggcaagaacctgggcctcaccaccgtgggcctctgggttgcccgcctcgtgcacgccttccagtgggccctgcctgacggcgcggcggccgtttgcctcgacgaggtcctcaagctctccctggagatgaagacgccgctcgtcgccgcagccatcccccgcaccgcctga

SEQ ID NO: 32 - DNA, 1488 bp, Zea mays
atgtcgtacaacaaggccccgtccatcaccgccgagaccataaatcccagggtcaagatcatcagatacgcgccttgcggggagatcgtcaagcacgcagcgcggctggagcaggagatagaggagaaccctgcttctatccctttccaagagataatatactgcaacctcgggaacccccaggtccttggtcagccgccactaactttctttcgtgaggttctctccctgtgcgacaacccagccctcatggacagagatgaagctcgtgccttattcagcccttgtagcataagaagggccaggaggatcctaaactcgatccccagcaaagacactggtggatatactgattgccggggaatcaaatgcctgcgtcaagtagtagcagatgggattaccgcaagagacggttttccatcgacagcggacgatatctttctgacagatggagccacctcagcgatcaacttgatgatgcagatactcatcaggtctcacgaagacggcatcttgagccctctacccgaataccccttgtactcggcctccatcatactgcacggtggaacgatggtgccgtacaatctcactgaagataacggttggggcctagagatctttgaagtcaagaggtgcttggaagaggctcgttcggctggcctcactgttagagctatggtggtaataaatcccggcaaccctactggacaggtgctatccgtcacaaaccaggaggagatagtagagttttgtcggaaggaaggtctggttatacttgctgatgaggtataccaggaaaatatctacgcagagaacaagaagttccattccttcaagaaagtggccagatcacttgcttacgacgaaaatgacctcaccctagtatcatttctttccgtttcaatgagctatggggagagtggcagaagaggaggctacatggaggtgtctggtgttgcagctaatgtgaaggatcagatttacaaggtggcttctttaacactctgccccaacattgctggccagattctcgtcagccttgcaatggatccaccgaagctaggagatgaatctttcgagtcatttgataaggattttgttcagatccgttcatccttctgcaagcgagccaagaccctggagaaagctttcagcggcctggagggcgtgacttgcaacaagttagagggtgcgctgtacttgttcccacggctccacctcccctccgctgcgatcagggccgccgatttcgagggagtttcacctgacatattctacgctcaccgcctgcttgatgccaccgggattgccgttgtgccgggctctgggttccaccaggcctctgggacgatccatatccgatgcacgatccttcctgacgaaagaaaaatcgaggcgatggtcgcacgcttgagggcgttccataaggcgttcatgaacgagttccggggctctcagcaggtcatgaacgatctccgtcgctga

SEQ ID NO: 33 - DNA, 561 bp, Zea mays
atggcggaccgtgaccgcagcggcatctacggcggcgcccacgccacctacgggcagcagcagcagcagggaggaggcgggcgcccgatgggtgagcaggtgaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcaccgcaggatggcggaggccgcggcgcacgcgggccacaagaccgcgcaggcaggccaggccatccagggcagggcgcaggaggccggcgccgggggaggtgcaggtgccggcgctggcggcggcggcagggcttcctcgtaa

SEQ ID NO: 34 - DNA, 531 bp, Zea mays
atggcagatcgcgaccgcagcggcatctacggcggcggcgcctacgggcagcagcaggggggcggccgcccgatgggtgagcaggtgaagggcatgatccacgacaaggggccgacggcgtcccaggcgctgacggtggcgacgctgttccctctgggcgggctgctcctggtgctgtcgggcctggcgctggcggcctccacggtggggctggccctggccacgcccgtgttcctgctcttcagccccgtgctcgtccccgccgcgctgctcatcggcacggccgtcgcggggttcctcacgtcgggcgcgctggggctcggcggcctgtcctcgctcacgtgcctcgccaacacggcgcggcaggcgttccagcgcaccccggactacgtcgaggaggcgcgccgcaggatggcggaggccgcggcgcacgcgggccacaagaccgcgcaggccgggcacggcatccagagcaaggcgcaggaggccggtgctggcactggcgccggcggcggcaggacttcctcgtaa

SEQ ID NO: 35 - DNA, 474 bp, Zea mays
atgggagaccggcatcagcatgggcctggcagcggcgtggaggacccggcgacgctgctgcggcgggtgcagacccgcgcgcccaactcgacgcaggtggtggggttcctgacgctgctcgtctctggcgccgtgctgctgctgctgacgggggtgacgctgacgggcgccgtggtggcgctcgtcttcctgggccccatcgcgctgctcaccagccccatctgggtgcccgtcgccgtcgtgatctccgccatcgtcgccgccgcgctctccgcctgcgccttcgccgccgccgcgctccccgtggccacctggatgtaccgctacttcacgggccgccaccccgtgggcgccgaccgggtggactacgcgcgcagccggatcgccgacaccgccagccacgtcaaggactacgcgcgcgagtacggcggatacctgcgcacccgcgccaaggacgccgcgcccggcgcgtag

SEQ ID NO: 36 - DNA, 480 bp, Zea mays
atggacaggaagtgcctcgtggacctcggccacccgctgctcaaccgcgtcgccgacagcttcatccgcgccgctggggtcggggcggccagggccgtctccagggaggcctacgtcgtcaccgtcgaagggctttcaggagactcgtccgggctagacgccgacggcggcaagcggagccatttctccagcatcagaggtgacgacggccagaggtcgctcgacgcagtggtaaaaacggccggcaaggaggccttccagtgggggttggcagccggagtctactcagggctcacgtacgcgctgcgggaggccaggggatgccacgactggaagaacagcgccatcgcaggcgccatcgctggggcggcggtggcgctcacgggtgacgccggcggccactccgacaagctcgtcaacttcgccatcaccggcgccgcgctctccagcgccgggagcttgctctccggcatattctga

SEQ ID NO: 37 - protein, 151 aa, Zea mays
MVTVEGRRGSSDQTGDMRYVSDFPVYDGRGSDAVLVARVQGVTTTFGNSNQFFTVAFEAGSRLKGSTLLTEGVVTDGSDEWAIYGGTGEFAMARGVVKRRYLADRDGAGNTDELSMQVFCPVFGSSQQPNKQDSSISVTKIGLWGADFDLI

SEQ ID NO: 38 - protein, 163 aa, Zea mays
MARFVDPLVVGRVIGEVVDLFVPGSKDISNGCLLKPSATAAPPLVRISGRRNDLYTLIMTDPDAPSPSNPTMREYLHWIVINIPGGTDATKGEEVVEYMGPRPPVGIHRYVLVLFEQKTRVHAEAPGDRANFKTRAFAAAHELGLPTAVVYFNAQKEPASRRR

SEQ ID NO: 39 - protein, 425 aa, Zea mays
MAQLRAAADEQQEAAMHGVASGDGNESNKKARAGLCGLLRERKVVDLARAKRRLVEVPYTATLAHTANALLAARVSAVAVAAPPGHWIGAGGSMILESDPATGAVRKHYIGMVNMLDILAHIAEASDDDEAADLDRRMAVPVSSVIGHSLEGLTLWTLHPSTSVVDCMETFSKGVHRALVPLESSADNVVALELVESAPGYRMLTQMDVVRFLRAHGAELRGVLLRTVRELGAVNDTVFAVSGVTKVIDVIKAMRAASLTAVPVVDAVGSGTETLQYGSGQRAVETFSATDLRDCPVARLQAWLGISVMEFKRKVAEYRASTRLVVPGADATDTGTPVAGYADTPAAAAATDEEQSEQQESALVTCSQESTLGEAIEAATSRHVHRLWVVDEEGLLRGVVSLTDILRAVREAALGEDLELHSIVP

SEQ ID NO: 40 - protein, 324 aa, Zea mays
MAENPAPSSPLVTSPDAAAPPLPAEFTKLTFRSLYIRRTGPGSREMTVDGRPSDQLGRRFVSDFPIYDGRGSGARLVARLQGVTVQIGSSHQLVSIVFEAERLKGSTLLTNGVITDGSDEWAIYGGTGVFAMATGVIRRRFLAGSNDGNSDELTSIEVFCPAFGSAQQQPNKQDSVSVTKIGVWGGGGGSAQDITTTEPPQRLHSITVRTGFAVDSIEFTYTDRGGQRRTAGRWGGLGGNLRTIDLGDAEVVREVSGTYGTFEGATTLTSIRILTSSRTWGPWGIEDGTRFCITAPIGSSIVGFYGRSTSRLVAAIGVYLRQQL

SEQ ID NO: 41 - protein, 190 aa, Zea mays
MAPKRRGNKVVGSVVKTKLVQETVEVIVADDDGLHAEKQQVPEALALAHPTVDVSGSTVVHVVEVTAKRGRGGGGGEESSNTNKEDEDAAAAAAAAAAGAGQLRRRRRDGRRGRVQAVRVARAEAGAPGPGRVGARHAGAGHDDGRHVRAPRGGGGAPVQGHGQGDAHLPRGAERRQARAPRGAGQARHL

SEQ ID NO: 42 - protein, 116 aa, Zea mays
MPSTVNSASLDQNLELAKQCSREAALAGFKAAAVATVASAVPTLVSVRMLPWAKANINPAGQALIVSTVAGMAYFIAADKKILSLARQHSYENAPEHLKDTSFQGTGRPHPGFFRP

SEQ ID NO: 43 - protein, 129 aa, Zea mays
MAHSKREIKPGRAHTKVAGDDEMLRTGFFDGTPLEGGKIADSQPVDLFAAARRVADAPYSSQQEGPHEEGTNKNAVIAESEPVDLPASARGVAEVNRAGPEEQTGRGRQGMTEPTAGGRRLGLGRPAPA

SEQ ID NO: 44 - protein, 578 aa, Zea mays
MAEQFYTVASDSETTGEDKSQASFPDVAVGIDIGTSKCSVAIWNGHQVELLKNTRNQKGMRSYVMFKDDTLSAGVTGGAAKENAHEERDILSGSAIFNMKRLIGRMDTDEVVQASKTLPFLVQTLGIGVRPFIAALVNNMWRSTTPEEVLAIFLLELKALVEMHLKHPVRNAVLTIPVAFSRFQQTRIERACAMAGLHVLRLMPEPTAVALLYAQQQQQLVHDNMGSGIEKIALIFNMGAGYCDVAVAATAGGVSQIRALSGCTAGGEDILQNIMRHVLPNFDGIYAGQTMDRIKSMSLLRIATQDAIHRLVSQESVEINIDLGNGHMVSKVLDHSEFEQVNRAIFDKCEKIINQCLADAKLVPEDINDVILVGGCSKIPRIRSLVLGLCKKEVSYKNIDALEAAVSGAALEGAIASGVNDPSGSLDLLTIQATPMNLGIRADGDSFAAIIPRNTTVPARRDMLFTTTHDKQTEALIAVYEGEGERAEDNHLLGYFKITGIPAAPKGAVDISVCMDIDAANVLRVFAGVVKPQGPAIPPFIEVRMPTLDDGHGWCGQALAKMYGRILDLAVLPKKLQP

SEQ ID NO: 45 - protein, 140 aa, Zea mays
MGKSWSLISHLHTVAGPSITLLYPLYASVCAMESPSKADDEQWLSYWIIHSFVTLLEMLAEPLLHWVPVWYPAKVLFAAWLALPQFKGASFVYEKLVREQLRNYRARYPRKGAAAAAVGGDDDDHKVHIAKAEAEHDHVQ

SEQ ID NO: 46 - protein, 439 aa, Zea mays
MGISARWLKSLVGLRKVGRQQQQRRKDDADVGRMKTDVADQFHFQIQHSQDDNSIAAQEIPEVSYGNDPPEDDSNVPSCFEPARSSAHMPFCQTEEAQKEIWAATIIQTAFRAFLARRARRALKGLVRLQALVRGHIVRKRAATTLRCMQALVRVQARVRARRVRMALENQTDRQNTSPEHTIEARVREIEDGWCDSIGSVGDIQAKLLKRQEAAAKRERAMAYALAHQWQASSRQPTAFEPDKNSWGWNWLERWMAVRPWESRFLGTYGTDGIFVVNETRQPDRSATKTPYRKPVKKHDSTLQSNMLNQKVFPSNSEGGGSSTNRSSGLVSTKSRLKVLPREGSEEASSHPSVIVRSTSNPKERISSINSKERTGDLDFQVHKRSSLPSIGPEAGKHLTKKGMVNRSLKATKDSQILGSRHHLASSIDPVPTRVELET

SEQ ID NO: 47 - protein, 165 aa, Zea mays
MHGHQQQPYPYGLRQDPLRRMRGLDVKSALQASPRTSTVATAALVVPLGAALLCASGIALAATVTGLALATPPLVIFSPVLIPFALAAGLIASGLLASGALGVAGVSALTWAVGYVWQRQGEGGGGVTGMVVQPLDQGSKRHGVQGAAAFVGHRRLPRDIDVAGE

SEQ ID NO: 48 - protein, 522 aa, Zea mays
MDRLTVMAPPLLVLLLLLLSRCSAAASRRGGGWWEEGEGEWRPSEEEEKGKGKGRGLFLLHRVEKVVESEGGQVRVVRGQPWPPASFACREGLMHIGFITMEPKTLFVPQYLDSSITLFVQRGEAKVGYIHKDELVERKLKMGDVLHIDAGSTFYMVNPGKGQRLQIICSVDASDSLGFGPPYQAFFLGGAGDPASVIAGFGPKTLTRAFNATYDELARILLPRTGGPIVYYTADAEPESGAAEEERGQVDGHDGVLDRGARREGAGAWVPGGRGDGGDECGGSDDAREATWWWTKLVNRVVGGAAGGGGAAEANRKGKKKKGGAPEPYNLYDSEPGFRNAYGWTVSVDKHQYEPLKHPDIGVYLVNLTAGSMLAPHVNPRATEYGVVLGGEGTVQVVFPNGSLAMSEVVRPGDVFWIPRYFPFCQVAARAGPFEFFGFTTSARRNRPQFLVGASSVLRTMLGPEIAAAFGAREKEFSKLVRAQREALIMPSSPGKEEEEHGKKGREKEESLPMVVEQAAAE

SEQ ID NO: 49 - protein, 547 aa, Zea mays
MAMASAACSCTDGTWWVYALPALLGSDTLCAHPALLAGLIFLATVSVALLAWATSPGGPAWTNGRGASASLLSWDPVVCPCSAASSRCPGAAAPRPRRDGPRRRPRAKELMAFSVGDTPAVVSSCPATAREVLAHPSFADRPVKRSARELMFARAIGFAPNGEYWRRLRRVASTHLFSPRRVASHEPGRQGDAEAMLRSIAAEQSASGAVALRPHLQAAALNNIMGSVFGTRYDVTSGAGAAEAEHLKSMVREGFELLGAFNWSDHLPWLAHLYDPSNVTRRCAALVPRVQTFVRGVIDEHRRRRQNSAALNDNADFVDVLLSLEGDEKLGDDDMVAILWEMVFRGTDTTALLTEWCMAELVRHPAVQARVRAEVDAAVGAGGCPTDADVARMPYLQAVVKETLRAHPPGPLLSWARLATADVPLCNGMVVPAGTTAMVNMWAITHDAAVWADPDAFAPERFLPSEGGADVDVRGVDLRLAPFGAGRRVCPGKNLGLTTVGLWVARLVHAFQWALPDGAAAVCLDEVLKLSLEMKTPLVAAAIPRTA

SEQ ID NO: 50 - protein, 495 aa, Zea mays
MSYNKAPSITAETINPRVKIIRYAPCGEIVKHAARLEQEIEENPASIPFQEIIYCNLGNPQVLGQPPLTFFREVLSLCDNPALMDRDEARALFSPCSIRRARRILNSIPSKDTGGYTDCRGIKCLRQVVADGITARDGFPSTADDIFLTDGATSAINLMMQILIRSHEDGILSPLPEYPLYSASIILHGGTMVPYNLTEDNGWGLEIFEVKRCLEEARSAGLTVRAMVVINPGNPTGQVLSVTNQEEIVEFCRKEGLVILADEVYQENIYAENKKFHSFKKVARSLAYDENDLTLVSFLSVSMSYGESGRRGGYMEVSGVAANVKDQIYKVASLTLCPNIAGQILVSLAMDPPKLGDESFESFDKDFVQIRSSFCKRAKTLEKAFSGLEGVTCNKLEGALYLFPRLHLPSAAIRAADFEGVSPDIFYAHRLLDATGIAVVPGSGFHQASGTIHIRCTILPDERKIEAMVARLRAFHKAFMNEFRGSQQVMNDLRR

SEQ ID NO: 51 - protein, 186 aa, Zea mays
MADRDRSGIYGGAHATYGQQQQQGGGGRPMGEQVKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEAHRRMAEAAAHAGHKTAQAGQAIQGRAQEAGAGGGAGAGAGGGGRASS

SEQ ID NO: 52 - protein, 176 aa, Zea mays
MADRDRSGIYGGGAYGQQQGGGRPMGEQVKGMIHDKGPTASQALTVATLFPLGGLLLVLSGLALAASTVGLALATPVFLLFSPVLVPAALLIGTAVAGFLTSGALGLGGLSSLTCLANTARQAFQRTPDYVEEARRRMAEAAAHAGHKTAQAGHGIQSKAQEAGAGTGAGGGRTSS

SEQ ID NO: 53 - protein, 157 aa, Zea mays
MGDRHQHGPGSGVEDPATLLRRVQTRAPNSTQVVGFLTLLVSGAVLLLLTGVTLTGAVVALVFLGPIALLTSPIWVPVAVVISAIVAAALSACAFAAAALPVATWMYRYFTGRHPVGADRVDYARSRIADTASHVKDYAREYGGYLRTRAKDAAPGA

SEQ ID NO: 54 - protein, 159 aa, Zea mays
MDRKCLVDLGHPLLNRVADSFIRAAGVGAARAVSREAYVVTVEGLSGDSSGLDADGGKRSHFSSIRGDDGQRSLDAVVKTAGKEAFQWGLAAGVYSGLTYALREARGCHDWKNSAIAGAIAGAAVALTGDAGGHSDKLVNFAITGAALSSAGSLLSGIF

SEQ ID NO: 55 - DNA, 4178 bp, Artificial
  Vector
ttggttttttgataatttgtttatctcacatgatggttctaacaatatgcgcagatcttatacatctctctcgtagtttttcataaactacgagaaagatataggttttatgaacaaatttacttttattttgtcatataatgaaatggtcaaaatataaattgtacatcatgatgagttatacaaatttgtagtccaaaatcttttcatttaaattaatttactgctttaaaatgtgatttttaattgtctttgcctagtgttgaaaaaaagcactcggcaaagaaactctttgccgagtgttaaaaaaacactcggcaaagaatctctttgccgagtgtcaaaaataaaatatttggcaaatagcttctttgctgagtgtttttttgcttagcacttggtaaagaacttctttgccgagtgtccgaaaaagcactcgataaaatatttggcactcagcaaagaaccgaattccagtagcgtgagtcagaagttggcatttgaaccctttgaaataatcaaacaaatcaacgcagttgcatatgaattggctttgccacatggtagtgcagtccatcgagtgttccatgtttcacagcttaaactaacccatggttttggtaacagaacacacaccagtgactccatcgttaccggattttgatactacactgaaggttccggaacaaatactggattctagattgattctagattgcttcgatcaaatggggatcatcaacctaacacagcttaaattatacatgcatgcgtacatttggtccttcaagaggaggcaggtgatgatcaaggtctctctagcggcctggtgtgcagccacgtacccatccacgcgtcgccgcgggcgacgacaggtgttggccaacctggacatcagacaagcacgacaatgacaggcgttcgccaagcgtacgtacaggagacgaacacgacaagtagcaccgtcctggccgagagcccgtcatgctcatgcatgcatgcatgctgatcatgcatactcgataaatatacagcagagagctgagctacgtacacaaacaagaagcttgcattgttcgttgctcgatcgttgcaggtaatggagaaccctgctccgagtatcgtagtgccccccatcgccgcacctgtatcagcgaacttcagcagactcgccttccgcaacctgtacatccgacggacgggtccagacagcagggagcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 56 - DNA, 3882 bp, Artificial
  Vector
ttagtagagaataacacacatctccctaagtatcacaaaaaaattaattttaagaggcaattttagtttccaaagcgatttattaagatttaagggagggtatttattagaaggaagtacattgatcaaacagagaaaattccatcattatggtcccccatagaaatttgtcactcgattgtcgcaaaaaaagttaccagtaaatttgataaattttggataccggtctgttttaactcatcaagaatagcacgacgaaataatagattgcgtacgttttcattcataacatctctcactaaagcattttttacacacgctattaaaatggttcggaaactgatccttaagcggtctattcatatccatctatcgttctaagattcttcctgtacgtagattctttccgattaaattctcatgaaaaattaaaaacaaacaggtccttaaactagtcgccggaagttctcgcttttgctgtagttgtagtgtcgatgcatgggtggaaacgtggacagatagattcatgaaatgtggacaacccacctggtccagagcatcatgatacattgatacacgtggcacgctgcccactgatgccctcagctttgaagctaacgtgtcaagtgtggccggcccctgcattgcgtcgtgccaatcgtccaagtcccatggcaaaaacccagcgctttgtgccgccgccgtccgccggcccctctgcccttgtacgtgcacctagacacatcgtcatcgatcatcacacgcaatcgacacaagaagttaataaacagcccaaggacgcagagatcagctgatcgagaaggacttgtactactactcagtattgtcgtcacatgcacatatatgtacataaagagctagctacctgagctctacccaaggtcgcgttgatcgatcgatcacgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 57 - DNA, 4949 bp, Artificial
  Vector
gattcagaacatctggtcagcttaaatgatgtggtaaagggagcattattcttttattttgttctttgttctgtgcaatggccatatattttcgtattgatatgctctcagtggaacagtctgaagctaatgtgaaacttcgatgttcaggtatgataatgtaccaacagagattaaccggctgagatgcagagttaactatcatgcattaaagttcctacctgatattgaggaaatggctgttaagcttgctgcaagaatgaggaacaaaactggcagcataaatccatacatgtacgttataattatccttttcgctgggtgatattgtgtacagtagagcttgtatctacccaagacactatccagtgcatagctgaagttgacgctctcaaagtttatttcacaatcaaatattgacacattttttttgttcttaaagggctcttcacctgagatttgaaaaagggatggtggggctttctttctgtgattttgccggcacacgggaggagaaggcaatgatggcggcttataggcagaaagaatggccaaggcgcttcaaggtgacgcggactggactagatacatctgttacattggactgagaaagaatccctaggagaaatgaagcaccgacactaactatacgcgctagttcgggaaccccgttttctcacgagacttttatttttccaagaaaaattagttcattttttttaggaaaatataaatctcttaagaaaatgtagttccgaaactagccctacaagtctaacgattttctcttttttgtatctgccacgcagaatggatcccatctatggccactggcgctgcagaagagaaaagaagggcggtgccctcttgagcccggtgagatcgctgtgatcctgcgggcactggggtacacgagcggcacacagatatacgtcgcgtccgggcaagtgtacggcggcaagaaccggatggctcccctcaggaacatgttccccaacctggtaggcaggctcaaatccacttcagattgtgtcaagctgaagccagtgataactgataagccactgccgtgcattgtgcatcgctgcaggtgacgaaggaggagctagcgagcgcggaggagctggcgccgttccggcggcacgtgacgagcctggcggcgctggacttcctggtgtgcctgcggtcggacgcgttcgtgatgacgcacggcggcaacttcgccaaactcatcatcggggcgcgccgctacgcggggcaccgcctcaagtcggtgaagcccgacaagggcctcatgtccaagtccctgggcgaccccgacatgggctgggcctccttcacggaggacgtcgtcgtcacgcaccgcacgaggacgggcctccccgagcccaccttccccggctacgatctctgggagaacccgctgacaccctgcatgtgcagggcatgagacgggccatccctcagatcagagtggccgtctggccgagtcctcgtcttcgctaggctacgactacgagtagtctcgtgcccacttccatcagcgcgcatttcagcgagcatatgcttctactagttaaacacctagatttgtatgtatttggctggcctaatctgtagtttcacatagatcacttcatgctgaaaatgtcttggacaaggcagtgaatgaagataacatcagtaacgacctgcttttccaaagctcctcttggatttaccaagctgagccaagaaatggctaacctgagttttgacttgcagccttgcaggcatgacgtgggcgacctgaactcgctagtcgtagagtttctttccacgcggcagctcggcagagctacgcagcctctctgcccgccacgttctcctgcaaggctgcaactcgaagcactgagaagctattaattaatggattctcgagcatgcggccttgttcgctaaacctcgcgcacagaaacacaagcgagtaccagagcagctaggagtggtcacgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 58 - DNA, 3638 bp, Artificial
  Vector
gccagtgctaatgatatttacactagcgggctgctaaagaaaaccgccagtgctaaagatatttacactagcggttggtgaacaactgcctgtgaaaaaagccgatttctactagcccctagctagcactggcgacataaaaaacgtcagtgaaaatagctctaggatcgtcactatagagcttctatgtacttagtggttagaactgatattgtagtgcaccaagtgccgattttaattaaaccaatactaaatactagtaaataatactagtggtctgaattcgatttctatagtaatgtttgcttgcaagccgcaaatagagtaaacattcgtcgtcacagaaatccacattacatcaaggtccatggcggccggccacgtacccatcccacgcgtcgctgcggaggacacgtgttggctgaccggacagttggccgatcagacagtggacagaccggacaatagaagaagaagacgacgacggcggcggcaccgccgagtaggtgcatggtcacgctagctgtagctttttgcagagcgtcgtctgtaaatacgtagcccttccacaagcgaggcaaggggggagagagtatcgtcagctagcagagagagtgcgtagcaactagcaatggcggagaaccctgctccatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 59 - DNA, 3657 bp, Artificial
  Vector
gtgattaagttgactggcaaattgcatagacgataatacactctatgcttttgagataaccactgtacaccaaccgacttgttgtttgaaggctgatctttaaccattactactgtaactaactgttgttgaataaactattcatacgacttggagactttgatgtatattatgttttgtacctatactattaactaccgcagatacgtatttacatctcggtaacatctgccagtcgcacaaatgcactaaggtgactgacagactgtagcgaccgatagagcgctgccttgttctagtcaaaattcaagcctggtggtcttctcgatccctctgcgcgcgccctgctcttttatcctgtgtgtctcctccacgagggaaggcgtcatgagaggacgacacgacgaccatgcagtgctgcgcaaccgcgcgcgtctgcctgttcactgagccccaggcggccggtccaagctattaccacgtcgcccccctccagggctgcgggcagcaacgtgtcgaccggtccgcctctgacgtgtccctcccgtcctctgcataaaagggtgcggctggcggggagcctcagcttcatatcgtcgcaagctctccgtgtttctctcgtttcttcactgctgccaactgtgctgtgcagtagttgcaagagctgattggtagctaggtacgctaggcaagtaggccgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 60 - DNA, 4315 bp, Artificial
  Vector
tataaattagaacggagggtatgtttttttacgtttccattatttgagtctgctcatcacgtttaatttgtttatacagttcaatgagttcagttacagcggatgataattcgacggcgtttatttgcatgcacttactttttacattacgcctagaatgtaatcggatggacgaatattacttgttttgtgtttattttcatatttttttctcttcggatacggatattatcgagttgtgccgaacaagattttgaatgtatatcgacatcttaaatatataactttgaatatacggatacgtatatagatcggatattgaatacccagctagactcagattaaattggatcttagttattttagggggtgtttgaatgcactaaaactaatagttagtggctaaaattagttgaaacatccaaacaccctatctaatagttcagctattagttatttttggaaaattagttaatagttaggtagttatctgttagctagctaattcaactaattataagttctagtgcatttaaataccccttagacggatcgaattagaacaccgatggataatatccataacattttacacctataaatatgtcctatatgtatatagatgtcaaaaacaggttgggcatgttggatgacccgtggcacgatacagttttaagcagtgccaaacatggcatggcggcgagctagacatgatacggtccggttttatttttgtatttttttttcgtaataatatctgtattatgcttgttcgaacctcacttgcttatgtggatgtgatagtatcaactcgctggcgttccgtgcatcgtaacatctatcacatcccacagatcatgtcgcttatcttttctgatactatagcagtagcacacagatgccggtcgaacccagaagcatcacgccatcaccccctccccttttcccaatcaaaaccacgtccgtcccaatggtaacccagcaccaatctcgtgttcgtgtgagacagagcatgcaaacaaacagcatgcatgcgcgaaggaaagcgtgcagcgcgcgggacgtgaaccgcgcgcgaaccctgccgcccgtccggtctcagctcacgcccagcgtccttcccaggccgcgatccggccatcctgacgcgcgtttcgtctgggccccaaaaccacgtccgccgcggcggcggcgcgcccacgggctcccgtgcccgccgatagtttgccggaccctgccgcctcctataaatgcagagccctccgccagcgtctcccgcatctcatcttcttcctccatccgcactactgagagcgacaaagctagctctctccagggatcggattcggatcaccacgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 61 - DNA, 4960 bp, Artificial
  Vector
aagggactcgtggcctacacacgcgcacaataatcagttaccatgaattattttatatgcgaatatcgacattacacatgaataatgtgaatttgaatttccgcagcatgcaaaattcccgtgagtttgaagggcgataaaaacgggtggccaaaattggggcggcaatgcaagaagataagcctatccatcccttgcattggtattgtcatctggggcagggcaggggagggcaggagaagctatctattggccatctatccagcagccgctctccccagccccactctctccatctagagctccctaactgcaccggccgccacaccaccgtaccccgacccctcgacaacaatggccaccgtcctaggcagcccccgcgcgccggccttcttcttctcgccgtcctccctccgtgccgcgccggcgcccaccgccgtggcgctgcctgcggccaaggtgggcatcatgggccgtagcgccagcagcaggggcaggctgcgcgcgcaggccacctacaacgtgaagctgatcacgccggagggggaggtggagctgcaggtgcccgacgacgtgtacatcctggaccaggccgaggaggacggcatcgacctgccctactcctgccgcgcggggtcctgctcctcctgcgccggcaaggtcgtctccggctccgtggaccagtccgaccagagctacctcgacgacggccagatcgccgccggctgggtgctcacctgccacgcctaccccacctctgacgtcgtcatcgagacgcacaaggaggaggagctcaccggcgcataatcattcatgcccatccatctctatctctgtgggtcgtcgttgtgtgtaatttgagtacgcgcgtacacgtactgcttttgtttcatttgagcactgttcgtgtaagcgtcgagttgccctgccatcgtctctctctctctcgatctctactatttctgtgtgtgcgcgcttctatttttccaatgttgagttacgcatatttgtccagtaaccttgttttgcttaatcacacacagctcccaaattccagtcgttctgtggtgacggttggttgtggggtgtggcggcgacgtcagtcatgggagaagagatccgaatgcatgggggtgttggcgctttctctgtccctctccctctccctgtcttctcgcgcgctcgccgtcgtcatggctcgacgaacagacattcactgccttccccgcgcgcttcagacagaccacgcttgtacgtagcgccgcttctctagtcgccggagcaggcacgagtgggtggcgaagaggccacggcccgacctgctagcgccagcgaccgcatctcgccaccagccgctgcccgctggccgctgccaccgtatgttgggttgggctgaagcctgaagcccgtgacctttttctttttctttattctttccttttcttttgccggacaagacaagtgatgttgcaaaatccatataaaatcctagagaaactataaagtttttttttgttgaactccttataacaacgtctaccctttaatatatggtatataatttcacatgttttagtataaattgtttactgtatttctatgtctatatgctcttggtaagattgtttataaattttaaatttaaaatcttaaaagtcataaacttatagaaaaaaattaggatcctaaacaattttaaacaaaaaaaaaaacttttcaattacaaaatcgtatgtgttctttatgcatgcatgcaactgagaagctgcccaacggccagcgcagcagaagggcagagcggtgggcggccacgtcgccgccgcaaaggaactgacgtgtcccggcccgacgacgacgacgcgaaccttcaccgcgccgcgctccacctctcttaagctaaatacgctagtgcaggcgcaggcatccagctgcgacaacgtagcatacactagtgcttttgctagctacttgtgcgagaagagagagcgcacggcgatagatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 62 - DNA, 4146 bp, Artificial
  Vector
gagcgacctcggactcagcggctccgcctgtcggcgagcgcacgccgcgtgcctgcccgcggtctctcgtcgtctccagaagatgcgccccgatactcgtctttccacccttcttctacctccttccacccccttctccccacgccccatcggatcgcacgcgcaccgcaattgttgttcatcacctctccgaagaagcgtcacagagcaccgcccggaggttccccttcccgtcgccgtcgcttggccaggtaagtaatgactaatcgcctgagtctcctgcgcctcattacttcgaactgtggtttcgttagtcggacgtcggagcaatgctatgaaccttgccattccatgttacttggtgctgcgtgtccacattcgacgccacatttttttcttgctagccaaccattgcgcgtctgtgttacagttccaccggcgatatacattatgcgatcatttgatgtggatattggtgtttttttggttagcggatcaccttagttttgcactgaacatggaacaaatgtggatgttggtgtttttttgttagtggatcaccttagttttgcactgaagatggaacgaggaaatggggaaggggaactgaacgaagcacatagtttcagagactatgtgcactgcagagactatgcgaatggtcaagggaatttctcgccttggtttcctaatcctcgcttccttcttgtttcgaattcggtgtatatatgtggtttattcggcacgtttaggaatttagttggggaatggggcgcagtatgtcagggttgtgtcatctgaatatataatgcaagatctattactagtttaaattgacttattatgcaagtcgattaacctggctgggctcagtggtgtaccatggaagctttgtgattgtagccgggcagtagaacgctaaacctgaggtttggtactgtagcactcggctagtcagctgtatgccaagatgttacttactcgatactttctgccatgtctcatctgtgttaaccagggtgtgtgccacatagggcttctttccatgctgtttgttaacctgttcacacttcttccatatatatattcataatagctcgatctacaattttcctgtcttgagcttctttgttcctaaaaacgaattgtataccagcaatgtatatctgatgcaatattgtttgtaggcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 63 - DNA, 4510 bp, Artificial
  Vector
ctacatttatgttatagaggcgcaagtagaagaatgtgttataagttgtacatcggaaaaatagcatgtaaatctatagaatcaattttcatctttcaccccatgaatttgaggtatgcttatatgataactttagaaagtggtggaatgtcatattctaaaaaaaatagcctatttcattagtaagattccaattcctcgaaatgaaagaaaacaaacggggccttattagaatggaattcaattccaatgatccaaatggggcgtaagggatttccactttcctaagaaaaattagctcattttcccttggtgtttaataattatgttttgttaaaaggtcagcttcccgagaatgcgttgtgtaaaagcccagtataagggtctgtttggttgggctgtggctgtgaaaaaagttgctgtgggctgtgagctgtggaaaaagctgctgtagactgtaagctgttaaaaagctaaaaaccgtttggtggaaaccactaaaagtcgttaaaaaatcttcgatatatgttttcacagttccatccgaaaagccactaaaagcaggtccagaggtgctttcagatttgcactacgagaaagtcggtttttagaaaaagctgcttcctggatccagccctttggttggcttttggcttttaggggcacaaaagccaaagccaaaagtcaaaccaaacacaccctaagtcgcgtgagtgggccatttatctattccctatgcaaatctcctgaaaaattacaacattagctcgttaaaaagtaaaataattttaaaaaattagagactaattatttgataactttatagttcatcacattttcattgaaaatgttttattttttttgccgaactgatctacacaatatttgaatctacacttttggcttgggctcggagaaaaatagtgcgtgcccagccctttgctctttccccgataacaacgcggtatgtggcctaacgcccaaatccggcccatttattccggtgcgtagatgcagacgcgggagcaggtgccaagtcactggctcactgctgcaggtggagtggtggacactagtgccgcggttcatctgacacgtgtcgccacgtgccgccatggcagcacctcagcccggccggcgggccgactgacgtcttgggcaaagcggcgagcgacgcaggcggcgaaagccatccgatttgacccctcgctagacctttcaagaacgaacgctgtgctgctcagatcagaccgtgtctgcctcaaagcgatgccaggacgccacgtccaagcaaagcacccgatgccattgccacctcccagcactcacgcgtgagcgtgactataaaaaacgcaccctctgcatccgcccccgtctgcctgccctaccgaatctttcgccgtcccatcagcccagcaattcttcgctgttcgaggacccctcggtttcgaccgaagcccagcaagccgaccacacaccgctgccgttggttccgtcccaagagatgcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 64 - DNA, 3880 bp, Artificial
  Vector
catacgatttcctaagcggaatctcgagacgaaccacatttttcatccggtctatgagcgcgctcaatcgtcagtgacagagaacactgtaggtccttaactggaactaacaattcagaaacaatcctaaatgtttattacactggatggattttatttggatagatgatactaccgttcgtgggctaaaaagcaaggtaaaaactggcctaacgcacgtgatcctcccatggtctcagaagaaaaccactcgcaaaaaaaaaaacgcagcaatgaggctgctttcagttgacggtgtgcacgactcgctgaagccagcgtgattttgtcccggttcttggagcccagcaaaaagggcgcctgaaattccacaggttgaaggggaaaacggcaacaaaaggggctccgacatgcggtgcagaaagtgaaaacgcgagaaaagcgcccgtcctttctcgttcccttccgtgcagttgcaagtgttgccacgccgcgccgcgacacaccacgcaggaggcagcgagcgcaataatgcgcacgcagaggagcacagcggcgtcactgctgcggcagtaatggccgcctggttccacttccaccacggtgtgcactagcgctacaccgaggccaccttttcttggttccttcaacacgcctactccggcctccggatcgggcaccacgtctgccccctgctcccggaatctttgccaagcggcgccgctactgtccggcaaggcgaccccgtctctcggccggcgttgatctggtgatgcgcttttcacggtgtttttttttttttttgtttcactcccccctccgtcctgtgtgattgtgctgcagttttgtgccggtgctaacggaaaccggaaagtttgcgtctttatgttactgtggttgaggcgggcggagtactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 65 - DNA, 4135 bp, Artificial
  Vector
ccagccatcgtgcttgagtgaatgtttttttgcgtgtgtgtaggtgtgcgtgtgtgtttaattgatataaaataacccaaccaaacatctcctaagatttccataagacagagaggtgaggaatagtattagttctcaaatgaattcaatcaactaaatatcaccatcatatcaacattaataatagaacaataaaattttcatgatagagaggctggtaaactttattttttcatttgattccagtgtggaaaatagtaatataagcagtaaattttttcttaaaaaagagtggaatattcacctttttatataaattagttattcttcgaagcgtcacacacggatgtcctaaagatattcttcaactgcaacacaagcattctgaaaaggttagtaacaatcagattaatgttgtaaaaatatgtgacacgaaaatttaaagaacgcgaagtacaatacattggtaagaaatcaaagacacatattaaccaactattagagaacatatacatgtttcttctaatcctggttaggacataggacgagggtttcacttctatcacaatgtgctgcaactttcagagttaatcaaatctgttattcagtgttatgcagcgacaaattccaaatgtataatcccacgatgattcatgtgactccatttcgtctgtaattgatgcgaaactgccgaattgttactgctcccataagttaattgtaccacgcttgatgaagacgttcacgtatcttgtgttcgatttatgtagccgttattcagaccattgcagcgctgagcttcaaggcaattagttttgccccgcgatgtgaggcaggtgatgacacgcgcccttcgggccgatgccgacgcgccaccgtgcagactctaccgaggatgagagcagcgatcgagagacgtggagcgaggaggagactttaggagaggagagaccagagccttaagcggtgacacacccaatgatcaggaccacgcttgcacgtgtcaggtgcctctcctccacgcgtcgcgcgcatcacactgatactgtcatccagctcgacgctatataaagtggtaagcatcttcgtcctcggggccgccccaacggcccttgcgatcgccaccacgtcgcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 66 - DNA, 3530 bp, Artificial
  Vector
cattgttatacatcggtgatgcagatcttcgcttcgcatgggtggcaatttagtaacagaattgctctatgagttattgcttttagtttctgtctttccccaactgcgtttgagtttgcattgtcacatgtggccttttttggttgtaagacttgtttgatcggttgtggtgggctgtcatcctttggatgtaggccggaacctcttttcccattatctaaaaaaatacatcgtcatttccagtgagacccagcggtcgtgtcatcagaaaagcggacacgcgtcgcgtcgcgaggccaggtgggttatgcacggacctgctatgctagccatgctccaggctccagctccacccgtccaccgacttttcttctcaaccaggctgcctcctcaccatgcacgacgacacgtgcgatgccatcacgcatccacactcacctgttgcctctatatccaccgccccaccgccgtagcaccagaaagaaatgtacagtctacacagctgaccagagagctagtgcaagcgatctagcaagttttaatcttggaagaagccagctaggactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 67 - DNA, 3995 bp, Artificial
  Vector
atcatcaccctaccccgagctgactcgggccgcagggaaccagaccggtgtcccatctggctagctccgccagataggcaatgaaggcgccccgcatgctctgtgacgacggcggctctcagcccccttacggaagcaagaggacgtcagcaaggacccaaccgctccgacagctgtccctccgccaggctccatcgctcctccgacggccacgacatcgcaccagctgggtgccaaaatctctccggctgccacgacggcatgtacttagggcgctagctctcccccgctagacacgtagcactctgctacaccccccattgtacacctggatcctctccttacacctataaaaggaaggaccagggccctctcagagaaggttggccgcgcggggacgaggacgagacaggcgctcgcttggggccgctcgctccctctcccacgtggacgcttgtaaccccctactgcaagcgcacccgacctgggcgcgggacgaacacgaaggccgcgggattcccacctctctcacgccggtctccggccgcctcgctctccccccttcgcgctcgccctcgcgctcgatccatctgggctggggcacgcggcgacactcactcgtcggcccaagggacccccggtctcgaaacgccgacaatatgatatatagaaactataatgatacacatgcaattaactagtgctgataatatatttacactgtagatttttccgataagtagaaaccaaatatttcaccgccacaaaaggcttcatcccatcagttacccatgtagcaaaaccaaaatacttgtgtgcgagaggccaagagcccaaaccaccgccaaggaaacccccagcccaccaggcgccttcctctataaataccctaactccggtggccgaagttcctcacccattcactcactcatctcaaggcctaggtagcaccgtagcagctactagaagcagctagccagaacaactcgtcgcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgacgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 68 - DNA, 5523 bp, Artificial
  Vector
cttccgataaaaatatttggaaccgcatcctgcatatttatttagaaacattgcatcgataaccgttgcagagcaatatacagggtactcaccaactaaatccataccatacagtgacaggtgaaattggaatacttgatcagcttgaataacagagagcaaagataaatctagccctggtaataaccatggctcacatcttatgtacaagagccaattctaaagacacagctgcgggatgtagcagttaggcaaggaaagaaacaaaattaccagctaactgctaagtgtcaaaacattgctttacactaaagggttaaatatttgccaagaaggtagtgggcacctctcctctgcaagcagcagcataatctagggcgaggcttggtgcagcttccttgtaggggcatatgaacttatccatgaaggcactctcactcatctggacagcagtatagtagctccggtcctgctccaacaatccattttccttcacgcctagtttggatcactgtaattgaattccattctaataatagtaatttagacatatatcaattaagataattcacttttgtacaaaatatatttgtatactattattagcaatatatcctaaatatttatgtgctacatttttactatagaggagtagaacgaagagtgtcatataagttatagattagaaacaaattctaatcatgcataaaatcatttcccatcctccaccctatgaatttgagataggcttatatctgaactttagaaagtggtggaatgtcaaattccaaactaaataagttactttattgagtgaattctaattcttttgatttgaaggaatccaaacgccccgtcaggtaattcacaacatagtgtcggggcatgaggcggcgttccaagctatatgtaagcagagcaggcctgtgagcaatgtactctggctccaacccaaccttggtgatcaggaacgcggatccgggcgtcgccgggggcgcgaccctgtgcttgccaaattgaaaatttagcaaagtattttttttaccttgttgattatgttggataaccgcatgtccctaatttgaatgatattgctccttatctctaaacgaccaactgtagcttgttctattgaaaactcatcagaataatccatagagataacatcaccaacaaggactgaatcatccagagatgcatcaatggccattggagctagggaagtcggcctaactaccttcaacccgacttggccaacttgtacagtagaagacaccatggaaatcgatttaacggactctaagggagtgtttgaatacactaaaactaatagttttagtggctaaaattagttggagacatccaaacaccctagctaatattttagctattaactatttttagtaaattagttacaagttagctagctatttattagctagctaattctactaacaaatttttagctaactaacgcattcaaacactgtctaaacaggtaatctaggcacgcgaagtctgactggccagcccaaactcagcctttgtagtagagagagccgaccttggcggcctgcacgacatttttgaactttttttatcggtttggtcttcattactttatattctttgtcaaccaaatactcccttcctcttaaattataattcgtttgattttttttatgtaaagttttatgtatagtccacctagttaactttaaaagttggttagtctagattttatggaggccgactacaatagcaaatctaacaataaaaaagttagctagatcagatttcaagaacagtctgaccgattttgaagatttgattcaggttaattctacgattttgaagatccgacctaacctaattttacgttcagactaaagacatgtttgtttaagattatgatatgtctagttatataatttaacttattttaaactaacacttaatttaaaataagttagattatatgatcagaatattatatttatataattccaaacaaataactctaatatcgattctggtttatattgtataatttttatgttgaatattacggcgcgccaacaaattgcgatcgttatgtaatcatgtatacgaccgactgtactattcataaaatataattcaaaataggagatgctcgagatagcctggcaagggaggaggtatcggaggcggcgtcgcctgccgcgagcgcaccatgcgtcaacgctcaggacctctcgcaggcgcgctgttggatcgtgagcaagcacgcgtggcaaatggcatgcattcctctcgtgcaagtgccgagctgagggtcccgacgcggggggcaggacgtggcagagcgccgcccaggcggacgccaagtgccgagctaccagggcaccttctttattccgtccttgctcagtcacacctcgctctcgctcactctcgccgtccgcacagccgctcatcgtctcccactgcctgccctctccctgcgccggccggtgccccgacgcgccatgtcgtacgcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 69 - DNA, 4305 bp, Artificial
  Vector
tcatcggtactcgcgatgtcgtgcgcgtccgacgactactatatcaaggactatgacgtcatcttggcaccagaggtgacacagaggaggaccaacaacaataaccactcgccaccgtcggtcggtctgcctttaattctcttcgcaaacacatacacttgcttttttgtttaagcaagcgtgtatgatggtgcgtacctgatgactgacaatgtacgagggaacttctaccggcaggtgtaacacgtgctcttcaatgataagatcatgcaaatcgtgtcatatatcgaagcctgcatgatactgatggttgaaatatagtagtgaaacagctaaattaaaataacaaaatttatgtatggctaggattacaaataaattataaaatatttttttataacaatataagacacattttgtatataagttattatattattatatgtttctgttgcaacgcacgagtactcacctagtatatccataggcttttgatcctctacgtcgagtttttgcttgcatgagagtcgacctcttctcttttatcccttcttctctatcttagcatttgctcgatcaactgtaaactaattattatacgtacttcaagtcatattgaaaccaatggcatttaaaaaagtaaccatctgaatggaaatatcaagctaaataaagaagttacgggagtatgtgctgagaaaacctagacacattcagagaagttaccaagcaagccttaaataacaatttttggtgctttattgcagatagatactcctataggttggagagaagggcggcgatgctaatgcaccatgcatgtgtgtctgcccatatactctatcttatctgaatcctaccaaactctttaatttgactctttatgaatggaattcccatatgaattctataacacaacagtacgacgtctactggagcatactgccaagcacgctagccgacggctcggcgaccgtctgaggctgtccatgcatgcaaatgtgcacacatgtacgccccttttagcgctggcatgcacccacgaccacgacacggctcccgtggccgtgaatctgacatcccgggactcccgcctccggccgcgcgtgccgtgtcagcctccatcgagcggtgtactccatgcaagcctcgacccgggccacgtacccccctcccctccgctacgtgtcaccgctctcgtcacctatatatggcggtcttgtccagtctttccacttaactactccgtcactttgtttgcaaagctcctcctcgatccatcgatcactgcaccggccggcggcaccgcgcgcgctcgcaggggctagccaacgagacggcagcaacgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 70 - DNA, 3726 bp, Artificial
  Vector
ctcacacaaatctaaatagtaaagtagatctggttacagagcagaaattttgaagtgtcatagataggctgagaatatgtagtcacgagcaacacattactacttgaggccatcaatactgttttttcttctccaacctatgtaactatgttattcttttcctccttaaatgtaaggcaaacctcctatcctttttctttcaaattcattgcattaactgagtggttcttacatgtagaaatgaagttggagcaaagaggcttggtgcggaaccgaaaatttgttagtctataaatcattagagggcgcctgaagttcgccaaatgttctagtagattttagtaaccacccacgctagctgactcgaccaccgtctgctggctggcgctgtccatgcacatgtacagcccctggcgctggcacgcacccacggcatacctcccatgactctgacagcccgggactccctccgctcgtgccgtgtcagcctcactcgagcggtgtactccatgcaagcctccacctcggccacgtaccccctccccttccttacgtgtcaccgctctcgctccctatatacggctgcctggccaggcctttccacttcactactctctctctctctgcctgcaaagctgcttgatccatcatcgatcacatcactccaccagccgcagcgcacgaacgtacgactcatggcagatcgcgaccgcagcggcatctacggcggcggcgcctacgggcagcagcaggggggcggccgcccgcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 71 - DNA, 4959 bp, Artificial
  Vector
ggttcaagatatgtatgtgatgaacataattttactcaaaagtgatggtttcattgtactaactagcatcaatacttatgccagagtggagcagtgtgtctatgcactactccgtacaagagacatgtccctcacgctacaaggagtatgggatcccagttgactggttgtctgattccggagtagttggcaaggtgtgcatctctagcatacactccaatttcgccaaacatgtgttaaaaacatgcatttttaattccttatgaaggtgggtatgattttactcagagtatgcacctaaatgaactcaataatttatttgcatgttggatatatcaaattggcatctgttcagcttgcgaacaagtatatgaagagggttgcatcagaacttgatgcgttggaaggcactgagaaagagcccaatagagagtttttgcttctccagggtggcagggtgtgaggttctctttccgtgttcatcaggtaaaatcctcacgccaatattttcagaacccaggtacttcacaggtgatatgaatatttcgaatcagtaacttattcatctaccttctgcagtttgctgggggatttgatgcagaaagcatgaaagcttttgaagagctaagaagcaagatgagtacacataaatctgctccacagatatctgaaggctgaagcatgagttgaacacaggattcaacacacatctggtcttacttgctggatactgatttttagttgtgtacaaaagagaagtttaaattccagtgtggtgtcaagcaagttgtatatgctaagagaatacaagtcatcaggttatgttcatttattttttatgaaagtaagggcaggacaggttatgttcatgcatataacagttttagctcaagtgtataccttttcattttaaaggctgtaactgtaaacaggcagctccagaaaatgaatcacttgggcatgtttttgttgctacgcatatactactaataaaatgtcaaaagcgtccattcggttagtccggtacccatggagataattcgtctgaatgaaccttgccgtctttgttaaaagaaaaaaaaattactttgtgcctcaccgcattcgatactttgaagaaattgacccgttaccagttaccacccacgcacatacagtgctagaacagcagatctgacttgaagaagcatatatcaagacaaaagtccacctgaacggagacagtcgaggttgtggcggagagctccacctgaacgccgaagtggaaccgaacagttccagtggttggccgtggcgagctggccacctactagtctccggggagggggctggctccagcgcctccgagcgcggtgccagtgtcgccgtcgcgtgggttccggttccggccgccgccgcttgccggggaatccaaacggcagacggggtcgccgtgaccgtgaatcgtgtttacttgggccttatctaggccacaacgtttgtgactctgtgagcttattgtttgggcccatctctttggcgtgtctgagaacgaattttatcatcaagcccacgaagccaaaccaggatcctacccacctagcacatactagtgaagaaggtaaacaaagtgtattttcatgttaattcacatgcgcgtggtcgacaccacaccacacgacttaaaactcggtgttatgttctacgaccacaagagccatgttaattttaagccgtgtctgcgccatgatccgaagagcgcgtacagtgagagaagaaacgcacggccgccagcagctgccgcgggagcgaggcagcacgtccgttggtcggcccgggcgggagcgaggcacacgtcgcgcatgcacgcgcggcgcccggacacgtgccggcgcgggcacgtccctctccgcgccttatactcctccccgccatttcccagcagccacaacaagacagagagatacccgatcgccaccgccgaccggactcccctcaaaggcggccacagcacagcgagccgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggacctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 72 - DNA, 5084 bp, Artificial
  Vector
actaggttggtgagatccttaggttttggaaggctttatgtggcagctctgaagactccaacatctatggcctctagctatgcctccatatgtattttgtaataagaaaaatcatctcccttaaacatgggagagggttcatctccatcgaacatctttctttaggtggtgaagcctaaaataatgagcacgacactttgatactaattgaaaggattactatgcccaagaggtagggtggattagaccaatctaaaatttcttgtagcaataaaatcttatcgaatatcccacttcaccacttgtgcttaagagtgatatatattctatcacaaaaggttttacaccctagtttgaaccctattctagtatgttagttctagtaatgtaaagacaagaattaaattgctaaaatgcaaatgcttaagataaagaagggttagaaacatgacagcatttttccaaagtattaaagagccgggactctccaatagtcctcgttggagcacccacacaaggatattgctccccattgaacttcacaaggatcaagggctctctatgggtttgttcctcgccactctagcatggtgagtcacccacagttgtttacaccttgagtggtgatatacactagatttcttaagtgtgtatcatcagacttatttaggtcaaactatcattcatggcccctttatagtatagtcaaaggaaaaaacaaaggtgctatcactagtatatatgtctatcaactctattaactattagaactagccagtccttaacctttttgctcatccttctaaaatcaataattgaataccatcaataggggcacaaaaactatatagccaattattattactcatagccaataatttagtgttgtcattagtcactaaaactagatgctttcatatctatcatgctcgtgtttgcctgatccaattcctaggtcatgtttgggctccacttagagcttgtaatggcatcacaacatgatctgattaagaaaatgttgactaaagaccttctcacccacatataaaatgccatgacttcgctcaaacttcatcccctagtctcctactacacccatgtctatgcttcgcaacttcacaagtctaccacatgtacttgcaaagggctgattctcctagctcccatgtcgtcgccatctttcccctttaccaccacaaaatcacaaatgtgctcacttattatccccgcgtcttcccttccctaccctaaacactatgggcaacaacgtcgcatcacttggtggtccttgtagatctagacgaggtctcctccccgttttgtgcttgttgatatagtgatcttgtccttgtcttgtcgctgtcctttggagtcgtcgcagagaggcggtgaaaattctagccgcttctagaaggtgcggtcgccggtcggatgagagtcgagtgagtgcaggcctataaaaagtagcaattggataacattttgaaaaaaatgaaagaaataaaaaaactagggttaaataagattctaaattaaatctaagaaatctatagacattcggaaatctatttatcttttgtactcctttgattggatttctagattttgacgtttgtgtaattgggcttgacagccccatcaatacaggcaggccagctgatttcgggtgccgctgatttggcccatggaggatagagcaggcctttcaacaggcaggcagtccatgacgtagtgcctcggcctcaaagcccatgcgattaatatccactggtacgtacgcgacagccgagggagtcactgagtcagtgccgaacggcctggaaaccccccgccgtgtgccggtgtccacgcgcgccccggtgcggccgccacgaccgcggacacgtgccgggcctcgcacacgcgtcccccacggccccctcgcatcattaaaaccgcggtgcccgcgccacggatccccggcacttcattccggtttccggatagcttacctcccatcgacggtcggtgagccgttggtgagtggcgagacagcgggtgcgctgggcaagaacaggcgacgaagaagatggcgccctaggcggccgcactaagcgctatttaaatgccagctgtacactagttatcgtacggcctaggccttcacctgcggagggtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcagaagggcgaattcgacctaggccaagtttgtacaaaaaagcaggcttgataaccaaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaaacccagctttcttgtacaaagtgggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 73 - DNA, 3185 bp, Zea mays
ttggttttttgataatttgtttatctcacatgatggttctaacaatatgcgcagatcttatacatctctctcgtagtttttcataaactacgagaaagatataggttttatgaacaaatttacttttattttgtcatataatgaaatggtcaaaatataaattgtacatcatgatgagttatacaaatttgtagtccaaaatcttttcatttaaattaatttactgctttaaaatgtgatttttaattgtctttgcctagtgttgaaaaaaagcactcggcaaagaaactctttgccgagtgttaaaaaaacactcggcaaagaatctctttgccgagtgtcaaaaataaaatatttggcaaatagcttctttgctgagtgtttttttgcttagcacttggtaaagaacttctttgccgagtgtccgaaaaagcactcgataaaatatttggcactcagcaaagaaccgaattccagtagcgtgagtcagaagttggcatttgaaccctttgaaataatcaaacaaatcaacgcagttgcatatgaattggctttgccacatggtagtgcagtccatcgagtgttccatgtttcacagcttaaactaacccatggttttggtaacagaacacacaccagtgactccatcgttaccggattttgatactacactgaaggttccggaacaaatactggattctagattgattctagattgcttcgatcaaatggggatcatcaacctaacacagcttaaattatacatgcatgcgtacatttggtccttcaagaggaggcaggtgatgatcaaggtctctctagcggcctggtgtgcagccacgtacccatccacgcgtcgccgcgggcgacgacaggtgttggccaacctggacatcagacaagcacgacaatgacaggcgttcgccaagcgtacgtacaggagacgaacacgacaagtagcaccgtcctggccgagagcccgtcatgctcatgcatgcatgcatgctgatcatgcatactcgatwaatatacagcagagagctgagctacgtacacaaacaagaagcttgcattgttcgttgctcgatcgttgcaggtaatggagaaccctgctccgagtatcgtagtgccccccatcgccgcacctgtatcagcgaacttcagcagactcgccttccgcaacctgtacatccgacggacgggtccagacagcagggagatggtgactgtggagggaagaagaggctctagtgatcagacaggcgacatgaggtacgtgagtgacttccccgtctacgatggccgtggctccgacgccgtcctggtggctcgcgtgcagggcgtcacaaccacgttcgggaactccaaccagttcttcaccgtcgccttcgaggctggcaggttcttcttcgtttagacagatagctagatagatgctagcttgtggttcatatatagcagatagatatatgcatgcatctgccattgatcgtccatcgtcatcgatcagcaggctaaagggctccacgctccttaccgaaggagtggtgacggatggatccgatgaatgggcaatctacggcgggaccggagagtttgctatggcgagaggtgtcgtcaaaagaaggtaccttgccgatagggacggcgccgggaacactgacgagctcagcatgcaggttttctgcccggtgtttggctcatcacagcaaccaaacaagcaggcacgtgctgtactgtatatatgtccgagagagagaaacagacagtagaatcctcatcagtagtataatcagctgcatgtatgtaggactccaccatctctgtcaccaagattgggttatggggtggcgaaggagggtcggcgcaggacatcacgacgacggagccaccgcagcgcctgcacagcctcaccgttcgtgccagtgccgccgtcgactccatcgagttcacgtatactgacagaggtggccagaggcgcgccgctgggcgatggggtggacttggcggcaaccttcgaacggtgagcagcaagctctagcccctacctacctacattgattcatgaagcatgtatatagcttcctcatgcatgcatgcacatgcatatgcgcgcagatcgatcttggcgacgccgaggatgtcagggaggtctcaggaacgtacggcacatttgaaggagccaccacgctgacctcgttcagaattctcaccagcagcaggacatgggggccatggggcgtcgagaacgggacgcgtttctgcatcaccgcgccggtcggcagcagcatcgtggggttctatggacgcgcgaccagcaggctcgtcgctgcgcttggtgtttacctgcgccgactttgatttgatctgaacagagcaggctacctactagcaattgcctgctcgatcaattatctttcgcaccctttattattaatataatctcgatcggtcatcgtactcctatgtcaatgtcaaataaaggtcatatgtagtcatcgatattgttggatttattggcatggtctaattaagaaattcatttaattcctgaaaaatcataaaagctgaatccgacgatcagatcgcaacctaatcggatcgtgagcttatcgcttcttgatcgtgagcttatcgcttcgtgaacgtggatctatataaagtgtctaccagcctttgtcacataccatcttgtcacataccatctcatttcaaccgagttaggttttgtctctgcttactgttgcctcgttgccagaggcggacataggtagagcgggtatagtttcctcgctcatttatatattgtaaatagtagagcttaaattttcatcataagatatcctgctcagtttaacttagatgtctatgctatgctatggtactttggtgttcgccctacaaatatgccttcaatcccattttgtcttaggtccacccttagccgtcgttgtggccttcttcatcccgagctccgacgtacatctacgacagggagagcatgtctctagaacctgacaccttggatatcctgcaccgggagagggcaaataaggtttctgggaagtgtcttcacacgactgctcgcgttcttctcatcaccttgtcgccgacgtgagggcaaataaggtttctgagaagtgtcttcacacgactgctcgcgttcttctcatcacctgtcgccg

SEQ ID NO: 74 - DNA, 3546 bp, Zea mays
  misc_feature (3048)..(3048): n is a, c, g, or t
ttagtagagaataacacacatctccctaagtatcacaaaaaaattaattttaagaggcaattttagtttccaaagcaatttattaagatttaagggagggtatttattagaaggaagtacattgatcaaacagagaaaatttcatcattatggtcccccatagaaatttgtcactcgattgtcgcaaaaaaagttaccagtaaatttgataaattttggataccggtctgttttaactcatcaagaatagcacgacgaaataatagattgcgtacgttttcattcataacatctctcactaaagcattttttacacacgctattaaaatggttcggaaactgatccttaagcggtctattcatatccatctatcgttctaagattcttcctgtacgtagattctttccgattaaattctcatgaaaaattaaaaacaaacaggtccttaaactagtcgccggaagttctcgcttttgctgtagttgtagtgtcgatgcatgggtggaaacgtggacagatagattcatgaaatgtggacaacccacctggtccagagcatcatgatacattgatacacgtggcacgctgcccactgatgccctcagctttgaagctaacgtgtcaagtgtggccggcccctgcattgcgtcgtgccaatcgtccaagtcccatggcaaaaacccagcgctttgtgccgccgccgtccgccggcccctctgcccttgtacgtgcacctagacacatcgtcatcgatcatcacacgcaatcgacacaagaagttaataaacagcccaaggacgcagagatcagctgatcgagaaggacttgtactactactcagtattgtcgtcacatgcacatatatgtacataaagagctagctacctgagctctacccaaggtcgcgttgatcgatcgatcatggcgcggttcgtggacccgctggtggtggggcgggtgatcggcgaggtggtggacctgttcgtgccctccgtctccatgaccgtcgcctatggccccaaagacatcagcaacggctgcctcctcaagccgtccgccaccgccgcgccgccgctcgtccgcatctccggccgccgcgacgacctctacacgctggtatatatatgctggcacttgtctgctcgatctgtgcctccagttgacatgcatggatggattgcacaacttgctgcgtgtcgcagatcatgacggacccagatgcgcctagccccagcgacccgaccatgagggagtacctccactggtatgaactgctacagcatgctacgtacttgtgtcatgatctggcagcgtgtgtcgccaaagagtagatgcactgtatatttgtacgtactactcattaaattatactgtatgtatgtatgtgtccaggccagttgcctaatttctattctcttgcattgttgccacacatgcatggatgtttaatttctcggtcccagcgtgaatttgtggcgtgcaggatagtgactaacataccaggaggaacggatgcaaacaaaggtgagtttaatttaaagataattacttaattagttatctacttatgtactctctctctctctattttaaaatttaacatattttgatttttttagatacattgttttcctaaatacatagcttgtacacataagtgcatgtgtgtctcgaaagaaacaaaatatgttataatttttaataaaggaaatatcaaataatcaatttaacaaaatatgttataccacaactgcatggcacaaacttccatgtgagccgtgatcgcgccgttcccgagttggcgccgccagcacgtactacgcgctgccactgcacggcacatgtcgccgcacgccaagcgtcgtcggcacagaagcgttgacacctgttgggggctactactggtgtgtgctgcaggtgaggaggtggtggagtacatgggcccgcggccgccggtcggaatccaccgctacgtgctggtgctgttcgagcagaagacgcgtgtgcacgcggagggtcccggtgagcgcgccaacttcaacacacgcgcgttcgcggcggcgcacgagctcggcctccccaccgccgtcgtgtacttcaacgcgcagaaagagccggccaaccaccgccgccgctagctagtagctccaacaagggcgcgccagctgagctgcgtgcgtgcaacccaccacacagccgccggcgaaggctgcctatatgaccggcgaataaaaagtcttactgcaccgtccgtaagcgtactctctgttggtatatgcttgtcttcaggctcttgagtctatctacttaaatgtggttaccactgagtaatagaagcagttggcgcttcgatcgatcattctaatatccgtacgtgtcaatcattcctgtttccatcatcttgcatttgaagacgcattggttctacaccaaggtgtggtaggcccactttggaattttcgggaaaaaaagaagaagtttctgttcgtacttttttccctgtgagaaaatgaagcgattcactgaaattcctgtataattttctataaaatttgcgttccaaagggcccttacagttggatcagtggtgggctttctgacttttgaagtttagtttggttgtttactgagcagtttatccataccattcccatgttcgaactccattttacgaacaatacagtttatagtgcaaaacagtggcacaaaaatagagtaaactgctttagtctctattccctcgagaggtgactaaaaggaactaaatcatattaattctctcttttgcctcttatttatttcagttgcattaatgataggagaatgataattaatacattctgaatacttttagtcactcataccaaacgagtagggactaaactttagtctctaaactaaactttagttctggactaagaaaccaaacagtccctaacagcagttggcgcttcgaccatttttcagaggtgtcaaatcatgcctatcttgataatttcacaaataaggatatgttcattgcatggtgttgaaggtctaaagaaatncgtaacgtatagaaggagacctaattttatgtagaaacacataaaaatgccacaatataaagtgaagtgatcaatttcaacattagggcatcattacttcatacaaccctagaataaaatagtatagactcatatgatccaattctctcatatgcacatcgatgtgtatgtaaaattaaccttgattgacgacatatgtatgtaaaattaatcacacgactgttctactcccttccatatggtctaagcaatgctttatgagaccatctgtgtgcatgaaaatgatggatttattatctgcatgatttttagatgctatgtgtcggccatcaaatagaggtccatgtgagtgaattgaagcctagatcaatccatttttacatttttattcccttcgccccaggctcttgtttcccatatgcgcacagcatcaacctcattcacctctatctcctatcaaatcctttctttctctaaagatcctcctaaagtcgctac

SEQ ID NO: 75 - DNA, 4160 bp, Zea mays
catttttttgttatatacagcttatccaacattctactatattaagacatttttttggtgaacttcttgcgaaatcaaaaggttctgctcaaatatcatttctctccattagctgctctgctgccttgttttatcttatgacttatgttccctattctaaaaaatatgctctctgcactcacctttatattactgcaagtttatttatgcttgactgaatattcatgtttccatggtatgcgttgaattacggttttgtaacaggcgtactgtgaagaacatcccaaagtatgcttcagcacagttttatattgataatgtacttccaaggatcaaagagaaaaggataatgtctatcaagccatttgtggacagattggggtaagtaaacttttgacgacaacatttcggtttttcctgttcattttaattttgattcagaacatctggtcagcttaaatgatgtggtaaagggagcattattcttttattttgttctttgttctgtgcaatggccatatattttcgtattgatatgctctcagtggaacagtctgaagctaatgtgaaacttcgatgttcaggtatgataatgtaccaacagagattaaccggctgagatgcagagttaactatcatgcattaaagttcctacctgatattgaggaaatggctgttaagcttgctgcaagaatgaggaacaaaactggcagcataaatccatacatgtacgttataattatccttttcgctgggtgatattgtgtacagtagagcttgtatctacccaagacactatccagtgcatagctgaagttgacgctctcaaagtttatttcacaatcaaatattgacacattttttttgttcttaaagggctcttcacctgagatttgaaaaagggatggtggggctttctttctgtgattttgccggcacacgggaggagaaggcaatgatggcggcttataggcagaaagaatggccaaggcgcttcaaggtgacgcggactggactagatacatctgttacattggactgagaaagaatccctaggagaaatgaagcaccgacactaactatacgcgctagttcgggaaccccgttttctcacgagatttttatttttccaagaaaaattagttcattttttttaggaaaatataaatctcttaagaaaatgtagttccgaaactagccctacaagtctaacgattttctcttttttgtatctgccacgcagaatggatcccatctatggccactggcgctgcagaagagaaaagaagggcggtgccctcttgagcccggtgagatcgctgtgatcctgcgggcactggggtacacgagcggcacacagatatacgtcgcgtccgggcaagtgtacggcggcaagaaccggatggctcccctcaggaacatgttccccaacctggtaggcaggctcaaatccacttcagattgtgtcaagctgaagccagtgataactgataagccactgccgtgcattgtgcatcgctgcaggtgacgaaggaggagctagcgagcgcggaggagctggcgccgttccggcggcacgtgacgagcctggcggcgctggacttcctggtgtgcctgcggtcggacgcgttcgtgatgacgcacggcggcaacttcgccaaactcatcatcggggcgcgccgctacgcggggcaccgcctcaagtcggtgaagcccgacaagggcctcatgtccaagtccctgggcgaccccgacatgggctgggcctccttcacggaggacgtcgtcgtcacgcaccgcacgaggacgggcctccccgagcccaccttccccggctacgatctctgggagaacccgctgacaccctgcatgtgcagggcatgagacgggccatccctcagatcagagtggccgtctggccgagtcctcgtcttcgctaggctacgactacgagtagtctcgtgctcacttccatcagcgcgcatttcagcgagcatatgcttctactagttaaacacctagatttgtatgtatttggctggcctaatctgtagtttcacatagatcacttcatgctgaaaatgtcttggacaaggcagtgaatgaagataacatcagtaacgacctgcttttccaaagctcctcttggatttaccaagctgagccaagaaatggctaacctgagttttgacttgcagccttgcaggcatgacgtgggcgacctgaactcgctagtcgtagagtttctttccacgcggcagctcggcagagctacgcagcctctctgcccgccacgttctcctgcaaggctgcaactcgaagcactgagaagctattaattaatggattctcgagcatgcggccttgttcgctaaacctcgcgcacagaaacacaagcgagtaccagagcagctaggagtggtcagcaatggcgcagcttaaagcagccgccgacgagcagcaggaggcggcgctgcacggcgtcgcctccggcggcgggaacgagagcaacaagaaggcgcgcgcggggctctgcggcctgctccgcgagcgcaaggtggtggacctggcgcgggccaagcggcggctggtggaggttccctacaccgcgacgctggcgcacacggccaacgcgctcctggcagcgcgcgtctctgccgtggccgtggctgcgccaccgggccactggatcggcgccggcggctccatgatcctcgagtccgacccggccaccggcgccgtccgcaagcactacatcggcatggtgaacatgctcgacatccttgcccacatcgccgaggccagcgacgacgacgaggcggccgacctcgaccgccggatggccgtgccggtgtcctccgtcatcggacactccctcgagggcctcactctgtggacgctccacccgagcaccaggtacgccggccgatctccagctcacactcgtatttttcttcttcttcctcgcattgtacgcaatctgcgcgtgtgcgacatgttcgttgcgttgcgccagcgtgctggactgcatggagacgttcagcaagggtgtgcaccgcgcgctggtgccgctggagagctcggcggacaacgtggtggcgctggagctggtggagtcggcgccggggtaccggatgctcacccagatggacgtggtgaggttcctcagggcgcacggcgcggagctcaggggcgtgctgttgcgcaccgtgcgcgagctcggcgccgtgaacgacaccgtgttcgccgtctctggcgttaccaaggtgatcgacgtcatcaaggcgatgcgggcggcgtcgctgaccgccgtgcccgtcgtggacgccgtcggtagcggtacagagacccttcaatacgtgagcactgcacgcgcctccgatccgaacacgccatctcctgccggatttcctcctgtctgaaattcctgtcgtgctgctgcaggggagcgggcaaagggcggtcgagacgttctcggcgacggacctgcgggactgcccggtggcgcggctgcaggcttggctggggatcagcgtgatggagttcaagaggaaggtggctgagtaccgcgccagcaccaggcttgtggtccccggcgccgacgccacggacactggcacccctgtcgccgggtacgccgacacccccgcggctgccgccgccactgacgaagagcagagcgagcagcaggagtcggcgctggtgacgtgctcccaggagagcaccctcggcgaggcgatcgaggcggcgacatcgaggcacgtgcaccggctgtgggtggtcgacgaggaagggctcttgcgtggggtcgtgtcgctcaccgacatcctccgggcagtgcgggaggccgcgctcggcgaggacctggagctgcacagcatcgtgccgtgagcccgtgggccgtgagtcgtgacgtccaagaacaagctcgtagctgtttctgcccatagtgtccgtccacctgtcgtgctcctgcagaggagggggaaaagggcgatcgagacgtgttggcgacggccgaccggcgaccgccgccgagctcgttttgccttgttgctttgtttgttatatggccctggctccgatccgggcttcgtatttcgttgacaggccttcatcgtgacgccttattttgcatcgttttattgatcaaccaaaaa

SEQ ID NO: 76 - DNA, 2131 bp, Zea mays
cccgtcatgataataatattttcacatgcggtttcttaagcaaaccgccagtgctaatgatatttacactagcgggctgctaaagaaaaccgccagtgctaaagatatttacactagcggttggtgaacaactgcctgtgaaaaaagccgatttctactagcccctagctagcactggcgacataaaaaacgtcagtgaaaatagctctaggatcgtcactatagagcttctatgtacttagtggttagaactgatattgtagtgcaccaagtgccgattttaattaaaccaatactaaatactagtaaataatactagtggtctgaattcgatttctatagtaatgtttgcttgcaagccgcaaatagagtaaacattcgtcgtcacagaaatccacattacatcaaggtccatggcggccggccacgtacccatcccacgcgtcgctgcggaggacacgtgttggctgaccggacagttggccgatcagacagtggacagaccggacaatagaagaagaagacgacgacggcggcggcaccgccgagtaggtgcatggtcacgctagctgtagctttttgcagagcgtcgtctgtaaatacgtagcccttccacaagcgaggcaaggggggagagagtatcgtcagctagcagagagagtgcgtagcaactagcaatggcggagaaccctgctccgagttccccgctagtgacttctcccgacgccgctgcgccgcctctgccagcggagttcaccaagctcaccttccgtagcctgtacatccgtcggacaggtccaggcagccgggagatgactgtggacgggaggccgtctgaccagctgggacgacggttcgtgagcgactttcccatatacgatggccgtggctccggcgcccacctcgtcgctcgcttgcagggagtcactgtccaaatcggcagctcgcaccagctagtcagcatcgtcttcgaggccgagaggtagttcaatgtatgcatgcatgcatgtagtgtgtatatatataatgcatgtgcgattcattcattggttcatcgtcgtcaggcttaagggctccacgctgctcaccaacggagtgataacggatgggtcagacgagtgggccatctacggtggcaccggggtgttcgccatggccaccggtgtcatacgaagaaggtttctcgccggtagcaacgacgggaactctgatgagcttaccagcatcgaagttttctgcccggcgtttggctcggcacaacaacagccaaacaagcaggtacgtagtgtgtatatgtcagagaaaaaatcaataactcttttaagaatgacagcagaattctcagtaacacaatcagctggatgcacgcacgtaggactccgtctctgtcaccaagattggggtatggggtggaggtggagggtcggcgcaggacatcacgacgacggagccaccacagcgtctgcacagcctctccgttcgaactggttttgccgtcgactccatcgagttcacgtatactgatagaggtggccagaggcgcactgctgggcgatggggtggacttggcggcaaccttcgaacggtgagcagagcagcaagtccctacgaccctacctacctttgattcatgcagcatgtatatcttcctcatgcacatgcgtgcagatcgatcttggcgacgccgaggttgtcagggaggtctcaggaacatacggcatgtttgaaggcgccaccacgctgacctcgatcagaattctcaccagcagcagaacctgggggccatgggggatcgaggacgggacacgtttctgcatcaccgcgccgatcggcagcagcatcgtggggttctatggacgctcgaccagcaggctcgtcgctgcgatcggtgtttacctgcgtcaacaactctgatctggtcatctagctatcatcgtcgtccggtgctgtcgtatgtgtgttcatgtcaaataaataaaagtggtcttgtgtctacggctgccatcgccatgtgccaaataaatgccatgcatgcatgtgtctaaatttatatttccgtcgtcaccctgtagtatacgtatttatatcaaatataaaagcctgctgcaattcactaaggaatcgcgttttt

SEQ ID NO: 77 - DNA, 1996 bp, Zea mays
gtgattaagttgactggcaaattgcatagacgataatacactctatgcttttgagataaccactgtacaccaaccgacttgttgtttgaaggctgatctttaaccattactactgtaactaactgttgttgaataaactattcatacgacttggagactttgatgtatattatgttttgtacctatactattaactaccgcagatacgtatttacatctcggtaacatctgccagtcgcacaaatgcactaaggtgactgacagactgtagcgaccgatagagcgctgccttgttctagtcaaaattcaagcctggtggtcttctcgatccctctgcgcgcgccctgctcttttatcctgtgtgtctcctccacgagggaaggcgtcatgagaggacgacacgacgaccatgcagtgctgcgcaaccgcgcgcgtctgcctgttcactgagccccaggcggccggtccaagctattaccacgtcgcccccctccagggctgcgggcagcaacgtgtcgaccggtccgcctctgacgtgtccctcccgtcctctgcataaaagggtgcggctggcggggagcctcagcttcatatcgtcgcaagctctccgtgtttctctcgtttcttcactgctgccaactgtgctgtgcagtagttgcaagagctgattggtagctaggtacgctaggcaagtaggcatggctcccaagcggcgcggaaacaaggtggtgggctccgtggtgaagaccaaactagtgcaggagaccgtggaggtcatcgtcgccgacgacgatgggttgcacgcggagaagcagcaggtcccggaggctctggccctggcgcaccccaccgtggacgtctctggctccacggtggtgcatgtcgtcgaggtcactgccaaaagaggccgtggtggtggtggtggtggtggtggtggtggccggcgcaacgaggggaagccgccgccggaggaggattctgcagcagtacccgtgccgcagagccaggagacgcaggaccccaacgaggagctggagtttgagctggaggacgaggaggagaagcagccggagactccgcgcgtggcgtcggagaagaggaagaaagcagcaacaccaacaaagaagacgaagacgcagcagccgcggcggcggcggcagcggctggggcaggccagctccggaggcgacgccgggatgggcggcgtgggagggtacaggcggtacgtgtggcgcgtgctgaagcaggtgcacccggacctgggcgtgtcggggcacgccatgcaggtgctggacatgatgatggccgacatgttcgagcgcctcgcggaggaggcggcgcgcctgtccaaggccacgggcagggcgacgctcacctcccgcgaggtgcagagcgccgtcaggctcgtgctccccggggagctgggcaggcacgccatctctgagggcaccaaagccatctccaagtacatgtcctatgccgcctaggtagctaccgtacatgttttctgtaccttggaagtagtgcgtactgcagctgatgtattttcatgcatgccaactagactagctagtagttactgtacgtatgtagttgatagtgtaatcaggccgggtagcagctagctagaccaacttaattagctaatgtagtttgatgtacctgtctgtcagtctgtgtatgtttcgttgttttaatcgaccaacttgttccgtgtcctttttgttgtctagttctgtctactgttctgtttgtctgattggatccatcagcccatccatgtaccgcctggctagcttatcaggtattgcaatccaattcaggaactcacctctatctccttttctcagctttttctgtaaacaggatccgcacttttgctacaaaacaagttagattcactacagtaaactaacaaacatccgactgccgccttatctccgacgggtgtgtacgaggctgtcggaggcacttttgctacaaaacaagttagatt

SEQ ID NO: 78 - DNA, 2247 bp, Zea mays
tataaattagaacggaggggtatgtttttttacgtttccattatttgagtctgctcatcacgtttaatttgtttatacagttcaatgagttcagttacagcggatgataattcgacggcgtttatttgcatgcacttactttttacattacgcctagaatgtaatcggatggacgaatattacttgttttgtgtttattttcatatttttttctcttcggatacggatattatcgagttgtgccgaacaagattttgaatgtatatcgacatcttaaatatataactttgaatatacggatacgtatatagatcggatattgaatacccagctagactcagattaaattggatcttagttattttagggggtgtttgaatgcactaaaactaatagttagtggctaaaattagttgaaacatccaaacaccctatctaatagttcagctattagttatttttggaaaattagttaatagttaggtagttatctgttagctagctaattcaactaattataagttctagtgcatttaaataccccttagacggatcgaattagaacaccgatggataatatccataacattttacacctataaatatgtcctatatgtatatagatgtcaaaaacaggttgggcatgttggatgacccgtggcacgatacagttttaagcagtgccaaacatggcatggcggcgagctagacatgatacggtccggttttatttttgtatttttttttcgtaataatatctgtattatgcttgttcgaacctcacttgcttatgtggatgtgatagtatcaactcgctggcgttccgtgcatcgtaacatctatcacatcccacagatcatgtcgcttatcttttctgatactatagcagtagcacacagatgccggtcgaacccagaagcatcacgccatcaccccctccccttttcccaatcaaaaccacgtccgtcccaatggtaacccagcaccaatctcgtgttcgtgtgagacagagcatgcaaacaaacagcatgcatgcgcgaaggaaagcgtgcagcgcgcgggacgtgaaccgcgcgcgaaccctgccgcccgtccggtctcagctcacgcccagcgtccttcccaggccgcgatccggccatcctgacgcgcgtttcgtctgggccccaaaaccacgtccgccgcggcggcggcgcgcccacgggctcccgtgcccgccgatagtttgccggaccctgccgcctcctataaatgcagagccctccgccagcgtctcccgcatctcatcttcttcctccatccgcactactgagagcgacaaagctagctctctccagggatcggattcggatcaccatgccgtccaccgtgaactccgcgtccctcgaccagaaccttgagctcgccaagcaatgctcgcgaggtatacatacagatcaatcgatccgtttactgatgcttgcttgagcacataattaatcatgtcttcctgctgcagaggccgccctcgcagggttcaaggcagcagctgtcgcgaccgtcgcctctgcagttcccaccgtcagtgttcttcatctgtctcagcatgtaaacctcgctctaataatcctcgtcgcctgtatgtgattcgatcgatctcccctgttgttgtttcagctggttagcgtcaggatgctgccatgggccaaggccaacatcaaccccgctggccaggccctcatcgtctccacaggtcgatctctgaatctctccctgatccctctacgacgacctagctagctattactaccaagatgccttcacatgtctttctgtgtgtaactaatggcggaatgcctgctttaatttgctccacgaaaactgggtcgtcgtgcagttgctggcatggcgtacttcatcgcggcggacaagaagatcctctcgctggcgaggcagcactcgtacgagaacgcgccggagcacctcaaggacacctccttccagggcaccggccgtccgcacccagggttcttcaggccttgagcgagagaccatcaaactaccgccctagctagctggacgcactagttcgtcagcggccgccgattcatgtcgtccaagatcatatcaactactgctagcatatatatagtgtactagctagtagtaggcaccggcgatccgtgcatgcatgtgctcggagaaactgtaatggcttagtgctctgctccagtttactgtatctaccttccattatcatataataat

SEQ ID NO: 79 - DNA, 3000 bp, Zea mays
ctccccgtcgcaatgaggagactgtkgatctatttggttctcatctaactctatcacaacttgtccaagattatgcgactaaggcctcctctggtagggctcattcaacaactccttgaaaaaactaccaaaagggaggttttttatgggctctttctcgaaaagggaggaactcctcaaaaattcaacaggaaccgccggaactaaatttaagtaaaaaaaactaaagaaacaatgataagttacaccgaactataaagttcgttcgtgcgccacatgaccccacgtgtcccacgacctgaagggactcgtggcctacacacgcgcacaataatcagttaccatgaattattttatatgcgaatatcgacattacacatgaataatgtgaatttgaatttccgcagcatgcaaaattcccgtgagtttgaagggcgataaaaacgggtggccaaaattggggcggcaatgcaagaagataagcctatccatcccttgcattggtattgtcatctggggcagggcaggggagggcaggagaagctatctattggccatctatccagcagccgctctccccagccccactctctccatctagagctccctaactgcaccggccgccacaccaccgtaccccgacccctcgacaacaatggccaccgtcctaggcagcccccgcgcgccggccttcttcttctcgccgtcctccctccgtgccgcgccggcgcccaccgccgtggcgctgcctgcggccaaggtgggcatcatgggccgtagcgccagcagcaggggcaggctgcgcgcgcaggccacctacaacgtgaagctgatcacgccggagggggaggtggagctgcaggtgcccgacgacgtgtacatcctggaccaggccgaggaggacggcatcgacctgccctactcctgccgcgcggggtcctgctcctcctgcgccggcaaggtcgtctccggctccgtggaccagtccgaccagagctacctcgacgacggccagatcgccgccggctgggtgctcacctgccacgcctaccccacctctgacgtcgtcatcgagacgcacaaggaggaggagctcaccggcgcataatcattcatgcccatccatctctatctctgtgggtcgtcgttgtgtgtaatttgagtacgcgcgtacacgtactgcttttgtttcatttgagcactgttcgtgtaagcgtcgagttgccctgccatcgtctctctctctctcgatctctactatttctgtgtgtgcgcgcttctatttttccaatgttgagttacgcatatttgtccagtaaccttgttttgcttaatcacacacagctcccaaattccagtcgttctgtggtgacggttggttgtggggtgtggcggcgacgtcagtcatgggagaagagatccgaatgcatgggggtgttggcgctttctctgtccctctccctctccctgtcttctcgcgcgctcgccgtcgtcatggctcgacgaacagacattcactgccttccccgcgcgcttcagacagaccacgcttgtacgtagcgccgcttctctagtcgccggagcaggcacgagtgggtggcgaagaggccacggcccgacctgctagcgccagcgaccgcatctcgccaccagccgctgcccgctggccgctgccaccgtatgttgggttgggctgaagcctgaagcccgtgacctttttctttttctttattctttccttttcttttgccggacaagacaagtgatgttgcaaaatccatataaaatcctagagaaactataaagtttttttttgttgaactccttataacaacgtctaccctttaatatatggtatataatttcacatgttttagtataaattgtttactgtatttctatgtctatatgctcttggtaagattgtttataaattttaaatttaaaatcttaaaagtcataaacttatagaaaaaaattaggatcctaaacaattttaaacaaaaaaaaaaacttttcaattacaaaatcgtatgtgttctttatgcatgcatgcaactgagaagctgcccaacggccagcgcagcagaagggcagagcggtgggcggccacgtcgccgccgcaaaggaactgacgtgtcccggcccgacgacgacgacgcgaaccttcaccgcgccgcgctccacctctcttaagctaaatacgctagtgcaggcgcaggcatccagctgcgacaacgtagcatacactagtgcttttgctagctacttgtgcgagaagagagagcgcacggcgatagatatggcgcatagcaagcgcgaaatcaagcccgggagggcgcataccaaggtggccggcgacgacgagatgctgcggaccgggttcttcgacggcacgccgctggagggcggcaagatcgccgactcccagcccgtcgacctcttcgccgctgcccgccgcgtcgccgacgccccgtactcatcgcagcaggaaggaccacacgaggaggggaccaacaagaacgccgtcatcgcggagtcggagcccgtcgacctgcccgcgagcgcgcggggcgtggcggaggtgaaccgtgctgggccggaggagcagacaggccgcggtcggcagggcatggccgagcccaccgccggtggacgtcgcttgggattaggtcgcccggccccggcttaacgatccagatcaagcagtactcgactaccgccactacactacctgctctgctcgtgtcttgtttccagttttctttctctgtaagcggtgtagggtttgccaaacttttatgtacgaattatgaatatcaataaagtcgatctgctgctgcgtttcttggcaccaacaatctttccgcctcatcagagcaactcccccataaatttaaatttcctatagtgaatttgctagtctgctagcgacttactaagtagttcatactgaacgggagaacaagtgccatcgaggcttcatgcacatttcactga

SEQ ID NO: 80 - DNA, 1719 bp, Zea mays
gccatccttacgtcccgcggcttcccgactcttctagcaggaaccgagcgacctcggactcagcggctccgcctgtcggcgagcgcacgccgcgtgcctgcccgcggtctctcgtcgtctccagaagatgcgccccgatactcgtctttccacccttcttctacctccttccacccccttctccccacgccccatcggatcgcacgcgcaccgcaattgttgttcatcacctctccgaagaagcgtcacagagcaccgcccggaggttccccttcccgtcgccgtcgcttggccaggtaagtaatgactaatcgcctgagtctcctgcgcctcattacttcgaactgtggtttcgttagtcggacgtcggagcaatgctatgaaccttgccattccatgttacttggtgctgcgtgtccacattcgacgccacatttttttcttgctagccaaccattgcgcgtctgtgttacagttccaccggcgatatacattatgcgatcatttgatgtggatattggtgtttttttggttagcggatcaccttagttttgcactgaacatggaacaaatgtggatgttggtgtttttttgttagtggatcaccttagttttgcactgaagatggaacgaggaaatggggaaggggaactgaacgaagcacatagtttcagagactatgtgcactgcagagactatgcgaatggtcaagggaatttctcgccttggtttcctaatcctcgcttccttcttgtttcgaattcggtgtatatatgtggtttattcggcacgtttaggaatttagttggggaatggggcgcagtatgtcagggttgtgtcatctgaatatataatgcaagatctattactagtttaaattgacttattatgcaagtcgattaacctggctgggctcagtggtgtaccatggaagctttgtgattgtagccgggcagtagaacgctaaacctgaggtttggtactgtagcactcggctagtcagctgtatgccaagatgttacttactcgatactttctgccatgtctcatctgtgttaaccagggtgtgtgccacatagggcttctttccatgctgtttgttaacctgttcacacttcttccatatatatattcataatagctcgatctacaattttcctgtcttgagcttctttgttcctaaaaacgaattgtataccagcaatgtatatctgatgcaatattgtttgtaggatggctgaacaattctataccgtggcatcggatagtgaaaccaccggggaagacaaatcacaggcttcattccctgatgttgctgttggcattgacattgggacttccaaatgcagtgttgctatttggaatggccaccaagttgagctcttaaagaatacccgtaatcagaaagggatgaggtcatatgtcatgttcaaagatgatactctttcagcaggagttactggaggtgcagccaaggaaaatgcacacgaggaaagagacatcttgtcaggaagtgccatattcaacatgaaacgcctgataggaagaatggacactgatgaagtggttcaagctagtaagactctcccatttcttgtgcagacattgggtatcggtgtgagaccattcattgcagcgctcgtgaacaacatgtggcgatctacaactccggaggaggtccttgccatctttctacttgaactgaaggccttggtggag

SEQ ID NO: 81 - DNA, 3602 bp, Zea mays
ccattttaataattataatttagacaaaactaattaagtttatatatttatatatgtaatatatttgtatattattctaaatcatatgagagagatagttatatactacatttatgttatagaggcgcaagtagaagaatgtgttataagttgtacatcggaaaaatagcatgtaaatctatagaatcaattttcatctttcaccccatgaatttgaggtatgcttatatgataactttagaaagtggtggaatgtcatattctaaaaaaaatagcctatttcattagtaagattccaattcctcgaaatgaaagaaaacaaacggggccttattagaatggaattcaattccaatgatccaaatggggcgtaagggatttccactttcctaagaaaaattagctcattttcccttggtgtttaataattatgttttgttaaaaggtcagcttcccgagaatgcgttgtgtaaaagcccagtataagggtctgtttggttgggctgtggctgtgaaaaaagttgctgtgggctgtgagctgtggaaaaagctgctgtagactgtaagctgttaaaaagctaaaaaccgtttggtggaaaccactaaaagtcgttaaaaaatcttcgatatatgttttcacagttccatccgaaaagccactaaaagcaggtccagaggtgctttcagatttgcactacgagaaagtcggtttttagaaaaagctgcttcctggatccagccctttggktggcttttggctttwaggggcacaaaagccaaagccaaaagtcaaaccaaacacaccctaagtcgcgtgagtgggccatttatctattccctatgcaaatctcctgaaaaattacaacattagctcgttaaaaagtaaaataattttaaaaaattagagactaattatttgataactttatagttcatcacattttcattgaaaatgttttattttttttgccgaactgatctacacaatatttgaatctacacttttggcttgggctcggagaaaaatagtgcgtgcccagccctttgctctttccccgataacaacgcggtatgtggcctaacgcccaaatccggcccatttattccggtgcgtagatgcagacgcgggagcaggtgccaagtcactggctcactgctgcaggtggagtggtggacactagtgccgcggttcatctgacacgtgtcgccacgtgccgccatggcagcacctcagcccggccggcgggccgactgacgtcttgggcaaagcggcgagcgacgcaggcggcgaaagccatccgatttgacccctcgctagacctttcaagaacgaacgctgtgctgctcagatcagaccgtgtctgcctcaaagcgatgccaggacgccacgtccaagcaaagcacccgatgccattgccacctcccagcactcacgcgtgagcgtgactataaaaaacgcaccctctgcatccgcccccgtctgcctgccctaccgaatctttcgccgtcccatcagcccagcaattcttcgctgttcgaggacccctcggtttcgaccgaagcccagcaagccgaccacacaccgctgccgttggttccgtcccaagagatgggcaagtcctggtcgctcatcagccacctccacaccgtcgccgggtagtttcgctgctccctcgcagttcgttttccgattcctcctcgtccatctattgggctcgctcgacgctggttgcctgacgtgtgcgtttgctgcttctttgtcttgtctgtccctccctcccgtttcgcaggccaagcatcaccctgctgtaccctctgtaagttccttcatcaccctaataatagcagggaccagttttaccagtgcagcagtgaccgaccacggtccacggcatacgtgagctgagagcatcgtgctgggacatgcgtgccgttggtggtgctgcaggtacgcgtcggtgtgcgcgatggagagcccgtccaaggcggacgacgagcagtggctgtcatactggatcatacactccttcgtcaccctcctggagatgctcgccgagccgctcctccactgggtacccgtgtggtacccggccaaggtgctgttcgcggcgtggctggcgctgccgcagttcaagggcgcctccttcgtctacgagaagctcgtcagggaacagctccggaactaccgcgccagatacccgcgcaagggcgccgccgccgccgccgtcggcggcgacgacgacgaccataaggtgcacatagccaaggtaggtaaggatcggactagatgaagccgtcgcgttttgtcgttctagcgtatatgtgctgtagctgttaacccacctgtttcttcctgtcgggctgggaaacaaaacaaatgctaggctgaggctgagcacgaccatgtgcagtgaaagcgtgtggagacgacgcaggagctgaccggagatcatcacaggaacttctgtagatctggtgttccaaggtgttcattcgatgtatcatcctacgtatctctgtaactcttgtggactgccggtgtcgcgtaatgtatctgaactcttcgtgagtgcgatggaacccccctccccttgcagcacgagatgcttctgtaagaacgataacatgtagacaagccaagcgcctcagctccttaagtgaaattctctactactactactactataatgcaccagctttgtgcacggatgaattcatgcatcgtgcccatccccacccaaggactagattcaagtcaactacatgctatgcattacaacaagcatctgttgcttgcacaaatggataaatggcataaagggaataaaaattgggctagataaatggcataaagggaataaaaattgggctattgctatggtgcagaaacaagacagtactgcaatttgtctagatttagtgcctaaaaatatcgaagctgtggtcatagactaattctcttcccccttcgagatactatgatcacaccatgtccctattgcctgatatagtctagtgaagtccaaaccatatatactcaccttctcaaaaggagccatatatatatactcaccttctcaaaaggatacgagatatgtaggacacgatgagatcgaaaaacaagttcagtccatgtttgataatggcattatttcagtaagttgtcaaaaagtgttagttcatttgctaccctagtcgtattaggtgaaagaaaaaggatggcacatgaagattttgcatagactacatgaaattgaatgatgtaacagtatggaacaaatttcatatgctagttattgatgaatttaattcttggatgaactggtcggtgctaagttttttttagtatggaataaatttcatatgctagttattgatgaattcttggatgaactggccggtgctaagttcttttctaaactggatatggcattaggattaccctgatttcaccaaatcaggatgtcaatcatcagaattttaccaaaatcagtatgtcagtggatgagtataagatagcctttaaaacccatcgtggctagacccgtcatggccacttttcaatttcgagtaatgccttttgagttgaccaatgcgccaaa

SEQ ID NO: 82 - DNA, 2441 bp, Zea mays
tattacggttagtcaagactttacaggggaacctttggagacagacaaagaagtaagaacccaatgcacctgtgcaaagactgtactttccagtctttccctgaagaattatgacacatgattagaagacggttcaacctttcacacataaacacggaagatggctgtacctataaatactagagaacttggcataagggaattcgcatgacctcacaaaacctctcctgatggaattttagtttatatctggtggaatatttggaaggggaatcagataacctttcaacaacaggagctgcaaccgtcgaaagtagcccaactgatcagagataacaacccacagtttagcctggccatgcagcaaaccaccgactaaccttgagctttctgggctgtttttctacttctttggtaggttctgcactagagtttttgtttttattccgcttgtaagtttctctttctagtttctatcgtgtaatatatattatggcgaagcactcgccgttaattcaaaagaaagacaagcatctgaatttgaatccggcaacctatccaaattgtgccgtccaacgagactttgatatatatagaccactgaatgataacgggcgtaccagtcgtggactaacaaagacaggtaacatggcagcatggcgctcgccgcctgtttccgagaagaagagtatatgtcgttttctgatcagtagccgactgaccggttactgttcattgttcgctgttcattggcagcagcagagcagtggtagtagtactggtagtggtactctggtagctgatagcacgttactaataattatcatacgatttcctaagcggaatctcgagacgaaccacatttttcatccggtctatgagcgcgctcaatcatcagtgacagagaacactgtaggtccttaactggaactaacaattcagaaacaatcctaaatgtttattacactggatgaattttatttggatagatgatactaccgttcctgggctaaaaagcaaggtaaaaactggcctaacgcacgtgatcctcccatggtctcagaagaaaaccactcgcaaaaaataaaaaaatcgcagcaatgaggctgctttcagttgacggtgtgcacgactcgctgaacccagcgtgattttgtcccggttcttggagcccagcaaaaagggcgcctgaaattccacaggatgaaggggaaaacggcaacaaaagggactccgacatgcggtgcagaaagtgaaaacgcgagaaaagcgcccgtcctttctcgttcccttccgtgcagttgcaagtgttgccacgccgcgccgcgacacaccacgcaggaggcagcgagcgcaataatgcgcacgcagaggagcacagcggcgtcactgctgcggcagtaatggccgcctggttccacttccaccacggtgtgcactagcgctacaccgaggccaccttttcttggttccttcaacacgcctactccggcctccggatcgggcaccacgtctgccccctgctcccggaatctttgccaagcggcgccgctactgtccggcaaggcgaccccgtctctcggccggcgttgatctggtgatgcgcttttcacggtgttttttttttttgtttcactcccccctccgtcctgtgtgattgtgctgcagttttgtgccggtgctaacggaaaccggaaagtttgcgtctttatgttactgtggttgaggcgggcggagtggcggcgcgatgggcatctcggcgaggtggctcaagtcgttagttgggttgaggaaggtggggcggcagcagtagcagcggcgaaaggatgatgcagatgttggtcgaatggtacgcgccgcgccgagtttcttccaatcattatccagttctggcgtatcttgttcttaaattggtggttggtgcattcatttcgcttattgcctggacctatgtggattatgcaagaatcatttttcatacccgagtcaatccacaccaccaaaagaatgaactatttaatcaggcttcttgatcatatcctgggaaggtctgtactgatcaatgttagttatccatccttgactgaatcgggattgcagtcggtaattccttgtgcactggttgaattaattttaatttttttttactgaggagagcatattgttgagtaatttgttacctgctttgttcaaagcttacttaagtacttgacttcattggcacttcttgaactatgagaacaaaaaatcttggtggttatgcaatcacaagagctttaaattaactgatggatatgtatagcttagtatggccaaaccttgccttctgatatacacatggagaacatatttttgtgccacttccctcagactcagaatatacattgttttgcttacgagttgccaactggagaaactgcagaaaaccgatgtcgccgacc

SEQ ID NO: 83 - DNA, 2548 bp, Zea mays
tcaacagagagtgacaacaatcaacaggttaatggacatttttccagccatcgtgcttgagtgaatgtttttttgcgtgtgtgtaggtgtgcgtgtgtgtttaattgatataaaataacccaaccaaacatctcctaagatttccataagacagagaggtgaggaatagtattagttctcaaatgaattcaatcaactaaatatcaccatcatatcaacattaataatagaacaataaaattttcatgatagagaggctggtaaactttattttttcatttgattccagtgtagaaaatagtaatataagcagtaaattttttcttaaaaaagagtggaatattcacctttttatataaattagttattcttcgaagcgtcacacacggatgtcctaaagatattcttcaactgcaacacaagcattctgaaaaggttagtaacaatcagattaatgttgtaaaaatatgtgacacgaaaatttaaagaacacgaagtacaatacattggtaagaaatcaaagacacatattaaccaactattagagaacatatacatgtttcttctaatcctggttaggacataggacgagggtttcacttctatcacaatgtgctgcaactttcagagttaatcaaatctgttattcagtgttatgcagcgacaaattccaaatgtataatcccacgatgattcatgtgactccatttcgtctgtaattgatgcgaaactgccgaattgttactgctcccataagttaattgtaccacgcttgatgaagacgttcacgtatcttgtgttcgatttatgtagccgttattcagaccattgcagcgctgagcttcaaggcaattagttttgccccgcgatgtgaggcaggtgatgacacgcgcccttcgggccgatgccgacgcgccaccgtgcagactctaccgaggatgagagcagcgatcgagagacgtggagcgaggaggagactttaggagaggagagaccagagccttaagcggtgacacacccaatgatcaggaccacgcttgcacgtgtcaggtgcctctcctccacgcgtcgcgcgcatcacactgatactgtcatccagctcgacgctatataaagtggtaagcatcttcgtcctcggggccgccccaacggcccttgcgatcgccaccacgtcagcgcggaggatgcacggccaccagcagcagccgtacccatacgggctgcggcaagacccgctgcggcggatgcgaggtctggacgtgaaatcggcgctccaggccagcccgcgcacgtccacggtggccacggccgcactcgtcgtcccgctgggtgccgcgctgctgtgcgcgtcggggatcgcgctggctgcgaccgtgaccggtctcgcgctcgccacgcctcccctcgtgattttcagcccggtgctcatacccttcgcgctggccgcggggctcatcgcgtcggggctcctcgcgtcgggcgcgctcggcgtcgcgggcgtctcggcgctcacgtgggccgtcgggtacgtctggcaacggcagggcgaaggcggcggcggggtcactgggatggtggtgcagccgctggaccaggggtcgaagcgccatggtgtccaaggcgccgccgcgttcgtggggcaccgccgcctgccacgggacatcgacgttgctggtgaatgatggtttggcggcgtgtgtgcaaacttttctatgctagtttagtttgcaggtgtgtatggattttgatttatggcctggatggatcacactatgtagcgaacgatgtgttcaatttactgttggaataaaactgcagattggtgttacttaataataacgtcctagggaaaattgaaatactctcagcgtcctaaaaaactttatattctagaatttaataaatactgtatgatgtttttggagaatattgttttttacacaaaagactggaccttccagatttattagcgcgtaaaaataaaatttgctacctggcactctttgaaatttttgtctcaactgacgcactcagctcgcggtgttcagttacattcttgtgaagaaacattttctagctagaaaagtcaaatccccttccaatccctattccaatctattactcaaatccccttcaaatcggatccacgttgcggaatgggacgccttcctcccggccagccgcggggctctccacaggtcctgaaagttcgtcaggacagttgcatccagggggctagcccgactgtctctagcaatgttatatatatatatatatatatatatatatatatatatatatatatatatatatatatatatatatatatatatatatcctatattaggagcacttgacttccaataactaaaaggaagcccaggccagacagtgcaatccgacaggtctagacgtctataaaagaaaacccgcaatgctagggatcagtttttcacgccccatatcgattcattagcgatggcggttgacaggtataacggaagcctgagcttccaatagttaaagtaagctcaggccgaccacccct

SEQ ID NO: 84 - DNA, 2907 bp, Zea mays
cttctattttattctatattatatatatataattatatatatatttgcaaacattgttatacatcggtgatgcagatcttcgcttcgcatgggtggcaatttagtaacagaattgctctatgagttattgcttttagtttctgtctttccccaactgcgtttgagtttgcattgtcacatgtggccttttttggttgtaagacttgtttgatcggttgtggtgggctgtcatcctttggatgtaggccggaacctcttttcccattatctaaaaaaatacatcgtcatttccagtgagacccagcggtcgtgtcatcagaaaagcggacacgcgtcgcgtcgcgaggccaggtgggttatgcacggacctgctatgctagccatgctccaggctccagctccacccgtccaccgacttttcttctcaaccaggctgcctcctcaccatgcacgacgacacgtgcgatgccatcacacatccacactcacctgttgcctctatatccaccgccccaccgccgtagcaccagaaagaaatgtacagtctacacagctgaccagagagctagtgcaagcgatctagcaagttttaatcttggaagaagccagctaggcgcgtgaatatggatcgcctcactgtgatggcgccgccgctgctcgtcctcctcctgctgctcctctcgcggtgctcggcggcggcgtcacggcgcggcgggggctggtgggaggagggcgagggggagtggaggccgagcgaggaggaagagaagggcaaaggcaaagggagggggctgttcctgctgcaccgggtggagaaggtggtggagtcggagggcgggcaggtgcgcgtggtgcgcggccagccgtggccgccggcgtccttcgcctgccgcgaggggctcatgcacatcggcttcatcaccatggagcccaagacgctgttcgtgccgcagtacctcgactccagcatcactctcttcgtgcagcggggtacatgctgttattatttctatctatgcgcgtggcggaggctcgtgtcgttcccggtcgatatgagattgcaatcgcgtggtattgtgatgatgagcaggggaagcgaaggttgggtatattcacaaggacgagctcgtggagaggaagctcaagatgggcgacgtcctccacatcgacgccggctccaccttctacatggtcaaccccggcaaaggccagaggctgcagatcatatgcagcgtggacgcctccgatagccttggctttggacttccttatcaggtctcgccccctcctcttcctttttgcactctcgtcgtcttcctgttcttgttgcttgcaggcaagagaacatatagtatagtacatgcaatccgcgattcccagcactgatgaaccatgatccaacaaatccaatacaggccttcttcctcggcggcgcaggggacccggcgtcggtcattgccggttttgggccgaagacgcttacccgtgccttcaacgtgagatacatatactaatctaatatgaacatttcgcatggatcgtcgtgctctaattgacacaggtaggcggtagcagctagcagtacccataacttgcatgtcttctgctaggccacctacgacgagttggcgcgtatcctgttgccacgaaccggcggccccatcgtgtactacaccgcggacgcggagccagagagcggtgccgccgaagaggagcgcgggcaggtcgacgggcacgacggtgtgctggacaggggagcgcgacgcgagggcgccggcgcgtgggtgccgggtggcaggggagacggaggcgacgagtgcggcggcagcgatgacgcgcgtgaggcgacgtggtggtggacgaagctggttaacagggtcgtcggaggggcggctggcggcggcggcgctgcggaggcgaacaggaagggcaagaagaagaagggcggcgcgccggagccgtacaacctctacgacagcgagcccgggttccggaacgctacggctggaccgtctccgtcgacaagcaccagtacgagcccctcaagcacccggacatcggcgtctacctcgtgaacctcaccgcggtgcgcgccctgcctgaatcatatcgacctgcccgctttcttgttggattactttcgccgtttcgcgtcatggcagaggaggggttgctgctggtaagctcgtcgcaagcgcagccaagctgatcatcgttctgttcctccatctgcatggcaggggtcgatgctggcgccgcacgtgaaccctcgggcgacggagtacggcgtggtgctgggcggggaaggcacggtccaggtggtgttcccgaacgggtccctrgcgatgagcgaggtggtgcgccccggcgacgtgttctggatcccgcgctacttccccttctgccaggtggcggcgcgggccgggcccttcgagttcttcggcttcaccacctcggcgcgccgcaaccggccgcagttcctggtcggcgcctcctcggtgctccgtaccatgctagggccggagatcgccgccgcgttcggcgctcgcgagaaggagttcagtaagctggtgcgtgcgcaacgggaggccctgattatgccgtcctctcctgggaaggaggaggaggagcatgggaagaaagggagggagaaagaggagtcactgccgatggttgtcgagcaggcggcggcggagtgagctgcttgatctttcagtcttggactcttggcttcctcagaagcgcgcgcgcgccaccggaaagtgtttctactcttgttactgcgccttgtcagtgatcgtgttgttggttttttttttcgtggcgccgtctataaataaaagtgctttgtgcgtttacagctatgaaatcaggattatcatccgtaactcgtaagctggacgccaa

SEQ ID NO: 85 - DNA, 5105 bp, Zea mays
gccatcatcaccctaccccgagctgactcgggccgcagggacccagaccggtgtcccatctggctagctccgccagataggcaatgaaggcgccccgcatgctctgtgacgacggcggctctcagcctccttacggaagcaagaggacgtcagcaaggacccaaccgctccgacagctgtccctccgccaggctccatcgctcctccgacggccacgacatcgcaccagctgggtgccaaaatctctccggctgccacgacggcatgtacttagggcgctagctctcccccgctagacacgtagcactctgctacaccccccattgtacacctggatcctctccttacacctataaaaggaaggaccagggccctctcagagaaggttggccgcgcggggacgaggacgagacaggcgctcgcttggggccgctcgctccctctcccacgtggacgcttgtaaccccctactgcaagcgcacccgacctgggcgcgggacgaacacgaaggccgcgggattcccacctctctcacgccggtctccggccgcctcgctctccccccttcgcgctcgccctcgcgctcgatccatctaggctggggcacgcggcgacactcactcgtcggcccaagagacccccggtctcgaaacgccgacaatatgatatatagaaactataatgatacacatgcaattaactagtgctgataatatatttacactgtagatttttccgataagtagaaaccaaatatttcaccgccacaaaaggcttcatcccatcagttacccatgtagcaaaaccaaaatacttgtgtgcgagaggccaagagcccaaaccaccgccaaggaaacccccagcccaccaggcgccttcctctataaataccctaactccggtggccgaagttcctcacccattcactcactcatctcaaggcctaggtagcaccgtagcagctactagaagcagctagccagaacaactcgtccatggcgatggcctccgcggcttgctcatgcacggacggcacgtggtgggtgtacgcgctcccggcgctgctcggctccgacaccctgtgcgcccacccggccctcctggctggcctgatctttctggccaccgtctcggtggctctgctggcgtgggccacgtcgccgggcggtccggcgtggacgaacggccgcggccgcctcggcgtcactcctatcgtgggaccccgtggtctgcccgtgttcggcagcatcttcgcgctgtcccgcgggctgccgcaccgcgccctcgccgagatggcccgcgccgcagggccccgggccaaggagctcatggcgttctccgtcggtgacacgcccgcggtcgtgtcgtcctgcccggccacggcacgtgaggtgctcgcgcacccgtcattcgccgaccgccctgtgaagcggtcggcccgggagctcatgttcgcgcgtgccatcgggttcgcgcccaacggcgagtactggcgccgcctccgccgcgtcgcgtccacgcacctattctccccgcgccgggtcgcctcgcacgagccgggacgccaaggtgacgcggaggccatgctccgctccatcgccgccgaacagtcggcctctggcgccgtcgccctccgcccgcacctccaggccgccgctctcaacaacatcatgggcagcgtcttcggcacgcggtacgacgtcacatcaggcgccggcgccgcggaggccgagcatctcaagagcatggtgcgcgaggggttcgagctcctcggcgccttcaactggtccgaccacctcccctggctcgcccacctgtacgacccaagcaacgtcacccgccggtgcgccgcgctcgtgccgcgcgtccagaccttcgtccgtggcgtcatcgacgagcaccggcgccgccgccaaaactccgccgccctcaacgacaatgctgacttcgtcgacgtgctcctctccctcgagggtgacgagaagctcggcgacgacgacatggtcgccatcctctgggtaaagttcaaatcgatcgctttcctagcttgtttaactgcgcatacttctcagttctcaactgcgcatacctgtcggttctacagttttgtgtcgggctgtcggttgttcccggaagggaaaaaaaagaacaaagctctgtcgctgaaaaaaacatactgtacatgcatataatttgtttttgcaggagatggtcttccgcggtacggacacgacggcgcttctgaccgagtggtgcatggcggagctggtgcgccacccggcggtgcaggcgagggtgcgcgccgaggtcgacgcggctgtcggtgccggaggttgccccaccgacgccgacgtggcgcgcatgccgtacctgcaggcggttgtgaaggagacgctgcgcgcccacccgcctggcccgctgctgagctgggctcgcctcgccaccgccgacgtgccactctgcaacggcatggtggtcccggctggcaccacggcgatggtgaatatgtgggccataacccacgatgccgccgtgtgggccgacccggacgcgttcgcgccggagcggttcctgccctccgagggcggcgccgacgtggacgtccgcggcgtcgacctccgcctggccccgttcggcgccgggcgtcgcgtctgccccggcaagaacctgggcctcaccaccgtgggcctctgggttgcccgcctcgtgcacgccttccagtgggccctgcctgacggcgcggcggccgtttgcctcgacgaggtcctcaagctctccctggagatgaagacgccgctcgtcgccgcagccatcccccgcaccgcctgatccgtcctgccgccgacgcgtcacgtcacgcgttgtttgcatggatgatggtatctttgtctgtctgtgtggtcttcgctaaagtttgcttcttctcgatcgtcggttcgttcgtgcctccaccttagcctagggtttggtttcttgcaaggtagtgagtgtgtcttagtctcaccatcaccggggctccaattttggaaagctgcgtgttaggagttaacccctagacatgtttgcgtcttgatcgccaccacccatcagtatcagcgcagaaactacatatagatcagtgtttgtcgaccagtcatggaagtcgtgtgctctcaagtctgatgtattatatacatatatatgtattgtaatgtgattatcaagaaccgtgctatttacatattgctgtttaattgaactaattaactaaatgtcccttatatatatgtccagatcggccactaattaactagttattttacaaatatttacaagtcggtcgtcttaagaaatcaacgcacttcaacagtattgagccgtctggtgtcaaatgagacttgcgttcactaaagaacttaatcaggttctttttgaagcacgtatttcgattgcctgctgctggctgtaatatatcaattcaagcacttaaacaggttctttttcagtgcaacaagggatgtagaggtgccatggtttgggtaagcactcatttctgtggtttatagttcgtgagtagttccttatgtgtttgcctgcccctggttgtaatatatgataatgcagaacaatctcttgcaatactatagggccctccattctaatttatagattgttttggcctagtcgattaactgttcatgaacatctttattggatggactaaccaacgtcggcctaagtctaggggaaaaggcccaaccagcgccatgagtagatcagcctgcaccagcgtgaacagtgaaggcaggcccaaccagcgccagcatcagatcagcccgcaccagcgcgaacagtagacgagtgaatagtaaccgagtgaacgaaggcaagagctaaataaatatgacagtttctatatttgtatatttatcttaggagtcaagttgattagtttctatatccattggattagagtttaggtaagttaaatatgtaacttgaggaccaaattgatgcctgccaaggctatataaggacctagattatactctcttaaattgagtttcaatccaatctgatgacaaaatcacggcacagaccaccgaggtgtcctccctgaaagtcgaccaggttgtttgtattcacatcgccgtcaacaatatgcagtcccaaacaagtggatggtaagcacaggctcactacggaccacatcgcctctgccagcaacacctacgcaaggtacaaggttgtcgtcaccttcatcaacagtactaaccacaagctatgcttttcccaagtaggacggcatgagaataatggcgatgcgaggagagggctctacgggctcactaaggtggaaatgagaagaagaaaacgagaagggcatgaaaaggtcaaagttatatggcgacactttaaatgagaactgaaaaacacctttaatacaagttcttagacgtgtgagcgtgaactccaaagggaaattgaaagtgaaattggtactaaaaacctttagggtcgatttggtcacaaggggctcacgggggattggaggggattgagtgaaaaataaactaattttcctcttaatatccttcaatcgccccgtgattcatggacaccaaatcagctcttaaagagaaatttaaagtgaacatttagtatcggttcatgaaaaaactgataccatagggggtctccagagagatgtcagactgaaagaggggtgttctttagagagattagagagtaaacaaagcaaataagtaattcatcttgaagattacggtgccaggctcctctatgttcttggcaccggcttaaggggagtgagtgtttacaaagtaggtacatcagcatttgttgaagacaccaactgagatgctgatgttaagttatcggtaccggttagagacaccaaccaacttcaaccgatatcgacaacttaatactccctccgtttcgttttagttgtcgctggatagtgtaaaattgaactatccagcgacaactaaaaaaacggagagagtatcagtattagttgatatcttcaagcgacactaatgctctctacgggatcgcgatggttaaaactgatactgacacgtacttgagctagtatcggt

SEQ ID NO: 86 - DNA, 4000 bp, Zea mays
cttccgataaaaatatttggaaccgcatcctgcatatttatttagaaacattgcatcgataaccgttgcagagcaatatacagggtactcaccaactaaatccataccatacagtgacaggtgaaattggaatacttgatcagcttgaataacagagagcaaagataaatctagccctggtaataaccatggctcacatcttatgtacaagagccaattctaaagacacagctgcgggatgtagcagttaggcaaggaaagaaacaaaattaccagctaactgctaagtgtcaaaacattgctttacactaaagggttaaatatttgccaagaaggtagtgggcacctctcctctgcaagcagcagcataatctagggcgaggcttggtgcagcttccttgtaggggcatatgaacttatccatgaaggcactctcactcatctggacagcagtatagtagctccggtcctgctccaacaatccattttccttcacgcctagtttggatcactgtaattgaattccattctaataatagtaatttagacatatatcaattaagataattcacttttgtacaaaatatatttgtatactattattagcaatatatcctaaatatttatgtgctacatttttactatagaggagtagaacgaagagtgtcatataagttatagattagaaacaaattctaatcatgcataaaatcatttcccatcctccaccctatgaatttgagataggcttatatctgaactttagaaagtggtggaatgtcaaattccaaactaaataagttactttattgagtgaattctaattcttttgatttgaaggaatccaaacgccccgtcaggtaattcacaacatagtgtcggggcatgaggcggcgttccaagctatatgtaagcagagcaggcctgtgagcaatgtactctggctccaacccaaccttggtgatcaggaacgcggatccgggcgtcgccgggggcgcgaccctgtgcttgccaaattgaaaatttagcaaagtattttttttaccttgttgattatgttggataaccgcatgtccctaatttgaatgatattgctccttatctctaaacgaccaactgtagcttgttctattgaaaactcatcagaataatccatagagataacatcaccaacaaggactgaatcatccagagatgcatcaatggccattggagctagggaagtcggcctaactaccttcaacccgacttggccaacttgtacagtagaagacaccatggaaatcgatttaacggactctaagggagtgtttgaatacactaaaactaatagttttagtggctaaaattagttggagacatccaaacaccctagctaatattttagctattaactatttttagtaaattagttacaagttagctagctatttattagctagctaattctactaacaaatttttagctaactaacgcattcaaacactgtctaaacaggtaatctaggcacgcgaagtctgactggccagcccaaactcagcctttgtagtagagagagccgaccttggcggcctgcacgacatttttgaactttttttatcggtttggtcttcattactttatattctttgtcaaccaaatactcccttcctcttaaattataattcgtttgattttttttatgtaaagttttatgtatagtccacctagttaactttaaaagttggttagtctagattttatggaggccgactacaatagcaaatctaacaataaaaaagttagctagatcagatttcaagaacagtctgaccgattttgaagatttgattcaggttaattctacgattttgaagatccgacctaacctaattttacgttcagactaaagacatgtttgtttaagattatgatatgtctagttatataatttaacttattttaaactaacacttaatttaaaataagttagattatatgatcagaatattatatttatataattccaaacaaataactctaatatcgattctggtttatattgtataatttttatgttgaatattacggcgcgccaacaaattgcgatcgttatgtaatcatgtatacgaccgactgtactattcataaaatataattcaaaataggagatgctcgagatagcctggcaagggaggaggtatcggaggcggcgtcgcctgccgcgagcgcaccatgcgtcaacgctcaggacctctcgcaggcgcgctgttggatcgtgagcaagcacgcgtggcaaatggcatgcattcctctcgtgcaagtgccgagctgagggtcccgacgcggggggcaggacgtggcagagcgccgcccaggcggacgccaagtgccgagctaccagggcaccttctttattccgtccttgctcagtcacacctcgctctcgctcactctcgccgtccgcacagccgctcatcgtctcccactgcctgccctctccctgcgccggccggtgccccgacgcgccatgtcgtacaacaaggccccgtccatcaccgccgagaccataaatcccagggtttcttcttcagaaatcatatatatgttcatccattgcattcttccgatttaaactgaaaagcttaggtgacgtacgttgtcccttcgtttgaaggtcaagatcatcagatacgcgccttgcggggagatcgtcaagcacgcagcggtaaattgcaccactgctgttaattcatgctgctttacgacgacccctcgcaccttttttttttgcaaaattacgatgactaataggaggacaaaaaatattgcctttcgctatgtgtctcgttcagcggctggagcaggagatagaggagaaccctgcttctatccctttccaagaggtgaaaactcctcccctgtgcagcaagcaagtgcgtgcgtgcgtgtatcacaatctatatatcacctaacttgaccagccttccatgctgtccaacagataatatactgcaacctcgggaacccccaggtccttggtcagccgccactaactttctttcgtgaggttaccacccttaattaccctcagaaacacaaaagaacaagacaacgcaagtgtgtttgatggtcgaaattgctggctccaggttctctccctgtgcgacaacccagccctcatggacagagatgaagctcgtgccttattcaggttggactatgcagctgagctcgctttactgtttactgtactattacctccgtcctttttttttatttgtcgtgttttagttaaaataaactaacggacgacaaatattcgagaaagtagcttcataagctgagtatgatacatagtttggcagaaacttgactctgacaatgtttccatctactgtgtcgcagcccttgtagcataagaagggccaggaggatcctaaactcgatccccagcaaagacactggtggatatactgattgccgggtacccaactgaaactgcttagctttcttataaataaacaaacactgggatggtgtatattttccccttcacacttactggttacatcgttcaatcacacttgcttggtgtgatcagggaatcaaatgcctgcgtcaagtagtagcagatgggattaccgcaagagacggttttccatcgacagcggacgatatctttctgacagatggagccacctcagcggtaagactctgcatttcagaacctacatctacatgacgcagcacagatggaactggatcctttacatacagatacgaacatcagtgtcaataccttcagctcgaccgcggctcctttctgccccttttttgcagatcaacttgatgatgcagatactcatcaggtctcacgaagacggcatcttgagccctctacccgaataccccttgtactcggcctccatcatactgcacggtggaacgatggtacagcactagtgtagttcaaaaagatagccaagcaatatctgcgaaacgcagtagaagtggctt

SEQ ID NO: 87 - DNA, 2865 bp, Zea mays
tcatcggtactcgcgatgtcgtgcgcgtcgacgactactatatcaaggactatgacgtcatcttgacaccagaggtgacacagaggaggaccaacaacaataaccactcgccaccgtcggtcggtctgcctttaattctcttcgcaaacacatacacttgcttttttgtttaagcaagcgtgtatgatggtgcgtacctgatgactgacaatgtacgagggaacttctaccggcaggtgtaacacgtgctcttcaatgataagatcatgcaaatcgtgtcatatatcgaagcctgcatgatactgatggttgaaatatagtagtgaaacagctaaattaaaataacaaaatttatgtatggctaggattacaaataaattataaaatatttttttataacaatataagacacattttgtatataagttattatattattatatgtttctgttgcaacgcacgagtactcacctagtatatccataggcttttgatcctctacgtcgagtttttgcttgcatgagagtcgacctcttctcttttatcccttcttctctatcttagcatttgctcgatcaactgtaaactaattattatacgtacttcaagtcatattgaaaccaatggcatttaaaaaagtaaccatctgaatggaaatatcaagctaaataaagaagttacgggagtatgtgctgagaaaacctagacacattcagagaagttaccaagcaagccttaaataacaatttttggtgctttattgcagatagatactcctataggttggagagaagggcggcgatgctaatgcaccatgcatgtgtgtctgcccatatactctatcttatctgaatcctaccaaactctttaatttgactctttatgaatggaattcccatatgaattctataacacaacagtacgacgtctactggagcatactgccaagcacgctagccgacggctcggcgaccgtctgaggctgtccatgcatgcaaatgtgcacacatgtacgccccttttagcgctggcatgcacccacgaccacgacacggctcccgtggccgtgaatctgacatcccgggactcccgcctccggccgcgcgtgccgtgtcagcctccatcgagcggtgtactccatgcaagcctcgacccgggccacgtacccccctcccctccgctacgtgtcaccgctctcgtcacctatatatggcggtcttgtccagtctttccacttaactactccgtcactttgtttgcaaagctcctcctcgatccatcgatcactgcaccggccggcggcaccgcgctcgcaggggctagccaacgagacggcagcaatggcggaccgtgaccgcagcggcatctacggcggcgcccacgccacctacgggcagcagcagcagcagggaggaggcgggcgcccgatgggtgagcaggtgaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcaccgcaggatggcggaggccgcggcgcacgcgggccacaagaccgcgcaggcaggccaggccatccagggcagggcgcaggaggccggcgccgggggaggtgcaggtgccggcgctggcggcggcggcagggcttcctcgtaagcaagtcatccatgcatggattatggatagatgcgcgcgtgcgtgtctatcagtatcagcagccagcagggtcgtcgcggaatgctgtgttcctgtacgtgtgggtgaccgtccttccgtccttcgtctttctccccccgagtgtgtgttacgtatgtcctggtgttcgtcgtgtgtgttcatcgccgctccagttgaattccggtgtctgttcatcgccgctccaggtcgtagatgtgaatatactttgctaggggaataagtgataagtctgtctggaaggtaatgtttgagctttgctagtgtggctgggcactctggtcactggttgtgttgtgcatgcatcagctgtatgatcgtcgtctgttgtggaaaattggtcaatgtattctcttgctgaataatttgtgacatctaattgttatgtatcgtctctttgctgaataatcagtttctgatttatcttgcattattttgagaatgaaatgatgatggtagctcagtctgcagctattcgggcgcacactgggtggctcactgtttggccacccagttacttacgtttagtgttgtggcccactgttttgccacccagttacttgaacgttgttttatcgtaaatttgagtattattaagagaaacattcaaaagaaaatcatctccaacgaactctatgaatgcctctttaaattttattcatcaacctttgctctatacttttaggctatttattcctcactctctagttttatatagcctacttcatttttatatagcttacttcactcgccttggattagtctacttttctctcgtggtagagagtaacaaaattaataaatttggttaatcttatgagctgagttgctctatttcatctcctatttattttctatttcatcttctatttattttctttttaagcttactacaaaaatagtgtcatctctcttatttattttt

SEQ ID NO: 88 - DNA, 3425 bp, Zea mays
ctcacacaaatctaaatagtaaagtagatctggttacagagcagaaatttgaaagtgtcatagataggctgaaaatatgtagtcacgagcaacacattactacttaaaggccatcaatactgttttttcttctccaacctatgtaactatgttattcttttcctccttaaatgtaaggcaaacctcctatcctttttctttcaaattcattgcattaactgagtggttcttacatgtagaaatgaagttggagcaaagaggcttggtgcggaaccgaaaatttgttagtctataaatcattagagggcgcctgaagttcgccaaatgttctagtagattttagtaaccacccacgctagctgactcgaccaccgtctgctggctggcgctgtccatgcacatgtacagcccctggcgctggcacgcacccacggcatacctcccatgactctgacagcccgggactccctccgctcgtgccgtgtcagcctcactcgagcggtgtactccatgcaarcctccacctcggccacgtaccccctccccttccttacgtgtcaccgctctcgctccctatatacggctgcctggccaggcctttccacttcactactctctctctctgcctgcaaagctgcttgatccatcatcgatcacatcactccaccagccgcagcgcacgaacgtacgactcatggcagatcgcgaccgcagcggcatctacggcggcggcgcctacgggcagcagcaggggggcggccgcccgatgggtgagcaggtgaagggcatgatccacgacaaggggccgacggcgtcccaggcgctgacggtggcgacgctgttccctctgggcgggctgctcctggtgctgtcgggcctggcgctggcggcctccacggtggggctggccctggccacgcccgtgttcctgctcttcagccccgtgctcgtccccgccgcgctgctcatcggcacggccgtcgcggggttcctcacgtcgggcgcgctggggctcggcggcctgtcctcgctcacgtgcctcgccaacacggcgcggcaggcgttccagcgcaccccggactacgtcgaggaggcgcgccgcaggatggcggaggccgcggcgcacgcgggccacaagaccgcgcaggccgggcacggcatccagagcaaggcgcaggaggccggtgctggcactggcgccggcggcggcaggacttcctcgtaaataagcaaatcatccatggatgcgtgcgtgtcaatgtcagcagggtcgtcgtggaatgctcttctctgtacgtgggtaaccgtcttttggtcgtcttccccgagtgcgtgataaatatcctggtgttcgtcgtgtgtggtgctgccctaggctggggcctggggtcggctgtgtaccggtatctgttcatcgccgctccgtcgcgttcggagatgtgaataagtgatgtgaaggttaagtttcagctttgctattgctattgtggcttgtgcatcagcggtttgctcgttgactgttatggaaataagcattggtgaaggtattgtctattatttgctgaataattttgttatatatgataaaattgagtttactttttatggaactttgcaaacttaatgctaactaaaaacaagtatttacacagaagattcaatatccttcaatatcctatttattcacgataaaaccaaaaaactaatattcaaataagtatccgttttttatctctgatgccaaagctgcgaggaaaacccatatttctttgctgcagacacgactgctgagctttttttaaaatttccaatgctctaattgcagtatacagagcatgcgtgcgaactgcaatttgtgcaagagcttggaacctgtgtaaatttttgactgtagcagaaaaatcacacaaattatacggattgccatttcgttgtaaatagtcctgcaattgaataaaaaatgttgctgattgttgtaaattcagagatgtacaaacgttgccgttccactgcatccagtaggtgatcgctggtcatacgaattttacacttaaaccacagatccaaaacctgttctccaaaayaatctatgtttcagacggcagaagacattcaaacaaatttgcaaaaagttattcagatggcaccaaatgtgaattcagcaataacatcatctgtgttttcccctgcaaaattaaaagttcagaaaaaaacagtgtatggttcagacttcagaggaacagtctattactttgaatattaccatgaatagctgagagaaggtgcataattcagacatttcttccttgtaattgaagaaatagtttgtattgtcgcacgcttccacatgaagttaaactcactgctctatctacctaaccaactcagctaataatcccttatctttaagcattccccttttcttatcttccttcttttcaaccttatacttttttgttatgtcaaattctattgcgcttaaaacttttttgtttgcacctctttttttcttcgggcttatttcgttagagcatctccaatcgtgttttaaatcaatttcttatcttgaaatatatgacatattttataatttttatcaaaaaaaaatgatacaccattaagtaactcaaatatactataccgcggactagtatccttgttttctacaccattattatcattcctaataatataatttactttttaaaatatatgatttatggtttaattgttggagcacaatttgtttttagtgtactaaacaatttaaataatgtaatattttgatttttggacgctattttgagtcaccttgttggctctttatttaaaattaaatagggagagatctaattcttctcattcctctytaatactctccaaccgaataaagccttaggattgttgtattcgaagtcaatagggggagcaacacctgtgggaacaatgaycggatccatggcactgagaaaactactcatttcttcttcggcttgcttactaagcatgtaagtgtgccgtttattaataataagttacacactagatgaaaacagggatggtaacgggaaattctttgtcagggaatagttcaccattcccgtccccgcgataaatttgtttccacgcgcatctacacgaacgtttgcggggacgtttctttatcatccccgtttcccgcggggataaatcaaattataattaagaagtatgtactccgttattaatacaaaacattgtcacttatgctcatcatcatatgtaagaaattattatgagtacatcaaattaagcatatttatacaataaatgatctattgataagataattatttatttttatcattaactattacacgaaacatcatcacgtgtaaaatttaacggggtctgcgaggaacaggaatgggtaatgcttcctcaac

SEQ ID NO: 89 - DNA, 5254 bp, Zea mays
tctttaggatcaatgtcatccaatgtttatgatgccagaagtaacatgattggagagatcgagaacagatcaacattcctcttagctgtaagtattttttgccggcaccacctggcacaagaatcctgccaaatttatgagttccactattaatacataattataactagtaaccaaattttaataatgcaaagtgacaaatgttttataaaaacatggaatgctccaggtaaaagctgatgtggagacacaaggagaatttgttgagtctctagcaaatgaggtccgagcagcaagctttgtaaatattgatgatgttgttgcatttgtaaattggttggatgaggagttatctttcttggtaagcagcccgaacaggccctccatactacacctttgttacaaagtaccagttattgtacttatgacacattttcacattttaggttgatgagcgagcagtgttaaagcattttgattggccagagagcaaaacagatgcaataagagaagcagcttttgaataccaggatctgataaagttacagaacaaggtttcatcctttaccgatgatccacaacttgcatgtgaataagctctcaagaagatgtattctttgcttgaaaagtaagtgtctcatcagacctagtattctctctagcatagtccttcagagtgaaacagcaacatttttgcctggttcaagatatgtatgtgatgaacataattttactcaaaagtgatggtttcattgtactaactagcatcaatacttatgccagagtggagcagtgtgtctatgcactactccgtacaagagacatgtccctcacgctacaaggagtatgggatcccagttgactggttgtctgattccggagtagttggcaaggtgtgcatctctagcatacactccaatttcgccaaacatgtgttaaaaacatgcatttttaattccttatgaaggtgggtatgattttactcagagtatgcacctaaatgaactcaataatttatttgcatgttggatatatcaaattggcatctgttcagcttgcgaacaagtatatgaagagggttgcatcagaacttgatgcgttggaaggcactgagaaagagcccaatagagagtttttgcttctccagggtggcagggtgtgaggttctctttccgtgttcatcaggtaaaatcctcacgccaatattttcagaacccaggtacttcacaggtgatatgaatatttcgaatcagtaacttattcatctaccttctgcagtttgctgggggatttgatgcagaaagcatgaaagcttttgaagagctaagaagcaagatgagtacacataaatctgctccacagatatctgaaggctgaagcatgagttgaacacaggattcaacacacatctggtcttacttgctggatactgatttttagttgtgtacaaaagagaagtttaaattccagtgtggtgtcaagcaagttgtatatgctaagagaatacaagtcatcaggttatgttcatttattttttatgaaagtaagggcaggacaggttatgttcatgcatataacagttttagctcaagtgtataccttttcattttaaaggctgtaactgtaaacaggcagctccagaaaatgaatcacttgggcatgtttttgttgctacgcatatactactaataaaatgtcaaaagcgtccattcggttagtccggtacccatggagataattcgtctgaatgaaccttgccgtctttgttaaaagaaaaaaaaattactttgtgcctcaccgcattcgatactttgaagaaattgacccgttaccagttaccacccacgcacatacagtgctagaacagcagatctgacttgaagaagcatatatcaagacaaaagtccacctgaacggagacagtcgaggttgtggcggagagctccacctgaacgccgaagtggaaccgaacagttccagtggttggccgtggcgagctggccacctactagtctccggggagggggctggctccagcgcctccgagcgcggtgccagtgtcgccgtcgcgtgggttccggttccggccgccgccgcttgccggggaatccaaacggcagacggggtcgccgtgaccgtgaatcgtgtttacttgggccttatctaggccacaacgtttgtgactctgtgagcttattgtttgggcccatctctttggcgtgtctgagaacgaattttatcatcaagcccacgaagccaaaccaggatcctacccacctagcacatactagtgaagaaggtaaacaaagtgtattttcatgttaattcacatgcgcgtggtcgacaccacaccacacgacttaaaactcggtgttatgttctacgaccacaagagccatgttaattttaagccgtgtctgcgccatgatccgaagagcgcgtacagtgagagaagaaacgcacggccgccagcagctgccgcgggagcgaggcagcacgtccgttggtcggcccgggcgggagcgaggcacacgtcgcgcatgcacgcgcggcgcccggacacgtgccggcgcgggcacgtccctctccgcgccttatactcctccccgccatttcccagcagccacaacaagacagagagatacccgatcgccaccgccgaccggactcccctcaaaggcggccacagcacagcgagcatgggagaccggcatcagcatgggcctggcagcggcgtggaggacccggcgacgctgctgcggcgggtgcagacccgcgcgcccaactcgacgcaggtggtggggttcctgacgctgctcgtctctggcgccgtgctgctgctgctgacgggggtgacgctgacgggcgccgtggtggcgctcgtcttcctgggccccatcgcgctgctcaccagccccatctgggtgcccgtcgccgtcgtgatctccgccatcgtcgccgccgcgctctccgcctgcgccttcgccgccgccgcgctccccgtggccacctggatgtaccgctacttcacgggccgccaccccgtgggcgccgaccgggtggactacgcgcgcagccggatcgccgacaccgccagccacgtcaaggactacgcgcgcgagtacggcggatacctgcgcacccgcgccaaggacgccgcgcccggcgcgtaggcgcgcgccgcccggcgtgcatgtgctgcaacccccgtccgtggtgccgctagctataggtcatcattgctgtcgtcgtcgtcgtcttagttcctcttgcattgcgtgtgcgtactctgctccaatcatcccccgcccgtgttgtcaagcaatttctggtcctcttgcttgctttctctaggagatgctgttcatttcgatctcttggcgcaatttgatctttcgcttggtgtaattaggtcattagttaaatcataagagactcgtgttcatggtctttctcacaaccttaaaacaactaattgttgttgaaaagaatatatataggtaaccttgctcagttttcgtcgaacagaatgatggcaacctttcttctgcaaacagttgcagataatgcctaggatttttgttctctgtgctgaatcatctatacaatccgacgtatgtactcacctcccgaggttgctccatcccaaaactggagttggcatagcgtggtccccgtacgatgcataacaagtgtcatacagaaggttgtgtcatgtgtgtatctagtgtgccacatgctataatctctgtagtcttttaattaccctttggggtgttaaagggcgatcagagtgacatcagtgatctaaggtacgtagaccgcataaatggtcgtgtctcgtgaacccaagcacgacttattatggacctcagactgacttagtatggttattgtggtgacatgcagtacatgtgctgagctaaaatgctcgaccaatatagtccgacctatttaattagttgtaccctgtcactacatgtaaattttctcatccagtcttccccgtcccgtcgttgctcctgaatagagttagtaatagaataatagatcacaacccgaatatttcctcacaaattgattgagtccttaattaattctagcttaaaacttaattaattctagcttacaagtccgatccttaaattttattactagtcctacttcgagtaatcaccgtatacatgtactcgcgtatttaacagtcattccaggacatcacacatgttctgagtgcgaacatatgcaacagatcatgtttagtcgtttcaaacacgtgaacgcgcccgcaaaaacatacacactcgcgaggcaattaagtcgttgaccattaccggacagacatgttccatttcgctacggcattttactttgaccatccatccgatgatccaatccaagatgctgtcttggacccgaccacgtacacaacaaccctgattccctgaagccttccgcccctctttctgacttgggtttagtttcgcagcagcagtgaaagtgccccgcccaaagaaaccagccgcgcacgtacttgcattgcataagcataaatgaagggtccggccggcgccatagccatatataccgagcctgaatttgaaacccccctcccatggaggctgaggcgttcccaatacggtccacgcgggctgtccgtcgccgcaagtaccaccgcctgggccagctgacggacgtcgacgacggcaccgagctccgggcgcggcagctggtggggcgcctgcgctccggcggcggcgcacgcttgcggcggcgggtgcgcgtgctcctggcgggcccgcggcgtgcgctggcacgggcgcgggacgcgtgcgtacgcggcatgctggctctggccaggcgggcgtcggcactcgcgctgccctgcggcgccgtcgcgaagcagcagcgggtcgcggcgcccggcaagcccaccgagttcgagcggcgcctcgtcttggagatgtacaagtccatcgtcgcgtccaaggagctcaccgccatgctccactcctccgccgcgcacctgccgcccaagagtacgcccgctgcggggatcccgtcgtccacgcatctgctcgacatgtgatccaccatttcaccgccgatggaggaggaaaacgtacgagctgattcgcttaatcactgctagagaaattaattcagaggatcgtcccaagctgttcagcagcgtcgtcgttatatacgacagtagctaattttatgatgcatgtgcacatgtcatgcatccacg

SEQ ID NO: 90 - DNA, 3177 bp, Zea mays
atctcacggtgtatattttggagattgcatttctctatatatatatgactatacttcgttttgtgacatgtttttactgtttccgtagtatatactcatgagacgttctctcacgtatatattatacatatggcacgttgcttgattattttggactgtcgttccattattgagatgttcggtagcttagtcttccttcccaaccctattggcctatatatttggatggaattgaaattgtgaatctttgtattaaatattttttttcatgatatgtggatttatttggatggagtggaaattgtgaatctttgctagatacgtgttttgtctatacgcgttactctaaagggttatcagtgacgtgttttgtctatacgtgtcactactaaagggacatcaattagacaattacacatttttacatgccaataatgtgtccatattagaggcacaatatgagtgattattttccacatcggtaatgatatgatagtctagatgtgttataaggaaaatagaccgaggtccattaataattaacgttttggtatttgatgatcaacataaccatttgcactagtgttatttgttggtgtttttgttatagttcaaatgatggaatgtgaacttggacaaagacaaagtggtgatcgagatgatcaatacctcaagaaagatattagaaggctcgttgaagatatacaataacatgcaagagttcaagataaaactaagccagatgtgcagatcatgaagaaacaaagcaagaaaaaggttgaggagcgtcagaccaaggggttggaccatcccaagggagacatcagattatacggtagaataaaatcctaactagcaacaaacataagcattagaccctagaagttggactatatgatcgcactattcatctcatagagacataagactgtaactaccttgaaccatctcaaacagagtatgatggagacagaaaggttccaaagaggagaaaggggtgtcacattagtgtgatgataggtgttggactatataggaatacccaatagtcaataatgtaaagactaggttggtgagatccttaggttttggaaggctttatgtggcagctctgaagactccaacatctatggcctctagctatgcctccatatgtattttgtaataagaaaaatcatctcccttaaacatgggagagggttcatctccatcgaacatctttctttaggtggtgaagcctaaaataatgagcacgacactttgataccaattgaaaggattactatgcccaagaggtagggtggattagaccaatctaaaatttcttgtagcaataaaatcttatcgaatatcccacttcaccacttgtgcttaagagtgatatatattctatcacaaaaggttttacaccctagtttgaaccctattctagtatgttagttctagtaatgtaaagacaagaattaaattgctaaaatgcaaatgcttaagataaagaagggttagaaacatgacaacatttttccaaagtattaaagagccgggactctccaatagtcctcgttggagcacccacacaaggatattgctccccattgaacttcacaaggatcaagggctctctatgggtttgttcctcgccactctagcatggtgaatcacccacagttgtttacaccttgagtggtgatatacactagatttcttaagtgtgtatcatcagacttatttaggtcaaactatcattcatggcccctttatagtatagtcaaaggaaaaaacaaaggtgctatcactagtatatatgtctatcaactctattaactattagaactagccagtccttaacctttttgctcatccttctaaaatcaataattgaataccatcaataggggcacaaaaactatatagccaattattattactcatagccaataatttagtgttgtcattagtcactaaaactagatgctttcatatctatcatgctcgtgtttgcctgatccaattcctaggtcatgtttgggctccacttagagcttgtaatggcatcacaacatgatctgattaagaaaatgttgactaaagaccttctcacccacatataaaatgccatgacttcgctcaaacttcatcccctagtctcctactacacccatgtctatgcttcgcaacttcacaagtctaccacatgtacttgcaaagggctgattctcctagctcccatgtcgtcgccatctttcccctttaccaccacaaaatcacaaatgtgctcacttattatccccgcgtcttcccttccctaccctaaacactatgggcaacaacgtcgcatcacttggtggtccttgtagatctagacgaggtctcctccccgttttgtgcttgttgatatagtgatcttgtccttgtcttgtcgctgtcctttggagtcgtcgcagagaggcggtgaaaattctagccgcttctagaaggtgcggtcgccggtcggatgagagtcgagtgagtgcaggcctataaaaagtagcaattgggataacattttgaaaaaaatgaaagaaataaaaaaactagggttaaataagattctaaattaaatctaagaaatctatagacattcggaaatctatttatcttttgtactcctttgattggatttctagattttgacgtttgtgtaattgggcttgacagccccatcaatacaggcaggccagctgatttcgggtgccgctgattcggcccatggaggatagagcaggcctttcaacaggcaggcagtccatgacgtagtgcctcggcctcaaagcccatgcgattaatatccactggtacgtacgcgacagccgagggagtcactgagtcagtgccgaacggcctggaaaccccccgccgtgtgccggtgtccacgcgcgccccggtgcggccgccacgaccgcggacacgtgccgggcctcgcacacgcgtcccccacggccccctcgcatcattaaaaccgcggtgcccgcgccacggatccccggcacttcattccggtttccggatagcttacctcccatcgacggtcggtgagccgttggtgagtggcgagacagcgggtgcgctgggcaagaacaggcgacgaagaagatgagcacgccggacacgcggacgctcaaggacgagctgacgagc

SEQ ID NO: 91 - DNA, 636 bp, Zea mays
accctgctccgagtatcgtagtgccccccattgccgcacctgtatcagcgaacttcagcagactcgccttccgcaacctgtacatccgacggacgggtccagacagcagggagatggtgactgtggagggaagaagaggctctagtgatcagacaggcgacatgaggtacgtgagtgacttccccgtctacgatggccgtggctccgacgccgtcctggtggctcgcgtgcagggcgtcacaaccacgttcgggaactccaaccagttcttcaccgtcgccttcgaggccggcagcaggctcaagggctccacgctccttaccgaaggagtggtgacggatggatcagatgaatgggcaatctacggcgggaccggagagtttgctatggcgagaggtgtcgtcaaaagaaggtaccttgccgatagggatggcgccgggaacactgacgagctcagcatgcaggttttctgcccggtgtttggctcatcacagcaaccaaacaagcaggactccagcatctctgtcaccaagattgggttatggggcgccgactttgatttgatctgaacagcaggctacctactagcaattgcctgctcgatcaattatctttcgtaccctttattattaatat

SEQ ID NO: 92 - DNA, 1017 bp, Zea mays
  misc_feature (994)..(994): n is a, c, g, or t
gacacatcgtcatcgatcatcacacgcaatcgacacaagaagttaataaacagcccaaggacgcagagatcatgctgatcgagaaggacttgtactactactcagtattgtcgtcacatgcacatatatgtacataaagagctagctacctgagctctacccaaggtcgcgttgatcgatcgatcatggcgcggttcgtggacccgctggtggtggggcgggtgatcggcgaggtggtggacctgttcgtgcccggctccaaggacatcagcaacggctgcctcctcaagccgtccgccaccgccgcgccgccgctcgtccgcatctccggccgccgcaacgacctctacacgctgatcatgacggaccccgatgcgcctagccccagcaacccgaccatgagagagtacctccactggatagtgattaacataccaggaggaacagatgctactaaaggtgaggaggtggtggagtacatgggcccgcggccgccggtgggcatccaccgctacgtgctggtgctgttcgagcagaagacgcgcgtgcacgcggaggcccccggcgaccgcgccaacttcaagacgcgcgcgttcgcggcggcgcacgagctcggcctccccactgccgtcgtctacttcaacgcgcagaaggagcccgccagccgccgccgctagctagcagctcctctctgaggcatgccagatgcatgcgtgtgcgtgcaggtgcaaccaccgcactgccggcggctacgtatgaccggtgaataaaaagttttactgcaccgtaagcatgctcgccctgttgctattggtatatgttagcagtgtggcagtctgtatgtagtagctattcgcttgcatctatgcactctatgttagtatgcgtacgtgtggttccggaacttttggagtcttatctaaatactagaagtgcccgtgcgtttgtaacgggaaccggacgcgtgggcggacgcgtgggaacgtatgcgacnatagcatacacntcgaatcaacn

SEQ ID NO: 93 - DNA, 10 bp, Zea mays
  misc_feature (1)..(10): n is a, c, g, or t
nnnnnnnnnn

SEQ ID NO: 94 - DNA, 837 bp, Zea mays
ggtcgacttcgctgtcgacgatttcgtacgaaatcgtcgacagcgaaggcagctcgcaccagctagtcagcatcgtcttcgaggccgagaggcttaagggctccacgctgctcaccaacggagtgataacggatgggtcagacgagtgggccatctacggtggcaccggggtgttcgccatggccaccggtgtcatacgaagaaggtttctcgccggtagcaacgacgggaactctgatgagcttaccagcatcgaagttttctgcccggcgtttggctcggcacaacaacagccaaacaagcaggactccgtctctgtcaccaagattggggtatggggtggaggtggagggtcggcgcaggacatcacgacgacggagccaccacagcgtctgcacagcctctccgttcgaactggttttgccgtcgactccatcgagttcacgtatactgatagaggtggccagaggcgcactgctgggcgatggggtggacttggcggcaaccttcgaacgatcgatcttggcgacgccgaggttgtcagggaggtctcaggaacatacggcatgtttgaaggcgccaccacgctgacctcgatcagaattctcaccagcagcagaacctgggggccatgggggatcgaggacgggacacgtttctgcatcaccgcgccgatcggcagcagcatcgtggggttctatggacgctcgaccagcaggctcgtcgctgcgatcggtgtttacctgcgtcaacaactctgatctggtcatctagctatcatcgtcgtccggtgctgtcgtatgtgtgttcatgtcaaataaataaaagtggtcttgt

SEQ ID NO: 95 - DNA, 1125 bp, Zea mays
gctgccactgtgctgtgcagtagttgcaagagctgattggtagctaggtacgctaggcaagtaggcatggctcccaagcggcgcggaaacaaggtggtgggctccgtggtgaagaccaaactagtgcaggagaccgtggaggtcatcgtcgccgacgacgatgggttgcatgcggagaagcagcaggtcccggacccggaggctttggccctggcgcaccccaccgtggacgtctctggctccacggtggtgcatgtcgtcgaggtcactgccaaaagaggccgtggtggtggtggtggccggcgcgaggaggggaagccgccgccggaggaggattctgcagcagtacccgtgccgcagagccaggagacgcaggaccccaacgaggagctggaggacgaggaggagaagcagccggagactccgcgcgtggcgtcggagaagaggaacaaagcagcaacaccaacaaagaagccgaaggcgaagtcgccgcggcggggtggtggcgacgacggcggcggcggcaagaagcgtacggcgcggcggcggcggctggggcaggccagctccggaggcgacgccgggatgggcggcgtgggagggtacaggcggtacgtgtggcgcgtgctgaagcaggtgcacccggacctgggcgtgtcggggcacgccatgcaggtgctggacatgatgatggccgacatgttcgagcgcctcgcggaggaggcggcgcgcctgtccaaggccacgggcagggcgacgctcacctcccgcgaggtgcagagcgccgtcaggctcgtgctccccggggagctgggcaggcacgccatctctgagggcaccaaagccatctccaagtacatgtcctatgccgcctagctaggtagctacatgttttctgtaccttggaagtagtgcgtactgcagctgatgtattttcatgcatgccaactagactagctagctactatacgtatgtatgtagttgatagtgtttatcaggccgggtagcagctagctagaccaacttaattagctaatgtagtttgatgtacctgtctgtcagtctgtgtatgtgtatgtttcgttgttttaatcgaccaacttgttccgtgtcaaaaaaaaaaa

SEQ ID NO: 96 - DNA, 702 bp, Zea mays
gtacgaggcatccgcactactgagagcgacaaagctagctctctccagggatcggattcggatcaccatgccgtccaccgtgaactccgcgtccctcgaccagaaccttgagctcgccaagcaatgctcgcgagaggccgccctcgcagggttcaaggcagcagctgtcgcgaccgtcgcctctgcagttcccaccctggttagcgtcaggatgctgccatgggccaaggccaacatcaaccccgctggccaggccctcatcgtctccacagttgctggcatggcgtacttcatcgcggcggacaagaagatcctctcgctggcgaggcagcactcgtacgagaacgcgccggagcacctcaaggacacctccttccagggcaccggccgtccgcacccagggttcttcaggccttgagcgagagaccatcaaactaccgccctagctagctggacgcactagttcgtcagcggccgccgattcatgtcgtccaagatcatatcaactactgctagcatatatatagtgtactagctagtagtaggcaccggcgatccgtgcatgcatgtgctcggagaaactgtaatggcttagtgctctgctccagtttactgtatctaccttccattatcatataataatatcatcctcctgtttaaaaaaaaaaaaaaaaaaaaaacaagagggggggcccggtaccc

SEQ ID NO: 97 - DNA, 629 bp, Zea mays
  misc_feature (615)..(615): n is a, c, g, or t
gcatacactagtgcttttgctagctacttgtgcgagaagagagagcgcacggcgatagatatggcgcatagcaagcgcgaaatcaagcccgggagggcgcataccaaggtggccggcgacgacgagatgctgcggaccgggttcttcgacggcacgccgctggagggcggcaagatcgccgactcccagcccgtcgacctcttcgccgctgcccgccgcgtcgccgacgccccgtactcatcgcagcaggaaggaccacacgaggaggggaccaacaagaacgccgtcatcgcggagtcggagcccgtcgacctgcccgcgagcgcgcggggcgtggcggaggtgaaccgtgctgggccggaggagcagacaggccgcggtcggcagggcatgaccgagcccaccgccggtggacgtcgcttgggattaggtcgcccggccccggcttaacgatccagatcaagcagtactcgactaccgccactacactacctgctctgctcgtgtcttgtttccagttttctttctctgtaagcggtgtagggtttgccaaacttttatgtacgaattatgaatatcaataaagtcgatctgctgctgcgtttcttggcaccnaaaaaaaaaaaaaa

SEQ ID NO: 98 - DNA, 2040 bp, Zea mays
agcgtcacagagcaccgcccggaggttccccttcccgtcgccgtcgccgtcgcttggccaagatggctgaacaattctataccgtggcatcggatagtgaaaccactggtgaagacaaatcacagccttcattccctgatgttgctgttggcattgacattgggacttccaaatgcagtgttgctatttggaatggccaccaagttgagctcttaaagaatacccgtaatcagaaagggatgaggtcatatgtcatgttcaaagatgatactctttcagcaggagttactggaggtgcagccaaggaaaatgcacacgaggaaagagacatcttgtcaggaagtgccatattcaacatgaaacgcctgataggaagaatggacactgatgaagtggttcaagctagtaagaccctcccatttcttgtgcagacattgggtatcggtgtgagaccattcattgcggccctggtgaacaacatgtggcgatctacaactccagaggaggtccttgccatctttctacttgaattgaaggccttggtggagatgcaccttaagcatcccgttaggaatgctgtgcttacaataccagttgcgttcagtcggtttcaacagaccaggattgagagagcctgtgcaatggctgggctgcatgtgttgaggttgatgccggaaccaactgctgtggcgcttctgtacgctcagcagcagcagcagcttgtgcatgacaatatgggaagcggcatcgagaaaattgctctgatatttaacatgggtgctgggtactgtgatgtggccgtggctgccacagctggaggtgtttctcagataagagcattatcaggatgcacagctggtggggaggatatacttcagaacataatgcgccatgttcttcccaattttgacggtatatatgctggccagacaatggatagaatcaagccgatgagcttactgcggattgctactcaagatgcaattcacaggctcgttagtcaagaatccgtggagatcaatatagatttggggaatggccacatggtgtccaaagttctagaccattcagagtttgaacaagtcaaccgggcaattttcgacaagtgcgaaaagatcatcaaccagtgcttggctgatgcgaagttggtaccagaggacatcaatgatgtcatactggtcggaggttgttcgaagatccccagaatcagaagccttgtcctgggtttgtgcaagaaggaggtctcctacaagaacatagatgctctagaagctgctgtttcaggtgctgcgctggaaggagccatcgcttcgggagtaaatgatccttcggggagcttggatcttctcacgatccaggcaactcccatgaacctgggaatccgtgccgacggtgatagcttcgctgcgatcatccctaggaacacgacggttccagcaaggagggacatgctgttcacaacgacacacgacaagcagaccgaagccctaattgctgtgtatgaaggagaaggggaacgtgctgaagataaccatctgttagggtacttcaagatcactggaattccggcagcccctaagggagcagttgatatcagtgtatgcatggacatcgatgctgccaatgtcctcagggtgttcgcaggggtcgtgaagcctcaaggcccagccattccaccgttcatcgaggtgaggatgccaacgctggacgatggccatggctggtgtgggcaagctctagcaaagatgtatgggaagactctcgaccttgctgttcttccgaagaagctgcagccatgatatgtgccttccgtggcccgtaaagtagctggtgcgttgtgcgaatgtaggctgctcagtgttcagtattcactgtgtcactgtcccctgtggtgtggccactgtttatgtgctgttggtgttgggcagtctacagaactgaactaccagtttcatccttgtaggcgtgtacaataatgtcccgtgcatgtgaaatgaatgttgcggatgtttacttcatagttttcaggaaaaaaaaaaa

SEQ ID NO: 99 - DNA, 811 bp, Zea mays
atctttcgccgtcccatcagcccagcaattcttcgctgttcgaggacccctcggtttcgaccgaagcccagcaagccgaccacacaccgctgccgttggttccgtcccaagagatgggcaagtcctggtcgctcatcagccacctccacaccgtcgccgggccaagcatcaccctgctgtaccctctgtacgcgtcggtgtgcgcgatggagagcccgtccaaggcggacgacgagcagtggctgtcatactggatcatacactccttcgtcaccctcctggagatgctcgccgagccgctcctccactgggtacccgtgtggtacccggccaaggtgctgttcgcggcgtggctggcgctgccgcagtcaagggcgcctccttcgtctacgagaagctcgtcagggaacagctccggaactaccgcgccagatacccgcgcaagggcgccgccgccgccgccgtcggcggcgacgacgacgaccataaggtgcacatagccaaggctgaggctgagcacgaccatgtgcagtgaaagcgtgtggagacgacgcaggagctgaccggagatcatcacaggaacttctgtagatctggtgttccaaggtgttcattcgatgtatcatcctacgtatctctgtaactcttgtggactgccggtgtcgcgtaatgtatctgaactcttcgtgagtgcgatggaacccccctccccttgcagcacgagatgcttctgtaagaacgataacatgtagacaagccaagcgcctcagctccttaagtgaaattctctactactactcaaaaaaaaaaa

SEQ ID NO: 100 - DNA, 1965 bp, Zea mays
cgcctggttccacttccaccacggtgtgcactagcgctacaccgaggccaccttttcttggttccttcaacacgcctactccggcctccggatcgggcaccacgtctgccccctgctcccggaatctttgccaagcggcgccgctactgtccggcaaggcgaccccgtctctcggccggcgttgatctggcgggcggagtggcggcgcgatgggcatctcggcgaggtggctcaagtcgttagttgggttgaggaaggtggggcggcagcagcagcagcggcgaaaggatgatgcagatgttggtcgaatgaaaaccgatgtcgccgaccagtttcattttcagattcagcactctcaagatgacaatagcattgctgcacaagaaattccagaggtttcttatggaaatgatccacctgaagatgattctaatgtaccttcatgcttcgagcctgctcgtagttcagctcatatgccattttgtcaaactgaagaagcccagaaagagatctgggctgctacaattattcagacagcatttagagctttcctggcaaggagagcccgccgagctttaaaagggttggttaggcttcaagcccttgtaagaggtcatatagtaagaaagcgagcagctacgacactccgttgtatgcaagctttggtcagggttcaggcccgtgttagagcaaggcgagttcgcatggctttggaaaaccagactgatcggcaaaatacttcaccagagcacacgatcgaggcacgtgtcagagaaatcgaggatggctggtgtgatagtatcggttctgtgggagatatccaagcaaaacttctaaagaggcaggaagcggcagctaaacgtgagcgagccatggcctatgctctagctcatcagtggcaggcaagttcaagacaacctacagcatttgaacctgacaagaacagctggggctggaattggctagagagatggatggctgttcggccttgggagagtcggttccttggcacttacgggacagatgggatttttgtagttaatgaaaccagacaacctgacagaagtgcaaccaagaccccatacagaaaacctgttaaaaagcatgattcaacccttcaatcaaacatgttgaaccagaaggtcttcccatcaaactcagagggtggtggctcctcgacaaatcgatcaagtggtttggtatcaactaaatctagactgaaggttttacccagagaaggttctgaggaagcctcatctcatccttcagtaattgtgcggtccactagtaatccgaaggagaggatttccagtattaattcaaaggagaggactggggacttggattttcaagttcataaaagatcctccttgcctagcattggtccagaagctggcaaacacctgacaaagaaaggcatggttaaccgatccctgaaggccacaaaagattcccaaatactggggtcaaggcatcatcttgccagttccattgatccagttcccaccagagttgaactggagacttgagaaggcatgttatgctcgaacatcgccatggattgcacctagcagcttggatgccgttcatgcctcaatggctgcatgaatgtgtcgcaacatctatgttatactggtggtggcggttttcgagtttcgacaccatatatgtaggctcatttcatgagataaaatcagatgcatgtgagcatgtccatatatgttaagaggattaccattggttttattgttgtatttgtaagttgccgttaggaggtgtaaatttgtgtcgtcgagtggtacttattatgtgttgtcgtatagttgtgactcgaaggttaaaggtggttttggttctactgcgaagtgtgaactgtagtgagctgtgttctgtaaaaaaataaagttgttgtgatttgtctactgtaaaaaaaatggaagtaacttggttcaaaaaaaaaaaaaa

SEQ ID NO: 101 - DNA, 699 bp, Zea mays
cggcccttgcgatcgccaccacgtcagcgcggaggatgcacggccaccagcagcagccgtacccatacgggctgcggcaagacccgctgcggcggatgcgaggtctggacgtgaaatcggcgctccaggccagcccgcgcacgtccacggtggccacggccgcactcgtcgtcccgctgggtgccgcgctgctgtgcgcgtcggggatcgcgctggctgcgaccgtgaccggtctcgcgctcgccacgcctcccctcgtgattttcagcccggtgctcatacccttcgcgctggccgcggggctcatcgcgtcggggctcctcgcgtcgggcgcgctcggcgtcgcgggcgtctcggcgctcacgtgggccgtcgggtacgtctggcaacggcagggcgaaggcggcggcggggtcactgggatggtggtgcagccgctggaccaggggtcgaagcgccatggtgtccaaggcgccgccgcgttcgtggggcaccgccgcctgccacgggacatcgacgttgctggtgaatgatggtttggcggcgtgtgtgcaaacttttctatgctagtttagtttgcaggtgtgtatggattttgatttatggcctggatggatcacactatgtagcgaacgatgtgttcaatttactgttggaataaaactgcagattggtgttacttaataaaaaaaaaaaaaa

SEQ ID NO: 102 - DNA, 1250 bp, Zea mays
agatcatatgcagcgtggacgcctccgatagccttggctttggacctccttatcaggccttcttcctcggcggcgcaggggacccggcgtcggtcattgccggttttgggccgaagacgcttacccgtgccttcaacgccacctacgacgagttggcgcgtatcctgttgccacgaaccggcggccccatcgtgtactacaccgcggacgcggagccagagagcggtgccgccgaagaggagcgcgggcaggtcgacgggcacgacggtgtgctggacaggggagcgcgacgcgagggcgccggcgcgtgggtgccgggtggcaggggagacggaggcgacgagtgcggcggcagcgatgacgcgcgtgaggcgacgtggtggtggacgaagctggttaacagggtcgtcggaggggcggctggcggcggcggcgctgcggaggcgaacaggaagggcaagaagaagaagggcggcgcgccggagccgtacaacctctacgacagcgagcccgggttccggaacgcgtacggctggaccgtctccgtcgacaagcaccagtacgagcccctcaagcacccggacatcggcgtctacctcgtgaacctcaccgcggggtcgatgctggcgccgcacgtgaaccctcgggcgacggagtacggcgtggtgctgggcggggaaggcacggtccaggtggtgttcccgaacgggtccctggcgatgagcgaggtggtgcgccccggcgacgtgttctggatcccgcgctacttccccttctgccaggtggcggcgcgggccgggcccttcgagttcttcggcttcaccacctcggcgcgccgcaaccggccgcagttcctggtcggcgcctcctcggtgctccgtaccatgctagggccggagatcgccgccgcgttcggcgctcgcgagaaggagttcagtaagctggtgcgtgcgcaacgggaggccctgattatgccgtcctctcctgggaaggaggaggaggagcatgggaagaaagggagggagaaagaggagtcactgccgatggttgtcgagcaggcggcggcggagtgagctgcttgatctttcagtcttggactcttggcttcctcagaagcgcgcgcgcgccaccggaaagtgtttctactcttgttactgcgccttgtcagtgatcgtgttgttggtttttttttttcgtggcgccgtctataaataaaagtgctttgtgcgtttacagcttaaaaaaaaaaa

SEQ ID NO: 103 - DNA, 1856 bp, Zea mays
tcggcgtcactcctatcgtgggaccccgtggtctgcccgtgttcggcagcatcttcgcgctgtcccgcgggctgccgcaccgcgccctcgccgagatggcccgcgccgcagggccccgggccaaggagctcatggcgttctccgtcggtgacacgcccgcggtcgtgtcgtcctgcccggccacggcacgtgaggtgctcgcgcacccgtcattcgccgaccgccctgtgaagcggtcggcccgggagctcatgttcgcgcgtgccatcgggttcgcgcccaacggcgagtactggcgccgcctccgccgcgtcgcgtccacgcacctattctccccgcgccgggtcgcctcgcacgagccgggacgccaaggtgacgcggaggccatgctccgctccatcgccgccgaacagtcggcctctggcgccgtcgccctccgcccgcacctccaggccgccgctctcaacaacatcatgggcagcgtcttcggcacgcggtacgacgtcacatcaggcgccggcgccgcggaggccgagcatctcaagagcatggtgcgcgaggggttcgagctcctcggcgccttcaactggtccgaccacctcccctggctcgcccacctgtacgacccaagcaacgtcacccgccggtgcgccgcgctcgtgccgcgcgtccagaccttcgtccgtggcgtcatcgacgagcaccggcgccgccgccaaaactccgccgccctcaacgacaatgctgacttcgtcgacgtgctcctctccctcgagggtgacgagaagctcggcgacgacgacatggtcgccatcctctgggagatggtcttccgcggtacggacacgacggcgcttctgaccgagtggtgcatggcggagctggtgcgccacccggcggtgcaggcgagggtgcgcgccgaggtcgacgcggctgtcggtgccggaggttgccccaccgacgccgacgtggcgcgcatgccgtacctgcaggcggttgtgaaggagacgctgcgcgcccacccgcctggcccgctgctgagctgggctcgcctcgccaccgccgacgtgccactctgcaacggcatggtggtcccggctggcaccacggcgatggtgaatatgtgggccataacccacgatgccgccgtgtgggccgacccggacgcgttcgcgccggagcggttcctgccctccgagggcggcgccgacgtggacgtccgcggcgtcgacctccgcctggccccgttcggcgccgggcgtcgcgtctgccccggcaagaacctgggcctcaccaccgtgggcctctgggttgcccgcctcgtgcacgccttccagtgggccctgcctgacggcgcggcggccgtttgcctcgacgaggtcctcaagctctccctggagatgaagacgccgctcgtcgccgcagccatcccccgcaccgcctgatccgtcctgccgccgacgcgtcacgtcacgcgttgtttgcatggatgatggtatctttgtctgtctgtgtggtcttcgctaaagtttgcttcttctcgatcgtcggttcgttcgtgcctccaccttagcctagggtttggtttcttgcaaggtagtgagtgtgtcttagtctcaccatcaccggggctccaattttggaaagctgcgtgttaggagttaacccctagacatgtttgcgtcttgatcgccaccacccatcagtatcagcgcagaaactacatatagatcagtgtttgtcgaccagtcatggaagtcgtgtgctctcaagtctgatgtattatatacatatatatgtattgtaatgtgattatcaagaaccgtgctatttacatattgctgtttaattgaact

SEQ ID NO: 104 - DNA, 1765 bp, Zea mays
ttttttttttttttttatgcacaagctgcagactttttattcctgaaagcacagcttatctttaagcagtacagactacagacagaccactgactgcataagtgaggacaaacgggcacagctcaacgaggaagcaagaacacagaacaacaaacaacaactccagagctcagaagttgtcttgattgcccatgggcacacgcacaggcctaaccgtgccatgcatcgacaccatcagcgacggagatcgttcatgacctgctgagagccccggaactcgttcatgaacgccttatggaacgccctcaagcgtgcgaccatcgcctcgatttttctttcgtcaggaaggatcgtgcatcggatatggatcgtcccagaggcctggtggaacccagagcccggcacaacggcaatcccggtggcatcaagcaggcggtgagcgtagaatatgtcaggtgaaactccctcgaaatcggcggccctgatcgcagcggaggggaggtggagccgtgggaacaagtacagcgcaccctctaacttgttgcaagtcacgccctccaggccgctgaaagctttctccagggtcttggctcgcttgcagaaggatgaacggatctgttccttctccttatcaaatgactcgaaagattcatctcctagcttcggtggatccattgcaaggctgacgagaatctggccagcaatgttggggcagagtgttaaagaagccaccttgtaaatctgatccttcacattagctgcaacaccagacacctccatgtagcctcctcttctgccactctccccatagctcattgaaacggaatgaagtgatactagggtgaggtcattttcgtcgtaagcaagtgatctggccactttcttgaaggaatggaacttcttgttctctgcgtagatattttcctggtatacctgtccagtagggttgccgggatttattaccaccatagctctaacagtgaggccagccgaacgagcctcttccaagcacctcttgacttcaaagatctctaggccccaaccgttatcttcagtgagattgtacggcaccatcgttccaccgtgcagtatgatggaggccgagtacaaggggtattcgggtagagggctcaagatgccgtcttcgtgagacctgatgagtatctgcatcatcaagttgatcgctgaggtggctccatctgtcagaaagatatcgtccgctgtcgatggaaaaccgtctcttgcggtaatcccatctgctactacttgacgcaggcatttgattccccggcaatcagtatatccaccagtgtctttgctggggatcgagtttaggatcctcctggcccttcttatgctacaagggctgaataaggcacgagcttcatctctgtccatgagggctgggttgtcgcacagggagagaacctcacgaaagaaagttagtggcggctgaccaaggacctgggggttcccgaggttgcagtatattatctcttggaaagggatagaagcagggttctcctctatctcctgctccagccgcgctgcgtgcttgacgatctccccgcaaggcgcgtatctgatgatcttgaccttcaaacgaagggacaacgtacgtcacctaagcttttcagtttaaatcggaagaatgcaatggatgaacatatatatgatttctgaagaagaaaccctgggatttatggtctcggcggtgatggacggggccttgttgtacgacatggcgcgtcggggcaccggccgg

SEQ ID NO: 105 - DNA, 1047 bp, Zea mays
ctcctcctcgatccatcgatcactgcaccggccggcggcaccgcgctcgcaggggctagccaacgagacggcagcaatggcggaccgtgaccgcagcggcatctacggcggcgcccacgccacctacgggcagcagcagcagcagggaggaggcgggcgcccgatgggtgagcaggtgaagggcatgctccacgacaaggggccgacggcgtcgcaggcgctgacggtggcgacgctgttcccgctgggcgggctgctgctggtgctgtcggggctggcgctgacggcctccgtggtggggctggccgtggccacgccggtgttcctgatcttcagccccgtgctggtccccgccgcgctgctcatcgggacggccgtcatggggttcctcacgtcgggcgcgctggggctcgggggcctgtcctcgctcacgtgcctcgccaacacggcgcggcaggcgttccagcgcaccccggactacgtggaggaggcgcaccgcaggatggcggaggccgcggcgcacgcgggccacaagaccgcgcaggcaggccaggccatccagggcagggcgcaggaggccggcgccgggggaggtgcaggtgccggcgctggcggcggcggcagggcttcctcgtaagcaagtcatccatgcatggattatggatagatgcgcgcgtgcgtgtctatcagtatcagcagccagcagggtcgtcgcggaatgctgtgttcctgtacgtgtgggtgaccgtccttccgtccttcgtctttctccccccgagtgtgtgttacgtatgtcctggtgttcgtcgtgtgtgttcatcgccgctccagttgaattccggtgtctgttcatcgccgctccaggtcgtagatgtgaatatactttgctaggggaataagtgataagtctgtctggaaggtaatgtttgagctttgctagtgtggctgggcactctggtcactggttgtgttgtgcatgcatcagctgtatgatcgtcgtctgttgtggaaaattggtcaatgtattctcttgctgaaaaaaaaaaa

SEQ ID NO: 106 - DNA, 540 bp, Zea mays
cgatgggtgagcaggtgaagggcatgatccacgacaaggggccgacggcgtcccaggcgctgacggtggcgacgctgttccctctgggcgggctgctcctggtgctgtcgggcctggcgctggcggcctccacggtggggctggccctggccacgcccgtgttcctgctcttcagccccgtgctcgtccccgccgcgctgctcatcggcacggccgtcgcggggttcctcacgtcgggcgcgctggggctcggcggcctgtcctcgctcacgtgcctcgccaacacggcgcggcagggttccagcgcaccccggactacgtcgaggaggcgcgccgcaggatggcggaggccgcggcgcacgcgggccacaagaccgcgcaggccgggcacggcatccagagcaaggcgcaggaggccggtgctggcactggcgccggcggcggcaggacttcctcgtaaataagcaaatcatccatggatgcgtgcgtgtcaatgtcagcagggtcgtcgtggaatgctcttctctgtacgtgggtaac

SEQ ID NO: 107 - DNA, 877 bp, Zea mays
tcgccaccgccgaccggactcccctcaaaggcggccacagcacagcgagcatgggagaccggcatcagcatgggcctggcagcggcgtggaggacccggcgacgctgctgcggcgggtgcagacccgcgcgcccaactcgacgcaggtggtggggttcctgacgctgctcgtctctggcgccgtgctgctgctgctgacgggggtgacgctgacgggcgccgtggtggcgctcgtcttcctgggccccatcgcgctgctcaccagccccatctgggtgcccgtcgccgtcgtgatctccgccatcgtcgccgccgcgctctccgcctgcgccttcgccgccgccgcgctccccgtggccacctggatgtaccgctacttcacgggccgccaccccgtgggcgccgaccgggtggactacgcgcgcagccggatcgccgacaccgccagccacgtcaaggactacgcgcgcgagtacggcggatacctgcgcacccgcgccaaggacgccgcgcccggcgcgtaggcgcgcgccgcccggcgtgcatgtgctgcaacccccgtccgtggtgccgctagctataggtcatcattgctgtcgtcgtcgtcgtcttagttcctcttgcattgcgtgtgcgtactctgctccaatcatcccccgcccgtgttgtcaagcaatttctggtcctcttgcttgctttctctaggagatgctgttcatttcgatctcttggcgcaatttgatctttcgcttggtgtaattaggtcattagttaaatcataagagactcgtgttcatggtctttctcacaaccttaaaacaactaattgttgttgaaaagaatatatataggtaaccttgctcaaaaaaacaaaaaa

SEQ ID NO: 108 - DNA, 846 bp, Zea mays
atagcttacctcccatcgacggtcggtgagccgttggtgagtggcgagacagcgggtgcgctgggcaagaacaggcgacgaagaagatgagcacgctggacacgcggacgctcaaggacgagctgacgagcatggacaggaagtgcctcgtggacctcggccacccgctgctcaaccgcgtcgccgacagcttcatccgcgccgctggggtcggggcggccagggccgtctccagggaggcctacgtcgtcaccgtcgaagggctttcaggagactcgtccgggctagacgccgacggcggcaagcggagccatttctccagcatcagaggtgacgacggccagaggtcgctcgacgcagtggtaaaaacggccggcaaggaggccttccagtgggggttggcagccggagtctactcagggctcacgtacgcgctgcgggaggccaggggatgccacgactggaagaacagcgccatcgcaggcgccatcgctggggcggcggtggcgctcacgggtgacgccggcggccactccgacaagctcgtcaacttcgccatcaccggcgccgcgctctccagcgccgggagcttgctctccggcatattctgattggcccccagggctgcacccgcctgacacgggttcgctgcccggttgagcccttcatccgaggggttttagcatgaaaggttgatttcaggccagcaatgaaatatgtattcctagggccctgccggtctagcactgtgcgcttccagtgtaatcgttgcttcgtgtacgagtttcagacatttcgaaatatcttctccgagttccggtggtttgacaaaaaaaaaaaaaaaaa

SEQ ID NO: 109 - DNA, 1106 bp, Zea
ttggttttttgataatttgtttatctcacbvhbvhgttctaacaatbvhcgcagatcttatacatctctctcgtagtttttcataaactacgagaaagatataggttttbvhaacaaatttacttttattttgtcatatabvhaabvhgtcaaaatataaattgtacatcbvhbvhagttatacaaatttgtagtccaaaatcttttcatttaaattaatttactgctttaaabvhtgatttttaattgtctttgcctagtgttgaaaaaaagcactcggcaaagaaactctttgccgagtgttaaaaaaacactcggcaaagaatctctttgccgagtgtcaaaaataaaatatttggcaaatagcttctttgctgagtgtttttttgcttagcacttggtaaagaacttctttgccgagtgtccgaaaaagcactcgataaaatatttggcactcagcaaagaaccgaattccagtagcgtgagtcagaagttggcatttgaaccctttgaaataatcaaacaaatcaacgcagttgcatbvhaattggctttgccacbvhgtagtgcagtccatcgagtgttccbvhtttcacagcttaaactaacccbvhgttttggtaacagaacacacaccagtgactccatcgttaccggattttgatactacactgaaggttccggaacaaatactggattctagattgattctagattgcttcgatcaabvhgggatcatcaacctaacacagcttaaattatacbvhcbvhcgtacatttggtccttcaagaggaggcaggtgbvhatcaaggtctctctagcggcctggtgtgcagccacgtacccatccacgcgtcgccgcgggcgacgacaggtgttggccaacctggacatcagacaagcacgacabvhacaggcgttcgccaagcgtacgtacaggagacgaacacgacaagtagcaccgtcctggccgagagcccgtcbvhctcbvhcbvhcbvhcbvhctgatcbvhcatactcgataaatatacagcagagagctgagctacgtacacaaacaagaagcttgcattgttcgttgctcgatcgttgcaggtabvhgaga

SEQ ID NO: 110 - DNA, 922 bp, Zea
ttagtagagaataacacacatctccctaagtatcacaaaaaaattaattttaagaggcaattttagtttccaaagcgatttattaagatttaagggagggtatttattagaaggaagtacattgatcaaacagagaaaattccatcattbvhgtcccccatagaaatttgtcactcgattgtcgcaaaaaaagttaccagtaaatttgataaattttggataccggtctgttttaactcatcaagaatagcacgacgaaataatagattgcgtacgttttcattcataacatctctcactaaagcattttttacacacgctattaaabvhgttcggaaactgatccttaagcggtctattcatatccatctatcgttctaagattcttcctgtacgtagattctttccgattaaattctcbvhaaaaattaaaaacaaacaggtccttaaactagtcgccggaagttctcgcttttgctgtagttgtagtgtcgbvhcbvhggtggaaacgtggacagatagattcbvhaabvhtggacaacccacctggtccagagcatcbvhatacattgatacacgtggcacgctgcccactgbvhccctcagctttgaagctaacgtgtcaagtgtggccggcccctgcattgcgtcgtgccaatcgtccaagtcccbvhgcaaaaacccagcgctttgtgccgccgccgtccgccggcccctctgcccttgtacgtgcacctagacacatcgtcatcgatcatcacacgcaatcgacacaagaagttaataaacagcccaaggacgcagagatcagctgatcgagaaggacttgtactactactcagtattgtcgtcacbvhcacatatbvhtacataaagagctagctacctgagctctacccaaggtcgcgttgatcgatcgatc

SEQ ID NO: 111 - DNA, 1945 bp, Zea
gattcagaacatctggtcagcttaabvhbvhtggtaaagggagcattattcttttattttgttctttgttctgtgcabvhgccatatattttcgtattgatbvhctctcagtggaacagtctgaagctabvhtgaaacttcgbvhttcaggtbvhatabvhtaccaacagagattaaccggctgagbvhcagagttaactatcbvhcattaaagttcctacctgatattgaggaabvhgctgttaagcttgctgcaagabvhaggaacaaaactggcagcataaatccatacbvhtacgttataattatccttttcgctgggtgatattgtgtacagtagagcttgtatctacccaagacactatccagtgcatagctgaagttgacgctctcaaagtttatttcacaatcaaatattgacacattttttttgttcttaaagggctcttcacctgagatttgaaaaagggbvhgtggggctttctttctgtgattttgccggcacacgggaggagaaggcabvhbvhgcggcttataggcagaaagabvhgccaaggcgcttcaaggtgacgcggactggactagatacatctgttacattggactgagaaagaatccctaggagaabvhaagcaccgacactaactatacgcgctagttcgggaaccccgttttctcacgagacttttatttttccaagaaaaattagttcattttttttaggaaaatataaatctcttaagaaabvhtagttccgaaactagccctacaagtctaacgattttctcttttttgtatctgccacgcagabvhgatcccatctbvhgccactggcgctgcagaagagaaaagaagggcggtgccctcttgagcccggtgagatcgctgtgatcctgcgggcactggggtacacgagcggcacacagatatacgtcgcgtccgggcaagtgtacggcggcaagaaccggbvhgctcccctcaggaacbvhttccccaacctggtaggcaggctcaaatccacttcagattgtgtcaagctgaagccagtgataactgataagccactgccgtgcattgtgcatcgctgcaggtgacgaaggaggagctagcgagcgcggaggagctggcgccgttccggcggcacgtgacgagcctggcggcgctggacttcctggtgtgcctgcggtcggacgcgttcgtgbvhacgcacggcggcaacttcgccaaactcatcatcggggcgcgccgctacgcggggcaccgcctcaagtcggtgaagcccgacaagggcctcbvhtccaagtccctgggcgaccccgacbvhggctgggcctccttcacggaggacgtcgtcgtcacgcaccgcacgaggacgggcctccccgagcccaccttccccggctacgatctctgggagaacccgctgacaccctgcbvhtgcagggcbvhagacgggccatccctcagatcagagtggccgtctggccgagtcctcgtcttcgctaggctacgactacgagtagtctcgtgcccacttccatcagcgcgcatttcagcgagcatbvhcttctactagttaaacacctagatttgtbvhtatttggctggcctaatctgtagtttcacatagatcacttcbvhctgaaabvhtcttggacaaggcagtgabvhaagataacatcagtaacgacctgcttttccaaagctcctcttggatttaccaagctgagccaagaabvhgctaacctgagttttgacttgcagccttgcaggcbvhacgtgggcgacctgaactcgctagtcgtagagtttctttccacgcggcagctcggcagagctacgcagcctctctgcccgccacgttctcctgcaaggctgcaactcgaagcactgagaagctattaattabvhgattctcgagcbvhcggccttgttcgctaaacct

SEQ ID NO: 112 - DNA, 512 bp, Zea
gccagtgctabvhatatttacactagcgggctgctaaagaaaaccgccagtgctaaagatatttacactagcggttggtgaacaactgcctgtgaaaaaagccgatttctactagcccctagctagcactggcgacataaaaaacgtcagtgaaaatagctctaggatcgtcactatagagcttctbvhtacttagtggttagaactgatattgtagtgcaccaagtgccgattttaattaaaccaatactaaatactagtaaataatactagtggtctgaattcgatttctatagtabvhtttgcttgcaagccgcaaatagagtaaacattcgtcgtcacagaaatccacattacatcaaggtccbvhgcggccggccacgtacccatcccacgcgtcgctgcggaggacacgtgttggctgaccggacagttggccgatcagacagtggacagaccggacaatagaagaagaagacgacgacggcggcggcaccgccgagtaggtgcat

SEQ ID NO: 113 - DNA, 698 bp, Zea
gtgattaagttgactggcaaattgcatagacgataatacactctbvhcttttgagataaccactgtacaccaaccgacttgttgtttgaaggctgatctttaaccattactactgtaactaactgttgttgaataaactattcatacgacttggagactttgbvhtatattbvhttttgtacctatactattaactaccgcagatacgtatttacatctcggtaacatctgccagtcgcacaabvhcactaaggtgactgacagactgtagcgaccgatagagcgctgccttgttctagtcaaaattcaagcctggtggtcttctcgatccctctgcgcgcgccctgctcttttatcctgtgtgtctcctccacgagggaaggcgtcbvhagaggacgacacgacgaccbvhcagtgctgcgcaaccgcgcgcgtctgcctgttcactgagccccaggcggccggtccaagctattaccacgtcgcccccctccagggctgcgggcagcaacgtgtcgaccggtccgcctctgacgtgtccctcccgtcctctgcataaaagggtgcggctggcggggagcctcagcttcatatcgtcgcaagctctccgtgtttctctcgtttcttcactgctgccaactgtgctgtgcagtagttgcaagagctgattggtagctaggtacgctaggcaagtaggc

SEQ ID NO: 114 - DNA, 1355 bp, Zea
tataaattagaacggagggtbvhtttttttacgtttccattatttgagtctgctcatcacgtttaatttgtttatacagttcabvhagttcagttacagcggbvhataattcgacggcgtttatttgcbvhcacttactttttacattacgcctagabvhtaatcggbvhgacgaatattacttgttttgtgtttattttcatatttttttctcttcggatacggatattatcgagttgtgccgaacaagattttgabvhtatatcgacatcttaaatatataactttgaatatacggatacgtatatagatcggatattgaatacccagctagactcagattaaattggatcttagttattttagggggtgtttgabvhcactaaaactaatagttagtggctaaaattagttgaaacatccaaacaccctatctaatagttcagctattagttatttttggaaaattagttaatagttaggtagttatctgttagctagctaattcaactaattataagttctagtgcatttaaataccccttagacggatcgaattagaacaccgbvhgataatatccataacattttacacctataaatbvhtcctatbvhtatatagbvhtcaaaaacaggttgggcbvhttggbvhacccgtggcacgatacagttttaagcagtgccaaacbvhgcbvhgcggcgagctagacbvhatacggtccggttttatttttgtatttttttttcgtaataatatctgtattbvhcttgttcgaacctcacttgcttbvhtggbvhtgatagtatcaactcgctggcgttccgtgcatcgtaacatctatcacatcccacagatcbvhtcgcttatcttttctgatactatagcagtagcacacagbvhccggtcgaacccagaagcatcacgccatcaccccctccccttttcccaatcaaaaccacgtccgtcccabvhgtaacccagcaccaatctcgtgttcgtgtgagacagagcbvhcaaacaaacagcbvhcbvhcgcgaaggaaagcgtgcagcgcgcgggacgtgaaccgcgcgcgaaccctgccgcccgtccggtctcagctcacgcccagcgtccttcccaggccgcgatccggccatcctgacgcgcgtttcgtctgggccccaaaaccacgtccgccgcggcggcggcgcgcccacgggctcccgtgcccgccgatagtttgccggaccctgccgcctcctataabvhcagagccctccgccagcgtctcccgcatctcatcttcttcctccatccgcactactgagagcgacaaagctagctctctccagggatcggattcggatcacc

SEQ ID NO: 115 - DNA, 1941 bp, Zea
aagggactcgtggcctacacacgcgcacaataatcagttaccbvhaattattttatbvhcgaatatcgacattacacbvhaatabvhtgaatttgaatttccgcagcbvhcaaaattcccgtgagtttgaagggcgataaaaacgggtggccaaaattggggcggcabvhcaagaagataagcctatccatcccttgcattggtattgtcatctggggcagggcaggggagggcaggagaagctatctattggccatctatccagcagccgctctccccagccccactctctccatctagagctccctaactgcaccggccgccacaccaccgtaccccgacccctcgacaacabvhgccaccgtcctaggcagcccccgcgcgccggccttcttcttctcgccgtcctccctccgtgccgcgccggcgcccaccgccgtggcgctgcctgcggccaaggtgggcatcbvhggccgtagcgccagcagcaggggcaggctgcgcgcgcaggccacctacaacgtgaagctgatcacgccggagggggaggtggagctgcaggtgcccgacgacgtgtacatcctggaccaggccgaggaggacggcatcgacctgccctactcctgccgcgcggggtcctgctcctcctgcgccggcaaggtcgtctccggctccgtggaccagtccgaccagagctacctcgacgacggccagatcgccgccggctgggtgctcacctgccacgcctaccccacctctgacgtcgtcatcgagacgcacaaggaggaggagctcaccggcgcataatcattcbvhcccatccatctctatctctgtgggtcgtcgttgtgtgtaatttgagtacgcgcgtacacgtactgcttttgtttcatttgagcactgttcgtgtaagcgtcgagttgccctgccatcgtctctctctctctcgatctctactatttctgtgtgtgcgcgcttctatttttccabvhttgagttacgcatatttgtccagtaaccttgttttgcttaatcacacacagctcccaaattccagtcgttctgtggtgacggttggttgtggggtgtggcggcgacgtcagtcbvhggagaagagatccgabvhcbvhggggtgttggcgctttctctgtccctctccctctccctgtcttctcgcgcgctcgccgtcgtcbvhgctcgacgaacagacattcactgccttccccgcgcgcttcagacagaccacgcttgtacgtagcgccgcttctctagtcgccggagcaggcacgagtgggtggcgaagaggccacggcccgacctgctagcgccagcgaccgcatctcgccaccagccgctgcccgctggccgctgccaccgtbvhttgggttgggctgaagcctgaagcccgtgacctttttctttttctttattctttccttttcttttgccggacaagacaagtgbvhttgcaaaatccatataaaatcctagagaaactataaagtttttttttgttgaactccttataacaacgtctaccctttaatatbvhgtatataatttcacbvhttttagtataaattgtttactgtatttctbvhtctatbvhctcttggtaagattgtttataaattttaaatttaaaatcttaaaagtcataaacttatagaaaaaaattaggatcctaaacaattttaaacaaaaaaaaaaacttttcaattacaaaatcgtbvhtgttctttbvhcbvhcbvhcaactgagaagctgcccaacggccagcgcagcagaagggcagagcggtgggcggccacgtcgccgccgcaaaggaactgacgtgtcccggcccgacgacgacgacgcgaaccttcaccgcgccgcgctccacctctcttaagctaaatacgctagtgcaggcgcaggcatccagctgcgacaacgta

SEQ ID NO: 116 - DNA, 1188 bp, Zea
gagcgacctcggactcagcggctccgcctgtcggcgagcgcacgccgcgtgcctgcccgcggtctctcgtcgtctccagaagbvhcgccccgatactcgtctttccacccttcttctacctccttccacccccttctccccacgccccatcggatcgcacgcgcaccgcaattgttgttcatcacctctccgaagaagcgtcacagagcaccgcccggaggttccccttcccgtcgccgtcgcttggccaggtaagtabvhactaatcgcctgagtctcctgcgcctcattacttcgaactgtggtttcgttagtcggacgtcggagcabvhctbvhaaccttgccattccbvhttacttggtgctgcgtgtccacattcgacgccacatttttttcttgctagccaaccattgcgcgtctgtgttacagttccaccggcgatatacattbvhcgatcatttgbvhtggatattggtgtttttttggttagcggatcaccttagttttgcactgaacbvhgaacaabvhtggbvhttggtgtttttttgttagtggatcaccttagttttgcactgaagbvhgaacgaggaabvhgggaaggggaactgaacgaagcacatagtttcagagactbvhtgcactgcagagactbvhcgabvhgtcaagggaatttctcgccttggtttcctaatcctcgcttccttcttgtttcgaattcggtgtatatbvhtggtttattcggcacgtttaggaatttagttggggabvhgggcgcagtbvhtcagggttgtgtcatctgaatatatabvhcaagatctattactagtttaaattgacttattbvhcaagtcgattaacctggctgggctcagtggtgtaccbvhgaagctttgtgattgtagccgggcagtagaacgctaaacctgaggtttggtactgtagcactcggctagtcagctgtbvhccaagbvhttacttactcgatactttctgccbvhtctcatctgtgttaaccagggtgtgtgccacatagggcttctttccbvhctgtttgttaacctgttcacacttcttccatatatatattcataatagctcgatctacaattttcctgtcttgagcttctttgttcctaaaaacgaattgtataccagcabvhtatatctgbvhcaatattgtttgtagg

SEQ ID NO: 117 - DNA, 1507 bp, Zea
ctacatttbvhttatagaggcgcaagtagaagabvhtgttataagttgtacatcggaaaaatagcbvhtaaatctatagaatcaattttcatctttcaccccbvhaatttgaggtbvhcttatbvhataactttagaaagtggtggabvhtcatattctaaaaaaaatagcctatttcattagtaagattccaattcctcgaabvhaaagaaaacaaacggggccttattagabvhgaattcaattccabvhatccaabvhgggcgtaagggatttccactttcctaagaaaaattagctcattttcccttggtgtttaataattbvhttttgttaaaaggtcagcttcccgagabvhcgttgtgtaaaagcccagtataagggtctgtttggttgggctgtggctgtgaaaaaagttgctgtgggctgtgagctgtggaaaaagctgctgtagactgtaagctgttaaaaagctaaaaaccgtttggtggaaaccactaaaagtcgttaaaaaatcttcgatatbvhttttcacagttccatccgaaaagccactaaaagcaggtccagaggtgctttcagatttgcactacgagaaagtcggtttttagaaaaagctgcttcctggatccagccctttggktggcttttggctttwaggggcacaaaagccaaagccaaaagtcaaaccaaacacaccctaagtcgcgtgagtgggccatttatctattccctbvhcaaatctcctgaaaaattacaacattagctcgttaaaaagtaaaataattttaaaaaattagagactaattatttgataactttatagttcatcacattttcattgaaabvhttttattttttttgccgaactgatctacacaatatttgaatctacacttttggcttgggctcggagaaaaatagtgcgtgcccagccctttgctctttccccgataacaacgcggtbvhtggcctaacgcccaaatccggcccatttattccggtgcgtagbvhcagacgcgggagcaggtgccaagtcactggctcactgctgcaggtggagtggtggacactagtgccgcggttcatctgacacgtgtcgccacgtgccgccbvhgcagcacctcagcccggccggcgggccgactgacgtcttgggcaaagcggcgagcgacgcaggcggcgaaagccatccgatttgacccctcgctagacctttcaagaacgaacgctgtgctgctcagatcagaccgtgtctgcctcaaagcgbvhccaggacgccacgtccaagcaaagcacccgbvhccattgccacctcccagcactcacgcgtgagcgtgactataaaaaacgcaccctctgcatccgcccccgtctgcctgccctaccgaatctttcgccgtcccatcagcccagcaattcttcgctgttcgaggacccctcggtttcgaccgaagcccagcaagccgaccacacaccgctgccgttggttccgtcccaagagbvh

SEQ ID NO: 118 - DNA, 910 bp, Zea
catacgatttcctaagcggaatctcgagacgaaccacatttttcatccggtctbvhagcgcgctcaatcgtcagtgacagagaacactgtaggtccttaactggaactaacaattcagaaacaatcctaabvhtttattacactggbvhgattttatttggatagbvhatactaccgttcgtgggctaaaaagcaaggtaaaaactggcctaacgcacgtgatcctcccbvhgtctcagaagaaaaccactcgcaaaaaaaaaaacgcagcabvhaggctgctttcagttgacggtgtgcacgactcgctgaagccagcgtgattttgtcccggttcttggagcccagcaaaaagggcgcctgaaattccacaggttgaaggggaaaacggcaacaaaaggggctccgacbvhcggtgcagaaagtgaaaacgcgagaaaagcgcccgtcctttctcgttcccttccgtgcagttgcaagtgttgccacgccgcgccgcgacacaccacgcaggaggcagcgagcgcaatabvhcgcacgcagaggagcacagcggcgtcactgctgcggcagtabvhgccgcctggttccacttccaccacggtgtgcactagcgctacaccgaggccaccttttcttggttccttcaacacgcctactccggcctccggatcgggcaccacgtctgccccctgctcccggaatctttgccaagcggcgccgctactgtccggcaaggcgaccccgtctctcggccggcgttgatctggtgbvhcgcttttcacggtgtttttttttttttttgtttcactcccccctccgtcctgtgtgattgtgctgcagttttgtgccggtgctaacggaaaccggaaagtttgcgtctttbvhttactgtggttgaggcgggcgg

SEQ ID NO: 119 - DNA, 1131 bp, Zea
ccagccatcgtgcttgagtgabvhtttttttgcgtgtgtgtaggtgtgcgtgtgtgtttaattgatataaaataacccaaccaaacatctcctaagatttccataagacagagaggtgaggaatagtattagttctcaabvhaattcaatcaactaaatatcaccatcatatcaacattaataatagaacaataaaattttcbvhatagagaggctggtaaactttattttttcatttgattccagtgtggaaaatagtaatataagcagtaaattttttcttaaaaaagagtggaatattcacctttttatataaattagttattcttcgaagcgtcacacacggbvhtcctaaagatattcttcaactgcaacacaagcattctgaaaaggttagtaacaatcagattabvhttgtaaaaatbvhtgacacgaaaatttaaagaacgcgaagtacaatacattggtaagaaatcaaagacacatattaaccaactattagagaacatatacbvhtttcttctaatcctggttaggacataggacgagggtttcacttctatcacabvhtgctgcaactttcagagttaatcaaatctgttattcagtgttbvhcagcgacaaattccaabvhtataatcccacgbvhattcbvhtgactccatttcgtctgtaattgbvhcgaaactgccgaattgttactgctcccataagttaattgtaccacgcttgbvhaagacgttcacgtatcttgtgttcgatttbvhtagccgttattcagaccattgcagcgctgagcttcaaggcaattagttttgccccgcgbvhtgaggcaggtgbvhacacgcgcccttcgggccgbvhccgacgcgccaccgtgcagactctaccgaggbvhagagcagcgatcgagagacgtggagcgaggaggagactttaggagaggagagaccagagccttaagcggtgacacacccabvhatcaggaccacgcttgcacgtgtcaggtgcctctcctccacgcgtcgcgcgcatcacactgatactgtcatccagctcgacgctatataaagtggtaagcatcttcgtcctcggggccgccccaacggcccttgcgatcgccaccacgtc

SEQ ID NO: 120 - DNA, 563 bp, Zea
cattgttatacatcggtgbvhcagatcttcgcttcgcbvhggtggcaatttagtaacagaattgctctbvhagttattgcttttagtttctgtctttccccaactgcgtttgagtttgcattgtcacbvhtggccttttttggttgtaagacttgtttgatcggttgtggtgggctgtcatcctttggbvhtaggccggaacctcttttcccattatctaaaaaaatacatcgtcatttccagtgagacccagcggtcgtgtcatcagaaaagcggacacgcgtcgcgtcgcgaggccaggtgggttbvhcacggacctgctbvhctagccbvhctccaggctccagctccacccgtccaccgacttttcttctcaaccaggctgcctcctcaccbvhcacgacgacacgtgcgbvhccatcacgcatccacactcacctgttgcctctatatccaccgccccaccgccgtagcaccagaaagaabvhtacagtctacacagctgaccagagagctagtgcaagcgatctagcaagttttaatcttggaagaagccagctagg

SEQ ID NO: 121 - DNA, 991 bp, Zea
atcatcaccctaccccgagctgactcgggccgcagggaaccagaccggtgtcccatctggctagctccgccagataggcabvhaaggcgccccgcbvhctctgtgacgacggcggctctcagcccccttacggaagcaagaggacgtcagcaaggacccaaccgctccgacagctgtccctccgccaggctccatcgctcctccgacggccacgacatcgcaccagctgggtgccaaaatctctccggctgccacgacggcbvhtacttagggcgctagctctcccccgctagacacgtagcactctgctacaccccccattgtacacctggatcctctccttacacctataaaaggaaggaccagggccctctcagagaaggttggccgcgcggggacgaggacgagacaggcgctcgcttggggccgctcgctccctctcccacgtggacgcttgtaaccccctactgcaagcgcacccgacctgggcgcgggacgaacacgaaggccgcgggattcccacctctctcacgccggtctccggccgcctcgctctccccccttcgcgctcgccctcgcgctcgatccatctgggctggggcacgcggcgacactcactcgtcggcccaagggacccccggtctcgaaacgccgacaatbvhatatatagaaactatabvhatacacbvhcaattaactagtgctgataatatatttacactgtagatttttccgataagtagaaaccaaatatttcaccgccacaaaaggcttcatcccatcagttacccbvhtagcaaaaccaaaatacttgtgtgcgagaggccaagagcccaaaccaccgccaaggaaacccccagcccaccaggcgccttcctctataaataccctaactccggtggccgaagttcctcacccattcactcactcatctcaaggcctaggtagcaccgtagcagctactagaagcagctagccagaacaactcgtc

SEQ ID NO: 122 - DNA, 2519 bp, Zea
cttccgataaaaatatttggaaccgcatcctgcatatttatttagaaacattgcatcgataaccgttgcagagcaatatacagggtactcaccaactaaatccataccatacagtgacaggtgaaattggaatacttgatcagcttgaataacagagagcaaagataaatctagccctggtaataaccbvhgctcacatcttbvhtacaagagccaattctaaagacacagctgcgggbvhtagcagttaggcaaggaaagaaacaaaattaccagctaactgctaagtgtcaaaacattgctttacactaaagggttaaatatttgccaagaaggtagtgggcacctctcctctgcaagcagcagcataatctagggcgaggcttggtgcagcttccttgtaggggcatbvhaacttatccbvhaaggcactctcactcatctggacagcagtatagtagctccggtcctgctccaacaatccattttccttcacgcctagtttggatcactgtaattgaattccattctaataatagtaatttagacatatatcaattaagataattcacttttgtacaaaatatatttgtatactattattagcaatatatcctaaatatttbvhtgctacatttttactatagaggagtagaacgaagagtgtcatataagttatagattagaaacaaattctaatcbvhcataaaatcatttcccatcctccaccctbvhaatttgagataggcttatatctgaactttagaaagtggtggabvhtcaaattccaaactaaataagttactttattgagtgaattctaattcttttgatttgaaggaatccaaacgccccgtcaggtaattcacaacatagtgtcggggcbvhaggcggcgttccaagctatbvhtaagcagagcaggcctgtgagcabvhtactctggctccaacccaaccttggtgatcaggaacgcggatccgggcgtcgccgggggcgcgaccctgtgcttgccaaattgaaaatttagcaaagtattttttttaccttgttgattbvhttggataaccgcbvhtccctaatttgabvhatattgctccttatctctaaacgaccaactgtagcttgttctattgaaaactcatcagaataatccatagagataacatcaccaacaaggactgaatcatccagagbvhcatcabvhgccattggagctagggaagtcggcctaactaccttcaacccgacttggccaacttgtacagtagaagacaccbvhgaaatcgatttaacggactctaagggagtgtttgaatacactaaaactaatagttttagtggctaaaattagttggagacatccaaacaccctagctaatattttagctattaactatttttagtaaattagttacaagttagctagctatttattagctagctaattctactaacaaatttttagctaactaacgcattcaaacactgtctaaacaggtaatctaggcacgcgaagtctgactggccagcccaaactcagcctttgtagtagagagagccgaccttggcggcctgcacgacatttttgaactttttttatcggtttggtcttcattactttatattctttgtcaaccaaatactcccttcctcttaaattataattcgtttgattttttttbvhtaaagttttbvhtatagtccacctagttaactttaaaagttggttagtctagattttbvhgaggccgactacaatagcaaatctaacaataaaaaagttagctagatcagatttcaagaacagtctgaccgattttgaagatttgattcaggttaattctacgattttgaagatccgacctaacctaattttacgttcagactaaagacbvhtttgtttaagattbvhatbvhtctagttatataatttaacttattttaaactaacacttaatttaaaataagttagattatbvhatcagaatattatatttatataattccaaacaaataactctaatatcgattctggtttatattgtataatttttbvhttgaatattacggcgcgccaacaaattgcgatcgttbvhtaatcbvhtatacgaccgactgtactattcataaaatataattcaaaataggagbvhctcgagatagcctggcaagggaggaggtatcggaggcggcgtcgcctgccgcgagcgcaccbvhcgtcaacgctcaggacctctcgcaggcgcgctgttggatcgtgagcaagcacgcgtggcaabvhgcbvhcattcctctcgtgcaagtgccgagctgagggtcccgacgcggggggcaggacgtggcagagcgccgcccaggcggacgccaagtgccgagctaccagggcaccttctttattccgtccttgctcagtcacacctcgctctcgctcactctcgccgtccgcacagccgctcatcgtctcccactgcctgccctctccctgcgccggccggtgccccgacgcgccbvhtcgtac

SEQ ID NO: 123 - DNA, 1264 bp, Zea
tcatcggtactcgcgbvhtcgtgcgcgtcgacgactactatatcaaggactbvhacgtcatcttgacaccagaggtgacacagaggaggaccaacaacaataaccactcgccaccgtcggtcggtctgcctttaattctcttcgcaaacacatacacttgcttttttgtttaagcaagcgtgtbvhbvhgtgcgtacctgbvhactgacabvhtacgagggaacttctaccggcaggtgtaacacgtgctcttcabvhataagatcbvhcaaatcgtgtcatatatcgaagcctgcbvhatactgbvhgttgaaatatagtagtgaaacagctaaattaaaataacaaaatttbvhtbvhgctaggattacaaataaattataaaatatttttttataacaatataagacacattttgtatataagttattatattattatbvhtttctgttgcaacgcacgagtactcacctagtatatccataggcttttgatcctctacgtcgagtttttgcttgcbvhagagtcgacctcttctcttttatcccttcttctctatcttagcatttgctcgatcaactgtaaactaattattatacgtacttcaagtcatattgaaaccabvhgcatttaaaaaagtaaccatctgabvhgaaatatcaagctaaataaagaagttacgggagtbvhtgctgagaaaacctagacacattcagagaagttaccaagcaagccttaaataacaatttttggtgctttattgcagatagatactcctataggttggagagaagggcggcgbvhctabvhcaccbvhcbvhtgtgtctgcccatatactctatcttatctgaatcctaccaaactctttaatttgactctttbvhabvhgaattcccatbvhaattctataacacaacagtacgacgtctactggagcatactgccaagcacgctagccgacggctcggcgaccgtctgaggctgtccbvhcbvhcaabvhtgcacacbvhtacgccccttttagcgctggcbvhcacccacgaccacgacacggctcccgtggccgtgaatctgacatcccgggactcccgcctccggccgcgcgtgccgtgtcagcctccatcgagcggtgtactccbvhcaagcctcgacccgggccacgtacccccctcccctccgctacgtgtcaccgctctcgtcacctatatbvhgcggtcttgtccagtctttccacttaactactccgtcactttgtttgcaaag

SEQ ID NO: 124 - DNA, 623 bp, Zea
ctcacacaaatctaaatagtaaagtagatctggttacagagcagaaattttgaagtgtcatagataggctgagaatbvhtagtcacgagcaacacattactacttgaggccatcaatactgttttttcttctccaacctbvhtaactbvhttattcttttcctccttaabvhtaaggcaaacctcctatcctttttctttcaaattcattgcattaactgagtggttcttacbvhtagaabvhaagttggagcaaagaggcttggtgcggaaccgaaaatttgttagtctataaatcattagagggcgcctgaagttcgccaabvhttctagtagattttagtaaccacccacgctagctgactcgaccaccgtctgctggctggcgctgtccbvhcacbvhtacagcccctggcgctggcacgcacccacggcatacctcccbvhactctgacagcccgggactccctccgctcgtgccgtgtcagcctcactcgagcggtgtactccbvhcaagcctccacctcggccacgtaccccctccccttccttacgtgtcaccgctctcgctccctatatacggctgcctggccaggcctttccacttcactactctctctctct

SEQ ID NO: 125 - DNA, 1950 bp, Zea
ggttcaagatbvhtbvhtgbvhaacataattttactcaaaagtgbvhgtttcattgtactaactagcatcaatacttbvhccagagtggagcagtgtgtctbvhcactactccgtacaagagacbvhtccctcacgctacaaggagtbvhggatcccagttgactggttgtctgattccggagtagttggcaaggtgtgcatctctagcatacactccaatttcgccaaacbvhtgttaaaaacbvhcatttttaattccttbvhaaggtgggtbvhattttactcagagtbvhcacctaabvhaactcaataatttatttgcbvhttggatatatcaaattggcatctgttcagcttgcgaacaagtatbvhaagagggttgcatcagaacttgbvhcgttggaaggcactgagaaagagcccaatagagagtttttgcttctccagggtggcagggtgtgaggttctctttccgtgttcatcaggtaaaatcctcacgccaatattttcagaacccaggtacttcacaggtgatbvhaatatttcgaatcagtaacttattcatctaccttctgcagtttgctgggggatttgbvhcagaaagcbvhaaagcttttgaagagctaagaagcaagbvhagtacacataaatctgctccacagatatctgaaggctgaagcbvhagttgaacacaggattcaacacacatctggtcttacttgctggatactgatttttagttgtgtacaaaagagaagtttaaattccagtgtggtgtcaagcaagttgtatbvhctaagagaatacaagtcatcaggttbvhttcatttattttttbvhaaagtaagggcaggacaggttbvhttcbvhcatataacagttttagctcaagtgtataccttttcattttaaaggctgtaactgtaaacaggcagctccagaaabvhaatcacttgggcbvhtttttgttgctacgcatatactactaataaabvhtcaaaagcgtccattcggttagtccggtacccbvhgagataattcgtctgabvhaaccttgccgtctttgttaaaagaaaaaaaaattactttgtgcctcaccgcattcgatactttgaagaaattgacccgttaccagttaccacccacgcacatacagtgctagaacagcagatctgacttgaagaagcatatatcaagacaaaagtccacctgaacggagacagtcgaggttgtggcggagagctccacctgaacgccgaagtggaaccgaacagttccagtggttggccgtggcgagctggccacctactagtctccggggagggggctggctccagcgcctccgagcgcggtgccagtgtcgccgtcgcgtgggttccggttccggccgccgccgcttgccggggaatccaaacggcagacggggtcgccgtgaccgtgaatcgtgtttacttgggccttatctaggccacaacgtttgtgactctgtgagcttattgtttgggcccatctctttggcgtgtctgagaacgaattttatcatcaagcccacgaagccaaaccaggatcctacccacctagcacatactagtgaagaaggtaaacaaagtgtattttcbvhttaattcacbvhcgcgtggtcgacaccacaccacacgacttaaaactcggtgttbvhttctacgaccacaagagccbvhttaattttaagccgtgtctgcgccbvhatccgaagagcgcgtacagtgagagaagaaacgcacggccgccagcagctgccgcgggagcgaggcagcacgtccgttggtcggcccgggcgggagcgaggcacacgtcgcgcbvhcacgcgcggcgcccggacacgtgccggcgcgggcacgtccctctccgcgccttatactcctccccgccatttcccagcagccacaacaagacagagagatacccga

SEQ ID NO: 126 - DNA, 2112 bp, Zea
ctaggttggtgagatccttaggttttggaaggctttbvhtggcagctctgaagactccaacatctbvhgcctctagctbvhcctccatbvhtattttgtaataagaaaaatcatctcccttaaacbvhggagagggttcatctccatcgaacatctttctttaggtggtgaagcctaaaatabvhagcacgacactttgatactaattgaaaggattactbvhcccaagaggtagggtggattagaccaatctaaaatttcttgtagcaataaaatcttatcgaatatcccacttcaccacttgtgcttaagagtgatatatattctatcacaaaaggttttacaccctagtttgaaccctattctagtbvhttagttctagtabvhtaaagacaagaattaaattgctaaabvhcaabvhcttaagataaagaagggttagaaacbvhacagcatttttccaaagtattaaagagccgggactctccaatagtcctcgttggagcacccacacaaggatattgctccccattgaacttcacaaggatcaagggctctctbvhggtttgttcctcgccactctagcbvhgtgagtcacccacagttgtttacaccttgagtggtgatatacactagatttcttaagtgtgtatcatcagacttatttaggtcaaactatcattcbvhgcccctttatagtatagtcaaaggaaaaaacaaaggtgctatcactagtatatbvhtctatcaactctattaactattagaactagccagtccttaacctttttgctcatccttctaaaatcaataattgaataccatcaataggggcacaaaaactatatagccaattattattactcatagccaataatttagtgttgtcattagtcactaaaactagbvhctttcatatctatcbvhctcgtgtttgcctgatccaattcctaggtcbvhtttgggctccacttagagcttgtabvhgcatcacaacbvhatctgattaagaaabvhttgactaaagaccttctcacccacatataaabvhccbvhacttcgctcaaacttcatcccctagtctcctactacacccbvhtctbvhcttcgcaacttcacaagtctaccacbvhtacttgcaaagggctgattctcctagctcccbvhtcgtcgccatctttcccctttaccaccacaaaatcacaabvhtgctcacttattatccccgcgtcttcccttccctaccctaaacactbvhggcaacaacgtcgcatcacttggtggtccttgtagatctagacgaggtctcctccccgttttgtgcttgttgatatagtgatcttgtccttgtcttgtcgctgtcctttggagtcgtcgcagagaggcggtgaaaattctagccgcttctagaaggtgcggtcgccggtcggbvhagagtcgagtgagtgcaggcctataaaaagtagcaattggataacattttgaaaaaabvhaaagaaataaaaaaactagggttaaataagattctaaattaaatctaagaaatctatagacattcggaaatctatttatcttttgtactcctttgattggatttctagattttgacgtttgtgtaattgggcttgacagccccatcaatacaggcaggccagctgatttcgggtgccgctgatttggcccbvhgaggatagagcaggcctttcaacaggcaggcagtccbvhacgtagtgcctcggcctcaaagcccbvhcgattaatatccactggtacgtacgcgacagccgagggagtcactgagtcagtgccgaacggcctggaaaccccccgccgtgtgccggtgtccacgcgcgccccggtgcggccgccacgaccgcggacacgtgccgggcctcgcacacgcgtcccccacggccccctcgcatcattaaaaccgcggtgcccgcgccacggatccccggcacttcattccggtttccggatagcttacctcccatcgacggtcggtgagccgttggtgagtggcgagacagcgggtgcgctgggcaagaacaggcgacgaagaagbvhagcacgccggacacgcggacgctcaaggacgagctgacgagc

SEQ ID NO: 127 - DNA, 1106 bp, Zea
ttggttttttgataatttgtttatctracbvhbvhgttctaacaatbvhcgcagatcttataratctctctcgtagtttttrataaactargagaaagatataggttttbvhaacaaatttarttttattttgtratatabvhaabvhgtraaaatataaattgtacatcbvhbvhagttataraaatttgtagtccaaaatcttttratttaaattaatttartgctttaaabvhtgatttttaattgtctttgcctagtgttgaaaaaaagcactcggcaaagaaactctttgccgagtgttaaaaaaacactcggcaaagaatctctttgccgagtgtraaaaataaaatatttggcaaatagcttctttgctgagtgtttttttgcttagcacttggtaaagaacttctttgccgagtgtccgaaaaagcactcgataaaatarttggcactragcaaagaaccgaattccagtagcgtgagtragaagttggcatttgaaccctttgaaataatraaacaaatraacgcagttgcatbvhaattggctttgccacbvhgtagtgragtccatcgagtgttccbvhtttracagcttaaactaacccbvhgttttggtaacagaacacacaccagtgactccatcgttarcggattttgatartaractgaaggttccggaacaaatartggattctagattgattctagattgcttcgatraabvhgggatratcaacctaacacagcttaaattatacbvhcbvhcgtaratttggtccttraagaggaggcaggtgbvhatcaaggtctctctagcggcctggtgtgcagccacgtarccatccacgcgtcgccgcgggcgacgacaggtgttggccaacctggacatragacaagcacgacabvhacaggcgttcgccaagcgtacgtaraggagacgaacacgacaagtagcaccgtcctggccgagagcccgtcbvhctcbvhcbvhcbvhcbvhctgatcbvhcatartcgataaatataragcagagagctgagctargtacacaaacaagaagcttgcattgttcgttgctcgatcgttgcaggtabvhgaga

SEQ ID NO: 128 - DNA, 922 bp, Zea
ttagtagagaataacacacatctccctaagtatracaaaaaaattaattttaagaggcaattttagtttccaaagcgatttartaagatttaagggagggtatttartagaaggaagtarattgatraaacagagaaaattccatrattbvhgtcccccatagaaatttgtcactcgattgtcgcaaaaaaagttaccagtaaatttgataaattttggataccggtctgttttaactcatcaagaatagcacgacgaaataatagattgcgtacgttttrattcataacatctctractaaagcattttttaracacgctattaaabvhgttcggaaactgatccttaagcggtctattcatatccatctatcgttctaagattcttcctgtargtagattctttccgattaaattctcbvhaaaaattaaaaacaaacaggtccttaaactagtcgccggaagttctcgcttttgctgtagttgtagtgtcgbvhcbvhggtggaaacgtggacagatagattcbvhaabvhtggacaacccacctggtrragagcatcbvhatacattgataracgtggcacgctgcccactgbvhccctragctttgaagctaacgtgtcaagtgtggccggcccctgcattgcgtcgtgccaatcgtccaagtcccbvhgcaaaaacccagcgctttgtgccgccgccgtccgccggcccctctgcccttgtargtgcacctagacacatcgtratcgatcatracacgcaatcgacacaagaagttaataaacagcccaaggacgcagagatragctgatcgagaaggacttgtartactactcagtattgtcgtracbvhcacatatbvhtacataaagagctagctarctgagctctacccaaggtcgcgttgatcgatcgatc

SEQ ID NO: 129 - DNA, 1945 bp, Zea
gattcagaacatctggtcagcttaabvhbvhtggtaaagggagcattattcttttattttgttctttgatctgtgcabvhgccatatartttcgtattgatbvhctctragtggaacagtctgaagctabvhtgaaacttcgbvhttcaggtbvhatabvhtaccaacagagatraaccggctgagbvhcagagttaactarcbvhcattaaagttcctarctgatattgaggaabvhgctgttaagcttgctgcaagabvhaggaacaaaactggcagcataaatccatarbvhtacgttataattatccttttcgctgggtgatartgtgtaragtagagcttgtatctacccaagacactarccagtgcatagctgaagttgacgctctcaaagtttatttcacaatcaaatartgacacattttttttgttcttaaagggctcttcacctgagatttgaaaaagggbvhgtggggctttctttctgtgattttgrcggcacacgggaggagaaggcabvhbvhgcggcttataggcagaaagabvhgccaaggcgcttcaaggtgacgcggactggactagataratctgttarattggactgagaaagaatccctaggagaabvhaagcaccgacactaactatargcgctagttcgggaaccccgttttctcacgagacttttarttttccaagaaaaattagttcattttttttaggaaaataraaatctcttaagaaabvhtagttccgaaactagccctacaagtctaacgattttctcttttttgtarctgccacgcagabvhgatcccatctbvhgccactggcgctgcagaagagaaaagaagggcggtgccctcttgagcccggtgagatcgctgtgatcctgcgggcactggggtaracgagcggcacacagatatargtcgcgtccgggcaagtgtarggcggcaagaaccggbvhgctcccctcaggaacbvhttccccaacctggtaggcaggctcaaatccacttcagattgtgtraagctgaagccagtgataactgataagccactgccgtgcattgtgcatcgctgcaggtgacgaaggaggagctagcgagcgcggaggagctrrcgccgttccggcggcacgtgacgagcctggcggcgctggacttcctggtgtgcctgcggtrrgacgcgttcgtgbvhacgcacggcggcaacttcgccaaactratcatcggggcgcgccgctargcggggcaccgcctraagtcggtgaagcccgacaagggcctcbvhtccaagtccctrrgcgaccccgacbvhggctgggcctccttracggaggacgtcgtcgtracgcaccgcacgaggacgggcctccccgagcccaccttccccggctargatctctgggagaacccgctgacaccctgcbvhtgcagggcbvhagacgggccatccctragatragagtggccgtctggccgagtcctcgtcttcgctaggctargactacgagtagtctcgtgcccacttccatcagcgcgcatttragcgagcatbvhcttctartagttaaacacctagatttgtbvhtatttggctggcctaatctgtagtttcacatagatracttcbvhctgaaabvhtcttggacaaggcagtgabvhaagataacatragtaacgacctgcttttccaaagctcctcttggatttarcaagctgagccaagaabvhgctaacctgagttttgactrrcagccttgcaggcbvhacgtgggcgacctgaactcgctagtcgtagagtttctttccacgcggcagctcggcagagctargcagcctctctgrccgccacgttctcctgcaaggctgcaactcgaagcactgagaagctartaattabvhgattctcgagcbvhcggccttgttcgctaaacct

SEQ ID NO: 130 - DNA, 512 bp, Zea
gccagtgctabvhatatttaractagcgggctgctaaagaaaaccgccagtgctaaagatarttacactagcggttggtgaacaactgcctgtgaaaaaagccgatttctartagcccctagctagcactggcgacataaaaaacgtragtgaaaatagctctaggatcgtractatagagcttctbvhtacttagtggttagaactgatattgtagtgcaccaagtgccgattttaattaaaccaatartaaatartagtaaataatartagtggtctgaattcgatttctaragtabvhtttgcttgcaagccgcaaatagagtaaacattcgtcgtracagaaatccacattaratraaggtccbvhgcggccggccacgtacccatrccacgcgtcgctgcggaggacacgtgttggctgaccggacagttggccgatragacagtggacagaccggacaatagaagaagaagacgacgacggcggcggcaccgccgagtaggtgcat

SEQ ID NO: 131 - DNA, 698 bp, Zea
gtgattaagttgactggcaaattgcatagacgataataractctbvhcttttgagataaccactgtaraccaaccgacttgttgtttgaaggctgatctttaaccattactartgtaactaactgttgttgaataaactattratacgacttggagactttgbvhtatattbvhttttgtarctatactattaactaccgcagatacgtatttaratctcggtaacatctgccagtcgcacaabvhcactaaggtgactgacagactgtagcgaccgatagagcgctgccttgttctagtraaaattcaagcctggtggtcttctcgatccctctgcgcgcgccctgctcttttatcctgtgtgtctcctccacgagggaaggcgtcbvhagaggacgacacgacgaccbvhcagtgctgcgcaaccgcgcgcgtctgcctgttcactgagccccaggcggccggtccaagctattarcacgtcgcccccctccagggctgcgggcagcaacgtgtcgaccggtccgcctctgacgtgtccctcccgtcctctgcataaaagggtgcggctggcggggagcctragcttratatcgtcgcaagctctccgtgtttctctcgtttcttractgctgccaactgtgctgtgcagtagttgcaagagctgattggtagctaggtargctaggcaagtaggc

SEQ ID NO: 132 - DNA, 1355 bp, Zea
tataaattagaacggagggtbvhtttttttargtttccattatttgagtctgctcatracgtttaatttgtttataragttcabvhagttragttaragcggbvhataattcgacggcgtttarttgcbvhcacttartttttacattargcctagabvhtaatcggbvhgacgaatartarttgttttgtgtttattttratatttttttctcttcggatacggatattarcgagttgtgccgaacaagattttgabvhtatatcgacatcttaaatatataactttgaatatarggatargtatatagatcggatattgaatarccagctagactragattaaattggatcttagttattttagggggtgtttgabvhcactaaaactaatagttagtggctaaaattagttgaaacatccaaacaccctarctaatagttragctattagttatttttggaaaattagttaatagttaggtagttarctgttagctagctaattraactaattataagttctagtgcatttaaatarcccttagacggatcgaattagaacaccgbvhgataatatccataacattttaracctataaatbvhtcctarbvhtataragbvhtraaaaacaggttgggcbvhttggbvhacccgtggcacgataragttttaagcagtgccaaacbvhgcbvhgcggcgagctagacbvhatarggtccggttttatttttgtatttttttttcgtaataatarctgtattbvhcttgttcgaacctcacttgcttbvhtggbvhtgatagtatraactcgctggcgttccgtgcatcgtaacatctarcacatcccacagatcbvhtcgcttarcttttctgatartatagcagtagcacacagbvhccggtcgaacccagaagcatcacgccatraccccctccccttttcccaatraaaaccacgtccgtcccabvhgtaacccagcaccaatrrcgtgttcgtgtgagacagagcbvhcaaacaaacagcbvhcbvhcgcgaaggaaagcgtgcagcgcgcgggacgtgaaccgcgcgcgaaccctgccgcccgtccggtctragctcacgcccagcgtccttcccaggccgcgatccggccatcctgacgcgcgttrrgtctgggccccaaaaccacgtccgccgcggcggcggcgcgcccacgggctcccgtgcccgccgatagtttgccggaccctgccgcctcctataabvhcagagccctccgccagcgtctcccgcatctratcttcttcctccatccgcactartgagagcgacaaagctagctctctccagggatcggattcggatracc

SEQ ID NO: 133 - DNA, 1941 bp, Zea
aagggactcgtggcctaracacgcgcacaataatragttaccbvhaattattttarbvhcgaatatcgacattaracbvhaatabvhtgaatttgaatttccgcagcbvhcaaaattcccgtgagtttgaagggcgataaaaacgggtggccaaaatrggggcggcabvhcaagaagataagcctatccatcccttgcattggtartgtratctggggcagggcaggggagggcaggagaagctarctartggccatctatccagcagccgctctccccagccccactctctccatctagagctccctaactgcaccggccgccacaccaccgtarcccgacccctcgacaacabvhgccaccgtcctaggcagcccccgcgcgccggccttctrrttctcgccgtcctccctccgtgccgcgccggcgcccaccgccgtggcgctgcctgcggccaaggtgggcatcbvhggccgtagcgccagcagcaggggcaggctgrgcgcgcaggccacctaraacgtgaagctgatracgccggagggggaggtggagctgcaggtgcccgacgacgtgtaratcctggaccaggccgaggaggacggcatcgacctgccctartcctgccgcgcggggtcctgctcctcctgcgccggcaaggtcgtctccggctccgtggaccagtccgaccagagctarctcgacgacggccagatcgccgccggctgggtgctracctgccacgcctarcccacctctgacgtcgtratcgagacgcacaaggaggaggagctraccggcgcataatrattcbvhcccatccatctctatctctgtgggtcgtcgttgtgtgtaatttgagtargcgcgtaracgtactgcttttgtttcatttgagcactgttcgtgtaagcgtcgagttgccctgccatcgtctctctctctctcgatctctartatttctgtgtgtgcgcgcttctatttttccabvhttgagttargcatatttgtccagtaaccttgttttgcttaatracacacagctcccaaattccagtcgttctgtggtgacggttggttgtggggtgtggcggcgacgtragtcbvhggagaagagatccgabvhcbvhggggtgttggcgctttctctgtccctctccctctccctgtcttctcgcgcgctcgccgtcgtcbvhgctcgacgaacagacattractgccttccccgcgcgcttcagacagaccacgcttgtargtagcgccgcttctctagtcgccggagcaggcacgagtgggtggcgaagaggccacggcccgacctgctagcgccagcgaccgcatrrcgccaccagccgctgcccgctggccgctgccaccgtbvhttgggttgggctgaagcctgaagcccgtgacctttttctttttctttartctttccttttcttttgccggacaagacaagtgbvhttgcaaaatccatataaaatcctagagaaactataaagtttttttttgttgaactccttataacaacgtctarcctttaatatbvhgtatataatttracbvhttttagtataaattgtttartgtatttctbvhtctatbvhctcttggtaagattgtttataaattttaaatttaaaatcttaaaagtrataaacttatagaaaaaaattaggatcctaaacaattttaaacaaaaaaaaaaacttttcaattacaaaatcgtbvhtgttctttbvhcbvhcbvhcaactgagaagctgcccaacggccagcgcagcagaagggcagagcggtgggcggccacgtcgccgccgcaaaggaactgacgtgtcccggcccgacgacgacgacgcgaaccttraccgcgccgcgctccacctctcttaagctaaatargctagtgcaggcgcaggcatccagctgcgacaacgta

SEQ ID NO: 134 - DNA, 1188 bp, Zea
gagcgacctcggactragcggctccgcctgtcggcgagcgcacgccgcgtgcctgrccgcggtctctcgtcgtctccagaagbvhcgccccgatartcgtctttccacccttcttctarctccttccacccccttctccccacgccccatcggatcgcacgcgcaccgcaattgttgttratcacctctccgaagaagcgtracagagcaccgcccggaggttccccttcccgtcgccgtcgcttggccaggtaagtabvhactaatcgcctgagtctcctgcgcctrattacttcgaactgtggtttcgttagtcggacgtcggagcabvhctbvhaaccttgrcattccbvhttarttggtgctgcgtgtccacattcgacgccacatttttttcttgctagccaaccattgcgcgtctgtgttaragttccaccggcgatatarattbvhcgatcatttgbvhtggatartggtgtttttttggttagcggatraccttagttttgcactgaacbvhgaacaabvhtggbvhttggtgtttttttgttagtggatraccttagttttgcactgaagbvhgaacgaggaabvhgggaaggggaactgaacgaagcacatagtttragagactbvhtgcactgragagactbvhcgabvhgtcaagggaatttctcgccttggtttcctaatcctcgcttccttcttgtttcgaattcggtgtaratbvhtggtttartcggcacgtttaggaatttagttggggabvhgggcgcagtbvhtcagggttgtgtratctgaataratabvhcaagatctartactagtttaaattgacttartbvhcaagtcgattaacctggctgggctragtggtgtarcbvhgaagctttgtgattgtagccgggcagtagaacgctaaacctgaggtttggtartgtagcactcggctagtragctgtbvhccaagbvhttacttartcgatactttctgccbvhtctratctgtgttaaccagggtgtgtgccacatagggcttctttccbvhctgtttgttaacctgttracacttcttccataratatattcataatagctcgatctacaattttcctgtcttgagcttctttgttcctaaaaacgaattgtaraccagcabvhtatatctgbvhcaatattgtttgtagg

SEQ ID NO: 135 - DNA, 1507 bp, Zea
ctacatttbvhttatagaggcgcaagtagaagabvhtgtrataagttgtaratcggaaaaatagcbvhtaaatctaragaatraattttcatctttraccccbvhaatttgaggtbvhcttarbvhataactttagaaagtrgtggabvhtcatartctaaaaaaaatagcctarttcattagtaagattccaattcctrgaabvhaaagaaaacaaacggggccttartagabvhgaattraattccabvhatccaabvhgggcgtaagggatttccactttcctaagaaaaatragctcattttccctrggtgtttaataattbvhttttgrtaaaaggtragcttcccgagabvhcgttgtgtaaaagcccagtaraagggtctgtttggttgggctgtggctgrgaaaaaagttgctgtgggctgtgagctgtggaaaaagctgctgtagactgtaagctgttaaaaagctaaaaaccgtttggtgraaaccactaaaagtrgttaaaaaatcttcgatatbvhttttracagttccatccgaaaagccactaaaagcaggtccagaggtgrtttcagatttgcactargagaaagtrggtttttagaaaaagctgcttcctggatccagccctttggktggcttttggctttraggggcacaaaagccaaagccaaaagtraaaccaaacacaccctaagtcgcgtgagtgggccatttarctattccctbvhcaaatctcctgaaaaatracaacatragctcgtraaaaagtaaaataatttraaaaaatragagactaattarttgataactttatagttcatracattttcattgaaabvhttttartttttttgccgaactgatctaracaatatttgaatctaracttttggcttgggctrggagaaaaatagtgcgtgcccagccctttgctctttccccgataacaacgcggtbvhtggcctaacgcccaaatccggcccattrattccggtgcgtagbvhcagacgcgggagcaggtgccaagtractggctcactgrtgcaggtggagtggtggacactagtgccgcggttratctgacacgtgtcgccacgtgccgccbvhgcagcacctragcccggccggcgggccgactgacgtcttgggcaaagcggcgagcgacgcaggcggcgaaagccatccgatttgacccctcgctagacctttcaagaacgaacgctgtgctgctragatcagaccgtgtctgcctraaagcgbvhccaggacgccacgtccaagcaaagcacccgbvhccattgccacctcccagcactracgcgtgagcgtgactataaaaaacgcaccctctgcatccgcccccgtctgcctgccctarcgaatctttcgccgtcccatcagcccagcaattcttcgctgrtcgaggacccctcggtttcgaccgaagcccagcaagccgaccacacaccgctgrcgttggttccgtcccaagagbvh

SEQ ID NO: 136 - DNA, 910 bp, Zea
catacgatttcctaagcggaatctcgagacgaaccacatttttratccggtctbvhagcgcgctraatcgtcagtgacagagaacactgtaggtccttaactggaactaacaattragaaacaatcctaabvhtttartaractggbvhgattttarttggatagbvhatactaccgttcgtgggctaaaaagcaaggtaaaaactggcctaacgcacgtgatrrtcccbvhgtctragaagaaaaccactcgcaaaaaaaaaaacgcagcabvhaggctgctttragttgacggtgtgcacgactcgctgaagccagcgtgattttgtcccggttctrggagcccagcaaaaagggcgcctgaaattccacaggttgaaggggaaaacggcaacaaaaggggctrrgacbvhcggtgcagaaagtgaaaacgcgagaaaagcgcccgtcctttctcgttcccttccgtgcagtrgcaagtgttgrcacgccgcgccgcgacacaccacgcaggaggcagcgagcgcaatabvhcgcacgcagaggagcacagcggcgtractgctgrggcagtabvhgccgcctggttccacttccaccacggtgtgcactarcgctacaccgaggccaccttttrrtggttccttraacacgcctartccggcctccggatcgggcaccacgtctgccccctgctcccggaatctttgccaagcggcgccgctartgtccggcaaggcgaccccgtctctcggccggcgttgatctggtgbvhcgcttttracggtgtttttttttttttttgtttcactcccccctccgtcctgtgtrattgtgctgcagttttgtgccggtgctaacggaaaccggaaagtttgcgtctttbvhttactgtggttraggcgggcgg

SEQ ID NO: 137 - DNA, 1131 bp, Zea
ccagccatcgtgcttgagtgabvhtttttttgcgtgtgtgtaggtgtgcgtgtgtgtttaattgatataaaataacccaaccaaacatctcctaagatttccataagacagagaggtgaggaatagtartagttctraabvhaattraatcaactaaatatraccatcatatcaacattaataatagaacaataaaattttcbvhatagagaggctggtaaactttartttttcatttgattccagtgtggaaaatagtaatataagcagtaaattttttcttaaaaaagagtggaatartcacctttttarataaatragttartcttcgaagcgtracacacggbvhtcctaaagatattcttraactgcaacacaagcattctraaaaggttagtaacaatragattabvhttgtaaaaatbvhtgacacgaaaatttaaagaacgcgaagtacaatarattggtaagaaatraaagacacatattaaccaactartagagaacataracbvhtttcttctaatcctggttaggacataggacgagggtttracttctatracabvhtgctgcaactttragagttaatcaaatctgttattragtgttbvhcagcgacaaattccaabvhtataatcccacgbvhattcbvhtgactccatttcgtrrgtaattgbvhcgaaactgccgaattgttartgctcccataagttaattgtarcacgcttgbvhaagacgttracgtarcttgtgttcgatttbvhtagccgttartcagaccattgcagcgctgagcttraaggcaattagttttgccccgcgbvhtgaggcaggtgbvhacacgcgccctrrgggccgbvhccgacgcgccaccgtgcagactctarcgaggbvhagagcagcgatcgagagacgtrragcgaggaggagactttaggagaggagagaccagagccttaagcggtgacacacccabvhatcaggaccacgcttgcacgtgtraggtgcctctcctccacgcgtcgcgcgcatracactgatactgtcatccagctcgacgctatataaagtggtaagcatcttcgtcctcggggccgccccaacggcccttgcgatrrccaccacgtc

SEQ ID NO: 138 - DNA, 563 bp, Zea
cattgttataratcggtgbvhcagatcttrrcttcgcbvhggtggcaatttagtaacagaattgrtctbvhagttartgcttttagtttctgtctttccccaactgcgtttgagtttgcattgtcacbvhtggccttttttggttgtaagacttgtttgatcggttgtggtgggctgtratcctttggbvhtaggccggaacctcttttcccattarctaaaaaaatacatcgtratttccagtgagacccagcggtcgtgtcatragaaaagcggacacgcgtcgcgtcgcgaggccaggtgggttbvhcacggacctgrtbvhctagccbvhctccaggctccagctccacccgtccaccgacttttcttctraaccaggctgcctcctraccbvhcacgacgacacgtgrgbvhccatcacgcatccacactracctgttgcctctatarccaccgccccaccgccgtagcaccagaaagaabvhtacagtctaracagctgaccagagagctagtgcaagcgatctagcaagtrrtaatcttggaagaagccagctagg

SEQ ID NO: 139 - DNA, 991 bp, Zea
atcatcaccctarcccgagctgactcgggccgcagggaaccagaccggtgrcccatctggctagctrrgccagataggcabvhaaggcgccccgcbvhctctgtgacgacggcggctctcagccccctracggaagcaagaggacgtragcaaggacccaaccgctccgacagctgtccctccgccaggctccatcgctcctccgacggccacgacatcgcaccagctgggtgrcaaaatctctccggctgccacgacggcbvhtactragggcgctagctctcccccgctagacacgtagcactctgctaraccccccattgtacacctggatcctctccttaracctataaaaggaaggaccagggccctctragagaaggttggccgcgcggggacgaggacgagacaggcgctcgcttggggccgctcgctccctctcccacgtggacgcttgtaaccccctartgcaagcgcacccgacctgggcgcgggacgaacacgaaggccgcgggattcccacctctctracgccggtctccggccgcctcgctctccccccttcgcgctcgccctcgcgctcgatccatctgggctggggcacgcggcgacactractcgtcggcccaagggacccccggtctcgaaacgccgacaatbvhatataragaaactatabvhataracbvhcaattaactagtgctgataatatatttaractgtagatttttccgataagtagaaaccaaatarttcaccgccacaaaaggcttcatcccatragttacccbvhtagcaaaaccaaaatarttgtgtgcgagaggccaagagcccaaaccaccgccaaggaaacccccagcccaccaggcgccttcctctaraaataccctaactccggtggccgaagttcctracccattcactcactcatctcaaggcctaggtagcaccgtagcagctartagaagcagctagccagaacaactcgtc

SEQ ID NO: 140 - DNA, 2519 bp, Zea
cttccgataaaaatatttggaaccgcatcctgcatarttatttagaaacattgcatcgataaccgttgcagagcaataracagggtartcaccaactaaatccatarcatacagtgacaggtgaaattggaatarttgatcagcttgaataacagagagcaaagataaatctagccctggtaataaccbvhgctcacatcttbvhtaraagagccaattctaaagacacagctgcgggbvhtagcagtraggcaaggaaagaaacaaaattarcagctaactgctaagtgtraaaacattgctttaractaaagggttaaatarttgccaagaaggtagtgggcacctctcctctgcaagcagcagcataatctagggcgaggcttggtgcagcttccttgtaggggcatbvhaactratccbvhaaggcactctractcatctggacagcagtatagtagctccggtcctgctccaacaatccattttccttracgcctagtttggatractgtaattgaattccattctaataatagtaatttagacatatatraattaagataattracttttgtacaaaatatatttgtatartattattagcaatatatcctaaatarttbvhtgctaratttttactatagaggagtagaacgaagagtgtratataagttatagatragaaacaaattctaatcbvhcataaaatratttcccatcctccaccctbvhaatttgagataggcttatarctgaactttagaaagtggtggabvhtcaaattccaaactaaataagttactttattgagtgaattctaattcttttgatttgaaggaatccaaacgccccgtraggtaattracaacatagtgtcggggcbvhaggcggcgtrrcaagctatbvhtaagcagagcaggcctgtgagcabvhtactctggctccaacccaaccttggtgatraggaacgcggatccgggcgtcgccgggggcgcgaccctgtgcttgccaaattgaaaatttagcaaagtartttttttaccttgttgattbvhttggataaccgcbvhtccctaatttgabvhatattgctccttatctctaaacgaccaactgtagcttgttctattgaaaactratcagaataatccatagagataacatraccaacaaggactgaatratccagagbvhcatcabvhgccattggagctagggaagtcggcctaactarcttraacccgacttggccaacttgtaragtagaagacaccbvhgaaatcgatttaacggactctaagggagtgtttgaataractaaaactaatagttttagtggctaaaattagttggagacatccaaacaccctagctaatatttragctattaactatttttagtaaatragttacaagttagctagctatttattagctagctaattctartaacaaatttttagctaactaacgcattcaaacactgtctaaacaggtaatctaggcacgcgaagtctgactggccagcccaaactragcctttgtagtagagagagccgaccttggcggcctgracgacatttttgaactttttttarcggtttggtcttrattactttatattctttgtraaccaaatartcccttcctcttaaattataattcgtttgattttttttbvhtaaagttttbvhtatagtccacctagttaactttaaaagttggttagtctagattttbvhgaggccgactaraatagcaaatctaacaataaaaaagttagctagatragatttcaagaacagtctgaccgattttgaagatttgattraggttaattctacgattttgaagatccgacctaacctaattttacgttragactaaagacbvhtttgtttaagattbvhatbvhtctagttarataatttaactrattttaaactaacacttaatttaaaataagttagattatbvhatragaatattatatttatataattccaaacaaataactctaatatcgattctggtttarattgtataatttttbvhttgaatattarggcgcgccaacaaattgcgatcgttbvhtaatcbvhtatargaccgactgtartattcataaaatataattcaaaataggagbvhctcgagatagcctggcaagggaggaggtarcggaggcggcgtcgcctgccgcgagcgcaccbvhcgtcaacgctraggacctctcgcaggcgcgctgttgratcgtgagcaagcacgcgtggcaabvhgcbvhcattcctctcgtgcaagtgccgagctgagggtcccgacgcggggggcaggacgtggcagagcgccgcccaggcggacgccaagtgccgagctarcagggcaccttctttartccgtccttgctragtcacacctcgctctcgctcactctcgccgtccgcacagccgctratcgtctcccactgcctgccctctccctgcgccggccggtgrcccgacgcgccbvhtcgtac

SEQ ID NO: 141 - DNA, 1264 bp, Zea
tcatcggtactcgcgbvhtcgtgcgcgtcgacgactartatatraaggactbvhacgtratcttgacaccagaggtgacacagaggaggaccaacaacaataaccactcgccaccgtcggtcggtctgcctttaattctcttcgcaaacacataracttgcttttttgtttaagcaagcgtgtbvhbvhgtgcgtacctgbvhactgacabvhtargagggaacttctarcggcaggtgtaacacgtgctcttrabvhataagatcbvhcaaatcgtgtratatatcgaagcctgcbvhatartgbvhgttgaaataragtagtgaaacagctaaattaaaataacaaaatttbvhtbvhgctaggattaraaataaattaraaaatatttttttataacaatataagacacattttgtarataagttattatartattatbvhtttctgttgcaacgcacgagtartcacctagtatarccataggcttttgatcctctargtcgagtttttgcttgcbvhagagtcgacctcttctcttttarcccttcttctctarcttagcatttgctcgatraactgtaaactaattattaracgtarttcaagtcatattgaaaccabvhgcatttaaaaaagtaaccatctgabvhgaaatatraagctaaataaagaagttargggagtbvhtgctgagaaaacctagacacattragagaagttarcaagcaagccttaaataacaatttttggtgctttartgcagatagatartcctataggttggagagaagggcggcgbvhctabvhcaccbvhcbvhtgtgtctgcccatatartctatcttarctgaatcctarcaaactctttaatttgactctttbvhabvhgaattcccatbvhaattctataacacaacagtargacgtctartggagcatactgccaagcacgctagccgacggctcggcgaccgtctgaggctgtccbvhcbvhcaabvhtgcacacbvhtargccccttttagcgctggcbvhcacccacgaccacgacacggctcccgtggccgtgaatctgacatcccgggactcccgcctccggccgcgcgtgccgtgtragcctccatcgagcggtgtartccbvhcaagcctcgacccgggccacgtarccccctcccctccgctargtgtraccgctctcgtracctatatbvhgcggtcttgtccagtctttccacttaactartccgtcactttgtttgcaaag

SEQ ID NO: 142 - DNA, 623 bp, Zea
ctcacacaaatctaaatagtaaagtagatctggtracagagcagaaattttgaagtgtratagataggctgagaatbvhtagtracgagcaacacattartacttgaggccatraatactgttttttcttctccaacctbvhtaactbvhttartcttttcctccttaabvhtaaggcaaacctcctarcctttttctttcaaattrattgcattaactgagtggttcttacbvhtagaabvhaagttggagcaaagaggcttggtgcggaaccgaaaatttgttagtctataaatrattagagggcgcctgaagttcgccaabvhttctagtagattttagtaaccacccacgctagctgactcgaccaccgtrrgctggctggcgctgtccbvhcacbvhtaragcccctggcgctggcacgcacccacggcatarctcccbvhactctgacagcccgggactccctccgctcgtgccgtgtragcctcactcgagcggtgtartccbvhcaagcctccacctcggccacgtarcccctccccttccttargtgtcaccgctctcgctccctatatarggctgcctggccaggcctttccacttractactctctctctct

SEQ ID NO: 143 - DNA, 1950 bp, Zea
ggttcaagatbvhtbvhtgbvhaacataattttartcaaaagtgbvhgtttrattgtartaactagcatraatarttbvhccagagtggagcagtgrgtctbvhcactartccgtaraagagacbvhtccctracgctacaaggagtbvhggatcccagttgactggttgtctgattccggagtagttggcaaggtgtgcatctctagcataractccaatttcgccaaacbvhtgttaaaaacbvhcatttttaattccttbvhaaggtgggtbvhattttartragagtbvhcacctaabvhaactraataatttarttgcbvhttggatatatraaattggcatctgttragcttgcgaacaagtatbvhaagagggttgcatragaacttgbvhcgttggaaggcactgagaaagagcccaatagagagtttttgcttctccagggtggcagggtgtgaggttctctttccgtgttratraggtaaaatcctracgccaatattttcagaacccaggtarttracaggtgatbvhaatatttcgaatragtaacttattratctarcttctgcagtttgctgggggatttgbvhcagaaagcbvhaaagcttttgaagagctaagaagcaagbvhagtaracataaatctgctccacagatarctgaaggctgaagcbvhagttgaacacaggattraacacacatctggtcttarttgctggatactgatttttagttgtgtaraaaagagaagtttaaattccagtgtggtgtcaagcaagttgtatbvhctaagagaataraagtcatraggttbvhttratttattttttbvhaaagtaagggcaggacaggttbvhttcbvhcatataacagttttagctraagtgtatarcttttrattttaaaggctgtaactgtaaacaggcagctccagaaabvhaatracttgggcbvhtttttgttgctacgcatatartactaataaabvhtcaaaagcgtccattcggttagtccggtarccbvhgagataattcgtctgabvhaaccttgccgtctttgttaaaagaaaaaaaaattartttgtgcctraccgcattcgatartttgaagaaattgacccgttarcagttarcacccacgcacataragtgctagaacagcagatctgacttgaagaagcatatatraagacaaaagtccacctgaacggagacagtcgaggttgtggcggagagctccacctgaacgccgaagtggaaccgaacagttccagtggttggccgtggcgagctggccacctartagtctccggggagggggctggctccagcgcctccgagcgcggtgccagtgtcgccgtcgcgtgggttccggttccggccgccgccgcttgccggggaatccaaacggcagacggggtcgccgtgaccgtgaatcgtgtttarttgggccttarctaggccacaacgtttgtgactctgtgagcttartgtttgggcccatctctttggcgtgtctgagaacgaattttatratcaagcccacgaagccaaaccaggatcctarccacctagcacatartagtgaagaaggtaaacaaagtgtattttcbvhttaattcacbvhcgcgtggtcgacaccacaccacacgacttaaaactcggtgttbvhttctargaccacaagagccbvhttaattttaagccgtgtctgcgccbvhatccgaagagcgcgtaragtgagagaagaaacgcacggccgccagcagctgccgcgggagcgaggcagcacgtccgttggtcggcccgggcgggagcgaggcacacgtcgcgcbvhcacgcgcggcgcccggacacgtgccggcgcgggcacgtccctctccgcgccttatartcctccccgccatttcccagcagccacaacaagacagagagatarccga

SEQ ID NO: 144 - DNA, 2112 bp, Zea
ctaggttggtgagatccttaggttttggaaggctttbvhtggcagctctgaagactccaacatctbvhgcctctagctbvhcctccatbvhtattttgtaataagaaaaatratctcccttaaacbvhggagagggttratctccatcgaacatctttcttraggtggtgaagcctaaaatabvhagcacgacactttgatartaattgaaaggattartbvhcccaagaggtagggtggattagaccaatctaaaatttcttgtagcaataaaatcttarcgaatarcccacttcaccacttgtgcttaagagtgataratartctarcacaaaaggttttacaccctagtttgaaccctartctagtbvhttarttctartabvhtaaagacaagaattaaattgctaaabvhcaabvhctraagataaagaagggttagaaacbvhacagcatttttccaaagtartaaagagccgggactctccaatagtcctcgttggagcacccacacaaggatartgctccccattgaacttracaaggatraagggctctctbvhggtttgttcctcgccactctagcbvhgtgagtracccacagtrgtttaraccttgagtggtgatataractagatttcttaagtgtgtatratragacttarttaggtcaaactatrattcbvhgccccttratagtatagtraaaggaaaaaacaaaggtgctatractagtaratbvhtctatraactctartaactartagaactagccagtccttaacctttttgctratccttctaaaatraataattgaatarcatraataggggcacaaaaactaratagccaattartartactratagccaataatttagtgttgtrattagtractaaaactagbvhctttratatctarcbvhctcgtgtttgcctgatccaattcctaggtcbvhtttgggctccacttagagcttgtabvhgcatracaacbvhatctgattaagaaabvhttgactaaagaccttctracccacatataaabvhccbvhacttcgctraaacttratcccctagtctcctartaracccbvhtctbvhcttcgcaacttracaagtctarcacbvhtarttgcaaagggctgattctcctagctcccbvhtcgtcgccatctttcccctttarcaccacaaaatracaabvhtgctracttartatccccgcgtcttcccttccctarcctaaacactbvhggcaacaacgtcgcatracttggtggtccttgtagatctagacgaggtctcctccccgttttgtgcttgttgatatagtgatcttgtccttgtcttgtcgctgtcctttggagtcgtcgcagagaggcggtgaaaattctagccgcttctagaaggtgcggtcgccggtcggbvhagagtcgagtgagtgcaggcctataaaaagtagcaatrggataacattttgaaaaaabvhaaagaaataaaaaaactagggtraaataagattctaaattaaatctaagaaatctatagacattcggaaatctatttarcttttgtartcctttgattggatttctagattttgacgtttgtgtaattgggcttgacagccccatraataraggcaggccagctgatttcgggtgccgctgatttggcccbvhgaggatagagcaggcctttraacaggcaggcagtccbvhacgtagtgcctcggcctraaagcccbvhcgattaatarccactggtargtargcgacagccgagggagtractgagtragtgccgaacggcctggaaaccccccgccgtgtgrcggtgtrracgcgcgccccggtgrggccgccacgaccgcggacacgtgrcgggcctcgcacacgcgtcccccacggccccctcgcatcattaaaaccgcggtgcccgcgccacggatccccggcacttrattccggtttccggatagcttarctcccatcgacggtcggtgagccgttggtgagtggcgagacagcgggtgcgctgrgcaagaacaggcgacgaagaagbvhagcacgccggacacgcggacgctraaggacgagctgacgagc

SEQ ID NO: 145 - DNA, 111 bp, Zea mays
tgcgtacgagacgtgttggcgacggccgaccggcgaccgccgccgagctcgttttgccttgttgctttgtttgttatatggccctggctccgatccgggcttcgtatttaa

SEQ ID NO: 146 - DNA, 165 bp, Zea mays
gtacgatgctggtaagctcatcagagttcccgtcgttgctaccggcgagaaaccttcttcgtatgacaccggtggccatggcgaacaccccggtgccaccgtagatggcccactcgtctgacccatccgttatcactccgttggtgagcagcgtggagcccttaa

SEQ ID NO: 147 - DNA, 117 bp, Zea mays
tcgattgatctatccatagtctggccagcatatatccgggcaaaattgggaagaacgtggcgcattatgtctgaagtatatcctccccaccagctgtgcatcctgatgatgctctta

SEQ ID NO: 148 - DNA, 164 bp, Zea mays
gacaccttggaacaccagatctacagaagtcctgtgatatctccggtcagctcctgcgtcgtctccacacgctttcactgcacatggtcgtgctcagcctcagcctagcatttgttttgtttcccagcccgacaggaagaaacaggtgggttactcaggactca

SEQ ID NO: 149 - DNA, 70 bp, Zea mays
tcgaaccatatatgtaggctcatttcatgagataaaatcagatgcatgtgagcatgtccatatatgttaa

SEQ ID NO: 150 - DNA, 172 bp, Zea mays
tgcgtacgacgttgctggtgaatgatggtttggcggcgtgtgtgcaaacttttctatgctagtttagtttgcaggtgtgtatggattttgatttatggcctggatggatcacactatgtagcgaacgatgtgttcaatttactgttggaataaaactgcagattggtgttaa

SEQ ID NO: 151 - DNA, 156 bp, Zea mays
ggtacgacggagacggtcagccgtacgcgttccggaacccgggctcgctgtcgtagaggttgtacggctccggcgcgccgcccttcttcttcttgcccttcctgttcgcctccgcagcgccgccgccgccagccgcccctccgacgaccctgttaa

SEQ ID NO: 152 - DNA, 123 bp, Zea mays
tcgacgtcggttcgttcgtgcctccaccttagcctagggttggtttcttgcaaggtagtgagtgtgtcttagtctcaccatcaccggggctccaattttggaaagctgcgtgttaggagttaa

SEQ ID NO: 153 - DNA, 171 bp, Zea mays
gcgtacgatgcatggcacggttaggcctgtgcgtgtgcccatgggcaatcaagacaacttctgagctctggagttgttgttgttgttctgtgttcttgcttcctcgttgagctgtgcccgtttgtcctcacttatgcagtcagtggtctgtctgtagtctgtactgcttaa

SEQ ID NO: 154 - DNA, 25 bp, Artificial
  Primer
ctacatttatgttatagaggcgcaa

SEQ ID NO: 155 - DNA, 21 bp, Artificial
  Primer
catctcttgggacggaaccaa

SEQ ID NO: 156 - DNA, 23 bp, Artificial
  Primer
catacgatttcctaagcggaatc

SEQ ID NO: 157 - DNA, 20 bp, Artificial
  Primer
ccgcccgcctcaaccacagt

SEQ ID NO: 158 - DNA, 20 bp, Artificial
  Primer
ccagccatcgtgcttgagtg

SEQ ID NO: 159 - DNA, 20 bp, Artificial
  Primer
gacgtggtggcgatcgcaag

SEQ ID NO: 160 - DNA, 21 bp, Artificial
  Primer
cattgttatacatcggtgatg

SEQ ID NO: 161 - DNA, 21 bp, Artificial
  Primer
cctagctggcttcttccaagc

SEQ ID NO: 162 - DNA, 23 bp, Artificial
  Primer
gagcgacctcggactcagcggct

SEQ ID NO: 163 - DNA, 22 bp, Artificial
  Primer
cctacaaacaatattgcatcag

SEQ ID NO: 164 - DNA, 20 bp, Artificial
  Primer
gattcagaacatctggtcag

SEQ ID NO: 165 - DNA, 18 bp, Artificial
  Primer
aggtttagcgaacaaggc

SEQ ID NO: 166 - DNA, 21 bp, Artificial
  Primer
atcatcaccctaccccgagct

SEQ ID NO: 167 - DNA, 19 bp, Artificial
  Primer
gacgagttgttctggctag

SEQ ID NO: 168 - DNA, 23 bp, Artificial
  Primer
cttccgataaaaatatttggaac

SEQ ID NO: 169 - DNA, 19 bp, Artificial
  Primer
gtacgacatggcgcgtcgg

SEQ ID NO: 170 - DNA, 20 bp, Artificial
  Primer
gccagtgctaatgatattta

SEQ ID NO: 171 - DNA, 19 bp, Artificial
  Primer
atgcacctactcggcggtg

SEQ ID NO: 172 - DNA, 20 bp, Artificial
  Primer
tcatcggtactcgcgatgtc

SEQ ID NO: 173 - DNA, 22 bp, Artificial
  Primer
ctttgcaaacaaagtgacggag

SEQ ID NO: 174 - DNA, 24 bp, Artificial
  Primer
tataaattagaacggaggggtatg

SEQ ID NO: 175 - DNA, 20 bp, Artificial
  Primer
ggtgatccgaatccgatccc

SEQ ID NO: 176 - DNA, 24 bp, Artificial
  Primer
ctcacacaaatctaaatagtaaag

SEQ ID NO: 177 - DNA, 21 bp, Artificial
  Primer
gagagagagagtagtgaagtg

SEQ ID NO: 178 - DNA, 22 bp, Artificial
  Primer
ggttcaagatatgtatgtgatg

SEQ ID NO: 179 - DNA, 23 bp, Artificial
  Primer
tcgggtatctctctgtcttgttg

SEQ ID NO: 180 - DNA, 25 bp, Artificial
  Primer
ttggttttttgataatttgtttatc

SEQ ID NO: 181 - DNA, 21 bp, Artificial
  Primer
tctccattacctgcaacgatc

SEQ ID NO: 182 - DNA, 20 bp, Artificial
  Primer
aagggactcgtggcctacac

SEQ ID NO: 183 - DNA, 20 bp, Artificial
  Primer
tacgttgtcgcagctggatg

SEQ ID NO: 184 - DNA, 22 bp, Artificial
  Primer
ttagtagagaataacacacatc

SEQ ID NO: 185 - DNA, 18 bp, Artificial
  Primer
gatcgatcgatcaacgcg

SEQ ID NO: 186 - DNA, 24 bp, Artificial
  Primer
gtgattaagttgactggcaaattg

SEQ ID NO: 187 - DNA, 20 bp, Artificial
  Primer
gcctacttgcctagcgtacc

SEQ ID NO: 188 - DNA, 21 bp, Artificial
  Primer
aaaagtagcaattgggataac

SEQ ID NO: 189 - DNA, 21 bp, Artificial
  Primer
gctcgtcagctcgtccttgag

SEQ ID NO: 190 - DNA, 21 bp, Artificial
  Primer
ctaggttggtgagatccttag

SEQ ID NO: 191 - DNA, 21 bp, Artificial
  Primer
catcttcttcgacgcctgttc

SEQ ID NO: 192 - DNA, 23 bp, Artificial
  Primer
gtggcagctctgaagactccaac

SEQ ID NO: 193 - DNA, 23 bp, Artificial
  Primer
tgaggccgaggcactacgtcatg

SEQ ID NO: 194 - DNA, 23 bp, Artificial
  Primer
tgacgtttgtgtaattgggcttg

SEQ ID NO: 195 - DNA, 21 bp, Artificial
  Primer
gctcgtcagctcgtccttgag

SEQ ID NO: 196 - DNA, 1696 bp, Zea mays
aaaagtagcaattgggataacattttgaaaaaaatgaaagaaataaaaaaactagggttaaataagattctaaattaaatctaagaaatctatagacattcggaaatctatttatcttttgtactcctttgattggatttctagattttgacgtttgtgtaattgggcttgacagccccatcaatacaggcaggccagctgatttcgggtgccgctgattcggcccatggaggatagagcaggcctttcaacaggcaggcagtccatgacgtagtgcctcggcctcaaagcccatgcgattaatatccactggtacgtacgcgacagccgagggagtcactgagtcagtgccgaacggcctggaaaccccccgccgtgtgccggtgtccacgcgcgccccggtgcggccgccacgaccgcggacacgtgccgggcctcgcacacgcgtcccccacggccccctcgcatcattaaaaccgcggtgcccgcgccacggatccccggcacttcattccggtttccggatagcttacctcccatcgacggtcggtgagccgttggtgagtggcgagacagcgggtgcgctgggcaagaacaggcgacgaagaagatgagcacgccggacacgcggacgctcaaggacgagctgacgagcatggacaggaagtgcctcgtggacctcggccacccgctgctcaaccgcgtcgccgacagcttcatccgcgccgctggggtcggggcggccagggccgtctccagggaggcctacgtcgtcaccgtcgaaggtaccgcgtcccggccgagtctacctaactagcgagcggctggtgctcacccgcccgttgcaaatctatctgcagggctttcaggagactcgtccgggctagacgccgacggcggcaagcggagccatttctccagcatcagaggtgacgacggccagaggtcgctcgacgcagtggtatgtcacaaacatagctcagagctgcgattcgttgtgcagattcattaatggtttctctctcaccccctcttgacaatttgcaggtaaaaacggccggcaaggaggccttccagtggggttagtggtgtgattagtctgttcagatctaacagtacgttggaggtccacaagtagtataaccctgtttctctcgacctcgacatgcagggttggcagccggagtctactcagggctcacgtacgcgctgcgggaggccaggggatgccacgactgggtcagtgccatttcctagcttttaaccagacggccgtttttggcttggtctcgcgtgtgaactttgctgtcctcgtgcagaagaacagcgccatcgcaggcgccatcgctggggcggcggtggcgctcacgggtgacgccggcggccactccgacaagctcgtcaacttcgccatcaccggcgccgcgctctccagcgccgggagcttgctctccggcatattctgattggcccccagggctgcacccgcctgacacgggttcgctgcccggttgagcccttcatccgaggggttttagcatgaaaggttgatttcaggccagcaatgaaatatgtattcctagggccctgccggtctagcactgtgcgcttccagtgtaatcgttgcttcgtgtacgagtttcagacatttcgaaatatcttctccgagttccggtggtttgacatgtgcaaacat

(19) United States
(12) Patent Application Publication
(10) Pub. No.: US 2012/0240287 A1
(43) Pub. Date: Sep. 20, 2012

(54) Expression Cassettes for Embryo-Specific Expression in Plants

(75) Inventors: Huihua Fu, Cary, NC (US); Jeffrey A. Brown, Apex, NC (US); Kirk Francis, Cary, NC (US); Hee-Sook Song, Raleigh, NC (US)

(73) Assignee: BASF Plant Science Company GmbH, Ludwigshafen (DE)

(21) Appl. No.: 13/512,922

(22) PCT Filed: Nov. 30, 2010
(86) PCT No.: PCT/IB2010/055490

§ 371 Date: May 31, 2012

Related U.S. Application Data
(60) Provisional application No. 61/266,248, filed on Dec. 3, 2009.

(51) Int. Cl. A01H 5/00; A01H 5/10; A01H 1/06; C12N 15/82; C12N 5/10

(52) U.S. Cl. 800/278; 435/320.1; 435/419; 800/298

(57) ABSTRACT

An expression cassette for regulating embryo-specific expression of a polynucleotide of interest, comprising a transcription regulating nucleotide sequence, is provided. Vectors, host cells and transgenic plants comprising said expression cassette, and methods of producing said transgenic plants are also provided.

### FIELD OF THE INVENTION

The present invention relates to expression cassettes comprising transcription regulating nucleotide sequences with whole seed and/or embryo-specific expression profiles in plants obtainable from the Zea mays. The transcription regulating nucleotide sequences preferably exhibit strong expression activity especially in whole seeds and, particularly, in the endosperm.

### BACKGROUND OF THE INVENTION

Manipulation of plants to alter and/or improve phenotypic characteristics (such as productivity or quality) requires the expression of heterologous genes in plant tissues. Such genetic manipulation relies on the availability of a means to drive and to control gene expression as required. For example, genetic manipulation relies on the availability and use of suitable promoters which are effective in plants and which regulate gene expression so as to give the desired effect(s) in the transgenic plant.

A fertile corn plant contains both male and female reproductive tissues, commonly known as the tassel and the ear, respectively. The tassel tissues form the haploid pollen grains with two nuclei in each grain, which, when shed at anthesis, contact the silks of a female ear. The ear may be on the same plant as that which shed the pollen, or on a different plant. The pollen cell develops a structure known as a pollen tube, which extends down through an individual female silk to the ovule. The two male nuclei travel through this tube to reach the haploid female egg at the base of the silk. One of the male nuclei fuses with and fertilizes the female haploid egg nuclei to form the zygote, which is diploid in chromosome number and will become the embryo within the kernel. The remaining male nucleus fuses with and fertilizes a second female nucleus to form the primary endosperm nucleus, which is triploid in number and will become the endosperm of the kernel, or seed, of the corn plant. Non-fertilized ovules do not produce kernels and the unfertilized tissues eventually degenerate.

The kernel consists of a number of parts, some derived from maternal tissue and others from the fertilization process. Maternally, the kernel inherits a number of tissues, including a protective, surrounding pericarp and a pedicel. The pedicel is a short stalk-like tissue which attaches the kernel to the cob and provides nutrient transfer from maternal tissue into the kernel. The kernel contains tissues resulting from the fertilization activities, including the new embryo as well as the endosperm. The embryo is comprised of the cells that will develop into the roots and shoots of the next generation corn plant. It is also the tissue in which oils and quality proteins are stored in the kernel. The endosperm functions as a nutritive tissue and provides the energy in the form of stored starch and proteins needed for germination and the initial growth of the embryo.

Considering the complex regulation that occurs during embryo and kernel development in higher plants, and considering that grain is commonly used as a primary source of nutrition for animals and humans, it is important to develop key tools that can be used to improve these tissues from a nutritional standpoint. One class of such tools would be transcriptional promoters that can drive the expression of nutrition enhancing genes specifically in these tissues. Unfortunately, relatively few promoters specifically directing this expression pattern have been identified. Accordingly, there is a need in the art for novel promoter sequences which drive expression during kernel development, and more particularly, embryo development.

The embryo-specific promoters are useful for expressing genes as well as for producing large quantities of protein, for expressing genes involved in the synthesis of oils or proteins of interest, e.g., antibodies, genes for increasing the nutritional value of the whole seed, and, particularly, the embryo and the like. It is advantageous to have the choice of a variety of different promoters so that the most suitable promoter may be selected for a particular gene, construct, cell, tissue, plant or environment. Moreover, the increasing interest in cotransforming plants with multiple plant transcription units (PTU) and the potential problems associated with using common regulatory sequences for these purposes merit having a variety of promoter sequences available.

Only a few embryo or whole seed-specific promoters have been cloned and studied in detail; these include promoters for seed storage protein genes, such as a globulin promoter (Wu et al. (1998) Plant Cell Physiol 39 (8) 885-889), phaseolin promoter (U.S. Pat. No. 5,504,200) and a napin promoter (U.S. Pat. No. 5,608,152). Storage proteins are usually present in large amounts, making it relatively easy to isolate storage protein genes and the gene promoters. Even so, the number of available seed specific promoters is still limited. Furthermore, most of these promoters suffer from several drawbacks; they may drive expression only in a limited period during seed development, and they may be expressed in other tissues as well. For example, storage protein gene promoters are expressed mainly in the mid to late embryo development stage (Chen et al., Dev. Genet., 10 (2): 112-122 (1989); Keddie et al., Plant Mol. Biol., 19 (3): 443-53 (1992); Sjodahl et al., Planta., 197 (2): 264-71 (1995); Reidt et al., Plant J., 21 (5): 401-8 (2000)), and also may have activity in other tissues, such as pollen, stamen and/or anthers (as, for example, the phaseolin promoter, as reported by Ahm, V, et al. Plant Phys 109: 1151-1158 (1995); or the zmHyPRP promoter as described in Gene 356 (2005), 146-152; or promoters described in U.S. Pat. No. 5,912,414).

There is, therefore, a great need in the art for the identification of novel sequences that can be used for expression of selected transgenes in economically important plants. Thus, the problem underlying the present invention is to provide new and alternative expression cassettes for embryo-expression of transgenes in plants. The problem is solved by the present invention.

### SUMMARY OF THE INVENTION

Accordingly, a first embodiment of the invention relates to an expression cassette for regulating seed-specific expression of a polynucleotide of interest, said expression cassette comprising a transcription regulating nucleotide sequence selected from the group of sequences consisting of:
(a) a nucleic acid sequence of SEQ ID NO: 1, 2, 3, 4, 5, 6, 7, 8, 9, 10, 11, 12, 13, 14, 15, 16, 17, or 18, or a variant thereof;
(b) a nucleic acid sequence which is at least 80% identical to a nucleic acid sequence shown in any one of SEQ ID NO: 1, 2, 3, 4, 5, 6, 7, 8, 9, 10, 11, 12, 13, 14, 15, 16, 17, or 18;
(c) a nucleic acid sequence which hybridizes under stringent conditions to a nucleic acid sequence of SEQ ID NO: 1, 2, 3, 4, 5, 6, 7, 8, 9, 10, 11, 12, 13, 14, 15, 16, 17, or 18, or a variant thereof;
(d) a nucleic acid sequence which hybridizes to a nucleic acid sequence located upstream of an open reading frame sequence of SEQ ID NOs: 19, 20, 21, 22, 23, 24, 25, 26, 27, 28, 29, 30, 31, 32, 33, 34, 35 or 36, or a variant thereof;
(e) a nucleic acid sequence which hybridizes to a nucleic acid sequences located upstream of an open reading frame sequence encoding an amino acid sequence of SEQ ID NOs: 37, 38, 39, 40, 41, 42, 43, 44, 45, 46, 47, 48, 49, 50, 51, 52, 53 or 54, or a variant thereof;
(f) a nucleic acid sequence which hybridizes to a nucleic acid sequence located upstream of an open reading frame sequence being at least 80% identical to an open reading frame sequence of SEQ ID NOs: 19, 20, 21, 22, 23, 24, 25, 26, 27, 28, 29, 30, 31, 32, 33, 34, 35 or 36, wherein the open reading frame encodes a seed protein;
(g) a nucleic acid sequence which hybridizes to a nucleic acid sequences located upstream of an open reading frame encoding an amino acid sequence being at least 80% identical to an amino acid sequence as shown in SEQ ID NOs: 37, 38, 39, 40, 41, 42, 43, 44, 45, 46, 47, 48, 49, 50, 51, 52, 53 or 54, wherein the open reading frame encodes a seed protein;
(h) a nucleic acid sequence obtainable by 5′ genome walking or by thermal asymmetric interlaced polymerase chain reaction (TAIL-PCR) on genomic DNA from the first exon of an open reading frame sequence as shown in SEQ ID NOs: 19, 20, 21, 22, 23, 24, 25, 26, 27, 28, 29, 30, 31, 32, 33, 34, 35 or 36; and
(i) a nucleic acid sequence obtainable by 5′ genome walking or TAIL PCR on genomic DNA from the first exon of an open reading frame sequence being at least 80% identical to an open reading frame as shown in SEQ ID NOs: 19, 20, 21, 22, 23, 24, 25, 26, 27, 28, 29, 30, 31, 32, 33, 34, 35 or 36, wherein the open reading frame encodes a seed protein; and
(j) a nucleic acid sequence obtainable by 5′ genome walking or TAIL PCR on genomic DNA from the first exon of an open reading frame sequence encoding an amino acid sequence being at least 80% identical to an amino acid sequence encoded by an open reading frame as shown in any one of SEQ ID NOs: 37, 38, 39, 40, 41, 42, 43, 44, 45, 46, 47, 48, 49, 50, 51, 52, 53 or 54, wherein the open reading frame encodes a seed protein.

In a preferred embodiment, the expression cassette further comprises at least one polynucleotide of interest being operatively linked to the transcription regulating nucleotide sequence, preferably being heterologous with respect to the transcription regulating nucleotide sequence.

In another aspect, the present invention refers to a transgenic plant tissue, plant organ, plant or seed comprising the expression cassette or the vector of the present invention. Preferably, the transgenic plant is a monocotyledone.

In another aspect, the present invention refers method for producing a transgenic plant tissue, plant organ, plant or seed comprising
(a) introducing the expression cassette or the vector of the present invention into a plant cell; and
(b) regenerating said plant cell to form a plant tissue, plant organ, plant or seed.

In another aspect, the present invention refers to a method for producing a transgenic plant tissue, plant organ, plant or seed comprising
(a) integrating the expression cassette or the vector of the present invention into the genome of a plant cell;
(b) regenerating said plant cell to form a plant tissue, plant organ, plant or seed, and
(c) selecting said plant cell to form a plant tissue, plant organ, plant or seed for the presence of the expression cassette or the vector of the present invention.

Other embodiments of the invention relate to vectors comprising an expression cassette of the invention, and transgenic host cells or transgenic plant comprising an expression cassette or a vector of the invention, and methods of producing the same.

### DESCRIPTION OF THE DRAWINGS

FIG. 1: q-RT-PCR results of the KG candidates showing whole seed or embryo specific or preferable expression pattern [Root_dv: a mixture of roots at 5, 15, 30 days after pollination (DAP); Leaf_dv: a mixture of leaves at 5, 15, 30 DAP; Ear: a mixture of ear at 5 and 10 DAP; whole seeds: a mixture of whole seeds at 15, 20, 30 DAP; Endosperm: a mixture of endosperm at 15, 20, 30 DAP; Embryo: a mixture of embryo at 15, 20, 30 DAP; Root_V2+V4: a mixture of root at V2 and V4 stages; Shoot/leaf_V2+V4: a mixture of V2 shoot and V4 leaves; Flower_GS: a mixture of flower and geminating seeds.]

FIG. 2 (A) and (B) Diagrams of binary KG vectors

FIG. 3: GUS expression in different tissues at different developmental stages driven by p-KG24 in transgenic maize with RHF155

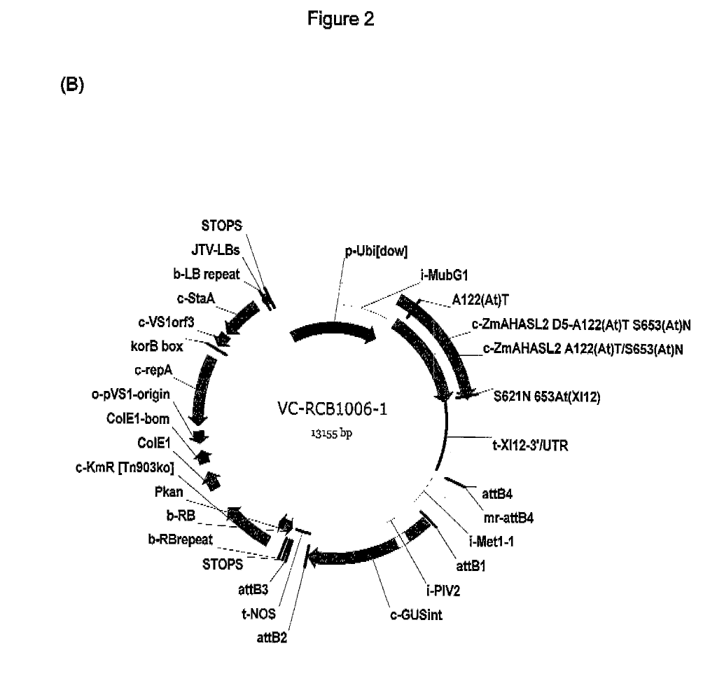

FIG. 4: GUS expression in different tissues at different developmental stages driven by p-KG37 in transgenic maize with RKF109

FIG. 5: GUS expression in different tissues at different developmental stages driven by p-KG45 in transgenic maize with RKF106

FIG. 6: GUS expression in different tissues at different developmental stages driven by p-KG46 in transgenic maize with RKF107

FIG. 7: GUS expression in different tissues at different developmental stages driven by p-KG49 in transgenic maize with RKF108

FIG. 8: GUS expression in different tissues at different developmental stages driven by p-KG56 in transgenic maize with RKF125

FIG. 9: GUS expression in different tissues at different developmental stages driven by p-KG103 in transgenic maize with RHF128

FIG. 10: GUS expression in different tissues at different developmental stages driven by p-KG119 in transgenic maize with RHF138

FIG. 11: GUS expression in different tissues at different developmental stages driven by p-KG129 in transgenic maize with RTP1047

FIG. 12: q-RT-PCR results of the MA candidates [Root_dv: a mixture of roots at 5, 15, 30 days after pollination (DAP); Leaf_dv: a mixture of leaves at 5, 15, 30 DAP; Ear: a mixture of ear at 5 and 10 DAP; whole seeds: a mixture of whole seeds at 15, 20, 30 DAP; Endosperm: a mixture of endosperm at 15, 20, 30 DAP; Embryo: a mixture of embryo at 15, 20, 30 DAP; Root_V2+V4: a mixture of root at V2 and V4 stages; Shoot/leaf_V2+V4: a mixture of V2 shoot and V4 leaves; Flower_GS: a mixture of flower and geminating seeds.]

FIG. 13: Vector RCB 1006 for MAWS promoters

FIG. 14: GUS expression in different tissues at different developmental stages driven by p-MAWS23 in transgenic maize with RTP1060

FIG. 15: GUS expression in different tissues at different developmental stages driven by p-MAWS27 in transgenic maize with RTP1059

FIG. 16: GUS expression in different tissues at different developmental stages driven by p-MAWS30 in transgenic maize with RTP1053

FIG. 17: GUS expression in different tissues at different developmental stages driven by p-MAWS57 in transgenic maize with RTP1049

FIG. 18: GUS expression in different tissues at different developmental stages driven by p-MAWS60 in transgenic maize with RTP1056

FIG. 19: GUS expression in different tissues at different developmental stages driven by p-MAWS63 in transgenic maize with RTP1048

FIG. 20: GUS expression in different tissues at different developmental stages driven by p-MAEM1 in transgenic maize with RTP1061

FIG. 21: GUS expression in different tissues at different developmental stages driven by p-MAEM20 in transgenic maize with RTP1064

FIG. 22: qRT-PCR results of the Zm.8705.1.S1_at

FIG. 23: Digital image of the GenomeWalk (GW) run on a 1% w/v agarose gel and stained with ethidium bromide. The lanes (L) represent as follows: (L1)1 kb plus ladders (Promega, Madison, Wis., USA), (L2) no DNA (replaced GW library with sterile ddH₂O) as negative control; (L3) Human PvuII GW library and primers from Human tissue-type plasminogen activator provided by the kit as a positive control, (L4)B73 PvuII GW library, (L5)B73 EcoRV GW library, (L6)B73 DraI GW library, (7)B73 StuI GW library. L3 using primers from Human tissue-type plasminogen activator (tPA) provided by the kit. L2, and L4 through L7) using ZmNP28-specific primers.

FIG. 24: Final binary vectors RLN 90 (A) and RLN 93 (B); FIG. 24 (C) is a diagram of RHF160 and FIG. 24 (D) is a diagram of RHF158.

FIG. 25: (A) GUS expression in different tissues at different developmental stages driven by pZmNP28_{—}655 in transgenic maize with RLN90; (B) GUS expression in different tissues at different developmental stages driven by pZmNP28_{—}507 in transgenic maize with RLN93; (C) GUS expression in different tissues at different developmental stages driven by pZmNP28_{—}1706 in transgenic maize with RHF158; (D) GUS expression in different tissues at different developmental stages driven by pZmNP28_{—}2070 in transgenic maize with RHF160.

### DESCRIPTION OF THE SEQUENCE IDENTIFICATION NUMBERS REFERRING TO THE PROMOTERS

| | | | amino | | | | | | |
|---|---|---|---|---|---|---|---|---|---|
| Name | Promoter | CDS | acid | vector | Gene | ESTs | Variant 1 | Variant 2 | Fragments |
| MAWS60 | 1 | 19 | 37 | 55 | 73 | 91 | 109 | 127 | |
| MAEM1 | 2 | 20 | 38 | 56 | 74 | 92 | 110 | 128 | |
| KG_56 | 3 | 21 | 39 | 57 | 75 | 93 | 111 | 129 | 145 |
| KG_129 | 4 | 22 | 40 | 58 | 76 | 94 | 112 | 130 | 146 |
| MAEM20 | 5 | 23 | 41 | 59 | 77 | 95 | 113 | 131 | |
| MAWS27 | 6 | 24 | 42 | 60 | 78 | 96 | 114 | 132 | |
| MAWS63 | 7 | 25 | 43 | 61 | 79 | 97 | 115 | 133 | |
| KG_49 | 8 | 26 | 44 | 62 | 80 | 98 | 116 | 134 | 147 |
| KG_24 | 9 | 27 | 45 | 63 | 81 | 99 | 117 | 135 | 148 |
| KG_37 | 10 | 28 | 46 | 64 | 82 | 100 | 118 | 136 | 149 |
| KG_45 | 11 | 29 | 47 | 65 | 83 | 101 | 119 | 137 | 150 |
| KG_46 | 12 | 30 | 48 | 66 | 84 | 102 | 120 | 138 | 151 |
| KG_103 | 13 | 31 | 49 | 67 | 85 | 103 | 121 | 139 | 152 |
| KG_119 | 14 | 32 | 50 | 68 | 86 | 104 | 122 | 140 | 153 |
| MAWS23 | 15 | 33 | 51 | 69 | 87 | 105 | 123 | 141 | |
| MAWS30 | 16 | 34 | 52 | 70 | 88 | 106 | 124 | 142 | |
| MAWS57 | 17 | 35 | 53 | 71 | 89 | 107 | 125 | 143 | |
| ZmNP28 | 18 | 36 | 54 | 72 | 90 | 108 | 126 | 144 | |

### GENERAL DEFINITIONS

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower).

As used herein, the word “or” means any one member of a particular list and also includes any combination of members of that list.

“Expression cassette” as used herein means a linear or circular nucleic acid molecule. It encompasses DNA as well as RNA sequences which are capable of directing expression of a particular nucleotide sequence in an appropriate host cell. In general, it comprises a promoter operably linked to a polynucleotide of interest, which is—optionally—operably linked to termination signals and/or other regulatory elements. The expression cassette of the present invention is characterized in that it shall comprise a transcription regulating nucleotide sequence as defined hereinafter. An expression cassette may also comprise sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the polynucleotide sequence of interest may be chimeric, meaning that at least one of its components is heterologous with respect to at least one of its other components. The expression cassette may also be one, which is naturally occurring but has been obtained in a recombinant form useful for heterologous expression. An expression cassette may be assembled entirely extracellularly (e.g., by recombinant cloning techniques). However, an expression cassette may also be assembled using in part endogenous components. For example, an expression cassette may be obtained by placing (or inserting) a promoter sequence upstream of an endogenous sequence, which thereby becomes functionally linked and controlled by said promoter sequences. Likewise, a nucleic acid sequence to be expressed may be placed (or inserted) downstream of an endogenous promoter sequence thereby forming an expression cassette. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development (e.g., the embryo preferential or embryo specific promoters of the invention). In a preferred embodiment, such expression cassettes will comprise the transcriptional initiation region of the invention linked to a nucleotide sequence of interest. Such an expression cassette is preferably provided with a plurality of restriction sites for insertion of the gene of interest to be under the transcriptional regulation of the regulatory regions. The expression cassette may additionally contain selectable marker genes. The cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions and others described below (see also, Guerineau 1991; Proudfoot 1991; Sanfacon 1991; Mogen 1990; Munroe 1990; Ballas 1989; Joshi 1987). The expression cassette can also comprise a multiple cloning site. In such a case, the multiple cloning site is, preferably, arranged in a manner as to allow for operative linkage of a polynucleotide to be introduced in the multiple cloning site with the transcription regulating sequence. In addition to the aforementioned components, the expression cassette of the present invention, preferably, could comprise components required for homologous recombination, i.e. flanking genomic sequences from a target locus. However, also contemplated is an expression cassette which essentially consists of the transcription regulating nucleotide sequence, as defined hereinafter.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments. A promoter may also contain DNA sequences that are involved in the binding of protein factors, which control the effectiveness of transcription initiation in response to physiological or developmental conditions. The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative. Promoter elements, such as a TATA element, that are inactive or have greatly reduced promoter activity in the absence of upstream activation are referred as “minimal” or “core” promoters. In the presence of a suitable transcription factor, the minimal promoter functions to permit transcription. A “minimal” or “core’ promoter thus consists only of all basal elements needed for transcription initiation, e.g., a TATA box and/or an initiator.

“Constitutive promoter” refers to a promoter that is able to express the open reading frame (ORF) in all or nearly all of the plant tissues during all or nearly all developmental stages of the plant. Each of the transcription-activating elements do not exhibit an absolute tissue-specificity, but mediate transcriptional activation in most plant tissues at a level of at least 1% reached in the plant tissue in which transcription is most active. “Constitutive expression” refers to expression using a constitutive promoter.

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysone-inducible systems. “Conditional” and “regulated expression” refer to expression controlled by a regulated promoter.

“Inducible promoter” refers to those regulated promoters that can be turned on in one or more cell types by an external stimulus, such as a chemical, light, hormone, stress, or a pathogen.

As used herein, “transcription regulating nucleotide sequence”, refers to nucleotide sequences influencing the transcription, RNA processing or stability, or translation of the associated (or functionally linked) nucleotide sequence to be transcribed. The transcription regulating nucleotide sequence may have various localizations with the respect to the nucleotide sequences to be transcribed. The transcription regulating nucleotide sequence may be located upstream (5′ non-coding sequences), within, or downstream (3′ non-coding sequences) of the sequence to be transcribed (e.g., a coding sequence). The transcription regulating nucleotide sequences may be selected from the group comprising enhancers, promoters, translation leader sequences, introns, 5′-untranslated sequences, 3′-untranslated sequences, and polyadenylation signal sequences. They include natural and synthetic sequences as well as sequences, which may be a combination of synthetic and natural sequences. As is noted above, the term “transcription regulating nucleotide sequence” is not limited to promoters. However, preferably a transcription regulating nucleotide sequence of the invention comprises at least one promoter sequence (e.g., a sequence localized upstream of the transcription start of a gene capable to induce transcription of the downstream sequences). In one preferred embodiment the transcription regulating nucleotide sequence of the invention comprises the promoter sequence of the corresponding gene and—optionally and preferably—the native 5′-untranslated region of said gene. Furthermore, the 3′-untranslated region and/or the polyadenylation region of said gene may also be employed.

As used herein, the term “cis-regulatory element” or “promoter motif” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact in different affinities with more than one cis-element. The promoters of the present invention desirably contain cis-elements that can confer or modulate gene expression. Cis-elements can be identified by a number of techniques, including deletion analysis, i.e., deleting one or more nucleotides from the 5′ end or internal of a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis with known cis-element motifs by conventional DNA sequence comparison methods. The fine structure of a cis-element can be further studied by mutagenesis (or substitution) of one or more nucleotides or by other conventional methods. Cis-elements can be obtained by chemical synthesis or by isolation from promoters that include such elements, and they can be synthesized with additional flanking nucleotides that contain useful restriction enzyme sites to facilitate subsequence manipulation.

The “expression pattern” of a promoter (with or without enhancer) is the pattern of expression levels, which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates. The rate of degradation can however be considered to proceed at a fixed rate when the transcribed sequences are identical, and thus this value can serve as a measure of synthesis rates. When promoters are compared in this way, techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA. The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (initiator) plays an important role in determining the level of RNA expression directed by that specific promoter. This includes also part of the transcribed sequences. The direct fusion of promoter to reporter sequences would therefore lead to suboptimal levels of transcription. A commonly used procedure to analyze expression patterns and levels is through determination of the ‘steady state’ level of protein accumulation in a cell. Commonly used candidates for the reporter gene, known to those skilled in the art are beta-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression. Generally, individual transformed lines with one chimeric promoter reporter construct may vary in their levels of expression of the reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence. For the purposes of the present invention, “tissue-specific” preferably refers to “seed-specific” or “seed-preferential” or embryo-specific or embryo-preferential.

“Seed” as used herein refers, preferably, to whole seed, endosperm and embryonic tissues, more preferably to embryonic tissue. “Specific” in the sense of the invention means that the polynucleotide of interest being operatively linked to the transcription regulating nucleotide sequence referred to herein will be predominantly expressed in the indicated tissues or cells when present in a plant. A predominant expression as meant herein is characterized by a statistically significantly higher amount of detectable transcription in the said tissue or cells with respect to other plant tissues. A statistically significant higher amount of transcription is, preferably, an amount being at least two-fold, three-fold, four-fold, five-fold, ten-fold, hundred-fold, five hundred-fold or thousand-fold the amount found in at least one of the other tissues with detectable transcription. Alternatively, it is an expression in the indicated tissue or cell whereby the amount of transcription in other tissues or cells is less than 1%, 2%, 3%, 4% or, most preferably, 5% of the overall (whole plant) amount of expression. The amount of transcription directly correlates to the amount of transcripts (i.e. RNA) or polypeptides encoded by the transcripts present in a cell or tissue. Suitable techniques for measuring transcription either based on RNA or polypeptides are well known in the art. Tissue or cell specificity alternatively and, preferably in addition to the above, means that the expression is restricted or almost restricted to the indicated tissue or cells, i.e. there is essentially no detectable transcription in other tissues. Almost restricted as meant herein means that unspecific expression is detectable in less than ten, less than five, less than four, less than three, less than two or one other tissue(s). “Seed-preferential” or “embryo-preferential” in the context of this invention means the transcription of a nucleic acid sequence by a transcription regulating element in a way that transcription of said nucleic acid sequence in seeds contribute to more than 50%, preferably more than 70%, more preferably more than 80% of the entire quantity of the RNA transcribed from said nucleic acid sequence in the entire plant during any of its developmental stage.

“Expression” refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

Seed specific expression can be determined by comparing the expression of a nucleic acid of interest, e.g., a reporter gene such as GUS, operatively linked to the expression control sequence in the following tissues and stages: 1) roots and leafs at 5-leaf stage, 2) stem at V-7 stage, 3) Leaves, husk, and silk at flowering stage at the first emergence of silk, 4) Spikelets/Tassel at pollination, 5) Ear or Kernels at 5, 10, 15, 20, and 25 days after pollination. Preferably, expression of the nucleic acid of interest can be determined only in Ear or Kernels at 5, 10, 15, 20, and 25 days after pollination in said assay as shown in the accompanying Figures. The expression of the polynucleotide of interest can be determined by various well known techniques, e.g., by Northern Blot or in situ hybridization techniques as described in WO 02/102970, and, preferably, by GUS histochemical analysis as described in the accompanying Examples. Transgenic plants for analyzing seed specific expression can be also generated by techniques well known to the person skilled in the art and as discussed elsewhere in this specification.

The term “nucleic acid” refers to deoxyribonucleotides or ribonucleotides and their polymers thereof in either single- or double-stranded form, composed of monomers (nucleotides) containing a sugar, phosphate and a base, which is either a purine or pyrimidine. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer 1991; Ohtsuka 1985; Rossolini 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals. The nucleotide sequences of the invention include both the naturally occurring sequences as well as mutant (variant) forms. Such variants will continue to possess the desired activity, i.e., either promoter activity or the activity of the product encoded by the open reading frame of the non-variant nucleotide sequence.

The term “variant” with respect to a sequence (e.g., a polypeptide or nucleic acid sequence such as—for example—a transcription regulating nucleotide sequence of the invention) is intended to mean substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence, i.e. for example to SEQ ID NO's:1 to 18 or 19 to 36.

The nucleic acid molecules of the invention can be “optimized” for enhanced expression in plants of interest (see, for example, WO 91/16432; Perlak 1991; Murray 1989). In this manner, the open reading frames in genes or gene fragments can be synthesized utilizing plant-preferred codons (see, for example, Campbell & Gowri, 1990 for a discussion of host-preferred codon usage). Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art (see, for example, Stemmer 1994; Stemmer 1994; Crameri 1997; Moore 1997; Zhang 1997; Crameri 1998; and U.S. Pat. Nos. 5,605,794, 6, 8, 10, and 12,837,458).

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.
(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.
(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.
   Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, 1988; the local homology algorithm of Smith et al. 1981; the homology alignment algorithm of Needleman and Wunsch 1970; the search-for-similarity-method of Pearson and Lipman 1988; the algorithm of Karlin and Altschul, 1990, modified as in Karlin and Altschul, 1993.
   Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described (Higgins 1988, 1989; Corpet 1988; Huang 1992; Pearson 1994). The ALIGN program is based on the algorithm of Myers and Miller, supra. The BLAST programs of Altschul et al., 1990, are based on the algorithm of Karlin and Altschul, supra. Multiple aligments (i.e. of more than 2 sequences) are preferably performed using the Clustal W algorithm (Thompson 1994; e.g., in the software Vector NTI™, version 9; Invitrogen Inc.) with the scoring matrix BLOSUM62MT2 with the default settings (gap opening penalty 15/19, gap extension penalty 6.66/0.05; gap separation penalty range 8; % identity for alignment delay 40; using residue specific gaps and hydrophilic residue gaps).
   Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.
   In addition to calculating percent sequence identity, the BLAST algorithm also performs a statistical analysis of the similarity between two sequences (see, e.g., Karlin & Altschul (1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.
   To obtain gapped alignments for comparison purposes, Gapped BLAST (in BLAST 2.0) can be utilized as described in Altschul et al. 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.
   For purposes of the present invention, comparison of nucleotide sequences for determination of percent sequence identity to specific nucleotide sequences (e.g., the promoter sequences disclosed herein) is preferably made using the BlastN program (version 1.4.7 or later) with its default parameters (wordlength (W) of 11, an expectation (E) of 10, a cutoff of 100, M=5, N=−4, and a comparison of both strands) or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.
   For purposes of the present invention, comparison of polypeptide or amino acid sequences for determination of percent sequence identity/homology to specific polypeptide or amino acid sequences is preferably made using the BlastP program (version 1.4.7 or later) with its default parameters (wordlength (W) of 3, an expectation (E) of 10, and the BLOSUM62 scoring matrix (Henikoff & Henikoff, 1989); see http://www.ncbi.nlm.nih.gov) or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.
   (c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).
   (d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.
   (e) (i) The term “substantial identity” of polynucleotide sequences means that a polynucleotide comprises a sequence that has at least 38%, e.g., 39%, 40%, 42%, 44%, 46%, 48%, 50%, 52%, 54%, 56%, 58%, 60%, 62%, 64%, 65%, 66%, 67%, 68%, 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 38%, 50% or 60%, preferably at least 70% or 80%, more preferably at least 90%, 95%, and most preferably at least 98%.
   Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.
   (ii) The term “substantial identity” in the context of a peptide indicates that a peptide comprises a sequence with at least 38%, e.g. 39%, 40%, 42%, 44%, 46%, 48%, 50%, 52%, 54%, 56%, 58%, 60%, 62%, 64%, 65%, 66%, 67%, 68%, 69%, 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984:

Tₘ=81.5° C.+16.6(log₁₀ M)+0.41(% GC)−0.61(% form)−500/L

where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4 to 6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of highly stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C. (very low stringency conditions), more desirably in 7% sodium dodecyl sulfate (SDS), 0.5 M NaPO₄, 1 mM EDTA at 50° C. with washing in 1×SSC, 0.1% SDS at 50° C. (low stringency conditions), more desirably still in 7% sodium dodecyl sulfate (SDS), 0.5 M NaPO₄, 1 mM EDTA at 50° C. with washing in 0.5×SSC, 0.1% SDS at 50° C. (moderate stringency conditions), preferably in 7% sodium dodecyl sulfate (SDS), 0.5 M NaPO₄, 1 mM EDTA at 50° C. with washing in 0.1×SSC, 0.1% SDS at 50° C. (high stringency conditions), more preferably in 7% sodium dodecyl sulfate (SDS), 0.5 M NaPO₄, 1 mM EDTA at 50° C. with washing in 0.1×SSC, 0.1% SDS at 65° C. (very high stringency conditions).

The terms “open reading frame” and “ORF” refer to the amino acid sequence encoded between translation initiation and termination codons of a coding sequence. The terms “initiation codon” and “termination codon” refer to a unit of three adjacent nucleotides ('codon') in a coding sequence that specifies initiation and chain termination, respectively, of protein synthesis (mRNA translation).

“Encoding” or “Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

“Operably-linked” or “functionally linked” refers preferably to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide sequence identity refers to the similarity between the nucleotide sequences of two nucleic acid molecules or between the amino acid sequences of two protein molecules. Estimates of such homology are provided by either DNA-DNA or DNA-RNA hybridization under conditions of stringency as is well understood by those skilled in the art (as described in Haines and Higgins (eds.), Nucleic Acid Hybridization, IRL Press, Oxford, U.K.), or by the comparison of sequence similarity between two nucleic acids or proteins.

“Vector” is defined to include, inter alia, any plasmid, cosmid, phage or Agrobacterium binary nucleic acid molecule in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance, kanamycin resistance, streptomycin resistance or ampicillin resistance.

A “transgene” or “trangenic” refers to a gene that has been introduced into the genome by transformation and is stably or transiently maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

The term “transformation” refers to the transfer of a nucleic acid fragment into the genome of a host cell. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere 1987) and particle bombardment technology (U.S. Pat. No. 4,945,050). Whole plants may be regenerated from transgenic cells by methods well known to the skilled artisan (see, for example, Fromm 1990).

“Transformed,” “transgenic and “recombinant” refer to a host organism such as a bacterium or a plant into which a heterologous nucleic acid molecule has been introduced. The nucleic acid molecule can be stably integrated into the genome generally known in the art and are disclosed (Sambrook 1989; Innis 1995; Gelfand 1995; Innis & Gelfand 1999). For example, “transformed,” “transformant,” and “transgenic” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Chromosomally-integrated” refers to the integration of a foreign gene or DNA construct into the host genome by covalent bonds. Where genes are not “chromosomally integrated”, they may be “transiently expressed”. Transient expression of a gene refers to the expression of a gene that is not integrated into the host chromosome but functions independently, either as part of an autonomously replicating plasmid or expression cassette, for example, or as part of another biological system such as a virus. “Genetically stable” and “heritable” refer to chromosomally-integrated genetic elements that are stably maintained in the plant and stably inherited by progeny through successive generations.

A “transgenic plant” is a plant having one or more plant cells that contain an expression vector as defined hereinafter in the detailed description.

“Primary transformant” and “TO generation” refer to transgenic plants that are of the same genetic generation as the tissue which was initially transformed (i.e., not having gone through meiosis and fertilization since transformation).

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The term “altered plant trait” means any phenotypic or genotypic change in a transgenic plant relative to the wild-type or non-transgenic plant host.

The word “plant” refers to any plant, particularly to agronomically useful plants (e.g., seed plants), and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ differentiated into a structure that is present at any stage of a plant's development. Such structures include one or more plant organs including, but are not limited to, fruit, shoot, stem, leaf, flower petal, etc. Preferably, the term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous. Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom.

### DETAILED DESCRIPTION OF THE INVENTION

The present invention thus provides isolated nucleic acid molecules comprising a plant nucleotide sequence that directs seed-preferential or seed-specific transcription of an operably linked nucleic acid fragment in a plant cell.

Specifically, the present invention provides an expression cassette for regulating seed-specific expression of a polynucleotide of interest, said expression cassette comprising a transcription regulating nucleotide sequence selected from the group of sequences consisting of:
(a) a nucleic acid sequence of SEQ ID NO: 1, 2, 3, 4, 5, 6, 7, 8, 9, 10, 11, 12, 13, 14, 15, 16, 17, or 18, or a variant thereof.
(b) a nucleic acid sequence which is at least 80% identical to a nucleic acid sequence shown in any one of SEQ ID NO: 1, 2, 3, 4, 5, 6, 7, 8, 9, 10, 11, 12, 13, 14, 15, 16, 17, or 18;
(c) a nucleic acid sequence which hybridizes under stringent conditions to a nucleic acid sequence of SEQ ID NO: 1, 2, 3, 4, 5, 6, 7, 8, 9, 10, 11, 12, 13, 14, 15, 16, 17, or 18;
(d) a nucleic acid sequence which hybridizes to a nucleic acid sequence located upstream of an open reading frame sequence of SEQ ID NOs: 19, 20, 21, 22, 23, 24, 25, 26, 27, 28, 29, 30, 31, 32, 33, 34, 35 or 36;
(e) a nucleic acid sequence which hybridizes to a nucleic acid sequence located upstream of an open reading frame sequence encoding an amino acid sequence of SEQ ID NOs: 37, 38, 39, 40, 41, 42, 43, 44, 45, 46, 47, 48, 49, 50, 51, 52, 53 or 54;
(f) a nucleic acid sequence which hybridizes to a nucleic acid sequence located upstream of an open reading frame sequence being at least 80% identical to an open reading frame sequence of SEQ ID NOs: 19, 20, 21, 22, 23, 24, 25, 26, 27, 28, 29, 30, 31, 32, 33, 34, 35 or 36, wherein the open reading frame encodes a seed protein;
(g) a nucleic acid sequence which hybridizes to a nucleic acid sequences located upstream of an open reading frame encoding an amino acid sequence being at least 80% identical to an amino acid sequence as shown in SEQ ID NOs: 37, 38, 39, 40, 41, 42, 43, 44, 45, 46, 47, 48, 49, 50, 51, 52, 53 or 54, wherein the open reading frame encodes a seed protein;
(h) a nucleic acid sequence obtainable by 5′ genome walking or by thermal asymmetric interlaced polymerase chain reaction (TAIL-PCR) on genomic DNA from the first exon of an open reading frame sequence as shown in SEQ ID NOs: 19, 20, 21, 22, 23, 24, 25, 26, 27, 28, 29, 30, 31, 32, 33, 34, 35 or 36; and
(i) a nucleic acid sequence obtainable by 5′ genome walking or TAIL PCR on genomic DNA from the first exon of an open reading frame sequence being at least 80% identical to an open reading frame as shown in SEQ ID NOs: 19, 20, 21, 22, 23, 24, 25, 26, 27, 28, 29, 30, 31, 32, 33, 34, 35 or 36, wherein the open reading frame encodes a seed protein; and
(j) a nucleic acid sequence obtainable by 5′ genome walking or TAIL PCR on genomic DNA from the first exon of an open reading frame sequence encoding an amino acid sequence being at least 80% identical to an amino acid sequence encoded by an open reading frame as shown in SEQ ID NOs: 37, 38, 39, 40, 41, 42, 43, 44, 45, 46, 47, 48, 49, 50, 51, 52, 53 or 54, wherein the open reading frame encodes a seed protein.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1507, 125 to about 1507, 250 to about 1507, 400 to about 1507, 600 to about 1507, upstream of the ATG (1610-1612) located at position 106 to 1612 of SEQ ID NO: 81, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1507, 125 to about 1507, 250 to about 1507, 400 to about 1507, 600 to about 1507, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1507, 125 to about 1507, 250 to about 1507, 400 to about 1507, 600 to about 1507, upstream of the ATG located at position 1610 to 1612 of SEQ ID NO: 81, which include the minimal promoter region. The above-defined stretch of contiguous nucleotides preferably comprises one or more promoter motifs, as shown in Table 22, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 9, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 910, 125 to about 910, 250 to about 910, 400 to about 910, 600 to about 910, upstream of the ATG (1748-1750) located at position 825 to 1735 of SEQ ID NO: 82, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 910, 125 to about 910, 250 to about 910, 400 to about 910, 600 to about 910, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 910, 125 to about 910, 250 to about 910, 400 to about 910, 600 to about 910, upstream of the ATG (1748-1750) located at position 825 to 1735 of SEQ ID NO: 82, which include the minimal promoter region. The above-defined stretch of contiguous nucleotides preferably comprises one or more promoter motifs, as shown in Table 23, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 10, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1131, 125 to about 1131, 250 to about 1131, 400 to about 1131, 600 to about 1131, upstream of the ATG (1185-1160) located at position 44 to 1174 of SEQ ID NO: 83, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1131, 125 to about 1131, 250 to about 1131, 400 to about 1131, 600 to about 1131, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1131, 125 to about 1131, 250 to about 1131, 400 to about 1131, 600 to about 1131, upstream of the ATG (1185-1160) located at position 44 to 1174 of SEQ ID NO: 83, which include the minimal promoter region. The above-defined stretch of contiguous nucleotides preferably comprises one or more promoter motifs, as shown in Table 24, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 11, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 563, 125 to about 563, 250 to about 563, 400 to about 563, upstream of the ATG (624-626) located at position 52 to 614 of SEQ ID NO: 84, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 563, 125 to about 563, 250 to about 563, 400 to about 563, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 563, 125 to about 563, 250 to about 563, 400 to about 563, upstream of the ATG (624-626) located at position 52 to 614 of SEQ ID NO: 84, which include the minimal promoter region. The above-defined stretch of contiguous nucleotides preferably comprises one or more promoter motifs, as shown in Table 25, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 12, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1188, 125 to about 1188, 250 to about 1188, 400 to about 1188, 600 to about 1188, upstream of the ATG (1234-1236) located at position 46 to 1233 of SEQ ID NO: 80, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1188, 125 to about 1188, 250 to about 1188, 400 to about 1188, 600 to about 1188, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1188, 125 to about 1188, 250 to about 1188, 400 to about 1188, 600 to about 1188, upstream of the ATG (1234-1236) located at position 46 to 1233 of SEQ ID NO: 80, which include the minimal promoter region. The above-defined stretch of contiguous nucleotides preferably comprises one or more promoter motifs, as shown in Table 26, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 8, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1945, 125 to about 1945, 250 to about 1945, 400 to about 1945, 600 to about 1945, upstream of the ATG (2428 to 2430) located at position 435 to 2379 of SEQ ID NO: 75, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1945, 125 to about 1945, 250 to about 1945, 400 to about 1945, 600 to about 1945, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1945, 125 to about 1945, 250 to about 1945, 400 to about 1945, 600 to about 1945, upstream of the ATG (2428 to 2430) located at position 435 to 2379 of SEQ ID NO: 75, which include the minimal promoter region. The above-defined stretch of contiguous nucleotides preferably comprises one or more promoter motifs, as shown in Table 27, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 3, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 991, 125 to about 991, 250 to about 991, 400 to about 991, 600 to about 991, upstream of the ATG (996 to 998) located at position 4 to 994 of SEQ ID NO: 85, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 991, 125 to about 991, 250 to about 991, 400 to about 991, 600 to about 991, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 991, 125 to about 991, 250 to about 991, 400 to about 991, 600 to about 991, upstream of the ATG (996 to 998) located at position 4 to 994 of SEQ ID NO: 85, which include the minimal promoter region. The above-defined stretch of contiguous nucleotides preferably comprises one or more promoter motifs, as shown in Table 28, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 13, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 2519, 125 to about 2519, 250 to about 2519, 400 to about 2519, 600 to about 2519, 5 base pairs downstream of the ATG (2511 to 2513) located at position 1 to 2519 of SEQ ID NO: 86, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 2519, 125 to about 2519, 250 to about 2519, 400 to about 2519, 600 to about 2519, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 2519, 125 to about 2519, 250 to about 2519, 400 to about 2519, 600 to about 2519, upstream of the ATG (2511 to 2513) located at position 1 to 2519 of SEQ ID NO: 86, which include the minimal promoter region. The above-defined stretch of contiguous nucleotides preferably comprises one or more promoter motifs, as shown in Table 29, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 14, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 512, 125 to about 512, 250 to about 512, 400 to about 512, upstream of the ATG (678 to 680) located at position 47 to 558 of SEQ ID NO: 76, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 512, 125 to about 512, 250 to about 512, 400 to about 512, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 512, 125 to about 512, 250 to about 512, 400 to about 512, 600 to about 512, upstream of the ATG (678 to 680) located at position 47 to 558 of SEQ ID NO: 76, which include the minimal promoter region. The above-defined stretch of contiguous nucleotides preferably comprises one or more promoter motifs, as shown in Table 30, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 4, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1264, 125 to about 1264, 250 to about 1264, 400 to about 1264, 600 to about 1264, upstream of the ATG (1341 to 1343) located at position 1 to 1264 of SEQ ID NO: 87, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1264, 125 to about 1264, 250 to about 1264, 400 to about 1264, 600 to about 1264, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1264, 125 to about 1264, 250 to about 1264, 400 to about 1264, 600 to about 1264, upstream of the ATG (1341 to 1343) located at position 1 to 1264 of SEQ ID NO: 87, which include the minimal promoter region. The above-defined stretch of contiguous nucleotides preferably comprises one or more promoter motifs, as shown in Table 49, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 15, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1355, 125 to about 1355, 250 to about 1355, 400 to about 1355, 600 to about 1355, upstream of the ATG (1357 to 1359) located at position 1 to 1355 of SEQ ID NO: 78, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1355, 125 to about 1355, 250 to about 1355, 400 to about 1355, 600 to about 1355, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1355, 125 to about 1355, 250 to about 1355, 400 to about 1355, 600 to about 1355, upstream of the ATG (1357 to 1359) located at position 1 to 1355 of SEQ ID NO: 78, which include the minimal promoter region. The above-defined stretch of contiguous nucleotides preferably comprises one or more promoter motifs, as shown in Table 50, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 6, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 623, 125 to about 623, 250 to about 623, 400 to about 623, 500 to about 623, upstream of the ATG (695 to 697) located at position 1 to 623 of SEQ ID NO: 88, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 623, 125 to about 623, 250 to about 623, 400 to about 623, 500 to about 623, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 623, 125 to about 623, 250 to about 623, 400 to about 623, 500 to about 1355, upstream of the ATG (695 to 697) located at position 1 to 623 of SEQ ID NO: 88, which include the minimal promoter region. The above-defined stretch of contiguous nucleotides preferably comprises one or more promoter motifs, as shown in Table 51, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 16, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1950, 125 to about 1950, 250 to about 1950, 400 to about 1950, 600 to about 1950, upstream of the ATG (2700 to 2702) located at position 700 to 2649 of SEQ ID NO: 89, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1950, 125 to about 1950, 250 to about 1950, 400 to about 1950, 600 to about 1950, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1950, 125 to about 1950, 250 to about 1950, 400 to about 1950, 600 to about 1355, upstream of the ATG (2700 to 2702) located at position 700 to 2649 of SEQ ID NO: 89, which include the minimal promoter region. The above-defined stretch of contiguous nucleotides preferably comprises one or more promoter motifs, as shown in Table 52, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 17, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1106, 125 to about 1106, 250 to about 1106, 400 to about 1106, 600 to about 1106, upstream of the ATG (1220 to 1222) located at position 1 to 1106 of SEQ ID NO: 73, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1106, 125 to about 1106, 250 to about 1106, 400 to about 1106, 600 to about 1106, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1106, 125 to about 1106, 250 to about 1106, 400 to about 1106, 600 to about 1355, upstream of the ATG (1220 to 1222) located at position 1 to 1106 of SEQ ID NO: 73, which include the minimal promoter region. The above-defined stretch of contiguous nucleotides preferably comprises one or more promoter motifs, as shown in Table 53, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 1, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 1941, 125 to about 1941, 250 to about 1941, 400 to about 1941, 600 to about 1941, upstream of the ATG (2303 to 2305) located at position 302 to 2242 of SEQ ID NO: 79, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 1941, 125 to about 1941, 250 to about 1941, 400 to about 1941, 600 to about 1941, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 1941, 125 to about 1941, 250 to about 1941, 400 to about 1941, 600 to about 1355, upstream of the ATG (2303 to 2305) located at position 302 to 2242 of SEQ ID NO: 79, which include the minimal promoter region. The above-defined stretch of contiguous nucleotides preferably comprises one or more promoter motifs, as shown in Table 54, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 7, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 922, 125 to about 922, 250 to about 922, 400 to about 922, 600 to about 922, upstream of the ATG (923 to 925) located at position 1 to 922 of SEQ ID NO: 74, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 922, 125 to about 922, 250 to about 922, 400 to about 922, 600 to about 922, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 922, 125 to about 922, 250 to about 922, 400 to about 922, 600 to about 1355, upstream of the ATG (923 to 925) located at position 1 to 922 of SEQ ID NO: 74, which include the minimal promoter region. The above-defined stretch of contiguous nucleotides preferably comprises one or more promoter motifs, as shown in Table 55, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 2, or a variant thereof.

Preferably, the transcription regulating nucleotide sequence and promoters of the invention include a consecutive stretch of about 25 to 3000, including 50 to 2000 or 100 to 500, and up to 1000 or 1500, contiguous nucleotides, e.g., 40 to about 2500, 60 to about 698, 125 to about 698, 250 to about 698, 400 to about 698, 500 to about 698, upstream of the ATG (699 to 671) located at position 1 to 698 of SEQ ID NO: 77, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 698, 125 to about 698, 250 to about 698, 400 to about 698, 500 to about 698, has at least 50% or 60%, preferably at least 70% or 80%, more preferably at least 90% and most preferably at least 95%, nucleic acid sequence identity with a corresponding consecutive stretch of about 25 to 3000, including 50 to 2000 or 100 to 500, and up to 1000 or 1500, contiguous nucleotides, e.g., 40 to about 2500, 60 to about 698, 125 to about 698, 250 to about 698, 400 to about 698, 500 to about 1355, upstream of the ATG (699 to 671) located at position 1 to 698 of SEQ ID NO: 77, which include the minimal promoter region. The above-defined stretch of contiguous nucleotides preferably comprises one or more promoter motifs, as shown in Table 56, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 5, or a variant thereof.

Preferably, the transcription regulating nucleotide sequence and promoters of the invention include a consecutive stretch of about 25 to 3500, including 50 to 3000 or 100 to 500, and up to 1000 or 1500, contiguous nucleotides, e.g., 40 to about 3500, 60 to about 3000, 125 to about 2500, 250 to about 2300, 400 to about 2000, 600 to about 1700, upstream of the ATG located at position 656 to 658 of SEQ ID NO: 196, which include the minimal promoter region.

In a particular embodiment of the invention said consecutive stretch of about 25 to 3000, including 50 to 2000 or 100 to 500, and up to 1000 or 1500, contiguous nucleotides, e.g., 40 to about 2500, 60 to about 922, 125 to about 922, 250 to about 922, 400 to about 922, 600 to about 922, has at least 50% or 60%, preferably at least 70% or 80%, more preferably at least 90% and most preferably at least 95%, nucleic acid sequence identity with a corresponding consecutive stretch of about 25 to 3500, including 50 to 3000 or 100 to 500, and up to 1000 or 1500, contiguous nucleotides, e.g., 40 to about 3500, 60 to about 3000, 125 to about 2500, 250 to about 2300, 400 to about 2000, 600 to about 1700, upstream of the ATG located at position 656 to 658 of SEQ ID NO: 196, which include the minimal promoter region. The above-defined stretch of contiguous nucleotides preferably comprises one or more promoter motifs, as shown in Table 61, preferably selected from the group consisting of TATA box, GC-box, CAAT-box and a transcription start site. A preferred transcription regulating nucleotide sequence to be included into an expression cassette of the present invention has a nucleic acid sequence as shown in SEQ ID NO: 18, or a variant thereof.

In a particularly preferred embodiment said consecutive stretch of nucleotides comprises nucleotide 1440 to 2112 of SEQ ID NO: 18, nucleotide 1600 to 2112 of SEQ ID NO: 18, even more preferred nucleotide 1740 to 2112 of SEQ ID NO: 18, and most preferred nucleotide 1740 to 1999 of SEQ ID NO: 18.

The present invention also contemplates a transcription regulating nucleotide sequences which can be derived from a transcription regulating nucleotide sequence shown in SEQ ID NO: 1, 2, 3, 4, 5, 6, 7, 8, 9, 10, 11, 12, 13, 14, 15, 16, 17, or 18. Said transcription regulating nucleotide sequences are capable of hybridizing, preferably under stringent conditions, to the upstream sequences of the open reading frame shown in SEQ ID NO: 19, 20, 21, 22, 23, 24, 25, 26, 27, 28, 29, 30, 31, 32, 33, 34, 35 or 36, or a variant thereof, i.e. to the transcription regulating nucleotide sequences shown in SEQ ID NOs: 1, 2, 3, 4, 5, 6, 7, 8, 9, 10, 11, 12, 13, 14, 15, 16, 17, or 18, or a variant thereof.

Stringent hybridization conditions as meant herein are, preferably, hybridization conditions in 6× sodium chloride/sodium citrate (═SSC) at approximately 45° C., followed by one or more wash steps in 0.2×SSC, 0.1% SDS at 53 to 65° C., preferably at 55° C., 56° C., 57° C., 58° C., 59° C., 60° C., 61° C., 62° C., 63° C., 64° C. or 65° C. The skilled worker knows that these hybridization conditions differ depending on the type of nucleic acid and, for example when organic solvents are present, with regard to the temperature and concentration of the buffer. Examples for stringent hybridization conditions are given in the “General Definitions” section.

Moreover, transcription regulating nucleotide sequences of the present invention can not only be found upstream of the aforementioned open reading frames having a nucleic acid sequence as shown in SEQ ID NO: 19, 20, 21, 22, 23, 24, 25, 26, 27, 28, 29, 30, 31, 32, 33, 34, 35 or 36. Rather, transcription regulating nucleotide sequences can also be found upstream of orthologous, paralogous or homologous genes (i.e. open reading frames). Thus, also preferably, a variant transcription regulating nucleotide sequence comprised by an expression cassette of the present invention has a nucleic acid sequence which hybridizes to a nucleic acid sequences located upstream of an open reading frame sequence being at least 70%, more preferably, at least 80%, at least 90%, at least 91%, at least 92%, at least 93%, at least 94% at least 95%, at least 96%, at least 97%, at least 98%, or at least 99% identical to a sequence as shown in SEQ ID NOs: 19, 20, 21, 22, 23, 24, 25, 26, 27, 28, 29, 30, 31, 32, 33, 34, 35 or 36. The said variant open reading shall encode a polypeptide having the biological activity of the corresponding polypeptide being encoded by the open reading frame shown in SEQ ID NOs: 19, 20, 21, 22, 23, 24, 25, 26, 27, 28, 29, 30, 31, 32, 33, 34, 35 or 36. In this context it should be mentioned that the open reading frame shown in SEQ ID NOs: 19, 20, 21, 22, 23, 24, 25, 26, 27, 28, 29, 30, 31, 32, 33, 34, 35 or 36 encodes a polypeptide having the amino acid sequence shown in SEQ ID NOs: 37, 38, 39, 40, 41, 42, 43, 44, 45, 46, 47, 48, 49, 50, 51, 52, 53 or 54 and, preferably, encodes a seed protein.

Also preferably, a variant transcription regulating nucleotide sequence of the present invention is (i) obtainable by 5′ genome walking or TAIL PCR from an open reading frame sequence as shown in SEQ ID NOs: 19, 20, 21, 22, 23, 24, 25, 26, 27, 28, 29, 30, 31, 32, 33, 34, 35 or 36 or (ii) obtainable by 5′ genome walking or TAIL PCR from a open reading frame sequence being at least 80% identical to an open reading frame as shown in SEQ ID NOs: 19, 20, 21, 22, 23, 24, 25, 26, 27, 28, 29, 30, 31, 32, 33, 34, 35 or 36. Variant expression control sequences are obtainable without further ado by the genome walking technology or by thermal asymmetric interlaced polymerase chain reaction (TAIL-PCR) which can be carried out as described by Liu and Huang, Plant Molecular Biology Reporter, 1998, Vol. 16, pages 175 to 181, as well as references therein, or Liu et al., The Plant Journal, 1995, Vol. 8, pages 457-463, and references therein, by using, e.g., commercially available kits.

Suitable oligonucleotides corresponding to a nucleotide sequence of the invention, e.g., for use as primers in probing or amplification reactions as the PCR reaction described abobe, may be about 30 or fewer nucleotides in length (e.g., 9, 12, 15, 18, 20, 21, 22, 23, or 24, or any number between 9 and 30). Generally specific primers are upwards of 14 nucleotides in length. For optimum specificity and cost effectiveness, primers of 16 to 24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length.

Variant transcription regulating nucleotide sequences referred to in this specification for the transcription regulating nucleotide sequence shown in SEQ ID NO: 9, preferably, comprise at least 10, at least 20, at least 30, or all of the sequence motifs recited in Table 22.

Variant transcription regulating nucleotide sequences referred to in this specification for the transcription regulating nucleotide sequence shown in SEQ ID NO: 10, preferably, comprise at least 10, at least 20, at least 30, or all of the sequence motifs recited in Table 23.

Variant transcription regulating nucleotide sequences referred to in this specification for the transcription regulating nucleotide sequence shown in SEQ ID NO: 11, preferably, comprise at least 10, at least 20, at least 30, or all of the sequence motifs recited in Table 24.

Variant transcription regulating nucleotide sequences referred to in this specification for the transcription regulating nucleotide sequence shown in SEQ ID NO: 12, preferably, comprise at least 10, at least 20, at least 30, or all of the sequence motifs recited in Table 25.

Variant transcription regulating nucleotide sequences referred to in this specification for the transcription regulating nucleotide sequence shown in SEQ ID NO: 8, preferably, comprise at least 10, at least 20, at least 30, or all of the sequence motifs recited in Table 26.

Variant transcription regulating nucleotide sequences referred to in this specification for the transcription regulating nucleotide sequence shown in SEQ ID NO: 3, preferably, comprise at least 10, at least 20, at least 30, or all of the sequence motifs recited in Table 27.

Variant transcription regulating nucleotide sequences referred to in this specification for the transcription regulating nucleotide sequence shown in SEQ ID NO: 13, preferably, comprise at least 10, at least 20, at least 30, or all of the sequence motifs recited in Table 28

Variant transcription regulating nucleotide sequences referred to in this specification for the transcription regulating nucleotide sequence shown in SEQ ID NO: 14, preferably, comprise at least 10, at least 20, at least 30, or all of the sequence motifs recited in Table 29.

Variant transcription regulating nucleotide sequences referred to in this specification for the transcription regulating nucleotide sequence shown in SEQ ID NO: 4, preferably, comprise at least 10, at least 20, at least 30, or all of the sequence motifs recited in Table 30.

Variant transcription regulating nucleotide sequences referred to in this specification for the transcription regulating nucleotide sequence shown in SEQ ID NO: 15, preferably, comprise at least 10, at least 20, at least 30, or all of the sequence motifs recited in Table 49.

Variant transcription regulating nucleotide sequences referred to in this specification for the transcription regulating nucleotide sequence shown in SEQ ID NO: 6, preferably, comprise at least 10, at least 20, at least 30, or all of the sequence motifs recited in Table 50.

Variant transcription regulating nucleotide sequences referred to in this specification for the transcription regulating nucleotide sequence shown in SEQ ID NO: 16, preferably, comprise at least 10, at least 20, at least 30, or all of the sequence motifs recited in Table 51.

Variant transcription regulating nucleotide sequences referred to in this specification for the transcription regulating nucleotide sequence shown in SEQ ID NO: 17 preferably, comprise at least 10, at least 20, at least 30, or all of the sequence motifs recited in Table 52.

Variant transcription regulating nucleotide sequences referred to in this specification for the transcription regulating nucleotide sequence shown in SEQ ID NO: 1, preferably, comprise at least 10, at least 20, at least 30, or all of the sequence motifs recited in Table 53.

Variant transcription regulating nucleotide sequences referred to in this specification for the transcription regulating nucleotide sequence shown in SEQ ID NO: 7, preferably, comprise at least 10, at least 20, at least 30, or all of the sequence motifs recited in Table 54.

Variant transcription regulating nucleotide sequences referred to in this specification for the transcription regulating nucleotide sequence shown in SEQ ID NO: 2, preferably, comprise at least 10, at least 20, at least 30, or all of the sequence motifs recited in Table 55.

Variant transcription regulating nucleotide sequences referred to in this specification for the transcription regulating nucleotide sequence shown in SEQ ID NO: 5, preferably, comprise at least 10, at least 20, at least 30, or all of the sequence motifs recited in Table 56.

Variant transcription regulating nucleotide sequences referred to in this specification for the transcription regulating nucleotide sequence shown in SEQ ID NO: 18, preferably, comprise at least 10, at least 20, at least 30, or all of the sequence motifs recited in Table 61.

Examples for preferred variant transcription regulating sequences are shown in SEQ ID NOs 109 to 126 as well as 127 to 144.

Compared to the corresponding transcription regulating nucleotide sequences, the aforementioned variants (as shown in SEQ ID NOs: 109 to 144) do not comprise start codons (ATG). The start codons are either replaced by BVH (SEQ ID NOs: 109, 110, 111, 112, 113, 114, 115, 116, 117, 118, 119, 120, 121, 122, 123, 124, 125, 126) or by BVH plus stop codons (SEQ ID NOs: 127, 128, 129, 130, 131, 132, 133, 134, 135, 136, 137, 138, 139, 140, 141, 142, 143, 144) between any two start codons (according to the IUPAC nomenclature: B represents C or G or T, V represents A or C or G, and H represents A or C or T). Thus, variant transcription regulating sequences may be obtained by mutating putative start codons as described above.

Without significantly impairing the properties mentioned, non-essential sequences of the transcription regulating nucleotide sequence of the invention can be deleted. Delimitation of the expression control sequence to particular essential regulatory regions can also be undertaken with the aid of a computer program such as the PLACE program (“Plant Cis-acting Regulatory DNA Elements”) (Higo K et al. (1999) Nucleic Acids Res 27:1, 297-300), see Table 5, or the BIOBASE database “Transfac” (Biologische Datenbanken GmbH, Braunschweig). By such measures, variant transcription regulating nucleotide sequences as specified above can be artificially generated. Moreover, processes for mutagenizing nucleic acid sequences are known to the skilled worker and include, e.g., the use of oligonucleotides having one or more mutations compared with the region to be mutated (e.g. within the framework of a site-specific mutagenesis). Primers having approximately 15 to approximately 75 nucleotides or more are typically employed, with preferably about 10 to about 25 or more nucleotide residues being located on both sides of a sequence to be modified. Details and procedure for said mutagenesis processes are familiar to the skilled worker (Kunkel et al. (1987) Methods Enzymol 154:367-382; Tomic et al. (1990) Nucl Acids Res 12:1656; Upender et al. (1995) Biotechniques 18(1):29-30; U.S. Pat. No. 4,237,224). A mutagenesis can also be achieved by treatment of, for example, vectors comprising the transcription regulating nucleotide sequence of the invention with mutagenizing agents such as hydroxylamine. Mutagenesis also yields variant expression cassettes of the invention as specified above.

Generally, the transcription regulating nucleotide sequences and promoters of the invention may be employed to express a nucleic acid segment that is operably linked to said promoter such as, for example, an open reading frame, or a portion thereof, an anti-sense sequence, a sequence encoding for a double-stranded RNA sequence, or a transgene in plants.

Accordingly, a further embodiment of the present invention, the expression cassette of the present invention comprises at least one polynucleotide of interest being operatively linked to the transcription regulating nucleotide sequence and/or at least one a termination sequence or transcription. Thus, the expression cassette of the present invention, preferably, comprises a transcription regulating nucleotide sequence for the expression of at least one polynucleotide of interest. However, expression cassettes comprising transcription regulating nucleotide sequences with at least two, three, four or five or even more transcription regulating nucleotide sequences for polynucleotides of interest are also contemplated by the present invention.

The term “polynucleotide of interest” refers to a nucleic acid which shall be expressed under the control of the transcription regulating nucleotide sequence referred to herein. Preferably, a polynucleotide of interest encodes a polypeptide the presence of which is desired in a cell or plant seed as referred to herein. Such a polypeptide may be an enzyme which is required for the synthesis of seed storage compounds or may be a seed storage protein. It is to be understood that if the polynucleotide of interest encodes a polypeptide, transcription of the nucleic acid in RNA and translation of the transcribed RNA into the polypeptide may be required. A polynucleotide of interest, also preferably, includes biologically active RNA molecules and, more preferably, antisense RNAs, ribozymes, micro RNAs or siRNAs. For example, an undesired enzymatic activity in a seed can be reduced due to the seed specific expression of an antisense RNAs, ribozymes, micro RNAs or siRNAs. The underlying biological principles of action of the aforementioned biologically active RNA molecules are well known in the art. Moreover, the person skilled in the art is well aware of how to obtain nucleic acids which encode such biologically active RNA molecules. It is to be understood that the biologically active RNA molecules may be directly obtained by transcription of the nucleic acid of interest, i.e. without translation into a polypeptide. Preferably, at least one polynucleotide of interest to be expressed under the control of the transcription regulating nucleotide sequence of the present invention is heterologous in relation to said expression control sequence, i.e. it is not naturally under the control thereof, but said control has been produced in a non-natural manner (for example by genetic engineering processes)

An operable linkage may—for example—comprise an sequential arrangement of the transcription regulating nucleotide sequence of the invention (for example a sequence as described by SEQ ID NO: 1, 2, 3, 4, 5, 6, 7, 8, 9, 10, 11, 12, 13, 14, 15, 16, 17, or 18) with a nucleic acid sequence to be expressed, and—optionally—additional regulatory elements such as for example polyadenylation or transcription termination elements, enhancers, introns etc, in a way that the transcription regulating nucleotide sequence can fulfill its function in the process of expression the nucleic acid sequence of interest under the appropriate conditions. The term “appropriate conditions” mean preferably the presence of the expression cassette in a plant cell. Preferred are arrangements, in which the nucleic acid sequence of interest to be expressed is placed down-stream (i.e., in 3′-direction) of the transcription regulating nucleotide sequence of the invention in a way, that both sequences are covalently linked. Optionally additional sequences may be inserted in-between the two sequences. Such sequences may be for example linker or multiple cloning sites. Furthermore, sequences can be inserted coding for parts of fusion proteins (in case a fusion protein of the protein encoded by the nucleic acid of interest is intended to be expressed). Preferably, the distance between the polynucleotide of interest to be expressed and the transcription regulating nucleotide sequence of the invention is not more than 200 base pairs, preferably not more than 100 base pairs, more preferably no more than 50 base pairs.

An operable linkage in relation to any expression cassette or of the invention may be realized by various methods known in the art, comprising both in vitro and in vivo procedure. Thus, an expression cassette of the invention or an vector comprising such expression cassette may by realized using standard recombination and cloning techniques well known in the art (see e.g., Maniatis 1989; Silhavy 1984; Ausubel 1987).

An expression cassette may also be assembled by inserting a transcription regulating nucleotide sequence of the invention (for example a sequence as described by SEQ ID NO: 1, 2, 3, 4, 5, 6, 7, 8, 9, 10, 11, 12, 13, 14, 15, 16, 17, or 18) into the plant genome. Such insertion will result in an operable linkage to a nucleic acid sequence of interest, which as such already existed in the genome. By the insertion the nucleic acid of interest is expressed in a seed-preferential or seed-specific way due to the transcription regulating properties of the transcription regulating nucleotide sequence. The insertion may be directed or by chance. Preferably the insertion is directed and realized by for example homologous recombination. By this procedure a natural promoter may be exchanged against the transcription regulating nucleotide sequence of the invention, thereby modifying the expression profile of an endogenous gene. The transcription regulating nucleotide sequence may also be inserted in a way, that antisense mRNA of an endogenous gene is expressed, thereby inducing gene silencing.

Similar, a polynucleotide of interest to be expressed may by inserted into a plant genome comprising the transcription regulating nucleotide sequence in its natural genomic environment (i.e. linked to its natural gene) in a way that the inserted sequence becomes operably linked to the transcription regulating nucleotide sequence, thereby forming an expression cassette of the invention.

The expression cassette may be employed for numerous expression purposes such as for example expression of a protein, or expression of a antisense RNA, sense or double-stranded RNA. Preferably, expression of the nucleic acid sequence confers to the plant an agronomically valuable trait.

The polynucleotide of interest to be linked to the transcription regulating nucleotide sequence of the invention may be obtained from an insect resistance gene, a disease resistance gene such as, for example, a bacterial disease resistance gene, a fungal disease resistance gene, a viral disease resistance gene, a nematode disease resistance gene, a herbicide resistance gene, a gene affecting grain composition or quality, a nutrient utilization gene, a mycotoxin reduction gene, a male sterility gene, a selectable marker gene, a screenable marker gene, a negative selectable marker, a positive selectable marker, a gene affecting plant agronomic characteristics, i.e., yield, standability, and the like, or an environment or stress resistance gene, i.e., one or more genes that confer herbicide resistance or tolerance, insect resistance or tolerance, disease resistance or tolerance (viral, bacterial, fungal, oomycete, or nematode), stress tolerance or resistance (as exemplified by resistance or tolerance to drought, heat, chilling, freezing, excessive moisture, salt stress, or oxidative stress), increased yields, food content and makeup, physical appearance, male sterility, drydown, standability, prolificacy, starch properties or quantity, oil quantity and quality, amino acid or protein composition, and the like. By “resistant” is meant a plant, which exhibits substantially no phenotypic changes as a consequence of agent administration, infection with a pathogen, or exposure to stress. By “tolerant” is meant a plant, which, although it may exhibit some phenotypic changes as a consequence of infection, does not have a substantially decreased reproductive capacity or substantially altered metabolism.

Seed-specific transcription regulating nucleotide sequences (e.g., promoters) are useful for expressing a wide variety of genes including those which alter metabolic pathways, confer disease resistance, for protein production, e.g., antibody production, or to improve nutrient uptake and the like. Seed-specific transcription regulating nucleotide sequences (e.g., promoters) may be modified so as to be regulatable, e.g., inducible. The genes and transcription regulating nucleotide sequences (e.g., promoters) described hereinabove can be used to identify orthologous genes and their transcription regulating nucleotide sequences (e.g., promoters) which are also likely expressed in a particular tissue and/or development manner. Moreover, the orthologous transcription regulating nucleotide sequences (e.g., promoters) are useful to express linked open reading frames. In addition, by aligning the transcription regulating nucleotide sequences (e.g., promoters) of these orthologs, novel cis elements can be identified that are useful to generate synthetic transcription regulating nucleotide sequences (e.g., promoters).

Another object of the present invention refers to a vector comprising the expression cassette of the present invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., Cold Spring Harbor Laboratory, Cold Spring Harbor Laboratory Press, Cold Spring Harbor, N.Y., 1989) and other laboratory manuals, such as Methods in Molecular Biology, 1995, Vol. 44, Agrobacterium protocols, Ed.: Gartland and Davey, Humana Press, Totowa, N.J. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T-DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T-DNA sequences arranged on the same vector, binary systems are based on at least two vectors, one of which bears vir genes, but no T-DNA, while a second one bears T-DNA, but no vir gene. As a consequence, the last-mentioned vectors are relatively small, easy to manipulate and can be replicated both in E. coli and in Agrobacterium. An overview of binary vectors and their use can be found in Hellens et al, Trends in Plant Science (2000) 5, 446-451. Furthermore, by using appropriate cloning vectors, the expression cassette of the invention can be introduced into host cells or organisms such as plants or animals and, thus, be used in the transformation of plants, such as those which are published, and cited, in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225.

More preferably, the vector of the present invention is an expression vector. In such an expression vector, the expression cassette comprises a transcription regulating nucleotide sequence as specified above allowing for expression in eukaryotic cells or isolated fractions thereof. An expression vector may, in addition to the expression cassette of the invention, also comprise further regulatory elements including transcriptional as well as translational enhancers. Preferably, the expression vector is also a gene transfer or targeting vector. Expression vectors derived from viruses such as retroviruses, vaccinia virus, adeno-associated virus, herpes viruses, or bovine papilloma virus, may be used for delivery of the expression cassettes or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Suitable expression vector backbones are, preferably, derived from expression vectors known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), where glutathione S-transferase (GST), maltose E-binding protein and protein A, respectively, are fused with the nucleic acid of interest encoding a protein to be expressed. The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS174 (DE3) from a resident λ-prophage which harbors a T7 gn1 gene under the transcriptional control of the lacUV 5 promoter. Examples of vectors for expression in the yeast S. cerevisiae comprise pYepSec1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press:

Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23.

The vector of the present invention comprising the expression cassette will have to be propagated and amplified in a suitable organism, i.e. expression host.

Accordingly, another embodiment of the invention relates to transgenic host cells or non-human, transgenic organisms comprising an expression cassette of the invention. Preferred are prokaryotic and eukaryotic organisms. Both microorganism and higher organisms are comprised. Preferred microorganisms are bacteria, yeast, algae, and fungi. Preferred bacteria are those of the genus Escherichia, Erwinia, Agrobacterium, Flavobacterium, Alcaligenes, Pseudomonas, Bacillus or Cyanobacterim such as—for example—Synechocystis and other bacteria described in Brock Biology of Microorganisms Eighth Edition (pages A-8, A-9, A10 and A11). Most preferably the transgenic cells or non-human, transgenic organisms comprising an expression cassette of the invention is a plant cell or plant (as defined above), more preferably a plant used for oil production such as—for example—Brassica napus, Brassica juncea, Linum usitatissimum, soybean, Camelina or sunflower.

Especially preferred are microorganisms capable to infect plants and to transfer DNA into their genome, especially bacteria of the genus Agrobacterium, preferably Agrobacterium tumefaciens and rhizogenes. Preferred yeasts are Candida, Saccharomyces, Hansenula and Pichia. Preferred fungi are Aspergillus, Trichoderma, Ashbya, Neurospora, Fusarium, and Beauveria.

In a preferred embodiment of the present invention, the host cell relates to a plant cell, plant, a plant seed, a non-human animal or a multicellular micro-organism.

Accordingly, the present invention further refers to a transgenic plant cell, plant tissue, plant organ, or plant seed, comprising the expression cassette or the vector of the present invention.

The expression cassette or vector may be present in the cytoplasm of the organism or may be incorporated into the genome either heterologous or by homologous recombination. Host cells, in particular those obtained from plants or animals, may be introduced into a developing embryo in order to obtain mosaic or chimeric organisms, i.e. transgenic organisms, i.e. plants, comprising the host cells of the present invention. Suitable transgenic organisms are, preferably, all organisms which are suitable for the expression of recombinant genes.

The nature of the transgenic plant cells is not limited, for example, the plant cell can be a monocotyledonous plant cell, or a dicotyledonous plant cell. Preferably, the transgenic plant transgenic plant tissue, plant organ, plant or seed is a monocotyledonous plant or a plant cell, plant tissue, plant organ, plant seed from a monocotyledonous plant.

Examples of transgenic plant cells finding use with the invention include cells (or entire plants or plant parts) derived from the genera: Ananas, Musa, Vitis, Fragaria, Lotus, Medicago, Onobrychis, Trifolium, Trigonella, Vigna, Citrus, Carica, Persea, Prunus, Syragrus, Theobroma, Coffea, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Mangifera, Cichorium, Helianthus, Lactuca, Bromus, Asparagus, Antirrhinum, Heterocallis, Nemesia, Pelargonium, Panicum, Pennisetum, Ranunculus, Senecio, Salpiglossis, Cucurbita, Cucumis, Browaalia, Lolium, Malus, Apium, Gossypium, Vicia, Lathyrus, Lupinus, Pachyrhizus, Wisteria, Stizolobium, Agrostis, Phleum, Dactylis, Sorghum, Setaria, Zea, Oryza, Triticum, Secale, Avena, Hordeum, Saccharum, Poa, Festuca, Stenotaphrum, Cynodon, Coix, Olyreae, Phareae, Glycine, Pisum, Psidium, Passiflora, Cicer, Phaseolus, Lens, and Arachis

Preferably, the transgenic plant cells finding use with the invention include cells (or entire plants or plant parts) from the family of poaceae, such as the genera Hordeum, Secale, Avena, Sorghum, Andropogon, Holcus, Panicum, Oryza, Zea, Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum, Secale cereale, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cemuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum, Oryza sativa, Oryza latifolia, Zea mays, Triticum aestivum, Triticum durum, Triticum turgidum, Triticum hybemum, Triticum macha, Triticum sativum or Triticum vulgare.

In particular, preferred plants to be used as transgenic plants in accordance with the present invention are oil fruit crops which comprise large amounts of lipid compounds, such as peanut, oilseed rape, canola, sunflower, safflower, poppy, mustard, hemp, castor-oil plant, olive, sesame, Calendula, Punica, evening primrose, mullein, thistle, wild roses, hazelnut, almond, macadamia, avocado, bay, pumpkin/squash, linseed, soybean, pistachios, borage, trees (oil palm, coconut, walnut) or crops such as maize, wheat, rye, oats, triticale, rice, barley, cotton, cassava, pepper, Tagetes, Solanaceae plants such as potato, tobacco, eggplant and tomato, Vicia species, pea, alfalfa or bushy plants (coffee, cacao, tea), Salix species, and perennial grasses and fodder crops. Preferred plants according to the invention are oil crop plants such as peanut, oilseed rape, canola, sunflower, safflower, poppy, mustard, hemp, castor-oil plant, olive, Calendula, Punica, evening primrose, pumpkin/squash, linseed, soybean, borage, trees (oil palm, coconut).

In another aspect, the present invention relates to a method for producing a transgenic plant tissue, plant organ, plant or seed comprising
(a) introducing the expression cassette or the vector of the invention into a plant cell; and
(b) regenerating said plant cell to form a plant tissue, plant organ, plant or seed.

Expression cassettes can be introduced into plant cells in a number of art-recognized ways. Plant species may be transformed with the DNA construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. These techniques generally include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes of the invention. The general descriptions of plant expression vectors and reporter genes, and Agrobacterium and Agrobacterium-mediated gene transfer, can be found in Gruber et al. (1993).

Expression vectors containing genomic or synthetic fragments can be introduced into protoplasts or into intact tissues or isolated cells. Preferably expression vectors are introduced into intact tissue. General methods of culturing plant tissues are provided for example by Maki et al., (1993); and by Phillips et al. (1988). Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device. See, for example, Tomes et al. (1995). The vectors of the invention can not only be used for expression of structural genes but may also be used in exon-trap cloning, or promoter trap procedures to detect differential gene expression in varieties of tissues (Lindsey 1993; Auch & Reth 1990).

It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti 1985: Byrne 1987; Sukhapinda 1987; Lorz 1985; Potrykus, 1985; Park 1985: Hiei 1994). The use of T-DNA to transform plant cells has received extensive study and is amply described (EP 120516; Hoekema, 1985; Knauf, 1983; and An 1985). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (Fromm 1986) or high velocity ballistic bombardment with metal particles coated with the nucleic acid constructs (Kline 1987, and U.S. Pat. No. 4,945,050). Once transformed, the cells can be regenerated by those skilled in the art. Of particular relevance are the recently described methods to transform foreign genes into commercially important crops, such as rapeseed (De Block 1989), sunflower (Everett 1987), soybean (McCabe 1988; Hinchee 1988; Chee 1989; Christou 1989; EP 301749), rice (Hiei 1994), and corn (Gordon-Kamm 1990; Fromm 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway 1986), electroporation (Riggs 1986), Agrobacterium-mediated transformation (Hinchee 1988), direct gene transfer (Paszkowski 1984), and ballistic particle acceleration using devices available from Agracetus, Inc., Madison, Wis. And BioRad, Hercules, Calif. (see, for example, U.S. Pat. No. 4,945,050; and McCabe 1988). Also see, Weissinger 1988; Sanford 1987 (onion); Christou 1988 (soybean); McCabe 1988 (soybean); Datta 1990 (rice); Klein 1988 (maize); Klein 1988 (maize); Klein 1988 (maize); Fromm 1990 (maize); and Gordon-Kamm 1990 (maize); Svab 1990 (tobacco chloroplast); Koziel 1993 (maize); Shimamoto 1989 (rice); Christou 1991 (rice); European Patent Application EP 0 332 581 (orchardgrass and other Pooideae); Vasil 1993 (wheat); Weeks 1993 (wheat).

In another embodiment, a nucleotide sequence of the present invention is directly transformed into the plastid genome. Plastid transformation technology is extensively described in U.S. Pat. Nos. 5,451,513, 5,545,817, and 5,545,818, in PCT application no. WO 95/16783, and in McBride et al., 1994. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate orthologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab 1990; Staub 1992). This resulted in stable homoplasmic transformants at a frequency of approximately one per 100 bombardments of target leaves. The presence of cloning sites between these markers allowed creation of a plastid-targeting vector for introduction of foreign genes (Staub 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3N-adenyltransferase (Svab 1993). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

Agrobacterium tumefaciens cells containing a vector comprising an expression cassette of the present invention, wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known.

Various Agrobacterium strains can be employed, preferably disarmed Agrobacterium tumefaciens or rhizogenes strains. In a preferred embodiment, Agrobacterium strains for use in the practice of the invention include octopine strains, e.g., LBA4404 or agropine strains, e.g., EHA101 or EHA105. Suitable strains of A. tumefaciens for DNA transfer are for example EHA101[pEHA101] (Hood 1986), EHA105[pEHA105] (Li 1992), LBA4404[pAL4404] (Hoekema 1983), C58C1[pMP90] (Koncz & Schell 1986), and C58C1[pGV2260] (Deblaere 1985). Other suitable strains are Agrobacterium tumefaciens C58, a nopaline strain. Other suitable strains are A. tumefaciens C58C1 (Van Larebeke 1974), A136 (Watson 1975) or LBA4011 (Klapwijk 1980). In another preferred embodiment the soil-borne bacterium is a disarmed variant of Agrobacterium rhizogenes strain K599 (NCPPB 2659). Preferably, these strains are comprising disarmed plasmid variants of a Ti- or Ri-plasmid providing the functions required for T-DNA transfer into plant cells (e.g., the vir genes). In a preferred embodiment, the Agrobacterium strain used to transform the plant tissue pre-cultured with the plant phenolic compound contains a L,L-succinamopine type Ti-plasmid, preferably disarmed, such as pEHA101. In another preferred embodiment, the Agrobacterium strain used to transform the plant tissue pre-cultured with the plant phenolic compound contains an octopine-type Ti-plasmid, preferably disarmed, such as pAL4404. Generally, when using octopine-type Ti-plasmids or helper plasmids, it is preferred that the virF gene be deleted or inactivated (Jarschow 1991).

The method of the invention can also be used in combination with particular Agrobacterium strains, to further increase the transformation efficiency, such as Agrobacterium strains wherein the vir gene expression and/or induction thereof is altered due to the presence of mutant or chimeric virA or virG genes (e.g. Hansen 1994; Chen and Winans 1991; Scheeren-Groot, 1994). Preferred are further combinations of Agrobacterium tumefaciens strain LBA4404 (Hiei 1994) with super-virulent plasmids. These are preferably pTOK246-based vectors (Ishida 1996).

A binary vector or any other vector can be modified by common DNA recombination techniques, multiplied in E. coli, and introduced into Agrobacterium by e.g., electroporation or other transformation techniques (Mozo & Hooykaas 1991).

Agrobacterium is grown and used in a manner similar to that described in Ishida (1996). The vector comprising Agrobacterium strain may, for example, be grown for 3 days on YP medium (5 g/l yeast extract, 10 g/l peptone, 5 g/l NaCl, 15 g/l agar, pH 6.8) supplemented with the appropriate antibiotic (e.g., 50 mg/l spectinomycin). Bacteria are collected with a loop from the solid medium and resuspended. In a preferred embodiment of the invention, Agrobacterium cultures are started by use of aliquots frozen at −80° C.

The transformation of the target tissue (e.g., an immature embryo) by the Agrobacterium may be carried out by merely contacting the target tissue with the Agrobacterium. The concentration of Agrobacterium used for infection and co-cultivation may need to be varied. For example, a cell suspension of the Agrobacterium having a population density of approximately from 10⁵-10¹¹, preferably 10⁶ to 10¹⁰, more preferably about 10⁸ cells or cfu/ml is prepared and the target tissue is immersed in this suspension for about 3 to 10 minutes. The resulting target tissue is then cultured on a solid medium for several days together with the Agrobacterium.

Preferably, the bacterium is employed in concentration of 10⁶ to 10¹⁰ cfu/ml. In a preferred embodiment for the co-cultivation step about 1 to 10 μl of a suspension of the soil-borne bacterium (e.g., Agrobacteria) in the co-cultivation medium are directly applied to each target tissue explant and air-dried. This is saving labor and time and is reducing unintended Agrobacterium-mediated damage by excess Agrobacterium usage.

For Agrobacterium treatment, the bacteria are resuspended in a plant compatible co-cultivation medium. Supplementation of the co-culture medium with antioxidants (e.g., silver nitrate), phenol-absorbing compounds (like polyvinylpyrrolidone, Perl 1996) or thiol compounds (e.g., dithiothreitol, L-cysteine, Olhoft 2001) which can decrease tissue necrosis due to plant defence responses (like phenolic oxidation) may further improve the efficiency of Agrobacterium-mediated transformation. In another preferred embodiment, the co-cultivation medium of comprises least one thiol compound, preferably selected from the group consisting of sodium thiolsulfate, dithiotrietol (DTT) and cysteine. Preferably the concentration is between about 1 mM and 10 mM of L-Cysteine, 0.1 mM to 5 mM DTT, and/or 0.1 mM to 5 mM sodium thiolsulfate. Preferably, the medium employed during co-cultivation comprises from about 1 μM to about 10 μM of silver nitrate and from about 50 mg/L to about 1,000 mg/L of L-Cystein. This results in a highly reduced vulnerability of the target tissue against Agrobacterium-mediated damage (such as induced necrosis) and highly improves overall transformation efficiency.

Various vector systems can be used in combination with Agrobacteria. Preferred are binary vector systems. Common binary vectors are based on “broad host range”-plasmids like pRK252 (Bevan 1984) or pTJS75 (Watson 1985) derived from the P-type plasmid RK2. Most of these vectors are derivatives of pBIN19 (Bevan 1984). Various binary vectors are known, some of which are commercially available such as, for example, pBI101.2 or pBIN19 (Clontech Laboratories, Inc. USA). Additional vectors were improved with regard to size and handling (e.g. pPZP; Hajdukiewicz 1994). Improved vector systems are described also in WO 02/00900.

Methods using either a form of direct gene transfer or Agrobacterium-mediated transfer usually, but not necessarily, are undertaken with a selectable marker, which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptII gene which confers resistance to kanamycin and related antibiotics (Messing & Vierra, 1982; Bevan 1983), the bar gene which confers resistance to the herbicide phosphinothricin (White 1990, Spencer 1990), the hph gene which confers resistance to the antibiotic hygromycin (Blochlinger & Diggelmann), and the dhfr gene, which confers resistance to methotrexate (Bourouis 1983).

Methods for the production and further characterization of stably transformed plants are well-known to the person skilled in the art. As an example, transgenic plant cells are placed in an appropriate selective medium for selection of transgenic cells, which are then grown to callus. Shoots are grown from callus. Plantlets are generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA, which has been introduced. Components of DNA constructs including transcription cassettes of this invention may be prepared from sequences, which are native (endogenous) or foreign (exogenous) to the host. By “foreign” it is meant that the sequence is not found in the wild-type host into which the construct is introduced. Heterologous constructs will contain at least one region, which is not native to the gene from which the transcription-initiation-region is derived.

To confirm the presence of the transgenes in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR or TaqMan; “biochemical” assays, such as detecting the presence of a protein product, e.g., by immunological means (ELISAs and Western blots) or by enzymatic function; plant part assays, such as seed assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The presence of nucleic acid elements introduced through the methods of this invention may be determined by polymerase chain reaction (PCR). Using these technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the, genome, i.e., whether transformants are of independent origin. It is contemplated that using PCR techniques it would be possible to clone fragments of the host genomic DNA adjacent to an introduced preselected DNA segment. Known methods of PCR include, but are not limited to, methods using paired primers, nested primers, single specific primers, degenerate primers, gene-specific primers, vector-specific primers, partially mismatched primers, and the like.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected, DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer 1992); Laursen 1994) indicating stable inheritance of the gene. The non-chimeric nature of the callus and the parental transformants (R₀) was suggested by germline transmission and the identical Southern blot hybridization patterns and intensities of the transforming DNA in callus, R₀ plants and R₁ progeny that segregated for the transformed gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

The following section provides examples of particular polynucleotides of interest, which can be operably linked to the expression cassette of the present invention.

### 1. Exemplary Transgenes

### 1.1. Herbicide Resistance

The genes encoding phosphinothricin acetyltransferase (bar and pat), glyphosate tolerant EPSP synthase genes, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are good examples of herbicide resistant genes for use in transformation. The bar and pat genes code for an enzyme, phosphinothricin acetyltransferase (PAT), which inactivates the herbicide phosphinothricin and prevents this compound from inhibiting glutamine synthetase enzymes. The enzyme 5-enolpyruvylshikimate 3-phosphate synthase (EPSP Synthase), is normally inhibited by the herbicide N-(phosphonomethyl) glycine (glyphosate). However, genes are known that encode glyphosate-resistant EPSP Synthase enzymes. The deh gene encodes the enzyme dalapon dehalogenase and confers resistance to the herbicide dalapon. The bxn gene codes for a specific nitrilase enzyme that converts bromoxynil to a non-herbicidal degradation product.
1.2 Insect Resistance

An important aspect of the present invention concerns the introduction of insect resistance-conferring genes into plants. Potential insect resistance genes, which can be introduced, include Bacillus thuringiensis crystal toxin genes or Bt genes (Watrud 1985). Bt genes may provide resistance to lepidopteran or coleopteran pests such as European Corn Borer (ECB) and corn rootworm (CRW). Preferred Bt toxin genes for use in such embodiments include the CryIA(b) and CryIA(c) genes. Endotoxin genes from other species of B. thuringiensis, which affect insect growth or development, may also be employed in this regard. Protease inhibitors may also provide insect resistance (Johnson 1989), and will thus have utility in plant transformation. The use of a protease inhibitor II gene, pinII, from tomato or potato is envisioned to be particularly useful. Even more advantageous is the use of a pinII gene in combination with a Bt toxin gene, the combined effect of which has been discovered by the present inventors to produce synergistic insecticidal activity. Other genes, which encode inhibitors of the insects' digestive system, or those that encode enzymes or co-factors that facilitate the production of inhibitors, may also be useful. Cystatin and amylase inhibitors, such as those from wheat and barley, may exemplify this group.

Also, genes encoding lectins may confer additional or alternative insecticide properties. Lectins (originally termed phytohemagglutinins) are multivalent carbohydrate-binding proteins, which have the ability to agglutinate red blood cells from a range of species. Lectins have been identified recently as insecticidal agents with activity against weevils, ECB and rootworm (Murdock 1990; Czapla & Lang, 1990). Lectin genes contemplated to be useful include, for example, barley and wheat germ agglutinin (WGA) and rice lectins (Gatehouse 1984), with WGA being preferred.

Genes controlling the production of large or small polypeptides active against insects when introduced into the insect pests, such as, e.g., lytic peptides, peptide hormones and toxins and venoms, form another aspect of the invention. For example, it is contemplated, that the expression of juvenile hormone esterase, directed towards specific insect pests, may also result in insecticidal activity, or perhaps cause cessation of metamorphosis (Hammock 1990).

Transgenic plants expressing genes, which encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant maize plants. Genes that code for activities that affect insect molting, such those affecting the production of ecdysteroid UDP-glucosyl transferase, also fall within the scope of the useful transgenes of the present invention.

Genes that code for enzymes that facilitate the production of compounds that reduce the nutritional quality of the host plant to insect pests are also encompassed by the present invention. It may be possible, for instance, to confer insecticidal activity on a plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, further embodiments of the invention concern transgenic plants with enhanced lipoxygenase activity which may be resistant to insect feeding.

The present invention also provides methods and compositions by which to achieve qualitative or quantitative changes in plant secondary metabolites. One example concerns transforming plants to produce DIMBOA which, it is contemplated, will confer resistance to European corn borer, rootworm and several other maize insect pests. Candidate genes that are particularly considered for use in this regard include those genes at the bx locus known to be involved in the synthetic DIMBOA pathway (Dunn 1981). The introduction of genes that can regulate the production of maysin, and genes involved in the production of dhurrin in sorghum, is also contemplated to be of use in facilitating resistance to earworm and rootworm, respectively.

Tripsacum dactyloides is a species of grass that is resistant to certain insects, including corn rootworm. It is anticipated that genes encoding proteins that are toxic to insects or are involved in the biosynthesis of compounds toxic to insects will be isolated from Tripsacum and that these novel genes will be useful in conferring resistance to insects. It is known that the basis of insect resistance in Tripsacum is genetic, because said resistance has been transferred to Zea mays via sexual crosses (Branson & Guss, 1972).

Further genes encoding proteins characterized as having potential insecticidal activity may also be used as transgenes in accordance herewith. Such genes include, for example, the cowpea trypsin inhibitor (CpTI; Hilder 1987), which may be used as a rootworm deterrent; genes encoding avermectin (Campbell 1989; Ikeda 1987) which may prove particularly useful as a corn rootworm deterrent; ribosome inactivating protein genes; and even genes that regulate plant structures. Transgenic maize including anti-insect antibody genes and genes that code for enzymes that can covert a non-toxic insecticide (pro-insecticide) applied to the outside of the plant into an insecticide inside the plant are also contemplated.

### 1.3 Environment or Stress Resistance

Improvement of a plant's ability to tolerate various environmental stresses such as, but not limited to, drought, excess moisture, chilling, freezing, high temperature, salt, and oxidative stress, can also be effected through expression of heterologous, or overexpression of homologous genes. Benefits may be realized in terms of increased resistance to freezing temperatures through the introduction of an “antifreeze” protein such as that of the Winter Flounder (Cutler 1989) or synthetic gene derivatives thereof. Improved chilling tolerance may also be conferred through increased expression of glycerol-3-phosphate acetyltransferase in chloroplasts (Murata 1992; Wolter 1992). Resistance to oxidative stress (often exacerbated by conditions such as chilling temperatures in combination with high light intensities) can be conferred by expression of superoxide dismutase (Gupta 1993), and may be improved by glutathione reductase (Bowler 1992). Such strategies may allow for tolerance to freezing in newly emerged fields as well as extending later maturity higher yielding varieties to earlier relative maturity zones.

Expression of novel genes that favorably effect plant water content, total water potential, osmotic potential, and turgor can enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments, and perform in a relatively superior manner. In this aspect of the invention it is proposed, for example, that the expression of a gene encoding the biosynthesis of osmotically active solutes can impart protection against drought. Within this class of genes are DNAs encoding mannitol dehydrogenase (Lee and Saier, 1982) and trehalose-6-phosphate synthase (Kaasen 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski 1992).

Similarly, the efficacy of other metabolites in protecting either enzyme function (e.g. alanopine or propionic acid) or membrane integrity (e.g., alanopine) has been documented (Loomis 1989), and therefore expression of gene encoding the biosynthesis of these compounds can confer drought resistance in a manner similar to or complimentary to mannitol. Other examples of naturally occurring metabolites that are osmotically active and/or provide some direct protective effect during drought and/or desiccation include sugars and sugar derivatives such as fructose, erythritol (Coxson 1992), sorbitol, dulcitol (Karsten 1992), glucosylglycerol (Reed 1984; Erdmann 1992), sucrose, stachyose (Koster & Leopold 1988; Blackman 1992), ononitol and pinitol (Vernon & Bohnert 1992), and raffinose (Bernal-Lugo & Leopold 1992). Other osmotically active solutes, which are not sugars, include, but are not limited to, proline and glycine-betaine (Wyn-Jones and Storey, 1981). Continued canopy growth and increased reproductive fitness during times of stress can be augmented by introduction and expression of genes such as those controlling the osmotically active compounds discussed above and other such compounds, as represented in one exemplary embodiment by the enzyme myoinositol 0-methyltransferase.

It is contemplated that the expression of specific proteins may also increase drought tolerance. Three classes of Late Embryogenic Proteins have been assigned based on structural similarities (see Dure 1989). All three classes of these proteins have been demonstrated in maturing (i.e., desiccating) seeds. Within these 3 types of proteins, the Type-II (dehydrin-type) have generally been implicated in drought and/or desiccation tolerance in vegetative plant parts (e.g. Mundy and Chua, 1988; Piatkowski 1990; Yamaguchi-Shinozaki 1992). Recently, expression of a Type-III LEA (HVA-1) in tobacco was found to influence plant height, maturity and drought tolerance (Fitzpatrick, 1993). Expression of structural genes from all three groups may therefore confer drought tolerance. Other types of proteins induced during water stress include thiol proteases, aldolases and transmembrane transporters (Guerrero 1990), which may confer various protective and/or repair-type functions during drought stress. The expression of a gene that effects lipid biosynthesis and hence membrane composition can also be useful in conferring drought resistance on the plant.

Many genes that improve drought resistance have complementary modes of action. Thus, combinations of these genes might have additive and/or synergistic effects in improving drought resistance in maize. Many of these genes also improve freezing tolerance (or resistance); the physical stresses incurred during freezing and drought are similar in nature and may be mitigated in similar fashion. Benefit may be conferred via constitutive expression or tissue-specific of these genes, but the preferred means of expressing these novel genes may be through the use of a turgor-induced promoter (such as the promoters for the turgor-induced genes described in Guerrero et al. 1990 and Shagan 1993). Spatial and temporal expression patterns of these genes may enable maize to better withstand stress.

Expression of genes that are involved with specific morphological traits that allow for increased water extractions from drying soil would be of benefit. For example, introduction and expression of genes that alter root characteristics may enhance water uptake. Expression of genes that enhance reproductive fitness during times of stress would be of significant value. For example, expression of DNAs that improve the synchrony of pollen shed and receptiveness of the female flower parts, i.e., silks, would be of benefit. In addition, expression of genes that minimize kernel abortion during times of stress would increase the amount of grain to be harvested and hence be of value. Regulation of cytokinin levels in monocots, such as maize, by introduction and expression of an isopentenyl transferase gene with appropriate regulatory sequences can improve monocot stress resistance and yield (Gan 1995).

Given the overall role of water in determining yield, it is contemplated that enabling plants to utilize water more efficiently, through the introduction and expression of novel genes, will improve overall performance even when soil water availability is not limiting. By introducing genes that improve the ability of plants to maximize water usage across a full range of stresses relating to water availability, yield stability or consistency of yield performance may be realized.

Improved protection of the plant to abiotic stress factors such as drought, heat or chill, can also be achieved—for example—by overexpressing antifreeze polypeptides from Myoxocephalus Scorpius (WO 00/00512), Myoxocephalus octodecemspinosus, the Arabidopsis thaliana transcription activator CBF1, glutamate dehydrogenases (WO 97/12983, WO 98/11240), calcium-dependent protein kinase genes (WO 98/26045), calcineurins (WO 99/05902), casein kinase from yeast (WO 02/052012), farnesyltransferases (WO 99/06580; Pei Z M et al. (1998) Science 282:287-290), ferritin (Deak M et al. (1999) Nature Biotechnology 17:192-196), oxalate oxidase (WO 99/04013; Dunwell J M (1998) Biotechn Genet Eng Rev 15:1-32), DREB1A factor (“dehydration response element B 1A”; Kasuga M et al. (1999) Nature Biotech 17:276-286), genes of mannitol or trehalose synthesis such as trehalose-phosphate synthase or trehalose-phosphate phosphatase (WO 97/42326) or by inhibiting genes such as trehalase (WO 97/50561).

### 1.4 Disease Resistance

It is proposed that increased resistance to diseases may be realized through introduction of genes into plants period. It is possible to produce resistance to diseases caused, by viruses, bacteria, fungi, root pathogens, insects and nematodes. It is also contemplated that control of mycotoxin producing organisms may be realized through expression of introduced genes.

Resistance to viruses may be produced through expression of novel genes. For example, it has been demonstrated that expression of a viral coat protein in a transgenic plant can impart resistance to infection of the plant by that virus and perhaps other closely related viruses (Cuozzo 1988, Hemenway 1988, Abel 1986). It is contemplated that expression of antisense genes targeted at essential viral functions may impart resistance to said virus. For example, an antisense gene targeted at the gene responsible for replication of viral nucleic acid may inhibit said replication and lead to resistance to the virus. It is believed that interference with other viral functions through the use of antisense genes may also increase resistance to viruses. Further it is proposed that it may be possible to achieve resistance to viruses through other approaches, including, but not limited to the use of satellite viruses.

It is proposed that increased resistance to diseases caused by bacteria and fungi may be realized through introduction of novel genes. It is contemplated that genes encoding so-called “peptide antibiotics,” pathogenesis related (PR) proteins, toxin resistance, and proteins affecting host-pathogen interactions such as morphological characteristics will be useful. Peptide antibiotics are polypeptide sequences, which are inhibitory to growth of bacteria and other microorganisms. For example, the classes of peptides referred to as cecropins and magainins inhibit growth of many species of bacteria and fungi. It is proposed that expression of PR proteins in plants may be useful in conferring resistance to bacterial disease. These genes are induced following pathogen attack on a host plant and have been divided into at least five classes of proteins (Bol 1990). Included amongst the PR proteins are beta-1,3-glucanases, chitinases, and osmotin and other proteins that are believed to function in plant resistance to disease organisms. Other genes have been identified that have antifungal properties, e.g., UDA (stinging nettle lectin) and hevein (Broakgert 1989; Barkai-Golan 1978). It is known that certain plant diseases are caused by the production of phytotoxins. Resistance to these diseases could be achieved through expression of a novel gene that encodes an enzyme capable of degrading or otherwise inactivating the phytotoxin. Expression novel genes that alter the interactions between the host plant and pathogen may be useful in reducing the ability the disease organism to invade the tissues of the host plant, e.g., an increase in the waxiness of the leaf cuticle or other morphological characteristics.

Plant parasitic nematodes are a cause of disease in many plants. It is proposed that it would be possible to make the plant resistant to these organisms through the expression of novel genes. It is anticipated that control of nematode infestations would be accomplished by altering the ability of the nematode to recognize or attach to a host plant and/or enabling the plant to produce nematicidal compounds, including but not limited to proteins.

Furthermore, a resistance to fungi, insects, nematodes and diseases, can be achieved by targeted accumulation of certain metabolites or proteins. Such proteins include but are not limited to glucosinolates (defense against herbivores), chitinases or glucanases and other enzymes which destroy the cell wall of parasites, ribosome-inactivating proteins (RIPs) and other proteins of the plant resistance and stress reaction as are induced when plants are wounded or attacked by microbes, or chemically, by, for example, salicylic acid, jasmonic acid or ethylene, or lysozymes from nonplant sources such as, for example, T4-lysozyme or lysozyme from a variety of mammals, insecticidal proteins such as Bacillus thuringiensis endotoxin, a-amylase inhibitor or protease inhibitors (cowpea trypsin inhibitor), lectins such as wheatgerm agglutinin, RNAses or ribozymes. Further examples are nucleic acids which encode the Trichoderma harzianum chit42 endochitinase (GenBank Acc. No.: S78423) or the N-hydroxylating, multi-functional cytochrome P-450 (CYP79) protein from Sorghum bicolor (GenBank Acc. No.: U32624), or functional equivalents of these. The accumulation of glucosinolates as protection from pests (Rask L et al. (2000) Plant Mol Biol 42:93-113; Menard R et al. (1999) Phytochemistry 52:29-35), the expression of Bacillus thuringiensis endotoxins (Vaeck et al. (1987) Nature 328:33-37) or the protection against attack by fungi, by expression of chitinases, for example from beans (Broglie et al. (1991) Science 254:1194-1197), is advantageous. Resistance to pests such as, for example, the rice pest Nilaparvata lugens in rice plants can be achieved by expressing the snowdrop (Galanthus nivalis) lectin agglutinin (Rao et al. (1998) Plant J 15(4):469-77). The expression of synthetic cryIA(b) and cryIA(c) genes, which encode lepidoptera-specific Bacillus thuringiensis D-endotoxins can bring about a resistance to insect pests in various plants (Goyal R K et al. (2000) Crop Protection 19(5):307-312). Further target genes which are suitable for pathogen defense comprise “polygalacturonase-inhibiting protein” (PGIP), thaumatine, invertase and antimicrobial peptides such as lactoferrin (Lee T J et al. (2002) J Amer Soc Horticult Sci 127(2):158-164). Other nucleic acid sequences which may be advantageously used herein include traits for insect control (U.S. Pat. Nos. 6,063,597; 6,063,756; 6,093,695; 5,942,664; and 6,110,464), fungal disease resistance (U.S. Pat. Nos. 5,516,671; 5,773,696; 6,121,436; 6,316,407; and 6,506,962), virus resistance (U.S. Pat. Nos. 5,304,730 and 6,013,864), nematode resistance (U.S. Pat. No. 6,228,992), and bacterial disease resistance (U.S. Pat. No. 5,516,671).

### 1.5 Mycotoxin Reduction/Elimination

Production of mycotoxins, including aflatoxin and fumonisin, by fungi associated with plants is a significant factor in rendering the grain not useful. These fungal organisms do not cause disease symptoms and/or interfere with the growth of the plant, but they produce chemicals (mycotoxins) that are toxic to animals. Inhibition of the growth of these fungi would reduce the synthesis of these toxic substances and, therefore, reduce grain losses due to mycotoxin contamination. Novel genes may be introduced into plants that would inhibit synthesis of the mycotoxin without interfering with fungal growth. Expression of a novel gene, which encodes an enzyme capable of rendering the mycotoxin nontoxic, would be useful in order to achieve reduced mycotoxin contamination of grain. The result of any of the above mechanisms would be a reduced presence of mycotoxins on grain.

### 1.6 Grain Composition or Quality

Genes may be introduced into plants, particularly commercially important cereals such as maize, wheat or rice, to improve the grain for which the cereal is primarily grown. A wide range of novel transgenic plants produced in this manner may be envisioned depending on the particular end use of the grain.

For example, the largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes but in no way provide an exhaustive list of possibilities.

The protein of many cereal grains is suboptimal for feed and food purposes especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after the grain is supplemented with other inputs for feed formulations. For example, when the grain is supplemented with soybean meal to meet lysine requirements, methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

One mechanism for increasing the biosynthesis of the amino acids is to introduce genes that deregulate the amino acid biosynthetic pathways such that the plant can no longer adequately control the levels that are produced. This may be done by deregulating or bypassing steps in the amino acid biosynthetic pathway that are normally regulated by levels of the amino acid end product of the pathway. Examples include the introduction of genes that encode deregulated versions of the enzymes aspartokinase or dihydrodipicolinic acid (DHDP)-synthase for increasing lysine and threonine production, and anthranilate synthase for increasing tryptophan production. Reduction of the catabolism of the amino acids may be accomplished by introduction of DNA sequences that reduce or eliminate the expression of genes encoding enzymes that catalyse steps in the catabolic pathways such as the enzyme lysine-ketoglutarate reductase.

The protein composition of the grain may be altered to improve the balance of amino acids in a variety of ways including elevating expression of native proteins, decreasing expression of those with poor composition, changing the composition of native proteins, or introducing genes encoding entirely new proteins possessing superior composition. DNA may be introduced that decreases the expression of members of the zein family of storage proteins. This DNA may encode ribozymes or antisense sequences directed to impairing expression of zein proteins or expression of regulators of zein expression such as the opaque-2 gene product. The protein composition of the grain may be modified through the phenomenon of cosuppression, i.e., inhibition of expression of an endogenous gene through the expression of an identical structural gene or gene fragment introduced through transformation (Goring 1991). Additionally, the introduced DNA may encode enzymes, which degrade zeines. The decreases in zein expression that are achieved may be accompanied by increases in proteins with more desirable amino acid composition or increases in other major seed constituents such as starch. Alternatively, a chimeric gene may be introduced that comprises a coding sequence for a native protein of adequate amino acid composition such as for one of the globulin proteins or 10 kD zein of maize and a promoter or other regulatory sequence designed to elevate expression of said protein. The coding sequence of said gene may include additional or replacement codons for essential amino acids. Further, a coding sequence obtained from another species, or, a partially or completely synthetic sequence encoding a completely unique peptide sequence designed to enhance the amino acid composition of the seed may be employed.

The introduction of genes that alter the oil content of the grain may be of value. Increases in oil content may result in increases in metabolizable energy content and density of the seeds for uses in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, beta-ketoacyl-ACP synthase, plus other well-known fatty acid biosynthetic activities. Other possibilities are genes that encode proteins that do not possess enzymatic activity such as acyl carrier protein. Additional examples include 2-acetyltransferase, oleosin pyruvate dehydrogenase complex, acetyl CoA synthetase, ATP citrate lyase, ADP-glucose pyrophosphorylase and genes of the carnitine-CoA-acetyl-CoA shuttles. It is anticipated that expression of genes related to oil biosynthesis will be targeted to the plastid, using a plastid transit peptide sequence and preferably expressed in the seed embryo. Genes may be introduced that alter the balance of fatty acids present in the oil providing a more healthful or nutritive feedstuff. The introduced DNA may also encode sequences that block expression of enzymes involved in fatty acid biosynthesis, altering the proportions of fatty acids present in the grain such as described below. Genes may be introduced that enhance the nutritive value of the starch component of the grain, for example by increasing the degree of branching, resulting in improved utilization of the starch in cows by delaying its metabolism.

Besides affecting the major constituents of the grain, genes may be introduced that affect a variety of other nutritive, processing, or other quality aspects of the grain as used for feed or food. For example, pigmentation of the grain may be increased or decreased. Enhancement and stability of yellow pigmentation is desirable in some animal feeds and may be achieved by introduction of genes that result in enhanced production of xanthophylls and carotenes by eliminating rate-limiting steps in their production. Such genes may encode altered forms of the enzymes phytoene synthase, phytoene desaturase, or lycopene synthase. Alternatively, unpigmented white corn is desirable for production of many food products and may be produced by the introduction of DNA, which blocks or eliminates steps in pigment production pathways.

Feed or food comprising some cereal grains possesses insufficient quantities of vitamins and must be supplemented to provide adequate nutritive value. Introduction of genes that enhance vitamin biosynthesis in seeds may be envisioned including, for example, vitamins A, E, B12, choline, and the like. For example, maize grain also does not possess sufficient mineral content for optimal nutritive value. Genes that affect the accumulation or availability of compounds containing phosphorus, sulfur, calcium, manganese, zinc, and iron among others would be valuable. An example may be the introduction of a gene that reduced phytic acid production or encoded the enzyme phytase, which enhances phytic acid breakdown. These genes would increase levels of available phosphate in the diet, reducing the need for supplementation with mineral phosphate.

Numerous other examples of improvement of cereals for feed and food purposes might be described. The improvements may not even necessarily involve the grain, but may, for example, improve the value of the grain for silage. Introduction of DNA to accomplish this might include sequences that alter lignin production such as those that result in the “brown midrib” phenotype associated with superior feed value for cattle.

In addition to direct improvements in feed or food value, genes may also be introduced which improve the processing of grain and improve the value of the products resulting from the processing. The primary method of processing certain grains such as maize is via wetmilling. Maize may be improved though the expression of novel genes that increase the efficiency and reduce the cost of processing such as by decreasing steeping time.

Improving the value of wetmilling products may include altering the quantity or quality of starch, oil, corn gluten meal, or the components of corn gluten feed. Elevation of starch may be achieved through the identification and elimination of rate limiting steps in starch biosynthesis or by decreasing levels of the other components of the grain resulting in proportional increases in starch. An example of the former may be the introduction of genes encoding ADP-glucose pyrophosphorylase enzymes with altered regulatory activity or which are expressed at higher level. Examples of the latter may include selective inhibitors of, for example, protein or oil biosynthesis expressed during later stages of kernel development.

The properties of starch may be beneficially altered by changing the ratio of amylose to amylopectin, the size of the starch molecules, or their branching pattern. Through these changes a broad range of properties may be modified which include, but are not limited to, changes in gelatinization temperature, heat of gelatinization, clarity of films and pastes, Theological properties, and the like. To accomplish these changes in properties, genes that encode granule-bound or soluble starch synthase activity or branching enzyme activity may be introduced alone or combination. DNA such as antisense constructs may also be used to decrease levels of endogenous activity of these enzymes. The introduced genes or constructs may possess regulatory sequences that time their expression to specific intervals in starch biosynthesis and starch granule development. Furthermore, it may be advisable to introduce and express genes that result in the in vivo derivatization, or other modification, of the glucose moieties of the starch molecule. The covalent attachment of any molecule may be envisioned, limited only by the existence of enzymes that catalyze the derivatizations and the accessibility of appropriate substrates in the starch granule. Examples of important derivations may include the addition of functional groups such as amines, carboxyls, or phosphate groups, which provide sites for subsequent in vitro derivatizations or affect starch properties through the introduction of ionic charges. Examples of other modifications may include direct changes of the glucose units such as loss of hydroxyl groups or their oxidation to aldehyde or carboxyl groups.

Oil is another product of wetmilling of corn and other grains, the value of which may be improved by introduction and expression of genes. The quantity of oil that can be extracted by wetmilling may be elevated by approaches as described for feed and food above. Oil properties may also be altered to improve its performance in the production and use of cooking oil, shortenings, lubricants or other oil-derived products or improvement of its health attributes when used in the food-related applications. Novel fatty acids may also be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids and the lipids possessing them or by increasing levels of native fatty acids while possibly reducing levels of precursors. Alternatively DNA sequences may be introduced which slow or block steps in fatty acid biosynthesis resulting in the increase in precursor fatty acid intermediates. Genes that might be added include desaturases, epoxidases, hydratases, dehydratases, and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids.

Improvements in the other major cereal wetmilling products, gluten meal and gluten feed, may also be achieved by the introduction of genes to obtain novel plants. Representative possibilities include but are not limited to those described above for improvement of food and feed value.

In addition it may further be considered that the plant be used for the production or manufacturing of useful biological compounds that were either not produced at all, or not produced at the same level, in the plant previously. The novel plants producing these compounds are made possible by the introduction and expression of genes by transformation methods. The possibilities include, but are not limited to, any biological compound which is presently produced by any organism such as proteins, nucleic acids, primary and intermediary metabolites, carbohydrate polymers, etc. The compounds may be produced by the plant, extracted upon harvest and/or processing, and used for any presently recognized useful purpose such as pharmaceuticals, fragrances, industrial enzymes to name a few.

Further possibilities to exemplify the range of grain traits or properties potentially encoded by introduced genes in transgenic plants include grain with less breakage susceptibility for export purposes or larger grit size when processed by dry milling through introduction of genes that enhance gamma-zein synthesis, popcorn with improved popping, quality and expansion volume through genes that increase pericarp thickness, corn with whiter grain for food uses though introduction of genes that effectively block expression of enzymes involved in pigment production pathways, and improved quality of alcoholic beverages or sweet corn through introduction of genes which affect flavor such as the shrunken gene (encoding sucrose synthase) for sweet corn.

### 1.7 Tuber or Seed Composition or Quality

Various traits can be advantageously expressed especially in seeds or tubers to improve composition or quality. Useful nucleic acid sequences that can be combined with the promoter nucleic acid sequence of the present invention and provide improved end-product traits include, without limitation, those encoding seed storage proteins, fatty acid pathway enzymes, tocopherol biosynthetic enzymes, amino acid biosynthetic enzymes, and starch branching enzymes. A discussion of exemplary heterologous DNAs useful for the modification of plant phenotypes may be found in, for example, U.S. Pat. Nos. 6,194,636; 6,207,879; 6,232,526; 6,426,446; 6,429,357; 6,433,252; 6,437,217; 6,515,201; and 6,583,338 and PCT Publication WO 02/057471, each of which is specifically incorporated herein by reference in its entirety. Such traits include but are not limited to:
Expression of metabolic enzymes for use in the food-and-feed sector, for example of phytases and cellulases. Especially preferred are nucleic acids such as the artificial cDNA, which encodes a microbial phytase (GenBank Acc. No.: A19451) or functional equivalents thereof.
Expression of genes, which bring about an accumulation of fine chemicals such as of tocopherols, tocotrienols or carotenoids. An example, which may be mentioned is phytoene desaturase. Preferred are nucleic acids, which encode the Narcissus pseudonarcissus photoene desaturase (GenBank Acc. No.: X78815) or functional equivalents thereof. Preferred tocopherol biosynthetic enzymes include tyrA, slr1736, ATPT2, dxs, dxr, GGPPS, HPPD, GMT, MT1, tMT2, AANT1, slr 1737, and an antisense construct for homogentisic acid dioxygenase (Kridl et al., Seed Sci. Res., 1:209:219 (1991); Keegstra, Cell, 56(2):247-53 (1989); Nawrath et al., Proc. Natl. Acad. Sci. USA, 91:12760-12764 (1994); Xia et al., J. Gen. Microbiol., 138:1309-1316 (1992); Lois et al., Proc. Natl. Acad. Sci. USA, 95 (5):2105-2110 (1998); Takahashi et al., Proc. Natl. Acad. Sci. USA, 95(17):9879-9884 (1998); Norris et al., Plant Physiol., 117:1317-1323 (1998); Bartley and Scolnik, Plant Physiol., 104:1469-1470 (1994); Smith et al., Plant J., 11:83-92 (1997); WO 00/32757; WO 00/10380; Saint Guily et al., Plant Physiol., 100(2):1069-1071 (1992); Sato et al., J. DNA Res., 7(1):31-63 (2000)) all of which are incorporated herein by reference.
starch production (U.S. Pat. Nos. 5,750,876 and 6,476,295), high protein production (U.S. Pat. No. 6,380,466), fruit ripening (U.S. Pat. No. 5,512,466), enhanced animal and human nutrition (U.S. Pat. Nos. 5,985,605 and 6,171,640), biopolymers (U.S. Pat. No. 5,958,745 and U.S. Patent Publication No. 2003/0028917), environmental stress resistance (U.S. Pat. No. 6,072,103), pharmaceutical peptides (U.S. Pat. No. 6,080,560), improved processing traits (U.S. Pat. No. 6,476,295), improved digestibility (U.S. Pat. No. 6,531,648), low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), and biofuel production (U.S. Pat. No. 5,998,700), the genetic elements and transgenes described in the patents listed above are herein incorporated by reference. Preferred starch branching enzymes (for modification of starch properties) include those set forth in U.S. Pat. Nos. 6,232,122 and 6,147,279; and PCT Publication WO 97/22703, all of which are incorporated herein by reference.
Modified oils production (U.S. Pat. No. 6,444,876), high oil production (U.S. Pat. Nos. 5,608,149 and 6,476,295), or modified fatty acid content (U.S. Pat. No. 6,537,750). Preferred fatty acid pathway enzymes include thioesterases (U.S. Pat. Nos. 5,512,482; 5,530,186; 5,945,585; 5,639,790; 5,807,893; 5,955,650; 5,955,329; 5,759,829; 5,147,792; 5,304,481; 5,298,421; 5,344,771; and 5,760,206), diacylglycerol acyltransferases (U.S. Patent Publications 20030115632A1, 2, 3, 4, 5, 6, 7, 8, and 90030028923A1), and desaturases (U.S. Pat. Nos. 5,689,050; 5,663,068; 5,614,393; 5,856,157; 6,117,677; 6,043,411; 6,194,167; 5,705,391; 5,663,068; 5,552,306; 6,075,183; 6,051,754; 5,689,050; 5,789,220; 5,057,419; 5,654,402; 5,659,645; 6,100,091; 5,760,206; 6,172,106; 5,952,544; 5,866,789; 5,443,974; and 5,093,249) all of which are incorporated herein by reference.
Preferred amino acid biosynthetic enzymes include anthranilate synthase (U.S. Pat. No. 5,965,727 and PCT Publications WO 97/26366, WO 99/11800, WO 99/49058), tryptophan decarboxylase (PCT Publication WO 99/06581), threonine decarboxylase (U.S. Pat. Nos. 5,534,421 and 5,942,660; PCT Publication WO 95/19442), threonine deaminase (PCT Publications WO 99/02656 and WO 98/55601), dihydrodipicolinic acid synthase (U.S. Pat. No. 5,258,300), and aspartate kinase (U.S. Pat. Nos. 5,367,110; 5,858,749; and 6,040,160) all of which are incorporated herein by reference.
Production of nutraceuticals such as, for example, polyunsaturated fatty acids (for example arachidonic acid, eicosapentaenoic acid or docosahexaenoic acid) by expression of fatty acid elongases and/or desaturases, or production of proteins with improved nutritional value such as, for example, with a high content of essential amino acids (for example the high-methionine 2S albumin gene of the brazil nut). Preferred are nucleic acids which encode the Bertholletia excelsa high-methionine 2S albumin (GenBank Acc. No.: AB044391), the Physcomitrella patens Delta-6-acyl-lipid desaturase (GenBank Acc. No.: AJ222980; Girke et al. (1998) Plant J 15:39-48), the Mortierella alpina Delta-6-desaturase (Sakuradani et al. 1999 Gene 238:445-453), the Caenorhabditis elegans Delta-5-desaturase (Michaelson et al. 1998, FEBS Letters 439:215-218), the Caenorhabditis elegans Delta-5-fatty acid desaturase (des-5) (GenBank Acc. No.: AF078796), the Mortierella alpina Delta-5-desaturase (Michaelson et al. JBC 273:19055-19059), the Caenorhabditis elegans Delta-6-elongase (Beaudoin et al. 2000, PNAS 97:6421-6426), the Physcomitrella patens Delta-6-elongase (Zank et al. 2000, Biochemical Society Transactions 28:654-657), or functional equivalents of these.
Production of high-quality proteins and enzymes for industrial purposes (for example enzymes, such as lipases) or as pharmaceuticals (such as, for example, antibodies, blood clotting factors, interferons, lymphokins, colony stimulation factor, plasminogen activators, hormones or vaccines, as described by Hood E E, Jilka J M (1999) Curr Opin Biotechnol 10(4): 382-6; Ma J K, Vine N D (1999) Curr Top Microbiol Immunol 236:275-92). For example, it has been possible to produce recombinant avidin from chicken albumen and bacterial beta-glucuronidase (GUS) on a large scale in transgenic maize plants (Hood et al. (1999) Adv Exp Med Biol 464:127-47. Review).
Obtaining an increased storability in cells which normally comprise fewer storage proteins or storage lipids, with the purpose of increasing the yield of these substances, for example by expression of acetyl-CoA carboxylase. Preferred nucleic acids are those, which encode the Medicago sativa acetyl-CoA carboxylase (ACCase) (GenBank Acc. No.: L25042), or functional equivalents thereof. Alternatively, in some scenarios an increased storage protein content might be advantageous for high-protein product production. Preferred seed storage proteins include zeins (U.S. Pat. Nos. 4,886,878; 4,885,357; 5,215,912; 5,589,616; 5,508,468; 5,939,599; 5,633,436; and 5,990,384; PCT Publications WO 90/01869, WO 91/13993, WO 92/14822, WO 93/08682, WO 94/20628, WO 97/28247, WO 98/26064, and WO 99/40209), 7S proteins (U.S. Pat. Nos. 5,003,045 and 5,576,203), brazil nut protein (U.S. Pat. No. 5,850,024), phenylalanine free proteins (PCT Publication WO 96/17064), albumin (PCT Publication WO 97/35023), b-conglycinin (PCT Publication WO 00/19839), 11S (U.S. Pat. No. 6,107,051), alpha-hordothionin (U.S. Pat. Nos. 5,885,802 and 5,88,5801), arcelin seed storage proteins (U.S. Pat. No. 5,270,200), lectins (U.S. Pat. No. 6,110,891), and glutenin (U.S. Pat. Nos. 5,990,389 and 5,914,450) all of which are incorporated herein by reference.
Reducing levels of alpha-glucan L-type tuber phosphorylase (GLTP) or alpha-glucan H-type tuber phosphorylase (GHTP) enzyme activity preferably within the potato tuber (see U.S. Pat. No. 5,998,701). The conversion of starches to sugars in potato tubers, particularly when stored at temperatures below 7° C., is reduced in tubers exhibiting reduced GLTP or GHTP enzyme activity. Reducing cold-sweetening in potatoes allows for potato storage at cooler temperatures, resulting in prolonged dormancy, reduced incidence of disease, and increased storage life. Reduction of GLTP or GHTP activity within the potato tuber may be accomplished by such techniques as suppression of gene expression using homologous antisense or double-stranded RNA, the use of co-suppression, regulatory silencing sequences. A potato plant having improved cold-storage characteristics, comprising a potato plant transformed with an expression cassette having a TPT promoter sequence operably linked to a DNA sequence comprising at least 20 nucleotides of a gene encoding an alpha-glucan phosphorylase selected from the group consisting of alpha-glucan L-type tuber phosphorylase (GLTP) and alpha-glucan H-type phosphorylase (GHTP).
   Further examples of advantageous genes are mentioned for example in Dunwell J M, Transgenic approaches to crop improvement, J Exp Bot. 2000; 51 Spec No; pages 487-96.

### 1.8 Plant Agronomic Characteristics

Two of the factors determining where plants can be grown are the average daily temperature during the growing season and the length of time between frosts. Within the areas where it is possible to grow a particular plant, there are varying limitations on the maximal time it is allowed to grow to maturity and be harvested. The plant to be grown in a particular area is selected for its ability to mature and dry down to harvestable moisture content within the required period of time with maximum possible yield. Therefore, plants of varying maturities are developed for different growing locations. Apart from the need to dry down sufficiently to permit harvest is the desirability of having maximal drying take place in the field to minimize the amount of energy required for additional drying post-harvest. Also the more readily the grain can dry down, the more time there is available for growth and kernel fill. Genes that influence maturity and/or dry down can be identified and introduced into plant lines using transformation techniques to create new varieties adapted to different growing locations or the same growing location but having improved yield to moisture ratio at harvest. Expression of genes that are involved in regulation of plant development may be especially useful, e.g., the liguleless and rough sheath genes that have been identified in plants.

Genes may be introduced into plants that would improve standability and other plant growth characteristics. For example, expression of novel genes, which confer stronger stalks, improved root systems, or prevent or reduce ear droppage would be of great value to the corn farmer. Introduction and expression of genes that increase the total amount of photoassimilate available by, for example, increasing light distribution and/or interception would be advantageous. In addition the expression of genes that increase the efficiency of photosynthesis and/or the leaf canopy would further increase gains in productivity. Such approaches would allow for increased plant populations in the field.

Delay of late season vegetative senescence would increase the flow of assimilates into the grain and thus increase yield. Overexpression of genes within plants that are associated with “stay green” or the expression of any gene that delays senescence would be advantageous. For example, a non-yellowing mutant has been identified in Festuca pratensis (Davies 1990). Expression of this gene as well as others may prevent premature breakdown of chlorophyll and thus maintain canopy function.

### 1.9 Nutrient Utilization

The ability to utilize available nutrients and minerals may be a limiting factor in growth of many plants. It is proposed that it would be possible to alter nutrient uptake, tolerate pH extremes, mobilization through the plant, storage pools, and availability for metabolic activities by the introduction of novel genes. These modifications would allow a plant to more efficiently utilize available nutrients. It is contemplated that an increase in the activity of, for example, an enzyme that is normally present in the plant and involved in nutrient utilization would increase the availability of a nutrient. An example of such an enzyme would be phytase. It is also contemplated that expression of a novel gene may make a nutrient source available that was previously not accessible, e.g., an enzyme that releases a component of nutrient value from a more complex molecule, perhaps a macromolecule.

### 1.10 Male Sterility

Male sterility is useful in the production of hybrid seed. It is proposed that male sterility may be produced through expression of novel genes. For example, it has been shown that expression of genes that encode proteins that interfere with development of the male inflorescence and/or gametophyte result in male sterility. Chimeric ribonuclease genes that express in the anthers of transgenic tobacco and oilseed rape have been demonstrated to lead to male sterility (Mariani 1990). For example, a number of mutations were discovered in maize that confer cytoplasmic male sterility. One mutation in particular, referred to as T cytoplasm, also correlates with sensitivity to Southern corn leaf blight. A DNA sequence, designated TURF-13 (Levings 1990), was identified that correlates with T cytoplasm. It would be possible through the introduction of TURF-13 via transformation to separate male sterility from disease sensitivity. As it is necessary to be able to restore male fertility for breeding purposes and for grain production, it is proposed that genes encoding restoration of male fertility may also be introduced.

### 1.11. Non-Protein-Expressing Sequences

### 1.11.1 RNA-Expressing

DNA may be introduced into plants for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes.

Genes may be constructed or isolated, which when transcribed, produce antisense RNA or double-stranded RNA that is complementary to all or part(s) of a targeted messenger RNA(s). The antisense RNA reduces production of the polypeptide product of the messenger RNA. The polypeptide product may be any protein encoded by the plant genome. The aforementioned genes will be referred to as antisense genes. An antisense gene may thus be introduced into a plant by transformation methods to produce a novel transgenic plant with reduced expression of a selected protein of interest. For example, the protein may be an enzyme that catalyzes a reaction in the plant. Reduction of the enzyme activity may reduce or eliminate products of the reaction which include any enzymatically synthesized compound in the plant such as fatty acids, amino acids, carbohydrates, nucleic acids and the like. Alternatively, the protein may be a storage protein, such as a zein, or a structural protein, the decreased expression of which may lead to changes in seed amino acid composition or plant morphological changes respectively. The possibilities cited above are provided only by way of example and do not represent the full range of applications.

Expression of antisense-RNA or double-stranded RNA by one of the expression cassettes of the invention is especially preferred. Also expression of sense RNA can be employed for gene silencing (co-suppression). This RNA is preferably a non-translatable RNA. Gene regulation by double-stranded RNA (“double-stranded RNA interference”; dsRNAi) is well known in the arte and described for various organism including plants (e.g., Matzke 2000; Fire A et al 1998; WO 99/32619; WO 99/53050; WO 00/68374; WO 00/44914; WO 00/44895; WO 00/49035; WO 00/63364).

Genes may also be constructed or isolated, which when transcribed produce RNA enzymes, or ribozymes, which can act as endoribonucleases and catalyze the cleavage of RNA molecules with selected sequences. The cleavage of selected messenger RNA's can result in the reduced production of their encoded polypeptide products. These genes may be used to prepare novel transgenic plants, which possess them. The transgenic plants may possess reduced levels of polypeptides including but not limited to the polypeptides cited above that may be affected by antisense RNA.

It is also possible that genes may be introduced to produce novel transgenic plants, which have reduced expression of a native gene product, by a mechanism of cosuppression. It has been demonstrated in tobacco, tomato, and petunia (Goring 1991; Smith 1990; Napoli 1990; van der Krol 1990) that expression of the sense transcript of a native gene will reduce or eliminate expression of the native gene in a manner similar to that observed for antisense genes. The introduced gene may encode all or part of the targeted native protein but its translation may not be required for reduction of levels of that native protein.

### 1.11.2 Non-RNA-Expressing

For example, DNA elements including those of transposable elements such as Ds, Ac, or Mu, may be, inserted into a gene and cause mutations. These DNA elements may be inserted in order to inactivate (or activate) a gene and thereby “tag” a particular trait. In this instance the transposable element does not cause instability of the tagged mutation, because the utility of the element does not depend on its ability to move in the genome. Once a desired trait is tagged, the introduced DNA sequence may be used to clone the corresponding gene, e.g., using the introduced DNA sequence as a PCR primer together with PCR gene cloning techniques (Shapiro, 1983; Dellaporta 1988). Once identified, the entire gene(s) for the particular trait, including control or regulatory regions where desired may be isolated, cloned and manipulated as desired. The utility of DNA elements introduced into an organism for purposed of gene tagging is independent of the DNA sequence and does not depend on any biological activity of the DNA sequence, i.e., transcription into RNA or translation into protein. The sole function of the DNA element is to disrupt the DNA sequence of a gene.

It is contemplated that unexpressed DNA sequences, including novel synthetic sequences could be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

Another possible element, which may be introduced, is a matrix attachment region element (MAR), such as the chicken lysozyme A element (Stief 1989), which can be positioned around an expressible gene of interest to effect an increase in overall expression of the gene and diminish position dependant effects upon incorporation into the plant genome (Stief 1989; Phi-Van 1990).

Further nucleotide sequences of interest that may be contemplated for use within the scope of the present invention in operable linkage with the promoter sequences according to the invention are isolated nucleic acid molecules, e.g., DNA or RNA, comprising a plant nucleotide sequence according to the invention comprising an open reading frame that is preferentially expressed in a specific tissue, i.e., seed-, root, green tissue (leaf and stem), panicle-, or pollen, or is expressed constitutively.

### 2. Marker Genes

In order to improve the ability to identify transformants, one may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait, the green fluorescent protein (GFP)). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers, which encode a secretable antigen that can be identified by antibody interaction, or even secretable enzymes, which can be detected by their catalytic activity. Secretable proteins fall into a number of classes, including small, diffusible proteins detectable, e.g., by ELISA; small active enzymes detectable in extracellular solution (e.g., alpha-amylase, beta-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). For example, the maize HPRG (Steifel 1990) molecule is well characterized in terms of molecular biology, expression and protein structure. However, any one of a variety of ultilane and/or glycine-rich wall proteins (Keller 1989) could be modified by the addition of an antigenic site to create a screenable marker.

One exemplary embodiment of a secretable screenable marker concerns the use of a maize sequence encoding the wall protein HPRG, modified to include a 15 residue epitope from the pro-region of murine interleukin, however, virtually any detectable epitope may be employed in such embodiments, as selected from the extremely wide variety of antigen-antibody combinations known to those of skill in the art. The unique extracellular epitope can then be straightforwardly detected using antibody labeling in conjunction with chromogenic or fluorescent adjuncts. Elements of the present disclosure may be exemplified in detail through the use of the bar and/or GUS genes, and also through the use of various other markers. Of course, in light of this disclosure, numerous other possible selectable and/or screenable marker genes will be apparent to those of skill in the art in addition to the one set forth herein below. Therefore, it will be understood that the following discussion is exemplary rather than exhaustive. In light of the techniques disclosed herein and the general recombinant techniques which are known in the art, the present invention renders possible the introduction of any gene, including marker genes, into a recipient cell to generate a transformed plant.

### 2.1 Selectable Markers

Various selectable markers are known in the art suitable for plant transformation. Such markers may include but are not limited to:

### 2.1.1 Negative Selection Markers

Negative selection markers confer a resistance to a biocidal compound such as a metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Transformed plant material (e.g., cells, tissues or plantlets), which express marker genes, are capable of developing in the presence of concentrations of a corresponding selection compound (e.g., antibiotic or herbicide), which suppresses growth of an untransformed wild type tissue. Especially preferred negative selection markers are those, which confer resistance to herbicides. Examples, which may be mentioned, are:
Phosphinothricin acetyltransferases (PAT; also named Bialophos® resistance; bar; de Block 1987; Vasil 1992, 1993; Weeks 1993; Becker 1994; Nehra 1994; Wan & Lemaux 1994; EP 0 333 033; U.S. Pat. No. 4,975,374). Preferred are the bar gene from Streptomyces hygroscopicus or the pat gene from Streptomyces viridochromogenes. PAT inactivates the active ingredient in the herbicide bialaphos, phosphinothricin (PPT). PPT inhibits glutamine synthetase, (Murakami 1986; Twell 1989) causing rapid accumulation of ammonia and cell death.
altered 5-enolpyruvylshikimate-3-phosphate synthase (EPSPS) conferring resistance to Glyphosate® (N-(phosphonomethyl)glycine) (Hinchee 1988; Shah 1986; Della-Cioppa 1987). Where a mutant EPSP synthase gene is employed, additional benefit may be realized through the incorporation of a suitable chloroplast transit peptide, CTP (EP-A10 218 571).
Glyphosate® degrading enzymes (Glyphosate® oxidoreductase; gox),
Dalapon® inactivating dehalogenases (deh)
sulfonylurea- and/or imidazolinone-inactivating acetolactate synthases (ahas or ALS; for example mutated ahas/ALS variants with, for example, the S4, XI12, XA17, and/or Hra mutation (EP-A1 154 204)
Bromoxynil® degrading nitrilases (bxn; Stalker 1988)
Kanamycin- or geneticin (G418) resistance genes (NPTII; NPT or neo; Potrykus 1985) coding e.g., for neomycin phosphotransferases (Fraley 1983; Nehra 1994)
2-Desoxyglucose-6-phosphate phosphatase (DOG^{R}1-Gene product; WO 98/45456; EP 0 807 836) conferring resistance against 2-desoxyglucose (Randez-Gil 1995).
hygromycin phosphotransferase (HPT), which mediates resistance to hygromycin (Vanden Elzen 1985).
altered dihydrofolate reductase (Eichholtz 1987) conferring resistance against methotrexat (Thillet 1988);
mutated anthranilate synthase genes that confers resistance to 5-methyl tryptophan.

Additional negative selectable marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Hayford 1988; Jones 1987; Svab 1990; Hille 1986).

Especially preferred are negative selection markers that confer resistance against the toxic effects imposed by D-amino acids like e.g., D-alanine and D-serine (WO 03/060133; Erikson 2004). Especially preferred as negative selection marker in this contest are the daol gene (EC: 1.4. 3.3: GenBank Acc.-No.: U60066) from the yeast Rhodotorula gracilis (Rhodosporidium toruloides) and the E. coli gene dsdA (D-serine dehydratase (D-serine deaminase) [EC: 4.3. 1.18; GenBank Acc.-No.: J01603).

Transformed plant material (e.g., cells, embryos, tissues or plantlets) which express such marker genes are capable of developing in the presence of concentrations of a corresponding selection compound (e.g., antibiotic or herbicide) which suppresses growth of an untransformed wild type tissue. The resulting plants can be bred and hybridized in the customary fashion. Two or more generations should be grown in order to ensure that the genomic integration is stable and hereditary. Corresponding methods are described (Jenes 1993; Potrykus 1991).

Furthermore, reporter genes can be employed to allow visual screening, which may or may not (depending on the type of reporter gene) require supplementation with a substrate as a selection compound.

Various time schemes can be employed for the various negative selection marker genes. In case of resistance genes (e.g., against herbicides or D-amino acids) selection is preferably applied throughout callus induction phase for about 4 weeks and beyond at least 4 weeks into regeneration. Such a selection scheme can be applied for all selection regimes. It is furthermore possible (although not explicitly preferred) to remain the selection also throughout the entire regeneration scheme including rooting.

For example, with the phosphinotricin resistance gene (bar) as the selective marker, phosphinotricin at a concentration of from about 1 to 50 mg/l may be included in the medium. For example, with the daol gene as the selective marker, D-serine or D-alanine at a concentration of from about 3 to 100 mg/l may be included in the medium. Typical concentrations for selection are 20 to 40 mg/l. For example, with the mutated ahas genes as the selective marker, PURSUIT™ at a concentration of from about 3 to 100 mg/l may be included in the medium. Typical concentrations for selection are 20 to 40 mg/l.

### 2.1.2 Positive Selection Marker

Furthermore, positive selection marker can be employed. Genes like isopentenyltransferase from Agrobacterium tumefaciens (strain:PO22; Genbank Acc.-No.: AB025109) may—as a key enzyme of the cytokinin biosynthesis—facilitate regeneration of transformed plants (e.g., by selection on cytokinin-free medium). Corresponding selection methods are described (Ebinuma 2000a,b). Additional positive selection markers, which confer a growth advantage to a transformed plant in comparison with a non-transformed one, are described e.g., in EP-A 0 601 092. Growth stimulation selection markers may include (but shall not be limited to) beta-Glucuronidase (in combination with e.g., a cytokinin glucuronide), mannose-6-phosphate isomerase (in combination with mannose), UDP-galactose-4-epimerase (in combination with e.g., galactose), wherein mannose-6-phosphate isomerase in combination with mannose is especially preferred.

### 2.1.3 Counter-Selection Marker

Counter-selection markers are especially suitable to select organisms with defined deleted sequences comprising said marker (Koprek 1999). Examples for counter-selection marker comprise thymidin kinases (TK), cytosine deaminases (Gleave 1999; Perera 1993; Stougaard 1993), cytochrom P450 proteins (Koprek 1999), haloalkan dehalogenases (Naested 1999), iaaH gene products (Sundaresan 1995), cytosine deaminase codA (Schlaman & Hooykaas 1997), tms2 gene products (Fedoroff & Smith 1993), or alpha-naphthalene acetamide (NAM; Depicker 1988). Counter selection markers may be useful in the construction of transposon tagging lines. For example, by marking an autonomous transposable element such as Ac, Master Mu, or En/Spn with a counter selection marker, one could select for transformants in which the autonomous element is not stably integrated into the genome. This would be desirable, for example, when transient expression of the autonomous element is desired to activate in trans the transposition of a defective transposable element, such as Ds, but stable integration of the autonomous element is not desired. The presence of the autonomous element may not be desired in order to stabilize the defective element, i.e., prevent it from further transposing. However, it is proposed that if stable integration of an autonomous transposable element is desired in a plant the presence of a negative selectable marker may make it possible to eliminate the autonomous element during the breeding process.

### 2.2. Screenable Markers

Screenable markers that may be employed include, but are not limited to, a beta-glucuronidase (GUS) or uidA gene which encodes an enzyme for which various chromogenic substrates are known; an R-locus gene, which encodes a product that regulates the production of anthocyanin pigments (red color) in plant tissues (Dellaporta 1988); a beta-lactamase gene (Sutcliffe 1978), which encodes an enzyme for which various chromogenic substrates are known (e.g., PADAC, a chromogenic cephalosporin); a xyIE gene (Zukowsky 1983) which encodes a catechol dioxygenase that can convert chromogenic catechols; an alpha-amylase gene (Ikuta 1990); a tyrosinase gene (Katz 1983) which encodes an enzyme capable of oxidizing tyrosine to DOPA and dopaquinone which in turn condenses to form the easily detectable compound melanin; beta-galactosidase gene, which encodes an enzyme for which there are chromogenic substrates; a luciferase (lux) gene (Ow 1986), which allows for bioluminescence detection; or even an aequorin gene (Prasher 1985), which may be employed in calcium-sensitive bioluminescence detection, or a green fluorescent protein gene (Niedz 1995).

Genes from the maize R gene complex are contemplated to be particularly useful as screenable markers. The R gene complex in maize encodes a protein that acts to regulate the production of anthocyanin pigments in most seed and plant tissue. A gene from the R gene complex was applied to maize transformation, because the expression of this gene in transformed cells does not harm the cells. Thus, an R gene introduced into such cells will cause the expression of a red pigment and, if stably incorporated, can be visually scored as a red sector. If a maize line is dominant for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together.

It is further proposed that R gene regulatory regions may be employed in chimeric constructs in order to provide mechanisms for controlling the expression of chimeric genes. More diversity of phenotypic expression is known at the R locus than at any other locus (Coe 1988). It is contemplated that regulatory regions obtained from regions 5′ to the structural R gene would be valuable in directing the expression of genes, e.g., insect resistance, drought resistance, herbicide tolerance or other protein coding regions. For the purposes of the present invention, it is believed that any of the various R gene family members may be successfully employed (e.g., P, S, Lc, etc.). However, the most preferred will generally be Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for populational screening for bioluminescence, such as on tissue culture plates, or even for whole plant screening. Where use of a screenable marker gene such as lux or GFP is desired, benefit may be realized by creating a gene fusion between the screenable marker gene and a selectable marker gene, for example, a GFP-NPTII gene fusion. This could allow, for example, selection of transformed cells followed by screening of transgenic plants or seeds.

### 3. Uses of Transgenic Plants

Once an expression cassette of the invention has been transformed into a particular plant species, it may be propagated in that species or moved into other varieties of the same species, particularly including commercial varieties, using traditional breeding techniques. Particularly preferred plants of the invention include the agronomically important crops listed above. The genetic properties engineered into the transgenic seeds and plants described above are passed on by sexual reproduction and can thus be maintained and propagated in progeny plants. The present invention also relates to a transgenic plant cell, tissue, organ, seed or plant part obtained from the transgenic plant. Also included within the invention are transgenic descendants of the plant as well as transgenic plant cells, tissues, organs, seeds and plant parts obtained from the descendants.

Preferably, the expression cassette in the transgenic plant is sexually transmitted. In one preferred embodiment, the coding sequence is sexually transmitted through a complete normal sexual cycle of the R0 plant to the R1 generation. Additionally preferred, the expression cassette is expressed in the cells, tissues, seeds or plant of a transgenic plant in an amount that is different than the amount in the cells, tissues, seeds or plant of a plant, which only differs in that the expression cassette is absent.

The transgenic plants produced herein are thus expected to be useful for a variety of commercial and research purposes. Transgenic plants can be created for use in traditional agriculture to possess traits beneficial to the grower (e.g., agronomic traits such as resistance to water deficit, pest resistance, herbicide resistance or increased yield), beneficial to the consumer of the grain harvested from the plant (e.g., improved nutritive content in human food or animal feed; increased vitamin, amino acid, and antioxidant content; the production of antibodies (passive immunization) and nutriceuticals), or beneficial to the food processor (e.g., improved processing traits). In such uses, the plants are generally grown for the use of their grain in human or animal foods. Additionally, the use of root-specific promoters in transgenic plants can provide beneficial traits that are localized in the consumable (by animals and humans) roots of plants such as carrots, parsnips, and beets. However, other parts of the plants, including stalks, husks, vegetative parts, and the like, may also have utility, including use as part of animal silage or for ornamental purposes. Often, chemical constituents (e.g., oils or starches) of maize and other crops are extracted for foods or industrial use and transgenic plants may be created which have enhanced or modified levels of such components.

Transgenic plants may also find use in the commercial manufacture of proteins or other molecules, where the molecule of interest is extracted or purified from plant parts, seeds, and the like. Cells or tissue from the plants may also be cultured, grown in vitro, or fermented to manufacture such molecules. The transgenic plants may also be used in commercial breeding programs, or may be crossed or bred to plants of related crop species. Improvements encoded by the expression cassette may be transferred, e.g., from maize cells to cells of other species, e.g., by protoplast fusion.

The transgenic plants may have many uses in research or breeding, including creation of new mutant plants through insertional mutagenesis, in order to identify beneficial mutants that might later be created by traditional mutation and selection. An example would be the introduction of a recombinant DNA sequence encoding a transposable element that may be used for generating genetic variation. The methods of the invention may also be used to create plants having unique “signature sequences” or other marker sequences which can be used to identify proprietary lines or varieties.

Thus, the transgenic plants and seeds according to the invention can be used in plant breeding, which aims at the development of plants with improved properties conferred by the expression cassette, such as tolerance of drought, disease, or other stresses. The various breeding steps are characterized by well-defined human intervention such as selecting the lines to be crossed, directing pollination of the parental lines, or selecting appropriate descendant plants. Depending on the desired properties different breeding measures are taken. The relevant techniques are well known in the art and include but are not limited to hybridization, inbreeding, backcross breeding, multilane breeding, variety blend, interspecific hybridization, aneuploid techniques, etc. Hybridization techniques also include the sterilization of plants to yield male or female sterile plants by mechanical, chemical or biochemical means. Cross-pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines, which for example increase the effectiveness of conventional methods such as herbicide or pesticide treatment or allow dispensing with said methods due to their modified genetic properties. Alternatively new crops with improved stress tolerance can be obtained which, due to their optimized genetic “equipment”, yield harvested product of better quality than products, which were not able to tolerate comparable adverse developmental conditions. The invention will be further illustrated by the following examples.

### EXAMPLES

### Example 1

### Identification of KG (Keygene) Transcript Candidates

A maize gene expression profiling analysis was carried out using a commercial supplier of AFLP comparative expression technology (Keygene N.V., P.O. Box 216, 6700 AE Wageningen, The Netherlands) using a battery of RNA samples from 23 maize tissues generated by the inventors of the present invention (Table 1). Nine fragments were identified as having embryo or whole seed specific expression. These fragments were designated as KG_Fragment 56, 129, 49, 24, 37, 45, 46, 103, 119, respectively. Sequences of each fragment are shown in SEQ ID NOs: 145 to 153.

TABLE 1
Corn Tissues used for mRNA expression profiling experiment

| Sample | | Timing and | Days after |
|---|---|---|---|
| No. | Tissue | number of plants | Pollination |
| 1 | Root | 9 am (4 plants) | 5 |
| 2 | | 9 am (4 plants) | 15 |
| 3 | | 9 am (4 plants) | 30 |
| 4 | leaf above the ear | 9 am (6 plants) | 5 |
| 5 | | 9 am (6 plants) | 15 |
| 6 | | 9 am (6 plants) | 30 |
| 7 | ear complete | 9 am (6 plants) | 5 |
| 8 | | 9 am (6 plants) | 10 |
| 9 | Whole seed | 9 am (6 plants) | 15 |
| 10 | | 9 am (6 plants) | 20 |
| 11 | | 9 am (6 plants) | 30 |
| 12 | Endosperm | 9 am (6 plants) | 15 |
| 13 | | 9 am (6 plants) | 20 |
| 14 | | 9 am (6 plants) | 30 |
| 15 | Embryo | 9 am (6 plants) | 15 |
| 16 | | 9 am (6 plants) | 20 |
| 17 | | 9 am (6 plants) | 30 |
| 18 | Female pistilate flower | 6 plants | before |
| | | | pollination |
| 19 | germinating seed | 20 seeds | imbibition |
| | | | for 3 days |
| 20 | root, veg. state | | V2 |
| 21 | root, veg. state | | V4 |
| 22 | leaf, veg. State | | V2 |
| 23 | leaf, veg. State | | V4 |

### Example 2

### Identification of the EST Corresponding to KG_Fragment Candidates

Sequences of the KG_Fragment candidates were used as query for BLASTN searching against inventor's in-house database, HySeq All EST. EST accessions showing highest identities to above KG_Fragments are listed in Table 2 and sequences of these ESTs are shown in SEQ ID NOs: 93, 94, and 98-104.

TABLE 2
Maize EST accession number showing highest
identities to the KG fragment candidates

| KG | | | SEQ |
|---|---|---|---|
| Fragment ID | Hyseq Maize EST ID | % identities | ID NO: |
| 24 | 62001211.f01 | 100 | 99 |
| 37 | 62029487.f01 | 100 | 100 |
| 45 | 57894155.f01 | 100 | 101 |
| 46 | 62096689.f01 | 100 | 102 |
| 49 | 62158447.f01 | 91 | 98 |
| 56 | no | N/A | 93 |
| 103 | ZM07MC01323_57619299 | 100 | 103 |
| 119 | ZM07MC15086_59463108 | 100 | 104 |
| 129 | 62092959.f01 | 100 | 94 |

### Example 3

### Confirmation of Expression Pattern of the KG Candidates Using Quantitative Reverse Transcriptase-Polymerase Chain Reaction (Q-RT-PCR)

In order to confirm the native expression pattern of the KG candidates, quantitative reverse transcription PCR (q-RT-PCR) was performed using total RNA isolated from the same materials as were used for the AFLP expression profiling (Table 1).

Primers for qRT-PCR were designed based on the sequences of either the KG_Fragments or the identified maize Hyseq EST using the Vector NTI software package (Invitrogen, Carlsbad, Calif., USA). Two sets of primers were used for PCR amplification for each candidate. The glyceraldehyde-3-phosphate dehydrogenase (GAPDH) gene served as a control for normalization purposes. Sequences of primers for q-RT-PCR are listed in Table 3.

TABLE 3
Primer sequences for q-RT-PCR

| Primer | Sequences |
|---|---|
| KG24_forward_1 | GTGGCTGTCATACTGGAT |
| KG24_reverse_1 | GAGCTTCTCGTAGACGAA |
| KG24_forward_2 | TCACAGGAACTTCTGTAGAT |
| KG24_reverse_2 | TCGTTCTTACAGAAGCAT |
| KG37_forward_1 | AAGGCATGTTATGCTCGA |
| KG37_reverse_1 | AAACTCGAAAACCGCCAC |
| KG37_forward_2 | AGGCAAGTTCAAGACAAC |
| KG37_reverse_2 | AAAAATCCCATCTGTCCC |
| KG45_forward_1 | TGCTGGTGAATGATGGTT |
| KG45_reverse_1 | CACATCGTTCGCTACATA |
| KG45_forward_2 | ACGCCTCCCCTCGTGATT |
| KG45_reverse_2 | TGCCAGACGTACCCGACGG |
| KG46_forward_1 | CTGCGGAGGCGAACAGGA |
| KG46_reverse_1 | GCTTGTCGACGGAGACGG |
| KG46_forward_2 | CCGGACATCGGCGTCTACCTC |
| KG46_reverse_2 | CCGTTCGGGAACACCACC |
| KG49_forward_1 | CAGCTGGTGGGGAGGATAT |
| KG49_reverse_1 | CGAGCCTGTGAATTGCAT |
| KG49_forward_2 | ATCTTCTCACGATCCAGG |
| KG49_reverse_2 | TTGTGAACAGCATGTCCC |
| KG56_forward_1 | AAATACGAAGCCCGGATC |
| KG56_reverse_1 | TAGTGTCCGTCCACCTGT |
| KG56_forward_2 | AGCCAGGGCCATATAACA |
| KG56_reverse_2 | TAGCTGTTTCTGCCCATA |
| KG103_forward_1 | TCCACCTTAGCCTAGGGTT |
| KG103_reverse_1 | AACACGCAGCTTTCCAAA |
| KG103_forward_2 | CAAGCTCTCCCTGGAGAT |
| KG103_reverse_2 | GCGAAGACCACACAGACA |
| KG119_forward_1 | CAGACAGACCACTGACTGCAT |
| KG119_reverse_1 | GTTAGGCCTGTGCGTGTG |
| KG119_forward_2 | CTGAGAGCCCCGGAACTCGTT |
| KG119_reverse_2 | TGTGCCGGGCTCTGGGTT |
| KG129_forward_1 | GCTCACCAACGGAGTGAT |
| KG129_reverse_1 | CATCAGAGTTCCCGTCGT |
| KG129_forward_2 | GTCTCTCCCCGCTAGTGACTT |
| KG129_reverse_2 | GGGAAAGTCGCTCACGAA |
| GAPDH_Forward | GTAAAGTTCTTCCTGATCTGAAT |
| GAPDH_Reverse | TCGGAAGCAGCCTTAATA |

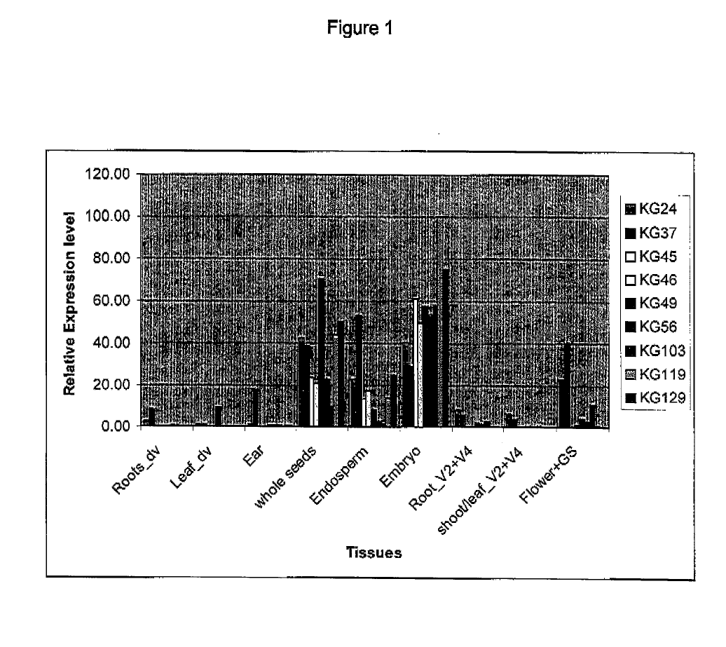

q-RT-PCR was performed using SuperScript III Reverse Transcriptase (Invitrogen, Carlsbad, Calif., USA) and SYBR Green QPCR Master Mix (Eurogentec, San Diego, Calif., USA) in an ABI Prism 7000 sequence detection system. In brief, cDNA was synthesized using 2-3 microgram of total RNA and 1 μL reverse transcriptase in a 20 μl volume. The cDNA was diluted to a range of concentrations (15-20 ng/μl). Thirty to forty ng of cDNA was used for quantitative PCR (qPCR) in a 30 μL volume with SYBR Green QPCR Master Mix following the manufacturer's instruction. The thermocycling conditions were as follows: incubate at 50° C. for 2 minutes, denature at 95° C. for 10 minutes, and run 40 cycles at 95° C. for 15 seconds and 60° C. for 1 minute for amplification. After the final cycle of the amplification, the dissociation curve analysis was carried out to verify that the amplification occurred specifically and no primer dimer product was generated during the amplification process. The housekeeping gene glyceraldehyde-3-phosphate-dehydrogenase (GAPDH, primer sequences in Table 3) was used as an endogenous reference gene to normalize the calculation using the Comparative Ct (Cycle of threshold) value method. The ACT value was obtained by subtracting the Ct value of GAPDH gene from the Ct value of the candidate gene, and the relative transcription quantity (expression level) of the candidate gene was expressed as 2^{-ΔCT}. The q-RT-PCR results are summarized in FIG. 1. All KG candidates showed similar expression patterns that are equivalent to the expression patterns obtained from the AFLP data: specifically or preferably expressed in embryo or whole seeds (FIG. 1).

### Example 4

### Annotation and Promoter Identification of the KG Candidates

The coding sequences corresponding to KG candidates were annotated based on the in silico results obtained from both BLASTX of each EST sequence against GenBank protein database (nr) and the result of in silico translation of the sequence using Vector NTI software package.

### 1). KG_fragment 24

Maize EST 62001211.f01 encodes a protein that has homology to a hypothetical protein of wheat (GenBank Accession: BAC80265). The top 10 homologous sequences identified in the BlastX query are presented in Table 4.

TABLE 4
BLASTX search results of KG_{—}
fragment 24/Hyseq EST 62001211.f01

| | | | | % Iden- |
|---|---|---|---|---|
| Accession | Description | Score | E-value | tities |
| BAC80265 | hypothetical protein | 191 | 9.00E−56 | 81 |
| | [Triticum aestivum]. | | | |
| Q07764 | HVA22_HORVU | 191 | 2.00E−54 | 81 |
| | Protein HVA22 | | | |
| EAY81013.1 | hypothetical protein | 162 | 2.00E−39 | 67 |
| | OsJ_OsI_034972 | | | |
| | [Oryza sativa | | | |
| | (indica cultivar-group)] | | | |
| NP_001062004.1 | Os08g0467500 [Oryza | 133 | 5.00E−39 | 76 |
| | sativa (japonica | | | |
| | cultivar-group)] | | | |
| EAZ18437.1 | hypothetical protein | 161 | 6.00E−39 | 66 |
| | OsJ_032646 [Oryza | | | |
| | sativa (japonica | | | |
| | cultivar-group)] | | | |
| NP_001067939.1 | Os11g0498600 [Oryza | 161 | 6.00E−39 | 66 |
| | sativa (japonica | | | |
| | cultivar-group)] | | | |
| NP_568744.1 | ATHVA22E | 148 | 1.00E−35 | 62 |
| | (Arabidopsis thaliana | | | |
| | HVA22 homologue E) | | | |
| BAD09552.1 | putative abscisic | 119 | 5.00E−35 | 75 |
| | acid-induced protein | | | |
| | [Oryza sativa | | | |
| | Japonica Group] | | | |
| NP_567713.1 | ATHVA22D | 139 | 1.00E−31 | 57 |
| | (Arabidopsis thaliana | | | |
| | HVA22 homologue D) | | | |
| EAZ07285.1 | hypothetical protein | 119 | 1.00E−31 | 75 |
| | OsI_028517 [Oryza | | | |
| | sativa (indica | | | |
| | cultivar-group)] | | | |

The CDS sequence of the gene corresponding to KG_Fragment 24 is shown in SEQ ID NO: 27 and the translated amino acid sequence is shown in SEQ ID NO: 45

### Identification of the Promoter Region of KG24

For our promoter identification purposes, the sequence upstream of the start codon of the predicted KG_Fragment 24 gene was defined as the promoter p-KG24. To identify this predicted promoter region, the EST sequence of 62001211.f_o1 was mapped to the BASF Plant Science proprietary maize genomic DNA sequence database, PUB_tigr_maize_genomic_partial_{—}5.0.nt. One maize genomic DNA sequence, AZM5_{—}23949 (3602 bp) was identified (SEQ ID NO: 81). This 3602 bp sequence harbored the predicted CDS of the corresponding gene to KG_Fragment 24 and more than 1.6 kb upstream sequence of the ATG start codon of this gene

### Isolation of the Promoter Region of KG24 by PCR Amplification

The putative promoter region was isolated via genomic PCR using the following sequence specific primers:

Forward primer:

(SEQ ID NO: 154)

CTACATTTATGTTATAGAGGCGCAA
Reverse primer:

(SEQ ID NO: 155)

CATCTCTTGGGACGGAACCAA.

The expected 1507 bp fragment was amplified from maize genomic DNA, and named as promoter KG24 (p-KG24). Sequence of p-KG24 is shown in SEQ ID NO: 9.
BLASTN Results of p_KG24

The top 13 homologous sequences identified in the BlastN query are presented in Table 5.

TABLE 5
BlastN results of p_KG24

| | | Max | Total | Query | | Max |
|---|---|---|---|---|---|---|
| Accession | Description | score | score | coverage | E value | ident |
| AF205807.1 | Zea mays subsp. | 522 | 660 | 22% | 2.00E−144 | 94% |
| | huehuetenangensis | | | | | |
| | isolate Beadle s.n. b1 | | | | | |
| | gene, B-M033 allele, | | | | | |
| | partial sequence | | | | | |
| EU961185.1 | Zea mays clone | 491 | 491 | 21% | 3.00E−135 | 93% |
| | 233237 unknown | | | | | |
| | mRNA | | | | | |
| EU945925.1 | Zea mays clone | 491 | 491 | 21% | 3.00E−135 | 93% |
| | 290258 mRNA | | | | | |
| | sequence | | | | | |
| AF448416.1 | Zea mays B73 | 491 | 660 | 33% | 3.00E−135 | 94% |
| | chromosome 9S bz | | | | | |
| | genomic region | | | | | |
| AC157319.2 | Zea mays clone | 489 | 489 | 21% | 1.00E−134 | 93% |
| | ZMMBBb-136E2, | | | | | |
| | complete sequence | | | | | |
| AY883458.1 | Zea mays subsp. | 484 | 484 | 21% | 5.00E−133 | 92% |
| | parviglumis cultivar | | | | | |
| | CIMMYT-11355 | | | | | |
| | teosinte glume | | | | | |
| | architecture 1 (tga1) | | | | | |
| | gene, promoter region | | | | | |
| AY508163.1 | Zea mays cultivar | 479 | 479 | 21% | 2.00E−131 | 92% |
| | F324 disrupted | | | | | |
| | peroxidase (pox3) | | | | | |
| | gene, exons 1 through | | | | | |
| | 3; and transposon | | | | | |
| | MITE, complete | | | | | |
| | sequence | | | | | |
| AY508162.1 | Zea mays cultivar | 479 | 479 | 21% | 2.00E−131 | 92% |
| | F227 disrupted | | | | | |
| | peroxidase (pox3) | | | | | |
| | gene, exons 1 through | | | | | |
| | 3; and transposon | | | | | |
| | MITE, complete | | | | | |
| | sequence | | | | | |
| AY508161.1 | Zea mays cultivar | 479 | 479 | 21% | 2.00E−131 | 92% |
| | F226 disrupted | | | | | |
| | peroxidase (pox3) | | | | | |
| | gene, exons 1 through | | | | | |
| | 3; and transposon | | | | | |
| | MITE, complete | | | | | |
| | sequence | | | | | |
| AY508160.1 | Zea mays cultivar | 479 | 479 | 21% | 2.00E−131 | 92% |
| | F7012 disrupted | | | | | |
| | peroxidase (pox3) | | | | | |
| | gene, exons 1 through | | | | | |
| | 3; and transposon | | | | | |
| | MITE, complete | | | | | |
| | sequence | | | | | |
| AY508159.1 | Zea mays cultivar | 479 | 479 | 21% | 2.00E−131 | 92% |
| | Quebec28 disrupted | | | | | |
| | peroxidase (pox3) | | | | | |
| | gene, pox3-2 allele, | | | | | |
| | exons 1 through 3; and | | | | | |
| | transposon MITE, | | | | | |
| | complete sequence | | | | | |
| AY883461.1 | Zea mays subsp. | 479 | 479 | 21% | 2.00E−131 | 93% |
| | parviglumis cultivar | | | | | |
| | HGW-Wilkes Site 6 | | | | | |
| | teosinte glume | | | | | |
| | architecture 1 (tga1) | | | | | |
| | gene, promoter region | | | | | |
| AY508516.1 | Zea mays disrupted | 475 | 475 | 21% | 3.00E−130 | 92% |
| | peroxidase (pox3) | | | | | |
| | gene, partial | | | | | |
| | sequence; and | | | | | |
| | transposon MITE, | | | | | |
| | complete sequence | | | | | |

### 2). KG_fragment 37

KG_fragment 37/Maize EST 62029487.f01 encodes a protein that is homologous to a hypothetical protein of rice (GenBank Accession: NP_{—}001051496). The top 15 homologous sequences identified in the BlastX query are presented in Table 6.

TABLE 6
BLASTX search results of KG_{—}
fragment 37/Hyseq EST 62029487.f01

| | | | | % Iden- |
|---|---|---|---|---|
| Accession | Description | Score | E-value | tities |
| NP_001051496 | Os03g0787200 [Oryza | 518 | e−145 | 65 |
| | sativa (japonica | | | |
| | cultivar-group)]. | | | |
| EAY92106 | hypothetical protein | 518 | e−145 | 65 |
| | OsI_013339 [Oryza | | | |
| | sativa (indica | | | |
| | cultivar-group) | | | |
| ABF99245 | IQ calmodulin-binding | 479 | e−133 | 67 |
| | motif family protein, | | | |
| | expressed [Oryza | | | |
| | sativa(japonica | | | |
| | cultivar-group)] | | | |
| CAO70668 | unnamed protein | 367 | 2.00E−99 | 51 |
| | product [Vitis vinifera] | | | |
| CAN68445 | hypothetical protein | 318 | 8.00E−85 | 46 |
| | [Vitis vinifera]. | | | |
| NP_001067295 | Os12g0619000 [Oryza | 301 | 1.00E−79 | 45 |
| | sativa (japonica | | | |
| | cultivar-group)] | | | |
| NP_188858 | IQD5 (IQ-domain 5); | 301 | 2.00E−79 | 49 |
| | calmodulin binding | | | |
| | [Arabidopsis thaliana] | | | |
| BAB03067 | unnamed protein | 298 | 1.00E−78 | 44 |
| | product | | | |
| | [Arabidopsis thaliana] | | | |
| ACF85687 | unknown [Zea mays] | 294 | 2.00E−77 | 45 |
| EAY94673 | hypothetical protein | 280 | 4.00E−73 | 43 |
| | OsI_015906 [Oryza | | | |
| | sativa (indica | | | |
| | cultivar-group)] | | | |
| EAY83925 | hypothetical protein | 279 | 5.00E−73 | 43 |
| | OsI_037884 [Oryza | | | |
| | sativa (indica | | | |
| | cultivar-group)] | | | |
| NP_001050778 | Os03g0648300 [Oryza | 276 | 6.00E−72 | 43 |
| | sativa (japonica | | | |
| | cultivar-group)] | | | |
| AAU89191 | expressed protein | 276 | 6.00E−72 | 43 |
| | [Oryza sativa | | | |
| | (japonica | | | |
| | cultivar-group)] | | | |
| EAZ27950 | hypothetical protein | 269 | 3.00E−27 | 44 |
| | OsJ_011433 [Oryza | | | |
| | sativa (japonica | | | |
| | cultivar-group)] | | | |
| EAY92104 | hypothetical protein | 266 | 4.00E−69 | 73 |
| | OsI_013337 [Oryza | | | |
| | sativa (indica | | | |
| | cultivar-group)] | | | |

The CDS sequence of the gene corresponding to KG_Fragment 37 is shown in SEQ ID NO: 28 and the translated amino acid sequence is shown in SEQ ID NO: 46.

Identification of the promoter region of KG37

For our promoter identification purposes, the sequence upstream of the start codon of the predicted KG_Fragment 37 gene was defined as the promoter p-KG37. To identify this predicted promoter region, the EST sequence of 62029487.f_o1 was mapped to the BASF Plant Science proprietary maize genomic DNA sequence database, PUB_tigr_maize_genomic_partial_{—}5.0.nt. The reverse complement sequence of AZM5_{—}22959 (2441 bp) was identified (SEQ ID NO: 82). This 2441 bp sequence harbored partial predicted CDS of the corresponding gene to KG_Fragment 37 and about 1.4 kb upstream sequence of the ATG start codon of this gene.

### Isolation of the Promoter Region of KG37 by PCR Amplification

The putative promoter region was isolated via genomic PCR using the following sequence specific primers:

Forward primer:

(SEQ ID NO: 156)

CATACGATTTCCTAAGCGGAATC
Reverse primer:

(SEQ ID NO: 157)

CCGCCCGCCTCAACCACAGT.

The expected 910 bp fragment was amplified from maize genomic DNA, and named as promoter KG37 (p-KG37). Sequence of p-KG37 is shown in SEQ ID NO: 10.
BLASTN results of p_KG37

The top 11 homologous sequences identified in the BlastN query are presented in Table 7.

TABLE 7
BlastN results of p_KG37

| | | Max | Total | Query | | Max |
|---|---|---|---|---|---|---|
| Accession | Description | score | score | coverage | E value | ident |
| EU966853.1 | Zea mays clone 297738 | 619 | 619 | 38% | 6.00E−174 | 99% |
| | unknown mRNA | | | | | |
| AC084296.12 | Oryza sativa | 51.8 | 51.8 | 7% | 0.006 | 78% |
| | chromosome 3 BAC | | | | | |
| | OSJNBb0024J04 | | | | | |
| | genomic sequence, | | | | | |
| | complete sequence | | | | | |
| AP008209.1 | Oryza sativa (japonica | 51.8 | 51.8 | 7% | 0.006 | 78% |
| | cultivar-group) genomic | | | | | |
| | DNA, chromosome 3 | | | | | |
| BX284754.1 | Neurospora crassa DNA | 50 | 50 | 3% | 0.02 | 93% |
| | linkage group II BAC | | | | | |
| | contig B23G1 | | | | | |
| AC143357.1 | Pan troglodytes BAC | 48.2 | 48.2 | 3% | 0.069 | 96% |
| | clone RP43-171L24 from | | | | | |
| | chromosome 7, complete | | | | | |
| | sequence | | | | | |
| AC003013.1 | Human PAC clone RP1- | 48.2 | 48.2 | 3% | 0.069 | 96% |
| | 205E24 from Xq23, | | | | | |
| | complete sequence | | | | | |
| AL136101.7 | Human DNA sequence | 48.2 | 48.2 | 3% | 0.069 | 96% |
| | from clone RP5-954O23 | | | | | |
| | on chromosome Xq22.2-23, | | | | | |
| | complete sequence | | | | | |
| AM910995.1 | Plasmodium knowlesi | 46.4 | 46.4 | 4% | 0.24 | 82% |
| | strain H chromosome 13, | | | | | |
| | complete genome | | | | | |
| AY573057.1 | Plasmodium knowlesi | 46.4 | 46.4 | 3% | 0.24 | 90% |
| | merozoite surface protein | | | | | |
| | 4 (MSP4) gene, | | | | | |
| | complete cds | | | | | |
| AC120393.16 | Mus musculus | 46.4 | 46.4 | 3% | 0.24 | 89% |
| | chromosome 7, clone | | | | | |
| | RP24-312B12, complete | | | | | |
| | sequence | | | | | |
| AL357510.17 | Human DNA sequence | 46.4 | 46.4 | 4% | 0.24 | 85% |
| | from clone RP11-195F21 | | | | | |
| | on chromosome 10 | | | | | |
| | Contains the 5′ end of a | | | | | |
| | novel gene, complete | | | | | |
| | sequence | | | | | |

### 3). KG_fragment 45

KG_fragment 45/Maize EST 57894155.f01 encodes a protein that is homologous to a hypothetical protein Os06g0473800 of rice (GenBank Accession: NP_{—}001057629). The top 10 homologous sequences identified in the BlastX query are presented in Table 8.

TABLE 8
BLASTX search results of KG_{—}
fragment 45/Hyseq EST 57894155.f01

| | | | | % Iden- |
|---|---|---|---|---|
| Accession | Description | Score | E-value | tities |
| NP_001057629 | Os06g0473800 [Oryza | 176 | 1e−42 | 60 |
| | sativa (japonica | | | |
| | cultivar-group)] | | | |
| EAZ00929 | hypothetical protein | 172 | 2e−41 | 66 |
| | OsI_022161 [Oryza | | | |
| | sativa (indica | | | |
| | cultivar-group)] | | | |
| AAG01171 | seed oleosin isoform 1 | 97 | 1e−18 | 40 |
| | [Fagopyrum esculentum] | | | |
| AAG09751 | oleosin | 91 | 6e−17 | 36 |
| | [Perilla frutescens] | | | |
| AAG24455 | 19 kDa oleosin | 91 | 8e−17 | 36 |
| | [Perilla frutescens] | | | |
| AAB58402 | 15.5 kDa oleosin | 90 | 1e−16 | 45 |
| | [Sesamum indicum] | | | |
| AAB24078 | lipid body membrane | 89 | 2e−16 | 42 |
| | protein [Daucus carota] | | | |
| CAA57994 | high molecular weight | 89 | 2e−16 | 48 |
| | oleosin [Hordeum | | | |
| | vulgare subsp. Vulgare] | | | |
| AAG23840 | oleosin | 89 | 3e−16 | 37 |
| | [Sesamum indicum] | | | |
| ABW90149 | oleosin 2 | 88 | 5e−16 | 35 |
| | [Jatropha curcas] | | | |

The CDS sequence of the gene corresponding to KG_Fragment 45 is shown in SEQ ID NO: 29 and the translated amino acid sequence is shown in SEQ ID NO: 47.

### Identification of the Promoter Region of KG45

For our promoter identification purposes, the sequence upstream of the start codon of the predicted KG_Fragment 45 gene was defined as the promoter p-KG45. To identify this predicted promoter region, the sequence of 57894155.f_o1 was mapped to the BASF Plant Science proprietary genomic DNA sequence database, PUBtigr_maize_genomic_partial_{—}5.0.nt. The reverse complement sequence of a maize genomic DNA sequence, AZM5_{—}29112 (2548 bp) was identified (SEQ ID NO: 83). This 2548 bp sequence harbored the predicted CDS of the corresponding gene to KG_Fragment 45 and about 1.2 kb upstream sequence of the ATG start codon of this gene.

### Isolation of the Promoter Region of KG45 by PCR Amplification

The putative promoter region was isolated via genomic PCR using the following sequence specific primers:

Forward primer:

(SEQ ID NO: 158)

CCAGCCATCGTGCTTGAGTG
Reverse primer:

(SEQ ID NO: 159)

GACGTGGTGGCGATCGCAAG

The expected 1131 bp fragment was amplified from maize genomic DNA, and named as promoter KG45 (p-KG45). Sequence of p-KG45 is shown in SEQ ID NO:11.
BLASTN Results of p_KG45

The top 15 homologous sequences identified in the BlastN query are presented in Table 9.

TABLE 9
BlastN results of p_KG45

| | | Max | Total | Query | | |
|---|---|---|---|---|---|---|
| Accession | Description | score | score | coverage | E value | Max ident |
| EU976834.1 | Zea mays clone | 59 | 59 | 2% | 5.00E−05 | 100% |
| | 991429 unknown | | | | | |
| | mRNA | | | | | |
| CU634021.8 | Zebrafish DNA | 53.6 | 53.6 | 4% | 0.002 | 85% |
| | sequence from | | | | | |
| | clone CH73-96B22 | | | | | |
| | in linkage group 20, | | | | | |
| | complete sequence | | | | | |
| AC158582.2 | Mus musculus | 51.8 | 51.8 | 4% | 0.007 | 83% |
| | chromosome 7, | | | | | |
| | clone RP24- | | | | | |
| | 173K12, complete | | | | | |
| | sequence | | | | | |
| AC102506.9 | Mus musculus | 51.8 | 51.8 | 4% | 0.007 | 80% |
| | chromosome 1, | | | | | |
| | clone RP24- | | | | | |
| | 139E15, complete | | | | | |
| | sequence | | | | | |
| AC114988.21 | Mus musculus | 51.8 | 51.8 | 4% | 0.007 | 83% |
| | chromosome 7, | | | | | |
| | clone RP23-207N5, | | | | | |
| | complete sequence | | | | | |
| AY105760.2 | Zea mays | 50 | 50 | 2% | 0.025 | 100% |
| | PCO070107 mRNA | | | | | |
| | sequence | | | | | |
| DQ485452.1 | Homo sapiens | 50 | 50 | 3% | 0.025 | 89% |
| | protein kinase D1 | | | | | |
| | (PRKD1) gene, | | | | | |
| | complete cds | | | | | |
| AC102004.7 | Mus musculus | 50 | 50 | 4% | 0.025 | 82% |
| | chromosome 15, | | | | | |
| | clone RP24-489M6, | | | | | |
| | complete sequence | | | | | |
| AC158556.9 | Mus musculus | 50 | 50 | 4% | 0.025 | 82% |
| | chromosome 15, | | | | | |
| | clone RP23- | | | | | |
| | 140F20, complete | | | | | |
| | sequence | | | | | |
| AC111275.4 | Rattus norvegicus 4 | 50 | 50 | 5% | 0.025 | 78% |
| | BAC CH230-49L22 | | | | | |
| | (Children's Hospital | | | | | |
| | Oakland Research | | | | | |
| | Institute) complete | | | | | |
| | sequence | | | | | |
| AC097745.8 | Rattus norvegicus 3 | 50 | 50 | 4% | 0.025 | 80% |
| | BAC CH230-11N5 | | | | | |
| | (Children's Hospital | | | | | |
| | Oakland Research | | | | | |
| | Institute) complete | | | | | |
| | sequence | | | | | |
| AL356756.4 | Human | 50 | 50 | 3% | 0.025 | 89% |
| | chromosome 14 | | | | | |
| | DNA sequence | | | | | |
| | BAC C-2503I6 of | | | | | |
| | library CalTech-D | | | | | |
| | from chromosome | | | | | |
| | 14 of Homo sapiens | | | | | |
| | (Human), complete | | | | | |
| | sequence | | | | | |
| AL445884.4 | Human | 50 | 50 | 3% | 0.025 | 89% |
| | chromosome 14 | | | | | |
| | DNA sequence | | | | | |
| | BAC R-419C10 of | | | | | |
| | library RPCI-11 | | | | | |
| | from chromosome | | | | | |
| | 14 of Homo sapiens | | | | | |
| | (Human), complete | | | | | |
| | sequence | | | | | |
| AC199142.9 | Canis familiaris, | 48.2 | 48.2 | 4% | 0.087 | 82% |
| | clone XX-240A15, | | | | | |
| | complete sequence | | | | | |
| AC182436.1 | Mus musculus | 48.2 | 48.2 | 4% | 0.087 | 81% |
| | chromosome 5, | | | | | |
| | clone wi1-1982K15, | | | | | |
| | complete sequence | | | | | |

### 4). KG_fragment 46

KG_fragment 46/Maize EST 62096689.f01 encodes a protein that is homologous to a Cupin family protein of rice (GenBank Accession: ABF95817.1). The top 10 homologous sequences identified in the BlastX query are presented in Table 10.

TABLE 10
BLASTX search results of KG_{—}
fragment 26/Hyseq EST 62096689.f01

| | | | | % Iden- |
|---|---|---|---|---|
| Accession | Description | Score | E-value | tities |
| ABF95817.1 | Cupin family protein, | 313 | 1.00E−98 | 76 |
| | expressed [Oryza | | | |
| | sativa (japonica | | | |
| | cultivar-group)] | | | |
| ABK80758.1 | 7S globulin precursor | 294 | 4.00E−90 | 67 |
| | [Ficus pumila var. | | | |
| | awkeotsang] | | | |
| NP_001050038.1 | Os03g0336100 [Oryza | 313 | 1.00E−98 | 76 |
| | sativa (japonica | | | |
| | cultivar-group)] | | | |
| CAO43605.1 | unnamed protein | 280 | 5.00E−87 | 63 |
| | product [Vitis vinifera] | | | |
| BAA06186.1 | preproMP27-MP32 | 278 | 6.00E−87 | 61 |
| | [Cucurbita | | | |
| | cv. Kurokawa Amakuri] | | | |
| AAT40548.1 | Putative vicilin, identical | 291 | 5.00E−84 | 61 |
| | [Solanum demissum] | | | |
| CAN60323.1 | hypothetical protein | 263 | 7.00E−82 | 63 |
| | [Vitis vinifera] | | | |
| AAC15238.1 | globulin-like protein | 250 | 4.00E−76 | 57 |
| | [Daucus carota] | | | |
| NP_180416.1 | cupin family protein | 253 | 5.00E−76 | 61 |
| | [Arabidopsis thaliana] | | | |
| ABD33075.1 | Cupin region | 249 | 2.00E−72 | 55 |
| | [Medicago truncatula] | | | |

The CDS sequence of the gene corresponding to KG_Fragment 46 is shown in SEQ ID NO 30 and the translated amino acid sequence is shown in SEQ ID NO 48.

### Identification of the Promoter Region of KG46

For our promoter identification purposes, the sequence upstream of the start codon of the predicted KG_Fragment 46 gene was defined as the promoter p-KG46. To identify this predicted promoter region, the sequence of 62096689.f_o1 was mapped to the BASF Plant Science proprietary genomic DNA sequence database, PUB_tigr_maize_genomic_partial_{—}5.0.nt. One maize genomic DNA sequence, AZM5_{—}23539 (2908 bp) was identified (SEQ ID NO: 84). This 2908 bp sequence harbored the predicted CDS of the corresponding gene to KG_Fragment 24 and about 600 bp upstream sequence of the ATG start codon of this gene (SEQ ID NO: 84).

### Isolation of the Promoter Region of KG46 by PCR Amplification

The putative promoter region was isolated via genomic PCR using the following sequence specific primers:

Forward primer:

(SEQ ID NO: 160)

CATTGTTATACATCGGTGATG
Reverse primer:

(SEQ ID NO: 161)

CCTAGCTGGCTTCTTCCAAGC

The expected 563 bp fragment was amplified from maize genomic DNA, and named as promoter KG46 (p-KG46). Sequence of p-KG46 is shown in SEQ ID NO:12.
BLASTN Results of p_KG46

The top 10 homologous sequences identified in the BlastN query are presented in Table 11.

TABLE 11
BlastN results of p_KG46

| | | | | Query | | |
|---|---|---|---|---|---|---|
| | | Max | Total | cover- | | Max |
| Accession | Description | score | score | age | E value | ident |
| EU953111.1 | Zea mays | 156 | 156 | 15% | 1.00E−34 | 100% |
| | clone 1383292 | | | | | |
| | unknown | | | | | |
| | mRNA | | | | | |
| AY105246.1 | Zea mays | 138 | 138 | 13% | 3.00E−29 | 100% |
| | PCO130570 | | | | | |
| | mRNA | | | | | |
| | sequence | | | | | |
| EU971630.1 | Zea mays | 122 | 122 | 37% | 2.00E−24 | 75% |
| | clone 368362 | | | | | |
| | unknown | | | | | |
| | mRNA | | | | | |
| AY455286.1 | Zea mays | 107 | 107 | 22% | 5.00E−20 | 81% |
| | chloroplast | | | | | |
| | phytoene | | | | | |
| | synthase (Y1) | | | | | |
| | gene, | | | | | |
| | complete cds; | | | | | |
| | nuclear gene | | | | | |
| | for chloroplast | | | | | |
| | product | | | | | |
| EU968175.1 | Zea mays | 64.4 | 64.4 | 11% | 5.00E−07 | 85% |
| | clone 316213 | | | | | |
| | unknown | | | | | |
| | mRNA | | | | | |
| AY664417.1 | Zea mays | 46.4 | 46.4 | 24% | 0.15 | 71% |
| | cultivar Mo17 | | | | | |
| | locus 9002, | | | | | |
| | complete | | | | | |
| | sequence | | | | | |
| AP008213.1 | Oryza sativa | 44.6 | 44.6 | 8% | 0.51 | 81% |
| | (japonica | | | | | |
| | cultivar-group) | | | | | |
| | genomic DNA, | | | | | |
| | chromosome 7 | | | | | |
| EU970588.1 | Zea mays | 42.8 | 42.8 | 6% | 1.8 | 89% |
| | clone 347636 | | | | | |
| | unknown | | | | | |
| | mRNA | | | | | |
| EU958640.1 | Zea mays | 42.8 | 42.8 | 5% | 1.8 | 90% |
| | clone 1706905 | | | | | |
| | unknown | | | | | |
| | mRNA | | | | | |
| CP000964.1 | Klebsiella | 42.8 | 42.8 | 6% | 1.8 | 88% |
| | pneumoniae | | | | | |
| | 342, complete | | | | | |
| | genome | | | | | |

### 5). KG_fragment 49

KG_fragment 49/Maize EST 62158447.f01 encodes a protein that is homologous to a hypothetical protein Osl_{—}010295 of rice (GenBank Accession: EAY89062). The top 10 homologous sequences identified in the BlastX query are presented in Table 12.

TABLE 12
BLASTX search results of KG_{—}
fragment 49/Hyseq EST 62158447.f01

| | | | | % Iden- |
|---|---|---|---|---|
| Accession | Description | Score | E-value | tities |
| EAY89062 | hypothetical protein | 1021 | 0.0 | 87 |
| | OsI_010295 [Oryza | | | |
| | sativa (indica | | | |
| | cultivar-group)] | | | |
| ABF94669 | dnaK protein, | 1020 | 0.0 | 87 |
| | expressed [Oryza | | | |
| | sativa (japonica | | | |
| | cultivar-group)] | | | |
| CAN68225 | hypothetical protein | 776 | 0.0 | 65 |
| | [Vitis vinifera] | | | |
| CAO71160 | unnamed protein | 776 | 0.0 | 66 |
| | product [Vitis vinifera] | | | |
| NP_180771 | HSP70T-2; ATP binding | 754 | 0.0 | 64 |
| | [Arabidopsis thaliana] | | | |
| AAM67201 | 70 kD heat shock protein | 749 | 0.0 | 64 |
| | [Arabidopsis thaliana] | | | |
| ACC93947 | heat-shock protein | 297 | 3.00E−78 | 35 |
| | 70 [Hevea brasiliensis] | | | |
| XP_001785822 | predicted protein | 289 | 7.00E−76 | 35 |
| | [Physcomitrella patens | | | |
| | subsp. patens] | | | |
| XP_001783048 | predicted protein | 288 | 1.00E−75 | 35 |
| | [Physcomitrella patens | | | |
| | subsp. patens] | | | |
| XP_001781229 | predicted protein | 288 | 2.00E−75 | 35 |
| | [Physcomitrella patens | | | |
| | subsp. patens] | | | |

The CDS sequence of the gene corresponding to KG_Fragment 49 is shown in SEQ ID NO: 26 and the translated amino acid sequence is shown in SEQ ID NO: 44.

### Identification of the Promoter Region of KG49

For our promoter identification purposes, the sequence upstream of the start codon of the predicted KG_Fragment 49 gene was defined as the promoter p-KG49. To identify this predicted promoter region, the sequence of 62001211.f_o1 was mapped to the BASF Plant Science proprietary genomic DNA sequence database, PUB_tigr_maize_genomic_partial_{—}5.0.nt. The reverse complement sequence of a maize genomic DNA sequence, AZM5_{—}34102 (1719 bp) was identified (SEQ ID NO: 80). This 1719 bp sequence harbored partial predicted CDS of the corresponding gene to KG_Fragment 49 and about 1.2 kb upstream sequence of the ATG start codon of this gene (SEQ ID NO: 80).

### Isolation of the Promoter Region of KG49 by PCR Amplification

The putative promoter region was isolated via genomic PCR using the following sequence specific primers:

Forward primer:

(SEQ ID NO: 162)

GAGCGACCTCGGACTCAGCGGCT
Reverse primer:

(SEQ ID NO: 163)

CCTACAAACAATATTGCATCAG

The expected 1188 bp fragment was amplified from maize genomic DNA, and named as promoter KG49 (p-KG49). Sequence of p-KG49 is shown in SEQ ID NO:8.
BLASTN Results of p_KG49

The 2 plant homologous sequences identified in the BlastN query are presented in Table 13.

TABLE 13
BlastN results of p_KG49

| | | Max | Total | Query | | Max |
|---|---|---|---|---|---|---|
| Accession | Description | score | score | coverage | E value | ident |
| EU957595.1 | Zea mays | 237 | 237 | 12% | 9.00E−59 | 95% |
| | clone | | | | | |
| | 1598693 | | | | | |
| | unknown | | | | | |
| | mRNA | | | | | |
| EU966687.1 | Zea mays | 93.3 | 93.3 | 14% | 2.00E−15 | 72% |
| | clone | | | | | |
| | 296333 | | | | | |
| | unknown | | | | | |
| | mRNA | | | | | |

### 6). KG_fragment 56

KG_fragment 56 has no hits to the BPS in-house Hyseq EST database, but has 100% identities to a sequence disclosed in the patent application, pat_US20040034888A1_{—}3514.

KG_Fragment56/pat_US20040034888A1_{—}3514 encodes a protein that is homologous to a hypothetical protein Os02g0158900 of rice (GenBank Accession: NP_{—}001045960.1). The top 10 homologous sequences identified in the BlastX query are presented in Table 14.

TABLE 14
BLASTX search results of KG_fragment 56

| | | | | % Iden- |
|---|---|---|---|---|
| Accession | Description | Score | E-value | tities |
| NP_001045960.1 | Os02g0158900 [Oryza | 237 | e−128 | 76 |
| | sativa (japonica | | | |
| | cultivar-group)] | | | |
| EAZ21814.1 | hypothetical protein | 233 | e−125 | 76 |
| | OsJ_005297 [Oryza | | | |
| | sativa (japonica | | | |
| | cultivar-group)] | | | |
| CAO62717.1 | unnamed protein | 205 | 2.00E−93 | 60 |
| | product [Vitis vinifera] | | | |
| CAN64662.1 | hypothetical protein | 202 | 3.00E−92 | 59 |
| | [Vitis vinifera] | | | |
| AAF66638.1 | AF143742_1 SNF4 | 167 | 6.00E−74 | 64 |
| | [Lycopersicon | | | |
| | esculentum] | | | |
| AAA91175.1 | Pv42p | 103 | 2.00E−72 | 62 |
| AAO61675.1 | SNF4b [Medicago | 111 | 3.00E−72 | 67 |
| | truncatula] | | | |
| NP_172985.1 | CBS domain- | 108 | 3.00E−69 | 65 |
| | containing protein | | | |
| | [Arabidopsis thaliana] | | | |
| XP_001761144.1 | predicted protein | 163 | 1.00E−66 | 50 |
| | [Physcomitrella | | | |
| | patens subsp. patens] | | | |
| BAC42835.1 | unknown protein | 191 | 7.00E−64 | 46 |
| | [Arabidopsis thaliana] | | | |

The CDS sequence of the gene corresponding to KG_Fragment 56 is shown in SEQ ID NO:21 and the translated amino acid sequence is shown in SEQ ID NO:39.

### Identification of the Promoter Region of KG56

For our promoter identification purposes, the sequence upstream of the start codon of the predicted KG_Fragment 56 gene was defined as the promoter p-KG56. To identify this predicted promoter region, the sequence of 62001211.f_o1 was mapped to the BASF Plant Science proprietary genomic DNA sequence database, PUB_zmdb_genomesurveyseqs.nt, One maize genomic DNA sequence, ZmGSStuc11-12-04.2541.1 (8495 bp) was identified (SEQ ID NO: 75). The first 4.2 kb of ZmGSStuc11-12-04.2541.1 sequence harbored the predicted CDS of the corresponding gene to KG_Fragment 56 and more than 2 kb upstream sequence of the ATG start codon of this gene (SEQ ID NO: 75).

### Isolation of the Promoter Region of KG56 by PCR Amplification

The putative promoter region was isolated via genomic PCR using the following sequence specific primers:

Forward primer:

(SEQ ID NO: 164)

GATTCAGAACATCTGGTCAG
Reverse primer:

(SEQ ID NO: 165)

AGGTTTAGCGAACAAGGC

The expected 1945 bp fragment was amplified from maize genomic DNA, and named as promoter KG56 (p-KG56). Sequence of p-KG56 is shown in SEQ ID NO:3.
BLASTN Results of p_KG56

The top 15 homologous sequences identified in the BlastN query are presented in Table 15.

TABLE 15
BlastN results of p_KG56

| | | Max | Total | Query | | Max |
|---|---|---|---|---|---|---|
| Accession | Description | score | score | coverage | E value | ident |
| EU971086.1 | Zea mays clone | 1088 | 1895 | 54% | 0 | 99% |
| | 357153 unknown | | | | | |
| | mRNA | | | | | |
| AP008208.1 | Oryza sativa | 437 | 956 | 51% | 8.00E−119 | 87% |
| | (japonica cultivar- | | | | | |
| | group) genomic DNA, | | | | | |
| | chromosome 2 | | | | | |
| AP004843.2 | Oryza sativa | 437 | 956 | 51% | 8.00E−119 | 87% |
| | Japonica Group | | | | | |
| | genomic DNA, | | | | | |
| | chromosome 2, BAC | | | | | |
| | clone: B1103G11 | | | | | |
| NM_001052494.1 | Oryza sativa | 430 | 975 | 41% | 1.00E−116 | 87% |
| | (japonica cultivar- | | | | | |
| | group) | | | | | |
| | Os02g0158800 | | | | | |
| | (Os02g0158800) | | | | | |
| | mRNA, complete cds | | | | | |
| AK119177.1 | Oryza sativa | 430 | 975 | 41% | 1.00E−116 | 87% |
| | Japonica Group | | | | | |
| | cDNA clone: 001-037- | | | | | |
| | G06, full insert | | | | | |
| | sequence | | | | | |
| AK065389.1 | Oryza sativa | 430 | 975 | 41% | 1.00E−116 | 87% |
| | Japonica Group | | | | | |
| | cDNA | | | | | |
| | clone: J013021B10, | | | | | |
| | full insert sequence | | | | | |
| BT041386.1 | Zea mays full-length | 242 | 670 | 41% | 4.00E−60 | 83% |
| | cDNA clone | | | | | |
| | ZM_BFc0117N09 | | | | | |
| | mRNA, complete cds | | | | | |
| EU976055.1 | Zea mays clone | 239 | 659 | 41% | 4.00E−59 | 82% |
| | 509800 unknown | | | | | |
| | mRNA | | | | | |
| AP008212.1 | Oryza sativa | 239 | 731 | 43% | 4.00E−59 | 84% |
| | (japonica cultivar- | | | | | |
| | group) genomic DNA, | | | | | |
| | chromosome 6 | | | | | |
| AP005395.3 | Oryza sativa | 239 | 683 | 43% | 4.00E−59 | 84% |
| | Japonica Group | | | | | |
| | genomic DNA, | | | | | |
| | chromosome 6, PAC | | | | | |
| | clone: P0623A10 | | | | | |
| NM_001064941.1 | Oryza sativa | 237 | 663 | 41% | 2.00E−58 | 83% |
| | (japonica cultivar- | | | | | |
| | group) | | | | | |
| | Os06g0687400 | | | | | |
| | (Os06g0687400) | | | | | |
| | mRNA, partial cds | | | | | |
| AK072400.1 | Oryza sativa | 237 | 663 | 41% | 2.00E−58 | 83% |
| | Japonica Group | | | | | |
| | cDNA | | | | | |
| | clone: J023078C17, | | | | | |
| | full insert sequence | | | | | |
| AK060934.1 | Oryza sativa | 237 | 656 | 41% | 2.00E−58 | 83% |
| | Japonica Group | | | | | |
| | cDNA clone: 006-201- | | | | | |
| | B09, full insert | | | | | |
| | sequence | | | | | |
| AP001298.1 | Arabidopsis thaliana | 221 | 406 | 46% | 1.00E−53 | 77% |
| | genomic DNA, | | | | | |
| | chromosome 3, BAC | | | | | |
| | clone: F20C19 | | | | | |
| BT009221.1 | Triticum aestivum | 210 | 614 | 41% | 2.00E−50 | 82% |
| | clone | | | | | |
| | wle1n.pk0074.b4: fis, | | | | | |
| | full insert mRNA | | | | | |
| | sequence | | | | | |

### 7). KG_fragment 103

KG_fragment 103/Maize EST ZM07MC01323_{—}57619299 encodes a Maize Cytochrome P450 78A1 protein (GenBank Accession: NP_{—}001106069.1). The top 10 homologous sequences identified in the BlastX query are presented in Table 16.

TABLE 16
BLASTXsearch results of KG_{—}
fragment_103/EST ZM07MC01323_57619299

| | | | | % Iden- |
|---|---|---|---|---|
| Accession | Description | Score | E-value | tities |
| NP_001106069.1 | Cytochrome P450 | 1057 | 0.0 | 100 |
| | 78A1 [Zea mays] | | | |
| CAO70823.1 | unnamed protein | 370 | 0.0 | 72 |
| | product [Vitis vinifera] | | | |
| CAN73323.1 | hypothetical protein | 367 | 0.0 | 71 |
| | [Vitis vinifera] | | | |
| EAY78409.1 | hypothetical protein | 635 | 0.0 | 84 |
| | OsI_032368 [Oryza | | | |
| | sativa (indica | | | |
| | cultivar-group)] | | | |
| NP_001064552.1 | Os10g0403000 [Oryza | 634 | 0.0 | 84 |
| | sativa (japonica | | | |
| | cultivar-group)] | | | |
| BAC76730.1 | cytochrome P450 | 632 | 0.0 | 83 |
| | 78A11 [Oryza sativa | | | |
| | Japonica Group] | | | |
| EAY79271.1 | hypothetical protein | 634 | e−179 | 84 |
| | OsI_033230 [Oryza | | | |
| | sativa (indica | | | |
| | cultivar-group)] | | | |
| O65012 | C78A4_PINRA | 354 | e−171 | 65 |
| | Cytochrome P450 | | | |
| | 78A4 | | | |
| CAO71766.1 | unnamed protein | 218 | e−163 | 70 |
| | product [Vitis vinifera] | | | |
| XP_001771134.1 | predicted protein | 180 | e−158 | 54 |
| | [Physcomitrella | | | |
| | patens subsp. patens] | | | |

The CDS sequence of the gene corresponding to KG_Fragment 103 is shown SEQ ID NO: 31 and the translated amino acid sequence is shown in SEQ ID NO: 49.

### Identification of the Promoter Region of KG103

For our promoter identification purposes, the sequence upstream of the start codon of the predicted KG_Fragment 103 gene was defined as the promoter p-KG103. To identify this predicted promoter region, the sequence of EST ZM07MC01323_{—}57619299 was mapped to the BASF Plant Science proprietary genomic DNA sequence database, PUB_zmdb_genomesurveyseqs.nt. The reverse complement sequence of a maize genomic DNA sequence, ZmGSStuc11-12-04.9475.1 (5105 bp) was identified (SEQ ID NO: 85). This 5105 bp sequence harbored the predicted CDS of the corresponding gene to KG_Fragment 103 and about 1.2 kb upstream sequence of the ATG start codon of this gene.

### Isolation of the Promoter Region of KG103 by PCR Amplification

The putative promoter region was isolated via genomic PCR using the following sequence specific primers:

Forward primer:

(SEQ ID NO: 166)

ATCATCACCCTACCCCGAGCT
Reverse primer:

(SEQ ID NO: 167)

GACGAGTTGTTCTGGCTAG

The expected 991 bp fragment was amplified from maize genomic DNA, and named as promoter KG103 (p-KG103). Sequence of p-KG103 is shown in SEQ ID NO:13.
BLASTN Results of p_KG103

The top 25 homologous sequences identified in the BlastN query are presented in Table 17.

TABLE 17
BlastN results of p_KG103

| | | Max | Total | Query | | Max |
|---|---|---|---|---|---|---|
| Accession | Description | score | score | coverage | E value | ident |
| AC157319.2 | Zea mays clone | 1079 | 3747 | 66% | 0 | 96% |
| | ZMMBBb-136E2, | | | | | |
| | complete sequence | | | | | |
| AY530952.1 | Zea mays unknown | 1068 | 2126 | 66% | 0 | 96% |
| | (Z576C20.2), putative | | | | | |
| | heme oxygenase 1 | | | | | |
| | (Z576C20.3), | | | | | |
| | anthocyanin | | | | | |
| | biosynthesis regulatory | | | | | |
| | protein PI1_B73 | | | | | |
| | (Z576C20.4), putative | | | | | |
| | growth-regulating | | | | | |
| | factor 1 (Z576C20.6), | | | | | |
| | and putative | | | | | |
| | aminoalcoholphospho- | | | | | |
| | transferase | | | | | |
| | (Z576C20.14) genes, | | | | | |
| | complete cds; and | | | | | |
| | putative receptor | | | | | |
| | protein kinase | | | | | |
| | (Z576C20.21) gene, | | | | | |
| | partial cds | | | | | |
| EU952200.1 | Zea mays clone | 1058 | 1058 | 66% | 0 | 95% |
| | 1221105 unknown | | | | | |
| | mRNA | | | | | |
| EU338354.1 | Zea mays cultivar W22 | 1050 | 5123 | 68% | 0 | 95% |
| | bz gene locus, | | | | | |
| | complete sequence | | | | | |
| AF391808.3 | Zea mays cultivar McC | 1050 | 5117 | 68% | 0 | 95% |
| | bz locus region | | | | | |
| AC165176.2 | Zea mays clone | 1031 | 1.20E+04 | 66% | 0 | 94% |
| | ZMMBBb-177G21, | | | | | |
| | complete sequence | | | | | |
| AY883559.2 | Zea mays cultivar | 1018 | 3526 | 66% | 0 | 94% |
| | inbred line B73 | | | | | |
| | teosinte glume | | | | | |
| | architecture 1 (tga1) | | | | | |
| | gene, complete cds | | | | | |
| AF466646.1 | Zea mays putative | 1007 | 1007 | 66% | 0 | 94% |
| | transposase | | | | | |
| | (Z195D10.1) gene, | | | | | |
| | partial cds; glycyl-tRNA | | | | | |
| | synthetase | | | | | |
| | (Z195D10.2), ornithine | | | | | |
| | carbamoyltransferase | | | | | |
| | (Z195D10.3), putative | | | | | |
| | gag protein | | | | | |
| | (Z195D10.5), putative | | | | | |
| | SET-domain | | | | | |
| | transcriptional | | | | | |
| | regulator (Z195D10.7), | | | | | |
| | putative oxysterol- | | | | | |
| | binding protein | | | | | |
| | (Z195D10.8), putative | | | | | |
| | polyprotein | | | | | |
| | (Z195D10.9), putative | | | | | |
| | oxysterol-binding | | | | | |
| | protein (Z195D10.10), | | | | | |
| | putative gag-pol | | | | | |
| | polyprotein | | | | | |
| | (Z195D10.11), putative | | | | | |
| | phosphatidylinositol-4- | | | | | |
| | phosphate-5-kinase | | | | | |
| | (Z195D10.12), | | | | | |
| | hypothetical protein | | | | | |
| | (Z195D10.15), putative | | | | | |
| | gag-pol polyprotein | | | | | |
| | (Z195D10.16), putative | | | | | |
| | polyprotein | | | | | |
| | (Z195D10.17), putative | | | | | |
| | retrotransposon protein | | | | | |
| | (Z195D10.18), and | | | | | |
| | prpol (Z195D10.19) | | | | | |
| | genes, complete cds; | | | | | |
| | and putative teosinte | | | | | |
| | branched2 | | | | | |
| | (Z195D10.20) gene, | | | | | |
| | partial cds | | | | | |
| AC152495.1 | Zea mays BAC clone | 1003 | 1984 | 66% | 0 | 94% |
| | Z486N13, complete | | | | | |
| | sequence | | | | | |
| AF123535.1 | Zea mays alcohol | 991 | 1964 | 67% | 0 | 93% |
| | dehydrogenase 1 | | | | | |
| | (adh1) gene, adh1-F | | | | | |
| | allele, complete cds | | | | | |
| AY691949.1 | Zea mays alcohol | 991 | 1970 | 67% | 0 | 93% |
| | dehydrogenase 1 | | | | | |
| | (adh1A) gene, | | | | | |
| | complete cds; Fourf | | | | | |
| | copia_LTR and Huck | | | | | |
| | gypsy_LTR | | | | | |
| | retrotransposons, | | | | | |
| | complete sequence; | | | | | |
| | Opie2 copia_LTR | | | | | |
| | retrotransposon Zeon | | | | | |
| | gypsy_LTR and Opie1 | | | | | |
| | copia_LTR | | | | | |
| | retrotransposons, | | | | | |
| | complete sequence; Ji | | | | | |
| | copia_LTR | | | | | |
| | retrotransposon, | | | | | |
| | complete sequence; | | | | | |
| | and unknown protein | | | | | |
| | (adh1B), cyclin H-1 | | | | | |
| | (adh1C), unknown | | | | | |
| | protein (adh1D), | | | | | |
| | hypothetical protein | | | | | |
| | (adh1E), and unknown | | | | | |
| | protein (adh1F) genes, | | | | | |
| | complete cds | | | | | |
| DQ417752.1 | Zea mays B73 | 984 | 5530 | 66% | 0 | 93% |
| | pathogenesis-related | | | | | |
| | protein 2 and GASA- | | | | | |
| | like protein genes, | | | | | |
| | complete cds | | | | | |
| AF050440.1 | Zea mays | 982 | 982 | 66% | 0 | 93% |
| | retrotransposon Huck- | | | | | |
| | 2 3′ LTR, partial | | | | | |
| | sequence | | | | | |
| DQ002408.1 | Zea mays gypsy | 976 | 3501 | 66% | 0 | 93% |
| | retrotransposon huck, | | | | | |
| | and copia | | | | | |
| | retrotransposon ji, | | | | | |
| | complete sequence; | | | | | |
| | and helitron | | | | | |
| | Mo17_14594, | | | | | |
| | complete sequence | | | | | |
| U68404.1 | Zea mays | 973 | 973 | 66% | 0 | 93% |
| | retrotransposon Huck- | | | | | |
| | 2 5′ LTR and primer | | | | | |
| | binding site DNA | | | | | |
| | sequence | | | | | |
| AY530950.1 | Zea mays putative zinc | 971 | 4162 | 67% | 0 | 93% |
| | finger protein | | | | | |
| | (Z438D03.1), unknown | | | | | |
| | (Z438D03.5), epsilon- | | | | | |
| | COP (Z438D03.6), | | | | | |
| | putative kinase | | | | | |
| | (Z438D03.7), unknown | | | | | |
| | (Z438D03.25), and C1- | | | | | |
| | B73 (Z438D03.27) | | | | | |
| | genes, complete cds | | | | | |
| AC160211.1 | Genomic seqeunce for | 969 | 4518 | 66% | 0 | 93% |
| | Zea mays BAC clone | | | | | |
| | ZMMBBb0448F23, | | | | | |
| | complete sequence | | | | | |
| AC157487.1 | Genomic sequence for | 966 | 6419 | 66% | 0 | 93% |
| | Zea mays clone | | | | | |
| | ZMMBBb0614J24, | | | | | |
| | from chromosome 8, | | | | | |
| | complete sequence | | | | | |
| AY530951.1 | Zea mays putative | 964 | 4254 | 66% | 0 | 93% |
| | growth-regulating | | | | | |
| | factor 1 (Z214A02.12), | | | | | |
| | putative 40S ribosomal | | | | | |
| | protein S8 | | | | | |
| | (Z214A02.25), and | | | | | |
| | putative casein kinase | | | | | |
| | I (Z214A02.27) genes, | | | | | |
| | complete cds | | | | | |
| AY664416.1 | Zea mays cultivar | 958 | 3019 | 66% | 0 | 92% |
| | Mo17 locus bz, | | | | | |
| | complete sequence | | | | | |
| AY555142.1 | Zea mays BAC clone | 951 | 2719 | 66% | 0 | 92% |
| | c573F08, complete | | | | | |
| | sequence | | | | | |
| AY664419.1 | Zea mays cultivar | 951 | 4061 | 66% | 0 | 92% |
| | Mo17 locus 9009, | | | | | |
| | complete sequence | | | | | |
| AC165174.2 | Zea mays clone | 921 | 1836 | 66% | 0 | 91% |
| | ZMMBBb-127F19, | | | | | |
| | complete sequence | | | | | |
| AC165173.2 | Zea mays clone | 921 | 2326 | 66% | 0 | 91% |
| | ZMMBBb-125O19, | | | | | |
| | complete sequence | | | | | |
| DQ493649.1 | Zea mays cultivar | 915 | 3472 | 66% | 0 | 91% |
| | Coroico bz locus | | | | | |
| | region | | | | | |

### 8). KG_fragment 119

KG_fragment 119/Maize EST ZM07MC15086_{—}59463108 encodes a protein that is homologous to a hypothetical protein Os09g0433900 of rice (GenBank Accession: NP_{—}001063248). The top 10 homologous sequences identified in the BlastX query are presented in Table 18

TABLE 18
BLASTX search results of KG_fragment
119/Hyseq EST ZM07MC15086_59463108

| | | | | % Iden- |
|---|---|---|---|---|
| Accession | Description | Score | E-value | tities |
| NP_001063248 | Os09g0433900 [Oryza | 696 | 0.0 | 67 |
| | sativa (japonica | | | |
| | cultivar-group)] | | | |
| EAZ09214 | hypothetical protein | 657 | 0.0 | 67 |
| | OsI_030446 [Oryza | | | |
| | sativa (indica | | | |
| | cultivar-group)] | | | |
| EAZ44840 | hypothetical protein | 611 | e−173 | 61 |
| | OsJ_028323 [Oryza | | | |
| | sativa (japonica | | | |
| | cultivar-group)] | | | |
| AAV64199 | putative alanine amino- | 571 | e−161 | 56 |
| | transferase [Zea mays] | | | |
| AAV64237 | putative alanine amino- | 570 | e−160 | 56 |
| | transferase [Zea mays]. | | | |
| BAC79995 | putative alanine amino- | 559 | e−157 | 60 |
| | transferase [Oryza sativa | | | |
| | Japonica Group] | | | |
| EAZ40671 | hypothetical protein | 556 | e−156 | 58 |
| | OsJ_024154 [Oryza | | | |
| | sativa (japonica | | | |
| | cultivar-group)] | | | |
| EAZ04721 | hypothetical protein | 555 | e−156 | 58 |
| | OsI_025953 [Oryza | | | |
| | sativa (indica | | | |
| | cultivar-group)] | | | |
| CAO45546 | unnamed protein | 555 | e−156 | 58 |
| | product [Vitis vinifera] | | | |
| CAA49199 | alanine aminotransferase | 553 | e−155 | 57 |
| | [Panicum miliaceum] | | | |

The CDS sequence of the gene corresponding to KG_Fragment 119 is shown in SEQ ID NO: 32 and the translated amino acid sequence is shown in SEQ ID NO:50.

### Identification of the Promoter Region of KG119

For our promoter identification purposes, the sequence upstream of the start codon of the predicted KG_Fragment 119 gene was defined as the promoter p-KG119. To identify this predicted promoter region, the sequence of ZM07MC15086_{—}59463108 was mapped to the BASF Plant Science proprietary genomic DNA sequence database, PUB_tigr_maize_genomic_partial_{—}5.0.nt. One maize genomic DNA sequence, AZM5_{—}10092 (8208 bp SEQ ID NO: 86) was identified. The reverse complement sequence of this sequence harbored the predicted CDS of the corresponding gene to KG_Fragment 119 and more than 2 kb upstream sequence of the ATG start codon of this gene (SEQ ID NO: 86).

### Isolation of the Promoter Region of KG119 by PCR Amplification

The putative promoter region was isolated via genomic PCR using the following sequence specific primers:

Forward primer:

(SEQ ID NO: 168)

CTTCCGATAAAAATATTTGGAAC
Reverse primer:

(SEQ ID NO: 169)

GTACGACATGGCGCGTCGG

The expected 2519 bp fragment was amplified from maize genomic DNA, and anotated as promoter KG119 (p-KG119). Sequence of p-KG119 is shown in SEQ ID NO:14
BLASTN Results of p_KG119

The top 15 homologous sequences identified in the BlastN query are presented in Table 19.

TABLE 19
BlastN results of p_KG119

| | | Max | Total | Query | | Max |
|---|---|---|---|---|---|---|
| Accession | Description | score | score | coverage | E value | ident |
| EU966511.1 | Zea mays clone 294961 | 821 | 1032 | 23% | 0 | 99% |
| | unknown mRNA | | | | | |
| AF215823.2 | Zea mays T cytoplasm | 545 | 545 | 15% | 3.00E−151 | 92% |
| | male sterility restorer factor | | | | | |
| | 2 (rf2a) gene, rf2a-B73 | | | | | |
| | allele, complete cds | | | | | |
| AC157977.1 | Genomic sequence for Zea | 535 | 535 | 15% | 5.00E−148 | 91% |
| | mays chromosome 8 BAC | | | | | |
| | clone ZMMBBb0284N04, | | | | | |
| | complete sequence | | | | | |
| EU957455.1 | Zea mays clone 1592915 | 531 | 531 | 14% | 6.00E−147 | 92% |
| | unknown mRNA | | | | | |
| AY662985.1 | Zea luxurians YZ1 (yz1) | 504 | 716 | 21% | 9.00E−139 | 89% |
| | gene, complete cds; | | | | | |
| | transposons mPIF-like | | | | | |
| | element and frequent flyer, | | | | | |
| | complete sequence; and | | | | | |
| | NADPH-dependent | | | | | |
| | reductase (a1) gene, partial | | | | | |
| | cds | | | | | |
| AC165178.2 | Zea mays clone ZMMBBb- | 497 | 497 | 14% | 1.00E−136 | 90% |
| | 272P17, complete | | | | | |
| | sequence | | | | | |
| EF659468.1 | Zea mays clone BAC | 484 | 621 | 14% | 8.00E−133 | 90% |
| | b0288K09 AP2 domain | | | | | |
| | transcription factor | | | | | |
| | (Rap2.7) gene, partial cds | | | | | |
| AJ005343.1 | Zea mays Ama gene | 464 | 464 | 14% | 8.00E−127 | 89% |
| | encoding single-subunit | | | | | |
| | RNA polymerase | | | | | |
| AC165171.2 | Zea mays clone CH201- | 462 | 462 | 15% | 3.00E−126 | 87% |
| | 145P10, complete | | | | | |
| | sequence | | | | | |
| AF466646.1 | Zea mays putative | 461 | 606 | 14% | 9.00E−126 | 87% |
| | transposase (Z195D10.1) | | | | | |
| | gene, partial cds; glycyl- | | | | | |
| | tRNA synthetase | | | | | |
| | (Z195D10.2), ornithine | | | | | |
| | carbamoyltransferase | | | | | |
| | (Z195D10.3), putative gag | | | | | |
| | protein (Z195D10.5), | | | | | |
| | putative SET-domain | | | | | |
| | transcriptional regulator | | | | | |
| | (Z195D10.7), putative | | | | | |
| | oxysterol-binding protein | | | | | |
| | (Z195D10.8), putative | | | | | |
| | polyprotein (Z195D10.9), | | | | | |
| | putative oxysterol-binding | | | | | |
| | protein (Z195D10.10), | | | | | |
| | putative gag-pol | | | | | |
| | polyprotein (Z195D10.11), | | | | | |
| | putative | | | | | |
| | phosphatidylinositol-4- | | | | | |
| | phosphate-5-kinase | | | | | |
| | (Z195D10.12), hypothetical | | | | | |
| | protein (Z195D10.15), | | | | | |
| | putative gag-pol | | | | | |
| | polyprotein (Z195D10.16), | | | | | |
| | putative polyprotein | | | | | |
| | (Z195D10.17), putative | | | | | |
| | retrotransposon protein | | | | | |
| | (Z195D10.18), and prpol | | | | | |
| | (Z195D10.19) genes, | | | | | |
| | complete cds; and putative | | | | | |
| | teosinte branched2 | | | | | |
| | (Z195D10.20) gene, partial | | | | | |
| | cds | | | | | |
| AY789036.1 | Zea mays subsp. | 461 | 461 | 14% | 9.00E−126 | 88% |
| | parviglumis floricaula/leafy- | | | | | |
| | like 2 (zfl2) gene, complete | | | | | |
| | cds | | | | | |
| AC165174.2 | Zea mays clone ZMMBBb- | 459 | 959 | 19% | 3.00E−125 | 88% |
| | 127F19, complete | | | | | |
| | sequence | | | | | |
| AF448416.1 | Zea mays B73 | 459 | 459 | 14% | 3.00E−125 | 87% |
| | chromosome 9S bz | | | | | |
| | genomic region | | | | | |
| AF416310.1 | Zea mays clone mPIF268 | 459 | 459 | 14% | 3.00E−125 | 88% |
| | mPIF miniature inverted- | | | | | |
| | repeat transposable | | | | | |
| | element | | | | | |
| DQ493647.1 | Zea mays cultivar NalTel | 453 | 453 | 14% | 1.00E−123 | 86% |
| | bz locus region | | | | | |

### 9). KG_fragment 129

KG_fragment 129/maize EST 62092959.f01 encodes a protein that is homologous to a maize unknown protein (GenBank Accession: ACF78165.1). The top 10 homologous sequences identified in the BlastX query are presented in Table 20.

TABLE 20
BLASTX search results of KG_{—}
fragment 129/Hyseq EST 62092959.f01

| | | | | % iden- |
|---|---|---|---|---|
| Accession | Description | Score | E-value | tities |
| ACF78165.1 | unknown[Zea Mays]. | 513 | e−143 | 91 |
| ACF83516.1 | unknown [Zea mays] | 401 | e−110 | 69 |
| ACF86030.1 | unknown [Zea mays] | 243 | 7e−96 | 69 |
| ACF78865.1 | unknown [Zea mays] | 243 | 1e−84 | 69 |
| EAY82651.1 | hypothetical protein | 129 | 3e−42 | 45 |
| | OsI_036610 | | | |
| NP_001066495.1 | Os12g0247700 [Oryza | 121 | 1e−39 | 44 |
| | sativa (japonica | | | |
| | cultivar-group)] | | | |
| NP_001066367.1 | Os12g0198700 [Oryza | 88 | 2e−35 | 47 |
| | sativa (japonica | | | |
| | cultivar-group)] | | | |
| ABE11623.1 | unknown [Oryza | 102 | 6e−34 | 41 |
| | sativa (japonica | | | |
| | cultivar-group)] | | | |
| ABS82785.1 | jasmonate-induced | 92 | 1e−32 | 51 |
| | protein | | | |
| | [Triticum aestivum] | | | |
| AAR20919.1 | jasmonate-induced | 91 | 3e−32 | 50 |
| | protein | | | |
| | [Triticum aestivum] | | | |

The CDS sequence of the gene corresponding to KG_Fragment 129 is shown in SEQ ID NO: 22 and the translated amino acid sequence is shown in SEQ ID NO:40.

### Identification of the Promoter Region of KG129

For our promoter identification purposes, the sequence upstream of the start codon of the predicted KG_Fragment 129 gene was defined as the promoter p-KG129. To identify this predicted promoter region, the sequence of 62092959.f_o1 was mapped to the BASF Plant Science proprietary genomic DNA sequence database, PUB_tigr_maize_genomic_partial_{—}5.0.nt. One maize genomic DNA sequence, AZM5_{—}91706 (2131 bp) was identified (SEQ ID NO: 76). This 2131 bp sequence harbored the predicted CDS of the corresponding gene to KG_Fragment 129 and about 600 bp upstream sequence of the ATG start codon of this gene.

### Isolation of the Promoter Region of KG129by PCR Amplification

The putative promoter region was isolated via genomic PCR using the following sequence specific primers:

Forward primer:

(SEQ ID NO: 170)

GCCAGTGCTAATGATATTTA
Reverse primer:

(SEQ ID NO: 171)

ATGCACCTACTCGGCGGTG

The expected 512 bp fragment was amplified from maize genomic DNA, and annotated as promoter KG129 (p-KG129). Sequence of p-KG129 is shown in SEQ ID NO:4.
BLASTN Results of p_KG129

The top 20 homologous sequences identified in the BlastN query are presented in Table 21.

TABLE 21
BlastN results of p_KG129

| | | Max | Total | Query | | Max |
|---|---|---|---|---|---|---|
| Accession | Description | score | score | coverage | E value | ident |
| EU241905.1 | Zea mays ZCN14 (ZCN14) | 230 | 353 | 38% | 6.00E−57 | 86% |
| | gene, complete cds | | | | | |
| AY682272.1 | Zea mays subsp. mexicana | 228 | 465 | 37% | 2.00E−56 | 86% |
| | barren stalk 1 gene, | | | | | |
| | promoter region I | | | | | |
| AY682271.1 | Zea mays subsp. mexicana | 228 | 465 | 37% | 2.00E−56 | 86% |
| | barren stalk 1 gene, | | | | | |
| | promoter region I | | | | | |
| AY682262.1 | Zea mays barren stalk 1 | 228 | 459 | 37% | 2.00E−56 | 86% |
| | gene, promoter region I | | | | | |
| AY682258.1 | Zea mays barren stalk 1 | 228 | 459 | 37% | 2.00E−56 | 86% |
| | gene, promoter region I | | | | | |
| AY682256.1 | Zea mays barren stalk 1 | 228 | 459 | 37% | 2.00E−56 | 86% |
| | gene, promoter region I | | | | | |
| AY743721.1 | Zea mays subsp. | 228 | 459 | 37% | 2.00E−56 | 86% |
| | parviglumis cultivar INIFAP- | | | | | |
| | JSG 374 barren stalk 1 | | | | | |
| | gene, promoter I region | | | | | |
| AY682254.1 | Zea mays barren stalk 1 | 224 | 455 | 37% | 2.00E−55 | 86% |
| | gene, promoter region I | | | | | |
| AY743723.1 | Zea mays subsp. | 215 | 283 | 37% | 1.00E−52 | 85% |
| | parviglumis cultivar | | | | | |
| | CIMMYT-11355 barren stalk | | | | | |
| | 1 gene, promoter I region | | | | | |
| AY753906.1 | Zea mays subsp. | 206 | 206 | 37% | 6.00E−50 | 84% |
| | parviglumis barren stalk 1 | | | | | |
| | gene, promoter I region | | | | | |
| AY683001.1 | Zea mays cultivar B73 | 201 | 527 | 38% | 3.00E−48 | 85% |
| | barren stalk1 (BA1) gene, | | | | | |
| | complete cds | | | | | |
| AY682281.1 | Zea mays subsp. | 199 | 541 | 37% | 9.00E−48 | 85% |
| | parviglumis barren stalk 1 | | | | | |
| | gene, promoter region I | | | | | |
| AY682274.1 | Zea mays subsp. | 199 | 525 | 37% | 9.00E−48 | 85% |
| | parviglumis barren stalk 1 | | | | | |
| | gene, promoter region I | | | | | |
| AY682273.1 | Zea mays subsp. | 199 | 525 | 37% | 9.00E−48 | 85% |
| | parviglumis barren stalk 1 | | | | | |
| | gene, promoter region I | | | | | |
| AY682270.1 | Zea mays subsp. mexicana | 199 | 525 | 37% | 9.00E−48 | 85% |
| | barren stalk 1 gene, | | | | | |
| | promoter region I | | | | | |
| AY682269.1 | Zea mays subsp. mexicana | 199 | 525 | 37% | 9.00E−48 | 85% |
| | barren stalk 1 gene, | | | | | |
| | promoter region I | | | | | |
| AY682268.1 | Zea mays barren stalk 1 | 199 | 525 | 37% | 9.00E−48 | 85% |
| | gene, promoter region I | | | | | |
| AY682267.1 | Zea mays barren stalk 1 | 199 | 525 | 37% | 9.00E−48 | 85% |
| | gene, promoter region I | | | | | |
| AY682266.1 | Zea mays barren stalk 1 | 199 | 525 | 37% | 9.00E−48 | 85% |
| | gene, promoter region I | | | | | |
| AY682265.1 | Zea mays barren stalk 1 | 199 | 525 | 37% | 9.00E−48 | 85% |
| | gene, promoter region I | | | | | |

### Example 5

### Place Analysis of the Promoters

Cis-acting motifs in the promoter regions were identified using PLACE (a database of Plant Cis-acting Regulatory DNA elements) using the Genomatix database suite.

1.) p-KG24

PLACE analysis results of p-KG24 are listed in Table 22. No TATA box motif is found in this promoter, but there are 2 CAAT Box motifs at nucleotide position 191-195 and 247-251 of the forward strand, respectively. These CAAT Box motifs are distal from the 3′ end of the promoter and therefore may not be functional motifs.

TABLE 22
PLACE analysis results of the 1507bp promoter of p-KG24

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM156 | L1BOXATPDF1 | 2 | 9 | − | 0 | 1 | TAAATGTA |
| FAM290 | GT1GMSCAM4 | 56 | 61 | + | 0 | 1 | GAAAAA |
| FAM311 | EECCRCAH1 | 68 | 74 | − | 0 | 1 | GATTTAC |
| FAM087 | BOXIINTPATPB | 76 | 81 | + | 0 | 1 | ATAGAA |
| FAM303 | OSE1ROOTNODULE | 90 | 96 | − | 0 | 1 | AAAGATG |
| FAM012 | IBOXCORE | 126 | 132 | + | 0 | 1 | GATAACT |
| FAM267 | NTBBF1ARROLB | 130 | 136 | + | 0 | 1 | ACTTTAG |
| FAM267 | TAAAGSTKST1 | 131 | 137 | − | 0 | 1 | TCTAAAG |
| FAM272 | SV40COREENHAN | 143 | 150 | + | 0 | 1 | GTGGAATG |
| FAM322 | BIHD1OS | 149 | 153 | + | 0 | 1 | TGTCA |
| FAM027 | -10PEHVPSBD | 154 | 159 | + | 0 | 1 | TATTCT |
| FAM100 | CCAATBOX1 | 191 | 195 | + | 0 | 1 | CCAAT |
| FAM305 | ANAERO1CONSENSUS | 212 | 218 | + | 0 | 1 | AAACAAA |
| FAM039 | AACACOREOSGLUB1 | 213 | 219 | + | 0 | 1 | AACAAAC |
| FAM325 | MYBCOREATCYCB1 | 217 | 221 | + | 0 | 1 | AACGG |
| FAM302 | SORLIP2AT | 220 | 230 | + | 0 | 1 | GGGGCCTTATT |
| FAM310 | CPBCSPOR | 227 | 232 | + | 0 | 1 | TATTAG |
| FAM311 | EECCRCAH1 | 236 | 242 | − | 0 | 1 | GAATTCC |
| FAM100 | CCAATBOX1 | 247 | 251 | + | 0 | 1 | CCAAT |
| FAM311 | EECCRCAH1 | 272 | 278 | + | 0 | 1 | GATTTCC |
| FAM290 | GT1GMSCAM4 | 289 | 294 | + | 0 | 1 | GAAAAA |
| FAM006 | HDZIP2ATATHB2 | 318 | 326 | + | 0 | 1 | TAATAATTA |
| FAM170 | MYBGAHV | 330 | 336 | − | 0 | 1 | TAACAAA |
| FAM010 | WBOXNTCHN48 | 332 | 346 | − | 0 | 1 | GCTGACCTTTTAACA |
| FAM205 | PYRIMIDINEBOXOSRAM | 336 | 341 | − | 0 | 1 | CCTTTT |
| FAM010 | QELEMENTZMZM13 | 337 | 351 | + | 0 | 1 | AAAGGTCAGCTTCCC |
| FAM202 | -300ELEMENT | 364 | 372 | + | 0 | 1 | TGTAAAAGC |
| FAM302 | SITEIIATCYTC | 365 | 375 | − | 0 | 1 | TGGGCTTTTAC |
| FAM003 | MYBPLANT | 385 | 395 | − | 0 | 1 | AACCAAACAGA |
| FAM171 | MYBPZM | 392 | 398 | − | 0 | 1 | CCCAACC |
| FAM302 | SITEIIATCYTC | 395 | 405 | + | 0 | 1 | TGGGCTGTGGC |
| FAM002 | SORLIP1AT | 399 | 411 | − | 0 | 1 | TTCACAGCCACAG |
| FAM290 | GT1GMSCAM4 | 409 | 414 | + | 0 | 1 | GAAAAA |
| FAM302 | SITEIIATCYTC | 423 | 433 | + | 0 | 1 | TGGGCTGTGAG |
| FAM290 | GT1GMSCAM4 | 439 | 444 | + | 0 | 1 | GAAAAA |
| FAM306 | ANAERO2CONSENSUS | 445 | 450 | − | 0 | 1 | AGCAGC |
| FAM003 | MYBPLANT | 480 | 490 | − | 0 | 1 | CACCAAACGGT |
| FAM325 | MYBCOREATCYCB1 | 481 | 485 | − | 0 | 1 | AACGG |
| FAM266 | MYB1AT | 492 | 497 | + | 0 | 1 | AAACCA |
| FAM099 | CCA1ATLHCB1 | 511 | 518 | + | 0 | 1 | AAAAATCT |
| FAM162 | LTRE1HVBLT49 | 544 | 549 | + | 0 | 1 | CCGAAA |
| FAM002 | SORLIP1AT | 545 | 557 | + | 0 | 1 | CGAAAAGCCACTA |
| FAM311 | EECCRCAH1 | 582 | 588 | + | 0 | 1 | GATTTGC |
| FAM311 | EECCRCAH1 | 596 | 602 | − | 0 | 1 | GACTTTC |
| FAM013 | DRE2COREZMRAB17 | 599 | 605 | − | 0 | 1 | ACCGACT |
| FAM290 | GT1GMSCAM4 | 611 | 616 | + | 0 | 1 | GAAAAA |
| FAM306 | ANAERO2CONSENSUS | 617 | 622 | − | 0 | 1 | AGCAGC |
| FAM010 | WBBOXPCWRKY1 | 675 | 689 | − | 0 | 1 | TTTGACTTTTGGCTT |
| FAM266 | MYB1AT | 687 | 692 | + | 0 | 1 | AAACCA |
| FAM003 | MYBPLANT | 688 | 698 | + | 0 | 1 | AACCAAACACA |
| FAM024 | 2SSEEDPROTBANAPA | 691 | 699 | + | 0 | 1 | CAAACACAC |
| FAM302 | SITEIIATCYTC | 715 | 725 | + | 0 | 1 | TGGGCCATTTA |
| FAM012 | IBOXCORE | 721 | 727 | − | 0 | 1 | GATAAAT |
| FAM311 | EECCRCAH1 | 738 | 744 | − | 0 | 1 | GATTTGC |
| FAM202 | -300ELEMENT | 748 | 756 | + | 0 | 1 | TGAAAAATT |
| FAM290 | GT1GMSCAM4 | 749 | 754 | + | 0 | 1 | GAAAAA |
| FAM270 | RAV1AAT | 758 | 762 | + | 0 | 1 | CAACA |
| FAM069 | SURECOREATSULTR11 | 800 | 806 | + | 0 | 1 | GAGACTA |
| FAM012 | IBOXCORE | 814 | 820 | + | 0 | 1 | GATAACT |
| FAM267 | NTBBF1ARROLB | 818 | 824 | + | 0 | 1 | ACTTTAT |
| FAM267 | TAAAGSTKST1 | 819 | 825 | − | 0 | 1 | TATAAAG |
| FAM307 | ANAERO3CONSENSUS | 828 | 834 | + | 0 | 1 | TCATCAC |
| FAM182 | OBP1ATGST6 | 893 | 903 | + | 0 | 1 | TACACTTTTGG |
| FAM302 | SITEIIATCYTC | 906 | 916 | + | 0 | 1 | TGGGCTCGGAG |
| FAM290 | GT1GMSCAM4 | 916 | 921 | + | 0 | 1 | GAAAAA |
| FAM304 | OSE2ROOTNODULE | 943 | 947 | + | 0 | 1 | CTCTT |
| FAM012 | IBOXCORE | 953 | 959 | + | 0 | 1 | GATAACA |
| FAM324 | CGCGBOXAT | 960 | 965 | + | 0 | 1 | ACGCGG |
| FAM324 | CGCGBOXAT | 960 | 965 | − | 0 | 1 | CCGCGT |
| FAM002 | SORLIP1AT | 967 | 979 | − | 0 | 1 | CGTTAGGCCACAT |
| FAM302 | SITEIIATCYTC | 984 | 994 | − | 0 | 1 | TGGGCCGGATT |
| FAM302 | UP1ATMSD | 988 | 998 | + | 0 | 1 | CGGCCCATTTA |
| FAM324 | CGCGBOXAT | 1018 | 1023 | + | 0 | 1 | ACGCGG |
| FAM324 | CGCGBOXAT | 1018 | 1023 | − | 0 | 1 | CCGCGT |
| FAM002 | RAV1BAT | 1023 | 1035 | − | 0 | 1 | TGGCACCTGCTCC |
| FAM010 | WBOXHVISO1 | 1028 | 1042 | − | 0 | 1 | AGTGACTTGGCACCT |
| FAM002 | RAV1BAT | 1051 | 1063 | − | 0 | 1 | CTCCACCTGCAGC |
| FAM151 | INTRONLOWER | 1053 | 1058 | + | 0 | 1 | TGCAGG |
| FAM263 | DPBFCOREDCDC3 | 1070 | 1076 | + | 0 | 1 | ACACTAG |
| FAM324 | CGCGBOXAT | 1079 | 1084 | + | 0 | 1 | CCGCGG |
| FAM324 | CGCGBOXAT | 1079 | 1084 | − | 0 | 1 | CCGCGG |
| FAM002 | GADOWNAT | 1090 | 1102 | − | 0 | 1 | CGACACGTGTCAG |
| FAM002 | GADOWNAT | 1091 | 1103 | + | 0 | 1 | TGACACGTGTCGC |
| FAM322 | BIHD1OS | 1091 | 1095 | − | 0 | 1 | TGTCA |
| FAM263 | DPBFCOREDCDC3 | 1093 | 1099 | + | 0 | 1 | ACACGTG |
| FAM263 | DPBFCOREDCDC3 | 1094 | 1100 | − | 0 | 1 | ACACGTG |
| FAM002 | SORLIP1AT | 1096 | 1108 | + | 0 | 1 | CGTGTCGCCACGT |
| FAM002 | ABREATRD2 | 1100 | 1112 | − | 0 | 1 | CGGCACGTGGCGA |
| FAM002 | GBOX10NT | 1101 | 1113 | + | 0 | 1 | CGCCACGTGCCGC |
| FAM061 | GCCCORE | 1108 | 1114 | + | 0 | 1 | TGCCGCC |
| FAM302 | SORLIP2AT | 1139 | 1149 | + | 0 | 1 | CGGGCCGACTG |
| FAM013 | DRECRTCOREAT | 1142 | 1148 | + | 0 | 1 | GCCGACT |
| FAM002 | TGACGTVMAMY | 1143 | 1155 | + | 0 | 1 | CCGACTGACGTCT |
| FAM002 | HEXMOTIFTAH3H4 | 1145 | 1157 | − | 0 | 1 | CAAGACGTCAGTC |
| FAM057 | ACGTCBOX | 1149 | 1154 | + | 0 | 1 | GACGTC |
| FAM057 | ACGTCBOX | 1149 | 1154 | − | 0 | 1 | GACGTC |
| FAM107 | CGACGOSAMY3 | 1172 | 1176 | + | 0 | 1 | CGACG |
| FAM061 | GCCCORE | 1179 | 1185 | − | 0 | 1 | CGCCGCC |
| FAM010 | ELRECOREPCRP1 | 1198 | 1212 | + | 0 | 1 | TTTGACCCCTCGCTA |
| FAM306 | ANAERO2CONSENSUS | 1236 | 1241 | − | 0 | 1 | AGCAGC |
| FAM002 | SORLIP1AT | 1274 | 1286 | + | 0 | 1 | CAGGACGCCACGT |
| FAM002 | ACGTABREMOTIFA2OSE | 1278 | 1290 | − | 0 | 1 | TTGGACGTGGCGT |
| FAM262 | CIACADIANLELHC | 1302 | 1311 | − | 0 | 1 | CAATGGCATC |
| FAM002 | SORLIP1AT | 1305 | 1317 | + | 0 | 1 | GCCATTGCCACCT |
| FAM324 | CGCGBOXAT | 1328 | 1333 | + | 0 | 1 | ACGCGT |
| FAM324 | CGCGBOXAT | 1328 | 1333 | − | 0 | 1 | ACGCGT |
| FAM010 | WBOXHVISO1 | 1337 | 1351 | + | 0 | 1 | CGTGACTATAAAAAA |
| FAM171 | MYBPZM | 1383 | 1389 | + | 0 | 1 | CCCTACC |
| FAM303 | OSE1ROOTNODULE | 1391 | 1397 | − | 0 | 1 | AAAGATT |
| FAM194 | PALBOXAPC | 1400 | 1406 | + | 0 | 1 | CCGTCCC |
| FAM302 | SITEIIATCYTC | 1405 | 1415 | − | 0 | 1 | TGGGCTGATGG |
| FAM311 | EECCRCAH1 | 1416 | 1422 | − | 0 | 1 | GAATTGC |
| FAM302 | SITEIIATCYTC | 1451 | 1461 | − | 0 | 1 | TGGGCTTCGGT |
| FAM013 | DRECRTCOREAT | 1466 | 1472 | + | 0 | 1 | GCCGACC |
| FAM003 | REALPHALGLHCB21 | 1482 | 1492 | − | 0 | 1 | AACCAACGGCA |
| FAM325 | MYBCOREATCYCB1 | 1484 | 1488 | − | 0 | 1 | AACGG |
| FAM194 | PALBOXAPC | 1493 | 1499 | + | 0 | 1 | CCGTCCC |
| FAM304 | OSE2ROOTNODULE | 1500 | 1504 | − | 0 | 1 | CTCTT |

2.) p-KG37

PLACE analysis results of p-KG37 are listed in Table 23, neither TATA box nor CAAT motifs are found in this promoter.

TABLE 23
PLACE analysis results of the 910bp promoter of p-KG37

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM311 | EECCRCAH1 | 6 | 12 | + | 0 | 1 | GATTTCC |
| FAM069 | SURECOREATSULTR11 | 26 | 32 | + | 0 | 1 | GAGACGA |
| FAM272 | SV40COREENHAN | 30 | 37 | − | 0 | 1 | GTGGTTCG |
| FAM202 | -300ELEMENT | 37 | 45 | − | 0 | 1 | TGAAAAATG |
| FAM290 | GT1GMSCAM4 | 39 | 44 | − | 0 | 1 | GAAAAA |
| FAM324 | CGCGBOXAT | 58 | 63 | + | 0 | 1 | GCGCGC |
| FAM324 | CGCGBOXAT | 58 | 63 | − | 0 | 1 | GCGCGC |
| FAM002 | ASF1MOTIFCAMV | 66 | 78 | − | 0 | 1 | GTCACTGACGATT |
| FAM271 | SEBFCONSSTPR10A | 74 | 80 | − | 0 | 1 | CTGTCAC |
| FAM322 | BIHD1OS | 75 | 79 | − | 0 | 1 | TGTCA |
| FAM008 | MYB2AT | 94 | 104 | + | 0 | 1 | TCCTTAACTGG |
| FAM281 | MYB1LEPR | 105 | 111 | − | 0 | 1 | GTTAGTT |
| FAM263 | DPBFCOREDCDC3 | 140 | 146 | + | 0 | 1 | ACACTGG |
| FAM273 | TATCCAOSAMY | 158 | 164 | − | 0 | 1 | TATCCAA |
| FAM014 | MYBST1 | 159 | 165 | + | 0 | 1 | TGGATAG |
| FAM325 | MYBCOREATCYCB1 | 175 | 179 | − | 0 | 1 | AACGG |
| FAM278 | UPRMOTIFIIAT | 180 | 198 | − | 0 | 1 | CCTTGCTTTTTAGCCCACG |
| FAM302 | SITEIIATCYTC | 182 | 192 | + | 0 | 1 | TGGGCTAAAAA |
| FAM002 | CACGTGMOTIF | 212 | 224 | − | 0 | 1 | GATCACGTGCGTT |
| FAM002 | CACGTGMOTIF | 213 | 225 | + | 0 | 1 | ACGCACGTGATCC |
| FAM069 | SURECOREATSULTR11 | 231 | 237 | − | 0 | 1 | GAGACCA |
| FAM266 | MYB1AT | 244 | 249 | + | 0 | 1 | AAACCA |
| FAM306 | ANAERO2CONSENSUS | 278 | 283 | − | 0 | 1 | AGCAGC |
| FAM002 | ASF1MOTIFCAMV | 285 | 297 | + | 0 | 1 | TCAGTTGACGGTG |
| FAM010 | WBOXATNPR1 | 288 | 302 | + | 0 | 1 | GTTGACGGTGTGCAC |
| FAM302 | SITEIIATCYTC | 337 | 347 | − | 0 | 1 | TGGGCTCCAAG |
| FAM205 | PYRIMIDINEBOXOSRAM | 351 | 356 | − | 0 | 1 | CCTTTT |
| FAM311 | EECCRCAH1 | 363 | 369 | − | 0 | 1 | GAATTTC |
| FAM325 | MYBCOREATCYCB1 | 387 | 391 | + | 0 | 1 | AACGG |
| FAM270 | RAV1AAT | 392 | 396 | + | 0 | 1 | CAACA |
| FAM205 | PYRIMIDINEBOXOSRAM | 396 | 401 | − | 0 | 1 | CCTTTT |
| FAM013 | LTRECOREATCOR15 | 405 | 411 | + | 0 | 1 | TCCGACA |
| FAM194 | PALBOXAPC | 446 | 452 | + | 0 | 1 | CCGTCCT |
| FAM263 | DPBFCOREDCDC3 | 477 | 483 | − | 0 | 1 | ACACTTG |
| FAM002 | SORLIP1AT | 479 | 491 | + | 0 | 1 | AGTGTTGCCACGC |
| FAM270 | RAV1AAT | 481 | 485 | − | 0 | 1 | CAACA |
| FAM324 | CGCGBOXAT | 491 | 496 | + | 0 | 1 | CCGCGC |
| FAM324 | CGCGBOXAT | 491 | 496 | − | 0 | 1 | GCGCGG |
| FAM002 | ASF1MOTIFCAMV | 554 | 566 | − | 0 | 1 | AGCAGTGACGCCG |
| FAM061 | GCCCORE | 578 | 584 | + | 0 | 1 | GGCCGCC |
| FAM002 | SORLIP1AT | 621 | 633 | + | 0 | 1 | ACCGAGGCCACCT |
| FAM205 | PYRIMIDINEBOXOSRAM | 631 | 636 | + | 0 | 1 | CCTTTT |
| FAM003 | REALPHALGLHCB21 | 633 | 643 | − | 0 | 1 | AACCAAGAAAA |
| FAM270 | RAV1AAT | 648 | 652 | + | 0 | 1 | CAACA |
| FAM002 | ABRELATERD | 677 | 689 | − | 0 | 1 | GCAGACGTGGTGC |
| FAM303 | OSE1ROOTNODULE | 703 | 709 | − | 0 | 1 | AAAGATT |
| FAM069 | SURECOREATSULTR11 | 745 | 751 | − | 0 | 1 | GAGACGG |
| FAM305 | ANAERO1CONSENSUS | 802 | 808 | − | 0 | 1 | AAACAAA |
| FAM194 | PALBOXAPC | 820 | 826 | + | 0 | 1 | CCGTCCT |
| FAM263 | DPBFCOREDCDC3 | 824 | 830 | − | 0 | 1 | ACACAGG |
| FAM325 | MYBCOREATCYCB1 | 859 | 863 | + | 0 | 1 | AACGG |
| FAM267 | TAAAGSTKST1 | 882 | 888 | − | 0 | 1 | CATAAAG |

3.) p-KG45

PLACE analysis results of p-KG45 are listed in Table 24, Three TATA Box motifs are found at nucleotide position 310-316, 312-318, and 1065-1071 of the forward strand, respectively. One CAAT Box motif is found at nucleotide position 976-980 of the forward strand that may be the functional motif working with the TATA box at position 1065-1071 to facilitate transcriptional initiation.

TABLE 24
PLACE analysis results of the 1131bp promoter of p-KG45

| | | | | | | | |
|---|---|---|---|---|---|---|---|
| IUPAC | | Start | End | | | | |
| Family | IUPAC | pos. | pos. | Strand | MisMatches | Score | Sequence |

| | | | | | | | |
|---|---|---|---|---|---|---|---|
| FAM276 | TRANSINITDICOTS | 4 | 11 | − | 0 | 1 | ACGATGGC |
| FAM263 | DPBFCOREDCDC3 | 33 | 39 | − | 0 | 1 | ACACACG |
| FAM263 | DPBFCOREDCDC3 | 49 | 55 | − | 0 | 1 | ACACACG |
| FAM228 | SEF3MOTIFGM | 74 | 79 | + | 0 | 1 | AACCCA |
| FAM171 | MYBPZM | 76 | 82 | + | 0 | 1 | CCCAACC |
| FAM003 | MYBPLANT | 79 | 89 | + | 0 | 1 | AACCAAACATC |
| FAM311 | EECCRCAH1 | 96 | 102 | + | 0 | 1 | GATTTCC |
| FAM234 | SP8BFIBSP8BIB | 122 | 128 | − | 0 | 1 | TACTATT |
| FAM310 | CPBCSPOR | 127 | 132 | + | 0 | 1 | TATTAG |
| FAM270 | RAV1AAT | 173 | 177 | + | 0 | 1 | CAACA |
| FAM242 | TATABOX3 | 177 | 183 | − | 0 | 1 | TATTAAT |
| FAM087 | BOXIINTPATPB | 184 | 189 | + | 0 | 1 | ATAGAA |
| FAM267 | NTBBF1ARROLB | 222 | 228 | + | 0 | 1 | ACTTTAT |
| FAM267 | TAAAGSTKST1 | 223 | 229 | − | 0 | 1 | AATAAAG |
| FAM290 | GT1GMSCAM4 | 229 | 234 | − | 0 | 1 | GAAAAA |
| FAM263 | DPBFCOREDCDC3 | 243 | 249 | − | 0 | 1 | ACACTGG |
| FAM234 | SP8BFIBSP8BIB | 254 | 260 | − | 0 | 1 | TACTATT |
| FAM290 | GT1GMSCAM4 | 276 | 281 | − | 0 | 1 | GAAAAA |
| FAM304 | OSE2ROOTNODULE | 288 | 292 | − | 0 | 1 | CTCTT |
| FAM295 | P1BS | 295 | 302 | + | 0 | 1 | GAATATTC |
| FAM295 | P1BS | 295 | 302 | − | 0 | 1 | GAATATTC |
| FAM205 | PYRIMIDINEBOXOSRAM | 304 | 309 | + | 0 | 1 | CCTTTT |
| FAM019 | TATAPVTRNALEU | 306 | 318 | − | 0 | 1 | ATTTATATAAAAA |
| FAM019 | TATAPVTRNALEU | 307 | 319 | + | 0 | 1 | TTTTATATAAATT |
| FAM243 | TATABOX4 | 309 | 315 | − | 0 | 1 | TATATAA |
| FAM243 | TATABOX4 | 310 | 316 | + | 0 | 1 | TATATAA |
| FAM241 | TATABOX2 | 312 | 318 | + | 0 | 1 | TATAAAT |
| FAM027 | -10PEHVPSBD | 323 | 328 | + | 0 | 1 | TATTCT |
| FAM002 | ASF1MOTIFCAMV | 332 | 344 | − | 0 | 1 | GTGTGTGACGCTT |
| FAM263 | DPBFCOREDCDC3 | 339 | 345 | + | 0 | 1 | ACACACG |
| FAM267 | TAAAGSTKST1 | 351 | 357 | + | 0 | 1 | CCTAAAG |
| FAM303 | OSE1ROOTNODULE | 354 | 360 | + | 0 | 1 | AAAGATA |
| FAM027 | -10PEHVPSBD | 359 | 364 | + | 0 | 1 | TATTCT |
| FAM270 | RAV1AAT | 372 | 376 | + | 0 | 1 | CAACA |
| FAM263 | DPBFCOREDCDC3 | 374 | 380 | + | 0 | 1 | ACACAAG |
| FAM202 | -300ELEMENT | 386 | 394 | + | 0 | 1 | TGAAAAGGT |
| FAM205 | PYRIMIDINEBOXOSRAM | 388 | 393 | − | 0 | 1 | CCTTTT |
| FAM270 | RAV1AAT | 413 | 417 | − | 0 | 1 | CAACA |
| FAM275 | TGTCACACMCUCUMISIN | 426 | 432 | − | 0 | 1 | TGTCACA |
| FAM322 | BIHD1OS | 428 | 432 | − | 0 | 1 | TGTCA |
| FAM103 | CELLCYCLESC | 431 | 438 | + | 0 | 1 | CACGAAAA |
| FAM267 | TAAAGSTKST1 | 439 | 445 | + | 0 | 1 | TTTAAAG |
| FAM289 | LEAFYATAG | 461 | 467 | − | 0 | 1 | CCAATGT |
| FAM100 | CCAATBOX1 | 463 | 467 | − | 0 | 1 | CCAAT |
| FAM021 | GT1CORE | 484 | 494 | − | 0 | 1 | TGGTTAATATG |
| FAM266 | MYB1AT | 489 | 494 | + | 0 | 1 | TAACCA |
| FAM003 | REALPHALGLHCB21 | 490 | 500 | + | 0 | 1 | AACCAACTATT |
| FAM310 | CPBCSPOR | 497 | 502 | + | 0 | 1 | TATTAG |
| FAM169 | MYBATRD2 | 530 | 536 | − | 0 | 1 | CTAACCA |
| FAM266 | MYB1AT | 530 | 535 | − | 0 | 1 | TAACCA |
| FAM087 | BOXIINTPATPB | 558 | 563 | − | 0 | 1 | ATAGAA |
| FAM170 | AMYBOX1 | 597 | 603 | − | 0 | 1 | TAACAGA |
| FAM278 | UPRMOTIFIIAT | 628 | 646 | + | 0 | 1 | CCAAATGTATAATCCCACG |
| FAM172 | MYCATRD2 | 652 | 658 | − | 0 | 1 | CACATGA |
| FAM172 | MYCATERD | 653 | 659 | + | 0 | 1 | CATGTGA |
| FAM010 | WBOXHVISO1 | 655 | 669 | + | 0 | 1 | TGTGACTCCATTTCG |
| FAM002 | ABRELATERD | 740 | 752 | − | 0 | 1 | AGATACGTGAACG |
| FAM263 | DPBFCOREDCDC3 | 751 | 757 | − | 0 | 1 | ACACAAG |
| FAM024 | CANBNNAPA | 752 | 760 | − | 0 | 1 | CGAACACAA |
| FAM325 | MYBCOREATCYCB1 | 771 | 775 | − | 0 | 1 | AACGG |
| FAM002 | RAV1BAT | 830 | 842 | − | 0 | 1 | CATCACCTGCCTC |
| FAM307 | ANAERO3CONSENSUS | 837 | 843 | − | 0 | 1 | TCATCAC |
| FAM322 | BIHD1OS | 841 | 845 | − | 0 | 1 | TGTCA |
| FAM263 | DPBFCOREDCDC3 | 843 | 849 | + | 0 | 1 | ACACGCG |
| FAM324 | CGCGBOXAT | 845 | 850 | + | 0 | 1 | ACGCGC |
| FAM324 | CGCGBOXAT | 845 | 850 | − | 0 | 1 | GCGCGT |
| FAM302 | SORLIP2AT | 855 | 865 | + | 0 | 1 | CGGGCCGATGC |
| FAM013 | DRECRTCOREAT | 864 | 870 | + | 0 | 1 | GCCGACG |
| FAM002 | SORLIP1AT | 866 | 878 | + | 0 | 1 | CGACGCGCCACCG |
| FAM107 | CGACGOSAMY3 | 866 | 870 | + | 0 | 1 | CGACG |
| FAM324 | CGCGBOXAT | 868 | 873 | + | 0 | 1 | ACGCGC |
| FAM324 | CGCGBOXAT | 868 | 873 | − | 0 | 1 | GCGCGT |
| FAM306 | ANAERO2CONSENSUS | 901 | 906 | + | 0 | 1 | AGCAGC |
| FAM002 | ABRELATERD | 912 | 924 | + | 0 | 1 | AGAGACGTGGAGC |
| FAM050 | ABREBZMRAB28 | 913 | 922 | − | 0 | 1 | TCCACGTCTC |
| FAM069 | SURECOREATSULTR11 | 913 | 919 | + | 0 | 1 | GAGACGT |
| FAM069 | SURECOREATSULTR11 | 931 | 937 | + | 0 | 1 | GAGACTT |
| FAM267 | NTBBF1ARROLB | 934 | 940 | + | 0 | 1 | ACTTTAG |
| FAM267 | TAAAGSTKST1 | 935 | 941 | − | 0 | 1 | CCTAAAG |
| FAM069 | SURECOREATSULTR11 | 948 | 954 | + | 0 | 1 | GAGACCA |
| FAM322 | BIHD1OS | 968 | 972 | − | 0 | 1 | TGTCA |
| FAM278 | UPRMOTIFIIAT | 975 | 993 | + | 0 | 1 | CCCAATGATCAGGACCACG |
| FAM100 | CCAATBOX1 | 976 | 980 | + | 0 | 1 | CCAAT |
| FAM002 | CACGTGMOTIF | 994 | 1006 | − | 0 | 1 | TGACACGTGCAAG |
| FAM002 | GADOWNAT | 995 | 1007 | + | 0 | 1 | TTGCACGTGTCAG |
| FAM263 | DPBFCOREDCDC3 | 998 | 1004 | − | 0 | 1 | ACACGTG |
| FAM002 | RAV1BAT | 1001 | 1013 | − | 0 | 1 | AGGCACCTGACAC |
| FAM322 | BIHD1OS | 1002 | 1006 | + | 0 | 1 | TGTCA |
| FAM324 | CGCGBOXAT | 1021 | 1026 | + | 0 | 1 | ACGCGT |
| FAM324 | CGCGBOXAT | 1021 | 1026 | − | 0 | 1 | ACGCGT |
| FAM107 | CGACGOSAMY3 | 1024 | 1028 | − | 0 | 1 | CGACG |
| FAM324 | CGCGBOXAT | 1028 | 1033 | + | 0 | 1 | GCGCGC |
| FAM324 | CGCGBOXAT | 1028 | 1033 | − | 0 | 1 | GCGCGC |
| FAM322 | BIHD1OS | 1047 | 1051 | + | 0 | 1 | TGTCA |
| FAM107 | CGACGOSAMY3 | 1059 | 1063 | + | 0 | 1 | CGACG |
| FAM019 | TATAPVTRNALEU | 1061 | 1073 | − | 0 | 1 | CTTTATATAGCGT |
| FAM243 | TATABOX4 | 1065 | 1071 | + | 0 | 1 | TATATAA |
| FAM267 | TAAAGSTKST1 | 1067 | 1073 | + | 0 | 1 | TATAAAG |
| FAM267 | NTBBF1ARROLB | 1068 | 1074 | − | 0 | 1 | ACTTTAT |
| FAM272 | SV40COREENHAN | 1073 | 1080 | + | 0 | 1 | GTGGTAAG |
| FAM302 | SORLIP2AT | 1094 | 1104 | + | 0 | 1 | GGGGCCGCCCC |
| FAM061 | GCCCORE | 1096 | 1102 | + | 0 | 1 | GGCCGCC |
| FAM302 | SORLIP2AT | 1103 | 1113 | − | 0 | 1 | AGGGCCGTTGG |
| FAM325 | MYBCOREATCYCB1 | 1105 | 1109 | + | 0 | 1 | AACGG |
| FAM278 | UPRMOTIFIIAT | 1111 | 1129 | + | 0 | 1 | CCTTGCGATCGCCACCACG |
| FAM002 | SORLIP1AT | 1115 | 1127 | + | 0 | 1 | GCGATCGCCACCA |

4.) p-KG46

PLACE analysis results of p-KG46 are listed in Table 25, neither TATA box nor CAAT motifs are found in this promoter.

TABLE 25
PLACE analysis results of the 563bp promoter of p-KG46

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM002 | SORLIP1AT | 40 | 52 | − | 0 | 1 | TAAATTGCCACCC |
| FAM170 | AMYBOX1 | 54 | 60 | + | 0 | 1 | TAACAGA |
| FAM311 | EECCRCAH1 | 59 | 65 | + | 0 | 1 | GAATTGC |
| FAM292 | PREATPRODH | 69 | 74 | − | 0 | 1 | ACTCAT |
| FAM271 | SEBFCONSSTPR10A | 121 | 127 | + | 0 | 1 | TTGTCAC |
| FAM275 | TGTCACACMCUCUMISIN | 122 | 128 | + | 0 | 1 | TGTCACA |
| FAM322 | BIHD1OS | 122 | 126 | + | 0 | 1 | TGTCA |
| FAM172 | MYCATERD | 124 | 130 | − | 0 | 1 | CATGTGA |
| FAM172 | MYCATRD2 | 125 | 131 | + | 0 | 1 | CACATGT |
| FAM172 | MYCATRD2 | 126 | 132 | − | 0 | 1 | CACATGT |
| FAM172 | MYCATERD | 127 | 133 | + | 0 | 1 | CATGTGG |
| FAM002 | SORLIP1AT | 128 | 140 | − | 0 | 1 | AAAAAGGCCACAT |
| FAM205 | PYRIMIDINEBOXOSRAM | 134 | 139 | + | 0 | 1 | CCTTTT |
| FAM003 | REALPHALGLHCB21 | 135 | 145 | − | 0 | 1 | AACCAAAAAAG |
| FAM302 | SITEIIATCYTC | 171 | 181 | + | 0 | 1 | TGGGCTGTCAT |
| FAM322 | BIHD1OS | 176 | 180 | + | 0 | 1 | TGTCA |
| FAM304 | OSE2ROOTNODULE | 203 | 207 | + | 0 | 1 | CTCTT |
| FAM012 | IBOXCORE | 212 | 218 | − | 0 | 1 | GATAATG |
| FAM002 | ASF1MOTIFCAMV | 229 | 241 | − | 0 | 1 | GGAAATGACGATG |
| FAM069 | SURECOREATSULTR11 | 245 | 251 | + | 0 | 1 | GAGACCC |
| FAM322 | BIHD1OS | 260 | 264 | + | 0 | 1 | TGTCA |
| FAM263 | DPBFCOREDCDC3 | 277 | 283 | + | 0 | 1 | ACACGCG |
| FAM324 | CGCGBOXAT | 279 | 284 | + | 0 | 1 | ACGCGT |
| FAM324 | CGCGBOXAT | 279 | 284 | − | 0 | 1 | ACGCGT |
| FAM107 | CGACGOSAMY3 | 282 | 286 | − | 0 | 1 | CGACG |
| FAM107 | CGACGOSAMY3 | 287 | 291 | − | 0 | 1 | CGACG |
| FAM002 | RAV1BAT | 294 | 306 | − | 0 | 1 | ACCCACCTGGCCT |
| FAM002 | SITEIOSPCNA | 295 | 307 | + | 0 | 1 | GGCCAGGTGGGTT |
| FAM228 | SEF3MOTIFGM | 302 | 307 | − | 0 | 1 | AACCCA |
| FAM194 | PALBOXAPC | 354 | 360 | + | 0 | 1 | CCGTCCA |
| FAM194 | CMSRE1IBSPOA | 354 | 360 | − | 0 | 1 | TGGACGG |
| FAM013 | DRE2COREZMRAB17 | 360 | 366 | + | 0 | 1 | ACCGACT |
| FAM026 | RYREPEATLEGUMINBOX | 393 | 403 | + | 0 | 1 | ACCATGCACGA |
| FAM107 | CGACGOSAMY3 | 401 | 405 | + | 0 | 1 | CGACG |
| FAM002 | GADOWNAT | 403 | 415 | − | 0 | 1 | TCGCACGTGTCGT |
| FAM002 | CACGTGMOTIF | 404 | 416 | + | 0 | 1 | CGACACGTGCGAT |
| FAM047 | ABRE2HVA22 | 405 | 414 | − | 0 | 1 | CGCACGTGTC |
| FAM263 | DPBFCOREDCDC3 | 406 | 412 | + | 0 | 1 | ACACGTG |
| FAM002 | RAV1BAT | 433 | 445 | + | 0 | 1 | ACTCACCTGTTGC |
| FAM270 | RAV1AAT | 440 | 444 | − | 0 | 1 | CAACA |
| FAM014 | MYBST1 | 450 | 456 | − | 0 | 1 | TGGATAT |
| FAM025 | TATCCAYMOTIFOSRAMY | 451 | 457 | + | 0 | 1 | TATCCAC |
| FAM273 | TATCCACHVAL21 | 451 | 457 | + | 0 | 1 | TATCCAC |
| FAM010 | WBOXNTCHN48 | 502 | 516 | + | 0 | 1 | GCTGACCAGAGAGCT |

5.) p-KG49

PLACE analysis results of p-KG49 is listed in Table 26, One TATA Box motif is found at nucleotide position 803-809 of the forward strand and one CAAT Box motif is found at nucleotide position 472-476 at the reverse strand.

TABLE 26
PLACE analysis results of the 1188bp promoter of p-KG49

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM013 | DRECRTCOREAT | 29 | 35 | − | 0 | 1 | GCCGACA |
| FAM324 | CGCGBOXAT | 45 | 50 | + | 0 | 1 | CCGCGT |
| FAM324 | CGCGBOXAT | 45 | 50 | − | 0 | 1 | ACGCGG |
| FAM324 | CGCGBOXAT | 57 | 62 | + | 0 | 1 | CCGCGG |
| FAM324 | CGCGBOXAT | 57 | 62 | − | 0 | 1 | CCGCGG |
| FAM069 | SURECOREATSULTR11 | 60 | 66 | − | 0 | 1 | GAGACCG |
| FAM107 | CGACGOSAMY3 | 68 | 72 | − | 0 | 1 | CGACG |
| FAM069 | SURECOREATSULTR11 | 70 | 76 | − | 0 | 1 | GAGACGA |
| FAM272 | SV40COREENHAN | 101 | 108 | − | 0 | 1 | GTGGAAAG |
| FAM278 | UPRMOTIFIIAT | 126 | 144 | + | 0 | 1 | CCACCCCCTTCTCCCCACG |
| FAM324 | CGCGBOXAT | 159 | 164 | + | 0 | 1 | ACGCGC |
| FAM324 | CGCGBOXAT | 159 | 164 | − | 0 | 1 | GCGCGT |
| FAM270 | RAV1AAT | 173 | 177 | − | 0 | 1 | CAACA |
| FAM307 | ANAERO3CONSENSUS | 179 | 185 | + | 0 | 1 | TCATCAC |
| FAM002 | ASF1MOTIFCAMV | 196 | 208 | − | 0 | 1 | CTCTGTGACGCTT |
| FAM147 | HEXAMERATH4 | 231 | 236 | + | 0 | 1 | CCGTCG |
| FAM107 | CGACGOSAMY3 | 232 | 236 | − | 0 | 1 | CGACG |
| FAM147 | HEXAMERATH4 | 237 | 242 | + | 0 | 1 | CCGTCG |
| FAM107 | CGACGOSAMY3 | 238 | 242 | − | 0 | 1 | CGACG |
| FAM152 | INTRONUPPER | 249 | 257 | + | 0 | 1 | CAGGTAAGT |
| FAM010 | WBOXHVISO1 | 258 | 272 | + | 0 | 1 | AATGACTAATCGCCT |
| FAM069 | SURECOREATSULTR11 | 273 | 279 | − | 0 | 1 | GAGACTC |
| FAM266 | MYB1AT | 303 | 308 | − | 0 | 1 | AAACCA |
| FAM013 | LTRECOREATCOR15 | 313 | 319 | − | 0 | 1 | TCCGACT |
| FAM057 | ACGTCBOX | 318 | 323 | + | 0 | 1 | GACGTC |
| FAM057 | ACGTCBOX | 318 | 323 | − | 0 | 1 | GACGTC |
| FAM013 | LTRECOREATCOR15 | 320 | 326 | − | 0 | 1 | TCCGACG |
| FAM107 | CGACGOSAMY3 | 320 | 324 | − | 0 | 1 | CGACG |
| FAM002 | SORLIP1AT | 378 | 390 | + | 0 | 1 | TTCGACGCCACAT |
| FAM107 | CGACGOSAMY3 | 380 | 384 | + | 0 | 1 | CGACG |
| FAM290 | GT1GMSCAM4 | 392 | 397 | − | 0 | 1 | GAAAAA |
| FAM171 | MYBPZM | 404 | 410 | + | 0 | 1 | GCCAACC |
| FAM324 | CGCGBOXAT | 414 | 419 | + | 0 | 1 | GCGCGT |
| FAM324 | CGCGBOXAT | 414 | 419 | − | 0 | 1 | ACGCGC |
| FAM025 | TATCCAYMOTIFOSRAMY | 466 | 472 | − | 0 | 1 | TATCCAC |
| FAM273 | TATCCACHVAL21 | 466 | 472 | − | 0 | 1 | TATCCAC |
| FAM014 | MYBST1 | 467 | 473 | + | 0 | 1 | TGGATAT |
| FAM100 | CCAATBOX1 | 472 | 476 | − | 0 | 1 | CCAAT |
| FAM003 | REALPHALGLHCB21 | 479 | 489 | − | 0 | 1 | AACCAAAAAAA |
| FAM169 | MYBATRD2 | 485 | 491 | − | 0 | 1 | CTAACCA |
| FAM266 | MYB1AT | 485 | 490 | − | 0 | 1 | TAACCA |
| FAM270 | RAV1AAT | 534 | 538 | − | 0 | 1 | CAACA |
| FAM170 | MYBGAHV | 546 | 552 | − | 0 | 1 | TAACAAA |
| FAM177 | NRRBNEXTA | 551 | 558 | + | 0 | 1 | TAGTGGAT |
| FAM069 | SURECOREATSULTR11 | 629 | 635 | + | 0 | 1 | GAGACTA |
| FAM069 | SURECOREATSULTR11 | 647 | 653 | + | 0 | 1 | GAGACTA |
| FAM010 | ELRECOREPCRP1 | 653 | 667 | − | 0 | 1 | CTTGACCATTCGCAT |
| FAM311 | EECCRCAH1 | 669 | 675 | + | 0 | 1 | GAATTTC |
| FAM003 | REALPHALGLHCB21 | 676 | 686 | − | 0 | 1 | AACCAAGGCGA |
| FAM266 | MYB1AT | 682 | 687 | − | 0 | 1 | AAACCA |
| FAM317 | SORLREP3AT | 722 | 730 | + | 0 | 1 | TGTATATAT |
| FAM266 | MYB1AT | 732 | 737 | − | 0 | 1 | AAACCA |
| FAM002 | T/GBOXATPIN2 | 740 | 752 | − | 0 | 1 | CTAAACGTGCCGA |
| FAM322 | BIHD1OS | 781 | 785 | + | 0 | 1 | TGTCA |
| FAM322 | BIHD1OS | 792 | 796 | + | 0 | 1 | TGTCA |
| FAM243 | TATABOX4 | 803 | 809 | + | 0 | 1 | TATATAA |
| FAM010 | WBOXATNPR1 | 833 | 847 | + | 0 | 1 | ATTGACTTATTATGC |
| FAM311 | EECCRCAH1 | 846 | 852 | − | 0 | 1 | GACTTGC |
| FAM021 | GT1CORE | 851 | 861 | − | 0 | 1 | AGGTTAATCGA |
| FAM302 | SITEIIATCYTC | 865 | 875 | + | 0 | 1 | TGGGCTCAGTG |
| FAM221 | S1FBOXSORPS1L21 | 879 | 884 | − | 0 | 1 | ATGGTA |
| FAM010 | WBOXNTCHN48 | 945 | 959 | − | 0 | 1 | GCTGACTAGCCGAGT |
| FAM321 | WRECSAA01 | 982 | 991 | − | 0 | 1 | AAAGTATCGA |
| FAM069 | ARFAT | 997 | 1003 | + | 0 | 1 | ATGTCTC |
| FAM069 | SURECOREATSULTR11 | 997 | 1003 | − | 0 | 1 | GAGACAT |
| FAM021 | GT1CORE | 1007 | 1017 | − | 0 | 1 | TGGTTAACACA |
| FAM266 | MYB1AT | 1012 | 1017 | + | 0 | 1 | TAACCA |
| FAM002 | SORLIP1AT | 1020 | 1032 | + | 0 | 1 | GTGTGTGCCACAT |
| FAM039 | AACACOREOSGLUB1 | 1051 | 1057 | − | 0 | 1 | AACAAAC |
| FAM021 | GT1CORE | 1052 | 1062 | − | 0 | 1 | AGGTTAACAAA |
| FAM170 | MYBGAHV | 1052 | 1058 | − | 0 | 1 | TAACAAA |
| FAM329 | XYLAT | 1125 | 1132 | − | 0 | 1 | ACAAAGAA |

6.) p-KG56

PLACE analysis results of p-KG56 are listed in Table 27. Two TATA Box motifs are found at nucleotide position 729-735, and 1900-1906 of the forward strand respectively. One CAAT Box motif is found at nucleotide position 599-603 of the reverse strand.

TABLE 27
PLACE analysis results of the 1188bp promoter of p-KG56

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM010 | WBOXNTCHN48 | 7 | 21 | − | 0 | 1 | GCTGACCAGATGTTC |
| FAM267 | TAAAGSTKST1 | 33 | 39 | + | 0 | 1 | GGTAAAG |
| FAM027 | -10PEHVPSBD | 47 | 52 | + | 0 | 1 | TATTCT |
| FAM329 | XYLAT | 62 | 69 | − | 0 | 1 | ACAAAGAA |
| FAM012 | IBOXCORE | 155 | 161 | + | 0 | 1 | GATAATG |
| FAM270 | RAV1AAT | 165 | 169 | + | 0 | 1 | CAACA |
| FAM021 | GT1CORE | 170 | 180 | − | 0 | 1 | CGGTTAATCTC |
| FAM026 | RYREPEATGMGY2 | 201 | 211 | + | 0 | 1 | ATCATGCATTA |
| FAM267 | TAAAGSTKST1 | 208 | 214 | + | 0 | 1 | ATTAAAG |
| FAM267 | NTBBF1ARROLB | 209 | 215 | − | 0 | 1 | ACTTTAA |
| FAM171 | MYBPZM | 216 | 222 | + | 0 | 1 | TCCTACC |
| FAM170 | GARE2OSREP1 | 296 | 302 | − | 0 | 1 | TAACGTA |
| FAM012 | IBOXCORE | 304 | 310 | − | 0 | 1 | GATAATT |
| FAM014 | SREATMSD | 305 | 311 | + | 0 | 1 | ATTATCC |
| FAM014 | MYBST1 | 306 | 312 | − | 0 | 1 | AGGATAA |
| FAM205 | PYRIMIDINEBOXOSRAM | 310 | 315 | + | 0 | 1 | CCTTTT |
| FAM014 | MYBST1 | 362 | 368 | − | 0 | 1 | TGGATAG |
| FAM273 | TATCCAOSAMY | 363 | 369 | + | 0 | 1 | TATCCAG |
| FAM002 | ASF1MOTIFCAMV | 379 | 391 | + | 0 | 1 | GAAGTTGACGCTC |
| FAM010 | WBOXATNPR1 | 382 | 396 | + | 0 | 1 | GTTGACGCTCTCAAA |
| FAM245 | TBOXATGAPB | 393 | 398 | − | 0 | 1 | ACTTTG |
| FAM010 | WBOXATNPR1 | 416 | 430 | + | 0 | 1 | ATTGACACATTTTTT |
| FAM322 | BIHD1OS | 418 | 422 | − | 0 | 1 | TGTCA |
| FAM267 | TAAAGSTKST1 | 436 | 442 | + | 0 | 1 | CTTAAAG |
| FAM304 | OSE2ROOTNODULE | 445 | 449 | + | 0 | 1 | CTCTT |
| FAM002 | RAV1BAT | 447 | 459 | + | 0 | 1 | CTTCACCTGAGAT |
| FAM202 | -300ELEMENT | 461 | 469 | + | 0 | 1 | TGAAAAAGG |
| FAM290 | GT1GMSCAM4 | 462 | 467 | + | 0 | 1 | GAAAAA |
| FAM205 | PYRIMIDINEBOXOSRAM | 464 | 469 | − | 0 | 1 | CCTTTT |
| FAM171 | BOXLCOREDCPAL | 469 | 475 | − | 0 | 1 | ACCATCC |
| FAM263 | DPBFCOREDCDC3 | 504 | 510 | + | 0 | 1 | ACACGGG |
| FAM061 | AGCBOXNPGLB | 527 | 533 | − | 0 | 1 | AGCCGCC |
| FAM002 | ASF1MOTIFCAMV | 563 | 575 | + | 0 | 1 | CAAGGTGACGCGG |
| FAM324 | CGCGBOXAT | 570 | 575 | + | 0 | 1 | ACGCGG |
| FAM324 | CGCGBOXAT | 570 | 575 | − | 0 | 1 | CCGCGT |
| FAM170 | AMYBOX1 | 591 | 597 | − | 0 | 1 | TAACAGA |
| FAM289 | LEAFYATAG | 597 | 603 | − | 0 | 1 | CCAATGT |
| FAM100 | CCAATBOX1 | 599 | 603 | − | 0 | 1 | CCAAT |
| FAM013 | DRE2COREZMRAB17 | 635 | 641 | + | 0 | 1 | ACCGACA |
| FAM163 | LTREATLTI78 | 635 | 641 | + | 0 | 1 | ACCGACA |
| FAM324 | CGCGBOXAT | 650 | 655 | + | 0 | 1 | ACGCGC |
| FAM324 | CGCGBOXAT | 650 | 655 | − | 0 | 1 | GCGCGT |
| FAM325 | MYBCOREATCYCB1 | 669 | 673 | − | 0 | 1 | AACGG |
| FAM069 | SURECOREATSULTR11 | 681 | 687 | + | 0 | 1 | GAGACTT |
| FAM290 | GT1GMSCAM4 | 691 | 696 | − | 0 | 1 | GAAAAA |
| FAM290 | GT1GMSCAM4 | 700 | 705 | + | 0 | 1 | GAAAAA |
| FAM241 | TATABOX2 | 729 | 735 | + | 0 | 1 | TATAAAT |
| FAM304 | OSE2ROOTNODULE | 736 | 740 | + | 0 | 1 | CTCTT |
| FAM162 | LTRE1HVBLT49 | 755 | 760 | + | 0 | 1 | CCGAAA |
| FAM311 | EECCRCAH1 | 780 | 786 | + | 0 | 1 | GATTTTC |
| FAM304 | OSE2ROOTNODULE | 786 | 790 | + | 0 | 1 | CTCTT |
| FAM002 | SORLIP1AT | 795 | 807 | + | 0 | 1 | GTATCTGCCACGC |
| FAM002 | SORLIP1AT | 821 | 833 | + | 0 | 1 | TCTATGGCCACTG |
| FAM304 | OSE2ROOTNODULE | 843 | 847 | − | 0 | 1 | CTCTT |
| FAM304 | OSE2ROOTNODULE | 865 | 869 | + | 0 | 1 | CTCTT |
| FAM263 | DPBFCOREDCDC3 | 910 | 916 | + | 0 | 1 | ACACGAG |
| FAM107 | CGACGOSAMY3 | 932 | 936 | − | 0 | 1 | CGACG |
| FAM263 | DPBFCOREDCDC3 | 945 | 951 | − | 0 | 1 | ACACTTG |
| FAM061 | GCCCORE | 954 | 960 | − | 0 | 1 | TGCCGCC |
| FAM171 | MYBPZM | 993 | 999 | + | 0 | 1 | CCCAACC |
| FAM171 | MYBPZM | 1001 | 1007 | − | 0 | 1 | GCCTACC |
| FAM010 | WBOXATNPR1 | 1023 | 1037 | − | 0 | 1 | CTTGACACAATCTGA |
| FAM322 | BIHD1OS | 1031 | 1035 | + | 0 | 1 | TGTCA |
| FAM008 | MYB2AT | 1047 | 1057 | + | 0 | 1 | GTGATAACTGA |
| FAM012 | IBOXCORE | 1049 | 1055 | + | 0 | 1 | GATAACT |
| FAM002 | SORLIP1AT | 1055 | 1067 | + | 0 | 1 | TGATAAGCCACTG |
| FAM012 | IBOX | 1056 | 1062 | + | 0 | 1 | GATAAGC |
| FAM002 | RAV1BAT | 1084 | 1096 | − | 0 | 1 | CGTCACCTGCAGC |
| FAM151 | INTRONLOWER | 1086 | 1091 | + | 0 | 1 | TGCAGG |
| FAM002 | ASF1MOTIFCAMV | 1087 | 1099 | + | 0 | 1 | GCAGGTGACGAAG |
| FAM324 | CGCGBOXAT | 1113 | 1118 | + | 0 | 1 | GCGCGG |
| FAM324 | CGCGBOXAT | 1113 | 1118 | − | 0 | 1 | CCGCGC |
| FAM325 | MYBCOREATCYCB1 | 1130 | 1134 | − | 0 | 1 | AACGG |
| FAM061 | GCCCORE | 1137 | 1143 | − | 0 | 1 | TGCCGCC |
| FAM002 | CACGTGMOTIF | 1138 | 1150 | − | 0 | 1 | CGTCACGTGCCGC |
| FAM002 | CACGTGMOTIF | 1139 | 1151 | + | 0 | 1 | CGGCACGTGACGA |
| FAM002 | ASF1MOTIFCAMV | 1141 | 1153 | + | 0 | 1 | GCACGTGACGAGC |
| FAM061 | GCCCORE | 1156 | 1162 | − | 0 | 1 | CGCCGCC |
| FAM311 | EECCRCAH1 | 1166 | 1172 | + | 0 | 1 | GACTTCC |
| FAM263 | DPBFCOREDCDC3 | 1172 | 1178 | − | 0 | 1 | ACACCAG |
| FAM013 | LTRECOREATCOR15 | 1185 | 1191 | − | 0 | 1 | TCCGACC |
| FAM324 | CGCGBOXAT | 1191 | 1196 | + | 0 | 1 | ACGCGT |
| FAM324 | CGCGBOXAT | 1191 | 1196 | − | 0 | 1 | ACGCGT |
| FAM002 | ASF1MOTIFCAMV | 1198 | 1210 | + | 0 | 1 | CGTGATGACGCAC |
| FAM307 | ANAERO3CONSENSUS | 1199 | 1205 | − | 0 | 1 | TCATCAC |
| FAM061 | GCCCORE | 1211 | 1217 | − | 0 | 1 | TGCCGCC |
| FAM262 | CIACADIANLELHC | 1225 | 1234 | + | 0 | 1 | CAAACTCATC |
| FAM292 | PREATPRODH | 1228 | 1233 | + | 0 | 1 | ACTCAT |
| FAM324 | CGCGBOXAT | 1241 | 1246 | + | 0 | 1 | GCGCGC |
| FAM324 | CGCGBOXAT | 1241 | 1246 | − | 0 | 1 | GCGCGC |
| FAM324 | CGCGBOXAT | 1251 | 1256 | + | 0 | 1 | ACGCGG |
| FAM324 | CGCGBOXAT | 1251 | 1256 | − | 0 | 1 | CCGCGT |
| FAM013 | DRE2COREZMRAB17 | 1269 | 1275 | − | 0 | 1 | ACCGACT |
| FAM013 | LTRECOREATCOR15 | 1280 | 1286 | + | 0 | 1 | CCCGACA |
| FAM302 | SORLIP2AT | 1287 | 1297 | + | 0 | 1 | AGGGCCTCATG |
| FAM013 | LTRECOREATCOR15 | 1316 | 1322 | + | 0 | 1 | CCCGACA |
| FAM302 | SITEIIATCYTC | 1323 | 1333 | + | 0 | 1 | TGGGCTGGGCC |
| FAM302 | SITEIIATCYTC | 1328 | 1338 | + | 0 | 1 | TGGGCCTCCTT |
| FAM057 | ACGTCBOX | 1346 | 1351 | + | 0 | 1 | GACGTC |
| FAM057 | ACGTCBOX | 1346 | 1351 | − | 0 | 1 | GACGTC |
| FAM107 | CGACGOSAMY3 | 1348 | 1352 | − | 0 | 1 | CGACG |
| FAM002 | ASF1MOTIFCAMV | 1351 | 1363 | − | 0 | 1 | GTGCGTGACGACG |
| FAM107 | CGACGOSAMY3 | 1351 | 1355 | − | 0 | 1 | CGACG |
| FAM194 | PALBOXAPC | 1370 | 1376 | − | 0 | 1 | CCGTCCT |
| FAM302 | SORLIP2AT | 1374 | 1384 | + | 0 | 1 | CGGGCCTCCCC |
| FAM302 | SITEIIATCYTC | 1381 | 1391 | − | 0 | 1 | TGGGCTCGGGG |
| FAM171 | BOXLCOREDCPAL | 1391 | 1397 | + | 0 | 1 | ACCTTCC |
| FAM089 | BS1EGCCR | 1420 | 1425 | − | 0 | 1 | AGCGGG |
| FAM322 | BIHD1OS | 1425 | 1429 | − | 0 | 1 | TGTCA |
| FAM026 | RYREPEATBNNAPA | 1430 | 1440 | − | 0 | 1 | CACATGCAGGG |
| FAM151 | INTRONLOWER | 1431 | 1436 | − | 0 | 1 | TGCAGG |
| FAM172 | MYCATRD2 | 1434 | 1440 | − | 0 | 1 | CACATGC |
| FAM172 | MYCATERD | 1435 | 1441 | + | 0 | 1 | CATGTGC |
| FAM151 | INTRONLOWER | 1439 | 1444 | + | 0 | 1 | TGCAGG |
| FAM069 | SURECOREATSULTR11 | 1449 | 1455 | + | 0 | 1 | GAGACGG |
| FAM302 | SORLIP2AT | 1453 | 1463 | + | 0 | 1 | CGGGCCATCCC |
| FAM002 | SORLIP1AT | 1472 | 1484 | − | 0 | 1 | CAGACGGCCACTC |
| FAM069 | SURECOREATSULTR11 | 1521 | 1527 | − | 0 | 1 | GAGACTA |
| FAM324 | CGCGBOXAT | 1544 | 1549 | + | 0 | 1 | GCGCGC |
| FAM324 | CGCGBOXAT | 1544 | 1549 | − | 0 | 1 | GCGCGC |
| FAM295 | P1BS | 1560 | 1567 | + | 0 | 1 | GCATATGC |
| FAM295 | P1BS | 1560 | 1567 | − | 0 | 1 | GCATATGC |
| FAM098 | CATATGGMSAUR | 1561 | 1566 | + | 0 | 1 | CATATG |
| FAM098 | CATATGGMSAUR | 1561 | 1566 | − | 0 | 1 | CATATG |
| FAM012 | IBOXCORE | 1674 | 1680 | + | 0 | 1 | GATAACA |
| FAM304 | OSE2ROOTNODULE | 1709 | 1713 | + | 0 | 1 | CTCTT |
| FAM209 | RBCSCONSENSUS | 1712 | 1718 | − | 0 | 1 | AATCCAA |
| FAM311 | EECCRCAH1 | 1715 | 1721 | + | 0 | 1 | GATTTAC |
| FAM010 | WBBOXPCWRKY1 | 1753 | 1767 | + | 0 | 1 | TTTGACTTGCAGCCT |
| FAM311 | EECCRCAH1 | 1756 | 1762 | + | 0 | 1 | GACTTGC |
| FAM151 | INTRONLOWER | 1768 | 1773 | + | 0 | 1 | TGCAGG |
| FAM002 | TGACGTVMAMY | 1771 | 1783 | + | 0 | 1 | AGGCATGACGTGG |
| FAM002 | HEXMOTIFTAH3H4 | 1773 | 1785 | − | 0 | 1 | GCCCACGTCATGC |
| FAM002 | ABREOSRAB21 | 1774 | 1786 | + | 0 | 1 | CATGACGTGGGCG |
| FAM002 | UPRMOTIFIAT | 1775 | 1787 | − | 0 | 1 | TCGCCCACGTCAT |
| FAM311 | EECCRCAH1 | 1807 | 1813 | + | 0 | 1 | GAGTTTC |
| FAM272 | SV40COREENHAN | 1813 | 1820 | − | 0 | 1 | GTGGAAAG |
| FAM324 | CGCGBOXAT | 1819 | 1824 | + | 0 | 1 | ACGCGG |
| FAM324 | CGCGBOXAT | 1819 | 1824 | − | 0 | 1 | CCGCGT |
| FAM002 | SORLIP1AT | 1851 | 1863 | + | 0 | 1 | CTGCCCGCCACGT |
| FAM002 | ACGTABREMOTIFA2OSE | 1855 | 1867 | − | 0 | 1 | GAGAACGTGGCGG |
| FAM151 | INTRONLOWER | 1867 | 1872 | − | 0 | 1 | TGCAGG |
| FAM311 | EECCRCAH1 | 1878 | 1884 | − | 0 | 1 | GAGTTGC |
| FAM260 | CAREOSREP1 | 1879 | 1884 | + | 0 | 1 | CAACTC |
| FAM242 | TATABOX3 | 1900 | 1906 | + | 0 | 1 | TATTAAT |
| FAM288 | WUSATAg | 1906 | 1912 | + | 0 | 1 | TTAATGG |

7.) p-KG103

PLACE analysis results of p-KG103 are listed in Table 28. Two TATA Box motifs are found at nucleotide position 879-888 and 880-886 of the forward strand, respectively. No CAAT Box motif is found.

TABLE 28
PLACE analysis results of the 992bp promoter of p-KG103

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM307 | ANAERO3CONSENSUS | 2 | 8 | + | 0 | 1 | TCATCAC |
| FAM065 | AMMORESIIUDCRNIA1 | 7 | 14 | − | 0 | 1 | GGTAGGGT |
| FAM171 | MYBPZM | 8 | 14 | + | 0 | 1 | CCCTACC |
| FAM010 | WBOXNTCHN48 | 19 | 33 | + | 0 | 1 | GCTGACTCGGGCCGC |
| FAM302 | SORLIP2AT | 26 | 36 | + | 0 | 1 | CGGGCCGCAGG |
| FAM263 | DPBFCOREDCDC3 | 45 | 51 | − | 0 | 1 | ACACCGG |
| FAM007 | AUXREPSIAA4 | 49 | 57 | + | 0 | 1 | TGTCCCATC |
| FAM002 | ASF1MOTIFCAMV | 99 | 111 | + | 0 | 1 | CTCTGTGACGACG |
| FAM107 | CGACGOSAMY3 | 107 | 111 | + | 0 | 1 | CGACG |
| FAM147 | HEXAMERATH4 | 107 | 112 | − | 0 | 1 | CCGTCG |
| FAM061 | AGCBOXNPGLB | 111 | 117 | − | 0 | 1 | AGCCGCC |
| FAM304 | OSE2ROOTNODULE | 138 | 142 | − | 0 | 1 | CTCTT |
| FAM002 | HEXMOTIFTAH3H4 | 140 | 152 | + | 0 | 1 | GAGGACGTCAGCA |
| FAM002 | TGACGTVMAMY | 142 | 154 | − | 0 | 1 | CTTGCTGACGTCC |
| FAM057 | ACGTCBOX | 143 | 148 | + | 0 | 1 | GACGTC |
| FAM057 | ACGTCBOX | 143 | 148 | − | 0 | 1 | GACGTC |
| FAM171 | MYBPZM | 157 | 163 | + | 0 | 1 | CCCAACC |
| FAM013 | LTRECOREATCOR15 | 166 | 172 | + | 0 | 1 | TCCGACA |
| FAM013 | LTRECOREATCOR15 | 202 | 208 | + | 0 | 1 | TCCGACG |
| FAM002 | SORLIP1AT | 203 | 215 | + | 0 | 1 | CCGACGGCCACGA |
| FAM107 | CGACGOSAMY3 | 204 | 208 | + | 0 | 1 | CGACG |
| FAM147 | HEXAMERATH4 | 204 | 209 | − | 0 | 1 | CCGTCG |
| FAM002 | SORLIP1AT | 245 | 257 | + | 0 | 1 | CCGGCTGCCACGA |
| FAM107 | CGACGOSAMY3 | 255 | 259 | + | 0 | 1 | CGACG |
| FAM147 | HEXAMERATH4 | 255 | 260 | − | 0 | 1 | CCGTCG |
| FAM089 | BS1EGCCR | 286 | 291 | − | 0 | 1 | AGCGGG |
| FAM002 | ABREMOTIFAOSOSEM | 291 | 303 | − | 0 | 1 | TGCTACGTGTCTA |
| FAM002 | RAV1BAT | 323 | 335 | + | 0 | 1 | GTACACCTGGATC |
| FAM263 | DPBFCOREDCDC3 | 325 | 331 | + | 0 | 1 | ACACCTG |
| FAM205 | PYRIMIDINEBOXOSRAM | 352 | 357 | − | 0 | 1 | CCTTTT |
| FAM302 | SORLIP2AT | 362 | 372 | − | 0 | 1 | AGGGCCCTGGT |
| FAM302 | SORLIP2AT | 365 | 375 | + | 0 | 1 | AGGGCCCTCTC |
| FAM171 | MYBPZM | 382 | 388 | − | 0 | 1 | GCCAACC |
| FAM324 | CGCGBOXAT | 388 | 393 | + | 0 | 1 | CCGCGC |
| FAM324 | CGCGBOXAT | 388 | 393 | − | 0 | 1 | GCGCGG |
| FAM324 | CGCGBOXAT | 390 | 395 | + | 0 | 1 | GCGCGG |
| FAM324 | CGCGBOXAT | 390 | 395 | − | 0 | 1 | CCGCGC |
| FAM069 | ARFAT | 406 | 412 | − | 0 | 1 | CTGTCTC |
| FAM069 | SURECOREATSULTR11 | 406 | 412 | + | 0 | 1 | GAGACAG |
| FAM271 | SEBFCONSSTPR10A | 406 | 412 | − | 0 | 1 | CTGTCTC |
| FAM302 | SORLIP2AT | 423 | 433 | + | 0 | 1 | GGGGCCGCTCG |
| FAM002 | ABREZMRAB28 | 440 | 452 | − | 0 | 1 | GTCCACGTGGGAG |
| FAM085 | BOXCPSAS1 | 440 | 446 | + | 0 | 1 | CTCCCAC |
| FAM002 | ABREZMRAB28 | 441 | 453 | + | 0 | 1 | TCCCACGTGGACG |
| FAM013 | LTRECOREATCOR15 | 479 | 485 | + | 0 | 1 | CCCGACC |
| FAM324 | CGCGBOXAT | 489 | 494 | + | 0 | 1 | GCGCGG |
| FAM324 | CGCGBOXAT | 489 | 494 | − | 0 | 1 | CCGCGC |
| FAM024 | CANBNNAPA | 497 | 505 | + | 0 | 1 | CGAACACGA |
| FAM324 | CGCGBOXAT | 509 | 514 | + | 0 | 1 | CCGCGG |
| FAM324 | CGCGBOXAT | 509 | 514 | − | 0 | 1 | CCGCGG |
| FAM069 | SURECOREATSULTR11 | 535 | 541 | − | 0 | 1 | GAGACCG |
| FAM061 | GCCCORE | 543 | 549 | + | 0 | 1 | GGCCGCC |
| FAM302 | SITEIIATCYTC | 592 | 602 | + | 0 | 1 | TGGGCTGGGGC |
| FAM324 | CGCGBOXAT | 603 | 608 | + | 0 | 1 | ACGCGG |
| FAM324 | CGCGBOXAT | 603 | 608 | − | 0 | 1 | CCGCGT |
| FAM315 | SORLIP5AT | 614 | 620 | − | 0 | 1 | GAGTGAG |
| FAM302 | SITEIIATCYTC | 619 | 629 | − | 0 | 1 | TGGGCCGACGA |
| FAM013 | DRECRTCOREAT | 620 | 626 | − | 0 | 1 | GCCGACG |
| FAM107 | CGACGOSAMY3 | 620 | 624 | − | 0 | 1 | CGACG |
| FAM069 | SURECOREATSULTR11 | 639 | 645 | − | 0 | 1 | GAGACCG |
| FAM013 | DRECRTCOREAT | 651 | 657 | + | 0 | 1 | GCCGACA |
| FAM087 | BOXIINTPATPB | 667 | 672 | + | 0 | 1 | ATAGAA |
| FAM173 | NAPINMOTIFBN | 683 | 689 | + | 0 | 1 | TACACAT |
| FAM172 | MYCATERD | 684 | 690 | − | 0 | 1 | CATGTGT |
| FAM263 | DPBFCOREDCDC3 | 684 | 690 | + | 0 | 1 | ACACATG |
| FAM026 | RYREPEATBNNAPA | 685 | 695 | + | 0 | 1 | CACATGCAATT |
| FAM172 | MYCATRD2 | 685 | 691 | + | 0 | 1 | CACATGC |
| FAM012 | IBOXCORE | 706 | 712 | + | 0 | 1 | GATAATA |
| FAM099 | CCA1ATLHCB1 | 725 | 732 | − | 0 | 1 | AAAAATCT |
| FAM290 | GT1GMSCAM4 | 728 | 733 | − | 0 | 1 | GAAAAA |
| FAM012 | IBOX | 735 | 741 | + | 0 | 1 | GATAAGT |
| FAM266 | MYB1AT | 744 | 749 | + | 0 | 1 | AAACCA |
| FAM003 | REALPHALGLHCB21 | 745 | 755 | + | 0 | 1 | AACCAAATATT |
| FAM002 | SORLIP1AT | 755 | 767 | + | 0 | 1 | TTCACCGCCACAA |
| FAM205 | PYRIMIDINEBOXOSRAM | 766 | 771 | − | 0 | 1 | CCTTTT |
| FAM008 | MYB2AT | 782 | 792 | − | 0 | 1 | TGGGTAACTGA |
| FAM266 | MYB1AT | 800 | 805 | + | 0 | 1 | AAACCA |
| FAM003 | REALPHALGLHCB21 | 801 | 811 | + | 0 | 1 | AACCAAAATAC |
| FAM263 | DPBFCOREDCDC3 | 811 | 817 | − | 0 | 1 | ACACAAG |
| FAM302 | SITEIIATCYTC | 826 | 836 | − | 0 | 1 | TGGGCTCTTGG |
| FAM304 | OSE2ROOTNODULE | 828 | 832 | − | 0 | 1 | CTCTT |
| FAM272 | SV40COREENHAN | 835 | 842 | − | 0 | 1 | GTGGTTTG |
| FAM266 | MYB1AT | 836 | 841 | + | 0 | 1 | AAACCA |
| FAM302 | SITEIIATCYTC | 854 | 864 | − | 0 | 1 | TGGGCTGGGGG |
| FAM240 | TATABOX1 | 879 | 888 | + | 0 | 1 | CTATAAATAC |
| FAM241 | TATABOX2 | 880 | 886 | + | 0 | 1 | TATAAAT |
| FAM002 | SORLIP1AT | 897 | 909 | − | 0 | 1 | ACTTCGGCCACCG |
| FAM315 | SORLIP5AT | 924 | 930 | − | 0 | 1 | GAGTGAG |
| FAM292 | PREATPRODH | 927 | 932 | + | 0 | 1 | ACTCAT |
| FAM306 | ANAERO2CONSENSUS | 955 | 960 | + | 0 | 1 | AGCAGC |
| FAM306 | ANAERO2CONSENSUS | 968 | 973 | + | 0 | 1 | AGCAGC |
| FAM260 | CAREOSREP1 | 983 | 988 | + | 0 | 1 | CAACTC |

8.) p-KG119

PLACE analysis results of p-KG119 are listed in Table 29. Two TATA Box motifs are found at nucleotide position 1925-1931 and 1998-2004 of the forward strand respectively. One CAAT Box motif is found at nucleotide position 214-218 of the forward strand.

TABLE 29
PLACE analysis results of the 2519bp promoter of p-KG119

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM012 | IBOXCORE | 6 | 12 | + | 0 | 1 | GATAAAA |
| FAM151 | INTRONLOWER | 29 | 34 | − | 0 | 1 | TGCAGG |
| FAM227 | SEF1MOTIF | 34 | 42 | + | 0 | 1 | ATATTTATT |
| FAM012 | IBOXCORE | 58 | 64 | + | 0 | 1 | GATAACC |
| FAM325 | MYBCOREATCYCB1 | 63 | 67 | − | 0 | 1 | AACGG |
| FAM262 | CIACADIANLELHC | 93 | 102 | + | 0 | 1 | CAACTAAATC |
| FAM221 | S1FBOXSORPS1L21 | 105 | 110 | − | 0 | 1 | ATGGTA |
| FAM002 | RAV1BAT | 114 | 126 | − | 0 | 1 | TTTCACCTGTCAC |
| FAM271 | SEBFCONSSTPR10A | 114 | 120 | − | 0 | 1 | CTGTCAC |
| FAM322 | BIHD1OS | 115 | 119 | − | 0 | 1 | TGTCA |
| FAM100 | CCAATBOX1 | 126 | 130 | − | 0 | 1 | CCAAT |
| FAM262 | CIACADIANLELHC | 138 | 147 | − | 0 | 1 | CAAGCTGATC |
| FAM170 | AMYBOX1 | 150 | 156 | + | 0 | 1 | TAACAGA |
| FAM303 | OSE1ROOTNODULE | 161 | 167 | + | 0 | 1 | AAAGATA |
| FAM012 | IBOXCORE | 164 | 170 | + | 0 | 1 | GATAAAT |
| FAM266 | MYB1AT | 184 | 189 | + | 0 | 1 | TAACCA |
| FAM276 | TRANSINITDICOTS | 186 | 193 | + | 0 | 1 | ACCATGGC |
| FAM304 | OSE2ROOTNODULE | 209 | 213 | − | 0 | 1 | CTCTT |
| FAM100 | CCAATBOX1 | 214 | 218 | + | 0 | 1 | CCAAT |
| FAM267 | TAAAGSTKST1 | 219 | 225 | + | 0 | 1 | TCTAAAG |
| FAM008 | MYB2AT | 244 | 254 | − | 0 | 1 | TGCCTAACTGC |
| FAM305 | ANAERO1CONSENSUS | 262 | 268 | + | 0 | 1 | AAACAAA |
| FAM008 | MYB2AT | 274 | 284 | + | 0 | 1 | CAGCTAACTGC |
| FAM010 | WBOXATNPR1 | 281 | 295 | − | 0 | 1 | TTTGACACTTAGCAG |
| FAM322 | BIHD1OS | 289 | 293 | + | 0 | 1 | TGTCA |
| FAM030 | -300CORE | 300 | 308 | − | 0 | 1 | TGTAAAGCA |
| FAM267 | TAAAGSTKST1 | 302 | 308 | − | 0 | 1 | TGTAAAG |
| FAM267 | TAAAGSTKST1 | 308 | 314 | + | 0 | 1 | ACTAAAG |
| FAM021 | GT1CORE | 314 | 324 | + | 0 | 1 | GGGTTAAATAT |
| FAM244 | TATABOXOSPAL | 317 | 323 | − | 0 | 1 | TATTTAA |
| FAM306 | ANAERO2CONSENSUS | 359 | 364 | + | 0 | 1 | AGCAGC |
| FAM306 | ANAERO2CONSENSUS | 362 | 367 | + | 0 | 1 | AGCAGC |
| FAM098 | CATATGGMSAUR | 408 | 413 | + | 0 | 1 | CATATG |
| FAM098 | CATATGGMSAUR | 408 | 413 | − | 0 | 1 | CATATG |
| FAM012 | IBOX | 415 | 421 | − | 0 | 1 | GATAAGT |
| FAM014 | SREATMSD | 416 | 422 | + | 0 | 1 | CTTATCC |
| FAM014 | MYBST1 | 417 | 423 | − | 0 | 1 | TGGATAA |
| FAM025 | AMYBOX2 | 418 | 424 | + | 0 | 1 | TATCCAT |
| FAM273 | TATCCAOSAMY | 418 | 424 | + | 0 | 1 | TATCCAT |
| FAM315 | SORLIP5AT | 434 | 440 | − | 0 | 1 | GAGTGAG |
| FAM292 | PREATPRODH | 437 | 442 | + | 0 | 1 | ACTCAT |
| FAM270 | RAV1AAT | 476 | 480 | + | 0 | 1 | CAACA |
| FAM311 | EECCRCAH1 | 520 | 526 | + | 0 | 1 | GAATTCC |
| FAM310 | CPBCSPOR | 530 | 535 | − | 0 | 1 | TATTAG |
| FAM234 | SP8BFIBSP8BIB | 535 | 541 | − | 0 | 1 | TACTATT |
| FAM012 | IBOXCORE | 563 | 569 | + | 0 | 1 | GATAATT |
| FAM234 | SP8BFIBSP8BIB | 595 | 601 | + | 0 | 1 | TACTATT |
| FAM310 | CPBCSPOR | 601 | 606 | + | 0 | 1 | TATTAG |
| FAM014 | MYBST1 | 611 | 617 | − | 0 | 1 | AGGATAT |
| FAM304 | OSE2ROOTNODULE | 660 | 664 | − | 0 | 1 | CTCTT |
| FAM322 | BIHD1OS | 665 | 669 | + | 0 | 1 | TGTCA |
| FAM305 | ANAERO1CONSENSUS | 687 | 693 | + | 0 | 1 | AAACAAA |
| FAM026 | RYREPEATGMGY2 | 699 | 709 | + | 0 | 1 | ATCATGCATAA |
| FAM267 | NTBBF1ARROLB | 760 | 766 | + | 0 | 1 | ACTTTAG |
| FAM267 | TAAAGSTKST1 | 761 | 767 | − | 0 | 1 | TCTAAAG |
| FAM010 | WBOXATNPR1 | 771 | 785 | − | 0 | 1 | TTTGACATTCCACCA |
| FAM272 | SV40COREENHAN | 773 | 780 | + | 0 | 1 | GTGGAATG |
| FAM322 | BIHD1OS | 779 | 783 | + | 0 | 1 | TGTCA |
| FAM267 | NTBBF1ARROLB | 804 | 810 | + | 0 | 1 | ACTTTAT |
| FAM267 | TAAAGSTKST1 | 805 | 811 | − | 0 | 1 | AATAAAG |
| FAM209 | RBCSCONSENSUS | 842 | 848 | + | 0 | 1 | AATCCAA |
| FAM002 | ASF1MOTIFCAMV | 852 | 864 | − | 0 | 1 | TTACCTGACGGGG |
| FAM311 | EECCRCAH1 | 861 | 867 | − | 0 | 1 | GAATTAC |
| FAM270 | RAV1AAT | 869 | 873 | + | 0 | 1 | CAACA |
| FAM013 | LTRECOREATCOR15 | 877 | 883 | − | 0 | 1 | CCCGACA |
| FAM061 | GCCCORE | 890 | 896 | − | 0 | 1 | CGCCGCC |
| FAM171 | MYBPZM | 946 | 952 | + | 0 | 1 | TCCAACC |
| FAM228 | SEF3MOTIFGM | 949 | 954 | + | 0 | 1 | AACCCA |
| FAM171 | MYBPZM | 951 | 957 | + | 0 | 1 | CCCAACC |
| FAM324 | CGCGBOXAT | 971 | 976 | + | 0 | 1 | ACGCGG |
| FAM324 | CGCGBOXAT | 971 | 976 | − | 0 | 1 | CCGCGT |
| FAM190 | OCTAMERMOTIFTAH3H4 | 972 | 979 | + | 0 | 1 | CGCGGATC |
| FAM107 | CGACGOSAMY3 | 984 | 988 | − | 0 | 1 | CGACG |
| FAM245 | TBOXATGAPB | 1029 | 1034 | − | 0 | 1 | ACTTTG |
| FAM270 | RAV1AAT | 1048 | 1052 | − | 0 | 1 | CAACA |
| FAM262 | CIACADIANLELHC | 1052 | 1061 | − | 0 | 1 | CAACATAATC |
| FAM270 | RAV1AAT | 1057 | 1061 | − | 0 | 1 | CAACA |
| FAM273 | TATCCAOSAMY | 1059 | 1065 | − | 0 | 1 | TATCCAA |
| FAM014 | MYBST1 | 1060 | 1066 | + | 0 | 1 | TGGATAA |
| FAM014 | SREATMSD | 1061 | 1067 | − | 0 | 1 | GTTATCC |
| FAM012 | IBOXCORE | 1062 | 1068 | + | 0 | 1 | GATAACC |
| FAM012 | IBOXCORENT | 1097 | 1103 | − | 0 | 1 | GATAAGG |
| FAM087 | BOXIINTPATPB | 1127 | 1132 | − | 0 | 1 | ATAGAA |
| FAM105 | CEREGLUBOX2PSLEGA | 1133 | 1140 | + | 0 | 1 | TGAAAACT |
| FAM292 | PREATPRODH | 1138 | 1143 | + | 0 | 1 | ACTCAT |
| FAM027 | -10PEHVPSBD | 1145 | 1150 | − | 0 | 1 | TATTCT |
| FAM012 | IBOXCORE | 1160 | 1166 | + | 0 | 1 | GATAACA |
| FAM270 | RAV1AAT | 1171 | 1175 | + | 0 | 1 | CAACA |
| FAM100 | CCAATBOX1 | 1209 | 1213 | − | 0 | 1 | CCAAT |
| FAM311 | EECCRCAH1 | 1220 | 1226 | − | 0 | 1 | GACTTCC |
| FAM013 | DRECRTCOREAT | 1223 | 1229 | − | 0 | 1 | GCCGACT |
| FAM013 | LTRECOREATCOR15 | 1244 | 1250 | + | 0 | 1 | CCCGACT |
| FAM311 | EECCRCAH1 | 1280 | 1286 | − | 0 | 1 | GATTTCC |
| FAM325 | MYBCOREATCYCB1 | 1292 | 1296 | + | 0 | 1 | AACGG |
| FAM024 | 2SSEEDPROTBANAPA | 1306 | 1314 | − | 0 | 1 | CAAACACTC |
| FAM310 | CPBCSPOR | 1327 | 1332 | − | 0 | 1 | TATTAG |
| FAM002 | SORLIP1AT | 1337 | 1349 | − | 0 | 1 | ATTTTAGCCACTA |
| FAM069 | ARFAT | 1356 | 1362 | − | 0 | 1 | ATGTCTC |
| FAM069 | SURECOREATSULTR11 | 1356 | 1362 | + | 0 | 1 | GAGACAT |
| FAM024 | PROXBBNNAPA | 1364 | 1372 | + | 0 | 1 | CAAACACCC |
| FAM310 | CPBCSPOR | 1376 | 1381 | − | 0 | 1 | TATTAG |
| FAM300 | LECPLEACS2 | 1379 | 1386 | − | 0 | 1 | TAAAATAT |
| FAM310 | CPBCSPOR | 1433 | 1438 | + | 0 | 1 | TATTAG |
| FAM170 | MYBGAHV | 1453 | 1459 | + | 0 | 1 | TAACAAA |
| FAM281 | MYB1LEPR | 1469 | 1475 | − | 0 | 1 | GTTAGTT |
| FAM024 | 2SSEEDPROTBANAPA | 1481 | 1489 | + | 0 | 1 | CAAACACTG |
| FAM010 | WBOXNTCHN48 | 1518 | 1532 | + | 0 | 1 | TCTGACTGGCCAGCC |
| FAM302 | SITEIIATCYTC | 1524 | 1534 | − | 0 | 1 | TGGGCTGGCCA |
| FAM013 | DRECRTCOREAT | 1559 | 1565 | + | 0 | 1 | GCCGACC |
| FAM061 | GCCCORE | 1568 | 1574 | − | 0 | 1 | GGCCGCC |
| FAM151 | INTRONLOWER | 1573 | 1578 | − | 0 | 1 | TGCAGG |
| FAM012 | IBOXCORE | 1596 | 1602 | − | 0 | 1 | GATAAAA |
| FAM267 | NTBBF1ARROLB | 1618 | 1624 | + | 0 | 1 | ACTTTAT |
| FAM267 | TAAAGSTKST1 | 1619 | 1625 | − | 0 | 1 | TATAAAG |
| FAM010 | WBOXATNPR1 | 1623 | 1637 | − | 0 | 1 | GTTGACAAAGAATAT |
| FAM027 | -10PEHVPSBD | 1624 | 1629 | + | 0 | 1 | TATTCT |
| FAM329 | XYLAT | 1626 | 1633 | − | 0 | 1 | ACAAAGAA |
| FAM322 | BIHD1OS | 1631 | 1635 | + | 0 | 1 | TGTCA |
| FAM003 | REALPHALGLHCB21 | 1635 | 1645 | + | 0 | 1 | AACCAAATACT |
| FAM304 | OSE2ROOTNODULE | 1652 | 1656 | + | 0 | 1 | CTCTT |
| FAM030 | EMHVCHORD | 1684 | 1692 | + | 0 | 1 | TGTAAAGTT |
| FAM202 | -300ELEMENT | 1684 | 1692 | + | 0 | 1 | TGTAAAGTT |
| FAM267 | TAAAGSTKST1 | 1684 | 1690 | + | 0 | 1 | TGTAAAG |
| FAM267 | NTBBF1ARROLB | 1685 | 1691 | − | 0 | 1 | ACTTTAC |
| FAM267 | NTBBF1ARROLB | 1715 | 1721 | + | 0 | 1 | ACTTTAA |
| FAM267 | TAAAGSTKST1 | 1716 | 1722 | − | 0 | 1 | TTTAAAG |
| FAM003 | REALPHALGLHCB21 | 1720 | 1730 | − | 0 | 1 | AACCAACTTTT |
| FAM169 | MYBATRD2 | 1726 | 1732 | − | 0 | 1 | CTAACCA |
| FAM266 | MYB1AT | 1726 | 1731 | − | 0 | 1 | TAACCA |
| FAM013 | DRECRTCOREAT | 1749 | 1755 | + | 0 | 1 | GCCGACT |
| FAM311 | EECCRCAH1 | 1762 | 1768 | − | 0 | 1 | GATTTGC |
| FAM010 | WBOXNTCHN48 | 1809 | 1823 | + | 0 | 1 | TCTGACCGATTTTGA |
| FAM021 | GT1CORE | 1835 | 1845 | + | 0 | 1 | AGGTTAATTCT |
| FAM013 | LTRECOREATCOR15 | 1859 | 1865 | + | 0 | 1 | TCCGACC |
| FAM267 | TAAAGSTKST1 | 1886 | 1892 | + | 0 | 1 | ACTAAAG |
| FAM039 | AACACOREOSGLUB1 | 1897 | 1903 | − | 0 | 1 | AACAAAC |
| FAM305 | ANAERO1CONSENSUS | 1898 | 1904 | − | 0 | 1 | AAACAAA |
| FAM243 | TATABOX4 | 1924 | 1930 | − | 0 | 1 | TATATAA |
| FAM243 | TATABOX4 | 1925 | 1931 | + | 0 | 1 | TATATAA |
| FAM281 | MYB1LEPR | 1946 | 1952 | − | 0 | 1 | GTTAGTT |
| FAM024 | CANBNNAPA | 1948 | 1956 | + | 0 | 1 | CTAACACTT |
| FAM027 | -10PEHVPSBD | 1985 | 1990 | − | 0 | 1 | TATTCT |
| FAM227 | SEF1MOTIF | 1993 | 2001 | + | 0 | 1 | ATATTTATA |
| FAM019 | TATAPVTRNALEU | 1995 | 2007 | + | 0 | 1 | ATTTATATAATTC |
| FAM241 | TATABOX2 | 1995 | 2001 | − | 0 | 1 | TATAAAT |
| FAM243 | TATABOX4 | 1997 | 2003 | − | 0 | 1 | TATATAA |
| FAM243 | TATABOX4 | 1998 | 2004 | + | 0 | 1 | TATATAA |
| FAM305 | ANAERO1CONSENSUS | 2009 | 2015 | + | 0 | 1 | AAACAAA |
| FAM310 | CPBCSPOR | 2021 | 2026 | − | 0 | 1 | TATTAG |
| FAM266 | MYB1AT | 2034 | 2039 | − | 0 | 1 | AAACCA |
| FAM270 | RAV1AAT | 2057 | 2061 | − | 0 | 1 | CAACA |
| FAM324 | CGCGBOXAT | 2071 | 2076 | + | 0 | 1 | GCGCGC |
| FAM324 | CGCGBOXAT | 2071 | 2076 | − | 0 | 1 | GCGCGC |
| FAM270 | RAV1AAT | 2077 | 2081 | + | 0 | 1 | CAACA |
| FAM013 | DRE2COREZMRAB17 | 2112 | 2118 | + | 0 | 1 | ACCGACT |
| FAM234 | SP8BFIBSP8BIB | 2120 | 2126 | + | 0 | 1 | TACTATT |
| FAM300 | LECPLEACS2 | 2129 | 2136 | + | 0 | 1 | TAAAATAT |
| FAM124 | ERELEE4 | 2137 | 2144 | + | 0 | 1 | AATTCAAA |
| FAM061 | GCCCORE | 2189 | 2195 | − | 0 | 1 | CGCCGCC |
| FAM107 | CGACGOSAMY3 | 2194 | 2198 | − | 0 | 1 | CGACG |
| FAM010 | WBOXATNPR1 | 2211 | 2225 | − | 0 | 1 | GTTGACGCATGGTGC |
| FAM002 | ASF1MOTIFCAMV | 2216 | 2228 | − | 0 | 1 | AGCGTTGACGCAT |
| FAM324 | CGCGBOXAT | 2244 | 2249 | + | 0 | 1 | GCGCGC |
| FAM324 | CGCGBOXAT | 2244 | 2249 | − | 0 | 1 | GCGCGC |
| FAM270 | RAV1AAT | 2250 | 2254 | − | 0 | 1 | CAACA |
| FAM324 | CGCGBOXAT | 2269 | 2274 | + | 0 | 1 | ACGCGT |
| FAM324 | CGCGBOXAT | 2269 | 2274 | − | 0 | 1 | ACGCGT |
| FAM002 | SORLIP1AT | 2271 | 2283 | − | 0 | 1 | CCATTTGCCACGC |
| FAM026 | RYREPEATGMGY2 | 2282 | 2292 | + | 0 | 1 | GGCATGCATTC |
| FAM013 | LTRECOREATCOR15 | 2320 | 2326 | + | 0 | 1 | CCCGACG |
| FAM107 | CGACGOSAMY3 | 2322 | 2326 | + | 0 | 1 | CGACG |
| FAM324 | CGCGBOXAT | 2324 | 2329 | + | 0 | 1 | ACGCGG |
| FAM324 | CGCGBOXAT | 2324 | 2329 | − | 0 | 1 | CCGCGT |
| FAM002 | LRENPCABE | 2334 | 2346 | + | 0 | 1 | CAGGACGTGGCAG |
| FAM002 | SORLIP1AT | 2338 | 2350 | − | 0 | 1 | CGCTCTGCCACGT |
| FAM061 | GCCCORE | 2349 | 2355 | + | 0 | 1 | CGCCGCC |
| FAM267 | TAAAGSTKST1 | 2393 | 2399 | − | 0 | 1 | AATAAAG |
| FAM194 | PALBOXAPC | 2400 | 2406 | + | 0 | 1 | CCGTCCT |
| FAM010 | WBOXHVISO1 | 2404 | 2418 | − | 0 | 1 | TGTGACTGAGCAAGG |
| FAM315 | SORLIP5AT | 2430 | 2436 | − | 0 | 1 | GAGTGAG |
| FAM194 | PALBOXAPC | 2440 | 2446 | + | 0 | 1 | CCGTCCG |
| FAM069 | SURECOREATSULTR11 | 2459 | 2465 | − | 0 | 1 | GAGACGA |
| FAM085 | BOXCPSAS1 | 2463 | 2469 | + | 0 | 1 | CTCCCAC |
| FAM013 | LTRECOREATCOR15 | 2500 | 2506 | + | 0 | 1 | CCCGACG |
| FAM107 | CGACGOSAMY3 | 2502 | 2506 | + | 0 | 1 | CGACG |
| FAM324 | CGCGBOXAT | 2504 | 2509 | + | 0 | 1 | ACGCGC |
| FAM324 | CGCGBOXAT | 2504 | 2509 | − | 0 | 1 | GCGCGT |

9.) p-KG129

PLACE analysis results of p-KG129 are listed in Table 30. No TATA Box motifs are found in this promoter. One CAAT Box motif is found at nucleotide position 244-248 of the forward strand.

TABLE 30
PLACE analysis results of the 512 bp promoter of p-KG129

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM263 | DPBFCOREDCDC3 | 20 | 26 | + | 0 | 1 | ACACTAG |
| FAM089 | BS1EGCCR | 25 | 30 | + | 0 | 1 | AGCGGG |
| FAM306 | ANAERO2CONSENSUS | 30 | 35 | − | 0 | 1 | AGCAGC |
| FAM267 | TAAAGSTKST1 | 33 | 39 | + | 0 | 1 | GCTAAAG |
| FAM267 | TAAAGSTKST1 | 52 | 58 | + | 0 | 1 | GCTAAAG |
| FAM303 | OSE1ROOTNODULE | 55 | 61 | + | 0 | 1 | AAAGATA |
| FAM263 | DPBFCOREDCDC3 | 65 | 71 | + | 0 | 1 | ACACTAG |
| FAM003 | MYBPLANT | 70 | 80 | − | 0 | 1 | CACCAACCGCT |
| FAM171 | BOXLCOREDCPAL | 73 | 79 | − | 0 | 1 | ACCAACC |
| FAM290 | GT1GMSCAM4 | 94 | 99 | + | 0 | 1 | GAAAAA |
| FAM002 | HEXMOTIFTAH3H4 | 140 | 152 | + | 0 | 1 | AAAAACGTCAGTG |
| FAM002 | TGACGTVMAMY | 142 | 154 | − | 0 | 1 | TTCACTGACGTTT |
| FAM002 | ASF1MOTIFCAMV | 166 | 178 | − | 0 | 1 | TATAGTGACGATC |
| FAM087 | BOXIINTPATPB | 183 | 188 | − | 0 | 1 | ATAGAA |
| FAM272 | SV40COREENHAN | 196 | 203 | + | 0 | 1 | GTGGTTAG |
| FAM169 | MYBATRD2 | 197 | 203 | − | 0 | 1 | CTAACCA |
| FAM266 | MYB1AT | 197 | 202 | − | 0 | 1 | TAACCA |
| FAM266 | MYB1AT | 241 | 246 | + | 0 | 1 | AAACCA |
| FAM003 | REALPHALGLHCB21 | 242 | 252 | + | 0 | 1 | AACCAATACTA |
| FAM100 | CCAATBOX1 | 244 | 248 | + | 0 | 1 | CCAAT |
| FAM087 | BOXIINTPATPB | 289 | 294 | − | 0 | 1 | ATAGAA |
| FAM002 | ASF1MOTIFCAMV | 334 | 346 | − | 0 | 1 | TTCTGTGACGACG |
| FAM107 | CGACGOSAMY3 | 334 | 338 | − | 0 | 1 | CGACG |
| FAM061 | GCCCORE | 370 | 376 | − | 0 | 1 | GGCCGCC |
| FAM002 | SORLIP1AT | 372 | 384 | + | 0 | 1 | CGGCCGGCCACGT |
| FAM002 | ABREATCONSENSUS | 376 | 388 | − | 0 | 1 | GGGTACGTGGCCG |
| FAM324 | CGCGBOXAT | 394 | 399 | + | 0 | 1 | ACGCGT |
| FAM324 | CGCGBOXAT | 394 | 399 | − | 0 | 1 | ACGCGT |
| FAM107 | CGACGOSAMY3 | 397 | 401 | − | 0 | 1 | CGACG |
| FAM002 | GADOWNAT | 408 | 420 | − | 0 | 1 | CAACACGTGTCCT |
| FAM002 | CACGTGMOTIF | 409 | 421 | + | 0 | 1 | GGACACGTGTTGG |
| FAM263 | DPBFCOREDCDC3 | 411 | 417 | + | 0 | 1 | ACACGTG |
| FAM263 | DPBFCOREDCDC3 | 412 | 418 | − | 0 | 1 | ACACGTG |
| FAM024 | CANBNNAPA | 413 | 421 | − | 0 | 1 | CCAACACGT |
| FAM270 | RAV1AAT | 416 | 420 | − | 0 | 1 | CAACA |
| FAM010 | WBOXNTCHN48 | 421 | 435 | + | 0 | 1 | GCTGACCGGACAGTT |
| FAM087 | BOXIINTPATPB | 465 | 470 | + | 0 | 1 | ATAGAA |
| FAM107 | CGACGOSAMY3 | 479 | 483 | + | 0 | 1 | CGACG |
| FAM107 | CGACGOSAMY3 | 482 | 486 | + | 0 | 1 | CGACG |
| FAM147 | HEXAMERATH4 | 482 | 487 | − | 0 | 1 | CCGTCG |
| FAM061 | GCCCORE | 486 | 492 | − | 0 | 1 | CGCCGCC |
| FAM061 | GCCCORE | 489 | 495 | − | 0 | 1 | TGCCGCC |

### Example 6

### Binary Vector Construction for Maize Transformation to Evaluate the Function of the Promoters

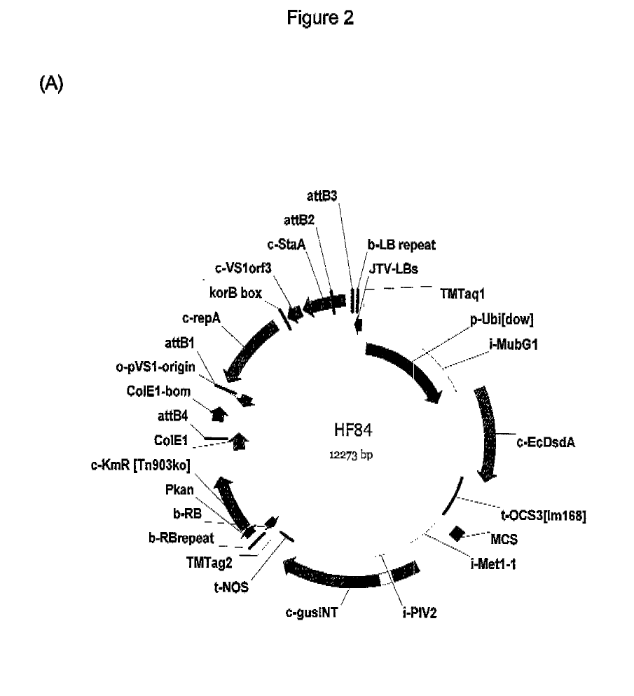

To facilitate subcloning, the promoter fragments of KG24, 37, 45, 46, 49, 103, 119, 129 were modified by the addition of a Pad restriction enzyme site (for p_KG24, p_KG37, p_KG45, p_KG46, p_KG49, p_KG103, p_KG119, p_KG129) or a NotI (for p_KG56) at its 5′ end and a NotI site (for p_KG24, p_KG103, p_KG129) or a BsiWI site (for p_KG37, p_KG45, p_KG46, p_KG49, p_KG56,) at its 3′ end. The PacI-pKG37 (or 45, 46, 49)-Bs/WI, or PacI-pK24 (or 103, 119)-NotI or NotI-pKG56-BsANI promoter fragment was digested and ligated into a corresponding enzyme digested BPS basic binary vector HF84. HF84 comprises a plant selectable marker expression cassette (p-Ubi::c-EcEsdA::t-OCS3), as well as a promoter evaluation cassette that consists of a multiple cloning site (MCS) for insertion of promoter and the rice MET1-1 intron to supply intron-mediated enhancement in monocot cells, GUS reporter gene, and NOS terminator. Diagram of HF84 is shown in FIG. 2 A. p-KG129 fragment was cloned into a binary vector backbone RCB1006 (FIG. 2 B) via Gateway reaction.

Table 31 lists the resulting binary vector of the KG promoters, Sequences of the promoter cassettes in the binary vectors are shown in SEQ ID NO: 57, 58, and 62-68.

TABLE 31
Binary vectors of the KG promoters for corn transformation

| Promoter | Vector | | SEQ ID |
|---|---|---|---|
| ID | ID | Description | OF VECTOR |
| p-KG24 | RHF155 | p-KG24::iMET1::GUS::t-NOS | 63 |
| p-KG37 | RKF109 | p-KG37::iMET1::GUS::t-NOS | 64 |
| p-KG45 | RKF106 | p-KG45::iMET1::GUS::t-NOS | 65 |
| p-KG46 | RKF107 | p-KG46::iMET1::GUS::t-NOS | 66 |
| p-KG49 | RKF108 | p-KG49::iMET1::GUS::t-NOS | 62 |
| p-KG56 | RKF125 | p-KG56::iMET1::GUS::t-NOS | 57 |
| p-KG103 | RHF128 | p-KG103::iMET1::GUS::t-NOS | 67 |
| p-KG119 | RHF138 | p-KG119::iMET1::GUS::t-NOS | 68 |
| p-KG129 | RTP1047 | p-KG129::iMET1::GUS::t-NOS | 58 |

### Example 7

### Promoter Evaluation in Transgenic Maize with the KG Promoters

Expression patterns and levels driven by the KG promoters were measured using GUS histochemical analysis following the protocol in the art (Jefferson 1987). Maize transformation was conducted using an Agrobacterium-mediated transformation system. Ten and five single copy events for T0 and T1 plants were chosen for the promoter analysis. GUS expression was measured at various developmental stages:

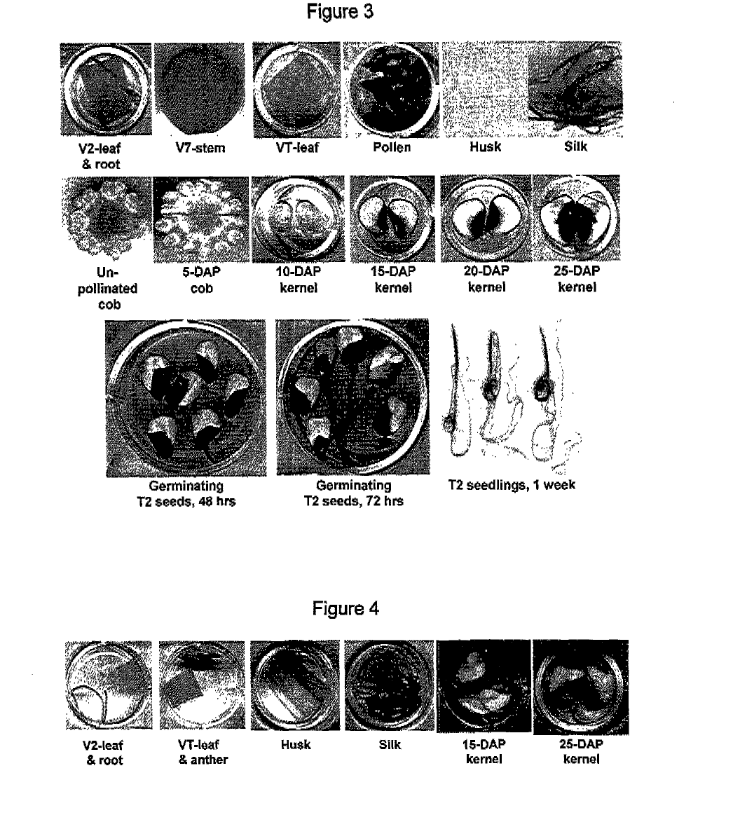

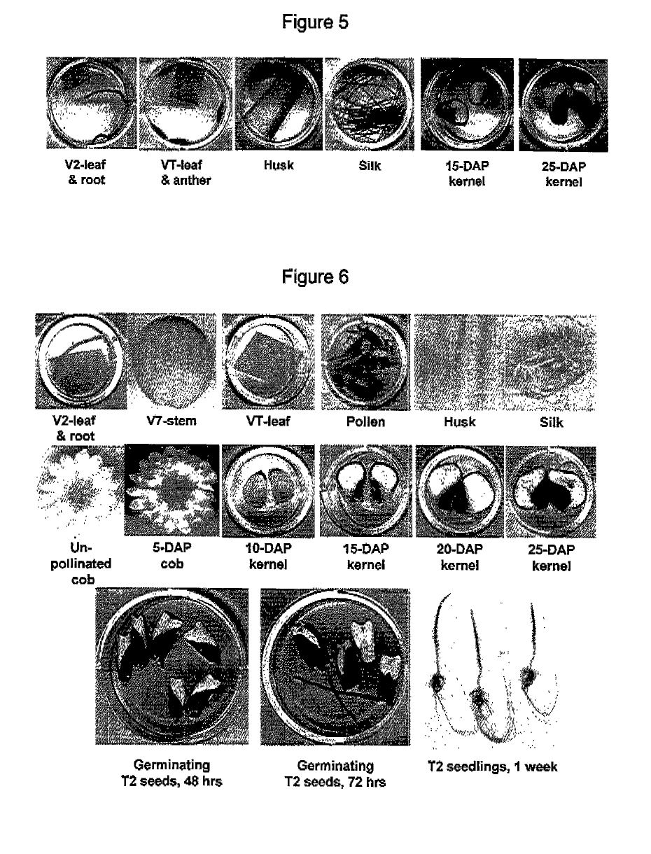

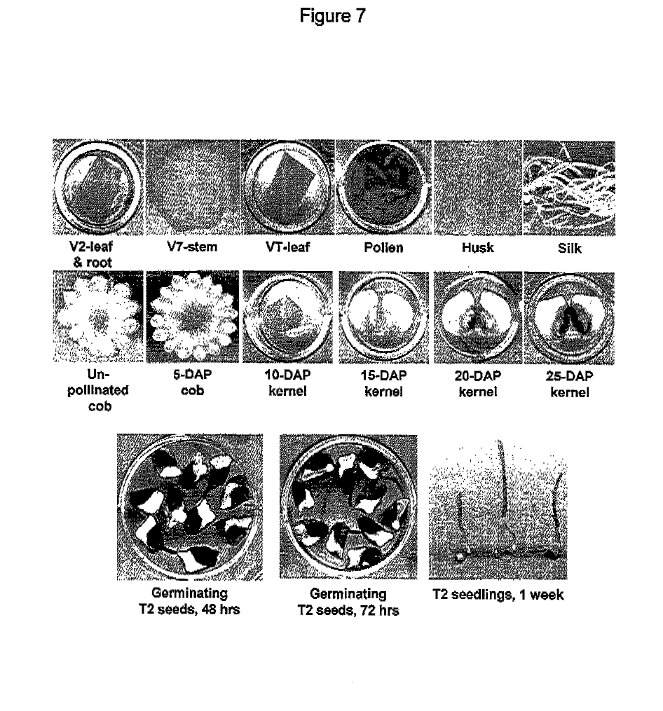

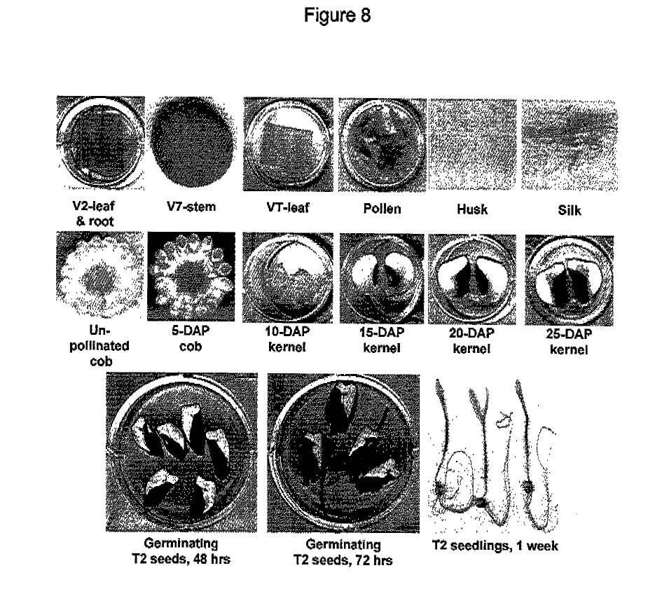

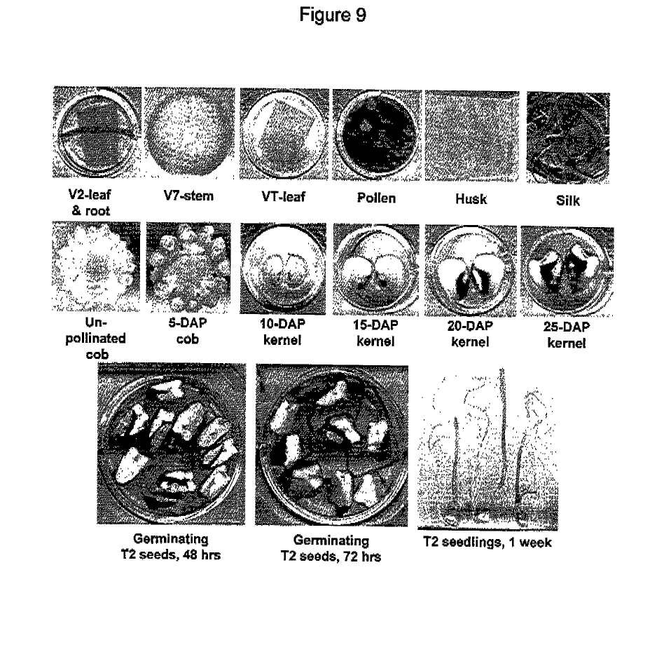

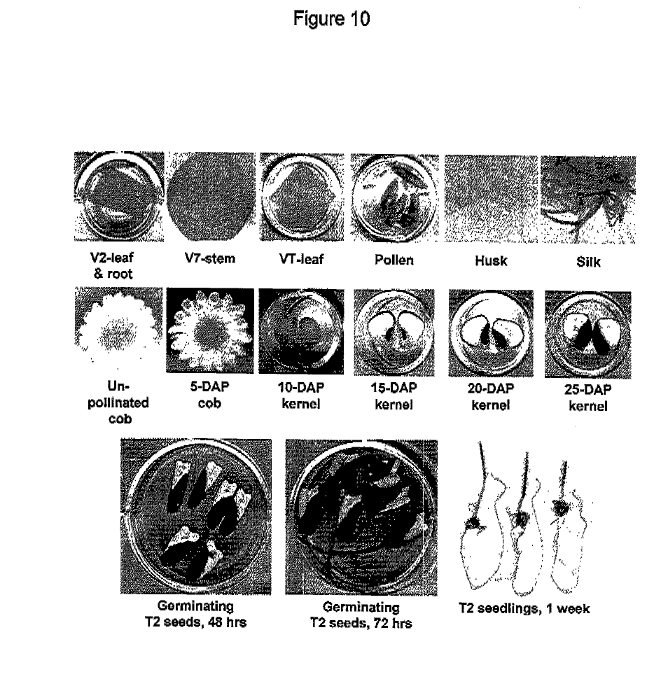

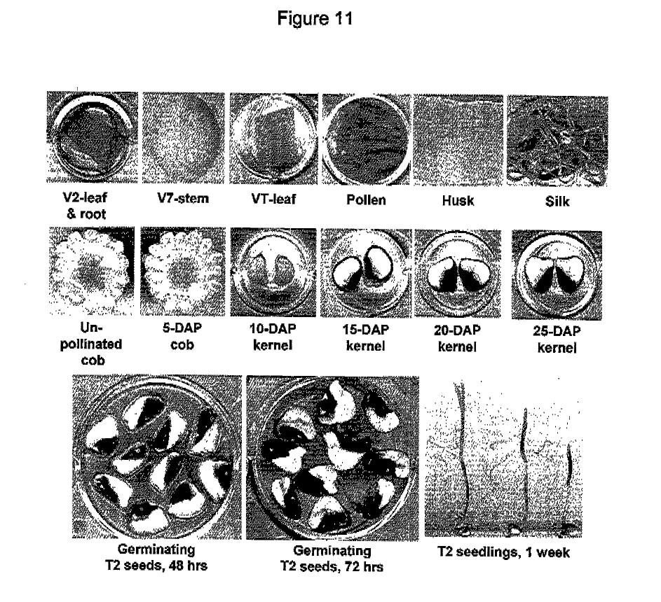

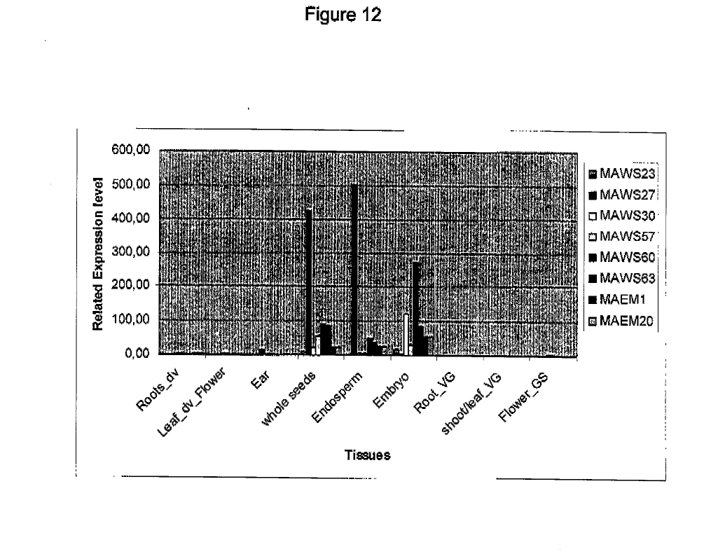

1) Roots and leaves at 5-leaf stage
2) Stem at V-7 stage
2) Leaves, husk and silk at flowering stage (first emergence of silk)
3) Spikelets/Tassel (at pollination)
5) Ear or Kernels at 5, 10, 15, 20, and 25 days after pollination (DAP)
The results indicated that all these 9 promoters expressed specifically in pollen and in embryo (FIGS. 4 to 11).

### Example 8

### Identification of MA-Transcript Candidates

A microarray study was conducted to identify transcripts with whole seed-specific and or embryo-specific expression in maize using a battery of RNA samples from 23 maize tissues generated by BASF (Table 32). The twenty-three labeled RNAs of these maize tissues were hybridized separately to 23 of our custom designed BPS maize Affymetrix chips, labeled with fluorescent streptavidin antibody, washed, stained and scanned as instructed in the Affymetrix Expression Analysis Technical Manual.

TABLE 32
Corn Tissues used for mRNA expression profiling experiment

| Sample | | Timing and | Days after |
|---|---|---|---|
| No. | Tissue | number of plants | Pollination |
| 1 | Root | 9 am (4 plants) | 5 |
| 2 | | 9 am (4 plants) | 15 |
| 3 | | 9 am (4 plants) | 30 |
| 4 | leaf above the ear | 9 am (6 plants) | 5 |
| 5 | | 9 am (6 plants) | 15 |
| 6 | | 9 am (6 plants) | 30 |
| 7 | ear complete | 9 am (6 plants) | 5 |
| 8 | | 9 am (6 plants) | 10 |
| 9 | Whole seed | 9 am (6 plants) | 15 |
| 10 | | 9 am (6 plants) | 20 |
| 11 | | 9 am (6 plants) | 30 |
| 12 | Endosperm | 9 am (6 plants) | 15 |
| 13 | | 9 am (6 plants) | 20 |
| 14 | | 9 am (6 plants) | 30 |
| 15 | Embryo | 9 am (6 plants) | 15 |
| 16 | | 9 am (6 plants) | 20 |
| 17 | | 9 am (6 plants) | 30 |
| 18 | Female pistilate flower | 6 plants | before |
| | | | pollination |
| 19 | germinating seed | 20 seeds | imbibition |
| | | | for 3 days |
| 20 | root, veg. state | | V2 |
| 21 | root, veg. state | | V4 |
| 22 | leaf, veg. State | | V2 |
| 23 | leaf, veg. State | | V4 |

The chip hybridization data were analyzed using Genedata Specialist software and relative expression level was determined based on the hybridization signal intensity of each tissue. Eight of the BPS maize chip probe sets were selected as candidate transcripts showing 3-8 fold higher expression in whole seeds and or in embryo as compared to other tissues. Corresponding transcripts of these probe sets were named as MAWS23, MAWS27, MAWS30, MAWS57, MAWS60, MAWS63, MAEM1 and MAEM20 (Table 32-1). Consensus sequences of the selected chip probe sets are shown in SEQ ID NOs 91, 92, 95-97, 105-107.

TABLE 32-1
Microarray candidates and probe sets

| MA Candidates | Proble set | SEQ ID |
|---|---|---|
| MAWS23 | ZM1s57912912 | 105 |
| MAWS27 | ZM3s00207 | 96 |
| MAWS30 | ZM1a61269071 | 106 |
| MAWS57 | ZM1s57500283 | 107 |
| MAWS60 | ZM4s20063 | 91 |
| MAWS63 | ZM1s62013293 | 97 |
| MAEM1 | ZM4s09689 | 92 |
| MAEM20 | ZM1s5153555 | 95 |

### Example 9

### Confirmation of Expression Pattern of the MA Candidates Using Quantitative Reverse Transcriptase-Polymerase Chain Reaction (Q-RT-PCR)

In order to confirm the native expression pattern of the MA candidates, quantitative reverse transcription PCR (q-RT-PCR) was performed using total RNA isolated from the same materials as were used for the chip hybridization (Table 32).

Primers for qRT-PCR were designed based on the consensus sequences of probe sets shown in Table 2 using the Vector NTI software package (Invitrogen, Carlsbad, Calif., USA). Two sets of primers were used for PCR amplification for each candidate. The glyceraldehyde-3-phosphate dehydrogenase (GAPDH) gene served as a control for normalization purposes. Sequences of primers for q-RT-PCR are listed in Table 32-2.

TABLE 32-2
Primer sequences for q-RT-PCR

| Primer | Sequences |
|---|---|
| MAWS23_forward_1 | TCCTCCTCGATCCATCGATC |
| MAWS23_reverse_1 | TTCACCTGCTCACCCATCGG |
| MAWS23_forward_2 | GGCTTCCTCGTAAGCAAGTCATCCA |
| MAWS23_reverse_2 | AACACAGCATTCCGCGACGACC |
| MAWS27_forward_1 | CCGTCCACCGTGAACTCCGCGT |
| MAWS27_reverse_1 | TGGCAGCATCCTGACGCTAACCAG |
| MAWS27_forward_2 | CGTCAGGATGCTGCCATGGGC |
| MAWS27_reverse_2 | TCCGGCGCGTTCTCGTACGA |
| MAWS30_forward_1 | GATGGGTGAGCAGGTGAAGG |
| MAWS30_reverse_1 | AAGAGCAGGAACACGGGCGT |
| MAWS30_forward_2 | ATCCAGAGCAAGGCGCAGGA |
| MAWS30_reverse_2 | TTGACACGCACGCATCCATG |
| MAWS57_forward_1 | CGCCCAACTCGACGCAGGTG |
| MAWS57_reverse_1 | CTGGTGAGCAGCGCGATGGG |
| MAWS57_forward_2 | CTCCCCGTGGCCACCTGGATGT |
| MAWS57_reverse_2 | CGCAGGTATCCGCCGTACTCGC |
| MAWS60_forward_1 | CGACGGACGGGTCCAGACAGCA |
| MAWS60_reverse_1 | TGCACGCGAGCCACCAGGAC |
| MAWS60_forward_2 | AGGGCTCCACGCTCCTTACCGAA |
| MAWS60_reverse_2 | GTTCCCGGCGCCATCCCTATC |
| MAWS63_forward_1 | CAAGCGCGAAATCAAGCCCGG |
| MAWS63_reverse_1 | GGCAGCGGCGAAGAGGTCGA |
| MAWS63_forward_2 | GGGGACCAACAAGAACGCCGTC |
| MAWS63_reverse_2 | TCCCAAGCGACGTCCACCGG |
| MAEM1_forward_1 | CTGGTGGTGGGGCGGGTGAT |
| MAEM1_reverse_1 | GGGGTCCGTCATGATCAGCG |
| MAEM1_forward_2 | GACCATGAGAGAGTACCTCCAC |
| MAEM1_reverse_2 | GAACAGCACCAGCACGTAGC |
| MAEM20_forward_1 | TGCCACTGTGCTGTGCAGTA |
| MAEM20_reverse_1 | GAGCCCACCACCTTGTTTCC |
| MAEM20_forward_2 | TCCACGGTGGTGCATGTCGT |
| MAEM20_reverse_2 | TACTGCTGCAGAATCCTCCTCCGG |
| GAPDH_Forward | GTAAAGTTCTTCCTGATCTGAAT |
| GAPDH_Reverse | TCGGAAGCAGCCTTAATA |

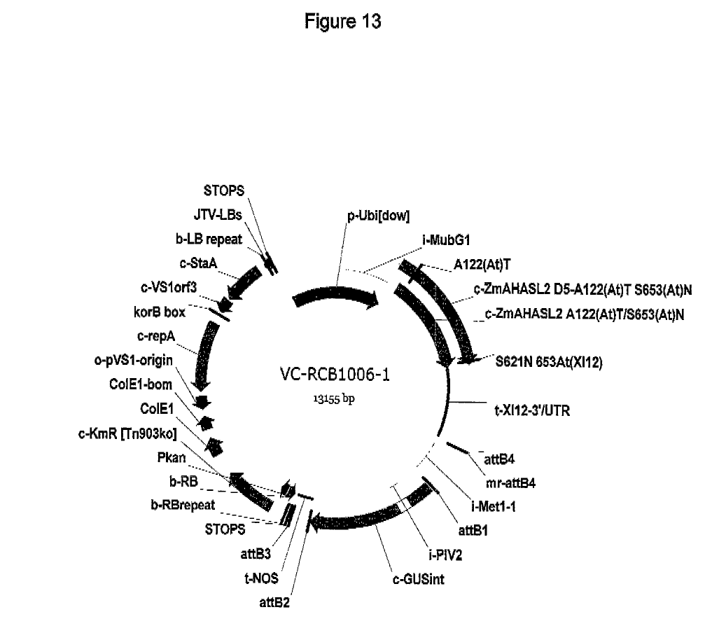

q-RT-PCR was performed using SuperScript III Reverse Transcriptase (Invitrogen, Carlsbad, Calif., USA) and SYBR Green QPCR Master Mix (Eurogentec, San Diego, Calif., USA) in an ABI Prism 7000 sequence detection system. In brief, cDNA was synthesized using 2-3 μg of total RNA and 1 μL reverse transcriptase in a 20 μL volume. The cDNA was diluted to a range of concentrations (15-20 ng/μL). Thirty to forty ng of cDNA was used for quantitative PCR (qPCR) in a 30 μL volume with SYBR Green QPCR Master Mix following the manufacturer's instruction. The thermocycling conditions were as follows: incubate at 50° C. for 2 minutes, denature at 95° C. for 10 minutes, and run 40 cycles at 95° C. for 15 seconds and 60° C. for 1 minute for amplification. After the final cycle of the amplification, the dissociation curve analysis was carried out to verify that the amplification occurred specifically and no primer dimer product was generated during the amplification process. The housekeeping gene glyceraldehyde-3-phosphate-dehydrogenase (GAPDH, primer sequences in Table 3) was used as an endogenous reference gene to normalize the calculation using the Comparative Ct (Cycle of threshold) value method. The ΔCT value was obtained by subtracting the Ct value of GAPDH gene from the Ct value of the candidate gene, and the relative transcription quantity (expression level) of the candidate gene expression was presented as 2-ΔCT. The q-RT-PCR results are summarized in FIG. 12. All candidates showed similar expression patterns that are equivalent to the expression patterns obtained from the chip hybridization study.

### Example 10

### Annotation and Promoter Identification of the MA Candidates

The coding sequences of the MA candidates were annotated based on in silico results obtained from both BLASTX of each EST sequence against GenBank protein database (nr) and the results of in silico translation of the sequence using Vector NTI software package.

### 1. Annotation of MAWS23

MAWS23 encodes Lipid body-associated protein L2 (Maize Oleosin 18 kDa) (GenBank Accession: P21641). The top 10 homologous sequences identified in the BlastX query are presented in Table 33.

TABLE 33
BLASTX search results of the maize ZM1s57912912 (MAWS23)

| | | | | % Iden- |
|---|---|---|---|---|
| Accession | Description | Score | E-value | tities |
| P21641 | OLEO3_MAIZE | 69 | 4.00E−34 | 100 |
| | Oleosin Zm-II | | | |
| | (Oleosin 18 kDa) | | | |
| | (Lipid body- | | | |
| | associated protein L2) | | | |
| AAA68066.1 | 17 kDa oleosin | 65 | 5.00E−29 | 93 |
| NP_001050984.1 | Os03g0699000 [Oryza | 63 | 8.00E−20 | 93 |
| | sativa (japonica | | | |
| | cultivar-group)] | | | |
| CAA57994.1 | high molecular weight | 60 | 4.00E−19 | 86 |
| | oleosin [Hordeum | | | |
| | vulgare subsp. vulgare] | | | |
| AAC02240.1 | 18 kDa oleosin | 60 | 5.00E−19 | 90 |
| | [Oryza sativa] | | | |
| CAN80217.1 | hypothetical protein | 70 | 1.00E−09 | 54 |
| | [Vitis vinifera] | | | |
| AAB24078.1 | lipid body membrane | 59 | 9.00E−07 | 83 |
| | protein [Daucus carota] | | | |
| AAG43516.1 | AF210696_1 15 kD | 52 | 2.00E−06 | 66 |
| | oleosin-like protein 1 | | | |
| | [Perilla frutescens] | | | |
| AAG43517.1 | AF210697_1 15 kD | 52 | 2.00E−06 | 66 |
| | oleosin-like protein 2 | | | |
| | [Perilla frutescens] | | | |
| CAN80218.1 | hypothetical protein | 59 | 2.00E−06 | 80 |
| | [Vitis vinifera] | | | |

The CDS sequence of the gene corresponding to MAWS23 is shown in SEQ ID NO: 33 and the translated amino acid sequence is shown in SEQ ID NO: 51.

### Identification of the Promoter Region of MAWS23

For our promoter identification purposes, the sequence upstream of the start codon of the MAWS23 gene was defined as the promoter p-MAWS23. To identify this predicted promoter region, the sequence of ZM1s57912912 was mapped to the BASF Plant Science proprietary maize genomic DNA sequence database, PUB_tigr_maize_genomic_partial_{—}5.0.nt. One maize genomic DNA sequences, AZM5_{—}84556 (2036 bp, SEQ ID NO 87) was identified. This 2036 bp sequence harbored the predicted CDS of the corresponding gene to MAWS23 and less than 0.5 kb upstream sequence of the ATG start codon of this gene. In addition, a public available sequence CL990349 was overlapped with AZM5_{—}84556. The contig of these 2 genomic sequences containing 1.3 kb upstream region is shown in SEQ ID NO: 87.

### Isolation of the Promoter Region by PCR Amplification

The putative promoter region was isolated via genomic PCR using the following sequence specific primers:

Forward primer:

(SEQ ID NO: 172)

TCATCGGTACTCGCGATGTC
Reverse primer:

(SEQ ID NO: 173)

CTTTGCAAACAAAGTGACGGAG.

The expected 1264 bp fragment was amplified from maize genomic DNA, and named as promoter MAWS23 (p-MAWS23). Sequence of p-MAWS23 is shown in SEQ ID NO: 15.
BLASTN Results of p_MAWS23

The top 20 homologous sequences identified in the BlastN query of p_MAWS23 are presented in Table 34.

TABLE 34
BLASTN results of p_MAWS23

| | | Max | Total | Query | | Max |
|---|---|---|---|---|---|---|
| Accession | Description | score | score | coverage | E value | ident |
| J05212.1 | Maize oleosin KD18 (KD18; L2) | 2150 | 2150 | 100% | 0 | 97% |
| | gene, complete cds | | | | | |
| AY555143.1 | Zea may BAC clone c573L14, | 682 | 682 | 37% | 0 | 91% |
| | complete sequence | | | | | |
| AF488416.1 | Zea mays chromosome 9 BAC 9C20 | 641 | 1094 | 37% | 2.00E−180 | 88% |
| | complete sequence | | | | | |
| AF434192.1 | Zea mays line LH82 transposon Ins2, | 625 | 625 | 37% | 2.00E−175 | 88% |
| | YZ1 (yz1) gene, YZ1-LH82 allele, | | | | | |
| | complete cds; tRNA-Phe (trnF) gene, | | | | | |
| | complete sequence; retrotransposon | | | | | |
| | Machiavelli Gag and Pol (gag/pol) | | | | | |
| | gene, complete cds; and | | | | | |
| | retrotransposon-like Ozymandias | | | | | |
| | and MITE Gnat1, complete sequence | | | | | |
| AY455286.1 | Zea mays chloroplast phytoene | 619 | 696 | 37% | 8.00E−174 | 87% |
| | synthase (Y1) gene, complete cds; | | | | | |
| | nuclear gene for chloroplast product | | | | | |
| AF090447.2 | Zea mays 22 kDa alpha zein gene | 571 | 680 | 38% | 4.00E−159 | 86% |
| | cluster, complete sequence | | | | | |
| AC157977.1 | Genomic sequence for Zea mays | 556 | 556 | 37% | 8.00E−155 | 84% |
| | chromosome 8 BAC clone | | | | | |
| | ZMMBBb0284N04, complete | | | | | |
| | sequence | | | | | |
| BT038288.1 | Zea mays full-length cDNA clone | 522 | 522 | 36% | 2.00E−144 | 83% |
| | ZM_BFb0224G21 mRNA, complete | | | | | |
| | cds | | | | | |
| L29505.1 | Zea mays high sulfur zein gene, | 520 | 520 | 29% | 6.00E−144 | 90% |
| | complete cds | | | | | |
| EU943322.1 | Zea mays clone 1599166 mRNA | 477 | 477 | 37% | 6.00E−131 | 80% |
| | sequence | | | | | |
| AC165176.2 | Zea mays clone ZMMBBb-177G21, | 475 | 635 | 37% | 2.00E−130 | 90% |
| | complete sequence | | | | | |
| AC165171.2 | Zea mays clone CH201-145P10, | 466 | 466 | 36% | 1.00E−127 | 81% |
| | complete sequence | | | | | |
| AC152494.1 | Zea mays BAC clone Z418K17, | 464 | 948 | 37% | 4.00E−127 | 80% |
| | complete sequence | | | | | |
| X73151.1 | Z. mays GapC2 gene | 461 | 461 | 38% | 5.00E−126 | 79% |
| AC165267.2 | Zea mays clone ZMMBBb-151F20, | 446 | 446 | 37% | 1.00E−121 | 79% |
| | complete sequence | | | | | |
| DQ002407.1 | Zea mays copia retrotransposon | 428 | 548 | 37% | 3.00E−116 | 82% |
| | opie1, gypsy retrotransposon | | | | | |
| | grande1, xilon1 retrotransposon, | | | | | |
| | helitron B73_14578, gypsy | | | | | |
| | retrotransposon huck1 and ruda | | | | | |
| | retrotransposon, complete sequence | | | | | |
| AF546189.1 | Contiguous genomic DNA sequence | 340 | 386 | 24% | 1.00E−89 | 84% |
| | comprising the 19-kDa-zein gene | | | | | |
| | family from Zea mays, complete | | | | | |
| | sequence | | | | | |
| AY109359.1 | Zea mays CL2022_2 mRNA | 306 | 306 | 27% | 2.00E−79 | 77% |
| | sequence | | | | | |
| BT038370.1 | Zea mays full-length cDNA clone | 288 | 288 | 21% | 6.00E−74 | 83% |
| | ZM_BFb0229A02 mRNA, complete | | | | | |
| | cds | | | | | |
| EU953088.1 | Zea mays clone 1381669 unknown | 201 | 201 | 20% | 7.00E−48 | 75% |
| | mRNA | | | | | |

### 2. Annotation of MAWS27

MAWS27 encodes a maize unknown protein (GenBank Accession: ACF80385.1). The top 10 homologous sequences identified in the BlastX query are presented in Table 35.

TABLE 35
BLASTX search results of the maize ZM3s00207 (MAWS27)

| | | | | % Iden- |
|---|---|---|---|---|
| Accession | Description | Score | E-value | tities |
| ACF80385.1 | unknown [Zea mays] | 155 | 6.00E−41 | 89 |
| BAA83559.1 | putative early nodulin | 155 | 1.00E−39 | 85 |
| | [Oryza sativa | | | |
| | Japonica Group] | | | |
| BAA83567.1 | putative early nodulin | 154 | 2.00E−39 | 84 |
| | [Oryza sativa | | | |
| | Japonica Group] | | | |
| NP_001056762.1 | Os06g0141700 [Oryza | 152 | 5.00E−39 | 85 |
| | sativa (japonica | | | |
| | cultivar-group)] | | | |
| NP_001056767.1 | Os06g0142300 [Oryza | 151 | 6.00E−39 | 85 |
| | sativa (japonica | | | |
| | cultivar-group)] | | | |
| BAA33813.1 | early nodulin | 154 | 6.00E−39 | 86 |
| | [Oryza sativa | | | |
| | Japonica Group] | | | |
| BAA83566.1 | putative early nodulin | 151 | 6.00E−39 | 85 |
| | [Oryza sativa | | | |
| | Japonica Group] | | | |
| EAY99605.1 | hypothetical protein | 154 | 6.00E−39 | 86 |
| | OsI_020838 [Oryza | | | |
| | sativa (indica | | | |
| | cultivar-group)] | | | |
| EAY99606.1 | hypothetical protein | 151 | 6.00E−39 | 85 |
| | OsI_020839 [Oryza | | | |
| | sativa (indica | | | |
| | cultivar-group)] | | | |
| EAY99601.1 | hypothetical protein | 149 | 1.00E−38 | 84 |
| | OsI_020834 [Oryza | | | |
| | sativa (indica | | | |
| | cultivar-group)] | | | |

The CDS sequence of the gene corresponding to MAWS27 is shown in SEQ ID NO: 24 and the translated amino acid sequence is shown in SEQ ID NO: 42.

### Identification of the Promoter Region of MAWS27

For our promoter identification purposes, the sequence upstream of the start codon of the MAWS27 gene was defined as the promoter p-MAWS27. To identify this predicted promoter region, the sequence of ZM3s00207 was mapped to the BASF Plant Science proprietary maize genomic DNA sequence database, PUB_tigr_maize_genomic_partial_{—}5.0.nt. One maize genomic DNA sequences, AZM5_{—}32720 (2113 bp, SEQ ID NO: 78) was identified. This 2113 bp sequence harbored the predicted CDS of the corresponding gene to MAWS27 and 1.2 kb upstream sequence of the ATG start codon of this gene. In addition, a public available sequence DX863447 was overlapped with AZM5_{—}32720. The contig of these 2 genomic sequences containing 1.35 kb upstream region is shown in SEQ ID NO: 78.

### Isolation of the Promoter Region by PCR Amplification

The putative promoter region was isolated via genomic PCR using the following sequence specific primers:

Forward primer:

(SEQ ID NO: 174)

TATAAATTAGAACGGAGGGGTATG
Reverse primer:

(SEQ ID NO: 175)

GGTGATCCGAATCCGATCCC.

The expected 1355 bp fragment was amplified from maize genomic DNA, and named as promoter MAWS27 (p-MAWS27). Sequence of p-MAWS27 is shown in SEQ ID NO: 6.
BLASTN Results of p_MAWS27

The top 20 homologous sequences identified in the BlastN query of p-MAWS27 are presented in Table 36.

TABLE 36
BLASTN results of p_MAWS27

| | | Max | Total | Query | | Max |
|---|---|---|---|---|---|---|
| Accession | Description | score | score | coverage | E value | ident |
| AY072300.1 | Zea mays cytochrome | 228 | 443 | 13% | 5.00E−56 | 90% |
| | P450 monooxygenase | | | | | |
| | CYP72A5 gene, complete | | | | | |
| | cds | | | | | |
| BT042628.1 | Zea mays full-length cDNA | 215 | 283 | 11% | 3.00E−52 | 93% |
| | clone ZM_BFb0383P13 | | | | | |
| | mRNA, complete cds | | | | | |
| BT041400.1 | Zea mays full-length cDNA | 210 | 273 | 13% | 1.00E−50 | 89% |
| | clone ZM_BFc0115C19 | | | | | |
| | mRNA, complete cds | | | | | |
| AJ251453.1 | Zea mays see2a gene for | 208 | 269 | 11% | 5.00E−50 | 90% |
| | putative legumain, exons | | | | | |
| | 1-9 | | | | | |
| EU241894.1 | Zea mays ZCN3 (ZCN3) | 199 | 271 | 13% | 3.00E−47 | 90% |
| | gene, complete cds | | | | | |
| EU943068.1 | Zea mays clone 1558247 | 197 | 265 | 13% | 9.00E−47 | 93% |
| | mRNA sequence | | | | | |
| EU953408.1 | Zea mays clone 1408713 | 190 | 255 | 11% | 1.00E−44 | 93% |
| | unknown mRNA | | | | | |
| AY662985.1 | Zea luxurians YZ1 (yz1) | 187 | 238 | 13% | 2.00E−43 | 88% |
| | gene, complete cds; | | | | | |
| | transposons mPIF-like | | | | | |
| | element and frequent flyer, | | | | | |
| | complete sequence; and | | | | | |
| | NADPH-dependent | | | | | |
| | reductase (a1) gene, | | | | | |
| | partial cds | | | | | |
| AJ437282.1 | Zea mays ZmEBE-2 gene | 176 | 176 | 11% | 3.00E−40 | 85% |
| | for ZmEBE-2 protein, | | | | | |
| | exons 1-4 | | | | | |
| AJ437281.1 | Zea mays ZmEBE-1 gene | 169 | 215 | 11% | 4.00E−38 | 85% |
| | for ZmEBE-1 protein, | | | | | |
| | exons 1-5 | | | | | |
| AY530950.1 | Zea mays putative zinc | 167 | 231 | 13% | 2.00E−37 | 93% |
| | finger protein (Z438D03.1), | | | | | |
| | unknown (Z438D03.5), | | | | | |
| | epsilon-COP (Z438D03.6), | | | | | |
| | putative kinase | | | | | |
| | (Z438D03.7), unknown | | | | | |
| | (Z438D03.25), and C1-B73 | | | | | |
| | (Z438D03.27) genes, | | | | | |
| | complete cds | | | | | |
| DQ020097.1 | Zea mays cultivar B73 | 165 | 226 | 13% | 5.00E−37 | 89% |
| | inbred aberrant pollen | | | | | |
| | transmission 1 (apt1) gene, | | | | | |
| | complete cds | | | | | |
| AY555143.1 | Zea may BAC clone | 163 | 309 | 13% | 2.00E−36 | 85% |
| | c573L14, complete | | | | | |
| | sequence | | | | | |
| AY111966.1 | Zea mays CL4954_1 | 158 | 206 | 10% | 8.00E−35 | 96% |
| | mRNA sequence | | | | | |
| EU975033.1 | Zea mays clone 465494 | 156 | 156 | 11% | 3.00E−34 | 83% |
| | unknown mRNA | | | | | |
| AF391808.3 | Zea mays cultivar McC bz | 156 | 272 | 17% | 3.00E−34 | 82% |
| | locus region | | | | | |
| U09989.1 | Zea mays D3L H(+)- | 156 | 218 | 11% | 3.00E−34 | 89% |
| | transporting ATPase | | | | | |
| | (Mha1) gene, complete cds | | | | | |
| EU241912.1 | Zea mays ZCN21 (ZCN21) | 154 | 204 | 11% | 1.00E−33 | 84% |
| | gene, complete cds | | | | | |
| BT039577.1 | Zea mays full-length cDNA | 149 | 197 | 12% | 4.00E−32 | 83% |
| | clone ZM_BFc0031C07 | | | | | |
| | mRNA, complete cds | | | | | |
| DQ219417.1 | Zea mays YZ1 (Yz1A) | 149 | 362 | 13% | 4.00E−32 | 89% |
| | gene, Yz1A-1012M allele, | | | | | |
| | partial cds; and a1 gene, | | | | | |
| | A1-b alpha allele, | | | | | |
| | transposon Ins2, and cin4 | | | | | |
| | retrotransposon, complete | | | | | |
| | sequence | | | | | |

### 3. Annotation of MAWS30

MAWS30 encodes maize 17 Kda oleosin (GenBank Accession: AAA68066.1). The top 10 homologous sequences identified in the BlastX query are presented in Table 37.

TABLE 37
BLASTX search results of the maize ZM1a61269071 (MAWS30)

| | | | | % Iden- |
|---|---|---|---|---|
| Accession | Description | Score | E-value | tities |
| AAA68066.1 | 17 kDa oleosin | 66 | 3.00E−21 | 100 |
| P21641 | OLEO3_MAIZE | 54 | 1.00E−15 | 95 |
| | Oleosin Zm-II | | | |
| | (Oleosin 18 kDa) | | | |
| | (Lipid body- | | | |
| | associated protein L2) | | | |
| CAA57994.1 | high molecular weight | 54 | 2.00E−14 | 77 |
| | oleosin [Hordeum | | | |
| | vulgare subsp. vulgare] | | | |
| CAN80217.1 | hypothetical protein | 81 | 2.00E−13 | 42 |
| | [Vitis vinifera] | | | |
| NP_001050984.1 | Os03g0699000 [Oryza | 48 | 3.00E−13 | 70 |
| | sativa (japonica | | | |
| | cultivar-group)] | | | |
| AAC02240.1 | 18 kDa oleosin | 48 | 3.00E−13 | 70 |
| | [Oryza sativa] | | | |
| AAG43516.1 | AF210696_1 15 kD | 39 | 8.00E−06 | 73 |
| | oleosin-like protein 1 | | | |
| | [Perilla frutescens] | | | |
| AAG43517.1 | AF210697_1 15 kD | 39 | 8.00E−06 | 73 |
| | oleosin-like protein 2 | | | |
| | [Perilla frutescens] | | | |
| AAB58402.1 | 15.5 kDa oleosin | 37 | 5.00E−05 | 66 |
| | [Sesamum indicum] | | | |
| CAN80922.1 | hypothetical protein | 36 | 7.00E−05 | 57 |
| | [Vitis vinifera] | | | |

The CDS sequence of the gene corresponding to MAWS30 is shown in SEQ ID NO: 34 and the translated amino acid sequence is shown in SEQ ID NO: 52.

### Identification of the Promoter Region of MAWS30

For our promoter identification purposes, the sequence upstream of the start codon of the MAWS30 gene was defined as the promoter p-MAWS30. To identify this predicted promoter region, the sequence of ZM1a61269071 was mapped to the BASF Plant Science proprietary maize genomic DNA sequence database, PUB_tigr_maize_genomic_partial_{—}5.0.nt. One maize genomic DNA sequences, AZM5_{—}84557 (3426 bp) was identified. This 3426 bp sequence harbored the predicted CDS of the corresponding gene to MAWS30 and about 0.6 kb upstream sequence of the ATG start codon of this gene. Sequence of AZM5_{—}84557 is shown in SEQ ID NO: 88.

### Isolation of the Promoter Region by PCR Amplification

The putative promoter region was isolated via genomic PCR using the following sequence specific primers:

Forward primer:

(SEQ ID NO: 176)

CTCACACAAATCTAAATAGTAAAG
Reverse primer:

(SEQ ID NO: 177)

GAGAGAGAGAGTAGTGAAGTG.

The expected 623 bp fragment was amplified from maize genomic DNA, and named as promoter MAWS30 (p-MAWS30). Sequence of p-MAWS30 was shown in SEQ ID NO: 16.
BLASTN Results of p_MAWS30

The top 20 homologous sequences identified in the BlastN query of p_MAWS30 are presented in Table 38.

TABLE 38
BLASTN results of p_MAWS30

| | | Max | Total | Query | | Max |
|---|---|---|---|---|---|---|
| Accession | Description | score | score | coverage | E value | ident |
| U13702.1 | Zea mays oil body protein 17 kDa | 686 | 686 | 60% | 0 | 100% |
| | oleosin (ole17) gene, | | | | | |
| | complete cds | | | | | |
| J05212.1 | Maize oleosin KD18 (KD18; L2) | 187 | 187 | 42% | 7.00E−44 | 75% |
| | gene, complete cds | | | | | |
| AY427563.1 | Oryza sativa (japonica cultivar- | 98.7 | 98.7 | 18% | 3.00E−17 | 78% |
| | group) 18 kDa oleosin gene, | | | | | |
| | promoter region | | | | | |
| AF019212.1 | Oryza sativa subsp. indica 18 kDa | 98.7 | 98.7 | 18% | 3.00E−17 | 78% |
| | oleosin (ole18) gene, | | | | | |
| | complete cds | | | | | |
| AP008209.1 | Oryza sativa (japonica cultivar- | 95.1 | 95.1 | 18% | 4.00E−16 | 77% |
| | group) genomic DNA, | | | | | |
| | chromosome 3 | | | | | |
| AC097368.3 | Oryza sativa chromosome 3 | 95.1 | 95.1 | 18% | 4.00E−16 | 77% |
| | BAC OSJNBb0017F17 genomic | | | | | |
| | sequence, complete sequence | | | | | |
| AF369906.1 | Sorghum bicolor clone | 53.6 | 53.6 | 8% | 0.001 | 82% |
| | BAC10J22 Sbb3766 sequence | | | | | |
| FJ119498.1 | Pinus taeda isolate 8102 | 46.4 | 46.4 | 6% | 0.16 | 85% |
| | anonymous locus | | | | | |
| | UMN_CL22Contig1_02 genomic | | | | | |
| | sequence | | | | | |
| FJ119497.1 | Pinus taeda isolate 8112 | 46.4 | 46.4 | 6% | 0.16 | 85% |
| | anonymous locus | | | | | |
| | UMN_CL22Contig1_02 genomic | | | | | |
| | sequence | | | | | |
| FJ119496.1 | Pinus taeda isolate 8099 | 46.4 | 46.4 | 6% | 0.16 | 85% |
| | anonymous locus | | | | | |
| | UMN_CL22Contig1_02 genomic | | | | | |
| | sequence | | | | | |
| FJ119495.1 | Pinus taeda isolate 8105 | 46.4 | 46.4 | 6% | 0.16 | 85% |
| | anonymous locus | | | | | |
| | UMN_CL22Contig1_02 genomic | | | | | |
| | sequence | | | | | |
| FJ119494.1 | Pinus taeda isolate 8108 | 46.4 | 46.4 | 6% | 0.16 | 85% |
| | anonymous locus | | | | | |
| | UMN_CL22Contig1_02 genomic | | | | | |
| | sequence | | | | | |
| FJ119493.1 | Pinus taeda isolate 8103 | 46.4 | 46.4 | 6% | 0.16 | 85% |
| | anonymous locus | | | | | |
| | UMN_CL22Contig1_02 genomic | | | | | |
| | sequence | | | | | |
| FJ119492.1 | Pinus taeda isolate 8100 | 46.4 | 46.4 | 6% | 0.16 | 85% |
| | anonymous locus | | | | | |
| | UMN_CL22Contig1_02 genomic | | | | | |
| | sequence | | | | | |
| FJ119491.1 | Pinus taeda isolate 8107 | 46.4 | 46.4 | 6% | 0.16 | 85% |
| | anonymous locus | | | | | |
| | UMN_CL22Contig1_02 genomic | | | | | |
| | sequence | | | | | |
| FJ119490.1 | Pinus taeda isolate 8113 | 46.4 | 46.4 | 6% | 0.16 | 85% |
| | anonymous locus | | | | | |
| | UMN_CL22Contig1_02 genomic | | | | | |
| | sequence | | | | | |
| FJ119489.1 | Pinus taeda isolate 8101 | 46.4 | 46.4 | 6% | 0.16 | 85% |
| | anonymous locus | | | | | |
| | UMN_CL22Contig1_02 genomic | | | | | |
| | sequence | | | | | |
| FJ119488.1 | Pinus taeda isolate 8111 | 46.4 | 46.4 | 6% | 0.16 | 85% |
| | anonymous locus | | | | | |
| | UMN_CL22Contig1_02 genomic | | | | | |
| | sequence | | | | | |
| FJ119487.1 | Pinus taeda isolate 8114 | 46.4 | 46.4 | 6% | 0.16 | 85% |
| | anonymous locus | | | | | |
| | UMN_CL22Contig1_02 genomic | | | | | |
| | sequence | | | | | |
| FJ119486.1 | Pinus taeda isolate 8110 | 46.4 | 46.4 | 6% | 0.16 | 85% |
| | anonymous locus | | | | | |
| | UMN_CL22Contig1_02 genomic | | | | | |
| | sequence | | | | | |

### 4. Annotation of MAWS57

MAWS57 encodes a protein that has homolog to a rice unknown protein Os05g0576700 (GenBank Accession: NP_{—}001056403.1). The top 10 homologous sequences identified in the BlastX query are presented in Table 39.

TABLE 39
BLASTX search results of the maize ZM1s57500283 (MAWS57)

| | | | | % Iden- |
|---|---|---|---|---|
| Accession | Description | Score | E-value | tities |
| NP_001056403.1 | Os05g0576700 [Oryza | 112 | 8.00E−33 | 95 |
| | sativa (japonica | | | |
| | cultivar-group)] | | | |
| ABK40507.1 | pollen oleosin | 102 | 2.00E−26 | 82 |
| | [Lilium longiflorum] | | | |
| EAZ35378.1 | hypothetical protein | 112 | 5.00E−25 | 95 |
| | OsJ_018861 [Oryza | | | |
| | sativa (japonica | | | |
| | cultivar-group)] | | | |
| AAX49393.1 | OLE-5 | 92 | 4.00E−19 | 81 |
| | [Coffea canephora] | | | |
| CAO68008.1 | unnamed protein | 89 | 2.00E−18 | 73 |
| | product [Vitis vinifera] | | | |
| NP_188487.1 | glycine-rich protein/ | 90 | 6.00E−16 | 78 |
| | oleosin | | | |
| | [Arabidopsis thaliana] | | | |
| ACI87763.1 | putative oleosin | 84 | 5.00E−14 | 69 |
| | [Cupressus | | | |
| | sempervirens] | | | |
| ACA30297.1 | putative oleosin | 84 | 5.00E−14 | 69 |
| | [Cupressus | | | |
| | sempervirens] | | | |
| NP_175329.1 | glycine-rich protein/ | 74 | 5.00E−11 | 69 |
| | oleosin | | | |
| | [Arabidopsis thaliana] | | | |
| CAN74835.1 | hypothetical protein | 66 | 1.00E−08 | 53 |
| | [Vitis vinifera] | | | |

The CDS sequence of the gene corresponding to MAWS57 is shown in SEQ ID NO: 35 and the translated amino acid sequence is shown in SEQ ID NO: 55.

### Identification of the Promoter Region of MAWS57

For our promoter identification purposes, the sequence upstream of the start codon of the MAWS57 gene was defined as the promoter p-MAWS57. To identify this predicted promoter region, the sequence of ZM1s57500283 was mapped to the BASF Plant Science proprietary maize genomic DNA sequence database, PUB_tigr_maize_genomic_partial_{—}5.0.nt. One maize genomic DNA sequences, AZM5_{—}16632 (5254 bp) was identified. This 5254 bp sequence harbored the predicted CDS of the corresponding gene to MAWS57 and more than 2.5 kb upstream sequence of the ATG start codon of this gene. Sequences of AZM5_{—}16632 is shown in SEQ ID NO: 89.

### Isolation of the Promoter Region by PCR Amplification

The putative promoter region was isolated via genomic PCR using the following sequence specific primers:

Forward primer:

(SEQ ID NO: 178)

GGTTCAAGATATGTATGTGATG
Reverse primer:

(SEQ ID NO: 179)

TCGGGTATCTCTCTGTCTTGTTG.

The expected 1950 bp fragment was amplified from maize genomic DNA, and named as promoter MAWS57 (p-MAWS57). Sequence of p-MAWS57 was shown in SEQ ID NO: 17.
BLASTN Results of p_MAWS57

The top 20 homologous sequences identified in the BlastN query of p_MAWS57 are presented in Table 40.

TABLE 40
BLASTN results of p_MAWS57

| | | Max | Total | Query | | Max |
|---|---|---|---|---|---|---|
| Accession | Description | score | score | coverage | E value | ident |
| AP008217.1 | Oryza sativa (japonica | 259 | 415 | 26% | 5.00E−65 | 81% |
| | cultivar-group) genomic | | | | | |
| | DNA, chromosome 11 | | | | | |
| BX000501.4 | Oryza sativa | 259 | 415 | 26% | 5.00E−65 | 81% |
| | chromosome 11, . BAC | | | | | |
| | OSJNBa0032J07 of | | | | | |
| | library OSJNBa from | | | | | |
| | chromosome 11 of | | | | | |
| | cultivar Nipponbare of | | | | | |
| | ssp. japonica of Oryza | | | | | |
| | sativa (rice), complete | | | | | |
| | sequence | | | | | |
| AP008218.1 | Oryza sativa (japonica | 179 | 447 | 23% | 4.00E−41 | 83% |
| | cultivar-group) genomic | | | | | |
| | DNA, chromosome 12 | | | | | |
| BX000494.2 | Oryza sativa | 179 | 447 | 23% | 4.00E−41 | 83% |
| | chromosome 12, . BAC | | | | | |
| | OSJNBa0052H10 of | | | | | |
| | library OSJNBa from | | | | | |
| | chromosome 12 of | | | | | |
| | cultivar Nipponbare of | | | | | |
| | ssp. japonica of Oryza | | | | | |
| | sativa (rice), complete | | | | | |
| | sequence | | | | | |
| BX000491.1 | Oryza sativa | 179 | 447 | 23% | 4.00E−41 | 83% |
| | chromosome 12, . BAC | | | | | |
| | OSJNBb0068K19 of | | | | | |
| | library OSJNBb from | | | | | |
| | chromosome 12 of | | | | | |
| | cultivar Nipponbare of | | | | | |
| | ssp. japonica of Oryza | | | | | |
| | sativa (rice), complete | | | | | |
| | sequence | | | | | |
| AK242870.1 | Oryza sativa Japonica | 176 | 389 | 18% | 4.00E−40 | 84% |
| | Group cDNA, clone: | | | | | |
| | J090076L04, full insert | | | | | |
| | sequence | | | | | |
| NM_001072463.1 | Oryza sativa (japonica | 167 | 389 | 18% | 2.00E−37 | 84% |
| | cultivar-group) | | | | | |
| | Os12g0105300 | | | | | |
| | (Os12g0105300) mRNA, | | | | | |
| | complete cds | | | | | |
| AK099132.1 | Oryza sativa Japonica | 167 | 389 | 18% | 2.00E−37 | 84% |
| | Group cDNA | | | | | |
| | clone: J023051M04, full | | | | | |
| | insert sequence | | | | | |
| AK072914.1 | Oryza sativa Japonica | 167 | 389 | 18% | 2.00E−37 | 84% |
| | Group cDNA | | | | | |
| | clone: J023150I16, full | | | | | |
| | insert sequence | | | | | |
| AK062121.1 | Oryza sativa Japonica | 167 | 389 | 18% | 2.00E−37 | 84% |
| | Group cDNA clone: 001- | | | | | |
| | 045-D01, full insert | | | | | |
| | sequence | | | | | |
| AK250796.1 | Hordeum vulgare subsp. | 123 | 323 | 25% | 2.00E−24 | 84% |
| | vulgare cDNA clone: | | | | | |
| | FLbaf94j01, mRNA | | | | | |
| | sequence | | | | | |
| AP008213.1 | Oryza sativa (japonica | 111 | 111 | 5% | 2.00E−20 | 81% |
| | cultivar-group) genomic | | | | | |
| | DNA, chromosome 7 | | | | | |
| AP005768.3 | Oryza sativa Japonica | 111 | 111 | 5% | 2.00E−20 | 81% |
| | Group genomic DNA, | | | | | |
| | chromosome 7, BAC | | | | | |
| | clone: OSJNBa0039C01 | | | | | |
| AP005255.4 | Oryza sativa Japonica | 111 | 111 | 5% | 2.00E−20 | 81% |
| | Group genomic DNA, | | | | | |
| | chromosome 7, BAC | | | | | |
| | clone: OSJNBb0087F05 | | | | | |
| AC232448.1 | Brassica rapa subsp. | 100 | 191 | 9% | 3.00E−17 | 93% |
| | pekinensis clone | | | | | |
| | KBrB008D15, complete | | | | | |
| | sequence | | | | | |
| CT828672.1 | Oryza sativa (indica | 98.7 | 141 | 6% | 1.00E−16 | 87% |
| | cultivar-group) cDNA | | | | | |
| | clone: OSIGCSA057D18, | | | | | |
| | full insert sequence | | | | | |
| AM448932.2 | Vitis vinifera contig | 96.9 | 177 | 15% | 3.00E−16 | 79% |
| | VV78X114050.3, whole | | | | | |
| | genome shotgun | | | | | |
| | sequence | | | | | |
| AP010508.1 | Lotus japonicus genomic | 95.1 | 241 | 16% | 1.00E−15 | 90% |
| | DNA, chromosome 2, | | | | | |
| | clone: LjT28N02, | | | | | |
| | TM1615, complete | | | | | |
| | sequence | | | | | |
| EF145201.1 | Populus trichocarpa | 87.8 | 155 | 9% | 2.00E−13 | 85% |
| | clone WS01121_K10 | | | | | |
| | unknown mRNA | | | | | |
| AC098571.2 | Oryza sativa Japonica | 80.6 | 80.6 | 3% | 3.00E−11 | 84% |
| | Group chromosome 5 | | | | | |
| | clone OJ1126_B10, | | | | | |
| | complete sequence | | | | | |

### 5. Annotation of MAWS60

MAWS60 encodes a maize unknown protein (GenBank Accession: ACF78165.1). The top 10 homologous sequences identified in the BlastX query are presented in Table 41.

TABLE 41
BLASTX search results of the maize ZM4s20063 (MAWS60)

| | | | | % Iden- |
|---|---|---|---|---|
| Accession | Description | Score | E-value | tities |
| ACF78165.1 | unknown [Zea mays] | 138 | 3.00E−52 | 64 |
| ACF83516.1 | unknown [Zea mays] | 204 | 2.00E−50 | 82 |
| ACF86030.1 | unknown [Zea mays] | 124 | 2.00E−48 | 73 |
| ACF87441.1 | unknown [Zea mays] | 79 | 1.00E−46 | 73 |
| ACF78865.1 | unknown [Zea mays] | 102 | 1.00E−22 | 72 |
| ACF88449.1 | unknown [Zea mays] | 42 | 5.00E−11 | 48 |
| NP_001066367.1 | Os12g0198700 [Oryza | 42 | 1.00E−10 | 46 |
| | sativa (japonica | | | |
| | cultivar-group)] | | | |
| NP_001066495.1 | Os12g0247700 [Oryza | 39 | 4.00E−10 | 61 |
| | sativa (japonica | | | |
| | cultivar-group)] | | | |
| ABR25456.1 | beta-glucosidase | 46 | 7.00E−10 | 53 |
| | aggregating factor | | | |
| | precursor [Oryza sativa | | | |
| | (indica cultivar-group)] | | | |
| NP_001066435.1 | Os12g0227500 [Oryza | 46 | 7.00E−10 | 53 |
| | sativa (japonica | | | |
| | cultivar-group)] | | | |

The CDS sequence of the gene corresponding to MAWS60 is shown in SEQ ID NO: 19 and the translated amino acid sequence is shown in SEQ ID NO: 37.

### Identification of the Promoter Region of MAWS60

For our promoter identification purposes, the sequence upstream of the start codon of the MAWS60 gene was defined as the promoter p-MAWS60. To identify this predicted promoter region, the sequence of ZM4s20063 was mapped to the BASF Plant Science proprietary maize genomic DNA sequence database, PUB_tigr_maize_genomic_partial_{—}5.0.nt. One maize genomic DNA sequences, AZM5_{—}25938 (3185 bp) was identified. This 3185 bp sequence harbored the predicted CDS of the corresponding gene to MAWS60 and 1.2 kb upstream. sequence of the ATG start codon of this gene. Sequence of AZM5_{—}25938 is shown in SEQ ID NO: 73.

### Isolation of the Promoter Region by PCR Amplification

The putative promoter region was isolated via genomic PCR using the following sequence specific primers:

Forward primer:

(SEQ ID NO: 180)

TTGGTTTTTTGATAATTTGTTTATC
Reverse primer:

(SEQ ID NO: 181)

TCTCCATTACCTGCAACGATC.

The expected 1106 bp fragment was amplified from maize genomic DNA, and named as promoter MAWS60 (p-MAWS60). Sequence of p-MAWS60 was shown in SEQ ID NO: 1.
BLASTN Results of p_MAWS60

The top 20 homologous sequences identified in the BlastN query of p_MAWS60 are presented in Table 42.

TABLE 42
BLASTN results of p_MAWS60

| | | Max | Total | Query | | Max |
|---|---|---|---|---|---|---|
| Accession | Description | score | score | coverage | E value | ident |
| AC157320.2 | Zea mays clone | 452 | 801 | 44% | 2.00E−123 | 79% |
| | ZMMBBb-7C14, | | | | | |
| | complete sequence | | | | | |
| AF544161.1 | Zea mays subsp. mays | 320 | 320 | 22% | 9.00E−84 | 89% |
| | cultivar A6 ADP-glucose | | | | | |
| | pyrophosphorylase large | | | | | |
| | subunit (shrunken2) | | | | | |
| | gene, partial sequence | | | | | |
| AF544159.1 | Zea mays subsp. mays | 320 | 320 | 22% | 9.00E−84 | 89% |
| | cultivar Tx601 ADP- | | | | | |
| | glucose | | | | | |
| | pyrophosphorylase large | | | | | |
| | subunit (shrunken2) | | | | | |
| | gene, partial sequence | | | | | |
| AF544158.1 | Zea mays subsp. mays | 320 | 320 | 22% | 9.00E−84 | 89% |
| | cultivar Ki9 ADP-glucose | | | | | |
| | pyrophosphorylase large | | | | | |
| | subunit (shrunken2) | | | | | |
| | gene, partial sequence | | | | | |
| AF544157.1 | Zea mays subsp. mays | 320 | 320 | 22% | 9.00E−84 | 89% |
| | cultivar T232 ADP- | | | | | |
| | glucose | | | | | |
| | pyrophosphorylase large | | | | | |
| | subunit (shrunken2) | | | | | |
| | gene, partial sequence | | | | | |
| U07956.1 | Zea mays transposable | 320 | 320 | 22% | 9.00E−84 | 89% |
| | element ILS-1 | | | | | |
| AC165178.2 | Zea mays clone | 315 | 860 | 32% | 4.00E−82 | 84% |
| | ZMMBBb-272P17, | | | | | |
| | complete sequence | | | | | |
| AF544160.1 | Zea mays subsp. mays | 315 | 315 | 22% | 4.00E−82 | 88% |
| | cultivar A272 ADP- | | | | | |
| | glucose | | | | | |
| | pyrophosphorylase large | | | | | |
| | subunit (shrunken2) | | | | | |
| | gene, partial sequence | | | | | |
| AC160211.1 | Genomic seqeunce for | 279 | 428 | 41% | 3.00E−71 | 86% |
| | Zea mays BAC clone | | | | | |
| | ZMMBBb0448F23, | | | | | |
| | complete sequence | | | | | |
| AY530950.1 | Zea mays putative zinc | 264 | 541 | 34% | 6.00E−67 | 82% |
| | finger protein | | | | | |
| | (Z438D03.1), unknown | | | | | |
| | (Z438D03.5), epsilon- | | | | | |
| | COP (Z438D03.6), | | | | | |
| | putative kinase | | | | | |
| | (Z438D03.7), unknown | | | | | |
| | (Z438D03.25), and C1- | | | | | |
| | B73 (Z438D03.27) | | | | | |
| | genes, complete cds | | | | | |
| AF061282.1 | Sorghum bicolor 22 kDa | 179 | 429 | 35% | 2.00E−41 | 96% |
| | kafirin cluster | | | | | |
| AY661657.1 | Sorghum bicolor clone | 167 | 167 | 25% | 1.00E−37 | 74% |
| | BAC 60H10, complete | | | | | |
| | sequence | | | | | |
| AY661656.1 | Sorghum bicolor clone | 167 | 535 | 35% | 1.00E−37 | 88% |
| | BAC 88M4, complete | | | | | |
| | sequence | | | | | |
| AC169377.4 | Sorghum bicolor clone | 154 | 154 | 17% | 8.00E−34 | 79% |
| | SB_BBc0068O12, | | | | | |
| | complete sequence | | | | | |
| AC169379.4 | Sorghum bicolor clone | 154 | 154 | 17% | 8.00E−34 | 79% |
| | SB_BBc0088B22, | | | | | |
| | complete sequence | | | | | |
| AP008208.1 | Oryza sativa (japonica | 131 | 4754 | 22% | 9.00E−27 | 78% |
| | cultivar-group) genomic | | | | | |
| | DNA, chromosome 2 | | | | | |
| AP005066.2 | Oryza sativa Japonica | 131 | 218 | 22% | 9.00E−27 | 75% |
| | Group genomic DNA, | | | | | |
| | chromosome 2, PAC | | | | | |
| | clone: P0047E05 | | | | | |
| AY144442.1 | Sorghum bicolor BAC | 127 | 766 | 19% | 1.00E−25 | 88% |
| | 95A23/98N8.1 Rph | | | | | |
| | region, partial sequence | | | | | |
| AP008218.1 | Oryza sativa (japonica | 125 | 6385 | 22% | 4.00E−25 | 80% |
| | cultivar-group) genomic | | | | | |
| | DNA, chromosome 12 | | | | | |
| AL831796.5 | Oryza sativa | 125 | 125 | 22% | 4.00E−25 | 71% |
| | chromosome 12, . BAC | | | | | |
| | OSJNBa0012G19 of | | | | | |
| | library OSJNBa from | | | | | |
| | chromosome 12 of | | | | | |
| | cultivar Nipponbare of | | | | | |
| | ssp. japonica of Oryza | | | | | |
| | sativa (rice), complete | | | | | |
| | sequence | | | | | |

### 6. Annotation of MAWS63

MAWS63 encodes a protein that is homologous to a rice hypothetical protein Osl_{—}026531 (GenBank Accession: EAZ05299.1). The top 10 homologous sequences identified in the BlastX query are presented in Table 43.

TABLE 43
BLASTX search results of the maize ZM1s62013293 (MAWS63)

| | | | | % Iden- |
|---|---|---|---|---|
| Accession | Description | Score | E-value | tities |
| EAZ05299.1 | hypothetical protein | 98 | 2.00E−20 | 59 |
| | OsI_026531 | | | |
| NP_001060778.1 | Os08g0104400 [Oryza | 94 | 2.00E−19 | 72 |
| | sativa (japonica | | | |
| | cultivar-group)] | | | |
| EAZ05298.1 | hypothetical protein | 99 | 9.00E−19 | 53 |
| | OsI_026530 [Oryza | | | |
| | sativa (indica | | | |
| | cultivar-group)] | | | |
| CAO22190.1 | unnamed protein | 58 | 2.00E−06 | 61 |
| | product [Vitis vinifera] | | | |
| CAO46216.1 | unnamed protein | 58 | 2.00E−06 | 61 |
| | product [Vitis vinifera] | | | |
| CAN64204.1 | hypothetical protein | 58 | 2.00E−06 | 61 |
| | [Vitis vinifera] | | | |
| ABB72396.1 | seed maturation | 58 | 2.00E−06 | 62 |
| | protein [Glycine | | | |
| | tomentella] | | | |
| ABB72388.1 | seed maturation | 58 | 2.00E−06 | 62 |
| | protein [Glycine | | | |
| | latifolia] | | | |
| ABB72387.1 | seed maturation | 58 | 2.00E−06 | 62 |
| | protein [Glycine | | | |
| | latifolia] | | | |
| ABB72392.1 | seed maturation | 58 | 2.00E−06 | 62 |
| | protein [Glycine | | | |
| | tomentella] | | | |

The CDS sequence of the gene corresponding to MAWS63 is shown in SEQ ID NO: 25 and the translated amino acid sequence is shown in SEQ ID NO: 43.

### Identification of the Promoter Region of MAWS63

For our promoter identification purposes, the sequence upstream of the start codon of the MAWS63 gene was defined as the promoter p-MAWS63. To identify this predicted promoter region, the sequence of ZM1s62013293 was mapped to the BASF Plant Science proprietary maize genomic DNA sequence database, PUB_tigr_maize_genomic_partial_{—}5.0.nt. One maize genomic DNA sequences, AZM5_{—}12462 (5275 bp) was identified. This 5275 bp sequence harbored the predicted CDS of the corresponding gene to MAWS63 and 2.3 kb upstream sequence of the ATG start codon of this gene. The first 3 kb sequence of AZM5_{—}12462 is shown in SEQ ID NO: 79.

### Isolation of the Promoter Region by PCR Amplification

The putative promoter region was isolated via genomic PCR using the following sequence specific primers:

Forward primer:

(SEQ ID NO: 182)

AAGGGACTCGTGGCCTACAC
Reverse primer:

(SEQ ID NO: 183)

TACGTTGTCGCAGCTGGATG.

The expected 1941 bp fragment was amplified from maize genomic DNA, and named as promoter MAWS63 (p-MAWS63). Sequence of p-MAWS63 is shown in SEQ ID NO: 7.
BLASTN Results of p_MAWS63

The top 20 homologous sequences identified in the BlastN query of p_MAWS63 are presented in Table 44.

TABLE 44
BLASTN results of p_MAWS63

| | | Max | Total | Query | | Max |
|---|---|---|---|---|---|---|
| Accession | Description | score | score | coverage | E value | ident |
| BT040012.1 | Zea mays full-length cDNA | 1370 | 1370 | 39% | 0 | 100% |
| | clone ZM_BFc0058K21 | | | | | |
| | mRNA, complete cds | | | | | |
| EU967309.1 | Zea mays clone 301323 | 1308 | 1308 | 37% | 0 | 100% |
| | unknown mRNA | | | | | |
| BT043342.1 | Zea mays full-length cDNA | 1301 | 1301 | 37% | 0 | 100% |
| | clone ZM_BFc0164P09 | | | | | |
| | mRNA, complete cds | | | | | |
| DQ245437.1 | Zea mays clone 15518 | 1148 | 1148 | 33% | 0 | 99% |
| | mRNA sequence | | | | | |
| AY103722.1 | Zea mays PCO142214 | 1083 | 1083 | 38% | 0 | 91% |
| | mRNA sequence | | | | | |
| NM_001111875.1 | Zea mays ferredoxin1 | 1074 | 1074 | 39% | 0 | 90% |
| | (fdx1), nuclear gene | | | | | |
| | encoding mitochondrial | | | | | |
| | protein, mRNA | | | | | |
| | >gb\|M73830.1\|MZEFD1P | | | | | |
| | Maize ferredoxin I (Fd) | | | | | |
| | isoprotein mRNA, pFD1′ | | | | | |
| M73829.1 | Maize ferredoxin I (Fd) | 1027 | 1027 | 33% | 0 | 94% |
| | isoprotein mRNA, pFD1 | | | | | |
| EU328185.1 | Zea mays chloroplast | 812 | 812 | 23% | 0 | 99% |
| | ferredoxin 1 precursor | | | | | |
| | (FDX1) mRNA, complete | | | | | |
| | cds; nuclear gene for | | | | | |
| | chloroplast product | | | | | |
| EU328184.1 | Zea mays chloroplast | 545 | 545 | 22% | 2.00E−151 | 87% |
| | ferredoxin 5 precursor | | | | | |
| | (FDX5) mRNA, complete | | | | | |
| | cds; nuclear gene for | | | | | |
| | chloroplast product | | | | | |
| EU975349.1 | Zea mays clone 488257 | 542 | 542 | 22% | 3.00E−150 | 86% |
| | unknown mRNA | | | | | |
| EU972749.1 | Zea mays clone 387187 | 526 | 526 | 21% | 2.00E−145 | 87% |
| | unknown mRNA | | | | | |
| NM_001111874.1 | Zea mays ferredoxin5 | 495 | 495 | 22% | 4.00E−136 | 83% |
| | (fdx5), mRNA | | | | | |
| | >gb\|M73828.1\|MZEFD5 | | | | | |
| | Maize ferredoxin (Fd) | | | | | |
| | isoprotein mRNA, pFD5 | | | | | |
| NM_001111374.1 | Zea mays ferredoxin2 | 443 | 443 | 15% | 2.00E−120 | 92% |
| | (fdx2), mRNA | | | | | |
| | >dbj\|AB016810.1\|Zea | | | | | |
| | mays mRNA for ferredoxin, | | | | | |
| | complete cds | | | | | |
| BT039722.1 | Zea mays full-length cDNA | 434 | 434 | 15% | 1.00E−117 | 92% |
| | clone ZM_BFc0041B09 | | | | | |
| | mRNA, complete cds | | | | | |
| EU328186.1 | Zea mays chloroplast | 434 | 434 | 15% | 1.00E−117 | 92% |
| | ferredoxin 2 precursor | | | | | |
| | (FDX2) mRNA, complete | | | | | |
| | cds; nuclear gene for | | | | | |
| | chloroplast product | | | | | |
| EU974838.1 | Zea mays clone 459526 | 430 | 430 | 15% | 1.00E−116 | 91% |
| | unknown mRNA | | | | | |
| CT841984.1 | Oryza rufipogon (W1943) | 405 | 405 | 15% | 5.00E−109 | 89% |
| | cDNA clone: | | | | | |
| | ORW1943C104F01, full | | | | | |
| | insert sequence | | | | | |
| AK287537.1 | Oryza sativa Japonica | 405 | 405 | 15% | 5.00E−109 | 89% |
| | Group cDNA, clone: | | | | | |
| | J065007C21, full insert | | | | | |
| | sequence | | | | | |
| CU406957.1 | Oryza rufipogon (W1943) | 405 | 405 | 15% | 5.00E−109 | 89% |
| | cDNA clone: | | | | | |
| | ORW1943C107I19, full | | | | | |
| | insert sequence | | | | | |
| CU406556.1 | Oryza rufipogon (W1943) | 405 | 405 | 15% | 5.00E−109 | 89% |
| | cDNA clone: | | | | | |
| | ORW1943S102N16, full | | | | | |
| | insert sequence | | | | | |

### 7. Annotation of MAEM1

MAEM1 encodes maize ZCN9 protein (GenBank Accession: ABX11011.1). The top 10 homologous sequences identified in the BlastX query are presented in Table 45.

TABLE 45
BLASTX search results of the maize ZM4s09689 (MAEM1)

| | | | | % Iden- |
|---|---|---|---|---|
| Accession | Description | Score | E-value | tities |
| ABX11011.1 | ZCN9 [Zea mays] | 326 | 3.00E−97 | 100 |
| NP_001106248.1 | ZCN9 protein | 326 | 3.00E−97 | 100 |
| | [Zea mays] | | | |
| NP_001106249.1 | ZCN10 protein | 309 | 5.00E−92 | 94 |
| | [Zea mays] | | | |
| EAY84662.1 | hypothetical protein | 210 | 6.00E−76 | 84 |
| | OsI_005895 [Oryza | | | |
| | sativa (indica | | | |
| | cultivar-group)] | | | |
| NP_001041806.1 | Os01g0111600 [Oryza | 208 | 7.00E−75 | 83 |
| | sativa (japonica | | | |
| | cultivar-group)] | | | |
| NP_001057701.1 | Os06g0498800 [Oryza | 193 | 1.00E−65 | 68 |
| | sativa (japonica | | | |
| | cultivar-group)] | | | |
| ABB90591.1 | terminal flower 1 | 212 | 8.00E−60 | 63 |
| | [Aquilegia formosa] | | | |
| CAO68168.1 | unnamed protein | 170 | 2.00E−59 | 65 |
| | product [Vitis vinifera] | | | |
| BAD22677.1 | flowering locus T like | 173 | 5.00E−59 | 67 |
| | protein [Populus nigra] | | | |
| CAN80336.1 | hypothetical protein | 170 | 9.00E−59 | 65 |
| | [Vitis vinifera] | | | |

The CDS sequence of the gene corresponding to MAEM1 is shown in SEQ ID NO: 20 and the translated amino acid sequence is shown in SEQ ID NO: 38.

### Identification of the Promoter Region of MAEM1

For our promoter identification purposes, the sequence upstream of the start codon of the MAEM1 gene was defined as the promoter p-MAEM1. To identify this predicted promoter region, the sequence of ZM4s09689 was mapped to the BASF Plant Science proprietary maize genomic DNA sequence database, PUB_tigr_maize_genomic_partial_{—}5.0.nt. One maize genomic DNA sequences, AZM5_{—}13765 (3272 bp) was identified. This 3272 bp sequence harbored the predicted CDS of the corresponding gene to MAWS23 and less than 0.5 kb upstream sequence of the ATG start codon of this gene. In addition, a public available sequence CL383739 was overlapped with AZM5_{—}13765. The contig of these 2 genomic sequences containing 0.9 kb upstream region is shown in SEQ ID NO: 74.

### Isolation of the Promoter Region by PCR Amplification

The putative promoter region was isolated via genomic PCR using the following sequence specific primers:

Forward primer:

(SEQ ID NO: 184)

TTAGTAGAGAATAACACACATC
Reverse primer:

(SEQ ID NO: 185)

GATCGATCGATCAACGCG.

The expected 922 bp fragment was amplified from maize genomic DNA, and named as promoter MAEM1 (p-MAEM1). Sequence of p-MAEM1 was shown in SEQ ID NO: 2.
BLASTN Results of p_MAEM1

The top 18 homologous sequences identified in the BlastN query of p_MAEM1 are presented in Table46.

TABLE 46
BLASTN results of p_MAEM1

| | | Max | Total | Query | | Max |
|---|---|---|---|---|---|---|
| Accession | Description | score | score | coverage | E value | ident |
| EU241901.1 | Zea mays ZCN10 | 1651 | 1651 | 100% | 0 | 99% |
| | (ZCN10) gene, complete | | | | | |
| | cds | | | | | |
| NM_001112778.1 | Zea mays ZCN10 protein | 446 | 446 | 27% | 7.00E−122 | 99% |
| | (ZCN10), mRNA | | | | | |
| | >gb\|EU241926.1\|Zea | | | | | |
| | mays ZCN10 (ZCN10) | | | | | |
| | mRNA, complete cds | | | | | |
| AY530950.1 | Zea mays putative zinc | 208 | 208 | 39% | 3.00E−50 | 74% |
| | finger protein | | | | | |
| | (Z438D03.1), unknown | | | | | |
| | (Z438D03.5), epsilon- | | | | | |
| | COP (Z438D03.6), | | | | | |
| | putative kinase | | | | | |
| | (Z438D03.7), unknown | | | | | |
| | (Z438D03.25), and C1- | | | | | |
| | B73 (Z438D03.27) | | | | | |
| | genes, complete cds | | | | | |
| EU952257.1 | Zea mays clone | 196 | 196 | 11% | 2.00E−46 | 100% |
| | 1273313 unknown | | | | | |
| | mRNA | | | | | |
| EU241900.1 | Zea mays ZCN9 (ZCN9) | 176 | 176 | 19% | 2.00E−40 | 82% |
| | gene, complete cds | | | | | |
| AP008207.1 | Oryza sativa (japonica | 57.2 | 57.2 | 11% | 1.00E−04 | 72% |
| | cultivar-group) genomic | | | | | |
| | DNA, chromosome 1 | | | | | |
| AP004821.4 | Oryza sativa Japonica | 57.2 | 57.2 | 11% | 1.00E−04 | 72% |
| | Group genomic DNA, | | | | | |
| | chromosome 1, PAC | | | | | |
| | clone: P0676G08 | | | | | |
| AP003854.2 | Oryza sativa Japonica | 57.2 | 57.2 | 11% | 1.00E−04 | 72% |
| | Group genomic DNA, | | | | | |
| | chromosome 1, BAC | | | | | |
| | clone: OSJNBb0093M23 | | | | | |
| EU976588.1 | Zea mays clone 984310 | 55.4 | 55.4 | 3% | 5.00E−04 | 100% |
| | unknown mRNA | | | | | |
| AC135864.5 | Oryza sativa Japonica | 53.6 | 53.6 | 13% | 0.002 | 70% |
| | Group chromosome 11 | | | | | |
| | clone OSJNBb0071K13, | | | | | |
| | complete sequence | | | | | |
| AP008217.1 | Oryza sativa (japonica | 53.6 | 99 | 13% | 0.002 | 77% |
| | cultivar-group) genomic | | | | | |
| | DNA, chromosome 11 | | | | | |
| AP008215.1 | Oryza sativa (japonica | 51.8 | 187 | 5% | 0.006 | 85% |
| | cultivar-group) genomic | | | | | |
| | DNA, chromosome 9 | | | | | |
| AP005767.3 | Oryza sativa Japonica | 51.8 | 51.8 | 4% | 0.006 | 85% |
| | Group genomic DNA, | | | | | |
| | chromosome 9, BAC | | | | | |
| | clone: OSJNBa0035G04 | | | | | |
| AP005780.2 | Oryza sativa Japonica | 51.8 | 51.8 | 4% | 0.006 | 85% |
| | Group genomic DNA, | | | | | |
| | chromosome 9, BAC | | | | | |
| | clone: OSJNBb0051H02 | | | | | |
| AC139170.2 | Oryza sativa Japonica | 46.4 | 46.4 | 6% | 0.25 | 77% |
| | Group chromosome 11 | | | | | |
| | clone OSJNBa0058P12, | | | | | |
| | complete sequence | | | | | |
| AP008218.1 | Oryza sativa (japonica | 46.4 | 46.4 | 6% | 0.25 | 77% |
| | cultivar-group) genomic | | | | | |
| | DNA, chromosome 12 | | | | | |
| AP008208.1 | Oryza sativa (japonica | 46.4 | 92.7 | 5% | 0.25 | 81% |
| | cultivar-group) genomic | | | | | |
| | DNA, chromosome 2 | | | | | |
| AP005559.3 | Oryza sativa Japonica | 46.4 | 46.4 | 5% | 0.25 | 81% |
| | Group genomic DNA, | | | | | |
| | chromosome 9, BAC | | | | | |
| | clone: OJ1163_C07 | | | | | |

### 8. Annotation of MAEM20

MAEM20 encodes a protein that is homologous to rice hypothetical protein OsJ_{—}029225 (GenBank Accession: EAZ45742.1). The top 10 homologous sequences identified in the BlastX query are presented in Table 47.

TABLE 47
BLASTX search results of the maize ZM1s59153555 (MAEM20)

| | | | | % Iden- |
|---|---|---|---|---|
| Accession | Description | Score | E-value | tities |
| EAZ45742.1 | hypothetical protein | 154 | 4.00E−35 | 80 |
| | OsJ_029225 | | | |
| EAZ10155.1 | hypothetical protein | 154 | 4.00E−35 | 80 |
| | OsI_031387 [Oryza | | | |
| | sativa (indica | | | |
| | cultivar-group)] | | | |
| BAD46602.1 | putative Histone H2B | 154 | 4.00E−35 | 80 |
| | [Oryza sativa Japonica | | | |
| | Group] | | | |
| CAN78957.1 | hypothetical protein | 139 | 1.00E−30 | 69 |
| | [Vitis vinifera] | | | |
| NP_172295.1 | histone H2B family | 133 | 7.00E−30 | 62 |
| | protein [Arabidopsis | | | |
| | thaliana] | | | |
| XP_001104238.1 | PREDICTED: similar to | 132 | 2.00E−28 | 64 |
| | Histone H2B [Macaca | | | |
| | mulatta] | | | |
| XP_001914780.1 | PREDICTED: similar to | 131 | 4.00E−28 | 63 |
| | histone H2B.3 [Equus | | | |
| | caballus] | | | |
| XP_532763.2 | PREDICTED: similar to | 129 | 1.00E−27 | 62 |
| | testis-specific histone 2b | | | |
| | [Canis familiaris] | | | |
| XP_872016.1 | PREDICTED: similar to | 128 | 2.00E−27 | 62 |
| | histone H2B.3 [Bos | | | |
| | taurus] | | | |
| XP_002060453.1 | GJ19809 [Drosophila | 126 | 1.00E−26 | 59 |
| | virilis] | | | |

The CDS sequence of the gene corresponding to MAEM20 is shown in SEQ ID NO: 23 and the translated amino acid sequence is shown in SEQ ID NO: 41.

### Identification of the Promoter Region of MAEM20

For our promoter identification purposes, the sequence upstream of the start codon of the MAEM20 gene was defined as the promoter p-MAEM20. To identify this predicted promoter region, the sequence of ZM1s59153555 was mapped to the BASF Plant Science proprietary maize genomic DNA sequence database, PUB_tigr_maize_genomic_partial_{—}5.0.nt. One maize genomic DNA sequences, AZM5_{—}23292 (1996 bp) was identified. This 1996 bp sequence harbored the predicted CDS of the corresponding gene to MAEM20 and 0.7 kb upstream sequence of the ATG start codon of this gene. Sequence of AZM5_{—}23292 is shown in SEQ ID NO: 77.

### Isolation of the Promoter Region by PCR Amplification

The putative promoter region was isolated via genomic PCR using the following sequence specific primers:

Forward primer:

(SEQ ID NO: 186)

GTGATTAAGTTGACTGGCAAATTG
Reverse primer:

(SEQ ID NO: 187)

GCCTACTTGCCTAGCGTACC.

The expected 698 bp fragment was amplified from maize genomic DNA, and named as promoter MAEM20 (p-MAEM20). Sequence of p-MAEM20 was shown in SEQ ID NO: 5.
BLASTN Results of p_MAEM20

The top 16 homologous sequences identified in the BlastN query of p_MAEM20 are presented in Table 48.

TABLE 48
BLASTN results of p_MAEM20

| | | Max | Total | Query | | Max |
|---|---|---|---|---|---|---|
| Accession | Description | score | score | coverage | E value | ident |
| EU951788.1 | Zea mays clone | 196 | 196 | 15% | 2.00E−46 | 100% |
| | 1000340 unknown | | | | | |
| | mRNA | | | | | |
| CP000820.1 | Frankia sp. | 44.6 | 44.6 | 5% | 0.64 | 86% |
| | EAN1pec, | | | | | |
| | complete genome | | | | | |
| AC174361.12 | Medicago | 42.8 | 42.8 | 4% | 2.2 | 93% |
| | truncatula | | | | | |
| | chromosome 8 | | | | | |
| | clone mth2-39o9, | | | | | |
| | complete sequence | | | | | |
| AC146791.12 | Medicago | 42.8 | 42.8 | 4% | 2.2 | 93% |
| | truncatula | | | | | |
| | chromosome 8 | | | | | |
| | clone mth2- | | | | | |
| | 123m17, complete | | | | | |
| | sequence | | | | | |
| XM_001502388.2 | PREDICTED: | 41 | 41 | 3% | 7.8 | 92% |
| | Equus caballus | | | | | |
| | similar to | | | | | |
| | neurotrypsin | | | | | |
| | (LOC100072455), | | | | | |
| | mRNA | | | | | |
| CP000548.1 | Burkholderia mallei | 41 | 41 | 3% | 7.8 | 92% |
| | NCTC 10247 | | | | | |
| | chromosome I, | | | | | |
| | complete sequence | | | | | |
| CP000572.1 | Burkholderia | 41 | 41 | 3% | 7.8 | 92% |
| | pseudomallei | | | | | |
| | 1106a | | | | | |
| | chromosome I, | | | | | |
| | complete sequence | | | | | |
| CP000546.1 | Burkholderia mallei | 41 | 41 | 3% | 7.8 | 92% |
| | NCTC 10229 | | | | | |
| | chromosome I, | | | | | |
| | complete sequence | | | | | |
| CP000526.1 | Burkholderia mallei | 41 | 41 | 3% | 7.8 | 92% |
| | SAVP1 | | | | | |
| | chromosome I, | | | | | |
| | complete sequence | | | | | |
| CP000539.1 | Acidovorax sp. | 41 | 41 | 5% | 7.8 | 85% |
| | JS42, complete | | | | | |
| | genome | | | | | |
| CP000489.1 | Paracoccus | 41 | 41 | 4% | 7.8 | 90% |
| | denitrificans | | | | | |
| | PD1222 | | | | | |
| | chromosome 1, | | | | | |
| | complete sequence | | | | | |
| EF130439.1 | Sus scrofa clone | 41 | 41 | 5% | 7.8 | 86% |
| | KVL4379 | | | | | |
| | microsatellite | | | | | |
| | sequence | | | | | |
| CP000383.1 | Cytophaga | 41 | 41 | 3% | 7.8 | 96% |
| | hutchinsonii ATCC | | | | | |
| | 33406, complete | | | | | |
| | genome | | | | | |
| XM_001106371.1 | PREDICTED: | 41 | 41 | 4% | 7.8 | 90% |
| | Macaca mulatta | | | | | |
| | similar to | | | | | |
| | chromosome 9 | | | | | |
| | open reading frame | | | | | |
| | 58 isoform 1, | | | | | |
| | transcript variant 4 | | | | | |
| | (LOC715655), | | | | | |
| | mRNA | | | | | |
| CP000010.1 | Burkholderia mallei | 41 | 41 | 3% | 7.8 | 92% |
| | ATCC 23344 | | | | | |
| | chromosome 1, | | | | | |
| | complete sequence | | | | | |

### Example 11

### Place Analysis of the Promoters

Cis-acting motifs in the promoter regions were identified using PLACE (a database of Plant Cis-acting Regulatory DNA elements) using the Genomatix database suite.

1) p-MAWS23

PLACE analysis results of p-MAWS23 are listed in Table 49. Two TATA box motifs are found in this promoter, one located at nucleotide position 419-425 of the forward strand, the other located at nucleotide position 736-742 of the reverse strand. There is 1 CAAT Box motif at nucleotide position 621-625 of the forward strand.

TABLE 49
PLACE analysis results of the 1264 bp promoter p-MAWS23

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM324 | CGCGBOXAT | 23 | 28 | + | 0 | 1 | GCGCGT |
| FAM324 | CGCGBOXAT | 23 | 28 | − | 0 | 1 | ACGCGC |
| FAM107 | CGACGOSAMY3 | 26 | 30 | − | 0 | 1 | CGACG |
| FAM261 | CDTDREHVCBF2 | 27 | 32 | + | 0 | 1 | GTCGAC |
| FAM261 | CDTDREHVCBF2 | 27 | 32 | − | 0 | 1 | GTCGAC |
| FAM107 | CGACGOSAMY3 | 29 | 33 | + | 0 | 1 | CGACG |
| FAM002 | TGACGTVMAMY | 48 | 60 | + | 0 | 1 | GACTATGACGTCA |
| FAM002 | PALINDROMICCBOXGM | 50 | 62 | − | 0 | 1 | GATGACGTCATAG |
| FAM002 | PALINDROMICCBOXGM | 51 | 63 | + | 0 | 1 | TATGACGTCATCT |
| FAM002 | TGACGTVMAMY | 53 | 65 | − | 0 | 1 | CAAGATGACGTCA |
| FAM057 | ACGTCBOX | 54 | 59 | + | 0 | 1 | GACGTC |
| FAM057 | ACGTCBOX | 54 | 59 | − | 0 | 1 | GACGTC |
| FAM010 | WBOXATNPR1 | 62 | 76 | + | 0 | 1 | CTTGACACCAGAGGT |
| FAM322 | BIHD1OS | 64 | 68 | − | 0 | 1 | TGTCA |
| FAM263 | DPBFCOREDCDC3 | 66 | 72 | + | 0 | 1 | ACACCAG |
| FAM322 | BIHD1OS | 76 | 80 | − | 0 | 1 | TGTCA |
| FAM270 | RAV1AAT | 92 | 96 | + | 0 | 1 | CAACA |
| FAM270 | RAV1AAT | 95 | 99 | + | 0 | 1 | CAACA |
| FAM266 | MYB1AT | 101 | 106 | + | 0 | 1 | TAACCA |
| FAM002 | SORLIP1AT | 104 | 116 | + | 0 | 1 | CCACTCGCCACCG |
| FAM147 | HEXAMERATH4 | 114 | 119 | + | 0 | 1 | CCGTCG |
| FAM013 | DRE2COREZMRAB17 | 115 | 121 | − | 0 | 1 | ACCGACG |
| FAM107 | CGACGOSAMY3 | 115 | 119 | − | 0 | 1 | CGACG |
| FAM013 | DRE2COREZMRAB17 | 119 | 125 | − | 0 | 1 | ACCGACC |
| FAM267 | TAAAGSTKST1 | 130 | 136 | − | 0 | 1 | ATTAAAG |
| FAM304 | OSE2ROOTNODULE | 138 | 142 | + | 0 | 1 | CTCTT |
| FAM024 | 2SSEEDPROTBANAPA | 145 | 153 | + | 0 | 1 | CAAACACAT |
| FAM263 | DPBFCOREDCDC3 | 154 | 160 | + | 0 | 1 | ACACTTG |
| FAM305 | ANAERO1CONSENSUS | 165 | 171 | − | 0 | 1 | AAACAAA |
| FAM314 | SORLIP4AT | 182 | 190 | + | 0 | 1 | GTATGATGG |
| FAM010 | WBOXHVISO1 | 200 | 214 | + | 0 | 1 | GATGACTGACAATGT |
| FAM322 | BIHD1OS | 206 | 210 | − | 0 | 1 | TGTCA |
| FAM002 | RAV1BAT | 230 | 242 | − | 0 | 1 | TTACACCTGCCGG |
| FAM263 | DPBFCOREDCDC3 | 234 | 240 | − | 0 | 1 | ACACCTG |
| FAM002 | CACGTGMOTIF | 239 | 251 | − | 0 | 1 | GAGCACGTGTTAC |
| FAM002 | CACGTGMOTIF | 240 | 252 | + | 0 | 1 | TAACACGTGCTCT |
| FAM263 | DPBFCOREDCDC3 | 242 | 248 | + | 0 | 1 | ACACGTG |
| FAM304 | OSE2ROOTNODULE | 249 | 253 | + | 0 | 1 | CTCTT |
| FAM012 | IBOXCORENT | 258 | 264 | + | 0 | 1 | GATAAGA |
| FAM026 | RYREPEATBNNAPA | 264 | 274 | + | 0 | 1 | ATCATGCAAAT |
| FAM311 | EECCRCAH1 | 269 | 275 | − | 0 | 1 | GATTTGC |
| FAM322 | BIHD1OS | 277 | 281 | + | 0 | 1 | TGTCA |
| FAM026 | RYREPEATBNNAPA | 291 | 301 | − | 0 | 1 | ATCATGCAGGC |
| FAM151 | INTRONLOWER | 292 | 297 | − | 0 | 1 | TGCAGG |
| FAM170 | MYBGAHV | 343 | 349 | + | 0 | 1 | TAACAAA |
| FAM300 | LECPLEACS2 | 382 | 389 | + | 0 | 1 | TAAAATAT |
| FAM243 | TATABOX4 | 419 | 425 | + | 0 | 1 | TATATAA |
| FAM270 | RAV1AAT | 449 | 453 | − | 0 | 1 | CAACA |
| FAM295 | P1BS | 475 | 482 | + | 0 | 1 | GTATATCC |
| FAM295 | P1BS | 475 | 482 | − | 0 | 1 | GGATATAC |
| FAM014 | MYBST1 | 477 | 483 | − | 0 | 1 | TGGATAT |
| FAM025 | AMYBOX2 | 478 | 484 | + | 0 | 1 | TATCCAT |
| FAM273 | TATCCAOSAMY | 478 | 484 | + | 0 | 1 | TATCCAT |
| FAM107 | CGACGOSAMY3 | 502 | 506 | − | 0 | 1 | CGACG |
| FAM026 | RYREPEATBNNAPA | 514 | 524 | − | 0 | 1 | CTCATGCAAGC |
| FAM261 | CDTDREHVCBF2 | 526 | 531 | + | 0 | 1 | GTCGAC |
| FAM261 | CDTDREHVCBF2 | 526 | 531 | − | 0 | 1 | GTCGAC |
| FAM304 | OSE2ROOTNODULE | 532 | 536 | + | 0 | 1 | CTCTT |
| FAM304 | OSE2ROOTNODULE | 537 | 541 | + | 0 | 1 | CTCTT |
| FAM012 | IBOXCORE | 540 | 546 | − | 0 | 1 | GATAAAA |
| FAM014 | SREATMSD | 541 | 547 | + | 0 | 1 | TTTATCC |
| FAM014 | MYBST1 | 542 | 548 | − | 0 | 1 | GGGATAA |
| FAM006 | HDZIP2ATATHB2 | 588 | 596 | − | 0 | 1 | TAATAATTA |
| FAM015 | ACGTABOX | 597 | 602 | + | 0 | 1 | TACGTA |
| FAM015 | ACGTABOX | 597 | 602 | − | 0 | 1 | TACGTA |
| FAM010 | WBOXHVISO1 | 600 | 614 | − | 0 | 1 | TATGACTTGAAGTAC |
| FAM266 | MYB1AT | 618 | 623 | + | 0 | 1 | AAACCA |
| FAM003 | REALPHALGLHCB21 | 619 | 629 | + | 0 | 1 | AACCAATGGCA |
| FAM100 | CCAATBOX1 | 621 | 625 | + | 0 | 1 | CCAAT |
| FAM266 | MYB1AT | 640 | 645 | + | 0 | 1 | TAACCA |
| FAM267 | TAAAGSTKST1 | 668 | 674 | + | 0 | 1 | AATAAAG |
| FAM244 | TATABOXOSPAL | 736 | 742 | − | 0 | 1 | TATTTAA |
| FAM267 | TAAAGSTKST1 | 756 | 762 | − | 0 | 1 | AATAAAG |
| FAM171 | MYBPZM | 782 | 788 | − | 0 | 1 | TCCAACC |
| FAM061 | GCCCORE | 795 | 801 | − | 0 | 1 | CGCCGCC |
| FAM026 | RYREPEATVFLEB4 | 812 | 822 | + | 0 | 1 | ACCATGCATGT |
| FAM026 | RYREPEATVFLEB4 | 813 | 823 | − | 0 | 1 | CACATGCATGG |
| FAM172 | MYCATRD2 | 817 | 823 | − | 0 | 1 | CACATGC |
| FAM172 | MYCATERD | 818 | 824 | + | 0 | 1 | CATGTGT |
| FAM263 | DPBFCOREDCDC3 | 818 | 824 | − | 0 | 1 | ACACATG |
| FAM012 | IBOXCORENT | 843 | 849 | − | 0 | 1 | GATAAGA |
| FAM171 | MYBPZM | 854 | 860 | + | 0 | 1 | TCCTACC |
| FAM304 | OSE2ROOTNODULE | 864 | 868 | + | 0 | 1 | CTCTT |
| FAM267 | TAAAGSTKST1 | 866 | 872 | − | 0 | 1 | ATTAAAG |
| FAM010 | WBBOXPCWRKY1 | 872 | 886 | + | 0 | 1 | TTTGACTCTTTATGA |
| FAM304 | OSE2ROOTNODULE | 877 | 881 | + | 0 | 1 | CTCTT |
| FAM267 | TAAAGSTKST1 | 879 | 885 | − | 0 | 1 | CATAAAG |
| FAM311 | EECCRCAH1 | 889 | 895 | − | 0 | 1 | GAATTCC |
| FAM311 | EECCRCAH1 | 890 | 896 | + | 0 | 1 | GAATTCC |
| FAM098 | CATATGGMSAUR | 897 | 902 | + | 0 | 1 | CATATG |
| FAM098 | CATATGGMSAUR | 897 | 902 | − | 0 | 1 | CATATG |
| FAM087 | BOXIINTPATPB | 905 | 910 | − | 0 | 1 | ATAGAA |
| FAM270 | RAV1AAT | 915 | 919 | + | 0 | 1 | CAACA |
| FAM107 | CGACGOSAMY3 | 923 | 927 | + | 0 | 1 | CGACG |
| FAM057 | ACGTCBOX | 924 | 929 | + | 0 | 1 | GACGTC |
| FAM057 | ACGTCBOX | 924 | 929 | − | 0 | 1 | GACGTC |
| FAM013 | DRECRTCOREAT | 957 | 963 | + | 0 | 1 | GCCGACG |
| FAM107 | CGACGOSAMY3 | 959 | 963 | + | 0 | 1 | CGACG |
| FAM147 | HEXAMERATH4 | 959 | 964 | − | 0 | 1 | CCGTCG |
| FAM026 | SPHCOREZMC1 | 986 | 996 | + | 0 | 1 | TCCATGCATGC |
| FAM026 | RYREPEATVFLEB4 | 987 | 997 | − | 0 | 1 | TGCATGCATGG |
| FAM026 | RYREPEATBNNAPA | 990 | 1000 | + | 0 | 1 | TGCATGCAAAT |
| FAM172 | MYCATERD | 1005 | 1011 | − | 0 | 1 | CATGTGT |
| FAM263 | DPBFCOREDCDC3 | 1005 | 1011 | + | 0 | 1 | ACACATG |
| FAM172 | MYCATRD2 | 1006 | 1012 | + | 0 | 1 | CACATGT |
| FAM205 | PYRIMIDINEBOXOSRAM | 1018 | 1023 | + | 0 | 1 | CCTTTT |
| FAM026 | RYREPEATLEGUMINBOX | 1030 | 1040 | + | 0 | 1 | GGCATGCACCC |
| FAM002 | SORLIP1AT | 1059 | 1071 | − | 0 | 1 | TTCACGGCCACGG |
| FAM322 | BIHD1OS | 1074 | 1078 | − | 0 | 1 | TGTCA |
| FAM324 | CGCGBOXAT | 1100 | 1105 | + | 0 | 1 | CCGCGC |
| FAM324 | CGCGBOXAT | 1100 | 1105 | − | 0 | 1 | GCGCGG |
| FAM324 | CGCGBOXAT | 1102 | 1107 | + | 0 | 1 | GCGCGT |
| FAM324 | CGCGBOXAT | 1102 | 1107 | − | 0 | 1 | ACGCGC |
| FAM322 | BIHD1OS | 1112 | 1116 | + | 0 | 1 | TGTCA |
| FAM026 | RYREPEATBNNAPA | 1137 | 1147 | + | 0 | 1 | TCCATGCAAGC |
| FAM002 | SORLIP1AT | 1152 | 1164 | + | 0 | 1 | ACCCGGGCCACGT |
| FAM302 | SORLIP2AT | 1155 | 1165 | + | 0 | 1 | CGGGCCACGTA |
| FAM002 | ABREATCONSENSUS | 1156 | 1168 | − | 0 | 1 | GGGTACGTGGCCC |
| FAM002 | ABREMOTIFAOSOSEM | 1179 | 1191 | + | 0 | 1 | CGCTACGTGTCAC |
| FAM322 | BIHD1OS | 1186 | 1190 | + | 0 | 1 | TGTCA |
| FAM002 | ASF1MOTIFCAMV | 1195 | 1207 | − | 0 | 1 | ATAGGTGACGAGA |
| FAM272 | SV40COREENHAN | 1228 | 1235 | − | 0 | 1 | GTGGAAAG |
| FAM002 | ASF1MOTIFCAMV | 1243 | 1255 | − | 0 | 1 | CAAAGTGACGGAG |
| FAM245 | TBOXATGAPB | 1250 | 1255 | + | 0 | 1 | ACTTTG |
| FAM305 | ANAERO1CONSENSUS | 1252 | 1258 | − | 0 | 1 | AAACAAA |

2) p-MAWS27

PLACE analysis results of p-MAWS27 are listed in Table 50. Multiple TATA box motifs are found in this promoter, located at nucleotide position 1-7, 278-284, 597-603, 1246-1252 of the forward strand, and 273-279, 533-539 of the reverse strand, respectively. Three CAAT Box motifs are located at nucleotide position −947-951, 968-972 and 985-989 of the forward strand.

TABLE 50
PLACE analysis results of the 1355 bp promoter p-MAWS27

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM241 | TATABOX2 | 1 | 7 | + | 0 | 1 | TATAAAT |
| FAM325 | MYBCOREATCYCB1 | 11 | 15 | + | 0 | 1 | AACGG |
| FAM002 | T/GBOXATPIN2 | 54 | 66 | − | 0 | 1 | TTAAACGTGATGA |
| FAM307 | ANAERO3CONSENSUS | 54 | 60 | + | 0 | 1 | TCATCAC |
| FAM305 | ANAERO1CONSENSUS | 67 | 73 | − | 0 | 1 | AAACAAA |
| FAM292 | PREATPRODH | 84 | 89 | − | 0 | 1 | ACTCAT |
| FAM008 | MYB2AT | 90 | 100 | − | 0 | 1 | GCTGTAACTGA |
| FAM012 | IBOXCORE | 105 | 111 | + | 0 | 1 | GATAATT |
| FAM107 | CGACGOSAMY3 | 112 | 116 | + | 0 | 1 | CGACG |
| FAM147 | HEXAMERATH4 | 112 | 117 | − | 0 | 1 | CCGTCG |
| FAM026 | RYREPEATBNNAPA | 123 | 133 | − | 0 | 1 | TGCATGCAAAT |
| FAM026 | RYREPEATLEGUMINBOX | 126 | 136 | + | 0 | 1 | TGCATGCACTT |
| FAM290 | GT1GMSCAM4 | 207 | 212 | − | 0 | 1 | GAAAAA |
| FAM304 | OSE2ROOTNODULE | 212 | 216 | + | 0 | 1 | CTCTT |
| FAM014 | REBETALGLHCB21 | 217 | 223 | + | 0 | 1 | CGGATAC |
| FAM014 | REBETALGLHCB21 | 223 | 229 | + | 0 | 1 | CGGATAT |
| FAM012 | IBOXCORE | 227 | 233 | − | 0 | 1 | GATAATA |
| FAM260 | CAREOSREP1 | 234 | 239 | − | 0 | 1 | CAACTC |
| FAM244 | TATABOXOSPAL | 273 | 279 | − | 0 | 1 | TATTTAA |
| FAM243 | TATABOX4 | 278 | 284 | + | 0 | 1 | TATATAA |
| FAM245 | TBOXATGAPB | 284 | 289 | + | 0 | 1 | ACTTTG |
| FAM295 | P1BS | 289 | 296 | + | 0 | 1 | GAATATAC |
| FAM295 | P1BS | 289 | 296 | − | 0 | 1 | GTATATTC |
| FAM014 | REBETALGLHCB21 | 296 | 302 | + | 0 | 1 | CGGATAC |
| FAM015 | ACGTABOX | 300 | 305 | + | 0 | 1 | TACGTA |
| FAM015 | ACGTABOX | 300 | 305 | − | 0 | 1 | TACGTA |
| FAM014 | REBETALGLHCB21 | 313 | 319 | + | 0 | 1 | CGGATAT |
| FAM262 | CIACADIANLELHC | 340 | 349 | − | 0 | 1 | CAATTTAATC |
| FAM100 | CCAATBOX1 | 346 | 350 | − | 0 | 1 | CCAAT |
| FAM280 | AGMOTIFNTMYB2 | 347 | 354 | − | 0 | 1 | AGATCCAA |
| FAM024 | PROXBBNNAPA | 368 | 376 | − | 0 | 1 | CAAACACCC |
| FAM310 | CPBCSPOR | 389 | 394 | − | 0 | 1 | TATTAG |
| FAM002 | SORLIP1AT | 397 | 409 | − | 0 | 1 | ATTTTAGCCACTA |
| FAM024 | PROXBBNNAPA | 423 | 431 | + | 0 | 1 | CAAACACCC |
| FAM310 | CPBCSPOR | 435 | 440 | − | 0 | 1 | TATTAG |
| FAM310 | CPBCSPOR | 448 | 453 | + | 0 | 1 | TATTAG |
| FAM012 | IBOXCORE | 485 | 491 | − | 0 | 1 | GATAACT |
| FAM170 | AMYBOX1 | 490 | 496 | − | 0 | 1 | TAACAGA |
| FAM156 | L1BOXATPDF1 | 528 | 535 | − | 0 | 1 | TAAATGCA |
| FAM244 | TATABOXOSPAL | 533 | 539 | − | 0 | 1 | TATTTAA |
| FAM025 | AMYBOX2 | 569 | 575 | − | 0 | 1 | TATCCAT |
| FAM273 | TATCCAOSAMY | 569 | 575 | − | 0 | 1 | TATCCAT |
| FAM014 | MYBST1 | 570 | 576 | + | 0 | 1 | TGGATAA |
| FAM014 | SREATMSD | 571 | 577 | − | 0 | 1 | ATTATCC |
| FAM012 | IBOXCORE | 572 | 578 | + | 0 | 1 | GATAATA |
| FAM014 | MYBST1 | 576 | 582 | − | 0 | 1 | TGGATAT |
| FAM025 | AMYBOX2 | 577 | 583 | + | 0 | 1 | TATCCAT |
| FAM273 | TATCCAOSAMY | 577 | 583 | + | 0 | 1 | TATCCAT |
| FAM202 | -300ELEMENT | 586 | 594 | − | 0 | 1 | TGTAAAATG |
| FAM227 | SEF1MOTIF | 597 | 605 | − | 0 | 1 | ATATTTATA |
| FAM241 | TATABOX2 | 597 | 603 | + | 0 | 1 | TATAAAT |
| FAM010 | WBOXATNPR1 | 616 | 630 | − | 0 | 1 | TTTGACATCTATATA |
| FAM322 | BIHD1OS | 624 | 628 | + | 0 | 1 | TGTCA |
| FAM171 | MYBPZM | 635 | 641 | − | 0 | 1 | CCCAACC |
| FAM270 | RAV1AAT | 644 | 648 | − | 0 | 1 | CAACA |
| FAM002 | SORLIP1AT | 655 | 667 | − | 0 | 1 | TATCGTGCCACGG |
| FAM276 | TRANSINITDICOTS | 686 | 693 | + | 0 | 1 | AACATGGC |
| FAM061 | GCCCORE | 696 | 702 | − | 0 | 1 | CGCCGCC |
| FAM290 | GT1GMSCAM4 | 742 | 747 | − | 0 | 1 | GAAAAA |
| FAM260 | CAREOSREP1 | 808 | 813 | + | 0 | 1 | CAACTC |
| FAM012 | IBOX | 863 | 869 | − | 0 | 1 | GATAAGC |
| FAM303 | OSE1ROOTNODULE | 866 | 872 | − | 0 | 1 | AAAGATA |
| FAM228 | SEF3MOTIFGM | 909 | 914 | + | 0 | 1 | AACCCA |
| FAM205 | PYRIMIDINEBOXOSRAM | 940 | 945 | + | 0 | 1 | CCTTTT |
| FAM100 | CCAATBOX1 | 947 | 951 | + | 0 | 1 | CCAAT |
| FAM002 | ABRELATERD | 954 | 966 | − | 0 | 1 | ACGGACGTGGTTT |
| FAM266 | MYB1AT | 954 | 959 | + | 0 | 1 | AAACCA |
| FAM194 | PALBOXAPC | 963 | 969 | + | 0 | 1 | CCGTCCC |
| FAM100 | CCAATBOX1 | 968 | 972 | + | 0 | 1 | CCAAT |
| FAM221 | S1FBOXSORPS1L21 | 971 | 976 | + | 0 | 1 | ATGGTA |
| FAM228 | SEF3MOTIFGM | 976 | 981 | + | 0 | 1 | AACCCA |
| FAM100 | CCAATBOX1 | 985 | 989 | + | 0 | 1 | CCAAT |
| FAM263 | DPBFCOREDCDC3 | 990 | 996 | − | 0 | 1 | ACACGAG |
| FAM024 | CANBNNAPA | 991 | 999 | − | 0 | 1 | CGAACACGA |
| FAM069 | ARFAT | 1003 | 1009 | − | 0 | 1 | CTGTCTC |
| FAM069 | SURECOREATSULTR11 | 1003 | 1009 | + | 0 | 1 | GAGACAG |
| FAM271 | SEBFCONSSTPR10A | 1003 | 1009 | − | 0 | 1 | CTGTCTC |
| FAM026 | RYREPEATBNNAPA | 1010 | 1020 | + | 0 | 1 | AGCATGCAAAC |
| FAM305 | ANAERO1CONSENSUS | 1017 | 1023 | + | 0 | 1 | AAACAAA |
| FAM039 | AACACOREOSGLUB1 | 1018 | 1024 | + | 0 | 1 | AACAAAC |
| FAM026 | RYREPEATVFLEB4 | 1025 | 1035 | + | 0 | 1 | AGCATGCATGC |
| FAM026 | RYREPEATVFLEB4 | 1026 | 1036 | − | 0 | 1 | CGCATGCATGC |
| FAM324 | CGCGBOXAT | 1053 | 1058 | + | 0 | 1 | GCGCGC |
| FAM324 | CGCGBOXAT | 1053 | 1058 | − | 0 | 1 | GCGCGC |
| FAM324 | CGCGBOXAT | 1055 | 1060 | + | 0 | 1 | GCGCGG |
| FAM324 | CGCGBOXAT | 1055 | 1060 | − | 0 | 1 | CCGCGC |
| FAM002 | ABRELATERD | 1058 | 1070 | + | 0 | 1 | CGGGACGTGAACC |
| FAM324 | CGCGBOXAT | 1069 | 1074 | + | 0 | 1 | CCGCGC |
| FAM324 | CGCGBOXAT | 1069 | 1074 | − | 0 | 1 | GCGCGG |
| FAM324 | CGCGBOXAT | 1071 | 1076 | + | 0 | 1 | GCGCGC |
| FAM324 | CGCGBOXAT | 1071 | 1076 | − | 0 | 1 | GCGCGC |
| FAM061 | GCCCORE | 1083 | 1089 | + | 0 | 1 | TGCCGCC |
| FAM194 | PALBOXAPC | 1089 | 1095 | + | 0 | 1 | CCGTCCG |
| FAM069 | SURECOREATSULTR11 | 1094 | 1100 | − | 0 | 1 | GAGACCG |
| FAM002 | ASF1MOTIFCAMV | 1139 | 1151 | + | 0 | 1 | CATCCTGACGCGC |
| FAM324 | CGCGBOXAT | 1146 | 1151 | + | 0 | 1 | ACGCGC |
| FAM324 | CGCGBOXAT | 1146 | 1151 | − | 0 | 1 | GCGCGT |
| FAM324 | CGCGBOXAT | 1148 | 1153 | + | 0 | 1 | GCGCGT |
| FAM324 | CGCGBOXAT | 1148 | 1153 | − | 0 | 1 | ACGCGC |
| FAM302 | SORLIP2AT | 1157 | 1167 | − | 0 | 1 | GGGGCCCAGAC |
| FAM302 | SITEIIATCYTC | 1160 | 1170 | + | 0 | 1 | TGGGCCCCAAA |
| FAM002 | ABRELATERD | 1169 | 1181 | − | 0 | 1 | GCGGACGTGGTTT |
| FAM266 | MYB1AT | 1169 | 1174 | + | 0 | 1 | AAACCA |
| FAM002 | ABREOSRAB21 | 1170 | 1182 | + | 0 | 1 | AACCACGTCCGCC |
| FAM324 | CGCGBOXAT | 1181 | 1186 | + | 0 | 1 | CCGCGG |
| FAM324 | CGCGBOXAT | 1181 | 1186 | − | 0 | 1 | CCGCGG |
| FAM061 | GCCCORE | 1185 | 1191 | − | 0 | 1 | CGCCGCC |
| FAM061 | GCCCORE | 1188 | 1194 | − | 0 | 1 | CGCCGCC |
| FAM324 | CGCGBOXAT | 1192 | 1197 | + | 0 | 1 | GCGCGC |
| FAM324 | CGCGBOXAT | 1192 | 1197 | − | 0 | 1 | GCGCGC |
| FAM061 | GCCCORE | 1236 | 1242 | + | 0 | 1 | TGCCGCC |
| FAM241 | TATABOX2 | 1246 | 1252 | + | 0 | 1 | TATAAAT |
| FAM156 | L1BOXATPDF1 | 1248 | 1255 | + | 0 | 1 | TAAATGCA |
| FAM069 | SURECOREATSULTR11 | 1269 | 1275 | − | 0 | 1 | GAGACGC |
| FAM246 | TCA1MOTIF | 1283 | 1292 | + | 0 | 1 | TCATCTTCTT |

3) p-MAWS30

PLACE analysis results of p-MAWS30 are listed in Table 51. One TATA box motif is found in this promoter, located at nucleotide position290-296 of the forward strand. No CAAT Box motifs are found in this promoter.

TABLE 51
PLACE analysis results of the 623 bp promoter p-MAWS30

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM234 | SP8BFIBSP8BIB | 15 | 21 | − | 0 | 1 | TACTATT |
| FAM267 | TAAAGSTKST1 | 18 | 24 | + | 0 | 1 | AGTAAAG |
| FAM267 | NTBBF1ARROLB | 19 | 25 | − | 0 | 1 | ACTTTAC |
| FAM266 | MYB1AT | 31 | 36 | − | 0 | 1 | TAACCA |
| FAM322 | BIHD1OS | 56 | 60 | + | 0 | 1 | TGTCA |
| FAM027 | -10PEHVPSBD | 72 | 77 | − | 0 | 1 | TATTCT |
| FAM010 | WBOXHVISO1 | 73 | 87 | − | 0 | 1 | CGTGACTACATATTC |
| FAM270 | RAV1AAT | 90 | 94 | + | 0 | 1 | CAACA |
| FAM290 | GT1GMSCAM4 | 123 | 128 | − | 0 | 1 | GAAAAA |
| FAM171 | MYBPZM | 132 | 138 | + | 0 | 1 | TCCAACC |
| FAM027 | -10PEHVPSBD | 152 | 157 | + | 0 | 1 | TATTCT |
| FAM156 | L1BOXATPDF1 | 167 | 174 | + | 0 | 1 | TAAATGTA |
| FAM014 | MYBST1 | 186 | 192 | − | 0 | 1 | AGGATAG |
| FAM205 | PYRIMIDINEBOXOSRAM | 190 | 195 | + | 0 | 1 | CCTTTT |
| FAM290 | GT1GMSCAM4 | 192 | 197 | − | 0 | 1 | GAAAAA |
| FAM008 | MYB2AT | 211 | 221 | + | 0 | 1 | GCATTAACTGA |
| FAM304 | OSE2ROOTNODULE | 255 | 259 | − | 0 | 1 | CTCTT |
| FAM162 | LTRE1HVBLT49 | 273 | 278 | + | 0 | 1 | CCGAAA |
| FAM170 | MYBGAHV | 280 | 286 | − | 0 | 1 | TAACAAA |
| FAM241 | TATABOX2 | 290 | 296 | + | 0 | 1 | TATAAAT |
| FAM066 | AMMORESIVDCRNIA1 | 313 | 319 | − | 0 | 1 | CGAACTT |
| FAM266 | MYB1AT | 343 | 348 | + | 0 | 1 | TAACCA |
| FAM010 | WBOXNTCHN48 | 358 | 372 | + | 0 | 1 | GCTGACTCGACCACC |
| FAM026 | RYREPEATLEGUMINBOX | 391 | 401 | + | 0 | 1 | TCCATGCACAT |
| FAM172 | MYCATERD | 396 | 402 | − | 0 | 1 | CATGTGC |
| FAM172 | MYCATRD2 | 397 | 403 | + | 0 | 1 | CACATGT |
| FAM010 | WBOXHVISO1 | 443 | 457 | + | 0 | 1 | CATGACTCTGACAGC |
| FAM322 | BIHD1OS | 451 | 455 | − | 0 | 1 | TGTCA |
| FAM322 | BIHD1OS | 482 | 486 | + | 0 | 1 | TGTCA |
| FAM315 | SORLIP5AT | 489 | 495 | − | 0 | 1 | GAGTGAG |
| FAM026 | RYREPEATBNNAPA | 507 | 517 | + | 0 | 1 | TCCATGCAAGC |
| FAM002 | SORLIP1AT | 522 | 534 | + | 0 | 1 | ACCTCGGCCACGT |
| FAM002 | ABREATCONSENSUS | 526 | 538 | − | 0 | 1 | GGGTACGTGGCCG |
| FAM002 | ABREMOTIFAOSOSEM | 548 | 560 | + | 0 | 1 | CCTTACGTGTCAC |
| FAM322 | BIHD1OS | 555 | 559 | + | 0 | 1 | TGTCA |
| FAM272 | SV40COREENHAN | 597 | 604 | − | 0 | 1 | GTGGAAAG |
| FAM294 | CTRMCAMV35S | 613 | 621 | + | 0 | 1 | TCTCTCTCT |
| FAM294 | CTRMCAMV35S | 615 | 623 | + | 0 | 1 | TCTCTCTCT |

4) p-MAWS57

PLACE analysis results of p-MAWS57 are listed in Table 52. No TATA box motifs are found in this promoter. Four CAAT box motifs are located at nucleotide position 217-221, 423-427, 501-505 of the forward strand and 340-344 of the reverse strand, respectively.

TABLE 52
PLACE analysis results of the 1950 bp promoter p-MAWS57

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM307 | ANAERO3CONSENSUS | 17 | 23 | − | 0 | 1 | TCATCAC |
| FAM266 | MYB1AT | 46 | 51 | − | 0 | 1 | AAACCA |
| FAM304 | OSE2ROOTNODULE | 118 | 122 | − | 0 | 1 | CTCTT |
| FAM069 | ARFAT | 120 | 126 | − | 0 | 1 | ATGTCTC |
| FAM069 | SURECOREATSULTR11 | 120 | 126 | + | 0 | 1 | GAGACAT |
| FAM010 | WBOXATNPR1 | 159 | 173 | + | 0 | 1 | GTTGACTGGTTGTCT |
| FAM100 | CCAATBOX1 | 217 | 221 | + | 0 | 1 | CCAAT |
| FAM172 | MYCATRD2 | 230 | 236 | − | 0 | 1 | CACATGT |
| FAM172 | MYCATERD | 231 | 237 | + | 0 | 1 | CATGTGT |
| FAM263 | DPBFCOREDCDC3 | 231 | 237 | − | 0 | 1 | ACACATG |
| FAM026 | RYREPEATGMGY2 | 242 | 252 | + | 0 | 1 | AACATGCATTT |
| FAM026 | RYREPEATBNNAPA | 318 | 328 | − | 0 | 1 | AACATGCAAAT |
| FAM270 | RAV1AAT | 325 | 329 | − | 0 | 1 | CAACA |
| FAM273 | TATCCAOSAMY | 327 | 333 | − | 0 | 1 | TATCCAA |
| FAM014 | MYBST1 | 328 | 334 | + | 0 | 1 | TGGATAT |
| FAM100 | CCAATBOX1 | 340 | 344 | − | 0 | 1 | CCAAT |
| FAM304 | OSE2ROOTNODULE | 374 | 378 | − | 0 | 1 | CTCTT |
| FAM302 | SITEIIATCYTC | 415 | 425 | − | 0 | 1 | TGGGCTCTTTC |
| FAM304 | OSE2ROOTNODULE | 417 | 421 | − | 0 | 1 | CTCTT |
| FAM100 | CCAATBOX1 | 423 | 427 | + | 0 | 1 | CCAAT |
| FAM002 | SORLIP1AT | 448 | 460 | − | 0 | 1 | CACCCTGCCACCC |
| FAM304 | OSE2ROOTNODULE | 468 | 472 | + | 0 | 1 | CTCTT |
| FAM100 | CCAATBOX1 | 501 | 505 | + | 0 | 1 | CCAAT |
| FAM228 | SEF3MOTIFGM | 514 | 519 | + | 0 | 1 | AACCCA |
| FAM002 | RAV1BAT | 525 | 537 | − | 0 | 1 | TATCACCTGTGAA |
| FAM304 | OSE2ROOTNODULE | 620 | 624 | − | 0 | 1 | CTCTT |
| FAM292 | PREATPRODH | 637 | 642 | − | 0 | 1 | ACTCAT |
| FAM173 | NAPINMOTIFBN | 642 | 648 | + | 0 | 1 | TACACAT |
| FAM292 | PREATPRODH | 682 | 687 | − | 0 | 1 | ACTCAT |
| FAM260 | CAREOSREP1 | 684 | 689 | − | 0 | 1 | CAACTC |
| FAM263 | DPBFCOREDCDC3 | 691 | 697 | + | 0 | 1 | ACACAGG |
| FAM270 | RAV1AAT | 701 | 705 | + | 0 | 1 | CAACA |
| FAM273 | TATCCAOSAMY | 724 | 730 | − | 0 | 1 | TATCCAG |
| FAM014 | MYBST1 | 725 | 731 | + | 0 | 1 | TGGATAC |
| FAM304 | OSE2ROOTNODULE | 752 | 756 | − | 0 | 1 | CTCTT |
| FAM263 | DPBFCOREDCDC3 | 768 | 774 | − | 0 | 1 | ACACTGG |
| FAM010 | WBOXATNPR1 | 769 | 783 | − | 0 | 1 | CTTGACACCACACTG |
| FAM322 | BIHD1OS | 777 | 781 | + | 0 | 1 | TGTCA |
| FAM295 | P1BS | 790 | 797 | + | 0 | 1 | GTATATGC |
| FAM295 | P1BS | 790 | 797 | − | 0 | 1 | GCATATAC |
| FAM304 | OSE2ROOTNODULE | 799 | 803 | − | 0 | 1 | CTCTT |
| FAM010 | WBOXHVISO1 | 802 | 816 | − | 0 | 1 | GATGACTTGTATTCT |
| FAM027 | -10PEHVPSBD | 802 | 807 | − | 0 | 1 | TATTCT |
| FAM026 | RYREPEATGMGY2 | 866 | 876 | + | 0 | 1 | TTCATGCATAT |
| FAM263 | DPBFCOREDCDC3 | 890 | 896 | − | 0 | 1 | ACACTTG |
| FAM202 | -300ELEMENT | 899 | 907 | − | 0 | 1 | TGAAAAGGT |
| FAM205 | PYRIMIDINEBOXOSRAM | 900 | 905 | + | 0 | 1 | CCTTTT |
| FAM267 | TAAAGSTKST1 | 909 | 915 | + | 0 | 1 | TTTAAAG |
| FAM008 | MYB2AT | 916 | 926 | + | 0 | 1 | GCTGTAACTGT |
| FAM270 | RAV1AAT | 968 | 972 | − | 0 | 1 | CAACA |
| FAM295 | P1BS | 977 | 984 | + | 0 | 1 | GCATATAC |
| FAM295 | P1BS | 977 | 984 | − | 0 | 1 | GTATATGC |
| FAM310 | CPBCSPOR | 987 | 992 | − | 0 | 1 | TATTAG |
| FAM010 | WBOXATNPR1 | 988 | 1002 | − | 0 | 1 | TTTGACATTTTATTA |
| FAM322 | BIHD1OS | 996 | 1000 | + | 0 | 1 | TGTCA |
| FAM012 | IBOXCORE | 1035 | 1041 | + | 0 | 1 | GATAATT |
| FAM170 | MYBGAHV | 1064 | 1070 | − | 0 | 1 | TAACAAA |
| FAM290 | GT1GMSCAM4 | 1074 | 1079 | + | 0 | 1 | GAAAAA |
| FAM245 | TBOXATGAPB | 1086 | 1091 | + | 0 | 1 | ACTTTG |
| FAM321 | WRECSAA01 | 1105 | 1114 | − | 0 | 1 | AAAGTATCGA |
| FAM245 | TBOXATGAPB | 1110 | 1115 | + | 0 | 1 | ACTTTG |
| FAM010 | ELRECOREPCRP1 | 1121 | 1135 | + | 0 | 1 | ATTGACCCGTTACCA |
| FAM325 | MYBCOREATCYCB1 | 1127 | 1131 | − | 0 | 1 | AACGG |
| FAM008 | MYB2AT | 1133 | 1143 | − | 0 | 1 | GTGGTAACTGG |
| FAM010 | WBOXNTCHN48 | 1173 | 1187 | + | 0 | 1 | TCTGACTTGAAGAAG |
| FAM002 | RAV1BAT | 1205 | 1217 | + | 0 | 1 | GTCCACCTGAACG |
| FAM325 | MYBCOREATCYCB1 | 1214 | 1218 | + | 0 | 1 | AACGG |
| FAM069 | ARFAT | 1218 | 1224 | − | 0 | 1 | CTGTCTC |
| FAM069 | SURECOREATSULTR11 | 1218 | 1224 | + | 0 | 1 | GAGACAG |
| FAM271 | SEBFCONSSTPR10A | 1218 | 1224 | − | 0 | 1 | CTGTCTC |
| FAM002 | SORLIP1AT | 1231 | 1243 | − | 0 | 1 | CTCTCCGCCACAA |
| FAM002 | RAV1BAT | 1244 | 1256 | + | 0 | 1 | CTCCACCTGAACG |
| FAM171 | MYBPZM | 1283 | 1289 | − | 0 | 1 | GCCAACC |
| FAM002 | SORLIP1AT | 1289 | 1301 | − | 0 | 1 | CAGCTCGCCACGG |
| FAM002 | SORLIP1AT | 1296 | 1308 | + | 0 | 1 | GAGCTGGCCACCT |
| FAM069 | SURECOREATSULTR11 | 1311 | 1317 | − | 0 | 1 | GAGACTA |
| FAM324 | CGCGBOXAT | 1348 | 1353 | + | 0 | 1 | GCGCGG |
| FAM324 | CGCGBOXAT | 1348 | 1353 | − | 0 | 1 | CCGCGC |
| FAM263 | DPBFCOREDCDC3 | 1356 | 1362 | − | 0 | 1 | ACACTGG |
| FAM147 | HEXAMERATH4 | 1365 | 1370 | + | 0 | 1 | CCGTCG |
| FAM107 | CGACGOSAMY3 | 1366 | 1370 | − | 0 | 1 | CGACG |
| FAM228 | SEF3MOTIFGM | 1373 | 1378 | − | 0 | 1 | AACCCA |
| FAM061 | GCCCORE | 1387 | 1393 | + | 0 | 1 | GGCCGCC |
| FAM061 | GCCCORE | 1390 | 1396 | + | 0 | 1 | CGCCGCC |
| FAM209 | RBCSCONSENSUS | 1408 | 1414 | + | 0 | 1 | AATCCAA |
| FAM325 | MYBCOREATCYCB1 | 1414 | 1418 | + | 0 | 1 | AACGG |
| FAM302 | SITEIIATCYTC | 1455 | 1465 | + | 0 | 1 | TGGGCCTTATC |
| FAM012 | IBOXCORENT | 1459 | 1465 | − | 0 | 1 | GATAAGG |
| FAM002 | SORLIP1AT | 1463 | 1475 | + | 0 | 1 | ATCTAGGCCACAA |
| FAM059 | ACGTTBOX | 1474 | 1479 | + | 0 | 1 | AACGTT |
| FAM059 | ACGTTBOX | 1474 | 1479 | − | 0 | 1 | AACGTT |
| FAM010 | WBOXHVISO1 | 1480 | 1494 | + | 0 | 1 | TGTGACTCTGTGAGC |
| FAM302 | SITEIIATCYTC | 1500 | 1510 | − | 0 | 1 | TGGGCCCAAAC |
| FAM302 | SITEIIATCYTC | 1503 | 1513 | + | 0 | 1 | TGGGCCCATCT |
| FAM304 | OSE2ROOTNODULE | 1512 | 1516 | + | 0 | 1 | CTCTT |
| FAM012 | IBOXCORE | 1536 | 1542 | − | 0 | 1 | GATAAAA |
| FAM302 | SITEIIATCYTC | 1542 | 1552 | − | 0 | 1 | TGGGCTTGATG |
| FAM266 | MYB1AT | 1560 | 1565 | + | 0 | 1 | AAACCA |
| FAM171 | MYBPZM | 1569 | 1575 | + | 0 | 1 | TCCTACC |
| FAM305 | ANAERO1CONSENSUS | 1603 | 1609 | + | 0 | 1 | AAACAAA |
| FAM245 | TBOXATGAPB | 1606 | 1611 | − | 0 | 1 | ACTTTG |
| FAM172 | MYCATERD | 1628 | 1634 | − | 0 | 1 | CATGTGA |
| FAM172 | MYCATRD2 | 1629 | 1635 | + | 0 | 1 | CACATGC |
| FAM324 | CGCGBOXAT | 1634 | 1639 | + | 0 | 1 | GCGCGT |
| FAM324 | CGCGBOXAT | 1634 | 1639 | − | 0 | 1 | ACGCGC |
| FAM261 | CDTDREHVCBF2 | 1641 | 1646 | + | 0 | 1 | GTCGAC |
| FAM261 | CDTDREHVCBF2 | 1641 | 1646 | − | 0 | 1 | GTCGAC |
| FAM304 | OSE2ROOTNODULE | 1692 | 1696 | − | 0 | 1 | CTCTT |
| FAM276 | TRANSINITDICOTS | 1696 | 1703 | − | 0 | 1 | AACATGGC |
| FAM304 | OSE2ROOTNODULE | 1734 | 1738 | − | 0 | 1 | CTCTT |
| FAM324 | CGCGBOXAT | 1738 | 1743 | + | 0 | 1 | GCGCGT |
| FAM324 | CGCGBOXAT | 1738 | 1743 | − | 0 | 1 | ACGCGC |
| FAM061 | GCCCORE | 1765 | 1771 | + | 0 | 1 | GGCCGCC |
| FAM306 | ANAERO2CONSENSUS | 1772 | 1777 | + | 0 | 1 | AGCAGC |
| FAM324 | CGCGBOXAT | 1780 | 1785 | + | 0 | 1 | CCGCGG |
| FAM324 | CGCGBOXAT | 1780 | 1785 | − | 0 | 1 | CCGCGG |
| FAM002 | ABRELATERD | 1793 | 1805 | − | 0 | 1 | ACGGACGTGCTGC |
| FAM325 | MYBCOREATCYCB1 | 1802 | 1806 | − | 0 | 1 | AACGG |
| FAM302 | SORLIP2AT | 1806 | 1816 | − | 0 | 1 | CGGGCCGACCA |
| FAM013 | DRECRTCOREAT | 1807 | 1813 | − | 0 | 1 | GCCGACC |
| FAM002 | ABRELATERD | 1828 | 1840 | − | 0 | 1 | CGCGACGTGTGCC |
| FAM107 | CGACGOSAMY3 | 1834 | 1838 | − | 0 | 1 | CGACG |
| FAM026 | RYREPEATLEGUMINBOX | 1839 | 1849 | + | 0 | 1 | CGCATGCACGC |
| FAM324 | CGCGBOXAT | 1846 | 1851 | + | 0 | 1 | ACGCGC |
| FAM324 | CGCGBOXAT | 1846 | 1851 | − | 0 | 1 | GCGCGT |
| FAM324 | CGCGBOXAT | 1848 | 1853 | + | 0 | 1 | GCGCGG |
| FAM324 | CGCGBOXAT | 1848 | 1853 | − | 0 | 1 | CCGCGC |
| FAM002 | GADOWNAT | 1858 | 1870 | − | 0 | 1 | CGGCACGTGTCCG |
| FAM002 | CACGTGMOTIF | 1859 | 1871 | + | 0 | 1 | GGACACGTGCCGG |
| FAM263 | DPBFCOREDCDC3 | 1861 | 1867 | + | 0 | 1 | ACACGTG |
| FAM324 | CGCGBOXAT | 1871 | 1876 | + | 0 | 1 | GCGCGG |
| FAM324 | CGCGBOXAT | 1871 | 1876 | − | 0 | 1 | CCGCGC |
| FAM002 | ABREOSRAB21 | 1874 | 1886 | − | 0 | 1 | AGGGACGTGCCCG |
| FAM324 | CGCGBOXAT | 1889 | 1894 | + | 0 | 1 | CCGCGC |
| FAM324 | CGCGBOXAT | 1889 | 1894 | − | 0 | 1 | GCGCGG |
| FAM002 | SORLIP1AT | 1918 | 1930 | + | 0 | 1 | CCAGCAGCCACAA |
| FAM306 | ANAERO2CONSENSUS | 1920 | 1925 | + | 0 | 1 | AGCAGC |
| FAM270 | RAV1AAT | 1928 | 1932 | + | 0 | 1 | CAACA |

5) p-MAWS60

PLACE analysis results of p-MAWS60 are listed in Table 53. One TATA box motif is found at nucleotide position 156-162 of the forward strand. One CAAT box motif is located at nucleotide position 547-551 of the reverse strand.

TABLE 53
PLACE analysis results of the 1106 bp promoter p-MAWS60

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM266 | MYB1AT | 2 | 7 | − | 0 | 1 | AAACCA |
| FAM012 | IBOXCORE | 11 | 17 | + | 0 | 1 | GATAATT |
| FAM305 | ANAERO1CONSENSUS | 16 | 22 | − | 0 | 1 | AAACAAA |
| FAM012 | IBOXCORE | 19 | 25 | − | 0 | 1 | GATAAAC |
| FAM172 | MYCATERD | 26 | 32 | − | 0 | 1 | CATGTGA |
| FAM172 | MYCATRD2 | 27 | 33 | + | 0 | 1 | CACATGA |
| FAM290 | GT1GMSCAM4 | 77 | 82 | − | 0 | 1 | GAAAAA |
| FAM303 | OSE1ROOTNODULE | 95 | 101 | + | 0 | 1 | AAAGATA |
| FAM322 | BIHD1OS | 132 | 136 | + | 0 | 1 | TGTCA |
| FAM010 | ELRECOREPCRP1 | 140 | 154 | − | 0 | 1 | TTTGACCATTTCATT |
| FAM241 | TATABOX2 | 156 | 162 | + | 0 | 1 | TATAAAT |
| FAM292 | PREATPRODH | 174 | 179 | − | 0 | 1 | ACTCAT |
| FAM303 | OSE1ROOTNODULE | 200 | 206 | − | 0 | 1 | AAAGATT |
| FAM267 | TAAAGSTKST1 | 227 | 233 | − | 0 | 1 | TTTAAAG |
| FAM263 | DPBFCOREDCDC3 | 257 | 263 | − | 0 | 1 | ACACTAG |
| FAM270 | RAV1AAT | 261 | 265 | − | 0 | 1 | CAACA |
| FAM290 | GT1GMSCAM4 | 265 | 270 | + | 0 | 1 | GAAAAA |
| FAM311 | EECCRCAH1 | 285 | 291 | − | 0 | 1 | GAGTTTC |
| FAM304 | OSE2ROOTNODULE | 289 | 293 | + | 0 | 1 | CTCTT |
| FAM263 | DPBFCOREDCDC3 | 297 | 303 | − | 0 | 1 | ACACTCG |
| FAM263 | DPBFCOREDCDC3 | 311 | 317 | + | 0 | 1 | ACACTCG |
| FAM304 | OSE2ROOTNODULE | 327 | 331 | + | 0 | 1 | CTCTT |
| FAM010 | WBOXATNPR1 | 331 | 345 | − | 0 | 1 | TTTGACACTCGGCAA |
| FAM263 | DPBFCOREDCDC3 | 335 | 341 | − | 0 | 1 | ACACTCG |
| FAM322 | BIHD1OS | 339 | 343 | + | 0 | 1 | TGTCA |
| FAM300 | LECPLEACS2 | 348 | 355 | + | 0 | 1 | TAAAATAT |
| FAM267 | TAAAGSTKST1 | 400 | 406 | + | 0 | 1 | GGTAAAG |
| FAM263 | DPBFCOREDCDC3 | 418 | 424 | − | 0 | 1 | ACACTCG |
| FAM162 | LTRE1HVBLT49 | 425 | 430 | + | 0 | 1 | CCGAAA |
| FAM290 | GT1GMSCAM4 | 427 | 432 | + | 0 | 1 | GAAAAA |
| FAM012 | IBOXCORE | 439 | 445 | + | 0 | 1 | GATAAAA |
| FAM300 | LECPLEACS2 | 441 | 448 | + | 0 | 1 | TAAAATAT |
| FAM311 | EECCRCAH1 | 469 | 475 | + | 0 | 1 | GAATTCC |
| FAM010 | WBOXNTCHN48 | 477 | 491 | − | 0 | 1 | TCTGACTCACGCTAC |
| FAM022 | GCN4OSGLUB1 | 482 | 490 | + | 0 | 1 | GTGAGTCAG |
| FAM124 | ERELEE4 | 509 | 516 | − | 0 | 1 | ATTTCAAA |
| FAM262 | CIACADIANLELHC | 520 | 529 | + | 0 | 1 | CAAACAAATC |
| FAM305 | ANAERO1CONSENSUS | 521 | 527 | + | 0 | 1 | AAACAAA |
| FAM098 | CATATGGMSAUR | 540 | 545 | + | 0 | 1 | CATATG |
| FAM098 | CATATGGMSAUR | 540 | 545 | − | 0 | 1 | CATATG |
| FAM100 | CCAATBOX1 | 547 | 551 | − | 0 | 1 | CCAAT |
| FAM002 | SORLIP1AT | 550 | 562 | + | 0 | 1 | GGCTTTGCCACAT |
| FAM172 | MYCATERD | 557 | 563 | − | 0 | 1 | CATGTGG |
| FAM172 | MYCATRD2 | 558 | 564 | + | 0 | 1 | CACATGG |
| FAM221 | S1FBOXSORPS1L21 | 561 | 566 | + | 0 | 1 | ATGGTA |
| FAM263 | DPBFCOREDCDC3 | 578 | 584 | − | 0 | 1 | ACACTCG |
| FAM281 | MYB1LEPR | 603 | 609 | − | 0 | 1 | GTTAGTT |
| FAM228 | SEF3MOTIFGM | 607 | 612 | + | 0 | 1 | AACCCA |
| FAM266 | MYB1AT | 613 | 618 | − | 0 | 1 | AAACCA |
| FAM170 | AMYBOX1 | 622 | 628 | + | 0 | 1 | TAACAGA |
| FAM263 | DPBFCOREDCDC3 | 633 | 639 | + | 0 | 1 | ACACCAG |
| FAM010 | WBOXHVISO1 | 638 | 652 | + | 0 | 1 | AGTGACTCCATCGTT |
| FAM003 | MYB26PS | 739 | 749 | − | 0 | 1 | TGTTAGGTTGA |
| FAM003 | MYBPLANT | 741 | 751 | + | 0 | 1 | AACCTAACACA |
| FAM024 | CANBNNAPA | 744 | 752 | + | 0 | 1 | CTAACACAG |
| FAM026 | RYREPEATVFLEB4 | 762 | 772 | + | 0 | 1 | TACATGCATGC |
| FAM026 | RYREPEATVFLEB4 | 763 | 773 | − | 0 | 1 | CGCATGCATGT |
| FAM304 | OSE2ROOTNODULE | 789 | 793 | − | 0 | 1 | CTCTT |
| FAM002 | RAV1BAT | 794 | 806 | − | 0 | 1 | CATCACCTGCCTC |
| FAM307 | ANAERO3CONSENSUS | 801 | 807 | − | 0 | 1 | TCATCAC |
| FAM069 | SURECOREATSULTR11 | 811 | 817 | − | 0 | 1 | GAGACCT |
| FAM263 | DPBFCOREDCDC3 | 827 | 833 | − | 0 | 1 | ACACCAG |
| FAM002 | SORLIP1AT | 831 | 843 | + | 0 | 1 | TGTGCAGCCACGT |
| FAM002 | ABREATCONSENSUS | 835 | 847 | − | 0 | 1 | GGGTACGTGGCTG |
| FAM324 | CGCGBOXAT | 852 | 857 | + | 0 | 1 | ACGCGT |
| FAM324 | CGCGBOXAT | 852 | 857 | − | 0 | 1 | ACGCGT |
| FAM107 | CGACGOSAMY3 | 855 | 859 | − | 0 | 1 | CGACG |
| FAM324 | CGCGBOXAT | 860 | 865 | + | 0 | 1 | CCGCGG |
| FAM324 | CGCGBOXAT | 860 | 865 | − | 0 | 1 | CCGCGG |
| FAM107 | CGACGOSAMY3 | 867 | 871 | + | 0 | 1 | CGACG |
| FAM002 | RAV1BAT | 869 | 881 | − | 0 | 1 | CAACACCTGTCGT |
| FAM263 | DPBFCOREDCDC3 | 873 | 879 | − | 0 | 1 | ACACCTG |
| FAM024 | CANBNNAPA | 874 | 882 | − | 0 | 1 | CCAACACCT |
| FAM270 | RAV1AAT | 877 | 881 | − | 0 | 1 | CAACA |
| FAM171 | MYBPZM | 882 | 888 | + | 0 | 1 | GCCAACC |
| FAM322 | BIHD1OS | 912 | 916 | − | 0 | 1 | TGTCA |
| FAM015 | ACGTABOX | 932 | 937 | + | 0 | 1 | TACGTA |
| FAM015 | ACGTABOX | 932 | 937 | − | 0 | 1 | TACGTA |
| FAM069 | SURECOREATSULTR11 | 941 | 947 | + | 0 | 1 | GAGACGA |
| FAM024 | CANBNNAPA | 945 | 953 | + | 0 | 1 | CGAACACGA |
| FAM194 | PALBOXAPC | 963 | 969 | + | 0 | 1 | CCGTCCT |
| FAM002 | ASF1MOTIFCAMV | 978 | 990 | − | 0 | 1 | GAGCATGACGGGC |
| FAM026 | RYREPEATVFLEB4 | 988 | 998 | + | 0 | 1 | CTCATGCATGC |
| FAM026 | RYREPEATVFLEB4 | 989 | 999 | − | 0 | 1 | TGCATGCATGA |
| FAM026 | RYREPEATVFLEB4 | 992 | 1002 | + | 0 | 1 | TGCATGCATGC |
| FAM026 | RYREPEATVFLEB4 | 993 | 1003 | − | 0 | 1 | TGCATGCATGC |
| FAM026 | RYREPEATVFLEB4 | 996 | 1006 | + | 0 | 1 | TGCATGCATGC |
| FAM026 | RYREPEATVFLEB4 | 997 | 1007 | − | 0 | 1 | AGCATGCATGC |
| FAM026 | RYREPEATGMGY2 | 1009 | 1019 | + | 0 | 1 | ATCATGCATAC |
| FAM012 | IBOXCORE | 1022 | 1028 | + | 0 | 1 | GATAAAT |
| FAM015 | ACGTABOX | 1048 | 1053 | + | 0 | 1 | TACGTA |
| FAM015 | ACGTABOX | 1048 | 1053 | − | 0 | 1 | TACGTA |
| FAM151 | INTRONLOWER | 1092 | 1097 | + | 0 | 1 | TGCAGG |

6) p-MAWS63

PLACE analysis results of p-MAWS63 are listed in Table 54. Three TATA box motifs are found at nucleotide position 1555-1561, 1577-1583 and 1628-1634 of the forward strand, respectively. One CAAT box motif is located at nucleotide position 987-991 of the forward strand, and three CAAT box motifs are located at nucleotide position 156-160, 199-203, 249-253 of the reverse strand.

TABLE 54
PLACE analysis results of the 1941 bp promoter p-MAWS63

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM002 | SORLIP1AT | 8 | 20 | − | 0 | 1 | GTGTAGGCCACGA |
| FAM263 | DPBFCOREDCDC3 | 17 | 23 | + | 0 | 1 | ACACACG |
| FAM263 | DPBFCOREDCDC3 | 19 | 25 | + | 0 | 1 | ACACGCG |
| FAM324 | CGCGBOXAT | 21 | 26 | + | 0 | 1 | ACGCGC |
| FAM324 | CGCGBOXAT | 21 | 26 | − | 0 | 1 | GCGCGT |
| FAM008 | MYB2AT | 34 | 44 | − | 0 | 1 | ATGGTAACTGA |
| FAM221 | S1FBOXSORPS1L21 | 39 | 44 | − | 0 | 1 | ATGGTA |
| FAM173 | NAPINMOTIFBN | 73 | 79 | + | 0 | 1 | TACACAT |
| FAM172 | MYCATERD | 74 | 80 | − | 0 | 1 | CATGTGT |
| FAM263 | DPBFCOREDCDC3 | 74 | 80 | + | 0 | 1 | ACACATG |
| FAM172 | MYCATRD2 | 75 | 81 | + | 0 | 1 | CACATGA |
| FAM124 | ERELEE4 | 92 | 99 | − | 0 | 1 | AATTCAAA |
| FAM311 | EECCRCAH1 | 95 | 101 | + | 0 | 1 | GAATTTC |
| FAM026 | RYREPEATBNNAPA | 105 | 115 | + | 0 | 1 | AGCATGCAAAA |
| FAM202 | -300ELEMENT | 109 | 117 | + | 0 | 1 | TGCAAAATT |
| FAM012 | IBOXCORE | 136 | 142 | + | 0 | 1 | GATAAAA |
| FAM325 | MYBCOREATCYCB1 | 142 | 146 | + | 0 | 1 | AACGG |
| FAM002 | SORLIP1AT | 145 | 157 | − | 0 | 1 | ATTTTGGCCACCC |
| FAM100 | CCAATBOX1 | 156 | 160 | − | 0 | 1 | CCAAT |
| FAM061 | GCCCORE | 161 | 167 | − | 0 | 1 | TGCCGCC |
| FAM012 | IBOX | 177 | 183 | + | 0 | 1 | GATAAGC |
| FAM014 | MYBST1 | 184 | 190 | − | 0 | 1 | TGGATAG |
| FAM025 | AMYBOX2 | 185 | 191 | + | 0 | 1 | TATCCAT |
| FAM273 | TATCCAOSAMY | 185 | 191 | + | 0 | 1 | TATCCAT |
| FAM100 | CCAATBOX1 | 199 | 203 | − | 0 | 1 | CCAAT |
| FAM322 | BIHD1OS | 207 | 211 | + | 0 | 1 | TGTCA |
| FAM100 | CCAATBOX1 | 249 | 253 | − | 0 | 1 | CCAAT |
| FAM014 | MYBST1 | 258 | 264 | − | 0 | 1 | TGGATAG |
| FAM273 | TATCCAOSAMY | 259 | 265 | + | 0 | 1 | TATCCAG |
| FAM306 | ANAERO2CONSENSUS | 264 | 269 | + | 0 | 1 | AGCAGC |
| FAM008 | MYB2AT | 304 | 314 | + | 0 | 1 | TCCCTAACTGC |
| FAM002 | SORLIP1AT | 316 | 328 | + | 0 | 1 | CCGGCCGCCACAC |
| FAM061 | GCCCORE | 318 | 324 | + | 0 | 1 | GGCCGCC |
| FAM013 | LTRECOREATCOR15 | 337 | 343 | + | 0 | 1 | CCCGACC |
| FAM270 | RAV1AAT | 350 | 354 | + | 0 | 1 | CAACA |
| FAM002 | SORLIP1AT | 352 | 364 | + | 0 | 1 | ACAATGGCCACCG |
| FAM276 | TRANSINITDICOTS | 352 | 359 | + | 0 | 1 | ACAATGGC |
| FAM194 | PALBOXAPC | 362 | 368 | + | 0 | 1 | CCGTCCT |
| FAM324 | CGCGBOXAT | 378 | 383 | + | 0 | 1 | CCGCGC |
| FAM324 | CGCGBOXAT | 378 | 383 | − | 0 | 1 | GCGCGG |
| FAM324 | CGCGBOXAT | 380 | 385 | + | 0 | 1 | GCGCGC |
| FAM324 | CGCGBOXAT | 380 | 385 | − | 0 | 1 | GCGCGC |
| FAM194 | PALBOXAPC | 403 | 409 | + | 0 | 1 | CCGTCCT |
| FAM324 | CGCGBOXAT | 419 | 424 | + | 0 | 1 | CCGCGC |
| FAM324 | CGCGBOXAT | 419 | 424 | − | 0 | 1 | GCGCGG |
| FAM002 | SORLIP1AT | 437 | 449 | − | 0 | 1 | GGCAGCGCCACGG |
| FAM302 | SITEIIATCYTC | 470 | 480 | + | 0 | 1 | TGGGCCGTAGC |
| FAM306 | ANAERO2CONSENSUS | 484 | 489 | + | 0 | 1 | AGCAGC |
| FAM324 | CGCGBOXAT | 501 | 506 | + | 0 | 1 | GCGCGC |
| FAM324 | CGCGBOXAT | 501 | 506 | − | 0 | 1 | GCGCGC |
| FAM324 | CGCGBOXAT | 503 | 508 | + | 0 | 1 | GCGCGC |
| FAM324 | CGCGBOXAT | 503 | 508 | − | 0 | 1 | GCGCGC |
| FAM002 | SORLIP1AT | 505 | 517 | + | 0 | 1 | GCGCAGGCCACCT |
| FAM002 | BP5OSWX | 517 | 529 | + | 0 | 1 | TACAACGTGAAGC |
| FAM002 | RAV1BAT | 555 | 567 | − | 0 | 1 | GGGCACCTGCAGC |
| FAM151 | INTRONLOWER | 557 | 562 | + | 0 | 1 | TGCAGG |
| FAM013 | LTRECOREATCOR15 | 565 | 571 | + | 0 | 1 | CCCGACG |
| FAM107 | CGACGOSAMY3 | 567 | 571 | + | 0 | 1 | CGACG |
| FAM002 | ABRELATERD | 568 | 580 | + | 0 | 1 | GACGACGTGTACA |
| FAM107 | CGACGOSAMY3 | 570 | 574 | + | 0 | 1 | CGACG |
| FAM194 | PALBOXAPC | 599 | 605 | − | 0 | 1 | CCGTCCT |
| FAM324 | CGCGBOXAT | 627 | 632 | + | 0 | 1 | CCGCGC |
| FAM324 | CGCGBOXAT | 627 | 632 | − | 0 | 1 | GCGCGG |
| FAM324 | CGCGBOXAT | 629 | 634 | + | 0 | 1 | GCGCGG |
| FAM324 | CGCGBOXAT | 629 | 634 | − | 0 | 1 | CCGCGC |
| FAM069 | SURECOREATSULTR11 | 662 | 668 | − | 0 | 1 | GAGACGA |
| FAM013 | LTRECOREATCOR15 | 685 | 691 | + | 0 | 1 | TCCGACC |
| FAM107 | CGACGOSAMY3 | 702 | 706 | + | 0 | 1 | CGACG |
| FAM107 | CGACGOSAMY3 | 705 | 709 | + | 0 | 1 | CGACG |
| FAM147 | HEXAMERATH4 | 705 | 710 | − | 0 | 1 | CCGTCG |
| FAM061 | GCCCORE | 717 | 723 | + | 0 | 1 | CGCCGCC |
| FAM002 | RAV1BAT | 732 | 744 | + | 0 | 1 | GCTCACCTGCCAC |
| FAM002 | SORLIP1AT | 734 | 746 | + | 0 | 1 | TCACCTGCCACGC |
| FAM171 | MYBPZM | 745 | 751 | + | 0 | 1 | GCCTACC |
| FAM002 | TGACGTVMAMY | 754 | 766 | + | 0 | 1 | ACCTCTGACGTCG |
| FAM002 | HEXMOTIFTAH3H4 | 756 | 768 | − | 0 | 1 | GACGACGTCAGAG |
| FAM057 | ACGTCBOX | 760 | 765 | + | 0 | 1 | GACGTC |
| FAM057 | ACGTCBOX | 760 | 765 | − | 0 | 1 | GACGTC |
| FAM002 | ASF1MOTIFCAMV | 762 | 774 | − | 0 | 1 | CTCGATGACGACG |
| FAM107 | CGACGOSAMY3 | 762 | 766 | − | 0 | 1 | CGACG |
| FAM069 | SURECOREATSULTR11 | 772 | 778 | + | 0 | 1 | GAGACGC |
| FAM107 | CGACGOSAMY3 | 842 | 846 | − | 0 | 1 | CGACG |
| FAM324 | CGCGBOXAT | 864 | 869 | + | 0 | 1 | ACGCGC |
| FAM324 | CGCGBOXAT | 864 | 869 | − | 0 | 1 | GCGCGT |
| FAM324 | CGCGBOXAT | 866 | 871 | + | 0 | 1 | GCGCGT |
| FAM324 | CGCGBOXAT | 866 | 871 | − | 0 | 1 | ACGCGC |
| FAM002 | ACGTOSGLUB1 | 869 | 881 | − | 0 | 1 | CAGTACGTGTACG |
| FAM305 | ANAERO1CONSENSUS | 884 | 890 | − | 0 | 1 | AAACAAA |
| FAM107 | CGACGOSAMY3 | 914 | 918 | − | 0 | 1 | CGACG |
| FAM260 | CAREOSREP1 | 918 | 923 | − | 0 | 1 | CAACTC |
| FAM311 | EECCRCAH1 | 918 | 924 | + | 0 | 1 | GAGTTGC |
| FAM276 | TRANSINITDICOTS | 928 | 935 | − | 0 | 1 | ACGATGGC |
| FAM069 | SURECOREATSULTR11 | 932 | 938 | − | 0 | 1 | GAGACGA |
| FAM294 | CTRMCAMV35S | 935 | 943 | + | 0 | 1 | TCTCTCTCT |
| FAM294 | CTRMCAMV35S | 937 | 945 | + | 0 | 1 | TCTCTCTCT |
| FAM294 | CTRMCAMV35S | 939 | 947 | + | 0 | 1 | TCTCTCTCT |
| FAM234 | SP8BFIBSP8BIB | 955 | 961 | + | 0 | 1 | TACTATT |
| FAM324 | CGCGBOXAT | 971 | 976 | + | 0 | 1 | GCGCGC |
| FAM324 | CGCGBOXAT | 971 | 976 | − | 0 | 1 | GCGCGC |
| FAM087 | BOXIINTPATPB | 977 | 982 | − | 0 | 1 | ATAGAA |
| FAM290 | GT1GMSCAM4 | 982 | 987 | − | 0 | 1 | GAAAAA |
| FAM100 | CCAATBOX1 | 987 | 991 | + | 0 | 1 | CCAAT |
| FAM289 | LEAFYATAG | 987 | 993 | + | 0 | 1 | CCAATGT |
| FAM270 | RAV1AAT | 991 | 995 | − | 0 | 1 | CAACA |
| FAM311 | EECCRCAH1 | 995 | 1001 | + | 0 | 1 | GAGTTAC |
| FAM002 | ASF1MOTIFCAMV | 1064 | 1076 | + | 0 | 1 | TGTGGTGACGGTT |
| FAM003 | MYBPLANT | 1070 | 1080 | − | 0 | 1 | AACCAACCGTC |
| FAM171 | BOXLCOREDCPAL | 1073 | 1079 | − | 0 | 1 | ACCAACC |
| FAM002 | SORLIP1AT | 1086 | 1098 | − | 0 | 1 | GTCGCCGCCACAC |
| FAM061 | GCCCORE | 1090 | 1096 | − | 0 | 1 | CGCCGCC |
| FAM002 | ABREOSRAB21 | 1092 | 1104 | − | 0 | 1 | ACTGACGTCGCCG |
| FAM002 | HEXMOTIFTAH3H4 | 1093 | 1105 | + | 0 | 1 | GGCGACGTCAGTC |
| FAM010 | WBOXHVISO1 | 1094 | 1108 | − | 0 | 1 | CATGACTGACGTC |
| | | | | | | | GC |
| FAM002 | TGACGTVMAMY | 1095 | 1107 | − | 0 | 1 | ATGACTGACGTCG |
| FAM107 | CGACGOSAMY3 | 1095 | 1099 | + | 0 | 1 | CGACG |
| FAM057 | ACGTCBOX | 1096 | 1101 | + | 0 | 1 | GACGTC |
| FAM057 | ACGTCBOX | 1096 | 1101 | − | 0 | 1 | GACGTC |
| FAM304 | OSE2ROOTNODULE | 1113 | 1117 | − | 0 | 1 | CTCTT |
| FAM026 | RYREPEATGMGY2 | 1122 | 1132 | − | 0 | 1 | CCCATGCATTC |
| FAM024 | CANBNNAPA | 1132 | 1140 | − | 0 | 1 | CCAACACCC |
| FAM270 | RAV1AAT | 1135 | 1139 | − | 0 | 1 | CAACA |
| FAM324 | CGCGBOXAT | 1177 | 1182 | + | 0 | 1 | GCGCGC |
| FAM324 | CGCGBOXAT | 1177 | 1182 | − | 0 | 1 | GCGCGC |
| FAM147 | HEXAMERATH4 | 1186 | 1191 | + | 0 | 1 | CCGTCG |
| FAM002 | ASF1MOTIFCAMV | 1187 | 1199 | − | 0 | 1 | AGCCATGACGACG |
| FAM107 | CGACGOSAMY3 | 1187 | 1191 | − | 0 | 1 | CGACG |
| FAM107 | CGACGOSAMY3 | 1200 | 1204 | + | 0 | 1 | CGACG |
| FAM324 | CGCGBOXAT | 1226 | 1231 | + | 0 | 1 | CCGCGC |
| FAM324 | CGCGBOXAT | 1226 | 1231 | − | 0 | 1 | GCGCGG |
| FAM324 | CGCGBOXAT | 1228 | 1233 | + | 0 | 1 | GCGCGC |
| FAM324 | CGCGBOXAT | 1228 | 1233 | − | 0 | 1 | GCGCGC |
| FAM015 | ACGTABOX | 1253 | 1258 | + | 0 | 1 | TACGTA |
| FAM015 | ACGTABOX | 1253 | 1258 | − | 0 | 1 | TACGTA |
| FAM002 | SORLIP1AT | 1294 | 1306 | − | 0 | 1 | CTCTTCGCCACCC |
| FAM002 | SORLIP1AT | 1301 | 1313 | + | 0 | 1 | GAAGAGGCCACGG |
| FAM304 | OSE2ROOTNODULE | 1302 | 1306 | − | 0 | 1 | CTCTT |
| FAM302 | SORLIP2AT | 1307 | 1317 | − | 0 | 1 | CGGGCCGTGGC |
| FAM013 | LTRECOREATCOR15 | 1314 | 1320 | + | 0 | 1 | CCCGACC |
| FAM002 | SORLIP1AT | 1339 | 1351 | + | 0 | 1 | CATCTCGCCACCA |
| FAM089 | BS1EGCCR | 1359 | 1364 | − | 0 | 1 | AGCGGG |
| FAM002 | SORLIP1AT | 1366 | 1378 | + | 0 | 1 | GCCGCTGCCACCG |
| FAM270 | RAV1AAT | 1381 | 1385 | − | 0 | 1 | CAACA |
| FAM228 | SEF3MOTIFGM | 1384 | 1389 | − | 0 | 1 | AACCCA |
| FAM171 | MYBPZM | 1386 | 1392 | − | 0 | 1 | CCCAACC |
| FAM302 | SITEIIATCYTC | 1389 | 1399 | + | 0 | 1 | TGGGCTGAAGC |
| FAM010 | QELEMENTZMZM13 | 1403 | 1417 | − | 0 | 1 | AAAGGTCACGGGC |
| | | | | | | | TT |
| FAM205 | PYRIMIDINEBOXOSRAM | 1413 | 1418 | + | 0 | 1 | CCTTTT |
| FAM290 | GT1GMSCAM4 | 1415 | 1420 | − | 0 | 1 | GAAAAA |
| FAM290 | GT1GMSCAM4 | 1421 | 1426 | − | 0 | 1 | GAAAAA |
| FAM267 | TAAAGSTKST1 | 1426 | 1432 | − | 0 | 1 | AATAAAG |
| FAM027 | -10PEHVPSBD | 1429 | 1434 | + | 0 | 1 | TATTCT |
| FAM205 | PYRIMIDINEBOXOSRAM | 1437 | 1442 | + | 0 | 1 | CCTTTT |
| FAM270 | RAV1AAT | 1466 | 1470 | − | 0 | 1 | CAACA |
| FAM202 | -300ELEMENT | 1469 | 1477 | + | 0 | 1 | TGCAAAATC |
| FAM267 | TAAAGSTKST1 | 1499 | 1505 | + | 0 | 1 | TATAAAG |
| FAM267 | NTBBF1ARROLB | 1500 | 1506 | − | 0 | 1 | ACTTTAT |
| FAM270 | RAV1AAT | 1514 | 1518 | − | 0 | 1 | CAACA |
| FAM267 | TAAAGSTKST1 | 1542 | 1548 | − | 0 | 1 | ATTAAAG |
| FAM221 | S1FBOXSORPS1L21 | 1551 | 1556 | + | 0 | 1 | ATGGTA |
| FAM243 | TATABOX4 | 1555 | 1561 | + | 0 | 1 | TATATAA |
| FAM172 | MYCATERD | 1564 | 1570 | − | 0 | 1 | CATGTGA |
| FAM172 | MYCATRD2 | 1565 | 1571 | + | 0 | 1 | CACATGT |
| FAM241 | TATABOX2 | 1577 | 1583 | + | 0 | 1 | TATAAAT |
| FAM087 | BOXIINTPATPB | 1596 | 1601 | − | 0 | 1 | ATAGAA |
| FAM304 | OSE2ROOTNODULE | 1611 | 1615 | + | 0 | 1 | CTCTT |
| FAM099 | CCA1ATLHCB1 | 1620 | 1627 | − | 0 | 1 | AACAATCT |
| FAM241 | TATABOX2 | 1628 | 1634 | + | 0 | 1 | TATAAAT |
| FAM010 | WBOXHVISO1 | 1647 | 1661 | − | 0 | 1 | TATGACTTTTAAGAT |
| FAM087 | BOXIINTPATPB | 1667 | 1672 | + | 0 | 1 | ATAGAA |
| FAM290 | GT1GMSCAM4 | 1670 | 1675 | + | 0 | 1 | GAAAAA |
| FAM305 | ANAERO1CONSENSUS | 1698 | 1704 | + | 0 | 1 | AAACAAA |
| FAM202 | -300ELEMENT | 1711 | 1719 | − | 0 | 1 | TGAAAAGTT |
| FAM267 | TAAAGSTKST1 | 1740 | 1746 | − | 0 | 1 | CATAAAG |
| FAM026 | RYREPEATGMGY2 | 1742 | 1752 | − | 0 | 1 | TGCATGCATAA |
| FAM026 | RYREPEATVFLEB4 | 1745 | 1755 | + | 0 | 1 | TGCATGCATGC |
| FAM026 | RYREPEATVFLEB4 | 1746 | 1756 | − | 0 | 1 | TGCATGCATGC |
| FAM026 | RYREPEATBNNAPA | 1749 | 1759 | + | 0 | 1 | TGCATGCAACT |
| FAM325 | MYBCOREATCYCB1 | 1772 | 1776 | + | 0 | 1 | AACGG |
| FAM002 | SORLIP1AT | 1802 | 1814 | + | 0 | 1 | TGGGCGGCCACGT |
| FAM061 | GCCCORE | 1804 | 1810 | − | 0 | 1 | GGCCGCC |
| FAM002 | ABREOSRAB21 | 1806 | 1818 | − | 0 | 1 | GGCGACGTGGCCG |
| FAM002 | ABREOSRAB21 | 1807 | 1819 | + | 0 | 1 | GGCCACGTCGCCG |
| FAM107 | CGACGOSAMY3 | 1812 | 1816 | − | 0 | 1 | CGACG |
| FAM061 | GCCCORE | 1815 | 1821 | + | 0 | 1 | CGCCGCC |
| FAM002 | TGACGTVMAMY | 1827 | 1839 | + | 0 | 1 | GGAACTGACGTGT |
| FAM002 | HEXMOTIFTAH3H4 | 1829 | 1841 | − | 0 | 1 | GGACACGTCAGTT |
| FAM002 | GADOWNAT | 1830 | 1842 | + | 0 | 1 | ACTGACGTGTCCC |
| FAM302 | SORLIP2AT | 1838 | 1848 | − | 0 | 1 | CGGGCCGGGAC |
| FAM013 | LTRECOREATCOR15 | 1845 | 1851 | + | 0 | 1 | CCCGACG |
| FAM107 | CGACGOSAMY3 | 1847 | 1851 | + | 0 | 1 | CGACG |
| FAM107 | CGACGOSAMY3 | 1850 | 1854 | + | 0 | 1 | CGACG |
| FAM107 | CGACGOSAMY3 | 1853 | 1857 | + | 0 | 1 | CGACG |
| FAM107 | CGACGOSAMY3 | 1856 | 1860 | + | 0 | 1 | CGACG |
| FAM324 | CGCGBOXAT | 1871 | 1876 | + | 0 | 1 | CCGCGC |
| FAM324 | CGCGBOXAT | 1871 | 1876 | − | 0 | 1 | GCGCGG |
| FAM324 | CGCGBOXAT | 1876 | 1881 | + | 0 | 1 | CCGCGC |
| FAM324 | CGCGBOXAT | 1876 | 1881 | − | 0 | 1 | GCGCGG |
| FAM304 | OSE2ROOTNODULE | 1889 | 1893 | + | 0 | 1 | CTCTT |
| FAM151 | INTRONLOWER | 1910 | 1915 | + | 0 | 1 | TGCAGG |

7) p-MAEM1

PLACE analysis results of p-MAEM1 are listed in Table 55. No TATA box motifs are found in this promoter. One CAAT box motif is located at nucleotide position 655-659 of the forward strand.

TABLE 55
PLACE analysis results of the 922 bp promoter p-MAEM1

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM027 | -10PEHVPSBD | 8 | 13 | − | 0 | 1 | TATTCT |
| FAM304 | OSE2ROOTNODULE | 52 | 56 | − | 0 | 1 | CTCTT |
| FAM310 | CPBCSPOR | 105 | 110 | + | 0 | 1 | TATTAG |
| FAM087 | BOXIINTPATPB | 160 | 165 | + | 0 | 1 | ATAGAA |
| FAM271 | SEBFCONSSTPR10A | 168 | 174 | + | 0 | 1 | TTGTCAC |
| FAM322 | BIHD1OS | 169 | 173 | + | 0 | 1 | TGTCA |
| FAM012 | IBOXCORE | 208 | 214 | + | 0 | 1 | GATAAAT |
| FAM273 | TATCCAOSAMY | 216 | 222 | − | 0 | 1 | TATCCAA |
| FAM014 | MYBST1 | 217 | 223 | + | 0 | 1 | TGGATAC |
| FAM292 | PREATPRODH | 236 | 241 | + | 0 | 1 | ACTCAT |
| FAM027 | -10PEHVPSBD | 244 | 249 | − | 0 | 1 | TATTCT |
| FAM107 | CGACGOSAMY3 | 253 | 257 | + | 0 | 1 | CGACG |
| FAM267 | TAAAGSTKST1 | 298 | 304 | + | 0 | 1 | ACTAAAG |
| FAM263 | DPBFCOREDCDC3 | 313 | 319 | + | 0 | 1 | ACACACG |
| FAM014 | MYBST1 | 362 | 368 | − | 0 | 1 | TGGATAT |
| FAM025 | AMYBOX2 | 363 | 369 | + | 0 | 1 | TATCCAT |
| FAM273 | TATCCAOSAMY | 363 | 369 | + | 0 | 1 | TATCCAT |
| FAM015 | ACGTABOX | 393 | 398 | + | 0 | 1 | TACGTA |
| FAM015 | ACGTABOX | 393 | 398 | − | 0 | 1 | TACGTA |
| FAM202 | -300ELEMENT | 422 | 430 | + | 0 | 1 | TGAAAAATT |
| FAM290 | GT1GMSCAM4 | 423 | 428 | + | 0 | 1 | GAAAAA |
| FAM305 | ANAERO1CONSENSUS | 433 | 439 | + | 0 | 1 | AAACAAA |
| FAM039 | AACACOREOSGLUB1 | 434 | 440 | + | 0 | 1 | AACAAAC |
| FAM026 | RYREPEATGMGY2 | 493 | 503 | − | 0 | 1 | CCCATGCATCG |
| FAM002 | T/GBOXATPIN2 | 505 | 517 | + | 0 | 1 | GGAAACGTGGACA |
| FAM002 | SITEIOSPCNA | 540 | 552 | − | 0 | 1 | GACCAGGTGGGTT |
| FAM228 | SEF3MOTIFGM | 540 | 545 | + | 0 | 1 | AACCCA |
| FAM002 | RAV1BAT | 541 | 553 | + | 0 | 1 | ACCCACCTGGTCC |
| FAM002 | CACGTGMOTIF | 572 | 584 | − | 0 | 1 | TGCCACGTGTATC |
| FAM002 | ABREATRD2 | 573 | 585 | + | 0 | 1 | ATACACGTGGCAC |
| FAM263 | DPBFCOREDCDC3 | 575 | 581 | + | 0 | 1 | ACACGTG |
| FAM002 | SORLIP1AT | 577 | 589 | − | 0 | 1 | CAGCGTGCCACGT |
| FAM010 | WBOXATNPR1 | 613 | 627 | − | 0 | 1 | CTTGACACGTTAGCT |
| FAM002 | GADOWNAT | 614 | 626 | + | 0 | 1 | GCTAACGTGTCAA |
| FAM322 | BIHD1OS | 621 | 625 | + | 0 | 1 | TGTCA |
| FAM263 | DPBFCOREDCDC3 | 624 | 630 | − | 0 | 1 | ACACTTG |
| FAM002 | SORLIP1AT | 627 | 639 | − | 0 | 1 | GGGCCGGCCACAC |
| FAM302 | SORLIP2AT | 630 | 640 | − | 0 | 1 | GGGGCCGGCCA |
| FAM151 | INTRONLOWER | 639 | 644 | − | 0 | 1 | TGCAGG |
| FAM107 | CGACGOSAMY3 | 648 | 652 | − | 0 | 1 | CGACG |
| FAM100 | CCAATBOX1 | 655 | 659 | + | 0 | 1 | CCAAT |
| FAM228 | SEF3MOTIFGM | 680 | 685 | + | 0 | 1 | AACCCA |
| FAM061 | GCCCORE | 694 | 700 | + | 0 | 1 | TGCCGCC |
| FAM061 | GCCCORE | 697 | 703 | + | 0 | 1 | CGCCGCC |
| FAM194 | PALBOXAPC | 702 | 708 | + | 0 | 1 | CCGTCCG |
| FAM302 | SORLIP2AT | 706 | 716 | − | 0 | 1 | GGGGCCGGCGG |
| FAM002 | ACGTOSGLUB1 | 724 | 736 | + | 0 | 1 | TTGTACGTGCACC |
| FAM002 | ASF1MOTIFCAMV | 743 | 755 | − | 0 | 1 | ATCGATGACGATG |
| FAM307 | ANAERO3CONSENSUS | 755 | 761 | + | 0 | 1 | TCATCAC |
| FAM094 | CACGCAATGMGH3 | 761 | 768 | + | 0 | 1 | CACGCAAT |
| FAM263 | DPBFCOREDCDC3 | 771 | 777 | + | 0 | 1 | ACACAAG |
| FAM302 | SITEIIATCYTC | 785 | 795 | − | 0 | 1 | TGGGCTGTTTA |
| FAM002 | ASF1MOTIFCAMV | 846 | 858 | − | 0 | 1 | GCATGTGACGACA |
| FAM172 | MYCATERD | 851 | 857 | − | 0 | 1 | CATGTGA |
| FAM026 | RYREPEATLEGUMINBOX | 852 | 862 | + | 0 | 1 | CACATGCACAT |
| FAM172 | MYCATRD2 | 852 | 858 | + | 0 | 1 | CACATGC |
| FAM267 | TAAAGSTKST1 | 870 | 876 | + | 0 | 1 | CATAAAG |
| FAM304 | OSE2ROOTNODULE | 874 | 878 | − | 0 | 1 | CTCTT |

8) p-MAEM20

PLACE analysis results of p-MAEM20 are listed in Table 56. No TATA box motifs are found in this promoter. One CAAT box motif is located at nucleotide position 668-672 of the reverse strand.

TABLE 56
PLACE analysis results of the 698 bp promoter p-MAEM20

| | | | | | | | |
|---|---|---|---|---|---|---|---|
| IUPAC | | Start | End | | | | |
| Family | IUPAC | pos. | pos. | Strand | Mismatches | Score | Sequence |

| | | | | | | | |
|---|---|---|---|---|---|---|---|
| FAM262 | CIACADIANLELHC | 3 | 12 | − | 0 | 1 | CAACTTAATC |
| FAM010 | WBOXATNPR1 | 9 | 23 | + | 0 | 1 | GTTGACTGGCAAATT |
| FAM012 | IBOXCORE | 32 | 38 | + | 0 | 1 | GATAATA |
| FAM012 | IBOXCORE | 55 | 61 | + | 0 | 1 | GATAACC |
| FAM266 | MYB1AT | 57 | 62 | + | 0 | 1 | TAACCA |
| FAM003 | MYBPLANT | 68 | 78 | + | 0 | 1 | CACCAACCGAC |
| FAM171 | BOXLCOREDCPAL | 69 | 75 | + | 0 | 1 | ACCAACC |
| FAM013 | DRE2COREZMRAB17 | 73 | 79 | + | 0 | 1 | ACCGACT |
| FAM270 | RAV1AAT | 80 | 84 | − | 0 | 1 | CAACA |
| FAM303 | OSE1ROOTNODULE | 95 | 101 | − | 0 | 1 | AAAGATC |
| FAM021 | GT1CORE | 96 | 106 | − | 0 | 1 | TGGTTAAAGAT |
| FAM267 | TAAAGSTKST1 | 98 | 104 | − | 0 | 1 | GTTAAAG |
| FAM266 | MYB1AT | 101 | 106 | + | 0 | 1 | TAACCA |
| FAM008 | MYB2AT | 116 | 126 | + | 0 | 1 | TAACTAACTGT |
| FAM281 | MYB1LEPR | 117 | 123 | − | 0 | 1 | GTTAGTT |
| FAM270 | RAV1AAT | 124 | 128 | − | 0 | 1 | CAACA |
| FAM270 | RAV1AAT | 127 | 131 | − | 0 | 1 | CAACA |
| FAM069 | SURECOREATSULTR11 | 154 | 160 | + | 0 | 1 | GAGACTT |
| FAM245 | TBOXATGAPB | 157 | 162 | + | 0 | 1 | ACTTTG |
| FAM234 | SP8BFIBSP8BIB | 186 | 192 | + | 0 | 1 | TACTATT |
| FAM015 | ACGTABOX | 205 | 210 | + | 0 | 1 | TACGTA |
| FAM015 | ACGTABOX | 205 | 210 | − | 0 | 1 | TACGTA |
| FAM116 | DRE1COREZMRAB17 | 217 | 223 | − | 0 | 1 | ACCGAGA |
| FAM010 | WBOXHVISO1 | 253 | 267 | + | 0 | 1 | GGTGACTGACAGACT |
| FAM322 | BIHD1OS | 259 | 263 | − | 0 | 1 | TGTCA |
| FAM010 | WBBOXPCWRKY1 | 290 | 304 | − | 0 | 1 | TTTGACTAGAACAAG |
| FAM324 | CGCGBOXAT | 336 | 341 | + | 0 | 1 | GCGCGC |
| FAM324 | CGCGBOXAT | 336 | 341 | − | 0 | 1 | GCGCGC |
| FAM324 | CGCGBOXAT | 338 | 343 | + | 0 | 1 | GCGCGC |
| FAM324 | CGCGBOXAT | 338 | 343 | − | 0 | 1 | GCGCGC |
| FAM304 | OSE2ROOTNODULE | 348 | 352 | + | 0 | 1 | CTCTT |
| FAM012 | IBOXCORE | 351 | 357 | − | 0 | 1 | GATAAAA |
| FAM014 | SREATMSD | 352 | 358 | + | 0 | 1 | TTTATCC |
| FAM014 | MYBST1 | 353 | 359 | − | 0 | 1 | AGGATAA |
| FAM263 | DPBFCOREDCDC3 | 357 | 363 | − | 0 | 1 | ACACAGG |
| FAM278 | UPRMOTIFIIAT | 357 | 375 | + | 0 | 1 | CCTGTGTGTCTCCTCC |
| | | | | | | | ACG |
| FAM069 | ARFAT | 362 | 368 | + | 0 | 1 | GTGTCTC |
| FAM069 | SURECOREATSULTR11 | 362 | 368 | − | 0 | 1 | GAGACAC |
| FAM002 | ASF1MOTIFCAMV | 381 | 393 | − | 0 | 1 | TCTCATGACGCCT |
| FAM107 | CGACGOSAMY3 | 402 | 406 | + | 0 | 1 | CGACG |
| FAM026 | RYREPEATBNNAPA | 407 | 417 | + | 0 | 1 | ACCATGCAGTG |
| FAM324 | CGCGBOXAT | 426 | 431 | + | 0 | 1 | CCGCGC |
| FAM324 | CGCGBOXAT | 426 | 431 | − | 0 | 1 | GCGCGG |
| FAM324 | CGCGBOXAT | 428 | 433 | + | 0 | 1 | GCGCGC |
| FAM324 | CGCGBOXAT | 428 | 433 | − | 0 | 1 | GCGCGC |
| FAM324 | CGCGBOXAT | 430 | 435 | + | 0 | 1 | GCGCGT |
| FAM324 | CGCGBOXAT | 430 | 435 | − | 0 | 1 | ACGCGC |
| FAM061 | GCCCORE | 457 | 463 | − | 0 | 1 | GGCCGCC |
| FAM002 | ABRELATERD | 475 | 487 | − | 0 | 1 | GGCGACGTGGTAA |
| FAM002 | ABREOSRAB21 | 476 | 488 | + | 0 | 1 | TACCACGTCGCCC |
| FAM107 | CGACGOSAMY3 | 481 | 485 | − | 0 | 1 | CGACG |
| FAM002 | ABRE3HVA1 | 507 | 519 | + | 0 | 1 | AGCAACGTGTCGA |
| FAM261 | CDTDREHVCBF2 | 515 | 520 | + | 0 | 1 | GTCGAC |
| FAM261 | CDTDREHVCBF2 | 515 | 520 | − | 0 | 1 | GTCGAC |
| FAM002 | TGACGTVMAMY | 527 | 539 | + | 0 | 1 | GCCTCTGACGTGT |
| FAM002 | HEXMOTIFTAH3H4 | 529 | 541 | − | 0 | 1 | GGACACGTCAGAG |
| FAM002 | GADOWNAT | 530 | 542 | + | 0 | 1 | TCTGACGTGTCCC |
| FAM194 | PALBOXAPC | 545 | 551 | + | 0 | 1 | CCGTCCT |
| FAM205 | PYRIMIDINEBOXOSRAM | 558 | 563 | − | 0 | 1 | CCTTTT |
| FAM107 | CGACGOSAMY3 | 595 | 599 | − | 0 | 1 | CGACG |
| FAM304 | OSE2ROOTNODULE | 660 | 664 | − | 0 | 1 | CTCTT |
| FAM100 | CCAATBOX1 | 668 | 672 | − | 0 | 1 | CCAAT |

### Example 11

### Binary Vector Construction for Maize Transformation to Evaluate the Function of the Promoters

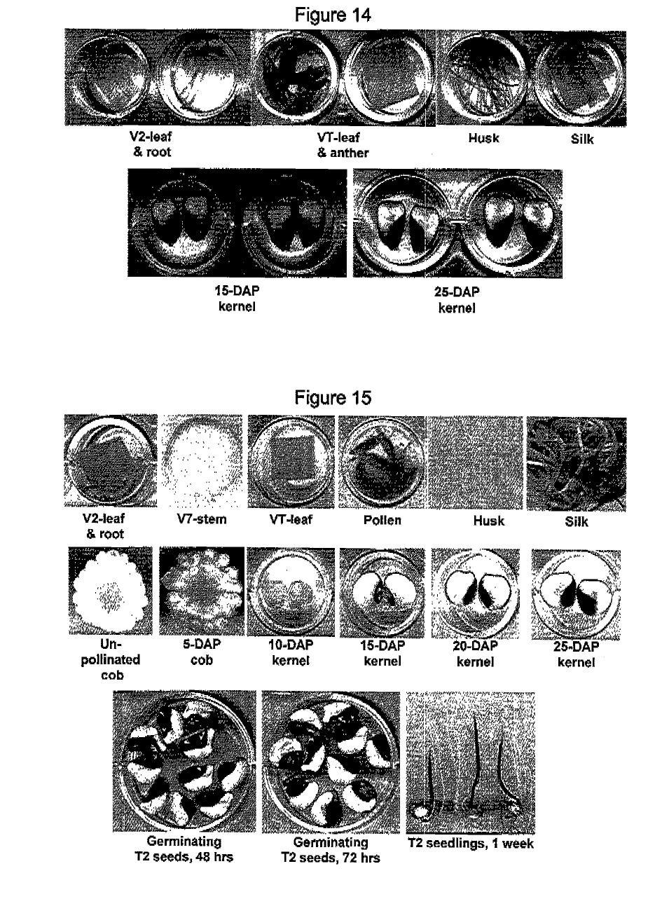

To facilitate subcloning, the promoter fragments of MAWS23, 27, 30, 57, 60, 63, MAEM1 and MAEM20 were modified by the addition of a SwaI restriction enzyme site at its 5′ end and a Bs/WI site at its 3′ end. The SwaI-p-MA promoter-BsiWI fragment was digested and ligated into a SwaI and BsiWI digested BPS basic binary vector RCB1006 that comprises a plant selectable marker expression cassette (p-Ubi::AHAS::t-XI12), as well as a promoter evaluation cassette that consists of a multiple cloning site (MCS) for insertion of promoter and the rice MET1-1 intron to supply intron-mediated enhancement in monocot cells, GUS reporter gene, and NOS terminator. Diagram of RCB1006 is shown in FIG. 13.

Table 57 lists the resulting binary vectors of the MA promoters, Sequences of the promoter cassettes in the binary vectors are shown in SEQ ID NOs: 55, 56, 59-61, 69-71.

TABLE 57
Binary vectors of the MA promoters for corn transformation

| Promoter | Vector | | SEQ |
|---|---|---|---|
| ID | ID | Description | ID NO |
| p-MAWS23 | RTP1060 | p_MAWS23::iMET1::GUS::t-NOS | 69 |
| p-MAWS27 | RTP1059 | p_MAWS27::iMET1::GUS::t-NOS | 60 |
| p-MAWS30 | RTP1053 | p_MAWS30::iMET1::GUS::t-NOS | 70 |
| p-MAWS57 | RTP1049 | p_MAWS57::iMET1::GUS::t-NOS | 71 |
| p-MAWS60 | RTP1056 | p_MAWS60::iMET1::GUS::t-NOS | 55 |
| p-MAWS63 | RTP1048 | p_MAWS63::iMET1::GUS::t-NOS | 61 |
| p-MAEM1 | RTP1061 | p_MAEM1::iMET1::GUS::t-NOS | 56 |
| p-MAEM20 | RTP1064 | p_MAEM20::iMET1::GUS::t-NOS | 59 |

### Example 12

### Promoter Evaluation in Transgenic Maize with the MA Promoters

Expression patterns and levels driven by the MA promoters were measured using GUS histochemical analysis following the protocol in the art (Jefferson 1987). Maize transformation was conducted using an Agrobacterium-mediated transformation system. Ten and five single copy events for T0 and T1 plants were chosen for the promoter analysis. GUS expression was measured at various developmental stages:

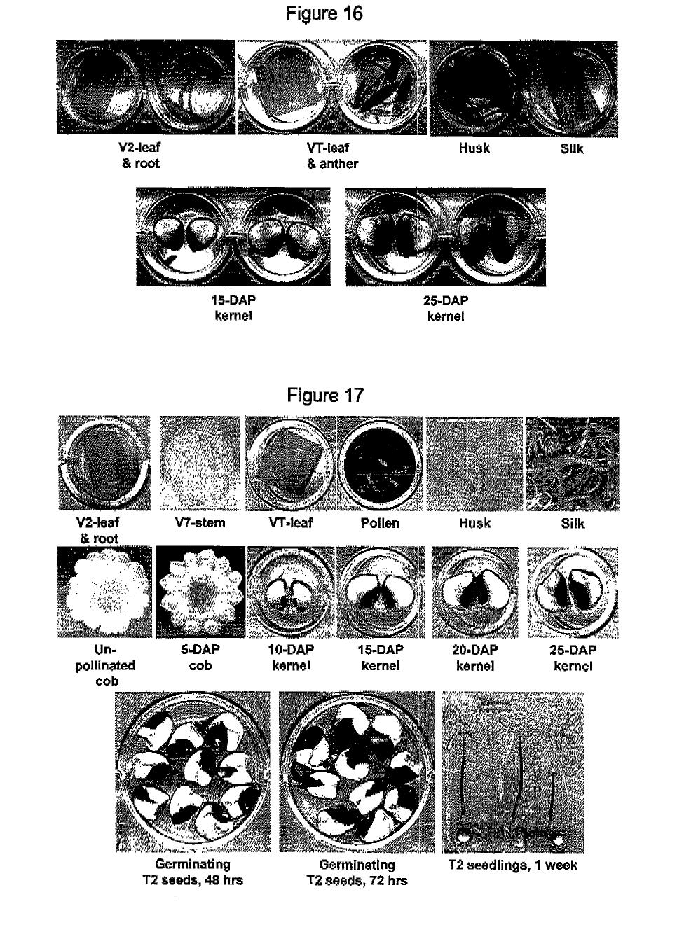

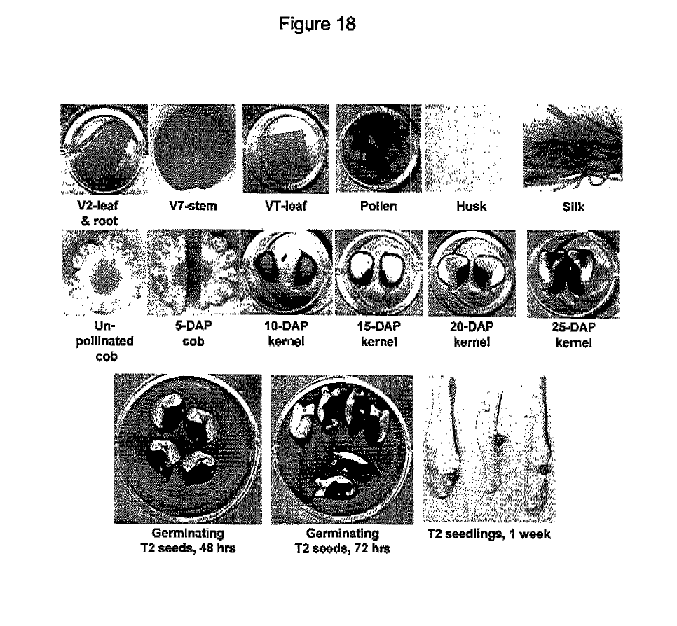

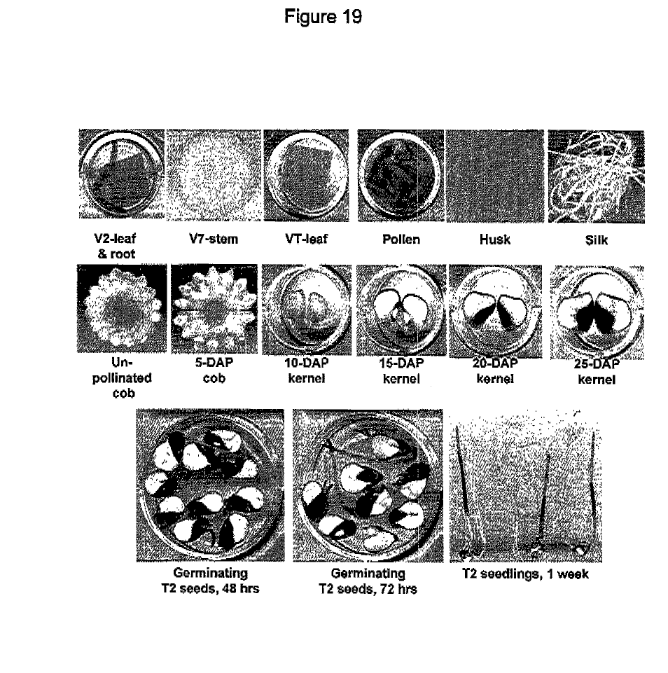

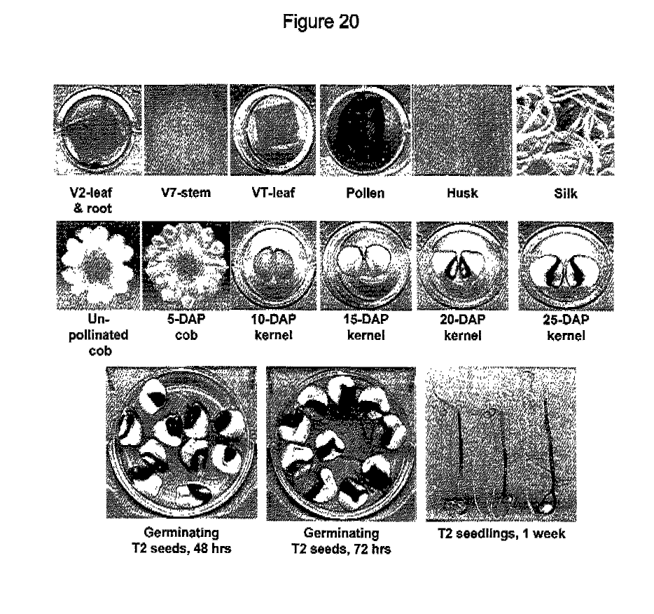

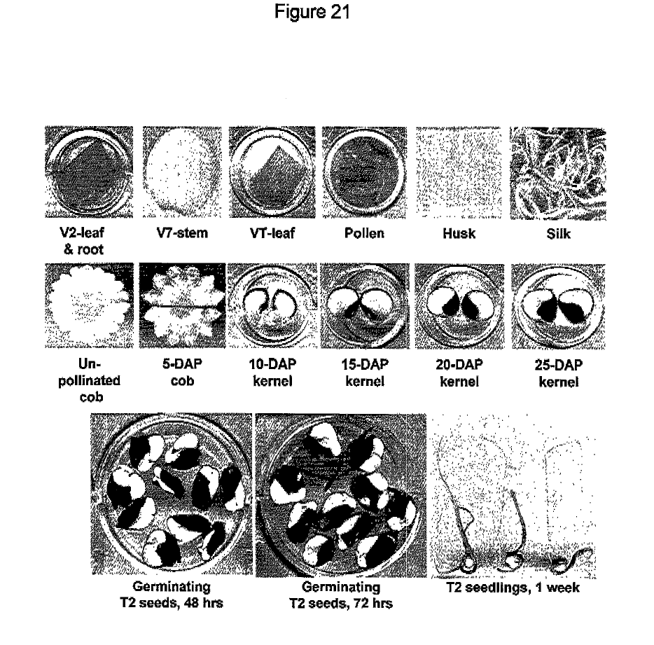

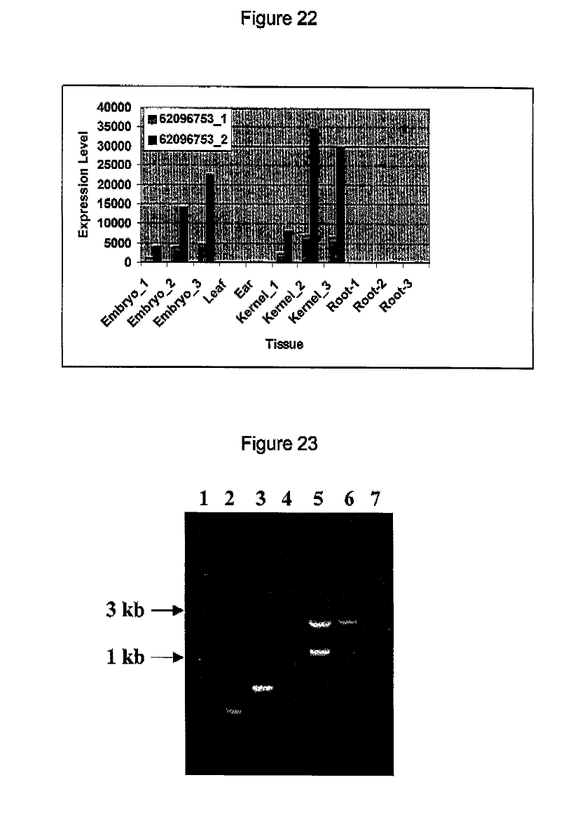

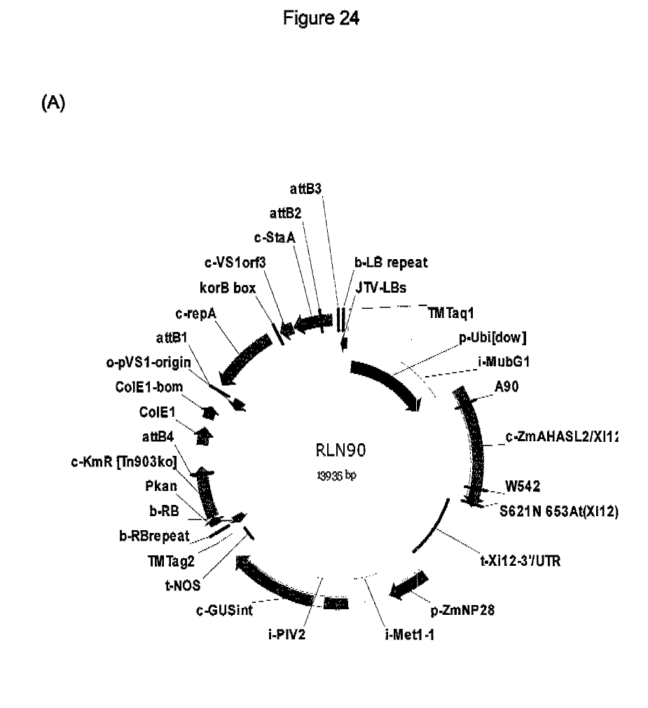

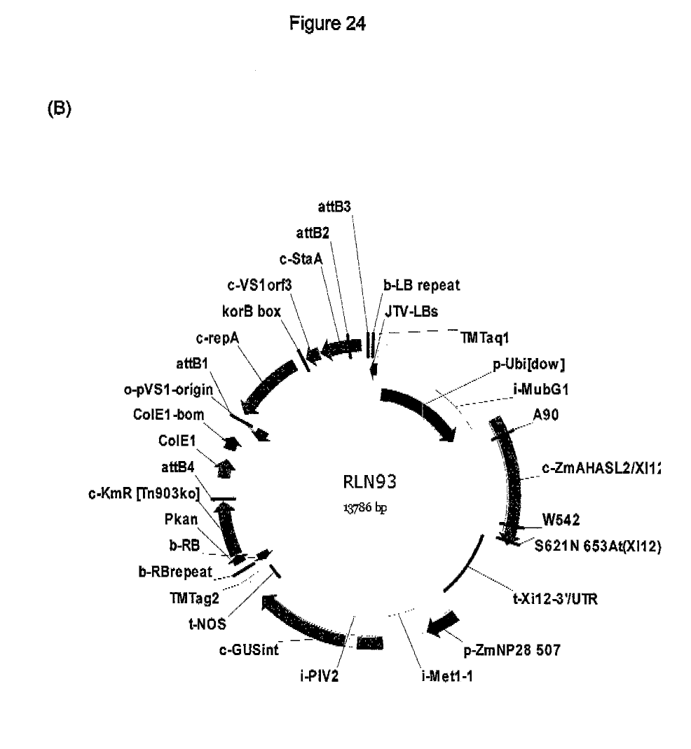

1) Roots and leaves at 5-leaf stage
2) Stem at V-7 stage
2) Leaves, husk and silk at flowering stage (first emergence of silk)
3) Spikelets/Tassel (at pollination)
5) Ear or Kernels at 5, 10, 15, 20, and 25 days after pollination (DAP)
The results indicated that all these 9 promoters expressed specifically in pollen and in embryo (FIGS. 14 to 21).

### Example 13

### Identification of Maize Promoter pZmNP28

Based on an Affymetrix GeneChip® Wheat Genome Arrays experiment carried out using methods well-known to the persons skilled in the art, a transcript, Ta.4874.1.S1_at was selected as drought inducible expression. In brief, Affymetrix GeneChip® Wheat Genome Arrays were interrogated with probes derived from different RNA samples (stems, leaves, roots, drought-stressed roots and drought-stressed leaves) and candidate genes exhibiting drought inducible expression profile were identified. Stems, leaves and roots at normal growth condition and drought-stressed conditions were harvested, RNA was extracted and further purified, and the quality and yield of RNA was confirmed by techniques known in the art. The RNA was labeled and hybridized to GeneChip® Wheat Genome Arrays and the data analyzed to derive lists of genes in rank order. Microarray expression was analyzed using AVADIS™ software (Strand Genomics Pvt. Ltd. Bangalore). The raw data for all microarray analysis were imported into AVADIS and the RMA algorithm (Irazarry et al., Biostatistics 4(2): 249-264, 2003) was applied for background correction, normalization and probe aggregation. Absolute calls and p-values were generated for each gene and all probe sets that did not hybridize to nucleic acid in a sample, i.e., were absent (absolute call), across all arrays were removed from the analysis. For determination of transcripts preferentially or selectively expressed in drought-stressed roots and leaves, differential expression analyses were conducted where normal grown stem, leaves and roots were compared to drought-stressed roots and leaves. Ta.4874.1.S1_at showing 10-fold greater expression in drought-stressed leaves than in other tissues was selected as a drought inducible transcript.

The sequence of Ta.14617.1.S1_at was aligned to the sequences of the Affymetrix maize chip. The maize Zm.8705.1.S1_at was identified as an ortholog based on nucleotide sequence identity at 76% to part of the Ta.4874.1.S1_at. The sequence of Zm.8705.1.S1_at is shown in SEQ ID NO: 108.

### Example 14

### The Expression Patterns of Zm.8705.1.S1_at in Maize

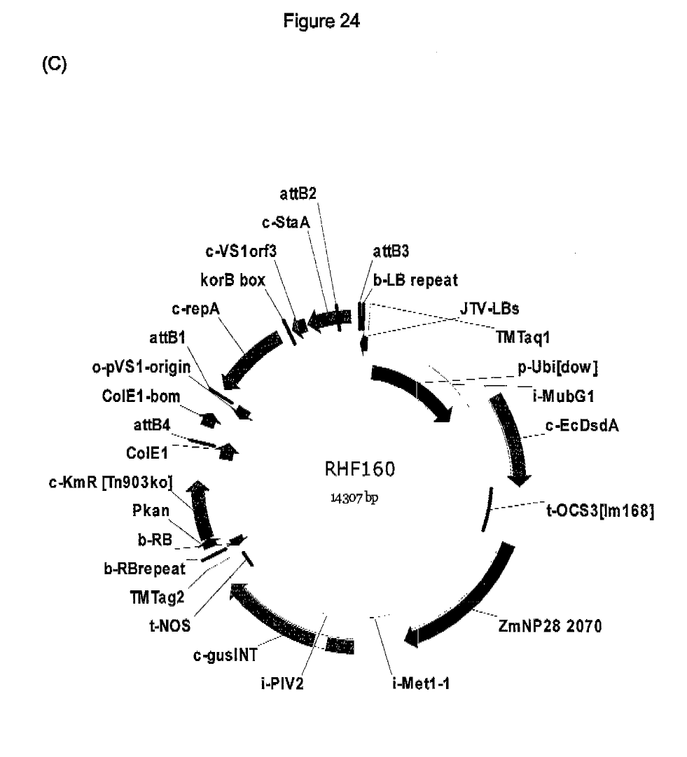

Analysis of 36 Affymetrix maize chips including immature embryo (6), leaf (8), ear (11), and kernel (11) indicated that Zm.8705.1.S1_at expressed specifically in immature embryo and in kernel (FIG. 22).

### Example 15

### Validation of the Expression Pattern of Zm.8705.1.S1_at the mRNA Levels

Quantitative RT-PCR (qRT-PCR) was performed to validate expression of Zm.8705.1.S1_at gene in various types of tissues. To find mRNA sequence with better quality for designing qRT-PCR primers, the sequence of Zm.8705.1.S1_at was blasted against the BPS in-house Hyseq database. One Hyseq maize EST ZM06MC34918_{—}62096753 (846 bp) was identified to be the same gene as Zm.8705.1.S1_at. The sequence of ZM06MC34918_{—}62096753 is shown in SEQ ID NO: 108.

Primers for qRT-PCR were designed using the Vector NTI software package (Invitrogen, Carlsbad, Calif., USA). Two sets of primers were used for PCR amplification. The sequences of primers are in Table 58.

TABLE 58
Primer sequences for RT-QPCR

| Primer | Sequence |
|---|---|
| ZM06MC34918_62096753_Forward_1 | CTCAAGGACGAGCTGACGA |
| | GCAT |
| ZM06MC34918_62096753_Reverse_1 | TAGCCCGGACGAGTCTCCT |
| | GAA |
| ZM06MC34918_62096753_Forward_2 | CAAGGACGAGCTGACGAGC |
| | AT |
| ZM06MC34918_62096753_Reverse_2 | CCCGGACGAGTCTCCTGAA |
| | A |
| GAPDH_Forward | GTAAAGTTCTTCCTGATCT |
| | GAAT |
| GAPDH_Reverse | TCGGAAGCAGCCTTAATA |

qRT-PCR was performed using SuperScript III Reverse Transcriptase (Invitrogen, Carlsbad, Calif., USA) and SYBR Green QPCR Master Mix (Eurogentec, San Diego, Calif., USA) in an ABI Prism 7000 sequence detection system. cDNA was synthesized using 2-3 μg of total RNA and 1 μL reverse transcriptase in a 20 μL volume. The cDNA was diluted to a range of concentrations (15-20 ng/μL). Thirty to forty ng of cDNA was used for QPCR in a 30 μL volume with SYBR Green QPCR Master Mix following the manufacturer's instruction. The thermocycling conditions were as follows: hold at 50° C. for 2 minutes and at 95° C. for 10 minutes, 40 cycles of 95° C. for 15 seconds and 60° C. for 1 minute for amplification. After the final cycle of the amplification, the dissociation curve analysis was carried out to verify that the amplification occurred specifically and no primer dimer was produced during the amplification process. The housekeeping gene glyceraldehyde-3-phosphate-dehydrogenase (GAPDH, Table 58 for primer sequences) was used as an endogenous reference gene to normalize the calculation using Comparative Ct (Cycle of threshold) value method. The ΔC_{T} value was obtained by subtracting the GAPDH Ct value from the candidate gene (ZM06MC34918_{—}62096753) Ct value of the same samples. The relative mRNA expression level of the gene candidate was given by 2^{-ΔCT}. The qRT-PCR results are summarized in FIG. 22. Both primer sets gave embryo-specifical expression patterns that are validated to the expression patterns obtained from the maize Affymetrix chip analysis.

### Example 16

### Annotation of the Zm.8705.1.S1_at

The sequence of the maize EST ZM06MC34918_{—}62096753 was searched via BlastX. The EST did not hit to any known maize gene. The 20 homologues of EST ZM06MC34918_{—}62096753 with the highest score are listed in Table 59.

TABLE 59
Zm.8705.1.S1_at gene annotation

| Accession | Description | Species | Score | E-value |
|---|---|---|---|---|
| AAL23749.1 | stress-inducible membrane | Bromus inermis | 259 | 2.00E−67 |
| | pore protein | | | |
| NP_001042833.1 | Os01g0303300 | Oryza sativa | 265 | 7.00E−65 |
| ABE83193.1 | Mitochondrial import inner | Medicago truncatula | 200 | 9.00E−50 |
| | membrane translocase, | | | |
| | subunit Tim17/22 | | | |
| AAV84280.1 | dehydration up-regulated putative | | 199 | 1.00E−49 |
| | membrane pore protein | | | |
| NP_001049884.1 | Os03g0305600 | Oryza sativa | 190 | 7.00E−47 |
| ABD32318.1 | mitochondrial import inner | Oryza sativa | 190 | 9.00E−47 |
| | membrane translocase | | | |
| | subunit Tim17/Tim22/ | | | |
| | Tim23 family protein | | | |
| EAY89687.1 | hypothetical protein | Oryza sativa | 188 | 3.00E−46 |
| | OsI_010920 | | | |
| NP_849394.1 | protein translocase/ | Arabidopsis thaliana | 178 | 3.00E−43 |
| | protein transporter | | | |
| AAM65873.1 | protein translocase/ | Arabidopsis thaliana | 178 | 3.00E−43 |
| | protein transporter | | | |
| NP_567488.1 | hypothetical protein | Oryza sativa | 178 | 3.00E−43 |
| | OsI_010920 | | | |
| EAZ26649.1 | hypothetical protein | Oryza sativa | 164 | 4.00E−39 |
| | OsI_010920 | | | |
| CAB10395.1 | pore protein homolog | Arabidopsis thaliana | 148 | 3.00E−34 |
| AAT45008.1 | stress-inducible membrane | Xerophyta humilis | 136 | 2.00E−30 |
| | pore-like protein | | | |
| CAK26794.1 | hypothetical protein | Sporobolus stapfianus | 134 | 5.00E−30 |
| NP_001054446.1 | Os05g0111200 | Oryza sativa | 91 | 7.00E−17 |
| CAA09867.1 | amino acid selective | Hordeum vulgare | 89 | 4.00E−16 |
| | channel protein | subsp. vulgure | | |
| EAY96269.1 | hypothetical protein | Oryza sativa | 87 | 8.00E−16 |
| | OsI_017502 | | | |
| CAA97910.1 | core protein | Pisum sativum | 84 | 7.00E−15 |
| CAA63967.1 | pom14 | Solanum tuberosum | 84 | 7.00E−15 |
| NP_180456.1 | protein translocase/ | Arabidopsis thaliana | 80 | 1.00E−13 |
| | protein transporter | | | |

### Example 17

### Identification and Isolation of the Promoter Region

For our promoter identification purposes, the sequence upstream of the start codon of Zm.8705.1.S1_at gene was defined as the promoter pZmNP28_{—}655. To isolate this predicted promoter region, the sequence of EST ZM06MC34918_{—}62096753 was mapped to the BPS in-house maize genomic DNA sequence database. One maize genomic DNA sequence of 1697 bp, ZmGSStuc11-12-04.119561.1 was identified to harbour the EST ZM06MC34918_{—}62096753 in an antisense direction. The 1697 by sequence of the ZmGSStuc11-12-04.119561.1 shown in SEQ ID NO: 196 contains a complete coding sequence (CDS) of the gene and a 655 bp upstream sequence of the start codon ATG including a 140 bp putative 5″UTR based on the sequence alignment result to the Zea mays mRNA clone EL01N0448C02.c sequence (GenBank accession: BTO17732.1). This 656 bp was designated as pZmNP28_{—}655 and cloned by PCR using the following specific primers:

Forward primer:

(SEQ ID NO: 188)

AAAAGTAGCAATTGGGATAAC
Reverse primer:

(SEQ ID NO: 189)

GCTCGTCAGCTCGTCCTTGAG

The CDS sequence shown in SEQ ID NO: 36 was identified by Vector NTI software package as a gene encoding a protein that is homologous to the stress-inducible membrane pore protein of Bromus inermis (GenBank accession: AAL23749.1, Table 59). The translated amino acid sequence of the CDS is shown in SEQ ID NO: 54 and sequence of pZmNP28_{—}655 is shown as part of SEQ ID NO: 18 (nucleotide 1459 to nucleotide 2112).

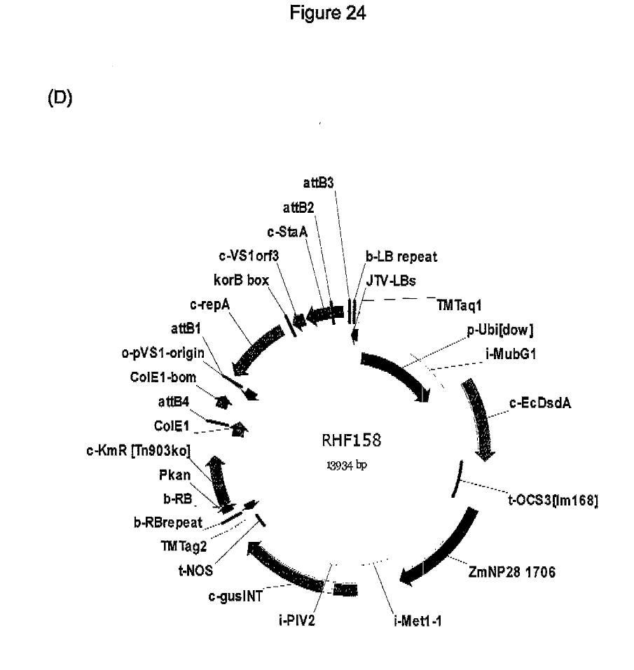

To obtain more sequence information of further upstream of pZmNP28_{—}655, GenomeWalk was conducted. Maize genomic DNA was extracted from Zea mays B73 and digested with the blunt end restriction enzymes Sspl, ScaI, EcoRV, StuI, DraI, to generate Genome Walker™ maize DNA libraries. Digested DNA was purified with phenol/chloroform and re-dissolved in TE buffer (10 mM Tris HCl, 0.1 mM EDTA, pH 8.0) prior to ligation to the Genome Walker™ adapters following the manufacturer's instruction (Clontech Laboratories, Inc, Mountain View, Calif., USA). Nested PCR was performed using Genome Walker™ library template with adapter and sequence specific primers. The GenomeWalk reactions produced a 2771 bp fragment containing 270 bp overlap with the 5′ end of pZmNP28_{—}655. The agarose gel showing this fragment was imaged as in FIG. 23. The fragment of Lane 6 of FIG. 23 was purified, cloned into a TOPO TA vector, pCR2.1-TOPO (Invitrogen, Carlsbad, Calif. 92008, USA) and sequenced. A contig sequence containing this 2771 bp fragment combined with pZmNP28_{—}655 was assembled and the resulted 3177 bp contig sequence is shown in SEQ ID NO: 90.

### Example 18

### Identification of the Longer Promoter Regions

To determine the promoter region, we isolated 3 more fragment with different lengths based on above contig sequence information for evaluation of their function as a promoter:

1). A 2070 bp fragment designated as pZmNP28_{—}2070 and isolated by PCR using the following specific primers:

Forward primer:

(SEQ ID NO: 190)

CTAGGTTGGTGAGATCCTTAG
Reverse primer:

(SEQ ID NO: 191)

CATCTTCTTCGACGCCTGTTC

Sequence of pZmNP28_{—}2070 is shown in SEQ ID NO:18
2). A 1706 bp fragment designated as pZmNP28_{—}1706 and isolated by PCR using the following specific primers:
Forward primer: GTGGCAGCTCTGAAGACTCCAAC (SEQ ID NO: 192)
Reverse primer: TGAGGCCGAGGCACTACGTCATG (SEQ ID NO: 193)
Compared to pZmNP28_{—}2070, pZmNP28_{—}1706 has a deletion of 326 bp at its 3′ end of the pZmNP28_{—}2070. Sequence of pZmNP28_{—}1706 is shown as part of SEQ ID NO: 18.
3). A 507 bp fragment designated as pZmNP28_{—}507 and isolated by PCR using the following specific primers:

Forward primer:

(SEQ ID NO: 194)

TGACGTTTGTGTAATTGGGCTTG
Reverse primer:

(SEQ ID NO: 195)

GCTCGTCAGCTCGTCCTTGAG

### Example 19

### BlastN Results of the Longest Promoter Region pZmNP28_{—}2070

The 2113 bp region from the 5′ end of pZmNP28_{—}2070 to immediate upstream of the ATG was searched via BlastN. A few homologues to the 3′ end of this region were found and listed in Table 60.

TABLE 60
BlastN results of the 2113 bp region including ZmNP28_2070

| | | | |
|---|---|---|---|
| NM_001159134.1 | Zea mays LOC100286246 | 202 | 7.00E−48 |
| | (gpm462), mRNA > | | |
| | gb\|EU976384.1\|Zea | | |
| EU968359.1 | Zea mays clone 319482 | 202 | 7.00E−48 |
| | stress-inducible membrane | | |
| | pore protein mRNA, complete | | |
| | cds | | |
| EU953175.1 | Zea mays clone 1389131 | 202 | 7.00E−48 |
| | mRNA sequence | | |
| AY111174.1 | Zea mays CL27726_1 mRNA | 196 | 3.00E−46 |
| | sequence | | |
| DQ245984.1 | Zea mays clone 93911 mRNA | 195 | 1.00E−45 |
| | sequence | | |

### Example 20

### PLACE Analysis of the Longest Promoter Region pZmNP28_{—}2070

Cis-acting motifs in the 2113 bp region from the 5′ end of pZmNP28_{—}2070 to immediate upstream of the ATG were identified using PLACE (a database of Plant Cis-acting Regulatory DNA elements) via Genomatix. The Results are listed in Table 61.

TABLE 61
PLACE analysis results of the 656 bp ZmNP19 promoter

| | | | | |
|---|---|---|---|---|
| IUPAC | Start pos. | End pos. | Strand | Sequence |

| | | | | |
|---|---|---|---|---|
| MYBPLANT | 1 | 11 | − | CACCAACCTAG |
| PALBOXLPC | 4 | 14 | − | TCTCACCAACC |
| BOXLCOREDCPAL | 4 | 10 | − | ACCAACC |
| TAAAGSTKST1 | 33 | 39 | − | CATAAAG |
| SORLIP1AT | 37 | 49 | − | AGAGCTGCCACAT |
| RAV1AAT | 58 | 62 | + | CAACA |
| CATATGGMSAUR | 86 | 91 | + | CATATG |
| CATATGGMSAUR | 86 | 91 | − | CATATG |
| GT1GMSCAM4 | 105 | 110 | + | GAAAAA |
| OSE1ROOTNODULE | 152 | 158 | − | AAAGATG |
| TAAAGSTKST1 | 159 | 165 | − | CCTAAAG |
| TBOXATGAPB | 194 | 199 | + | ACTTTG |
| CCAATBOX1 | 203 | 207 | + | CCAAT |
| OSE2ROOTNODULE | 227 | 231 | − | CTCTT |
| AMMORESIIUDCRNIA1 | 231 | 238 | + | GGTAGGGT |
| MYBPZM | 231 | 237 | − | CCCTACC |
| CCAATBOX1 | 247 | 251 | + | CCAAT |
| CIACADIANLELHC | 268 | 277 | + | CAATAAAATC |
| IBOXCORENT | 276 | 282 | − | GATAAGA |
| P1BS | 283 | 290 | + | GAATATCC |
| P1BS | 283 | 290 | − | GGATATTC |
| MYBST1 | 285 | 291 | − | GGGATAT |
| OSE2ROOTNODULE | 310 | 314 | − | CTCTT |
| -10PEHVPSBD | 322 | 327 | + | TATTCT |
| BOXIINTPATPB | 324 | 329 | − | ATAGAA |
| PYRIMIDINEBOXOSRAM | 333 | 338 | − | CCTTTT |
| -10PEHVPSBD | 361 | 366 | + | TATTCT |
| MYB1LEPR | 372 | 378 | + | GTTAGTT |
| -300CORE | 386 | 394 | + | TGTAAAGAC |
| TAAAGSTKST1 | 386 | 392 | + | TGTAAAG |
| IBOXCORE | 427 | 433 | + | GATAAAG |
| TAAAGSTKST1 | 427 | 433 | + | GATAAAG |
| BIHD1OS | 448 | 452 | − | TGTCA |
| RAV1AAT | 451 | 455 | + | CAACA |
| GT1GMSCAM4 | 456 | 461 | − | GAAAAA |
| TBOXATGAPB | 462 | 467 | − | ACTTTG |
| TAAAGSTKST1 | 468 | 474 | + | ATTAAAG |
| OSE2ROOTNODULE | 472 | 476 | − | CTCTT |
| CCAATBOX1 | 487 | 491 | + | CCAAT |
| DPBFCOREDCDC3 | 511 | 517 | + | ACACAAG |
| MYBST1 | 516 | 522 | + | AGGATAT |
| SEF3MOTIFGM | 562 | 567 | − | AACCCA |
| AACACOREOSGLUB1 | 565 | 571 | − | AACAAAC |
| SORLIP1AT | 570 | 582 | + | TTCCTCGCCACTC |
| DPBFCOREDCDC3 | 630 | 636 | + | ACACTAG |
| ELRECOREPCRP1 | 661 | 675 | − | TTTGACCTAAATAAG |
| QELEMENTZMZM13 | 666 | 680 | + | TTAGGTCAAACTATC |
| SORLIP2AT | 682 | 692 | − | GGGGCCATGAA |
| TAAAGSTKST1 | 692 | 698 | − | TATAAAG |
| WBBOXPCWRKY1 | 695 | 709 | − | TTTGACTATACTATA |
| PYRIMIDINEBOXHVEPB | 710 | 717 | − | TTTTTTCC |
| GT1GMSCAM4 | 711 | 716 | + | GAAAAA |
| ANAERO1CONSENSUS | 715 | 721 | + | AAACAAA |
| CAREOSREP1 | 749 | 754 | + | CAACTC |
| CPBCSPOR | 762 | 767 | + | TATTAG |
| GT1CORE | 777 | 787 | − | AGGTTAAGGAC |
| PYRIMIDINEBOXOSRAM | 785 | 790 | + | CCTTTT |
| ATHB1ATCONSENSUS | 809 | 817 | + | CAATAATTG |
| ATHB1ATCONSENSUS | 809 | 817 | − | CAATTATTG |
| S1FSORPL21 | 818 | 825 | − | ATGGTATT |
| S1FBOXSORPS1L21 | 820 | 825 | − | ATGGTA |
| CCAATBOX1 | 851 | 855 | + | CCAAT |
| ATHB6COREAT | 852 | 860 | + | CAATTATTA |
| PREATPRODH | 863 | 868 | + | ACTCAT |
| CCAATBOX1 | 871 | 875 | + | CCAAT |
| RAV1AAT | 883 | 887 | − | CAACA |
| WBOXHVISO1 | 885 | 899 | − | AGTGACTAATGACAA |
| BIHD1OS | 886 | 890 | + | TGTCA |
| DPBFCOREDCDC3 | 928 | 934 | − | ACACGAG |
| 2SSEEDPROTBANAPA | 929 | 937 | − | CAAACACGA |
| CCAATBOX1 | 944 | 948 | + | CCAAT |
| QELEMENTZMZM13 | 951 | 965 | + | CTAGGTCATGTTTGG |
| SITEIIATCYTC | 963 | 973 | + | TGGGCTCCACT |
| CIACADIANLELHC | 994 | 1003 | + | CAACATGATC |
| RAV1AAT | 994 | 998 | + | CAACA |
| RAV1AAT | 1016 | 1020 | − | CAACA |
| WBOXATNPR1 | 1017 | 1031 | + | GTTGACTAAAGACCT |
| TAAAGSTKST1 | 1021 | 1027 | + | ACTAAAG |
| WBOXHVISO1 | 1053 | 1067 | + | CATGACTTCGCTCAA |
| SURECOREATSULTR11 | 1079 | 1085 | − | GAGACTA |
| MYCATERD | 1127 | 1133 | − | CATGTGG |
| MYCATRD2 | 1128 | 1134 | + | CACATGT |
| -300ELEMENT | 1138 | 1146 | + | TGCAAAGGG |
| CGACGOSAMY3 | 1169 | 1173 | − | CGACG |
| TRANSINITDICOTS | 1173 | 1180 | − | AAGATGGC |
| OSE1ROOTNODULE | 1175 | 1181 | − | AAAGATG |
| TAAAGSTKST1 | 1185 | 1191 | − | GGTAAAG |
| IBOXCORE | 1218 | 1224 | − | GATAATA |
| SREATMSD | 1219 | 1225 | + | ATTATCC |
| MYBST1 | 1220 | 1226 | − | GGGATAA |
| CGCGBOXAT | 1226 | 1231 | + | CCGCGT |
| CGCGBOXAT | 1226 | 1231 | − | ACGCGG |
| MYBPZM | 1240 | 1246 | + | CCCTACC |
| HBOXCONSENSUSPVCHS | 1241 | 1261 | + | CCTACCCTAAACACTATGGGC |
| RAV1AAT | 1261 | 1265 | + | CAACA |
| CGACGOSAMY3 | 1267 | 1271 | − | CGACG |
| SURECOREATSULTR11 | 1303 | 1309 | − | GAGACCT |
| MYBCOREATCYCB1 | 1314 | 1318 | − | AACGG |
| RAV1AAT | 1326 | 1330 | − | CAACA |
| CGACGOSAMY3 | 1373 | 1377 | − | CGACG |
| LTRECOREATCOR15 | 1425 | 1431 | − | TCCGACC |
| SORLIP5AT | 1440 | 1446 | + | GAGTGAG |
| INTRONLOWER | 1447 | 1452 | + | TGCAGG |
| CCAATBOX1 | 1469 | 1473 | − | CCAAT |
| MYBST1 | 1472 | 1478 | + | GGGATAA |
| SREATMSD | 1473 | 1479 | − | GTTATCC |
| IBOXCORE | 1474 | 1480 | + | GATAACA |
| GT1GMSCAM4 | 1485 | 1490 | + | GAAAAA |
| GT1CORE | 1513 | 1523 | + | GGGTTAAATAA |
| TATABOXOSPAL | 1516 | 1522 | − | TATTTAA |
| EECCRCAH1 | 1560 | 1566 | − | GATTTCC |
| IBOXCORE | 1568 | 1574 | − | GATAAAT |
| OSE1ROOTNODULE | 1571 | 1577 | − | AAAGATA |
| CCAATBOX1 | 1590 | 1594 | − | CCAAT |
| RBCSCONSENSUS | 1591 | 1597 | − | AATCCAA |
| TGACGTVMAMY | 1602 | 1614 | + | GATTTTGACGTTT |
| HEXMOTIFTAH3H4 | 1604 | 1616 | − | ACAAACGTCAAAA |
| WBOXATNPR1 | 1605 | 1619 | + | TTTGACGTTTGTGTA |
| CCAATBOX1 | 1620 | 1624 | − | CCAAT |
| SITEIIATCYTC | 1622 | 1632 | + | TGGGCTTGACA |
| WBOXATNPR1 | 1626 | 1640 | + | CTTGACAGCCCCATC |
| BIHD1OS | 1628 | 1632 | − | TGTCA |
| LTRE1HVBLT49 | 1661 | 1666 | − | CCGAAA |
| SITEIIATCYTC | 1675 | 1685 | − | TGGGCCGAATC |
| MYBST1 | 1689 | 1695 | + | AGGATAG |
| RAV1AAT | 1707 | 1711 | + | CAACA |
| TGACGTVMAMY | 1720 | 1732 | + | GTCCATGACGTAG |
| HEXMOTIFTAH3H4 | 1722 | 1734 | − | CACTACGTCATGG |
| SITEIIATCYTC | 1742 | 1752 | − | TGGGCTTTGAG |
| TATABOX3 | 1757 | 1763 | − | TATTAAT |
| MYBST1 | 1761 | 1767 | − | TGGATAT |
| TATCCAYMOTIFOSRAMY | 1762 | 1768 | + | TATCCAC |
| TATCCACHVAL21 | 1762 | 1768 | + | TATCCAC |
| ACGTABOX | 1772 | 1777 | + | TACGTA |
| ACGTABOX | 1772 | 1777 | − | TACGTA |
| WBOXHVISO1 | 1785 | 1799 | − | AGTGACTCCCTCGGC |
| WBOXNTCHN48 | 1793 | 1807 | − | ACTGACTCAGTGACT |
| GCN4OSGLUB1 | 1798 | 1806 | + | CTGAGTCAG |
| MYBCOREATCYCB1 | 1812 | 1816 | + | AACGG |
| UPRMOTIFIIAT | 1832 | 1850 | + | CCGTGTGCCGGTGTCCACG |
| DPBFCOREDCDC3 | 1839 | 1845 | − | ACACCGG |
| CGCGBOXAT | 1848 | 1853 | + | ACGCGC |
| CGCGBOXAT | 1848 | 1853 | − | GCGCGT |
| CGCGBOXAT | 1850 | 1855 | + | GCGCGC |
| CGCGBOXAT | 1850 | 1855 | − | GCGCGC |
| UPRMOTIFIIAT | 1855 | 1873 | + | CCCCGGTGCGGCCGCCACG |
| SORLIP1AT | 1862 | 1874 | + | GCGGCCGCCACGA |
| GCCCORE | 1864 | 1870 | + | GGCCGCC |
| CGCGBOXAT | 1875 | 1880 | + | CCGCGG |
| CGCGBOXAT | 1875 | 1880 | − | CCGCGG |
| GADOWNAT | 1878 | 1890 | − | CGGCACGTGTCCG |
| CACGTGMOTIF | 1879 | 1891 | + | GGACACGTGCCGG |
| DPBFCOREDCDC3 | 1881 | 1887 | + | ACACGTG |
| SORLIP2AT | 1889 | 1899 | + | CGGGCCTCGCA |
| DPBFCOREDCDC3 | 1899 | 1905 | + | ACACGCG |
| CGCGBOXAT | 1901 | 1906 | + | ACGCGT |
| CGCGBOXAT | 1901 | 1906 | − | ACGCGT |
| SORLIP2AT | 1909 | 1919 | − | GGGGCCGTGGG |
| UPRMOTIFIIAT | 1935 | 1953 | + | CCGCGGTGCCCGCGCCACG |
| CGCGBOXAT | 1935 | 1940 | + | CCGCGG |
| CGCGBOXAT | 1935 | 1940 | − | CCGCGG |
| SORLIP1AT | 1942 | 1954 | + | GCCCGCGCCACGG |
| CGCGBOXAT | 1944 | 1949 | + | CCGCGC |
| CGCGBOXAT | 1944 | 1949 | − | GCGCGG |
| REBETALGLHCB21 | 1980 | 1986 | + | CGGATAG |
| CGACGOSAMY3 | 1999 | 2003 | + | CGACG |
| HEXAMERATH4 | 1999 | 2004 | − | CCGTCG |
| DRE2COREZMRAB17 | 2003 | 2009 | − | ACCGACC |
| MYBCOREATCYCB1 | 2013 | 2017 | − | AACGG |
| SORLIP1AT | 2021 | 2033 | − | TGTCTCGCCACTC |
| ARFAT | 2028 | 2034 | − | CTGTCTC |
| SURECOREATSULTR11 | 2028 | 2034 | + | GAGACAG |
| SEBFCONSSTPR10A | 2028 | 2034 | − | CTGTCTC |
| BS1EGCCR | 2033 | 2038 | + | AGCGGG |
| CGACGOSAMY3 | 2058 | 2062 | + | CGACG |
| TCA1MOTIF | 2063 | 2072 | − | TCATCTTCTT |
| DPBFCOREDCDC3 | 2082 | 2088 | + | ACACGCG |
| CGCGBOXAT | 2084 | 2089 | + | ACGCGG |
| CGCGBOXAT | 2084 | 2089 | − | CCGCGT |
| ASF1MOTIFCAMV | 2101 | 2113 | + | CGAGCTGACGAGC |

### Example 21

### Binary Vector Construction for Maize Transformation

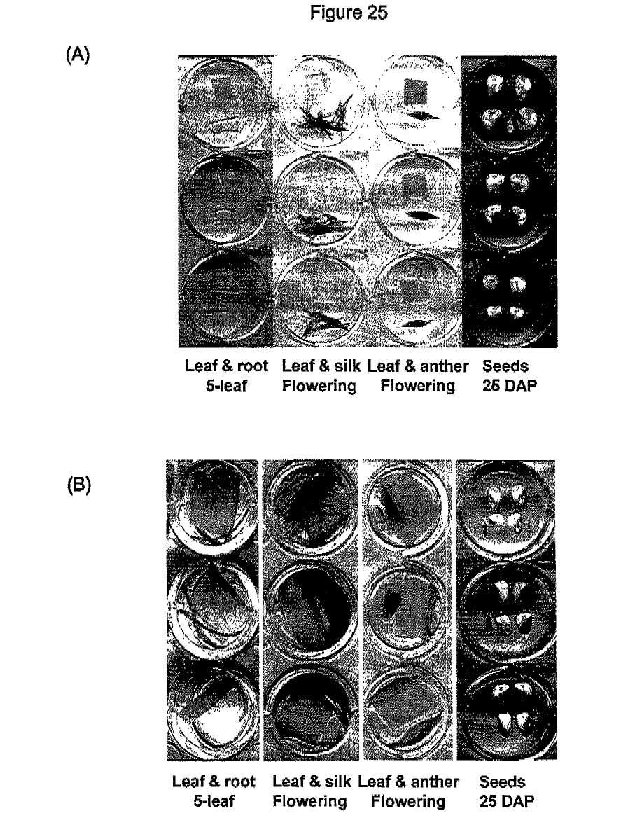

For pZmNP28_{—}655 and pZmNP28_{—}507, the promoter fragments obtained from PCR were cloned into pENTR™ 5′-TOPO TA Cloning vector (Invitrogen, Carlsbad, Calif., USA). An intron-mediated enhancement (IME)-intron (BPSI.1) was inserted into the restriction enzyme BsrGI site that is 24 bp downstream of the 3′ end of the pZmNP28_{—}655 and pZmNP28_{—}507. The resulting vector was used as a Gateway entry vector in order to produce the final binary vector RLN 90 and RLN 93 for maize transformation, which comprises a plant selectable marker expression cassette (p-Ubi::AHAS::t-NOS) as well as a promoter evaluation cassette that consists testing promoter, MET1-1 intron to supply intron-mediated enhancement in monocot cells, GUS reporter gene, and NOS terminator (FIGS. 24 A and B). For pZmNP28_{—}2070 and pZmNP28_{—}1706, the 2070 bp and the 1706 bp fragments were modified by the addition of a PacI restriction enzyme site at its 5′ end and a BsiWI site at its 3′ end. The PacI-pZmNP28_{—}2070-BsiWI and PacI-pZmNP28_{—}1706-BsiWI fragments were digested and ligated into a PacI and BsiWI digested BPS basic binary vector HF84. HF84 comprises a plant selectable marker expression cassette (p-Ubi::c-EcEsdA::t-NOS) as well as a promoter evaluation cassette that consists of a multiple cloning site for insertion of putative promoters via PacI and BsiWI, rice MET1-1 intron to supply intron-mediated enhancement in monocot cells, GUS reporter gene, and NOS terminator. The resulting binary vectors comprising the pZmNP28_{—}2070::i-MET1::GUS::t-NOS or pZmNP28_{—}1706::i-MET1::GUS::t-NOS expression cassette was named as RHF160 or RHF158 and were used maize transformation to evaluate the expression pattern driven by pZmNP28_{—}2070 or pZmNP1706. FIG. 24 C is a diagram of RHF160 and FIG. 24 D is a diagram of RHF158.

### Example 22

### Promoter Evaluation in Transgenic Maize with the Binary Vectors RLN90, RLN93, RHF158 and RHF160

Expression patterns and levels driven by the promoter were measured using GUS histochemical analysis following the protocol in the art (Jefferson 1987). Maize transformation was conducted using an Agrobacterium-mediated transformation system. Ten and five single copy events for T0 and T1 plants were used for the promoter analysis. GUS expression was measured at various developmental stages:

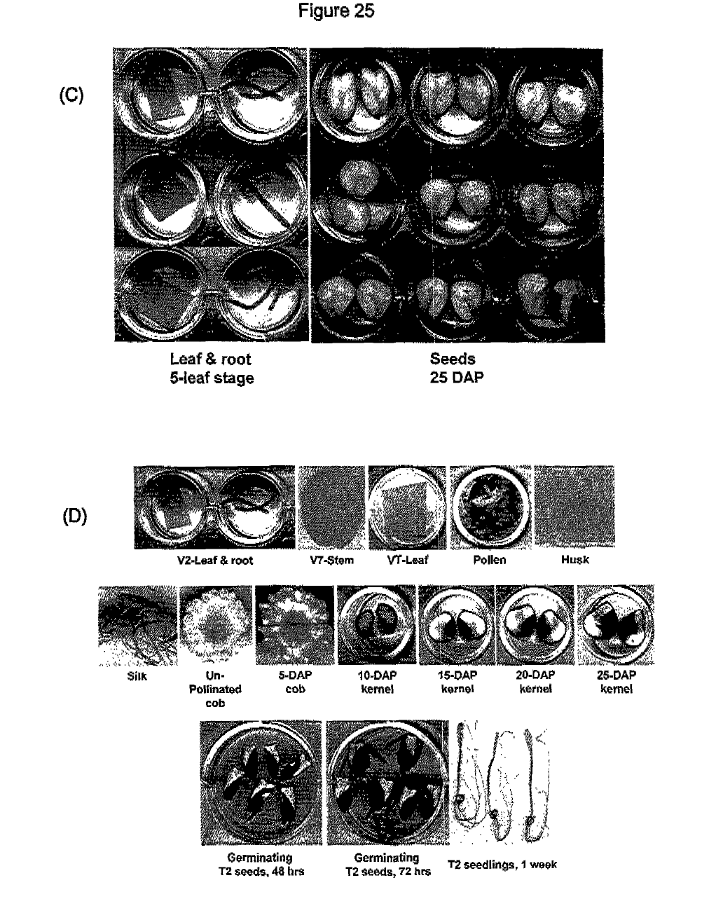

1) Roots and leaves at 5-leaf stage
2) Stem at V-7 stage
2) Leaves, husk and silk at flowering stage (first emergence of silk)
3) Spikelets/Tassel (at pollination)
5) Ear or Kernels at 5, 10, 15, 20, and 25 days after pollination (DAP)
The results indicated that pZmNP28_{—}655, pZmNP28_{—}507 and pZmNP28_{—}2070 expressed specifically in pollen and in embryo, and pZmNP28_{—}1706 did not express in any tested tissues (FIG. 25 A to D)).

### Example 23

### Core Sequences Driving the Embryo-Specific Expression of Promoter pZmNP28

The experiment results of expression evaluation driven by several fragments of this promoter region in different length as described as above showed that a 326 bp core sequence is critical to the embryo specific expression of this promoter. The promoter fragments, pZmNP28_{—}655, pZmNP28-507 and pZmNP28_{—}2070, which contain this core sequence, showed embryo specific expression (FIGS. 25 A, C and D). The promoter fragment pZmNP28_{—}1706, which does not contain this core sequence, showed no expression at all. The core sequence is shown in SEQ ID NO: 18 (in particular nucleotides 1745 to 2070 of SEQ ID NO:18)

1. An expression cassette for regulating seed-specific expression of a polynucleotide of interest, said expression cassette comprising a transcription regulating nucleotide sequence selected from the group consisting of:
(a) the nucleic acid sequence of SEQ ID NO: 1, 2, 3, 4, 5, 6, 7, 8, 9, 10, 11, 12, 13, 14, 15, 16, 17, or 18, or a variant thereof;
(b) a nucleic acid sequence which is at least 80% identical to the nucleic acid sequence of SEQ ID NO: 1, 2, 3, 4, 5, 6, 7, 8, 9, 10, 11, 12, 13, 14, 15, 16, 17, or 18;
(c) a nucleic acid sequence which hybridizes under stringent conditions to the nucleic acid sequence of SEQ ID NO: 1, 2, 3, 4, 5, 6, 7, 8, 9, 10, 11, 12, 13, 14, 15, 16, 17, or 18;
(d) a nucleic acid sequence which hybridizes to a nucleic acid sequence located upstream of the open reading frame sequence of SEQ ID NO: 19, 20, 21, 22, 23, 24, 25, 26, 27, 28, 29, 30, 31, 32, 33, 34, 35 or 36;
(e) a nucleic acid sequence which hybridizes to a nucleic acid sequence located upstream of an open reading frame sequence encoding the amino acid sequence of SEQ ID NO: 37, 38, 39, 40, 41, 42, 43, 44, 45, 46, 47, 48, 49, 50, 51, 52, 53 or 54;
(f) a nucleic acid sequence which hybridizes to a nucleic acid sequence located upstream of an open reading frame sequence that is at least 80% identical to the open reading frame sequence of SEQ ID NO: 19, 20, 21, 22, 23, 24, 25, 26, 27, 28, 29, 30, 31, 32, 33, 34, 35 or 36, wherein the open reading frame sequence encodes a seed protein;
(g) a nucleic acid sequence which hybridizes to a nucleic acid sequence located upstream of an open reading frame encoding an amino acid sequence that is at least 80% identical to the amino acid sequence of SEQ ID NO: 37, 38, 39, 40, 41, 42, 43, 44, 45, 46, 47, 48, 49, 50, 51, 52, 53 or 54, wherein the open reading frame sequence encodes a seed protein;
(h) a nucleic acid sequence obtained by 5′ genome walking or by thermal asymmetric interlaced polymerase chain reaction (TAIL-PCR) on genomic DNA from the first exon of the open reading frame sequence of SEQ ID NO: 19, 20, 21, 22, 23, 24, 25, 26, 27, 28, 29, 30, 31, 32, 33, 34, 35 or 36;
(i) a nucleic acid sequence obtained by 5′ genome walking or TAIL PCR on genomic DNA from the first exon of an open reading frame sequence that is at least 80% identical to the open reading frame sequence of SEQ ID NO: 19, 20, 21, 22, 23, 24, 25, 26, 27, 28, 29, 30, 31, 32, 33, 34, 35 or 36, wherein the open reading frame sequence encodes a seed protein; and
(j) a nucleic acid sequence obtained by 5′ genome walking or TAIL PCR on genomic DNA from the first exon of an open reading frame sequence encoding an amino acid sequence that is at least 80% identical to an amino acid sequence encoded by the open reading frame sequence of SEQ ID NO: 37, 38, 39, 40, 41, 42, 43, 44, 45, 46, 47, 48, 49, 50, 51, 52, 53 or 54, wherein the open reading frame sequence encodes a seed protein.

2. The expression cassette of claim 1, wherein the expression cassette further comprises at least one polynucleotide of interest being operatively linked to the transcription regulating nucleotide sequence.

3. The expression cassette of claim 2, wherein the at least one polynucleotide of interest is heterologous to the transcription regulating nucleotide sequence.

4. A vector comprising the expression cassette of claim 1.

5. The vector of claim 4, wherein the vector is an expression vector.

6. A host cell comprising the expression cassette of claim 1 or a vector comprising said expression cassette.

7. The host cell of claim 6, wherein the host cell is a plant cell.

8. A transgenic plant tissue, plant organ, plant, or seed comprising the expression cassette of claim 1 or a vector comprising said expression cassette.

9. The transgenic plant tissue, plant organ, plant, or seed of claim 8, wherein the plant is a monocot, or the transgenic plant tissue, plant organ, or seed is from a monocot.

10. A method for producing a transgenic plant tissue, plant organ, plant, or seed comprising:
(a) introducing the expression cassette of claim 1 or a vector comprising said expression cassette into a plant cell; and
(b) regenerating said plant cell to form a plant tissue, plant organ, plant, or seed.

11. A method for producing a transgenic plant tissue, plant organ, plant, or seed comprising:
(a) integrating the expression cassette of claim 1 or a vector comprising said expression cassette into the genome of a plant cell;
(b) regenerating said plant cell to form a plant tissue, plant organ, plant, or seed; and
(c) selecting said plant tissue, plant organ, plant, or seed for the presence of said expression cassette or a vector comprising said expression cassette.
